(19) United States
(12) Patent Application Publication
(10) Pub. No.: US 2026/0334439 A1
(43) Pub. Date: Oct. 29, 2026

(54) BCAT2 INHIBITORS

(71) Applicant: Agios Pharmaceuticals, Inc., Cambridge, MA (US)

(72) Inventors: Barden Chan, Brookline, MA (US); Heather J. Finlay, Skillman, NJ (US); Peng Liu, Belmont, MA (US); Michael McVay, Melrose, MA (US); Mina C. Nakhia, Ottawa (CA)

(21) Appl. No.: 19/483,686

(22) PCT Filed: May 16, 2024
(86) PCT No.: PCT/US2024/029738

§ 371 Date: Nov. 12, 2025

Related U.S. Application Data
(60) Provisional application No. 63/467,125, filed on May 17, 2023.

(51) Int. Cl. A01K 67/0275; A61K 31/4196; A61K 31/444; A61P 3/00; C07D 401/14; C07D 403/14; C07D 471/04; C12N 9/22; C12N 15/113

(52) U.S. Cl. CPC A01K 67/0275; A61K 31/4196; A61K 31/444; A61P 3/00; C07D 401/14; C07D 403/14; C07D 471/04; C12N 9/226; C12N 15/113; A01K 2217/075; A01K 2217/15; A01K 2227/105; A01K 2267/0306; C12N 2310/20

(57) ABSTRACT

The disclosure is directed to compounds, and pharmaceutically acceptable salts thereof, that inhibit branched-chain amino acid aminotransferase 2 (BCAT2), pharmaceutical compositions comprising those compounds, and methods of using those compounds for treating branched chain amino acid (BCAA)-catabolism-related diseases. The disclosure is also directed to in vivo models for the study of disorders characterized by defects in the metabolism of BCAAs.

### RELATED APPLICATIONS

This application claims the benefit of priority to U.S. Provisional Application No. 63/467,125, filed May 17, 2023, the entire contents of which are incorporated herein by reference.

### REFERENCE TO ELECTRONIC SEQUENCE LISTING

The application contains a Sequence Listing which has been submitted electronically in .XML format and is hereby incorporated by reference in its entirety. Said .XML copy, created on May 15, 2024, is named “128748-02920.xml” and is 56,094 bytes in size. The sequence listing contained in this .XML file is part of the specification and is hereby incorporated by reference herein in its entirety.

### TECHNICAL FIELD

This application pertains to compounds that inhibit branched-chain amino acid aminotransferase 2 (BCAT2), pharmaceutical compositions comprising those compounds, and methods of using those compounds for treating branched chain amino acid (BCAA)-catabolism-related diseases.

### BACKGROUND

Leucine, isoleucine, and valine are essential branched chain amino acids (BCAAs). BCAAs are catabolized by enzymatic conversions that result in transformation of these amino acids into various metabolites. FIG. 1 shows the catabolism pathways for leucine, isoleucine, and valine.

When genetic mutations result in defects in the enzymes that facilitate the BCAA catabolic conversions (e.g., loss of function mutations), toxic levels of catabolic intermediates accumulate, resulting in organic acidemias. Propionic acidemia, for example, results from loss of function mutations in propionyl-CoA carboxylase. Methylmalonic acidemia results from deficiency of methylmalonyl CoA mutase, defective enzymes (MMADHC or MMAA/B) used in the synthesis of cofactor 5′-adenosylcobalamin, or deficient activity of methylmalonyl-CoA epimerase. Maple syrup urine disease (MSUD), isovaleric acidemia (IVA), propionic acidemia (PA), and methylmalonic acidemia (MMA) are some of the most common organic acidemias.

The accumulation of toxic levels of catabolic intermediates lead to a variety of pathologic disease phenotypes. For example, acute metabolic decompensations (AMDs) are induced as a result of a catabolic stressor which triggers protein catabolism and increases the production of these toxic metabolites, resulting in additional clinical distress. AMDs contribute to the neurological deficits in patients and put them at risk of stroke, coma, and death. Patients with MMA and PA, for example, experience frequent episodes of metabolic decompensations, especially in the first years of life. Those patients can suffer from a number of complications and impairments: potentially life-threatening episodes of acidosis, hyperammonemia, pancreatitis, metabolic stroke, cardiomyopathy, bone marrow suppression, seizures, and encephalopathy (which can accompany acutely deranged metabolism). The long-term cognitive outcome of patients with PA and MMA is negatively correlated to the number of metabolic decompensations they experience. Patients with PA also often experience significant cardiac issues. Intellectual impairment can be a major secondary presentation of both PA and MMA. Convulsions are a hallmark neurological alteration seen in children with MMA. Patients with MMA also often experience renal insufficiency.

There is currently no approved therapy on the market for either PA or MMA, except for CARBAGLU®, which is approved to treat hyperammonemia seen in MMA and PA. Liver transplantation may protect against metabolic instability but is not curative and patients remain at risk for long-term complications. Hepatic-specific gene therapy is the main treatment modality under development, but is not expected to be curative because it relies on the same mechanism of action as liver transplantation. Thus, a need exists for effective therapies for treating PA, MMA, and other BCAA-catabolism-related diseases.

The first step in BCAA catabolism is common to the catabolism of each of leucine, isoleucine, and valine (the BCAAs), i.e., an enzymatic conversion mediated by BCAA aminotransferase 2 (BCAT2). Subsequent catabolism of the metabolites follows distinct pathways. Inhibition of BCAT2 will lower BCAA pathway flux and should significantly reduce the level of toxic metabolites seen in patients with BCAA-catabolism-related diseases. Thus, a need exists for effective BCAT2 inhibitors for treating BCAA-catabolism-related diseases.

### SUMMARY

The disclosure provides compounds that are BCAT2 inhibitors, pharmaceutical compositions comprising those BCAT2 inhibitors, and methods of treating BCAA-catabolism-related diseases using those BCAT2 inhibitors.

In some aspects, the disclosure is directed to compounds of Formula I or Formula II:

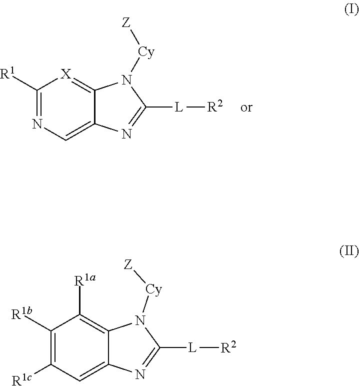

or a pharmaceutically acceptable salt thereof, wherein:
R¹ is optionally substituted heteroaryl, optionally substituted aryl, or optionally substituted 4-, 5- or 6-membered heterocycloalkyl;
R^{1a} is hydrogen or fluoro;
R^{1b} is optionally substituted heteroaryl, optionally substituted aryl, or optionally substituted 4-, 5- or 6-membered heterocycloalkyl;
R^{1c} is hydrogen, halo, C₁-C₆alkyl, C₁-C₆haloalkyl, C₁-C₆alkoxy, or C₁-C₆haloalkoxy;
X is CH or N;
Cy is optionally substituted C₅-C₇ cycloalkyl or optionally substituted 6- or 7-membered heterocycloalkyl;
Z is —NHC(O)Ar¹,

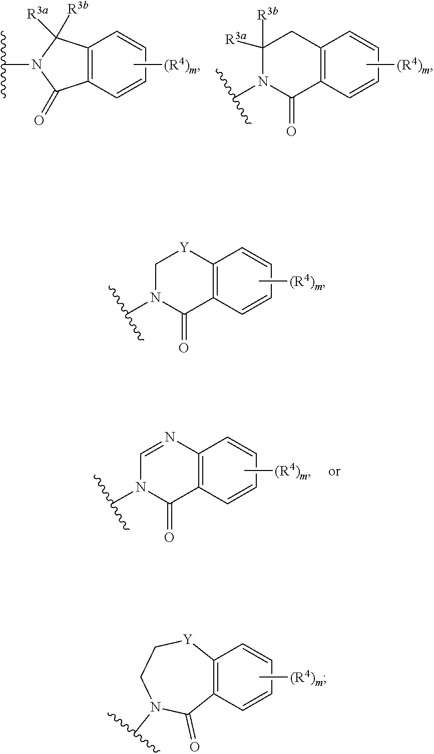

L is a bond, —O—, —NR^{a}—, or optionally substituted C₁-C₄alkylene;
   R^{a} is hydrogen or C₁-C₄alkyl;
R² is optionally substituted phenyl, optionally substituted pyridinyl, optionally substituted 5-membered heteroaryl, optionally substituted 4-, 5-, or 6-membered heterocycloalkyl, optionally substituted C₁-C₆alkyl, optionally substituted C₁-C₆alkoxy, optionally substituted C₁-C₆haloalkyl, optionally substituted C₁-C₆haloalkoxy, optionally substituted C₃-C₆cycloalkyl, or NR^{b}R^{c};
   R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl; and
Ar¹ is optionally substituted 6-membered heteroaryl, optionally substituted phenyl, or

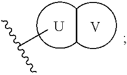

U is an optionally substituted phenyl or optionally substituted 6-membered heteroaryl;
V is an optionally substituted phenyl, optionally substituted 5- or 6-membered heterocycloalkyl, or optionally substituted 5- or 6-membered heteroaryl; and
wherein the point of attachment to the carbonyl group of —NHC(O) for variable Z occurs at the optionally substituted phenyl or optionally substituted 6-membered heteroaryl of U;
Y is O or NH;
R^{3a} is hydrogen, C₁-C₃alkyl, or oxo;
R^{3b} is hydrogen or C₁-C₃alkyl; or alternatively, R^{3a} and R^{3b} are taken together to form cyclopropyl;
each R⁴ is independently halo, cyano, C₁-C₆alkyl, C₁-C₆haloalkyl, or C₃-C₆cycloalkyl; and
m is 0, 1, 2, 3, or 4.

Stereoisomers and mixtures of stereoisomers of the compounds of Formula I and Formula II, and the pharmaceutical salts thereof, are also described. Methods of using compounds of Formula I and Formula II are described, as well as pharmaceutical compositions including the compounds of Formula I and Formula II.

### BRIEF DESCRIPTION OF THE DRAWINGS

FIG. 1 illustrates the BCAA catabolism pathways, the point of BCAT2 involvement, and the points of catabolic impairment resulting in methylmalonic acidemia (MMA) and propionyl acidemia (PA).

FIG. 2A shows plasma methylmalonic acid (MMA) levels in mouse models of methylmalonic acidemia comprising mutations in the gene encoding MMA-CoA mutase. The mice are either homozygous for an M698K mutation in MMA-CoA mutase (M698K/M698K), comprise a gene encoding M698K MMA-CoA mutase at a first locus and a knockout of the MMA-CoA mutase gene at a second locus (M698K/KO), or comprise a gene encoding M698K MMA-CoA mutase at a first locus and a gene encoding wildtype MMA-CoA mutase at a second locus (M698K/WT).

FIG. 2B shows urine methylmalonic acid (MMA) levels in mouse models of methylmalonic acidemia comprising mutations in the gene encoding MMA-CoA mutase. The mice are either homozygous for an M698K mutation in MMA-CoA mutase (M698K/M698K), comprise a gene encoding M698K MMA-CoA mutase at a first locus and a knockout of the MMA-CoA mutase gene at a second locus (M698K/KO), or comprise a gene encoding M698K MMA-CoA mutase at a first locus and a gene encoding wildtype MMA-CoA mutase at a second locus (M698K/WT).

FIG. 3 shows the probability of survival in BCAT2 knockout (KO) mice fed a regular diet or a low branched-chain amino acid (BCAA) diet.

FIG. 4 shows plasma propionyl carnitine levels in a mouse model of propionic acidemia that is homozygous for an A134T mutation in propionyl-CoA carboxylase subunit α (PCCA).

FIG. 5 shows plasma isoleucine levels for mice that are either homozygous for BCAT2 knockout (MUT^{WT/WT} BCAT2^{KO/KO}), comprise a gene encoding M698K MMA-CoA mutase at a first locus and a knockout of the MMA-CoA mutase gene at a second locus (MUT^{M698K/KO}BCAT2^{WT/WT}), or both (MUT^{M698K/KO}BCAT2^{KO/KO})

FIG. 6 shows plasma methylmalonic acid (MMA) levels for mice that are either homozygous for BCAT2 knockout (MUT^{WT/WT} BCAT2^{KO/KO}), comprise a gene encoding M698K MMA-CoA mutase at a first locus and a knockout of the MMA-CoA mutase gene at a second locus (MUT^{M698K/KO}BCAT2^{WT/WT}), or both (MUT^{M698K/KO}BCAT2^{KO/KO}).

FIG. 7 shows plasma 3-OH-isobutyric acid (3-HIB) levels for mice that are either homozygous for BCAT2 knockout (MUT^{WT/WT} BCAT2^{KO/KO}), comprise a gene encoding M698K MMA-CoA mutase at a first locus and a knockout of the MMA-CoA mutase gene at a second locus (MUT^{M698K/KO}BCAT2^{WT/WT}), or both (MUT^{M698K/KO}BCAT2^{KO/KO})

FIG. 8 shows plasma keto isoleucine (Keto-Ile) levels for mice that are either homozygous for BCAT2 knockout (MUT^{WT/WT} BCAT2^{KO/KO}), comprise a gene encoding M698K MMA-CoA mutase at a first locus and a knockout of the MMA-CoA mutase gene at a second locus (MUT^{M698K/KO}BCAT2^{WT/WT}), or both (MUT^{M698K/KO}BCAT2^{KO/KO})

FIG. 9 shows BCAT2 protein expression levels in muscle of mice homozygous (Homo) or heterozygous (Het) for a knockout of the mouse BCAT2 gene. BCAT2 protein expression levels were determined by Western blot and normalized to GAPDH protein levels.

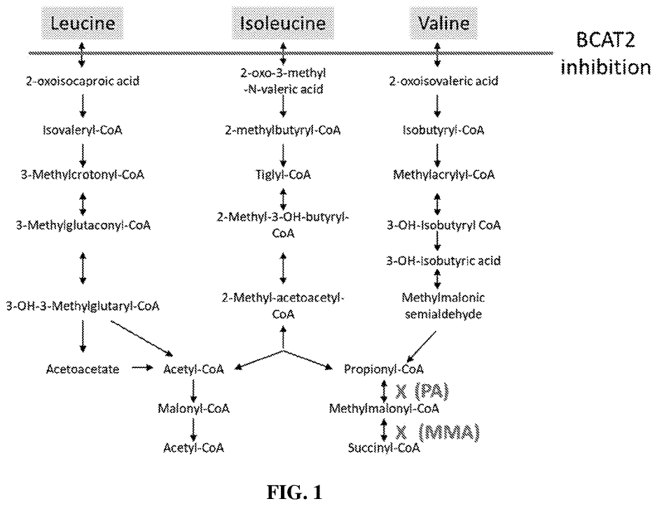

### DETAILED DESCRIPTION OF ILLUSTRATIVE EMBODIMENTS

### Definitions

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

As used in structures herein,

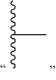

indicates the point of attachment of the particular depicted structure or substituent group to the appropriate atom(s) in the remainder of the molecule.

The articles “a” and “an,” as used herein and in the appended claims, are used herein to refer to one or to more than one (e.g., to at least one) of the grammatical object of the article unless the context clearly indicates otherwise. By way of example, “an element” means one element or more than one element.

“Pharmaceutically acceptable” means approved or approvable by a regulatory agency of the Federal or a state government or the corresponding agency in a country other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic and may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate and the like.

A “pharmaceutically acceptable excipient” refers to a substance that is non-toxic, biologically tolerable, and otherwise biologically suitable for administration to a subject, such as an inert substance added to a pharmacological composition or otherwise used as a vehicle, carrier, or diluent to facilitate administration of a compound of the disclosure and that is compatible therewith.

The term “alkyl,” when used alone or as part of a substituent group, refers to a straight- or branched-chain hydrocarbon group having from 1 to 12 carbon atoms (“C₁-C₁₂”), for example 1 to 6 carbons atoms (“C₁-C₆”), in the group. Examples of alkyl groups include methyl (C₁), ethyl (C₂), propyl (C₃) (e.g., n-propyl, isopropyl), butyl (C₄) (e.g., n-butyl, tert-butyl, sec-butyl, iso-butyl), pentyl (C₅) (e.g., n-pentyl, 3-pentanyl, amyl, neopentyl, 3-methyl-2-butanyl, tertiary amyl), hexyl (C₆) (e.g., n-hexyl), heptyl (C₇) (e.g., n-heptyl), octyl (C₈) (e.g., n-octyl), and the like. In some embodiments, the alkyl group is a C₁-C₆alkyl; in some embodiments, it is a C₁-C₄alkyl; and in some embodiments, it is a C₁-C₃alkyl.

The term “alkylene,” when used alone or as part of a substituent group, refers to an alkyl diradical, i.e., a straight- or branched-chain hydrocarbon group that is attached to two other groups. For example, one embodiment of a C₂alkylene is the diradical —CH₂CH₂—. In some embodiments, the alkylene group is C₁-C₄alkylene.

When a range of carbon atoms is used herein, for example, C₁-C₆, all ranges, as well as individual numbers of carbon atoms are encompassed. For example, “C₁-C₃” includes C₁-C₃, C₁-C₂, C₂-C₃, C₁, C₂, and C₃.

The term “cycloalkyl” when used alone or as part of a substituent group refers to cyclic-containing, non-aromatic hydrocarbon groups having from 3 to 10 carbon atoms (“C₃-C₁₀”), for example from 3 to 7 carbon atoms (“C₃-C₇”). Examples of cycloalkyl groups include cyclopropyl (C₃), cyclobutyl (C₄), cyclopentyl (C₅), cyclohexyl (C₆), cycloheptyl (C₇), and the like. In some embodiments, the cycloalkyl group is a C₃-C₄cycloalkyl; in some embodiments, it is a C₃-C₆cycloalkyl; and in some embodiments, it is C₅-C₇cycloalkyl.

The term “heterocycloalkyl” when used alone or as part of a substituent group refers to any three to twelve-membered monocyclic, saturated or partially unsaturated ring containing at least one heteroatom that is oxygen, nitrogen, or sulfur. The heterocycloalkyl group may be attached to another group or substituent through any heteroatom or carbon atom of the ring that results in a stable structure. In some embodiments, heterocycloalkyl rings are characterized by the number of ring atoms in the heterocycloalkyl group. For example, a 6-membered heterocycloalkyl group refers to a heterocycloalkyl group having 6 ring atoms in the group. Similarly, a 5-membered heterocycloalkyl group refers to a heterocycloalkyl group having 5 ring atoms in the group. Similarly, a 4-membered heterocycloalkyl group refers to a heterocycloalkyl group having 4 ring atoms in the group. The heterocycloalkyl moiety can be unsubstituted, or one or more of the carbon, nitrogen, or sulfur atoms in the ring can be substituted. Examples of heterocycloalkyl groups include azepanyl, aziridinyl, azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-lthiacyclohexyl, dithianyl, thiomorpholinyl, oxazepanyl, oxiranyl, oxetanyl, tetrahydrofuranyl, tetrahydropyranyl, tetrahydrothiophenyl, pyranyl, and the like. In some embodiments, the heterocycloalkyl is a three to twelve-membered monocyclic, saturated ring containing at least one heteroatom that is oxygen, nitrogen, or sulfur.

The terms “halo” or “halogen”, by itself or as part of a substituent group, means a fluorine, chlorine, bromine, or iodine atom.

The term “aryl” when used alone or as part of a substituent group also refers to a mono- or bicyclic-aromatic hydrocarbon ring structure having 6 or 10 carbon atoms in the ring, wherein one or more of the carbon atoms in the ring is optionally substituted. The term “aryl” also includes a mono- or bicyclic-aromatic hydrocarbon ring structure having 6 or 10 carbon atoms in the ring, wherein two adjacent carbon atoms in the ring are optionally substituted such that said two adjacent carbon atoms and their respective substituents form a cycloalkyl or heterocycloalkyl ring. Examples of aryl groups include phenyl, indenyl, naphthyl, 1, 2, 3,4-tetrahydronaphthyl, and the like.

The term “heteroaryl” when used alone or as part of a substituent group refers to a mono- or bicyclic-aromatic ring structure including carbon atoms as well as up to four heteroatoms that are each independently nitrogen, oxygen, or sulfur. Heteroaryl rings can include a total of 5, 6, 9, or 10 ring atoms. In some embodiments, heteroaryl rings are characterized by the number of ring atoms in the heteroaryl group. For example, a 6-membered heteroaryl group refers to a heteroaryl group having 6 ring atoms in the group. Similarly, a 5-membered heteroaryl group refers to a heteroaryl group having 5 ring atoms in the group. The heteroaryl moiety can be unsubstituted, or one or more of the carbon atoms or nitrogen atoms in the ring can be substituted. Examples of heteroaryls include pyridyl, pyrrolyl, pyrazinyl, pyrimidinyl, pyridazinyl, pyrazolyl, thienyl, indolyl, isoindolyl, imidazolyl, oxazolyl, isoxazolyl, thiazolyl, furyl, oxadiazolyl, thiadiazolyl, quinolyl, isoquinolyl, benzothiazolyl, benzoisothiazolyl, benzoxazolyl, benzoisoxazolyl, benzofuranyl, isobenzofuranyl, benzothiophenyl, benzoimidazolyl, indazolyl, quinoxalyl, quinazolinyl, triazolyl, tetrazolyl, isothiazolyl, pyranyl, purinyl, naphthyridinyl, phthalazinyl, cinnolinyl, pteridinyl, 1H-pyrrolo[2,3-b]pyridinyl, oxazolo[4,5-b]pyridinyl, oxazolo[5,4-b]pyridinyl, thiazolo[5,4-b]pyridinyl, thiazolo[4,5-b]pyridinyl, 3H-imidazo[4,5-b]pyridinyl, furo[2,3-b]pyridinyl, thieno[2,3-b]pyridinyl, pyrazolo[1,5-a]pyridinyl, imidazol[1,5-a]pyridinyl, and pyrrolo[1,2]pyridazinyl and the like. The term “heteroaryl” also includes pyridine, pyrimidine, and pyrazines groups having a fused optionally substituted cycloalkyl or fused optionally substituted heterocycloalkyl group. A pyridine, pyrimidine, or pyrazine having a fused optionally substituted cycloalkyl or fused optionally substituted heterocycloalkyl group may be attached to the remainder of the molecule at any available carbon atom on the pyridine, pyrimidine, or pyrazine ring. Exemplary heteroaryls having a fused optionally substituted cycloalkyl or fused optionally substituted heterocycloalkyl group include but are not limited to:

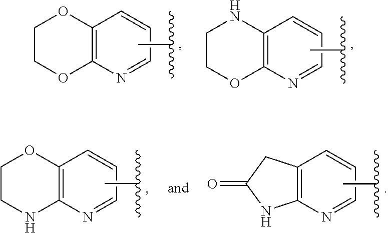

The term “optionally substituted,” as used herein to describe a chemical moiety defined herein, means that the moiety may, but is not required to be, substituted with one or more suitable functional groups or other substituents as provided herein. For example, a substituent may be optionally substituted with one or more of: halo (i.e., —F, —Cl, —Br, or —I), cyano, —NO₂, —N₃, —OH, —SH, C₁-C₆alkyl, C₃-C₆cycloalkyl, C₂-C₆alkenyl, C₂-C₆ alkynyl, C₁-C₆haloalkyl, C₁-C₆alkoxy, C₁-C₆ haloalkoxy, C₁-C₆alkylenethio, C₁-C₆alkyleneamino, —NH₂, —NH(C₁-C₆alkyl), —N(C₁-C₆alkyl)₂, —NH(C₁-C₆alkoxy), —C(O)NH₂, —C(O)NHC₁-C₆alkyl, —C(O)N(C₁-C₆alkyl)₂, —COOH, —C₁-C₆alkyleneCOOH, —C₃-C₆cycloalkylCOOH, —C₁-C₆alkyleneC(O)NH₂, —C₃-C₆cycloalkylC(O)NH₂, —C₁-C₆alkyleneC(O)NHC₁-C₆alkyl, —C₁-C₆alkyleneC(O)N(C₁-C₆alkyl)₂, —C(O)C₁-C₆alkyl, —C(O)OC₁-C₆alkyl, —NHC(O)(C₁-C₆alkyl), —N(C₁-C₆alkyl)C(O)(C₁-C₆alkyl), —S(O)C₁-C₆alkyl, —S(O)C₃-C₆cycloalkyl, —S(O)₂C₁-C₆alkyl, —S(O)₂C₃-C₆cycloalkyl, oxo (═O), 3-7-membered heterocycloalkyl, aryl, or heteroaryl groups. In some embodiments, the C₁-C₆alkyl group in any of the substituent groups in this paragraph is a C₁-C₄alkyl; in some embodiments it is C₁-C₃alkyl. In some embodiments, the C₁-C₆alkylene group in any of the substituent groups in this paragraph is a C₁-C₄alkylene. In some embodiments, the C₁-C₆haloalkyl substituent is a C₁-C₄haloalkyl; in some embodiments, it is C₁-C₃haloalkyl. In some embodiments, the C₃-C₆cycloalkyl substituent is a C₃-C₄cycloalkyl substituent. In some embodiments, the C₁-C₆alkoxy substituent is a C₁-C₃alkoxy; in some embodiments, it is C₁-C₄alkoxy. In some embodiments, the C₁-C₆haloalkoxy substituent is a C₁-C₃haloalkoxy; in some embodiments, it is C₁-C₄haloalkoxy.

In some embodiments, “optionally substituted,” refers to the following substituents: halo, CN, NO₂, N₃, alkyl, alkenyl, alkynyl, cycloalkyl, aryl, heterocycloalkyl, heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹⁾₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; wherein each instance of Rⁿ¹ is independently hydrogen, an optionally substituted C₁-C₆alkyl, or a nitrogen protecting group; each instance of R^{o1} is independently hydrogen, an optionally substituted C₁-C₆alkyl, or an oxygen protecting group; and each instance of R^{c1} is an optionally substituted C₁-C₆alkyl; and each instance of R^{s1} is independently an optionally substituted C₁-C₆alkyl or a sulfur protecting group.

As used herein, the term “alkenyl” refers to a straight- or branched-chain group having from 2 to 12 carbon atoms (“C₂-C₁₂”) in the group, wherein the group includes at least one carbon-carbon double bond. Examples of alkenyl groups include vinyl (—CH═CH₂; C₂alkenyl) allyl (—CH₂—CH═CH₂; C₃alkenyl), propenyl (—CH═CHCH₃; C₃alkenyl); isopropenyl (—C(CH₃)═CH₂; C₃alkenyl), butenyl (—CH═CHCH₂CH₃; C₄alkenyl), sec-butenyl (—C(CH₃)═CHCH₃; C₄alkenyl), iso-butenyl (—CH═C(CH₃)₂; C₄alkenyl), 2-butenyl (—CH₂CH═CHCH₃; C₄alkyl), pentenyl (CH≡CHCH₂CH₂CH₃; C₅alkenyl), and the like. In some embodiments, the alkenyl group is a C₂-C₄ alkenyl group.

As used herein, the term “alkynyl” refers to a straight- or branched-chain group having from 2 to 12 carbon atoms (“C₂-C₁₂”) in the group, and wherein the group includes at least one carbon-carbon triple bond. Examples of alkynyl groups include ethynyl (—C≡CH; C₂alkynyl); propargyl (—CH₂—C≡CH; C₃alkynyl), propynyl (—C≡CCH₃; C₃alkynyl); butynyl (—C≡CCH₂CH₃; C₄alkynyl), pentynyl (C≡CCH₂CH₂CH₃; Csalkynyl), and the like. In some embodiments, the alkynyl group is a C₂-C₄ alkynyl group.

As used herein, the term “alkoxy” refers to an oxygen radical attached to an alkyl group by a single bond. Examples of alkoxy groups include methoxy (—OCH₃), ethoxy (—OCH₂CH₃), propoxy (e.g., —OⁿPr, —OⁱPr), butoxy (e.g., —OⁿBu,—OⁱBu, —O^{s}Bu,—O^{t}Bu) and the like. In some embodiments, the alkoxy group is a C₁-C₆alkoxy; in some embodiments, it is a C₁-C₄alkoxy; and in some embodiments, it is a C₁-C₃alkoxy.

As used herein, the term “deuterated alkoxy” refers to an alkoxy group as defined herein in which one or more hydrogen atoms has been replaced by deuterium. Examples of deuterated alkoxy groups include—OCD₃, —OCH₂CD₃, and the like. In some embodiments, the deuterated alkoxy group is a deuterated C₁-C₆alkoxy; in some embodiments, it is a deuterated C₁-C₄alkoxy; and in some embodiments, it is a deuterated C₁-C₃alkoxy.

As used herein, the term “alkoxy(alkylene)” as used by itself or as part of a substituent group refers to an alkylene group as defined herein that is bound to an alkoxy group as defined herein. Examples of alkoxy(alkylene) groups include —CH₂OCH₃, —CH₂CH₂OCH₃, and the like.

As used herein, the term “haloalkyl” refers to an alkyl group wherein one or more of the hydrogen atoms has been replaced with one or more halogen atoms which may be the same or different. In some embodiments, the alkyl is substituted by at least one halogen. In some embodiments, the alkyl is substituted by one, two, or three F and/or Cl. Examples of haloalkyl groups include fluoromethyl (CH₂F), 1-fluoroethyl (CH(CH₃)F), 2-fluoroethyl, difluoromethyl (CHF₂), trifluoromethyl (CF₃), pentafluoroethyl, 1,1-difluoroethyl (C(CH₃)F₂), 2,2-difluoroethyl (CH₂CHF₂), 2,2,2-trifluoroethyl (CH₂CF₃), 2-fluoropropan-2-yl (C(CH₃)₂F), 3,3,3-trifluoropropyl, 4,4,4-trifluorobutyl, trichloromethyl and the like. In some embodiments, the haloalkyl group is a C₁-C₆haloalkyl; in some embodiments, it is a C₁-C₄haloalkyl; and in some embodiments, it is a C₁-C₃haloalkyl.

As used herein, the term “haloalkoxy” refers to an oxygen radical attached to a haloalkyl group by a single bond, wherein haloalkyl is defined above. Examples of haloalkoxy groups include fluoromethoxy (OCH₂F), 2-fluoroethoxy, difluoromethoxy (OCHF₂), trifluoromethoxy (OCF₃), pentafluoroethoxy, 1,1-difluoroethoxy (OC(CH₃)F₂), 2,2-difluoroethoxy, 2,2,2-trifluoroethoxy (OCH₂CF₃), 3,3,3-trifluoropropoxy, (1,1,1,3,3,3-hexafluoropropan-2-yl)oxy (OCH(CF₃)₂), 4,4,4-trifluorobutoxy, trichloromethoxy groups, and the like. In some embodiments, the haloalkoxy group is C₁-C₆haloalkoxy; in some embodiments, it is C₁-C₄haloalkoxy; and in some embodiments, it is C₁-C₃haloalkoxy.

As used herein, the term “hydroxyalkyl” refers to an alkyl group wherein one or more of the hydrogen atoms has been replaced with one or more hydroxyl (i.e., —OH) groups. In some embodiments, the hydroxyalkyl contains one OH. In some embodiments, the hydroxyalkyl contains two OH. In further embodiments, the hydroxyalkyl contains three OH. Examples of hydroxyalkyl groups include hydroxymethyl, hydroxyethyl (e.g., 1-hydroxyethyl, 2-hydroxyethyl), 1,2-dihydroxyethyl, hydroxypropyl (e.g., 2-hydroxypropyl, 3-hydroxypropyl), hydroxybutyl (e.g., 3-hydroxybutyl, 4-hydroxybutyl), 2-hydroxy-1-methylpropyl, 1,3 dihydroxyprop-2-yl, and the like. In some embodiments, the hydroxyalkyl group is C₁-C₆hydoxyalkyl; in some embodiments, it is C₁-C₄hydroxyalkyl; and in some embodiments, it is C₁-C₃hydroxyalkyl.

As used herein, the term “cyanoalkyl” as used by itself or as part of a substituent group refers to an alkyl group wherein one or more of the hydrogen atoms has been replaced by one or cyano (i.e., —CN) groups. In some embodiments, the cyanoalkyl contains one cyano. In some embodiments, the cyanoalkyl contains two cyano. In further embodiments, the cyanoalkyl contains three cyano. Examples of cyanoalkyl groups include CH₂CN, CH₂CH₂CN, CH(CN)CH₃, CH₂CH₂CH₂CN, C(CH₃)₂CN, CH₂CH(CN)CH₃, CH(CN)CH₂CH₃, and the like. In some embodiments, the cyanoalkyl group is a C₁₋₆cyanoalkyl; in some embodiments, it is C₁₋₄cyanoalkyl; and in some embodiments, it is C₁₋₃cyanoalkyl.

As used herein, the term “imidazolidinonyl” refers to a five-membered saturated ring radical containing two-nitrogen atoms and three-carbon atoms wherein one of the carbon atoms is oxo-substituted (═O). Non-limiting examples include:

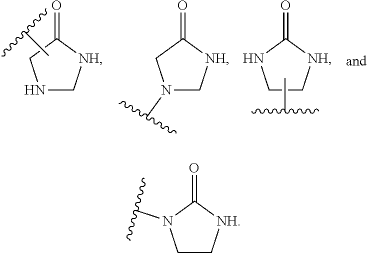

As used herein, the term “pyrrolidinonyl” refers to a five-membered saturated ring radical containing one nitrogen atom and four carbon atoms wherein one of the carbon atoms is oxo-substituted (═O). Non-limiting examples include:

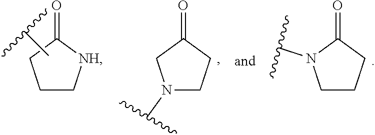

As used herein, the term “dihydropyrazolonyl” refers to a five-membered ring radical containing one unsaturation, two adjacent nitrogen atoms, and three carbon atoms wherein one of the carbon atoms is oxo-substituted (═O). Non-limiting examples include:

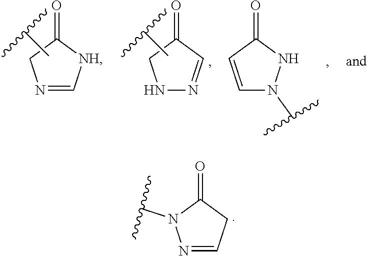

As used herein, the term “dihydropyrrolonyl” refers to a five-membered ring radical containing one unsaturation, one nitrogen atom, and four carbon atoms wherein one of the carbon atoms is oxo-substituted (═O). Non-limiting examples include:

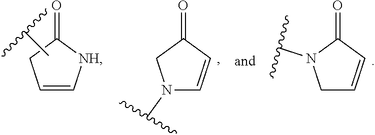

As used herein, the term “dihydroimidazolonyl” refers to a five-membered ring radical containing one unsaturation, two non-adjacent nitrogen atoms, and three carbon atoms wherein one of the carbon atoms is oxo-substituted (═O). Non-limiting examples include:

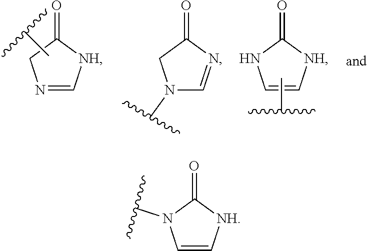

As used herein, the term “dihydrotriazolonyl” refers to a five-membered ring radical containing one unsaturation, three nitrogen atoms, and two carbon atoms wherein one of the carbon atoms is oxo-substituted (═O). Non-limiting examples include:

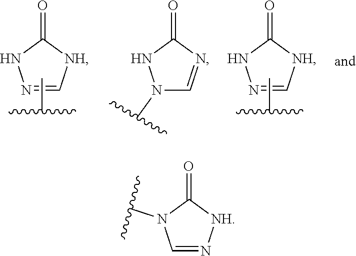

The term “nitrogen protecting group” refers to a moiety that is attached to a nitrogen atom to prevent reaction at that nitrogen atom. Nitrogen protecting groups will be known by those skilled in the art and include those described in Wuts, P. G., Greene's Protective Groups in Organic Synthesis, Wiley; 5th edition (Oct. 27, 2014), which is incorporated by reference herein.

The term “oxygen protecting group” refers to a moiety that is attached to an oxygen atom to prevent reaction at that oxygen atom. Oxygen protecting groups will be known by those skilled in the art and include those described in Wuts, P. G., Greene's Protective Groups in Organic Synthesis. Wiley; 5th edition (Oct. 27, 2014), which is incorporated by reference herein.

The term “sulfur protecting group” refers to a moiety that is attached to a sulfur atom to prevent reaction at that sulfur atom. Sulfur protecting groups will be known by those skilled in the art and include those described in Wuts, P. G., Greene's Protective Groups in Organic Synthesis. Wiley; 5th edition (Oct. 27, 2014), which is incorporated by reference herein,

Recitation of ranges of values herein are intended to serve as a shorthand method of referring individually to each separate value falling within the range, unless otherwise indicated herein, and each separate value is incorporated into the specification as if it were individually recited herein. The use of any and all examples, or exemplary language (e.g., “such as”) provided herein, is intended to better illustrate the disclosure and is not a limitation on the scope of the disclosure unless otherwise claimed. No language in the specification should be construed as indicating any non-claimed element as essential to the practice of the disclosure.

The term “about” when used in combination with a numeric value or range of values means the value or range of values may deviate to an extent deemed reasonable to one of ordinary skill in the art.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including supercritical fluid chromatography (SFC), chiral high pressure liquid chromatography (HPLC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, IN 1972).

Exemplary compounds of the disclosure including a chiral center may be depicted herein as having particular stereochemistries, but for which absolute stereochemistry has not been obtained. Absolute configurations can be obtained using methods known in the art.

As used herein, the term “stereoisomers” refers to compounds which have identical chemical constitution and connectivity but differ with regard to the arrangement of the atoms or groups in space, e.g., enantiomers or diastereomers.

When the stereochemical configuration at a chiral center in a compound having one or more chiral centers is depicted by its chemical name (e.g., where the configuration is indicated in the chemical name by “R” or “S”) or structure (e.g., the configuration is indicated by dashed or wedge bonds), the enrichment of the indicated configuration relative to the opposite configuration is greater than 50%, 60%, 70%, 80%, 90%, 99% or 99.9%. “Enrichment of the indicated configuration relative to the opposite configuration” is a mole percent and is determined by dividing the number of compounds with the indicated stereochemical configuration at the chiral center(s) by the total number of all of the compounds with the same or opposite stereochemical configuration in a mixture.

When a disclosed compound is named or depicted by structure without indicating stereochemistry, it is understood that the name or the structure encompasses one of the possible stereoisomers or geometric isomers free of the others, or a mixture of the encompassed stereoisomers or geometric isomers.

It will be understood that certain compounds disclosed herein may exist in tautomeric forms. Such forms are included as part of the present disclosure. Thus, when a compound herein is represented by a structural formula or designated by a chemical name herein, all tautomeric forms which may exist for the compound are encompassed by the structural formula.

When the compounds described herein contain olefinic double bonds or other centers of geometric asymmetry, and unless specified otherwise, it is intended that they include both E and Z geometric isomers.

In some embodiments, the compounds described herein are isotopically enriched compounds, e.g., an isotopologue. The term “isotopically enriched” refers to an atom having an isotopic composition other than the naturally abundant isotopic composition of that atom. “Isotopically enriched” may also refer to a compound containing at least one atom having an isotopic composition other than the natural isotopic composition of that atom. In an isotopologue, “isotopic enrichment” refers to the percentage of incorporation of an amount of a specific isotope of a given atom in a molecule in the place of that atom's natural isotopic composition. For example, deuterium enrichment of 1% at a given position means that 1% of the molecules in a given sample contain deuterium at the specified position. Because the naturally occurring distribution of deuterium is about 0.0156%, deuterium enrichment at any position in a compound synthesized using non-enriched starting materials is about 0.0156%.

Thus, as used herein, and unless otherwise indicated, the term “isotopic enrichment factor” refers to the ratio of the isotopic composition to the natural isotopic composition of a specified isotope.

With regard to the compounds provided herein, when a particular atom's position is designated as having deuterium or “D” or “²H”, it is understood that the abundance of deuterium at that position is substantially greater than the natural abundance of deuterium, which is about 0.015%. A position designated as having deuterium typically has a minimum isotopic enrichment factor of, in particular embodiments, at least 1000 (15% deuterium incorporation), at least 2000 (30% deuterium incorporation), at least 3000 (45% deuterium incorporation), at least 3500 (52.5% deuterium incorporation), at least 4000 (60% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation) at each designated deuterium atom. The isotopic enrichment and isotopic enrichment factor of the compounds provided herein can be determined using conventional analytical methods known to one of ordinary skill in the art, including mass spectrometry and nuclear magnetic resonance spectroscopy.

The term “patient” or “subject” is used throughout the specification to describe an animal, preferably a human or a domesticated animal, to whom treatment, including prophylactic treatment, with the compounds or compositions according to the present disclosure is provided. For treatment of those conditions or disease states which are specific for a specific animal such as a human patient, the term patient refers to that specific animal, including a domesticated animal such as a dog or cat or a farm animal such as a horse, cow, sheep, etc. In general, in the present disclosure, the term patient refers to a human patient unless otherwise stated or implied from the context of the use of the term.

In some embodiments, a subject is treated with compounds of the disclosure, or a pharmaceutical composition comprising compounds of the disclosure. In certain embodiments, the subject is a human subject, such as a human adult over 18 years old in need of treatment. In further embodiments, the human subject is a human child less than 18 years old in need of treatment.

The terms “therapeutically effective amount” or “effective amount” means an amount or dose of a compound of the disclosure (or a pharmaceutically acceptable salt thereof) sufficient to generally bring about the desired therapeutic benefit in subjects in need of such treatment for the designated disease or disorder. Further, a therapeutically effective amount with respect to a compound of the disclosure means that amount of therapeutic agent alone, or in combination with other therapies, that provides a therapeutic benefit in the treatment or prevention of a disease.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The terms “prevent,” “preventing,” and “prevention” refer to the prevention of the onset, recurrence, or spread of the disease in a subject resulting from the administration of a prophylactic or therapeutic agent.

In some embodiments, the disclosure is directed to compounds of Formula I or Formula II:

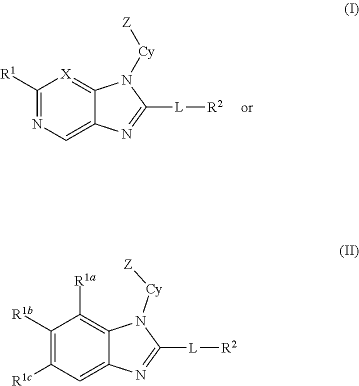

or a pharmaceutically acceptable salt thereof, wherein Z, Cy, L, R¹, R², X, R^{1a}, R^{1b}, and R^{1c} are defined herein.

In some embodiments, the compound is a compound of Formula I or a pharmaceutically acceptable salt thereof,

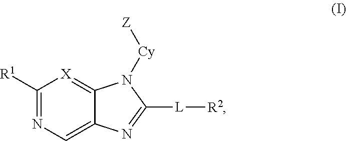

wherein Z, Cy, L, R¹, R², and X are defined herein.

In some embodiments, the compound is a compound of Formula I. In some embodiments, the compound is a pharmaceutically acceptable salt of a compound of Formula I.

In some embodiments, the compound of Formula I is a compound of Formula I-A:

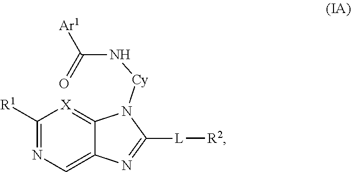

or a pharmaceutically acceptable salt thereof, wherein Ar¹, Cy, L, R¹, R², and X are defined herein.

In some embodiments, the compound of Formula I is a compound of Formula I-B or Formula I-C:

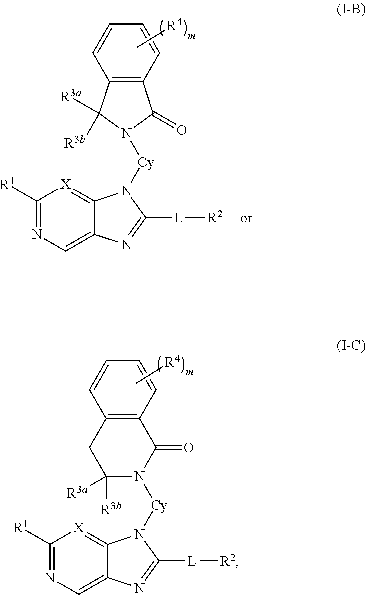

or a pharmaceutically acceptable salt thereof, wherein Cy, L, R¹, R², R^{3a}, R^{3b}, R⁴, m, and X are defined herein.

In some embodiments, the compound of Formula I is a compound of Formula I-B, or a pharmaceutically acceptable salt thereof.

In some embodiments, the compound of Formula I is a compound of Formula I-C, or a pharmaceutically acceptable salt thereof.

In some embodiments, the compound of Formula I is a compound of Formula I-D, Formula I-E, or Formula I-F:

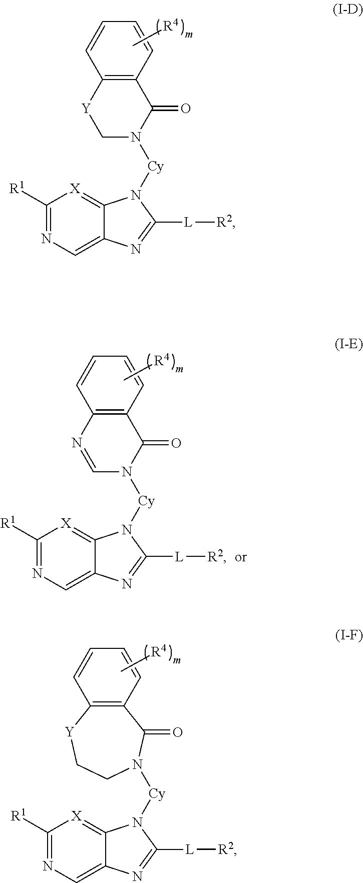

or a pharmaceutically acceptable salt thereof, wherein Cy, L, R¹, R², R⁴, m, X and Y are defined herein.

In some embodiments, the compound of Formula I is a compound of Formula I-D, or a pharmaceutically acceptable salt thereof.

In some embodiments, the compound of Formula I is a compound of Formula I-E, or a pharmaceutically acceptable salt thereof.

In some embodiments, the compound of Formula I is a compound of Formula I-F, or a pharmaceutically acceptable salt thereof.

In some embodiments, the compound is a compound of Formula II or a pharmaceutically acceptable salt thereof,

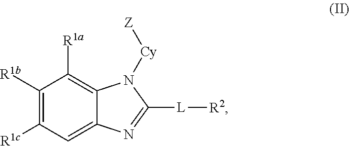

wherein Z, Cy, L, R^{1a}, R^{1b}, R^{1c}, and R² are defined herein.

In some embodiments, the compound is a compound of Formula II. In some embodiments, the compound is a pharmaceutically acceptable salt of a compound of Formula II.

In some embodiments, the compound of Formula II is a compound of Formula II-A:

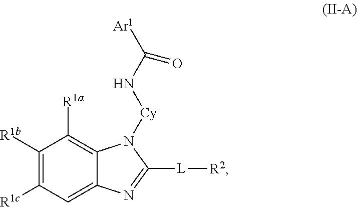

or a pharmaceutically acceptable salt thereof, wherein Ar¹, Cy, L, R^{1a}, R^{1b}, R^{1c}, and R² are defined herein.

In some embodiments, the compound of Formula II is a compound of Formula II-B or Formula II-C:

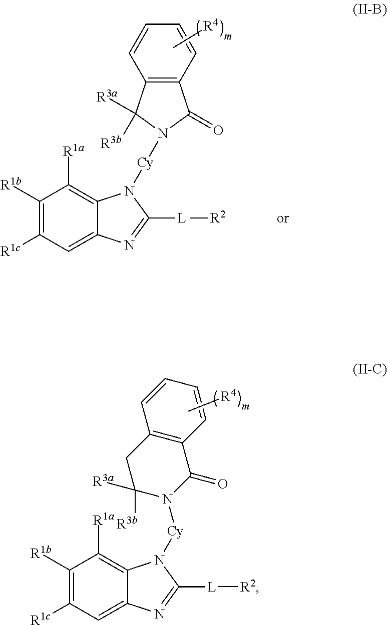

or a pharmaceutically acceptable salt thereof, wherein Cy, L, R^{1a}, R^{1b}, R^{1c}, R², R^{3a}, R^{3b}, R⁴, and m are defined herein.

In some embodiments, the compound of Formula II is a compound of Formula II-B, or a pharmaceutically acceptable salt thereof.

In some embodiments, the compound of Formula II is a compound of Formula II-C, or a pharmaceutically acceptable salt thereof.

In some embodiments, the compound of Formula II is a compound of Formula II-D, Formula II-E, or Formula II-F:

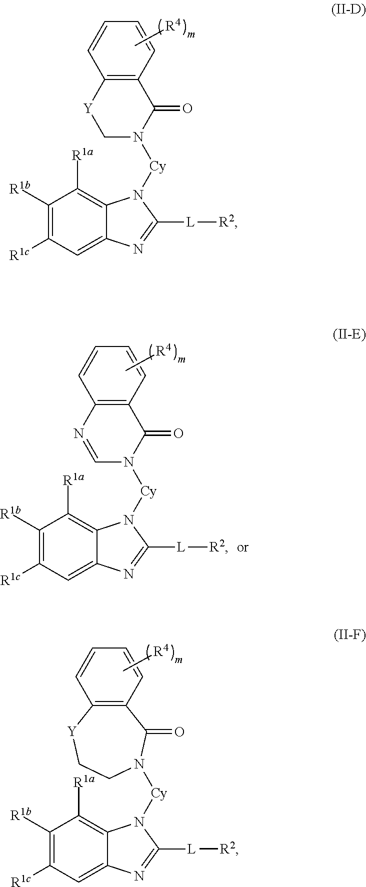

or a pharmaceutically acceptable salt thereof, wherein Cy, L, R^{1a}, R^{1b}, R^{1c}, R², R⁴, m, and Y are defined herein.

In some embodiments, the compound of Formula II is a compound of Formula II-D, or a pharmaceutically acceptable salt thereof.

In some embodiments, the compound of Formula II is a compound of Formula II-E, or a pharmaceutically acceptable salt thereof.

In some embodiments, the compound of Formula II is a compound of Formula II-F, or a pharmaceutically acceptable salt thereof.

In some embodiments, X in the compounds of Formula I, Formula I-A, Formula I-B, Formula I-C, Formula I-D, Formula I-E, or Formula I-F is CH.

In some embodiments, X in the compounds of Formula I, Formula I-A, Formula I-B, Formula I-C, Formula I-D, Formula I-E, or Formula I-F is N.

In some embodiments, Y in the compounds of Formula I, Formula I-D, Formula I-F, Formula II, Formula II-D, or Formula II-F is 0.

In some embodiments, Y in the compounds of Formula I, Formula I-D, Formula I-F, Formula II, Formula II-D, or Formula II-F is N.

In some embodiments, R^{3a} and R^{3b} in the compounds of Formula I, Formula I-B, Formula I-C, Formula II, Formula II-B, or Formula II-C are hydrogen.

In some embodiments, R^{3a} in the compounds of Formula I, Formula I-B, Formula I-C, Formula II, Formula II-B, or Formula II-C is oxo and R^{3b} in the compounds of Formula I, Formula I-B, Formula I-C, Formula II, Formula II-B, or Formula II-C is hydrogen.

In some embodiments, each R⁴ in the compounds of Formula I, Formula I-B, Formula I-C, Formula I-D, Formula I-E, or Formula I-F, Formula II, Formula II-B, Formula II-C, Formula II-D, Formula II-E, or Formula II-F is independently halo.

In some embodiments, each R⁴ in the compounds of Formula I, Formula I-B, Formula I-C, Formula I-D, Formula I-E, Formula I-F, Formula II, Formula II-B, Formula II-C, Formula II-D, Formula II-E, or Formula II-F is independently F or C₁.

In some embodiments, m in the compounds of Formula I, Formula I-B, Formula I-C, Formula I-D, Formula I-E, Formula I-F, Formula II, Formula II-B, Formula II-C, Formula II-D, Formula II-E, or Formula II-F is 1 or 2.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted C₅-C₇ cycloalkyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted C₅-C₇ cycloalkyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is C₅-C₇ cycloalkyl substituted with one or more of OH, deuterium, halo, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}, wherein R^{d} is hydrogen or C₁-C₄alkyl and R^{e} is hydrogen, C₁-C₄alkyl, CO₂(C₁-C₄alkyl), C(O)(C₁-C₄alkyl), SO₂(C₁-C₄alkyl), or SO₂(C₃-C₄cycloalkyl).

In some embodiments, the optionally substituted C₅-C₇cycloalkyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is an optionally substituted cyclohexyl or an optionally substituted cycloheptyl.

In some embodiments, the optionally substituted cyclohexyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

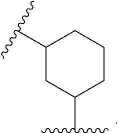 [SMILES: *C1CCCC(*)C1]

In some embodiments, the optionally substituted cyclohexyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

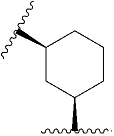 [SMILES: *[C@@H]1CCC[C@H](*)C1]

In some embodiments, the optionally substituted cyclohexyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is substituted with one of OH, C₁-C₃alkoxy, C₁-C₃haloalkoxy or NR^{d}R^{e}, wherein R^{d} is hydrogen or C₁-C₄alkyl and R^{e} is hydrogen or C₁-C₄alkyl.

In some embodiments, the optionally substituted cyclohexyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

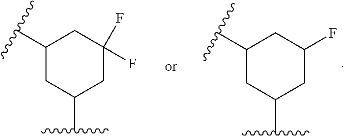 [SMILES: *C1CC(*)CC(F)(F)C1 ; *C1CC(*)CC(F)C1 ; "."]

In some embodiments, the optionally substituted cyclohexyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

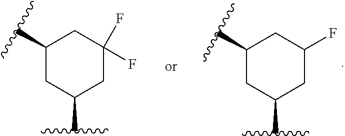 [SMILES: *[C@@H]1C[C@H](*)CC(F)(F)C1 ; *[C@@H]1CC(F)C[C@H](*)C1 ; "."]

In some embodiments, the optionally substituted cycloheptyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

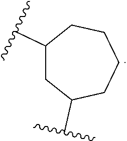 [SMILES: *C1CCCCC(*)C1]

In some embodiments, the optionally substituted cycloheptyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

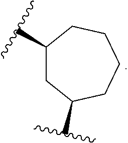 [SMILES: *[C@@H]1CCCC[C@H](*)C1]

In some embodiments, the optionally substituted cycloheptyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is substituted with one of OH or C₁-C₃alkoxy.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted 6-membered heterocycloalkyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted piperidinyl, optionally substituted tetrahydropyranyl, optionally substituted thianyl, optionally substituted thianyl sulfone, optionally substituted thianyl sulfoxide, or optionally substituted 1-oxo-1-imino-1-thiacyclohexyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted piperidinyl.

In some embodiments, the nitrogen in the piperidinyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is substituted with C₁-C₆alkyl, C(O)(C₁-C₆alkyl), oxo, OH, C₁-C₆alkoxy, CO₂(C₁-C₆alkyl), SO₂(C₁-C₆alkyl), or SO₂(C₃-C₆cycloalkyl).

In some embodiments, the nitrogen in the piperidinyl in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is substituted with C₁-C₄alkyl or C(O)(C₁-C₄alkyl).

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted 7-membered heterocycloalkyl, such as optionally substituted oxazepanyl, optionally substituted thiepanyl, optionally substituted thiepanyl-1-oxide, optionally substituted thiepanyl-1,1-dioxide, optionally substituted azepanyl, or optionally substituted 1-imino-1-oxide-thiepanyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is optionally substituted C₅-C₇ cycloalkyl or optionally substituted 6- or 7-membered heterocycloalkyl. In other embodiments, Cy is optionally substituted C₆-C₇ cycloalkyl or optionally substituted 6-membered heterocycloalkyl. In yet other embodiments, Cy is optionally substituted C₆ cycloalkyl or optionally substituted piperidinyl.

It will be understood that the Cy group in the compounds of the disclosure, when unsubstituted, is a diradical with two attachments to other parts of the molecule. As such, the potential exists for Cy to have multiple stereochemical configurations. When no stereochemistry is indicated, such as, for example:

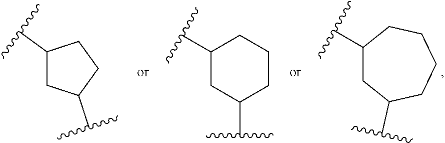 [SMILES: *C1CCC(*)C1 ; *C1CCCC(*)C1 ; *C1CCCCC(*)C1]

the structure is meant to represent all stereoisomeric configurations (e.g., cis and trans) including any mixture of stereoisomers in any proportions. Where a specific stereochemistry is indicated in the structure, then the structure is meant to encompass the stereoisomer with the indicated absolute configuration, but that structure may also contain a mixture of the other enantiomer of the indicated absolute configuration in any ratio. Thus, for example, unless otherwise indicated, the structure

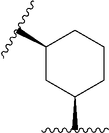 [SMILES: *[C@@H]1CCC[C@H](*)C1]

is meant to represent

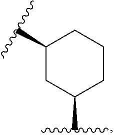 [SMILES: *[C@@H]1CCC[C@H](*)C1]

and may include the enantiomer

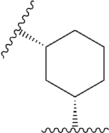 [SMILES: *[C@@H]1CCC[C@H](*)C1]

in any ratio.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted cyclopentyl, such as, for example,

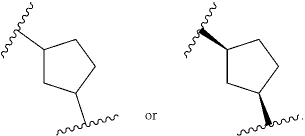 [SMILES: *C1CCC(*)C1 ; *[C@@H]1CC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted cyclohexyl, such as, for example,

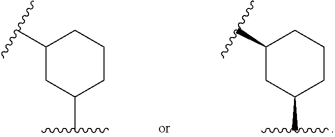 [SMILES: *C1CCCC(*)C1 ; *[C@@H]1CCC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is substituted cyclohexyl.

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

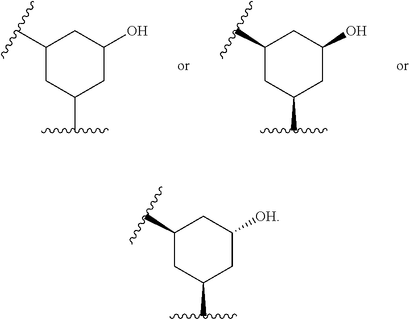 [SMILES: *C1CC(*)CC(O)C1 ; *[C@@H]1C[C@H](*)C[C@H](O)C1 ; *[C@@H]1C[C@H](*)C[C@@H](O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

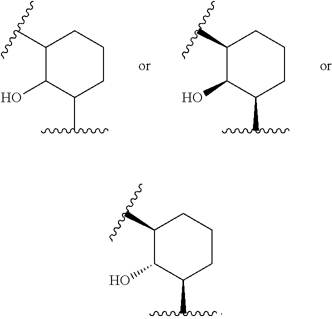 [SMILES: *C1CCCC(*)C1O ; *[C@@H]1CCC[C@H](*)[C@@H]1O ; *[C@@H]1CCC[C@H](*)[C@H]1O]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

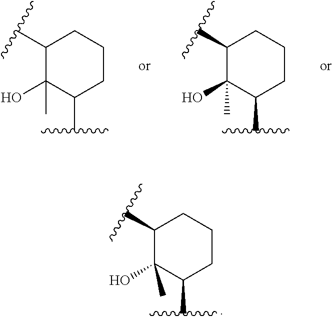

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

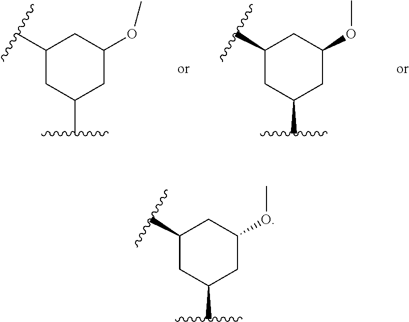

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

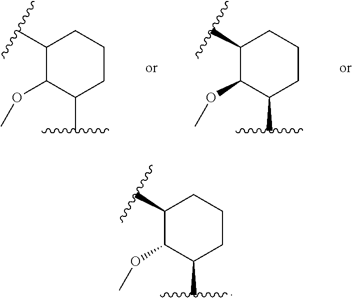

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

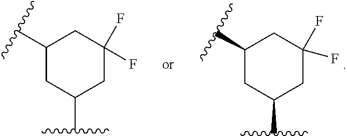 [SMILES: *C1CC(*)CC(F)(F)C1 ; *[C@@H]1C[C@H](*)CC(F)(F)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

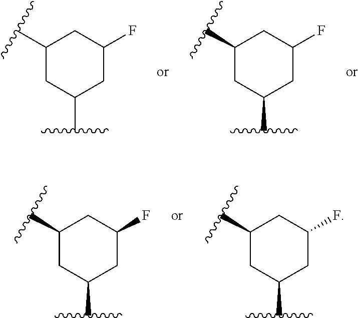 [SMILES: *C1CC(*)CC(F)C1 ; *[C@@H]1CC(F)C[C@H](*)C1 ; *[C@@H]1C[C@H](*)C[C@H](F)C1 ; *[C@@H]1C[C@H](*)C[C@@H](F)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

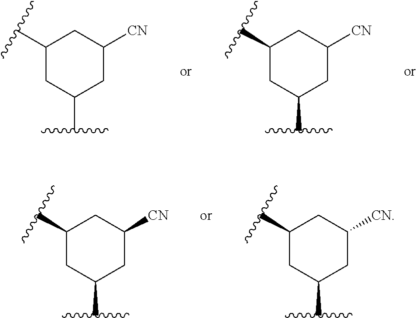 [SMILES: *C1CC(*)CC(C#N)C1 ; *[C@@H]1CC(C#N)C[C@H](*)C1 ; *[C@@H]1C[C@H](*)C[C@H](C#N)C1 ; *[C@@H]1C[C@H](*)C[C@@H](C#N)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

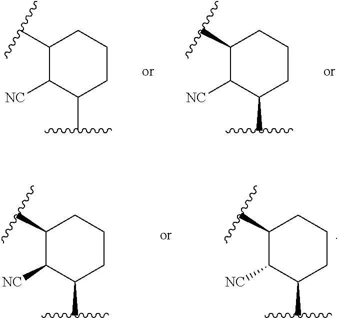 [SMILES: *C1CCCC(*)C1C#N ; *[C@@H]1CCC[C@H](*)C1C#N ; *[C@@H]1CCC[C@H](*)[C@@H]1C#N ; *[C@@H]1CCC[C@H](*)[C@H]1C#N]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

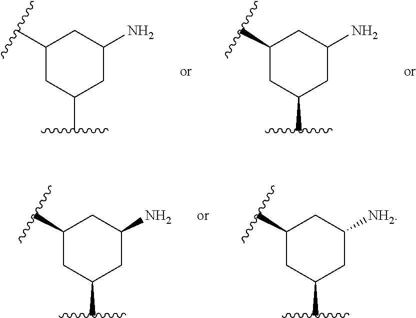 [SMILES: *C1CC(*)CC(N)C1 ; *[C@@H]1CC(N)C[C@H](*)C1 ; *[C@@H]1C[C@H](*)C[C@H](N)C1 ; *[C@@H]1C[C@H](*)C[C@@H](N)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

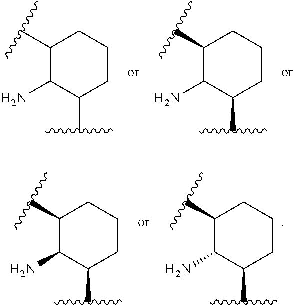 [SMILES: *C1CCCC(*)C1N ; *[C@@H]1CCC[C@H](*)C1N ; *[C@@H]1CCC[C@H](*)[C@@H]1N ; *[C@@H]1CCC[C@H](*)[C@H]1N]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted cycloheptyl, such as, for example,

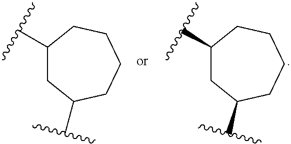 [SMILES: *C1CCCCC(*)C1 ; *[C@@H]1CCCC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is

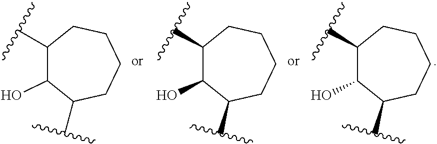 [SMILES: *C1CCCCC(*)C1O ; *[C@@H]1CCCC[C@H](*)[C@@H]1O ; *[C@@H]1CCCC[C@H](*)[C@H]1O]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted piperidinyl, such as, for example,

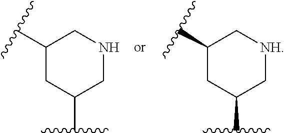 [SMILES: *C1CNCC(*)C1 ; *[C@@H]1CNC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with methyl, such as, for example,

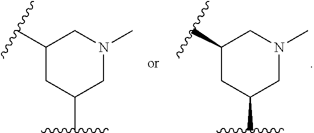

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with ethyl, such as, for example,

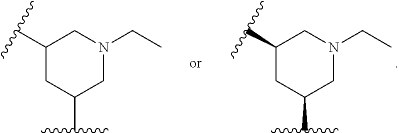

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with acetyl, such as, for example,

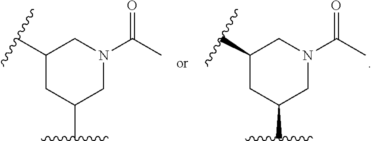 [SMILES: *C1CC(*)CN(C(C)=O)C1 ; *[C@@H]1C[C@H](*)CN(C(C)=O)C1]

In a some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with C₁-C₆hydroxyalkyl, such as, for example,

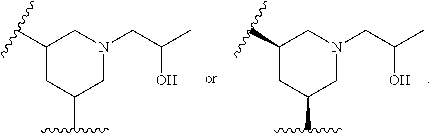 [SMILES: *C1CC(*)CN(CC(C)O)C1 ; *[C@@H]1C[C@H](*)CN(CC(C)O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with C(O)NR^{f}R^{g}, wherein each R^{f} and R^{g} is independently hydrogen or C₁-C₄alkyl, such as, for example,

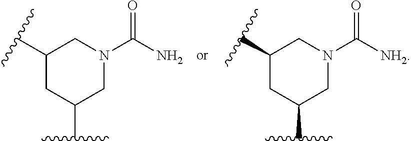 [SMILES: *C1CC(*)CN(C(N)=O)C1 ; *[C@@H]1C[C@H](*)CN(C(N)=O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with SO₂C₁-C₆alkyl, such as, for example,

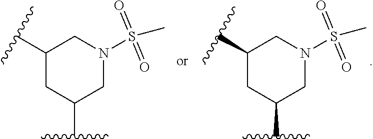 [SMILES: *C1CC(*)CN(S(C)(=O)=O)C1 ; *[C@@H]1C[C@H](*)CN(S(C)(=O)=O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is piperidinyl substituted with C(O)OC₁-C₆alkyl, such as, for example,

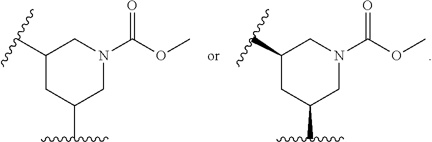 [SMILES: *C1CC(*)CN(C(=O)OC)C1 ; *[C@@H]1C[C@H](*)CN(C(=O)OC)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted tetrahydropyranyl, such as, for example,

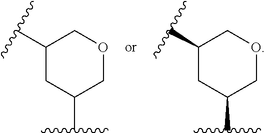 [SMILES: *C1COCC(*)C1 ; *[C@@H]1COC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thianyl, such as, for example,

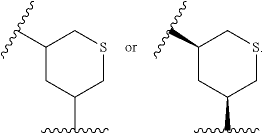 [SMILES: *C1CSCC(*)C1 ; *[C@@H]1CSC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thianyl sulfone, such as, for example,

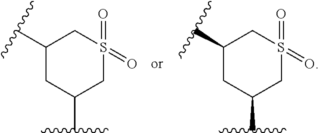 [SMILES: *C1CC(*)CS(=O)(=O)C1 ; *[C@@H]1C[C@H](*)CS(=O)(=O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thianyl sulfoxide, such as, for example,

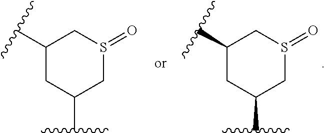 [SMILES: *C1CC(*)CS(=O)C1 ; *[C@@H]1C[C@H](*)CS(=O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted 1-oxo-1-imino-1-thiacyclohexyl, such as, for example,

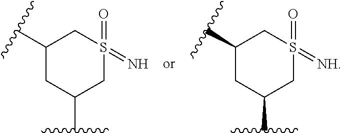 [SMILES: *C1CC(*)CS(=N)(=O)C1 ; *[C@@H]1C[C@H](*)CS(=N)(=O)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted oxazepanyl, such as, for example,

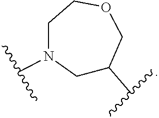 [SMILES: *C1COCCN(*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted oxepanyl, such as, for example,

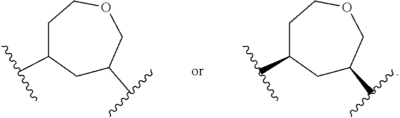 [SMILES: *C1CCOCC(*)C1 ; *[C@@H]1COCC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted dioxepanyl, such as, for example,

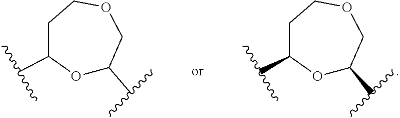 [SMILES: *C1CCOCC(*)O1 ; *[C@@H]1COCC[C@H](*)O1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted azepanyl, such as, for example,

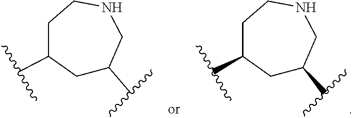 [SMILES: *C1CCNCC(*)C1 ; *[C@@H]1CNCC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted diazepanyl, such as, for example,

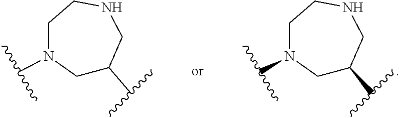

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thiepanyl, such as, for example,

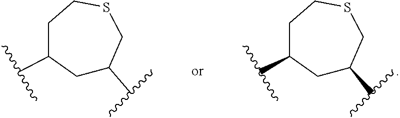 [SMILES: *C1CCSCC(*)C1 ; *[C@@H]1CSCC[C@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thiepanyl-1-oxide, such as, for example,

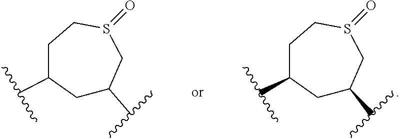 [SMILES: *C1CCS(=O)CC(*)C1 ; *[C@H]1CCS(=O)C[C@@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted thiepanyl-1,1-dioxide, such as, for example,

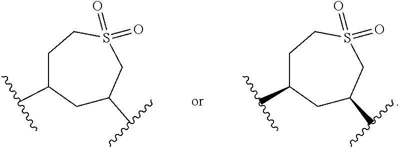 [SMILES: *C1CCS(=O)(=O)CC(*)C1 ; *[C@H]1CCS(=O)(=O)C[C@@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is unsubstituted 1-imino-1-oxide-thiepanyl, such as, for example,

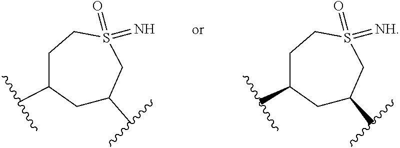 [SMILES: *C1CCS(=N)(=O)CC(*)C1 ; *[C@H]1CCS(=N)(=O)C[C@@H](*)C1]

In some embodiments, Cy in the compounds of Formula I, Formula I-A, Formula I-B, Formula I-C, Formula I-D, Formula I-E, Formula I-F, Formula II, Formula II-A, Formula II-B, Formula II-C, Formula II-D, Formula II-E, or Formula II-F is a substituted 1-imino-1-oxide-thiepanyl.

In some embodiments, the compound of Formula I is a compound of Formula I-G:

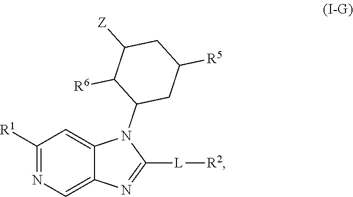

or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}; and
the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl), and L, R¹, R², and Z are defined herein.

In some embodiments, the compound of Formula I-G is a compound of Formula I-G-1:

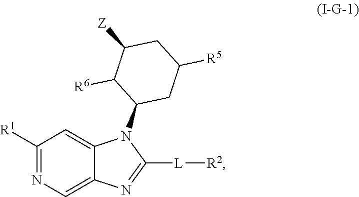

or a pharmaceutically acceptable salt thereof.

In some embodiments, the compound of Formula II is a compound of Formula II-G:

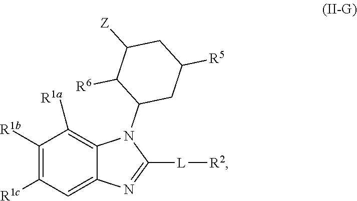

or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}; and
the other of R⁵ or R⁶ is hydrogen or deuterium; and
wherein R^{d} is hydrogen or C₁-C₄alkyl and R^{e} is hydrogen, C₁-C₄alkyl, C₀₂(C₁-C₄alkyl), C(O)C₁-C₄alkyl, SO₂(C₁-C₄alkyl), or SO₂(C₃-C₄cycloalkyl), and L, R^{1a}, R^{1b}, R^{1c}, R², and Z are defined herein.

In some embodiments, the compound of Formula II-G is a compound of Formula II-G-1:

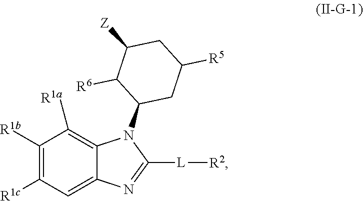

or a pharmaceutically acceptable salt thereof.

In some embodiments, R⁵ in the compounds of Formula I-G, Formula I-G-1, Formula II-G, or Formula II-G-1 is OH, C₁-C₃alkoxy, C₁-C₃haloalkoxy, or NH₂, and R⁶ is hydrogen.

In some embodiments, R⁵ in the compounds of Formula I-G, Formula I-G-1, Formula II-G, or Formula II-G-1 is hydrogen and R⁶ is OH, C₁-C₃alkoxy, C₁-C₃haloalkoxy, or NH₂.

In some embodiments, C₁-C₃alkoxy in the compounds of Formula I-G, Formula I-G-1, Formula II-G, or Formula II-G-1 is OCH₃ and the C₁-C₃haloalkoxy is OCF₃ or OCHF₂.

In some embodiments, the compound of Formula I is a compound of Formula I-H or Formula I-J:

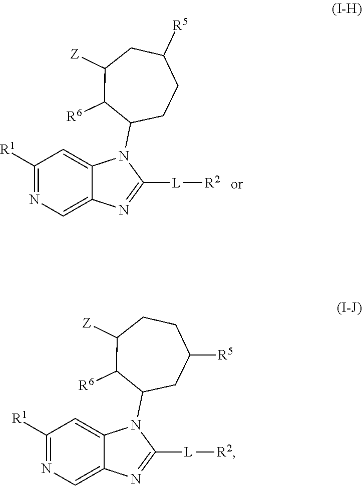

or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO(C₁-C₄alkyl), OSO₂(C₃-C₄cycloalkyl), OC(O)(C₁-C₄alkyl), OC(O)(C₃-C₄cycloalkyl), N₃, or NR^{d}R^{e};
and the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl), and L, R¹, R², and Z are defined herein.

In some embodiments, the compound of Formula I-H is a compound of Formula I-H-1, or a pharmaceutically acceptable salt thereof, and the compound of Formula I-J is a compound of Formula I-J-1, or a pharmaceutically acceptable salt thereof:

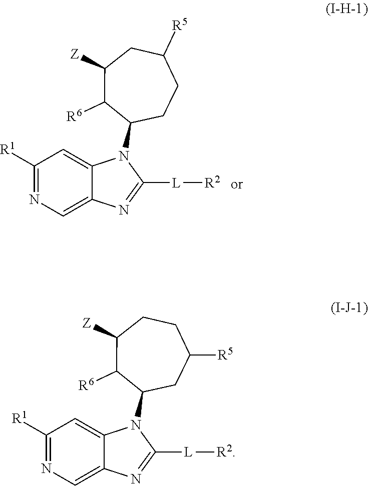

In some embodiments, the compound of Formula II is a compound of Formula II-H or Formula II-J:

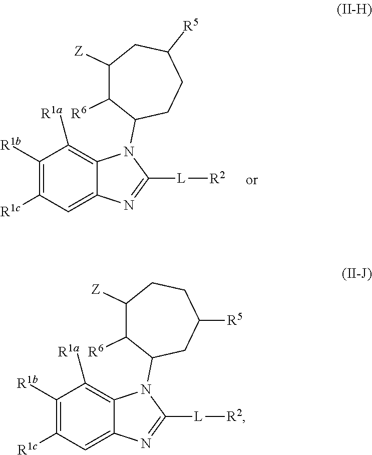

or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e};
and the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl), and L, R^{1a}, R^{1b}, R^{1c}, R², and Z are defined herein.

In some embodiments, the compound of Formula II-H is a compound of Formula II-H-1, or a pharmaceutically acceptable salt thereof, and the compound of Formula II-J is a compound of Formula II-J-1, or a pharmaceutically acceptable salt thereof:

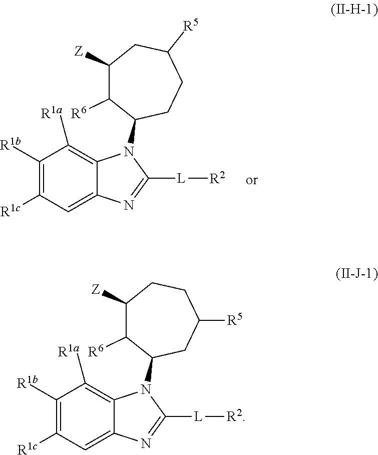

In some embodiments, R⁵ in the compounds of Formula I-H, Formula I-H-1, Formula I-J, Formula I-J-1, Formula II-H, Formula II-H-1, Formula II-J, or Formula II-J-1 is OH or C₁-C₃alkoxy and R⁶ is hydrogen.

In some embodiments, R⁵ in the compounds of Formula I-H, Formula I-H-1, Formula I-J, Formula I-J-1, Formula II-H, Formula II-H-1, Formula II-J, or Formula II-J-1 is hydrogen and R⁶ is OH or C₁-C₃alkoxy.

In some embodiments, the C₁-C₃alkoxy in the compounds of Formula I-H, Formula I-H-1, Formula I-J, Formula I-J-1, Formula II-H, Formula II-H-1, Formula II-J, or Formula II-J-1 is OCH₃.

In some embodiments, the compound of Formula I is a compound of Formula I-K:

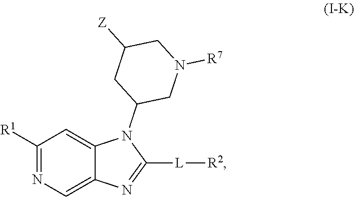

or a pharmaceutically acceptable salt thereof, wherein:
R⁷ is hydrogen, C₁-C₆alkyl, C(O)C₁-C₆alkyl, oxo, OH, C₁-C₆alkoxy, C(O)NR^{f}R^{g}, C₁-C₆hydroxyalkyl, CO₂(C₁-C₆alkyl), SO₂(C₁-C₆alkyl), or SO₂(C₃-C₆cycloalkyl); and
each R^{f} and R^{g} is independently hydrogen or C₁-C₄alkyl, and L, R¹, R², and Z are defined herein.

In some embodiments, the compound of Formula I-K is a compound of Formula I-K-1:

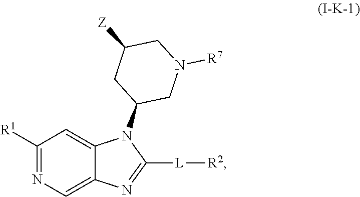

or a pharmaceutically acceptable salt thereof.

In some embodiments, the compound of Formula II is a compound of Formula II-K:

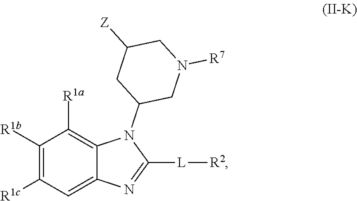

or a pharmaceutically acceptable salt thereof, wherein:
R⁷ is hydrogen, C₁-C₆alkyl, C(O)C₁-C₆alkyl, oxo, OH, C₁-C₆alkoxy, C(O)NR^{f}R^{g}, C₁-C₆hydroxyalkyl, CO₂(C₁-C₆alkyl), SO₂(C₁-C₆alkyl), or SO₂(C₃-C₆cycloalkyl); and
each R^{f} and R^{g} is independently hydrogen or C₁-C₄alkyl, and L, R^{1a}, R^{1b}, R^{1c}, R², and Z are defined herein.

In some embodiments, the compound of Formula II-K is a compound of Formula II-K-1:

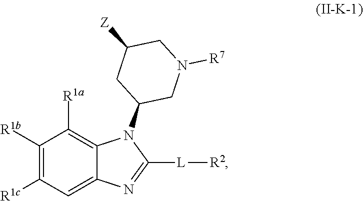

or a pharmaceutically acceptable salt thereof.

In some embodiments, R⁷ in the compounds of Formula I-K, Formula I-K-1, Formula II-K, or Formula II-K-1 is C₁-C₄alkyl or C(O)C₁-C₄alkyl.

In some embodiments, R⁷ in the compounds of Formula I-K, Formula I-K-1, Formula II-K, or Formula II-K-1 is CH₃, CH₂CH₃, or C(O)CH₃.

In some embodiments, Z in the compounds of Formula I, Formula I-G, Formula I-G-1, Formula I-H, Formula I-H-1, Formula I-J, Formula I-J-1, Formula I-K, Formula I-K-1, Formula II, Formula II-G, Formula II-G-1, Formula II-H, Formula II-H-1, Formula II-J, Formula II-J-1, Formula II-K, or Formula II-K-1 is —NHC(O)Ar¹, and Ar¹ is defined herein.

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is optionally substituted 6-membered heteroaryl or optionally substituted phenyl.

In some embodiments, the optionally substituted 6-membered heteroaryl is pyridinyl, pyrimidinyl, or pyridazinyl, each optionally substituted with one or more of halo, C₁-C₆alkyl, C₃-C₆cycloalkyl, C₁-C₆haloalkyl, C₁-C₆alkoxy, C₁-C₆haloalkoxy, C₁-C₆hydroxyalkyl, cyano, OH, oxo, or NR^{j}R^{k}; wherein each R^{j} and R^{k} is independently hydrogen or C₁-C₄alkyl.

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1

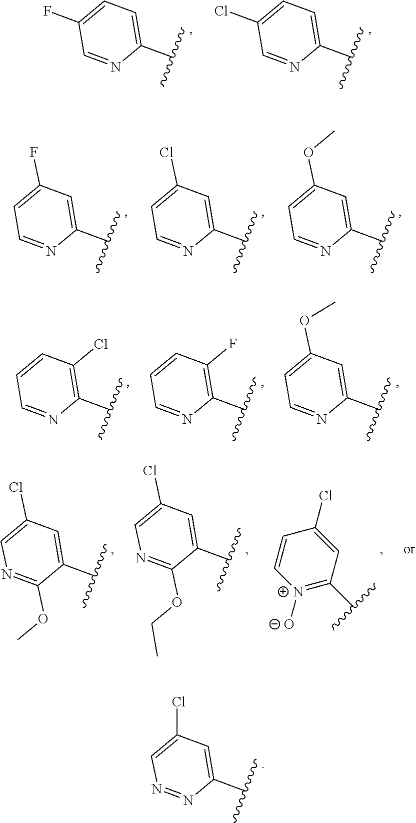

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is

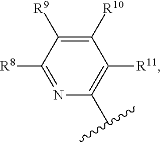

wherein R⁸, R⁹, R¹⁰, and R¹¹ are each independently hydrogen, halo, C₁-C₄alkyl, C₁-C₄haloalkyl, C₁-C₄alkoxy, or C₁-C₄haloalkoxy. In some embodiments, at least two of R⁸, R⁹, R¹⁰, and R¹¹ are hydrogen.

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is phenyl, optionally substituted with one or more of halo, C₁-C₆alkyl, C₃-C₆cycloalkyl, C₁-C₆haloalkyl, C₁-C₆alkoxy, C₁-C₆haloalkoxy, cyano, OH, NR^{j}R^{k}, C(O)NR^{f}R^{g}, CO₂(C₁-C₆alkyl), SO₂(C₁-C₄alkyl), SO₂(C₃-C₄cycloalkyl), C₁-C₄alkylene-R¹², or O—R¹³; wherein R¹² is cyano, NR^{j}R^{k}, OH, or C₁-C₄alkoxy; R¹³ is C₁-C₆hydroxyalkyl, C₃-C₆cycloalkyl, optionally substituted 4-, 5-, or 6-membered heterocycloalkyl, (C₁-C₄alkylene)C₁-C₄alkoxy, (C₁-C₄alkylene)NR^{j}R^{k}, or (C₁-C₄alkylene)-(optionally substituted 4-, 5-, or 6-membered heterocycloalkyl), and each R^{f}, R^{g}, R^{j}, and R^{k} is independently hydrogen or C₁-C₄alkyl.

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is

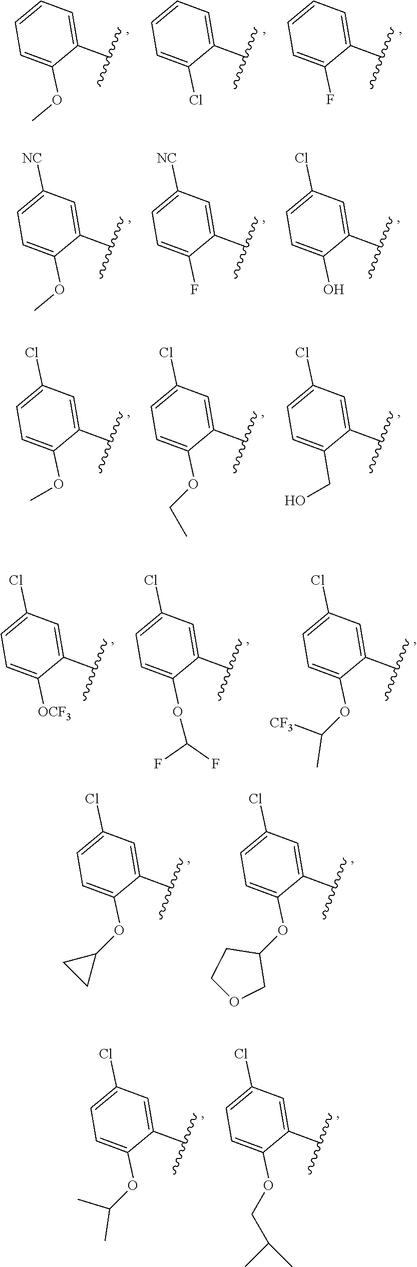

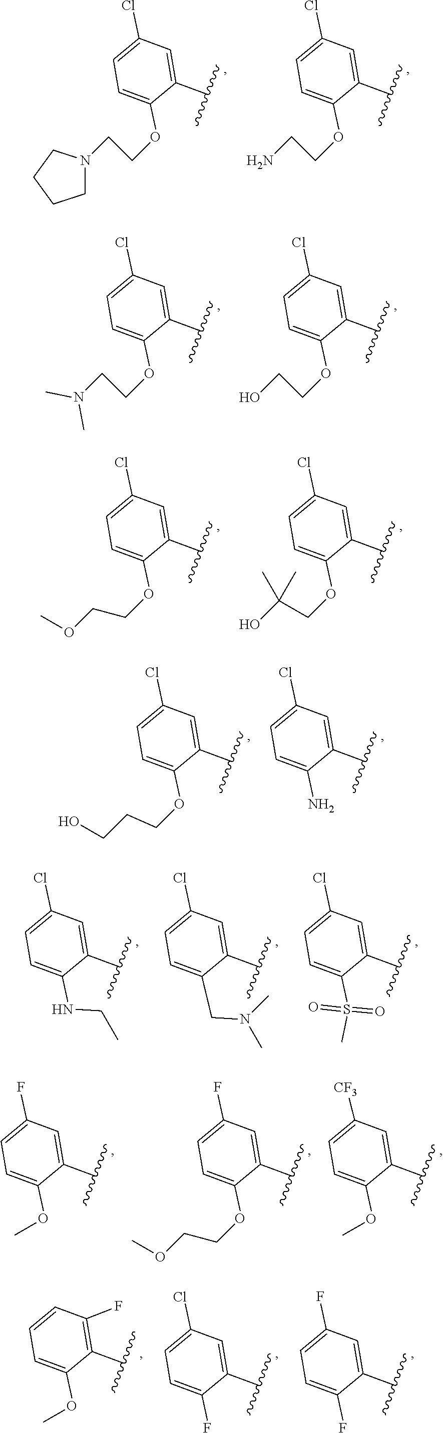

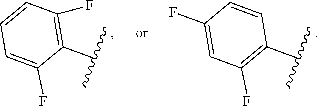 [SMILES: *c1c(F)cccc1F ; *c1ccc(F)cc1F]

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is

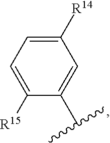

wherein:
R¹⁴ is hydrogen, halo, cyano, or C₁-C₆haloalkyl;
R¹⁵ is halo, C₁-C₆alkoxy, C₁-C₆haloalkoxy, OH, NR^{j}R^{k}, C₁-C₄alkylene-R¹⁶, or O—R¹⁷;
R¹⁶ is NR^{j}R^{k} or OH;
R¹⁷ is C₁-C₆hydroxyalkyl, C₃-C₆cycloalkyl, optionally substituted 4-, 5-, or 6-membered heterocycloalkyl, (C₁-C₄alkylene)C₁-C₄alkoxy, (C₁-C₄alkylene)NR^{j}R^{k}, or (C₁-C₄alkylene)-(optionally substituted 4-, 5-, or 6-membered heterocycloalkyl); and
each R^{j} and R^{k} is independently hydrogen or C₁-C₄alkyl.

In some embodiments, Ar¹ in the compounds of Formula I, Formula I-A, Formula I-G, Formula I-G-1, Formula I-H, Formula I-H-1, Formula I-J, Formula I-J-1, Formula I-K, Formula I-K-1, Formula II, Formula II-A, Formula II-G, Formula II-G-1, Formula II-H, Formula II-H-1, Formula II-J, Formula II-J-1, Formula II-K, or Formula II-K-1 is

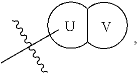

wherein the point of attachment to the carbonyl group of —NHC(O) for variable Z occurs at the phenyl or optionally substituted 6-membered heteroaryl of U, and U and V are defined herein.

In some embodiments, U is optionally substituted 6-membered heteroaryl. In some embodiments, the optionally substituted 6-membered heteroaryl is optionally substituted pyridinyl, optionally substituted pyrimidinyl, or optionally substituted pyridazinyl.

In some embodiments, U is 6-membered heteroaryl, optionally substituted with one or more of halo, cyano, or C₁-C₆haloalkyl.

In some embodiments, U is phenyl substituted with one or more of halo, cyano, or C₁-C₆haloalkyl.

In some embodiments, U is optionally substituted phenyl or optionally substituted 6-membered heteroaryl, and V is optionally substituted 5-membered heterocycloalkyl or optionally substituted 5-membered heteroaryl. In other embodiments, U is optionally substituted phenyl, and V is optionally substituted 5-membered heterocycloalkyl or optionally substituted 5-membered heteroaryl.

In some embodiments, V is optionally substituted furanyl, optionally substituted isofuranyl, optionally substituted oxazolyl, optionally substituted isoxazolyl, optionally substituted thiazolyl, optionally substituted isothiazolyl, optionally substituted pyrrolyl, optionally substituted pyrazolyl, optionally substituted imidazolyl, or optionally substituted thiophenyl. In other embodiments, V is unsubstituted furanyl, unsubstituted isofuranyl, unsubstituted oxazolyl, unsubstituted isoxazolyl, unsubstituted thiazolyl, unsubstituted isothiazolyl, unsubstituted pyrrolyl, unsubstituted pyrazolyl, unsubstituted imidazolyl, or unsubstituted thiophenyl.

In some embodiments, U is phenyl substituted with one or more halo and V is unsubstituted furanyl.

In some embodiments, Z in the compounds of Formula I, Formula I-G, Formula I-G-1, Formula I-H, Formula I-H-1, Formula I-J, Formula I-J-1, Formula I-K, Formula I-K-1, Formula II, Formula II-G, Formula II-G-1, Formula II-H, Formula II-H-1, Formula II-J, Formula II-J-1, Formula II-K, or Formula II-K-1 is

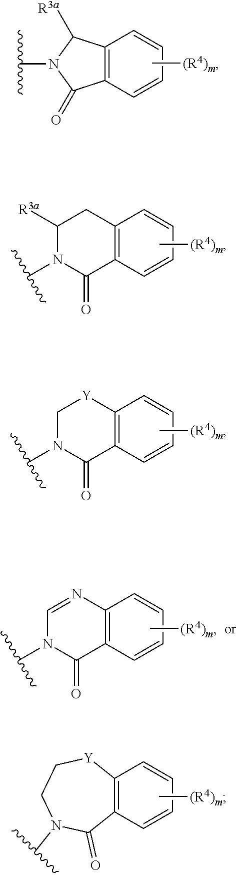

wherein:
R^{3a} is hydrogen or oxo, and Y, R⁴, and m are defined herein.

In some embodiments, each R⁴ in the compounds of Formula I, Formula I-G, Formula I-G-1, Formula I-H, Formula I-H-1, Formula I-J, Formula I-J-1, Formula I-K, Formula I-K-1, Formula II, Formula II-G, Formula II-G-1, Formula II-H, Formula II-H-1, Formula II-J, Formula II-J-1, Formula II-K, or Formula II-K-1 is independently halo and m is 1 or 2.

In some embodiments, each R⁴ in the compounds of Formula I, Formula I-G, Formula I-G-1, Formula I-H, Formula I-H-1, Formula I-J, Formula I-J-1, Formula I-K, Formula I-K-1, Formula II, Formula II-G, Formula II-G-1, Formula II-H, Formula II-H-1, Formula II-J, Formula II-J-1, Formula II-K, or Formula II-K-1 is independently F or C₁.

In some embodiments, Z in the compounds of Formula I, Formula I-G, Formula I-G-1, Formula I-H, Formula I-H-1, Formula I-J, Formula I-J-1, Formula I-K, Formula I-K-1, Formula II, Formula II-G, Formula II-G-1, Formula II-H, Formula II-H-1, Formula II-J, Formula II-J-1, Formula II-K, or Formula II-K-1 is

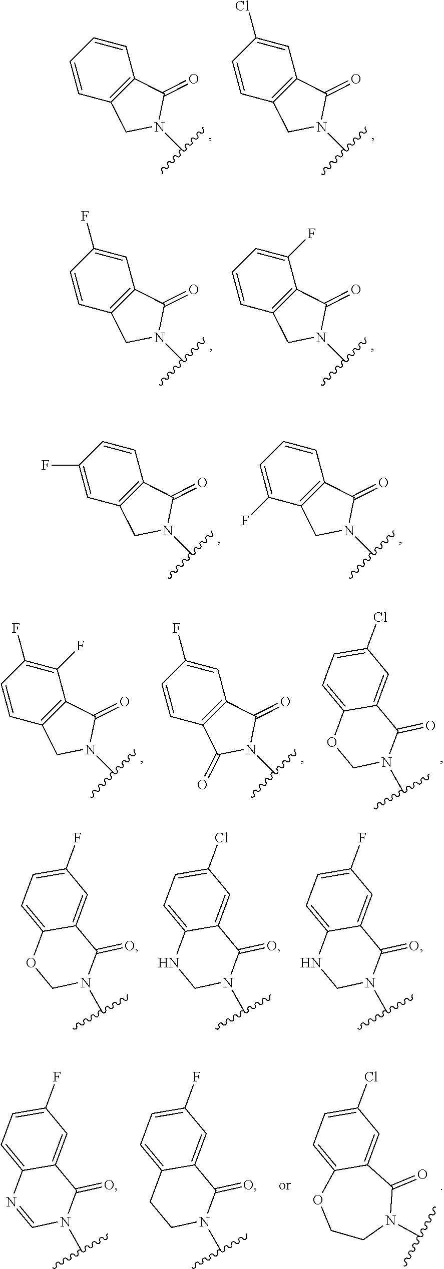

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted heteroaryl.

In some embodiments, R¹ is in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 optionally substituted 5- or 6-membered heteroaryl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted 5-membered heteroaryl. In some embodiments, the optionally substituted 5-membered heteroaryl is optionally substituted oxadiazolyl, optionally substituted thiadiazolyl, optionally substituted oxazolyl, optionally substituted thiazolyl, optionally substituted pyrazolyl, optionally substituted imidazolyl, optionally substituted triazolyl, or optionally substituted tetrazolyl.

In some embodiments, the 5-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, CO₂(C₁-C₆alkyl), or C(O)NR^{f}R^{g}; and wherein each R^{f}, R^{g}, R^{h} and Rⁱ is independently hydrogen or C₁-C₄alkyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted 6-membered heteroaryl. In some embodiments, the optionally substituted 6-membered heteroaryl is optionally substituted pyridinyl, optionally substituted pyrimidinyl, or optionally substituted pyridazinyl.

In some embodiments, the 6-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, CO₂(C₁-C₆alkyl), or C(O)NR^{f}R^{g}; and wherein each R^{f}, R^{g}, R^{h} and Rⁱ is independently hydrogen or C₁-C₄alkyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is

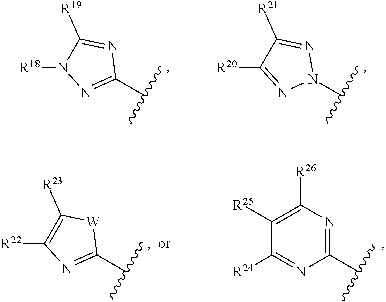

wherein:
R¹⁸ is hydrogen, C₁-C₆alkyl, or C₁-C₆haloalkyl;
R¹⁹ is hydrogen or C₁-C₆alkyl;
R²⁰ and R²¹ are each independently hydrogen, C₁-C₆alkyl, C₁-C₆haloalkyl, C₁-C₆hydroxyalkyl, or CO₂(C₁-C₆alkyl);
W is O or S;
R²² is hydrogen, C₁-C₆alkyl, C₁-C₆haloalkyl, halo, C₁-C₆hydroxyalkyl, or NR^{h}Rⁱ;
R²³ is hydrogen or C₁-C₆alkyl;
R²⁴, R²⁵, and R²⁶ are each independently hydrogen, halo, cyano, C₁-C₆alkyl, C₁-C₆haloalkyl, or C₃-C₆cycloalkyl; and
each R^{h} and Rⁱ is independently hydrogen or C₁-C₄alkyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted aryl. In some embodiments, the optionally substituted aryl is phenyl, optionally substituted with one or more of C₁-C₆alkyl, C₁-C₆haloalkyl, C₁-C₆hydroxyalkyl, halo, cyano, or C₁-C₆cyanoalkyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is optionally substituted 4-, 5- or 6-membered heterocycloalkyl, such as optionally substituted azetidinyl, optionally substituted pyrrolidinyl, optionally substituted tetrahydropyranyl, optionally substituted piperidinyl, optionally substituted morpholinyl, or optionally substituted dioxanyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 is pyrrolidinyl, optionally substituted with one or more of oxo, C₁-C₆hydroxyalkyl, C₁-C₆alkyl, C₁-C₆alkoxy(alkylene), C₁-C₆cyanoalkyl, OH, cyano, C(O)NR^{f}R^{g}, and wherein each R^{f} and R^{g} is independently hydrogen or C₁-C₄alkyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted heteroaryl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 5- or 6-membered heteroaryl. In some embodiments, the optionally substituted 5- or 6-membered heteroaryl is optionally substituted triazolyl, optionally substituted oxadiazolyl, optionally substituted thiadiazolyl, optionally substituted oxazolyl, optionally substituted thiazolyl, optionally substituted pyrazolyl, optionally substituted imidazolyl, optionally substituted tetrazolyl, optionally substituted pyridinyl, optionally substituted pyrimidinyl, or optionally substituted pyridazinyl.

In some embodiments, the optionally substituted 5- or 6-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, C₀₂(C₁-C₆alkyl), or C(O)NR^{f}R^{g}; and wherein each R^{f}, R^{g}, R^{h} and Rⁱ is independently hydrogen or C₁-C₄alkyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is

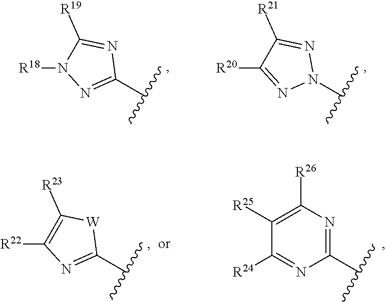

wherein:
R¹⁸ is hydrogen, C₁-C₆alkyl, or C₁-C₆haloalkyl;
R¹⁹ is hydrogen or C₁-C₆alkyl;
R²⁰ and R²¹ are each independently hydrogen, C₁-C₆alkyl, C₁-C₆haloalkyl, C₁-C₆hydroxyalkyl, or C₀₂(C₁-C₆alkyl);
W is O or S;
R²² is hydrogen, C₁-C₆alkyl, C₁-C₆haloalkyl, halo, C₁-C₆hydroxyalkyl, or NR^{h}Rⁱ;
R²³ is hydrogen or C₁-C₆alkyl;
R²⁴, R²⁵, and R²⁶ are each independently hydrogen, halo, cyano, C₁-C₆alkyl, C₁-C₆haloalkyl, or C₃-C₆cycloalkyl; and
each R^{h} and Rⁱ is independently hydrogen or C₁-C₄alkyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted aryl. In some embodiments, the optionally substituted aryl is phenyl, optionally substituted with one or more of C₁-C₆alkyl, C₁-C₆haloalkyl, C₁-C₆hydroxyalkyl, halo, cyano, or C₁-C₆cyanoalkyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 4-, 5- or 6-membered heterocycloalkyl, such as optionally substituted azetidinyl, optionally substituted pyrrolidinyl, optionally substituted tetrahydropyranyl, optionally substituted piperidinyl, optionally substituted morpholinyl.

In some embodiments, R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is pyrrolidinyl, optionally substituted with one or more of oxo, C₁-C₆hydroxyalkyl, C₁-C₆alkyl, C₁-C₆alkoxy(alkylene), C₁-C₆cyanoalkyl, OH, cyano, C(O)NR^{f}R^{g}, and wherein each R^{f} and R^{g} is independently hydrogen or C₁-C₄alkyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

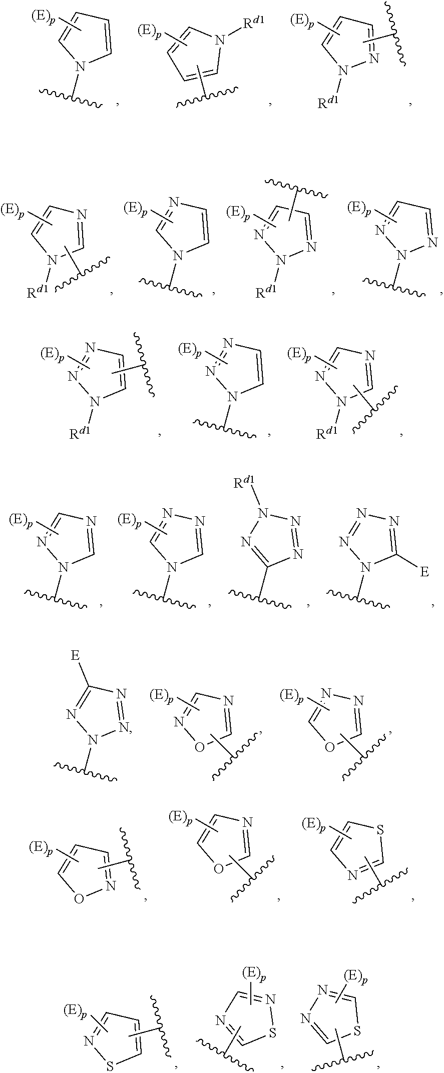

wherein:
each instance of R^{d1} is independently hydrogen, optionally substituted C₁-C₆alkyl, or a nitrogen protecting group;
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, S(═O)(C₃-C₆cycloalkyl), S(═O)₂(C₃-C₆cycloalkyl), N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)^{p} attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R¹ is independently an optionally substituted C₁-C₆alkyl; and
   each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

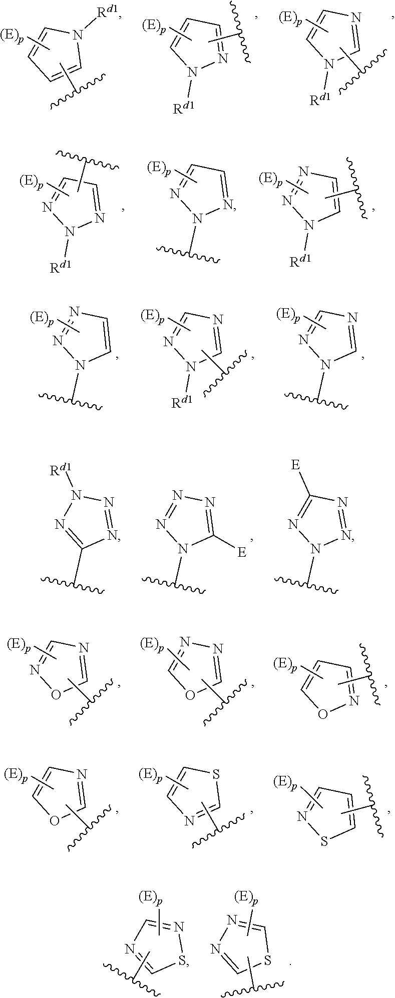

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

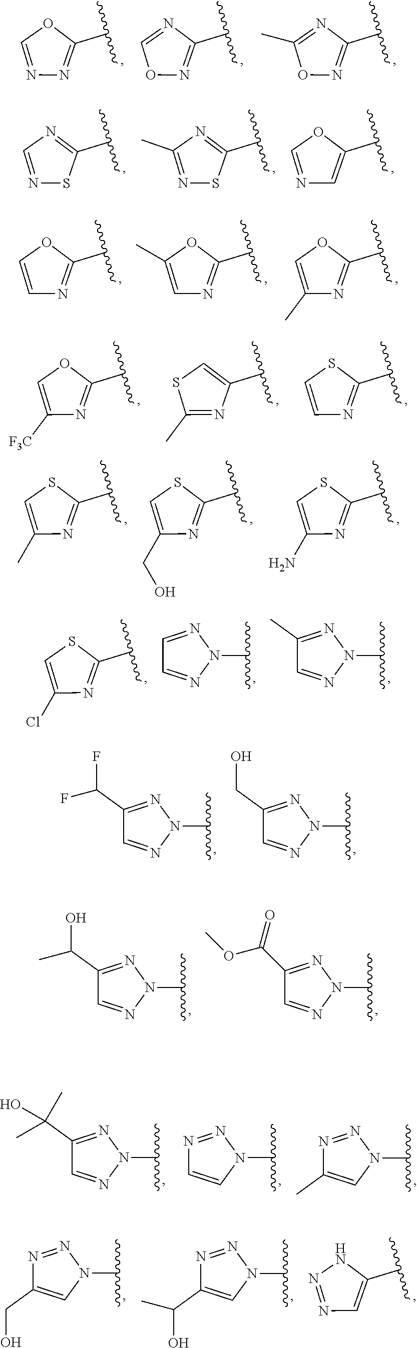

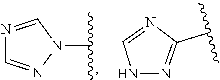

or its tautomer

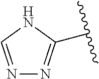 [SMILES: *c1nnc[nH]1]

or its tautomer

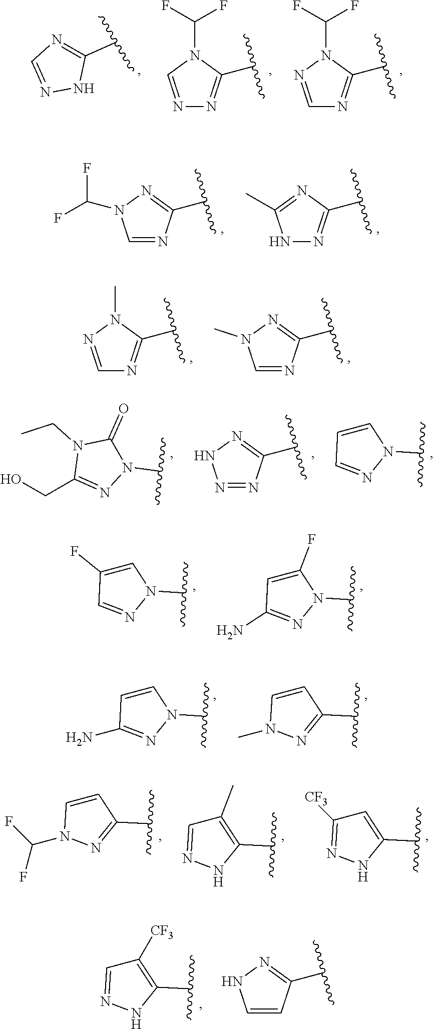

or its tautomer

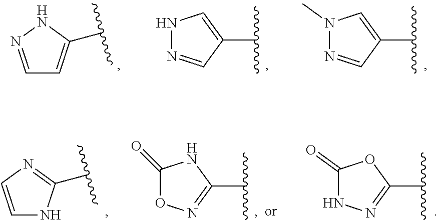

In other embodiments, R¹ and R^{1b} are

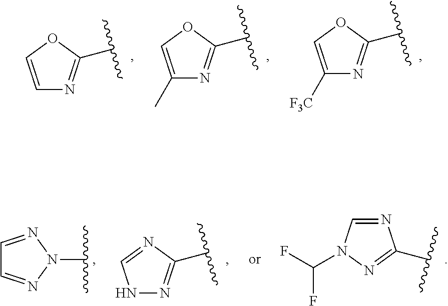

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

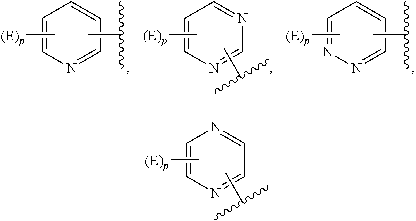

wherein:
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)Rsl, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)p attached to adjacent carbon atoms, can be taken together with the carbon atom(s) to which they are attached to form an optionally substituted cycloalkyl or a heterocycloalkyl; wherein:
   each instance of Rⁿ¹ is independently hydrogen, an optionally substituted C₁-C₆alkyl, or a nitrogen protecting group;
   each instance of R^{o1} is independently hydrogen, an optionally substituted C₁-C₆alkyl, or an oxygen protecting group; and
   each instance of R^{c1} is independently an optionally substituted C₁-C₆alkyl; and
   each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

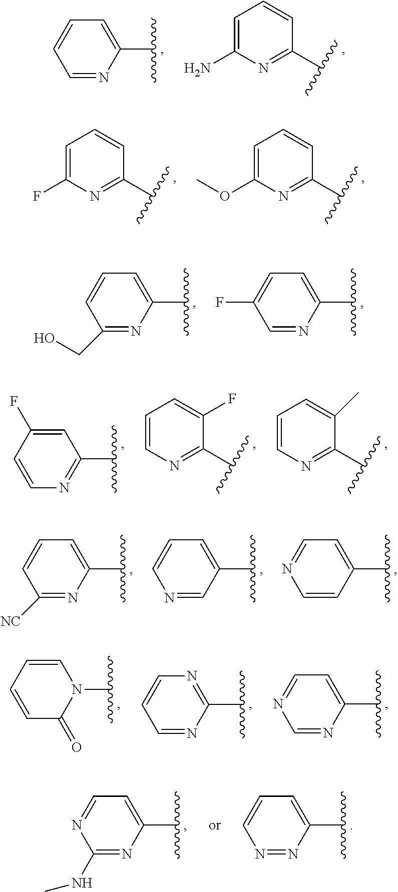

In other embodiments, R¹ and R^{1b} are

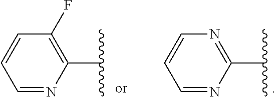 [SMILES: *c1ncccc1F ; *c1ncccn1]

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

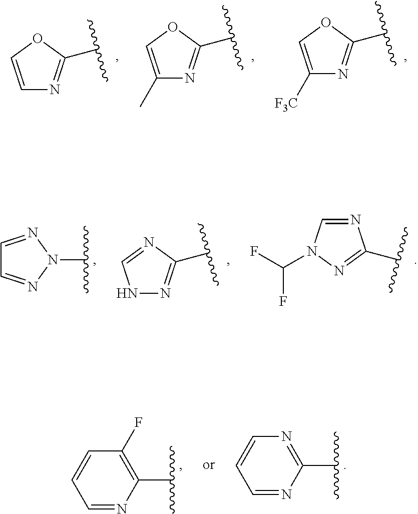

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

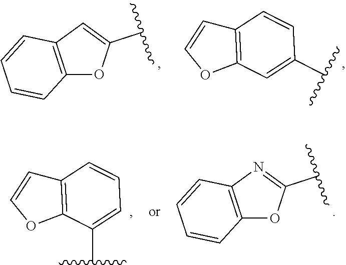

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

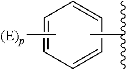

wherein:
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)ₚ attached to adjacent carbon atoms, can be taken together with the carbon atom(s) to which they are attached to form an optionally substituted cycloalkyl or a heterocycloalkyl; wherein:
each instance of Rⁿ¹ is independently hydrogen, an optionally substituted C₁-C₆alkyl, or a nitrogen protecting group;
each instance of R^{o1} is independently hydrogen, an optionally substituted C₁-C₆alkyl, or an oxygen protecting group; and
each instance of R^{c1} is independently an optionally substituted C₁-C₆alkyl; and
each instance of R^{s1} is independently an optionally substituted C₁-C₆alkyl or a sulfur protecting group; and
p is 0, 1, 2, 3, 4, or 5, as valency permits. Where a particular atom is not substituted with E, it should be understood as having the required number of hydrogens as valency permits so as to form a stable ring.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

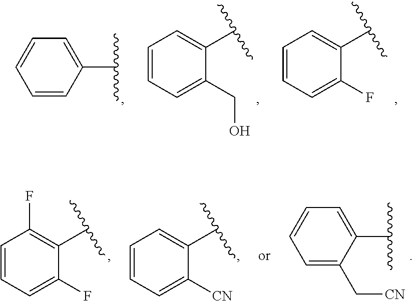

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are

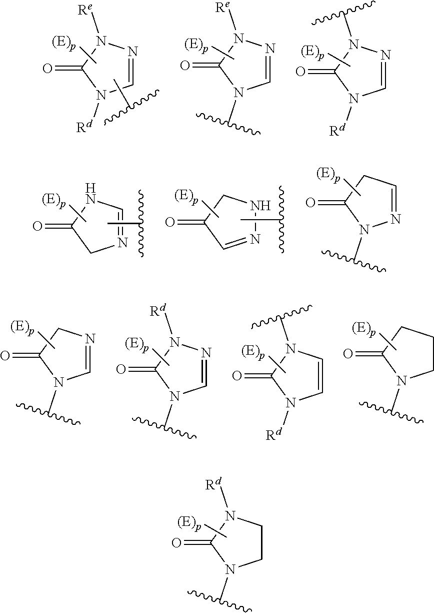

wherein:
each instance of R^{d} and R^{e} is independently hydrogen, optionally substituted C₁-C₆alkyl, or a nitrogen protecting group;
each instance of E is independently halo, CN, NO₂, N₃, an optionally substituted alkyl, an optionally substituted alkenyl, an optionally substituted alkynyl, an optionally substituted cycloalkyl, an optionally substituted aryl, an optionally substituted heterocycloalkyl, an optionally substituted heteroaryl, OR^{o1}, SR^{s1}, N(Rⁿ¹)₂, C(═O)N(Rⁿ¹)₂, N(Rⁿ¹)C(═O)R^{c1}, C(═O)R^{c1}, C(═O)OR^{o1}, OC(═O)R^{c1}, S(═O)R^{s1}, S(═O)₂R^{s1}, S(═O)(C₃-C₆cycloalkyl), S(═O)₂(C₃-C₆cycloalkyl), S(═O)OR^{o1}, OS(═O)R^{c1}, S(═O)₂OR^{o1}, OS(═O)₂R^{c1}, S(═O)N(Rⁿ¹)₂, S(═O)₂N(Rⁿ¹)₂, N(Rⁿ¹)S(═O)R^{s1}, N(Rⁿ¹)S(═O)₂R^{s1}, N(Rⁿ¹)C(═O)OR^{o1}, OC(═O)N(Rⁿ¹)₂, N(Rⁿ¹)C(═O)N(Rⁿ¹)₂, N(Rⁿ¹)S(═O)N(Rⁿ¹)₂, N(Rⁿ¹)S(═O)₂N(Rⁿ¹)₂, N(Rⁿ¹)S(═O)OR^{o1}, N(Rⁿ¹)S(═O)₂OR^{o1}, OS(═O)N(Rⁿ¹)₂, or OS(═O)₂N(Rⁿ¹)₂; or alternatively two instances of (E)p attached to the same or adjacent carbon atoms, can be taken together with the carbon atom(s) to which they are attached to form an optionally substituted cycloalkyl or a heterocycloalkyl; wherein:
each instance of Rⁿ¹ is independently hydrogen, an optionally substituted C₁-C₆alkyl, or a nitrogen protecting group;
each instance of R^{o1} is independently hydrogen, an optionally substituted C₁-C₆alkyl, or an oxygen protecting group; and
each instance of R^{c1} is independently an optionally substituted C₁-C₆alkyl; and
each instance of R^{s1} is independently an optionally substituted C₁-C₆alkyl or a sulfur protecting group; and
p is 0, 1, 2, 3, or 4, as valency permits. Where a particular atom is not substituted with E, it should be understood as having the required number of hydrogens as valency permits so as to form a stable ring.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are optionally substituted 4-, 5-, or 6-membered heterocycloalkyl, such as, for example, optionally substituted: azetidinyl, pyrrolidinyl, dioxolanyl, imidazolidinyl, pyrazolidinyl, piperazinyl, piperidinyl, dioxanyl, morpholinyl, thianyl, thianyl sulfoxide, 1-oxo-1-imino-lthiacyclohexyl, dithianyl, thiomorpholinyl, oxetanyl, tetrahydrofuranyl, tetrahydropyranyl, or tetrahydrothiophenyl.

In some embodiments, R¹ and R^{1b} are an unsubstituted 4-, 5-, or 6-membered heterocycloalkyl, such as, for example, unsubstituted: azetidinyl, oxetanyl, pyrrolidinyl, dioxolanyl, imidazolidinyl, pyrazolidinyl, piperazinyl, piperidinyl, dioxanyl, morpholinyl, thianyl, dithianyl, thiomorpholinyl, oxetanyl, tetrahydrofuranyl, tetrahydropyranyl, or tetrahydrothiophenyl.

In some embodiments, R¹ and R^{1b} are an optionally substituted 4-membered heterocycloalkyl, such as, for example, optionally substituted: azetidinyl or oxetanyl.

In some embodiments, R¹ and R^{1b} are an optionally substituted 5-membered heterocycloalkyl, such as, for example, optionally substituted: pyrrolidinyl, dioxolanyl, imidazolidinyl, pyrazolidinyl, tetrahydrofuranyl, or tetrahydrothiophenyl.

In some embodiments, R¹ and R^{1b} are an optionally substituted 6-membered heterocycloalkyl, such as, for example, optionally substituted: piperazinyl, piperidinyl, dioxanyl, morpholinyl, thianyl, dithianyl, thiomorpholinyl, or tetrahydropyranyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are optionally substituted imidazolidinonyl, optionally substituted pyrrolidinonyl, optionally substituted dihydropyrazolonyl, optionally substituted dihydropyrrolonyl, optionally substituted dihydroimidazolonyl, or optionally substituted dihydrotriazolonyl.

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are optionally substituted pyrrolidinyl.

In some embodiments, R¹ and R^{1b} are unsubstituted pyrrolidinyl, such as, for example,

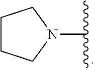 [SMILES: *N1CCCC1]

In some embodiments, R¹ and R^{1b} are pyrrolidinyl substituted with one or more of oxo, C₁-C₆hydroxyalkyl, C₁-C₆alkyl, C₁-C₆alkoxy(alkylene), C₁-C₆cyanoalkyl, OH, cyano, C(O)NR^{b}R^{c}, wherein each R^{b} and R^{c} is independently hydrogen or C₁-C₄alkyl. In other embodiments, R¹ and R^{1b} are pyrrolidinyl substituted with one or more of oxo, C(CH₃)₂OH, methyl, CH₂OH, CH₂OCH₃, OH, C(O)NH₂, cyano, or CH₂CN. In yet other embodiments, R¹ and R^{1b} are

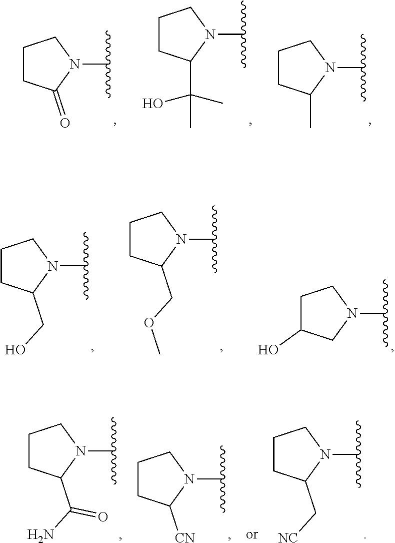

In some embodiments, R¹ in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, or Formula I-K-1 and R^{1b} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 are optionally substituted tetrahydrofuranyl. In other embodiments, R¹ and R^{1b} are an unsubstituted tetrahydrofuranyl, such as, for example,

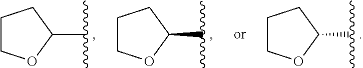

In some embodiments, R^{1a} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is hydrogen.

In some embodiments, R^{1a} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is fluoro.

In some embodiments, R^{1c} in the compounds of Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is hydrogen.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is a bond.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is —O—.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is —NR^{a}—, wherein R^{a} is hydrogen or C₁-C₄alkyl.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₁-C₄alkylene.

In some embodiments, L is C₁-C₄alkylene substituted with deuterium, oxo, halo, NH₂, NH(C₁-C₄alkyl), N(C₁-C₄alkyl)₂, OH, C₁-C₃alkoxy, NR¹C(O)(C₁-C₄alkyl), NR¹CO₂(C₁-C₄alkyl), or an optionally substituted 4-, 5-, or 6-membered heterocycloalkyl containing a nitrogen wherein the nitrogen is connected to the alkylene carbon and each R¹ is independently hydrogen or C₁-C₄alkyl.

In some embodiments, L is unsubstituted C₁-C₄alkylene.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted phenyl, optionally substituted pyridinyl, optionally substituted C₁-C₆alkyl, optionally substituted C₁-C₆alkoxy, or NR^{b}R^{c}, wherein R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl.

In some embodiments, R² is phenyl or pyridinyl, each optionally substituted with one or more of halo, C₁-C₆alkyl, C₃-C₆cycloalkyl, C₁-C₆alkoxy, C₁-C₆haloalkoxy, cyano, OH, NR^{m}Rⁿ, C(O)NR^{f}R^{g}, CO₂(C₁-C₆alkyl), COOH, C₁-C₄alkylene-R²⁷, or O—R²⁸, wherein:
R²⁷ is cyano, NR^{m}Rⁿ, or COOH;
R²⁸ is C₁-C₆hydroxyalkyl, C₃-C₆cycloalkyl, optionally substituted 4-, 5-, or 6-membered heterocycloalkyl, or (C₁-C₄alkylene)C₁-C₄alkoxy; and
each R^{f}, R^{g}, R^{m}, and Rⁿ is independently hydrogen or C₁-C₄alkyl.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is a bond; and R² is

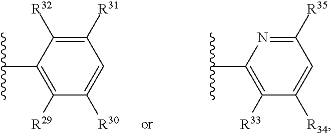

wherein:
R²⁹, R³⁰, R³¹, R³², R³³, R³⁴, and R³⁵ are each independently hydrogen, halo, OH, cyano, C₁-C₄alkoxy, C₁-C₄haloalkoxy, C(O)NR^{f}R^{g}, or O—R²⁸.
R²⁸ is C₁-C₆hydroxyalkyl or (C₁-C₄alkylene)C₁-C₄alkoxy; and
wherein each Rand R^{g} is independently hydrogen or C₁-C₄alkyl.

In some embodiments, L in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is a bond, —O—, —NR^{a}—, or optionally substituted C₁-C₄alkylene, wherein R^{a} is hydrogen or C₁-C₄alkyl.

In some embodiments, L is —NR^{a}—, wherein R^{a} is hydrogen or C₁-C₄alkyl. In embodiments in which L is —NR^{a}— and R^{a} is hydrogen, L is —NH—. In embodiments in which L is —NR^{a}— and R^{a} is C₁-C₆alkyl, L is —N(C₁-C₆alkyl)-, such as, for example, —N(C₆alkyl)-, —N(C₅alkyl)-, —N(C₄alkyl)-, —N(C₃alkyl)-, —N(C₂alkyl)- (e.g., —N(CH₂CH₃)—), —N(C₁alkyl)- (e.g., —N(CH₃)—).

In some embodiments, L is optionally substituted C₁-C₄alkylene, such as, for example, optionally substituted: C₄alkylene, C₃alkylene, C₂alkylene (e.g., —CH₂CH₂—), C₁alkylene (e.g., —CH₂—), and the like.

In some embodiments, L is substituted C₁-C₄alkylene, wherein the C₁-C₄alkylene is substituted with deuterium, oxo, halo, NH₂, NH(C₁-C₄alkyl), N(C₁-C₄alkyl)₂, OH, C₁-C₃alkoxy, NR¹C(O)(C₁-C₄alkyl), NR¹CO₂(C₁-C₄alkyl), or an optionally substituted 4-, 5-, or 6-membered heterocycloalkyl containing a nitrogen wherein the nitrogen is connected to the alkylene carbon and each R¹ is independently hydrogen or C₁-C₄alkyl.

In some embodiments, L is substituted C₁-C₄alkylene, wherein the C₁-C₄alkylene is substituted with OH, NH₂, oxo, F, OCH₃, NHC(O)CH₃, or NHCO₂CH₃.

In some embodiments, L is —CHOH—, —CHNH₂—, —C(O)—, —CHF—, —CF₂—, —CH(OCH₃)—, —CH(NHC(O)CH₃)—, or —CHNH(CO₂CH₃)—.

In some embodiments, L is substituted C₁-C₄alkylene, wherein the C₁-C₄alkylene is substituted with deuterium.

In some embodiments, L is—CHD- or —CD₂-.

In some embodiments, L is substituted C₁-C₄alkylene, wherein the C₁-C₄alkylene is substituted with NHC(O)CH₃, NHC(O)OCH₃,

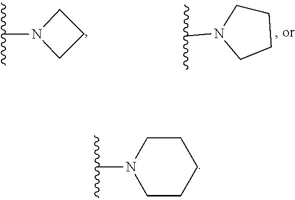 [SMILES: *N1CCC1 ; *N1CCCC1 ; *N1CCCCC1]

In some embodiments, L is unsubstituted C₁-C₄alkylene, such as, for example, unsubstituted: C₄alkylene, C₃alkylene, C₂alkylene (e.g., —CH₂CH₂—), C₁alkylene (e.g., —CH₂—), and the like.

In some embodiments, L is —CH₂—.

In some embodiments, L is —CH₂CH₂—.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is

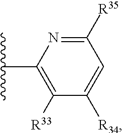

wherein R³⁴ and R³⁵ are each hydrogen, and R³³ is hydrogen, halo, or C₁-C₄haloalkoxy. In other embodiments, R³³ is hydrogen, F, or OCHF₂.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is

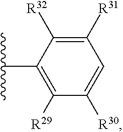

wherein at least two of R²⁹, R³⁰, R³¹, and R³² are hydrogen and R²⁹, R³⁰, R³¹, and R³² are defined herein. In other embodiments, one of R²⁹ and R³² is halo, OH, C₁-C₄alkoxy, or C₁-C₄haloalkoxy and the other of R²⁹ and R³² is halo; and R³⁰ and R³¹ are hydrogen. In yet other embodiments, R²⁹ is halo, cyano, OH, C₁-C₄haloalkoxy, C₁-C₄alkoxy, C(O)NR^{f}R^{g}, or O—R²⁸, and R³⁰, R³¹, and R³² are each hydrogen.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is

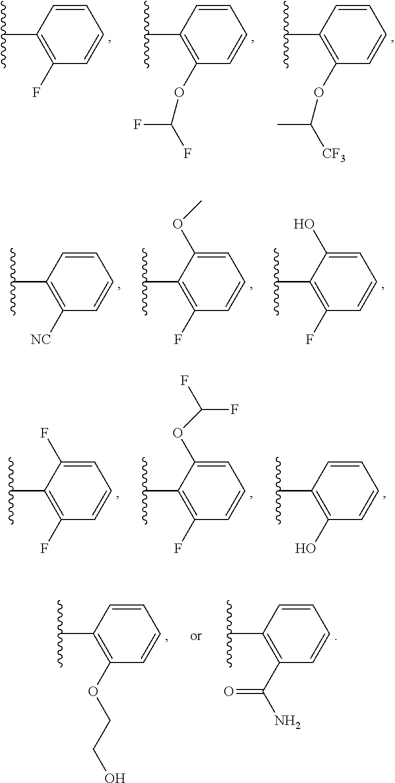

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is C₁-C₆alkyl, optionally substituted with cyano, OH, or C(O)NRⁱR^{j}, and wherein each R and RJ is independently hydrogen or C₁-C₄alkyl. In other embodiments, R² is CH₃, CH₂CH₃, CH(CH₃)₂, CH₂CH(CH₃)₂, C(CH₃)₃, CH₂C(CH₃)₃, C(CH₃)₂OH, CH(OH)(CH₃), C(CH₃)₂CN, C(CH₃)₂C(O)NH₂, or C(CH₃)(CH₂CH₂OH).

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is C₁-C₆alkoxy, optionally substituted with OH. In other embodiments, R² is OCH₃, OCH(CH₃)₂, or OCH₂CH₂OH.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is C₁-C₆haloalkyl or C₁-C₆haloalkoxy. In other embodiments, R² is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, CF₂CH(CH₃)₂, OCF₃, OCH₂CF₃, OCHF₂, OCH₂F, or OCH(CH₃)(CF₃).

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is C₃-C₆cycloalkyl, optionally substituted with one or more of C₁-C₄alkyl or C₁-C₄haloalkyl.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is NR^{b}R^{c}, wherein R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl. In other embodiments, R² is NR^{b}R^{c}, wherein R^{b} and R^{c} are each independently hydrogen or C₁-C₄alkyl. In yet other embodiments, R² is NH(CH₃), N(CH₃)₂, N(CH₃)(CH₂CH₃), or NH(CH₂CH₃).

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 5-membered heteroaryl, such as optionally substituted triazolyl, optionally substituted pyrazolyl, optionally substituted oxazolyl, or optionally substituted isoxazolyl.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 4-, 5- or 6-membered heterocycloalkyl, such as optionally substituted azetidinyl, optionally substituted tetrahydrofuranyl, optionally substituted pyrrolidinyl, optionally substituted tetrahydropyranyl, optionally substituted piperidinyl, optionally substituted morpholinyl, or optionally substituted dioxanyl.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted phenyl, optionally substituted pyridinyl, optionally substituted 5-membered heteroaryl, optionally substituted 4-, 5-, or 6-membered heterocycloalkyl, optionally substituted C₁-C₆alkyl, optionally substituted C₁-C₆alkoxy, optionally substituted C₁-C₆hydroxyalkyl, optionally substituted C₁-C₆haloalkyl, optionally substituted C₁-C₆haloalkoxy, optionally substituted C₃-C₆cycloalkyl, or NR^{b}R^{c}, wherein R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted phenyl or optionally substituted pyridinyl.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted phenyl.

In some embodiments, R² is an unsubstituted phenyl.

In some embodiments, R² is a substituted phenyl. In some embodiments, the phenyl may be substituted with 1, 2, or 3, independently selected substituents.

In some embodiments, R² is a phenyl substituted with one or more of halo, C₁-C₆alkyl, C₃-C₆cycloalkyl, C₁-C₆alkoxy, C₁-C₆haloalkoxy, cyano, OH, NR^{m}Rⁿ, C(O)NR^{f}R^{g}, C₀₂(C₁-C₆alkyl), COOH, C₁-C₄alkylene-R²⁷, or O—R²⁸ wherein R²⁷ is cyano, NR^{m}Rⁿ, or COOH; R²⁸ is C₁-C₆hydroxyalkyl, C₃-C₆cycloalkyl, optionally substituted 4-, 5-, or 6-membered heterocycloalkyl, or (C₁-C₄alkylene)C₁-C₄alkoxy; and each R^{f}, R^{g}, R^{m}, and Rⁿ is independently hydrogen or C₁-C₄alkyl.

In some embodiments, R² is

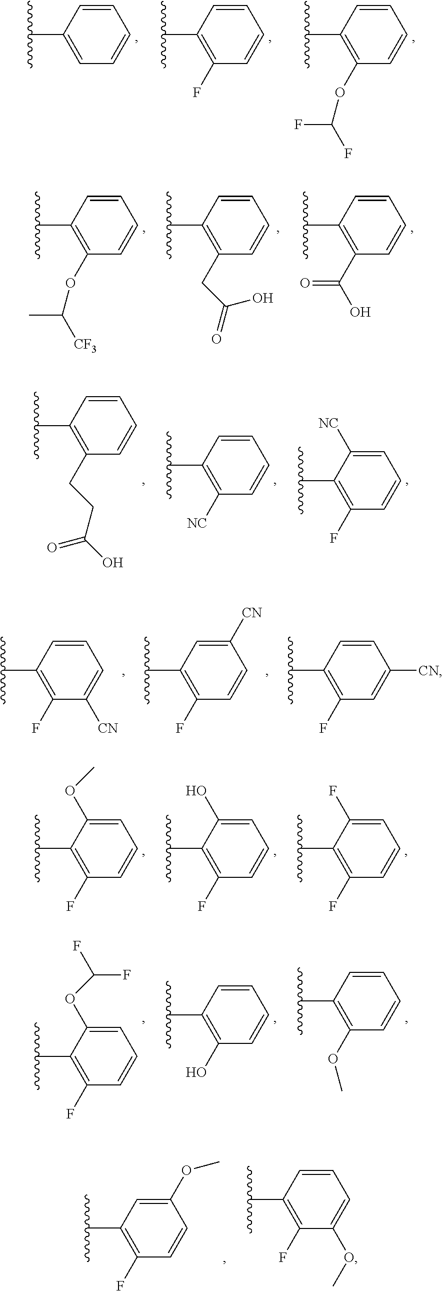

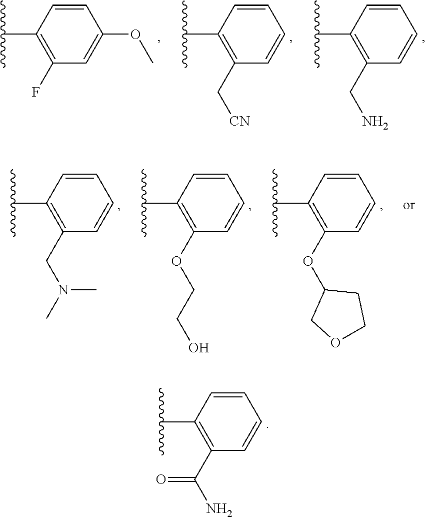

In some embodiments, R² is phenyl substituted with 1, 2, 3, 4, or 5 deuterium atoms. In some embodiments, the phenyl with 1, 2, 3, or 4 deuterium atoms may be additionally substituted with one or more of halo, C₁-C₆alkyl, cyano, or C₁-C₆haloalkyl. In some embodiments, R² is

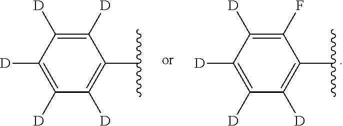 [SMILES: *c1c([2H])c([2H])c([2H])c([2H])c1[2H] ; *c1c([2H])c([2H])c([2H])c([2H])c1F]

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted pyridinyl. In some embodiments, the optionally substituted pyridinyl may attach to L at any carbon atom in the pyridinyl ring, i.e.,

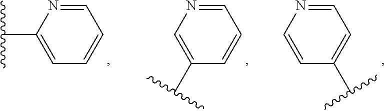

and the like, wherein

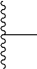

indicates the point of attachment to L.

In some embodiments, R² is an unsubstituted pyridinyl.

In some embodiments, R² is

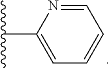 [SMILES: *c1ccccn1]

In some embodiments, R² is a substituted pyridinyl. In some embodiments, the pyridinyl may be substituted with 1, 2, or 3, independently selected substituents.

In some embodiments, R² is a pyridinyl substituted with one or more of halo, C₁-C₆alkyl, C₃-C₆cycloalkyl, C₁-C₆alkoxy, C₁-C₆haloalkoxy, cyano, OH, NR^{m}Rⁿ, C(O)NR^{f}R^{g}, CO₂(C₁-C₆alkyl), COOH, C₁-C₄alkylene-R²⁷, or O—R²⁸ wherein R²⁷ is cyano, NR^{m}Rⁿ, or COOH; R²⁸ is C₁-C₆hydroxyalkyl, C₃-C₆cycloalkyl, optionally substituted 4-, 5-, or 6-membered heterocycloalkyl, or (C₁-C₄alkylene)C₁-C₄alkoxy; and each R^{f}, R^{g}, R^{m}, and Rⁿ is independently hydrogen or C₁-C₄alkyl.

In some embodiments, R² is a pyridinyl, where the pyridinyl nitrogen atom is substituted by an oxide (═O) to form an N-oxide, such as, for example,

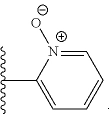 [SMILES: *c1cccc[n+]1[O-]]

In some embodiments, R² is

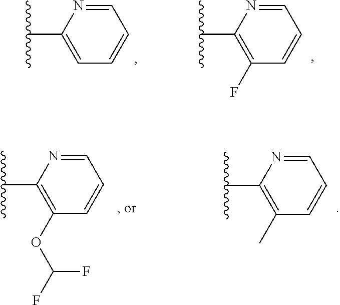

In some embodiments, R² is pyridinyl substituted with 1, 2, 3, or 4 deuterium atoms. In further embodiments, the pyridinyl with 1, 2, or 3 deuterium atoms may be additionally substituted with one or more of halo, C₁-C₆alkyl, or C₁-C₆haloalkyl. In yet some embodiments, R² is

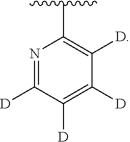

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 5-membered heteroaryl, such as, for example, optionally substituted: pyrrolyl, pyrazolyl, imidazolyl, triazolyl, tetrazolyl, oxadiazolyl, oxazolyl, isoxazolyl, isothiazolyl, thiazolyl, or thiadiazolyl. In some embodiments, the optionally substituted 5-membered heteroaryl may attach to L at a carbon atom or a nitrogen atom in the 5-membered heteroaryl ring.

In some embodiments, R² is optionally substituted triazolyl, optionally substituted pyrazolyl, optionally substituted oxazolyl, or optionally substituted isoxazolyl.

In some embodiments, R² is an unsubstituted 5-membered heteroaryl.

In some embodiments, R² is an unsubstituted pyrazolyl. In some embodiments, R² is

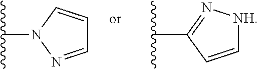 [SMILES: *n1cccn1 ; *c1cc[nH]n1]

In some embodiments, R² is an unsubstituted triazolyl. In some embodiments, R² is

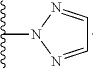 [SMILES: *n1nccn1]

In some embodiments, R² is a substituted 5-membered heteroaryl. In some embodiments, the 5-membered heteroaryl may be substituted with 1, 2, or 3, independently selected substituents. In some embodiments, R² is a 5-membered heteroaryl substituted with one or more of halo, C₁-C₆alkyl, or C₁-C₆haloalkyl. In other embodiments, R² is a 5-membered heteroaryl substituted with one or more C₁-C₆alkyl. In yet other embodiments, R² is a 5-membered heteroaryl substituted with one or more CH₃. In some embodiments, R² is

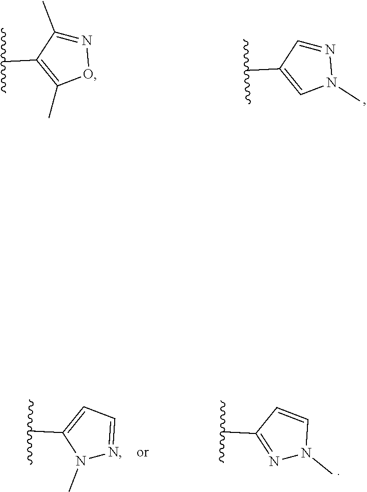

In some embodiments, R²

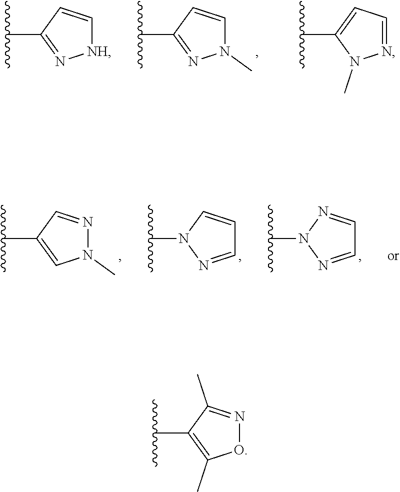

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted 4-, 5-, or 6-membered heterocycloalkyl, such as, for example, optionally substituted: azetidinyl, tetrahydrofuranyl, pyrrolidinyl, piperidinyl, morpholinyl, dioxanyl, tetrahydropyranyl, thianyl, thianyl sulfone, thianyl sulfoxide, or 1-oxo-1-imino-1-thiacyclohexyl. In some embodiments, the optionally substituted heterocycloalkyl may attach to L at a carbon atom or a nitrogen atom in the heterocycloalkyl ring.

In some embodiments, R² is optionally substituted azetidinyl, optionally substituted tetrahydrofuranyl, optionally substituted pyrrolidinyl, optionally substituted tetrahydropyranyl, optionally substituted piperidinyl, optionally substituted morpholinyl, or optionally substituted dioxanyl.

In some embodiments, R² is optionally substituted tetrahydrofuranyl, optionally substituted optionally substituted piperidinyl, optionally substituted morpholinyl, optionally substituted dioxanyl, or optionally substituted tetrahydropyranyl.

In some embodiments, R² is an unsubstituted 4-, 5-, or 6-membered heterocycloalkyl.

In some embodiments, R² is

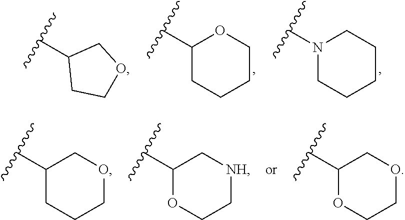

In other embodiments, R² is

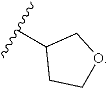 [SMILES: *C1CCOC1]

In some embodiments, R² is a substituted 4-, 5-, or 6-membered heterocycloalkyl. In some embodiments, the heterocycloalkyl may be substituted with 1, 2, or 3, independently selected substituents.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₁-C₆alkyl, such as, for example, optionally substituted: C₆alkyl, C₅alkyl, C₄alkyl such as CH₂CH(CH₃)₂, C₃alkyl such as CH(CH₃)₂, C₂alkyl, or C₁alkyl (e.g., CH₃).

In some embodiments, R² is unsubstituted C₁-C₆alkyl.

In some embodiments, R² is CH₃, CH₂CH₃, CH(CH₃)₂, CH₂CH(CH₃)₂, C(CH₃)₃, or CH₂C(CH₃)₃.

In some embodiments, R² is CH₃.

In some embodiments, R² is CH₂CH₃.

In some embodiments, R² is CH₂CH(CH₃)₂.

In some embodiments, R² is CH(CH₃)₂.

In some embodiments, R² is C(CH₃)₃.

In some embodiments, R² is CH₂C(CH₃)₃.

In some embodiments, R² is C₁-C₆alkyl, substituted with cyano, OH, or C(O)NRⁱR^{j}, wherein each Rⁱ and R^{j} is independently hydrogen or C₁-C₄alkyl.

In some embodiments, R² is CH₃, CH₂CH₃, CH(CH₃)₂, CH₂CH(CH₃)₂, C(CH₃)₃, CH₂C(CH₃)₃, C(CH₃)₂OH, CH(OH)(CH₃), C(CH₃)₂CN, C(CH₃)₂C(O)NH₂, or C(CH₃)(CH₂CH₂OH).

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₁-C₆alkoxy, such as, for example, optionally substituted: C₆alkoxy, C₅alkoxy, C₄alkoxy, C₃alkoxy such as OCH(CH₃)₂, C₂alkoxy, or C₁alkoxy (e.g., OCH₃).

In some embodiments, R² is unsubstituted C₁-C₆alkoxy.

In some embodiments, R² is OCH₃ or OCH(CH₃)₂.

In some embodiments, R² is C₁-C₆alkoxy, optionally substituted with OH.

In some embodiments, R² is OCH₃, OCH(CH₃)₂, or OCH₂CH₂OH.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₁-C₆haloalkyl, such as, for example, optionally substituted: C₆haloalkyl, C₅haloalkyl, C₄haloalkyl, C₃haloalkyl such as CH(CH₃)(CF₃), C₂haloalkyl such as CH₂CF₃, or C₁haloalkyl such as CF₃, CHF₂ or CH₂F.

In some embodiments, R² is unsubstituted C₁-C₆haloalkyl.

In some embodiments, R² is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, or CF₂CH(CH₃)₂.

In some embodiments, R² is CF₃.

In some embodiments, R² is CH₂CF₃.

In some embodiments, R² is CH(CH₃)(CF₃).

In some embodiments, R² is C(CH₃)₂F.

In some embodiments, R² is CH₂C(CH₃)₂F.

In some embodiments, R² is C(CH₃)F₂.

In some embodiments, R² is CH₂C(CH₃)F₂.

In some embodiments, R² is CHFCH(CH₃)₂.

In some embodiments, R² is CF₂CH(CH₃)₂.

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₁-C₆haloalkoxy, such as, for example, optionally substituted: C₆haloalkoxy, C₅haloalkoxy, C₄haloalkoxy, C₃haloalkoxy such as OCH(CH₃)(CF₃), C₂haloalkoxy such as OCH₂CF₃, or C₁haloalkoxy such as OCF₃, OCHF₂, or OCH₂F.

In some embodiments, R² is unsubstituted C₁-C₆haloalkoxy.

In some embodiments, R² is OCF₃, OCH₂CF₃, OCHF₂, OCH₂F, or OCH(CH₃)(CF₃).

In some embodiments, R² is OCF₃.

In some embodiments, R² is OCH₂CF₃.

In some embodiments, R² is OCHF₂.

In some embodiments, R² is OCH₂F.

In some embodiments, R² is OCH(CH₃)(CF₃).

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is optionally substituted C₃-C₆cycloalkyl.

In some embodiments, R² is unsubstituted C₃-C₆cycloalkyl.

In some embodiments, R² is C₃-C₆cycloalkyl, optionally substituted with one or more of C₁-C₄alkyl or C₁-C₄haloalkyl.

In some embodiments, R² is cyclopropyl, cyclobutyl,

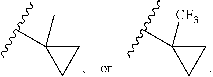

In some embodiments, R² in the compounds of Formula I, Formula I-A, Formula I-B, Formula I-C, Formula I-D, Formula I-E, Formula I-F, Formula I-G, Formula I-G-1, Formula I-H, Formula I-H-1, Formula I-J, Formula I-J-1, Formula I-K, Formula I-K-1, Formula II, Formula II-A, Formula II-B, Formula II-C, Formula II-D, Formula II-E, Formula II-F, Formula II-G, Formula II-G-1, Formula II-H, Formula II-H-1, Formula II-J, Formula II-J-1, Formula II-K, or Formula II-K-1 is NR^{b}R^{c}, wherein R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl.

In some embodiments, R² is NH₂, NH(CH₃), N(CH₃)₂, NH(CH(CH₃)₂), N(CH₃)(CH₂CH₃), N(CH₃)(cyclopropyl), NH(CH₂CH₃), or N(CH₃)(CH₂CH₃).

In some embodiments, the compound is a compound of Formula I selected from Table 1 or Table 2, or a pharmaceutically acceptable salt thereof. In other embodiments, the compound is a compound of Formula I selected from Table 1, or a pharmaceutically acceptable salt thereof. In yet other embodiments, the compound is a compound of Formula I selected from Table 2, or a pharmaceutically acceptable salt thereof.

In some embodiments, the compound is a compound of Formula II selected from Table 3 or Table 4, or a pharmaceutically acceptable salt thereof. In other embodiments, the compound is a compound of Formula II selected from Table 3, or a pharmaceutically acceptable salt thereof. In yet other embodiments, the compound is a compound of Formula II selected from Table 4, or a pharmaceutically acceptable salt thereof.

In some embodiments, compounds of the disclosure are any one or more of the compounds of Tables 1, 2, 3, and 4, and their pharmaceutically acceptable salts and/or isotopologues. Compounds having the Formulas I and II are further disclosed in the Exemplification and are included in the present disclosure. Pharmaceutically acceptable salts thereof as well as the neutral forms are included.

In another embodiment, the compounds of the disclosure may be enantiomerically enriched, e.g., the enantiomeric excess or “ee” of the compound is greater than about 5% as measured by chiral HPLC. In some embodiments, the ee is greater than about 10%. In some embodiments, the ee is greater than about 20%. In further embodiments, the ee is greater than about 30%. In yet some embodiments, the ee is greater than about 40%. In still further embodiments, the ee is greater than about 50%. In some embodiments, the ee is greater than about 60%. In further embodiments, the ee is greater than about 70%. In still some embodiments, the ee is greater than about 80%. In yet further embodiments, the ee is greater than about 85%. In some embodiments, the ee is greater than about 90%. In further embodiments, the ee is greater than about 91%. In yet some embodiments, the ee is greater than about 92%. In still further embodiments, the ee is greater than about 93%. In some embodiments, the ee is greater than about 94%. In further embodiments, the ee is greater than about 95%. In still some embodiments, the ee is greater than about 96%. In yet further embodiments, the ee is greater than about 97%. In some embodiments, the ee is greater than about 98%. In further embodiments, the ee is greater than about 99%.

The present disclosure encompasses the preparation and use of salts of compounds of the disclosure. Salts of compounds of the disclosure can be prepared during the final isolation and purification of the compounds or separately by reacting the compound with an acid or base as appropriate.

### EXAMPLES

In some embodiments, the disclosure provides specific examples of Formula I, and their pharmaceutically acceptable salts and/or isotopologues, as set forth in Table 1 below.

TABLE 1

| Ex. | | |
|---|---|---|
| No. | Structure | Name |
| 1 | 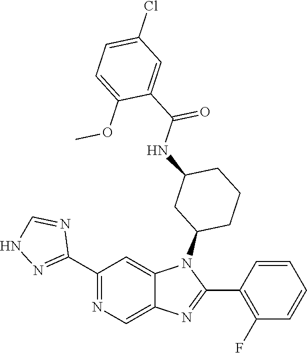 [SMILES: COc1ccc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-methoxybenzamide |
| 2 | 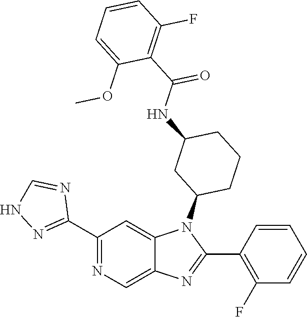 [SMILES: COc1cccc(F)c1C(=O)N[C@H]1CCC[C@@H](n2c(-c3ccccc3F)nc3cnc(-c4nc[nH]n4)cc32)C1] | 2-fluoro-N-((1S,3R)-3-(2-(2-fluorophenyl)-6-(1H-1,2,4- triazol-3-yl)-1H-imidazo[4,5-c]pyridin-1-yl)cyclohexyl)- 6-methoxybenzamide |
| 3 | 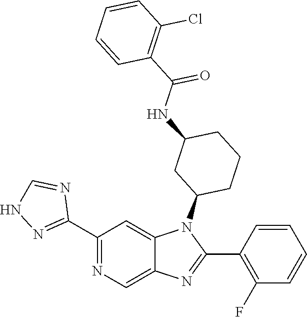 [SMILES: O=C(N[C@H]1CCC[C@@H](n2c(-c3ccccc3F)nc3cnc(-c4nc[nH]n4)cc32)C1)c1ccccc1Cl] | 2-chloro-N-((1S,3R)-3-(2-(2-fluorophenyl)-6-(1H-1,2,4- triazol-3-yl)-1H-imidazo[4,5-c]pyridin-1- yl)cyclohexyl)benzamide |
| 4 | 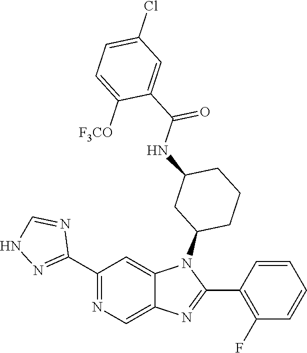 [SMILES: O=C(N[C@H]1CCC[C@@H](n2c(-c3ccccc3F)nc3cnc(-c4nc[nH]n4)cc32)C1)c1cc(Cl)ccc1OC(F)(F)F] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(trifluoromethoxy)benzamide |
| 5 | 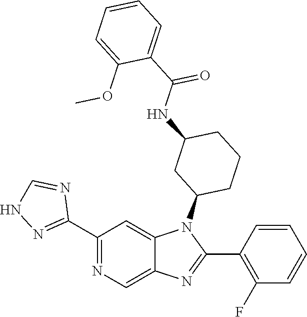 [SMILES: COc1ccccc1C(=O)N[C@H]1CCC[C@@H](n2c(-c3ccccc3F)nc3cnc(-c4nc[nH]n4)cc32)C1] | N-((1S,3R)-3-(2-(2-fluorophenyl)-6-(1H-1,2,4-triazol-3- yl)-1H-imidazo[4,5-c]pyridin-1-yl)cyclohexyl)-2- methoxybenzamide |
| 6 | 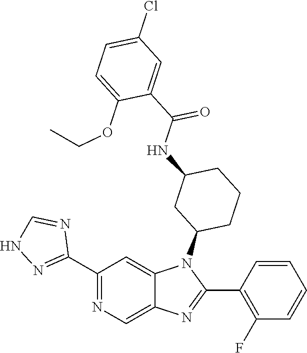 [SMILES: CCOc1ccc(Cl)cc1C(=O)N[C@H]1CCC[C@@H](n2c(-c3ccccc3F)nc3cnc(-c4nc[nH]n4)cc32)C1] | 5-chloro-2-ethoxy-N-((1S,3R)-3-(2-(2-fluorophenyl)-6- (1H-1,2,4-triazol-3-yl)-1H-imidazo[4,5-c]pyridin-1- yl)cyclohexyl)benzamide |
| 7 | 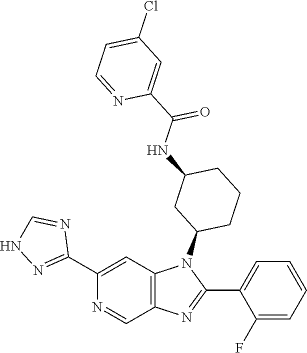 [SMILES: O=C(N[C@H]1CCC[C@@H](n2c(-c3ccccc3F)nc3cnc(-c4nc[nH]n4)cc32)C1)c1cc(Cl)ccn1] | 4-chloro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 8 | 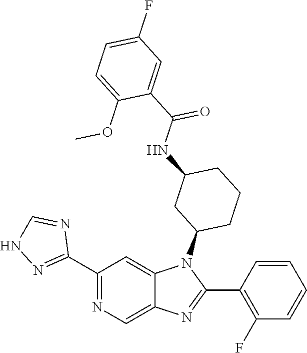 [SMILES: COc1ccc(F)cc1C(=O)N[C@H]1CCC[C@@H](n2c(-c3ccccc3F)nc3cnc(-c4nc[nH]n4)cc32)C1] | 5-fluoro-N-((1S,3R)-3-(2-(2-fluorophenyl)-6-(1H-1,2,4- triazol-3-yl)-1H-imidazo[4,5-c]pyridin-1-yl)cyclohexyl)- 2-methoxybenzamide |
| 9 | 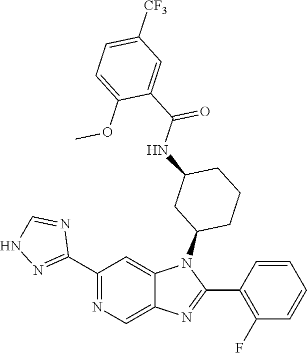 [SMILES: COc1ccc(C(F)(F)F)cc1C(=O)N[C@H]1CCC[C@@H](n2c(-c3ccccc3F)nc3cnc(-c4nc[nH]n4)cc32)C1] | N-((1S,3R)-3-(2-(2-fluorophenyl)-6-(1H-1,2,4-triazol-3- yl)-1H-imidazo[4,5-c]pyridin-1-yl)cyclohexyl)-2- methoxy-5-(trifluoromethyl)benzamide |
| 10 | 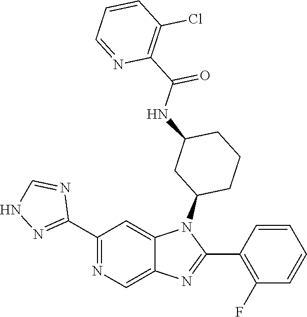 [SMILES: O=C(N[C@H]1CCC[C@@H](n2c(-c3ccccc3F)nc3cnc(-c4nc[nH]n4)cc32)C1)c1ncccc1Cl] | 3-chloro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 11 | 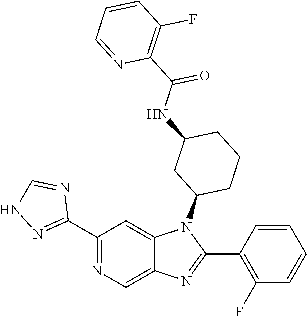 [SMILES: O=C(N[C@H]1CCC[C@@H](n2c(-c3ccccc3F)nc3cnc(-c4nc[nH]n4)cc32)C1)c1ncccc1F] | 3-fluoro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 12 | 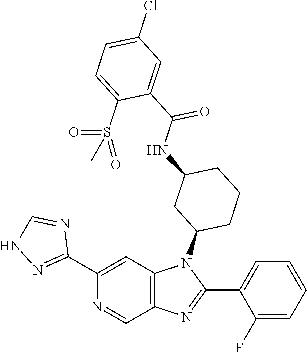 [SMILES: CS(=O)(=O)c1ccc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(methylsulfonyl)benzamide |
| 13 | 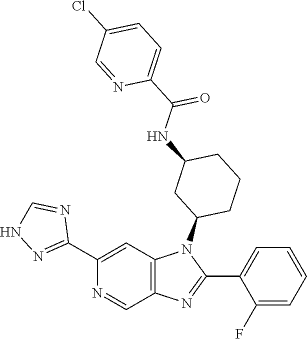 [SMILES: O=C(N[C@H]1CCC[C@@H](n2c(-c3ccccc3F)nc3cnc(-c4nc[nH]n4)cc32)C1)c1ccc(Cl)cn1] | 5-chloro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 14 | 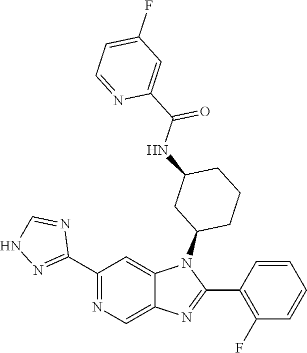 [SMILES: O=C(N[C@H]1CCC[C@@H](n2c(-c3ccccc3F)nc3cnc(-c4nc[nH]n4)cc32)C1)c1cc(F)ccn1] | 4-fluoro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 15 | 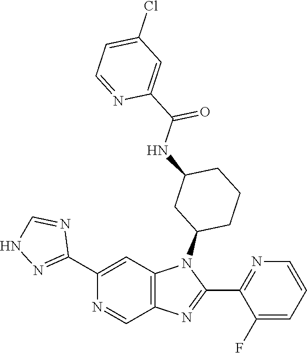 [SMILES: O=C(N[C@H]1CCC[C@@H](n2c(-c3ncccc3F)nc3cnc(-c4nc[nH]n4)cc32)C1)c1cc(Cl)ccn1] | 4-chloro-N-((1S,3R)-3-(2-(3-fluoropyridin-2-yl)-6-(1H- 1,2,4-triazol-3-yl)-1H-imidazo[4,5-c]pyridin-1- yl)cyclohexyl)picolinamide |
| 16 | 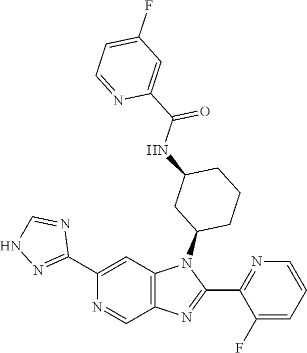 [SMILES: O=C(N[C@H]1CCC[C@@H](n2c(-c3ncccc3F)nc3cnc(-c4nc[nH]n4)cc32)C1)c1cc(F)ccn1] | 4-fluoro-N-((1S,3R)-3-(2-(3-fluoropyridin-2-yl)-6-(1H- 1,2,4-triazol-3-yl)-1H-imidazo[4,5-c]pyridin-1- yl)cyclohexyl)picolinamide |
| 17 | 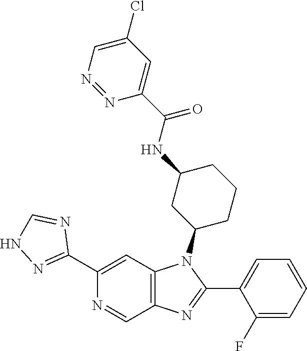 [SMILES: O=C(N[C@H]1CCC[C@@H](n2c(-c3ccccc3F)nc3cnc(-c4nc[nH]n4)cc32)C1)c1cc(Cl)cnn1] | 5-chloro-N-((1S,3R)-3-(2-(2-fluorophenyl)-6-(1H-1,2,4- triazol-3-yl)-1H-imidazo[4,5-c]pyridin-1- yl)cyclohexyl)pyridazine-3-carboxamide |
| 18 | 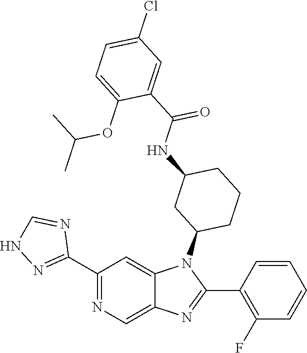 [SMILES: CC(C)Oc1ccc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-isopropoxybenzamide |
| 19 | 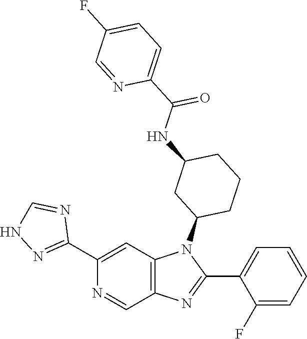 [SMILES: O=C(N[C@H]1CCC[C@@H](n2c(-c3ccccc3F)nc3cnc(-c4nc[nH]n4)cc32)C1)c1ccc(F)cn1] | 5-fluoro-N-((1S,3R)-3-(2-(2-fluorophenyl)-6-(1H-1,2,4- triazol-3-yl)-1H-imidazo[4,5-c]pyridin-1- yl)cyclohexyl)picolinamide |
| 20 | 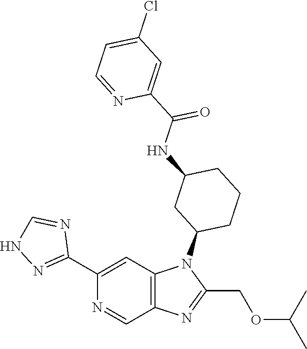 [SMILES: CC(C)OCc1nc2cnc(-c3nc[nH]n3)cc2n1[C@@H]1CCC[C@H](NC(=O)c2cc(Cl)ccn2)C1] | 4-chloro-N-((1S,3R)-3-(2-(isopropoxymethyl)-6-(1H- 1,2,4-triazol-3-yl)-1H-imidazo[4,5-c]pyridin-1- yl)cyclohexyl)picolinamide |
| 21 | 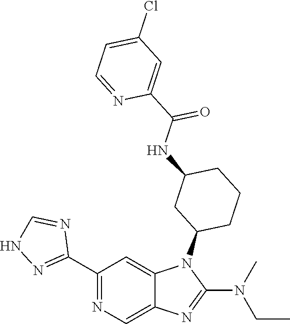 [SMILES: CCN(C)c1nc2cnc(-c3nc[nH]n3)cc2n1[C@@H]1CCC[C@H](NC(=O)c2cc(Cl)ccn2)C1] | 4-chloro-N-((1S,3R)-3-(2-(ethyl(methyl)amino)-6-(1H- 1,2,4-triazol-3-yl)-1H-imidazo[4,5-c]pyridin-1- yl)cyclohexyl)picolinamide |
| 22 | 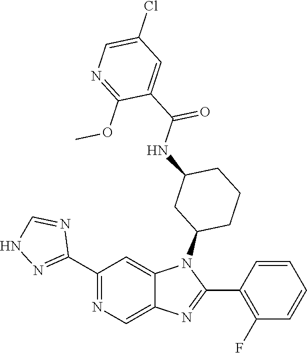 [SMILES: COc1ncc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-methoxynicotinamide |
| 23 | 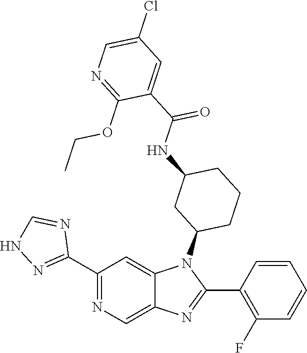 [SMILES: CCOc1ncc(Cl)cc1C(=O)N[C@H]1CCC[C@@H](n2c(-c3ccccc3F)nc3cnc(-c4nc[nH]n4)cc32)C1] | 5-chloro-2-ethoxy-N-((1S,3R)-3-(2-(2-fluorophenyl)-6- (1H-1,2,4-triazol-3-yl)-1H-imidazo[4,5-c]pyridin-1- yl)cyclohexyl)nicotinamide |
| 24 | 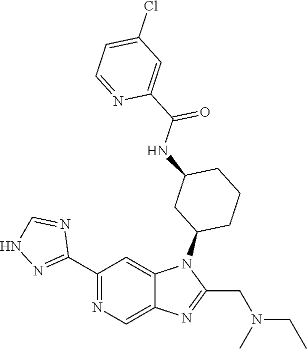 [SMILES: CCN(C)Cc1nc2cnc(-c3nc[nH]n3)cc2n1[C@@H]1CCC[C@H](NC(=O)c2cc(Cl)ccn2)C1] | 4-chloro-N-((1S,3R)-3-(2-((ethyl(methyl)amino)methyl)- 6-(1H-1,2,4-triazol-3-yl)-1H-imidazo[4,5-c]pyridin-1- yl)cyclohexyl)picolinamide |
| 25 | 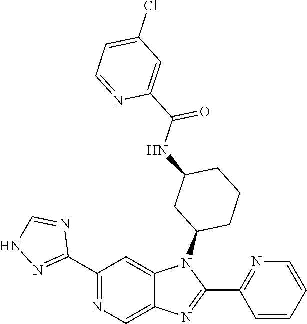 [SMILES: O=C(N[C@H]1CCC[C@@H](n2c(-c3ccccn3)nc3cnc(-c4nc[nH]n4)cc32)C1)c1cc(Cl)ccn1] | 4-chloro-N-((1S,3R)-3-(2-(pyridin-2-yl)-6-(1H-1,2,4- triazol-3-yl)-1H-imidazo[4,5-c]pyridin-1- yl)cyclohexyl)picolinamide |
| 26 | 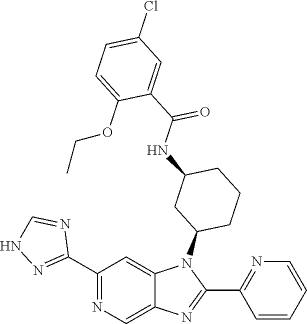 [SMILES: CCOc1ccc(Cl)cc1C(=O)N[C@H]1CCC[C@@H](n2c(-c3ccccn3)nc3cnc(-c4nc[nH]n4)cc32)C1] | 5-chloro-2-ethoxy-N-((1S,3R)-3-(2-(pyridin-2-yl)-6-(1H- 1,2,4-triazol-3-yl)-1H-imidazo[4,5-c]pyridin-1- yl)cyclohexyl)benzamide |
| 27 | 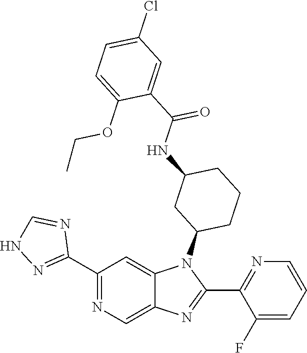 [SMILES: CCOc1ccc(Cl)cc1C(=O)N[C@H]1CCC[C@@H](n2c(-c3ncccc3F)nc3cnc(-c4nc[nH]n4)cc32)C1] | 5-chloro-2-ethoxy-N-((1S,3R)-3-(2-(3-fluoropyridin-2- yl)-6-(1H-1,2,4-triazol-3-yl)-1H-imidazo[4,5-c]pyridin-1- yl)cyclohexyl)benzamide |
| 28 | 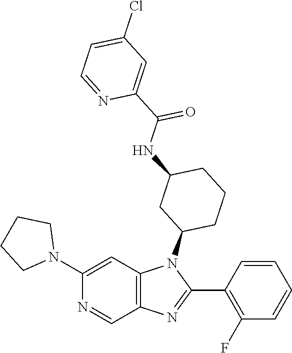 [SMILES: O=C(N[C@H]1CCC[C@@H](n2c(-c3ccccc3F)nc3cnc(N4CCCC4)cc32)C1)c1cc(Cl)ccn1] | 4-chloro-N-((1S,3R)-3-(2-(2-fluorophenyl)-6-(pyrrolidin- 1-yl)-1H-imidazo[4,5-c]pyridin-1- yl)cyclohexyl)picolinamide |
| 29 | 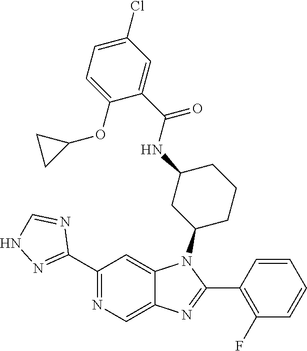 [SMILES: O=C(N[C@H]1CCC[C@@H](n2c(-c3ccccc3F)nc3cnc(-c4nc[nH]n4)cc32)C1)c1cc(Cl)ccc1OC1CC1] | 5-chloro-2-cyclopropoxy-N-((1S,3R)-3-(2-(2- fluorophenyl)-6-(1H-1,2,4-triazol-3-yl)-1H-imidazo[4,5- c]pyridin-1-yl)cyclohexyl)benzamide |
| 30 | 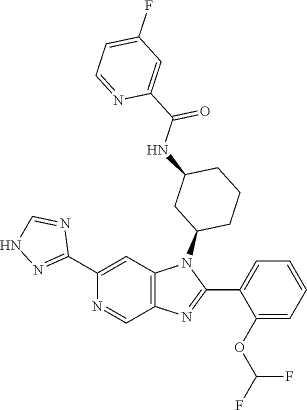 [SMILES: O=C(N[C@H]1CCC[C@@H](n2c(-c3ccccc3OC(F)F)nc3cnc(-c4nc[nH]n4)cc32)C1)c1cc(F)ccn1] | N-((1S,3R)-3-(2-(2-(difluoromethoxy)phenyl)-6-(1H- 1,2,4-triazol-3-yl)-1H-imidazo[4,5-c]pyridin-1- yl)cyclohexyl)-4-fluoropicolinamide |
| 31 | 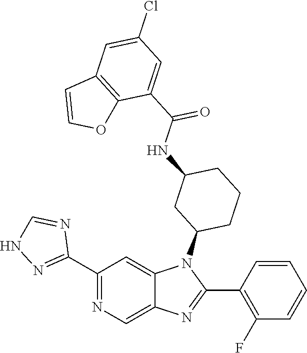 [SMILES: O=C(N[C@H]1CCC[C@@H](n2c(-c3ccccc3F)nc3cnc(-c4nc[nH]n4)cc32)C1)c1cc(Cl)cc2ccoc12] | 5-chloro-N-((1S,3R)-3-(2-(2-fluorophenyl)-6-(1H-1,2,4- triazol-3-yl)-1H-imidazo[4,5-c]pyridin-1- yl)cyclohexyl)benzofuran-7-carboxamide |
| 32 | 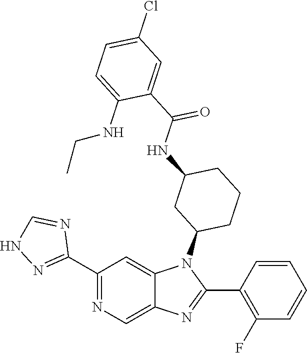 [SMILES: CCNc1ccc(Cl)cc1C(=O)N[C@H]1CCC[C@@H](n2c(-c3ccccc3F)nc3cnc(-c4nc[nH]n4)cc32)C1] | 5-chloro-2-(ethylamino)-N-((1S,3R)-3-(2-(2- fluorophenyl)-6-(1H-1,2,4-triazol-3-yl)-1H-imidazo[4,5- c]pyridin-1-yl)cyclohexyl)benzamide |
| 33 | 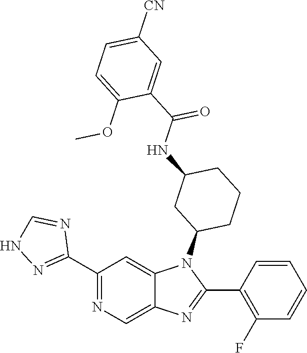 [SMILES: COc1ccc(C#N)cc1C(=O)N[C@H]1CCC[C@@H](n2c(-c3ccccc3F)nc3cnc(-c4nc[nH]n4)cc32)C1] | 5-cyano-N-((1S,3R)-3-(2-(2-fluorophenyl)-6-(1H-1,2,4- triazol-3-yl)-1H-imidazo[4,5-c]pyridin-1-yl)cyclohexyl)- 2-methoxybenzamide |
| 34 | 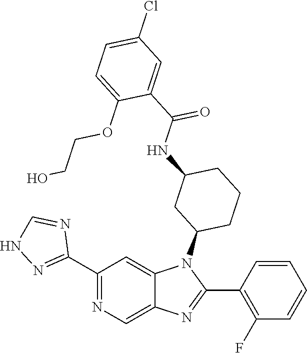 [SMILES: O=C(N[C@H]1CCC[C@@H](n2c(-c3ccccc3F)nc3cnc(-c4nc[nH]n4)cc32)C1)c1cc(Cl)ccc1OCCO] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(2-hydroxyethoxy)benzamide |
| 35 | 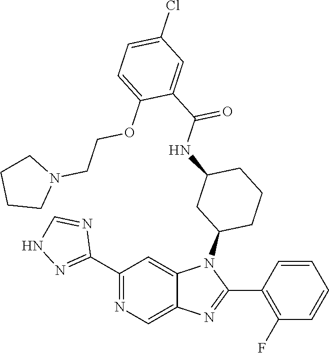 [SMILES: O=C(N[C@H]1CCC[C@@H](n2c(-c3ccccc3F)nc3cnc(-c4nc[nH]n4)cc32)C1)c1cc(Cl)ccc1OCCN1CCCC1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(2-(pyrrolidin-1-yl)ethoxy)benzamide |
| 36 | 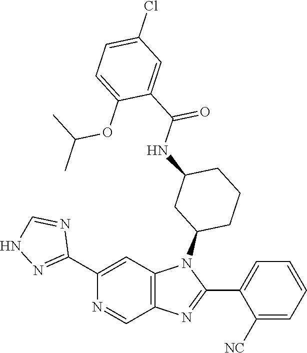 [SMILES: CC(C)Oc1ccc(Cl)cc1C(=O)N[C@H]1CCC[C@@H](n2c(-c3ccccc3C#N)nc3cnc(-c4nc[nH]n4)cc32)C1] | 5-chloro-N-((1S,3R)-3-(2-(2-cyanophenyl)-6-(1H-1,2,4- triazol-3-yl)-1H-imidazo[4,5-c]pyridin-1-yl)cyclohexyl)- 2-isopropoxybenzamide |
| 37 | 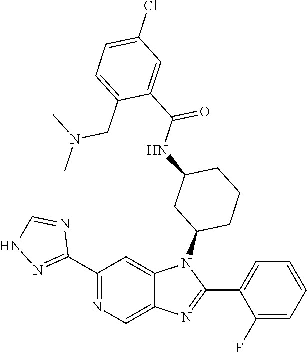 [SMILES: CN(C)Cc1ccc(Cl)cc1C(=O)N[C@H]1CCC[C@@H](n2c(-c3ccccc3F)nc3cnc(-c4nc[nH]n4)cc32)C1] | 5-chloro-2-((dimethylamino)methyl)-N-((1S,3R)-3-(2-(2- fluorophenyl)-6-(1H-1,2,4-triazol-3-yl)-1H-imidazo[4,5- c]pyridin-1-yl)cyclohexyl)benzamide |
| 38 | 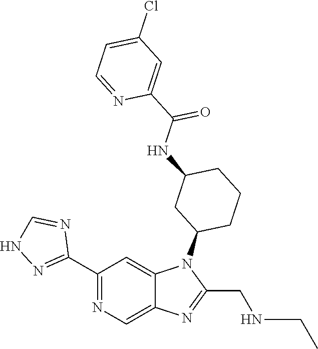 [SMILES: CCNCc1nc2cnc(-c3nc[nH]n3)cc2n1[C@@H]1CCC[C@H](NC(=O)c2cc(Cl)ccn2)C1] | 4-chloro-N-((1S,3R)-3-(2-((ethylamino)methyl)-6-(1H- 1,2,4-triazol-3-yl)-1H-imidazo[4,5-c]pyridin-1- yl)cyclohexyl)picolinamide |
| 39 | 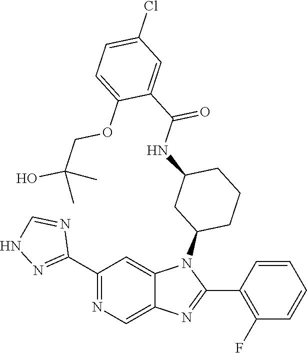 [SMILES: CC(C)(O)COc1ccc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(2-hydroxy-2-methylpropoxy)benzamide |
| 40 | 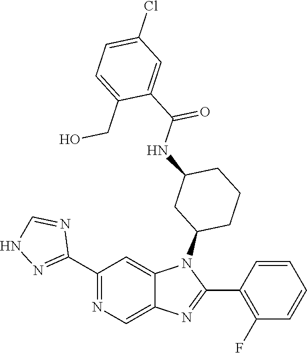 [SMILES: O=C(N[C@H]1CCC[C@@H](n2c(-c3ccccc3F)nc3cnc(-c4nc[nH]n4)cc32)C1)c1cc(Cl)ccc1CO] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(hydroxymethyl)benzamide |
| 41 | 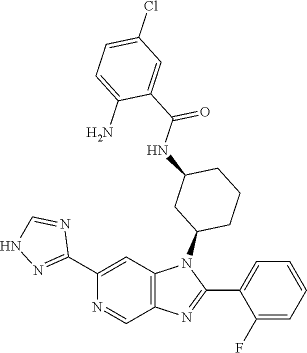 [SMILES: Nc1ccc(Cl)cc1C(=O)N[C@H]1CCC[C@@H](n2c(-c3ccccc3F)nc3cnc(-c4nc[nH]n4)cc32)C1] | 2-amino-5-chloro-N-((1S,3R)-3-(2-(2-fluorophenyl)-6- (1H-1,2,4-triazol-3-yl)-1H-imidazo[4,5-c]pyridin-1- yl)cyclohexyl)benzamide |
| 42 | 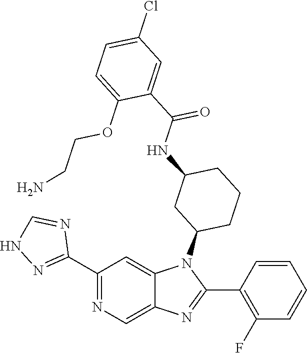 [SMILES: NCCOc1ccc(Cl)cc1C(=O)N[C@H]1CCC[C@@H](n2c(-c3ccccc3F)nc3cnc(-c4nc[nH]n4)cc32)C1] | 2-(2-aminoethoxy)-5-chloro-N-((1S,3R)-3-(2-(2- fluorophenyl)-6-(1H-1,2,4-triazol-3-yl)-1H-imidazo[4,5- c]pyridin-1-yl)cyclohexyl)benzamide |
| 43 | 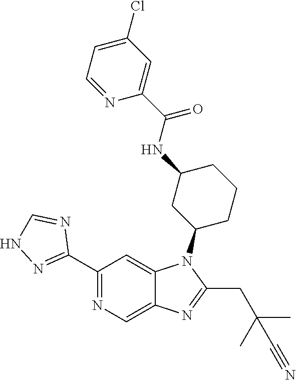 [SMILES: CC(C)(C#N)Cc1nc2cnc(-c3nc[nH]n3)cc2n1[C@@H]1CCC[C@H](NC(=O)c2cc(Cl)ccn2)C1] | 4-chloro-N-((1S,3R)-3-(2-(2-cyano-2-methylpropyl)-6- (1H-1,2,4-triazol-3-yl)-1H-imidazo[4,5-c]pyridin-1- yl)cyclohexyl)picolinamide |
| 44 | 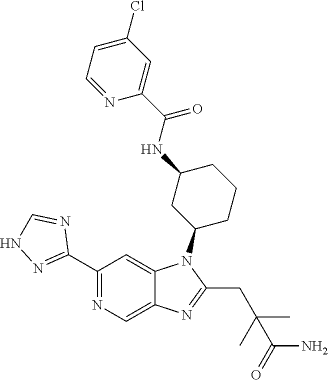 [SMILES: CC(C)(Cc1nc2cnc(-c3nc[nH]n3)cc2n1[C@@H]1CCC[C@H](NC(=O)c2cc(Cl)ccn2)C1)C(N)=O] | N-((1S,3R)-3-(2-(3-amino-2,2-dimethyl-3-oxopropyl)-6- (1H-1,2,4-triazol-3-yl)-1H-imidazo[4,5-c]pyridin-1- yl)cyclohexyl)-4-chloropicolinamide |
| 45 | 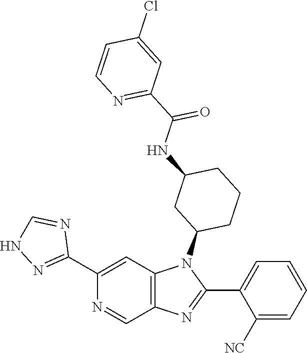 [SMILES: N#Cc1ccccc1-c1nc2cnc(-c3nc[nH]n3)cc2n1[C@@H]1CCC[C@H](NC(=O)c2cc(Cl)ccn2)C1] | 4-chloro-N-((1S,3R)-3-(2-(2-cyanophenyl)-6-(1H-1,2,4- triazol-3-yl)-1H-imidazo[4,5-c]pyridin-1- yl)cyclohexyl)picolinamide |
| 46 | 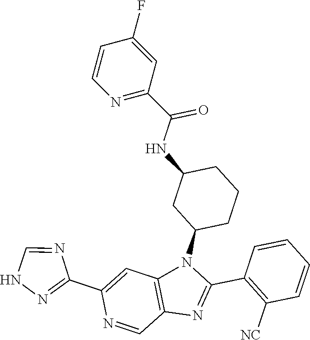 [SMILES: N#Cc1ccccc1-c1nc2cnc(-c3nc[nH]n3)cc2n1[C@@H]1CCC[C@H](NC(=O)c2cc(F)ccn2)C1] | N-((1S,3R)-3-(2-(2-cyanophenyl)-6-(1H-1,2,4-triazol-3- yl)-1H-imidazo[4,5-c]pyridin-1-yl)cyclohexyl)-4- fluoropicolinamide |
| 47 | 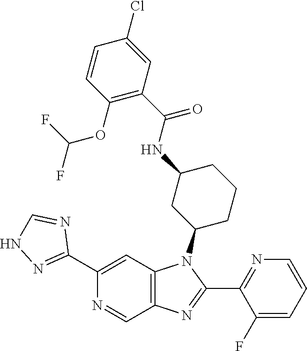 [SMILES: O=C(N[C@H]1CCC[C@@H](n2c(-c3ncccc3F)nc3cnc(-c4nc[nH]n4)cc32)C1)c1cc(Cl)ccc1OC(F)F] | 5-chloro-2-(difluoromethoxy)-N-((1S,3R)-3-(2-(3- fluoropyridin-2-yl)-6-(1H-1,2,4-triazol-3-yl)-1H- imidazo[4,5-c]pyridin-1-yl)cyclohexyl)benzamide |
| 48 | 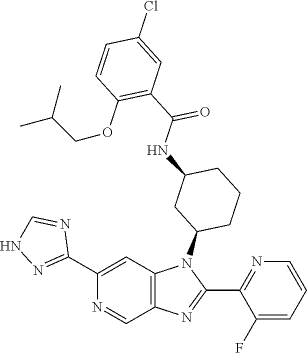 [SMILES: CC(C)COc1ccc(Cl)cc1C(=O)N[C@H]1CCC[C@@H](n2c(-c3ncccc3F)nc3cnc(-c4nc[nH]n4)cc32)C1] | 5-chloro-N-((1S,3R)-3-(2-(3-fluoropyridin-2-yl)-6-(1H- 1,2,4-triazol-3-yl)-1H-imidazo[4,5-c]pyridin-1- yl)cyclohexyl)-2-isobutoxybenzamide |
| 49 | 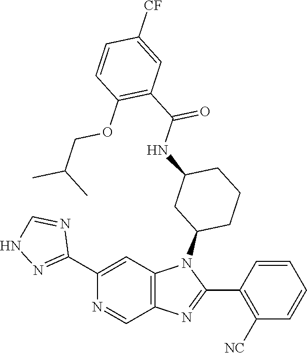 | 5-chloro-N-((1S,3R)-3-(2-(2-cyanophenyl)-6-(1H-1,2,4- triazol-3-yl)-1H-imidazo[4,5-c]pyridin-1-yl)cyclohexyl)- 2-isobutoxybenzamide |
| 50 | 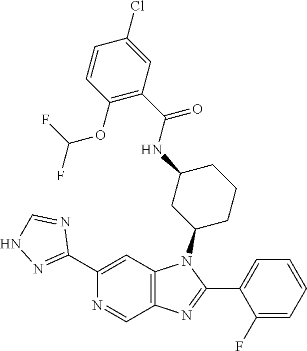 [SMILES: O=C(N[C@H]1CCC[C@@H](n2c(-c3ccccc3F)nc3cnc(-c4nc[nH]n4)cc32)C1)c1cc(Cl)ccc1OC(F)F] | 5-chloro-2-(difluoromethoxy)-N-((1S,3R)-3-(2-(2- fluorophenyl)-6-(1H-1,2,4-triazol-3-yl)-1H-imidazo[4,5- c]pyridin-1-yl)cyclohexyl)benzamide |
| 51 | 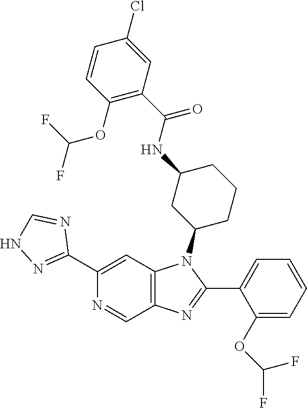 [SMILES: O=C(N[C@H]1CCC[C@@H](n2c(-c3ccccc3OC(F)F)nc3cnc(-c4nc[nH]n4)cc32)C1)c1cc(Cl)ccc1OC(F)F] | 5-chloro-2-(difluoromethoxy)-N-((1S,3R)-3-(2-(2- (difluoromethoxy)phenyl)-6-(1H-1,2,4-triazol-3-yl)-1H- imidazo[4,5-c]pyridin-1-yl)cyclohexyl)benzamide |
| 52 | 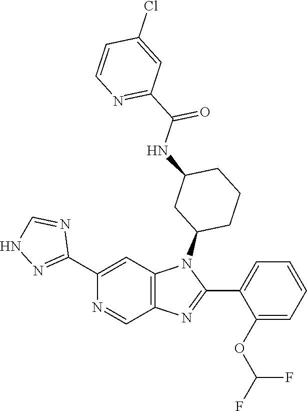 [SMILES: O=C(N[C@H]1CCC[C@@H](n2c(-c3ccccc3OC(F)F)nc3cnc(-c4nc[nH]n4)cc32)C1)c1cc(Cl)ccn1] | 4-chloro-N-((1S,3R)-3-(2-(2-(difluoromethoxy)phenyl)- 6-(1H-1,2,4-triazol-5-yl)-1H-imidazo[4,5-c]pyridin-1- yl)cyclohexyl)picolinamide |
| 53 | 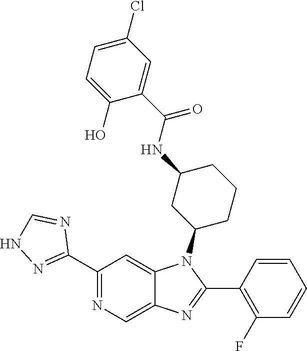 [SMILES: O=C(N[C@H]1CCC[C@@H](n2c(-c3ccccc3F)nc3cnc(-c4nc[nH]n4)cc32)C1)c1cc(Cl)ccc1O] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-hydroxybenzamide |
| 54 | 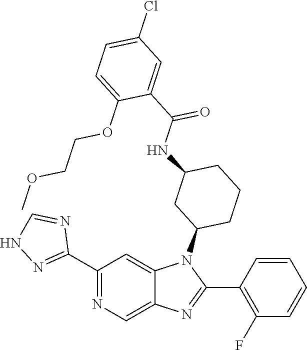 [SMILES: COCCOc1ccc(Cl)cc1C(=O)N[C@H]1CCC[C@@H](n2c(-c3ccccc3F)nc3cnc(-c4nc[nH]n4)cc32)C1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(2-methoxyethoxy)benzamide |
| 55 | 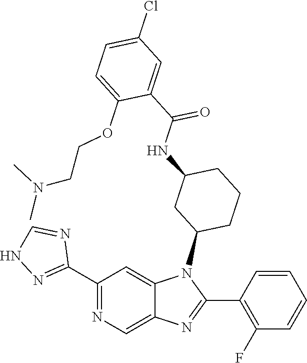 [SMILES: CN(C)CCOc1ccc(Cl)cc1C(=O)N[C@H]1CCC[C@@H](n2c(-c3ccccc3F)nc3cnc(-c4nc[nH]n4)cc32)C1] | 5-chloro-2-(2-(dimethylamino)ethoxy)-N-((1S,3R)-3-(2- (2-fluorophenyl)-6-(1H-1,2,4-triazol-3-yl)-1H- imidazo[4,5-c]pyridin-1-yl)cyclohexyl)benzamide |
| 56 | 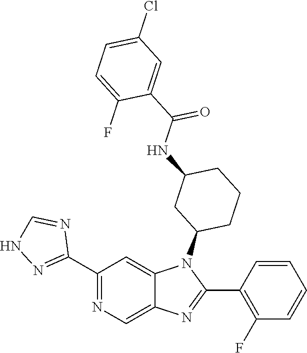 [SMILES: O=C(N[C@H]1CCC[C@@H](n2c(-c3ccccc3F)nc3cnc(-c4nc[nH]n4)cc32)C1)c1cc(Cl)ccc1F] | 5-chloro-2-fluoro-N-((1S,3R)-3-(2-(2-fluorophenyl)-6- (1H-1,2,4-triazol-3-yl)-1H-imidazo[4,5-c]pyridin-1- yl)cyclohexyl)benzamide |
| 57 | 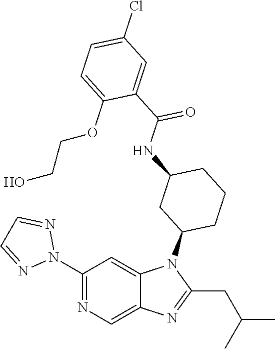 [SMILES: CC(C)Cc1nc2cnc(-n3nccn3)cc2n1[C@@H]1CCC[C@H](NC(=O)c2cc(Cl)ccc2OCCO)C1] | 5-chloro-2-(2-hydroxyethoxy)-N-((1S,3R)-3-(2-isobutyl- 6-(2H-1,2,3-triazol-2-yl)-1H-imidazo[4,5-c]pyridin-1- yl)cyclohexyl)benzamide |
| 58 | 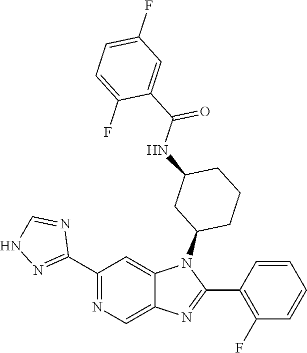 [SMILES: O=C(N[C@H]1CCC[C@@H](n2c(-c3ccccc3F)nc3cnc(-c4nc[nH]n4)cc32)C1)c1cc(F)ccc1F] | 2,5-difluoro-N-((1S,3R)-3-(2-(2-fluorophenyl)-6-(1H- 1,2,4-triazol-3-yl)-1H-imidazo[4,5-c]pyridin-1- yl)cyclohexyl)benzamide |
| 59 | 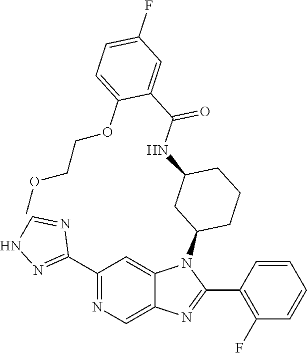 [SMILES: COCCOc1ccc(F)cc1C(=O)N[C@H]1CCC[C@@H](n2c(-c3ccccc3F)nc3cnc(-c4nc[nH]n4)cc32)C1] | 5-fluoro-N-((1S,3R)-3-(2-(2-fluorophenyl)-6-(1H-1,2,4- triazol-3-yl)-1H-imidazo[4,5-c]pyridin-1-yl)cyclohexyl)- 2-(2-methoxyethoxy)benzamide |
| 60 | 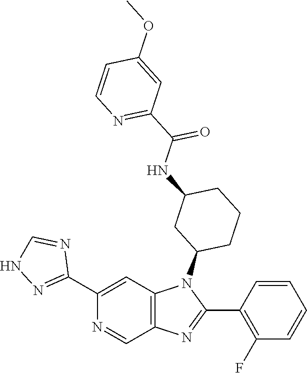 [SMILES: COc1ccnc(C(=O)N[C@H]2CCC[C@@H](n3c(-c4ccccc4F)nc4cnc(-c5nc[nH]n5)cc43)C2)c1] | N-((1S,3R)-3-(2-(2-fluorophenyl)-6-(1H-1,2,4-triazol-3- yl)-1H-imidazo[4,5-c]pyridin-1-yl)cyclohexyl)-4- methoxypicolinamide |
| 61 | 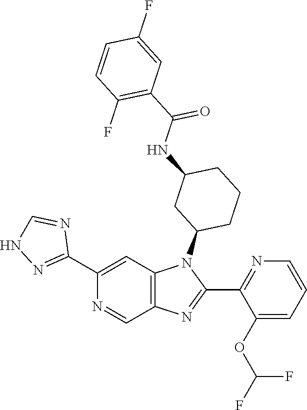 [SMILES: O=C(N[C@H]1CCC[C@@H](n2c(-c3ncccc3OC(F)F)nc3cnc(-c4nc[nH]n4)cc32)C1)c1cc(F)ccc1F] | N-((1S,3R)-3-(2-(3-(difluoromethoxy)pyridin-2-yl)-6- (1H-1,2,4-triazol-3-yl)-1H-imidazo[4,5-c]pyridin-1- yl)cyclohexyl)-2,5-difluorobenzamide |
| 62 | 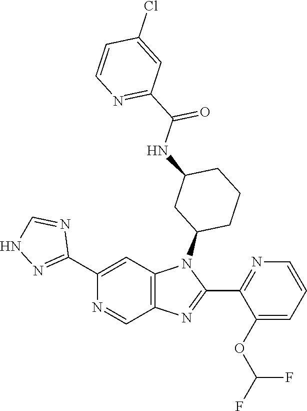 [SMILES: O=C(N[C@H]1CCC[C@@H](n2c(-c3ncccc3OC(F)F)nc3cnc(-c4nc[nH]n4)cc32)C1)c1cc(Cl)ccn1] | 4-chloro-N-((1S,3R)-3-(2-(3-(difluoromethoxy)pyridin-2- yl)-6-(1H-1,2,4-triazol-3-yl)-1H-imidazo[4,5-c]pyridin-1- yl)cyclohexyl)picolinamide |
| 63 | 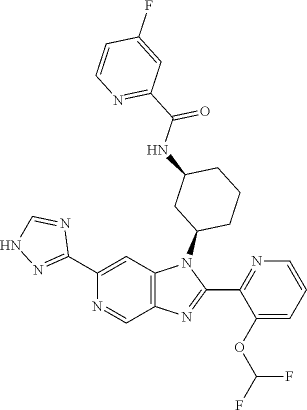 [SMILES: O=C(N[C@H]1CCC[C@@H](n2c(-c3ncccc3OC(F)F)nc3cnc(-c4nc[nH]n4)cc32)C1)c1cc(F)ccn1] | N-((1S,3R)-3-(2-(3-(difluoromethoxy)pyridin-2-yl)-6- (1H-1,2,4-triazol-3-yl)-1H-imidazo[4,5-c]pyridin-1- yl)cyclohexyl)-4-fluoropicolinamide |
| 64 | 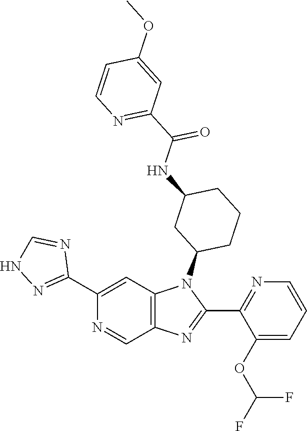 [SMILES: COc1ccnc(C(=O)N[C@H]2CCC[C@@H](n3c(-c4ncccc4OC(F)F)nc4cnc(-c5nc[nH]n5)cc43)C2)c1] | N-((1S,3R)-3-(2-(3-(difluoromethoxy)pyridin-2-yl)-6- (1H-1,2,4-triazol-3-yl)-1H-imidazo[4,5-c]pyridin-1- yl)cyclohexyl)-4-methoxypicolinamide |
| 65 | 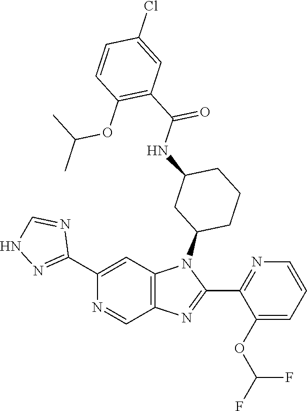 [SMILES: CC(C)Oc1ccc(Cl)cc1C(=O)N[C@H]1CCC[C@@H](n2c(-c3ncccc3OC(F)F)nc3cnc(-c4nc[nH]n4)cc32)C1] | 5-chloro-N-((1S,3R)-3-(2-(3-(difluoromethoxy)pyridin-2- yl)-6-(1H-1,2,4-triazol-3-yl)-1H-imidazo[4,5-c]pyridin-1- yl)cyclohexyl)-2-isopropoxybenzamide |
| 66 | 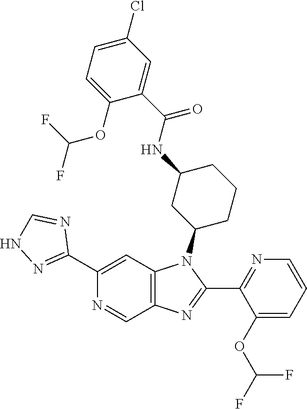 [SMILES: O=C(N[C@H]1CCC[C@@H](n2c(-c3ncccc3OC(F)F)nc3cnc(-c4nc[nH]n4)cc32)C1)c1cc(Cl)ccc1OC(F)F] | 5-chloro-2-(difluoromethoxy)-N-((1S,3R)-3-(2-(3- (difluoromethoxy)pyridin-2-yl)-6-(1H-1,2,4-triazol-3-yl)- 1H-imidazo[4,5-c]pyridin-1-yl)cyclohexyl)benzamide |
| 67 | 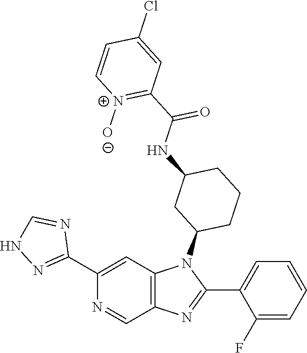 [SMILES: O=C(N[C@H]1CCC[C@@H](n2c(-c3ccccc3F)nc3cnc(-c4nc[nH]n4)cc32)C1)c1cc(Cl)cc[n+]1[O-]] | 4-chloro-2-(((1S,3R)-3-(2-(2-fluorophenyl)-6-(1H-1,2,4- triazol-3-yl)-1H-imidazo[4,5-c]pyridin-1- yl)cyclohexyl)carbamoyl)pyridine 1-oxide |
| 68 | 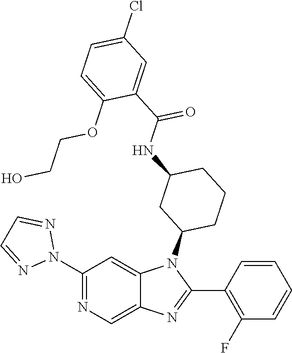 [SMILES: O=C(N[C@H]1CCC[C@@H](n2c(-c3ccccc3F)nc3cnc(-n4nccn4)cc32)C1)c1cc(Cl)ccc1OCCO] | 5-chloro-N-((1S,3R)-3-(2-(2-fluorophenyl)-6-(2H-1,2,3- triazol-2-yl)-1H-imidazo[4,5-c]pyridin-1-yl)cyclohexyl)- 2-(2-hydroxyethoxy)benzamide |
| 69 | 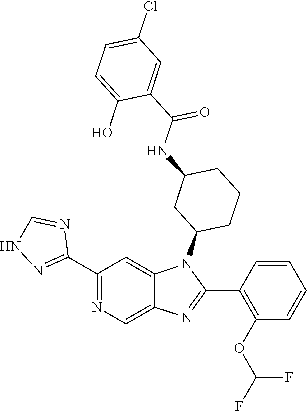 [SMILES: O=C(N[C@H]1CCC[C@@H](n2c(-c3ccccc3OC(F)F)nc3cnc(-c4nc[nH]n4)cc32)C1)c1cc(Cl)ccc1O] | 5-chloro-N-((1S,3R)-3-(2-(2-(difluoromethoxy)phenyl)- 6-(1H-1,2,4-triazol-3-yl)-1H-imidazo[4,5-c]pyridin-1- yl)cyclohexyl)-2-hydroxybenzamide |
| 70 | 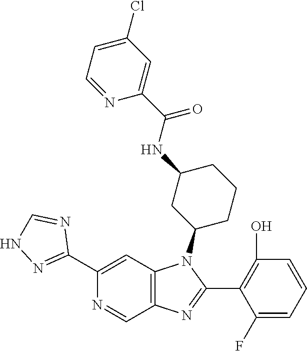 [SMILES: O=C(N[C@H]1CCC[C@@H](n2c(-c3c(O)cccc3F)nc3cnc(-c4nc[nH]n4)cc32)C1)c1cc(Cl)ccn1] | 4-chloro-N-((1S,3R)-3-(2-(2-fluoro-6-hydroxyphenyl)-6- (1H-1,2,4-triazol-3-yl)-1H-imidazo[4,5-c]pyridin-1- yl)cyclohexyl)picolinamide |
| 71 | 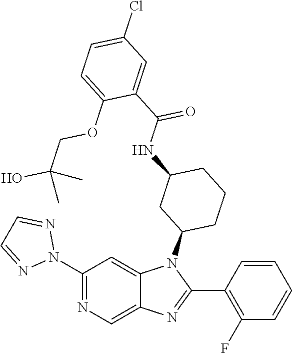 [SMILES: CC(C)(O)COc1ccc(Cl)cc1C(=O)N[C@H]1CCC[C@@H](n2c(-c3ccccc3F)nc3cnc(-n4nccn4)cc32)C1] | 5-chloro-N-((1S,3R)-3-(2-(2-fluorophenyl)-6-(2H-1,2,3- triazol-2-yl)-1H-imidazo[4,5-c]pyridin-1-yl)cyclohexyl)- 2-(2-hydroxy-2-methylpropoxy)benzamide |
| 72 | 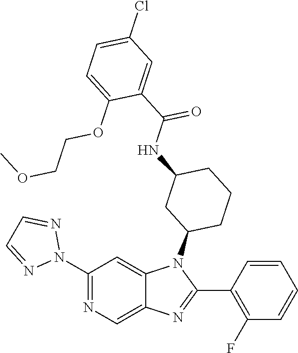 [SMILES: COCCOc1ccc(Cl)cc1C(=O)N[C@H]1CCC[C@@H](n2c(-c3ccccc3F)nc3cnc(-n4nccn4)cc32)C1] | 5-chloro-N-((1S,3R)-3-(2-(2-fluorophenyl)-6-(2H-1,2,3- triazol-2-yl)-1H-imidazo[4,5-c]pyridin-1-yl)cyclohexyl)- 2-(2-methoxyethoxy)benzamide |
| 73 | 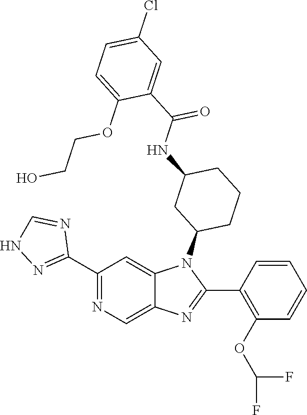 [SMILES: O=C(N[C@H]1CCC[C@@H](n2c(-c3ccccc3OC(F)F)nc3cnc(-c4nc[nH]n4)cc32)C1)c1cc(Cl)ccc1OCCO] | 5-chloro-N-((1S,3R)-3-(2-(2-(difluoromethoxy)phenyl)- 6-(1H-1,2,4-triazol-3-yl)-1H-imidazo[4,5-c]pyridin-1- yl)cyclohexyl)-2-(2-hydroxyethoxy)benzamide |
| 74 | 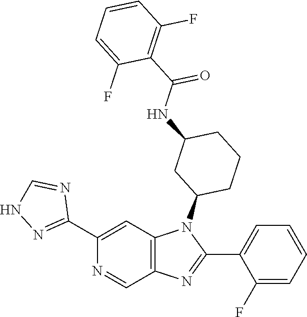 [SMILES: O=C(N[C@H]1CCC[C@@H](n2c(-c3ccccc3F)nc3cnc(-c4nc[nH]n4)cc32)C1)c1c(F)cccc1F] | 2,6-difluoro-N-((1S,3R)-3-(2-(2-fluorophenyl)-6-(1H- 1,2,4-triazol-3-yl)-1H-imidazo[4,5-c]pyridin-1- yl)cyclohexyl)benzamide |
| 75 | 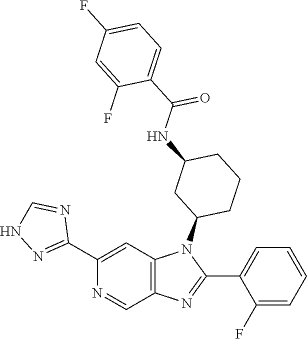 [SMILES: O=C(N[C@H]1CCC[C@@H](n2c(-c3ccccc3F)nc3cnc(-c4nc[nH]n4)cc32)C1)c1ccc(F)cc1F] | 2,4-difluoro-N-((1S,3R)-3-(2-(2-fluorophenyl)-6-(1H- 1,2,4-triazol-3-yl)-1H-imidazo[4,5-c]pyridin-1- yl)cyclohexyl)benzamide |
| 76 | 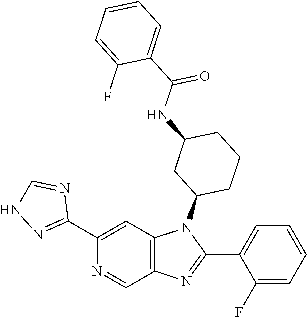 [SMILES: O=C(N[C@H]1CCC[C@@H](n2c(-c3ccccc3F)nc3cnc(-c4nc[nH]n4)cc32)C1)c1ccccc1F] | 2-fluoro-N-((1S,3R)-3-(2-(2-fluorophenyl)-6-(1H-1,2,4- triazol-3-yl)-1H-imidazo[4,5-c]pyridin-1- yl)cyclohexyl)benzamide |
| 77 | 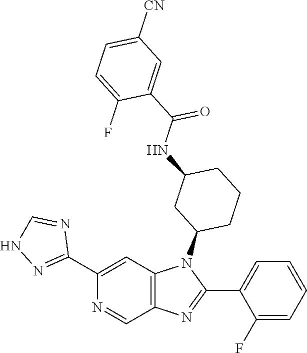 [SMILES: N#Cc1ccc(F)c(C(=O)N[C@H]2CCC[C@@H](n3c(-c4ccccc4F)nc4cnc(-c5nc[nH]n5)cc43)C2)c1] | 5-cyano-2-fluoro-N-((1S,3R)-3-(2-(2-fluorophenyl)-6- (1H-1,2,4-triazol-3-yl)-1H-imidazo[4,5-c]pyridin-1- yl)cyclohexyl)benzamide |
| 78 | 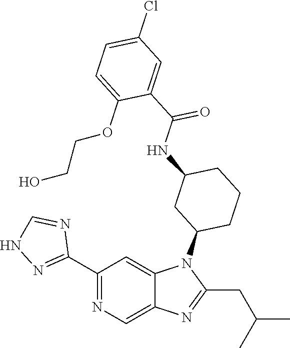 [SMILES: CC(C)Cc1nc2cnc(-c3nc[nH]n3)cc2n1[C@@H]1CCC[C@H](NC(=O)c2cc(Cl)ccc2OCCO)C1] | 5-chloro-2-(2-hydroxyethoxy)-N-((1S,3R)-3-(2-isobutyl- 6-(1H-1,2,4-triazol-3-yl)-1H-imidazo[4,5-c]pyridin-1- yl)cyclohexyl)benzamide |
| 79 | 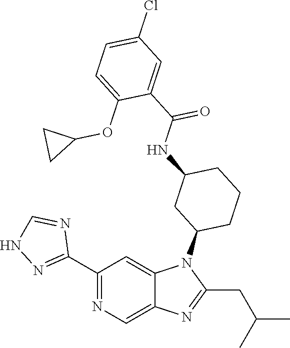 [SMILES: CC(C)Cc1nc2cnc(-c3nc[nH]n3)cc2n1[C@@H]1CCC[C@H](NC(=O)c2cc(Cl)ccc2OC2CC2)C1] | 5-chloro-2-cyclopropoxy-N-((1S,3R)-3-(2-isobutyl-6- (1H-1,2,4-triazol-3-yl)-1H-imidazo[4,5-c]pyridin-1- yl)cyclohexyl)benzamide |
| 80 | 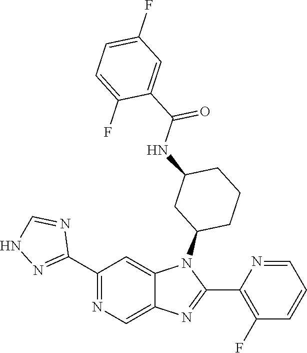 [SMILES: O=C(N[C@H]1CCC[C@@H](n2c(-c3ncccc3F)nc3cnc(-c4nc[nH]n4)cc32)C1)c1cc(F)ccc1F] | 2,5-difluoro-N-((1S,3R)-3-(2-(3-fluoropyridin-2-yl)-6- (1H-1,2,4-triazol-3-yl)-1H-imidazo[4,5-c]pyridin-1- yl)cyclohexyl)benzamide |
| 81 | 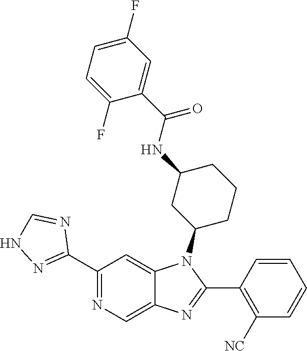 [SMILES: N#Cc1ccccc1-c1nc2cnc(-c3nc[nH]n3)cc2n1[C@@H]1CCC[C@H](NC(=O)c2cc(F)ccc2F)C1] | N-((1S,3R)-3-(2-(2-cyanophenyl)-6-(1H-1,2,4-triazol-3- yl)-1H-imidazo[4,5-c]pyridin-1-yl)cyclohexyl)-2,5- difluorobenzamide |
| 82 | 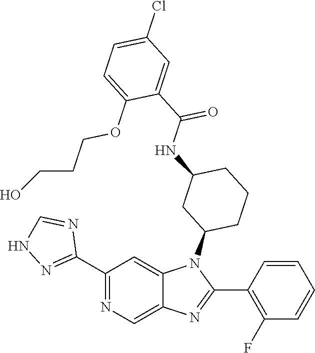 [SMILES: O=C(N[C@H]1CCC[C@@H](n2c(-c3ccccc3F)nc3cnc(-c4nc[nH]n4)cc32)C1)c1cc(Cl)ccc1OCCCO] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-(3-hydroxypropoxy)benzamide |
| 83 | 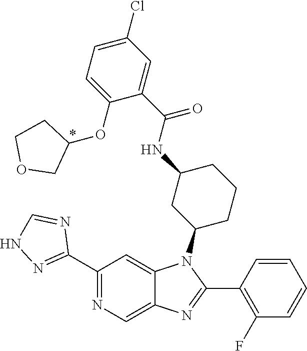 [SMILES: O=C(N[C@H]1CCC[C@@H](n2c(-c3ccccc3F)nc3cnc(-c4nc[nH]n4)cc32)C1)c1cc(Cl)ccc1OC1CCOC1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-((tetrahydrofuran-3-yl)oxy)benzamide Diastereomer #1 |
| 84 | 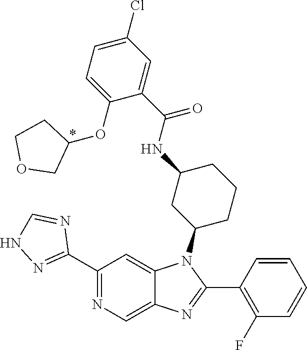 [SMILES: O=C(N[C@H]1CCC[C@@H](n2c(-c3ccccc3F)nc3cnc(-c4nc[nH]n4)cc32)C1)c1cc(Cl)ccc1OC1CCOC1] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-((tetrahydrofuran-3-yl)oxy)benzamide Diastereomer #2 |
| 85 | 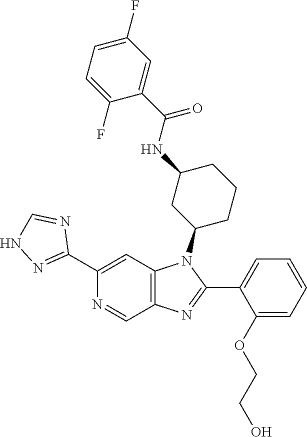 [SMILES: O=C(N[C@H]1CCC[C@@H](n2c(-c3ccccc3OCCO)nc3cnc(-c4nc[nH]n4)cc32)C1)c1cc(F)ccc1F] | 2,5-difluoro-N-((1S,3R)-3-(2-(2-(2- hydroxyethoxy)phenyl)-6-(1H-1,2,4-triazol-3-yl)-1H- imidazo[4,5-c]pyridin-1-yl)cyclohexyl)benzamide |
| 86 | 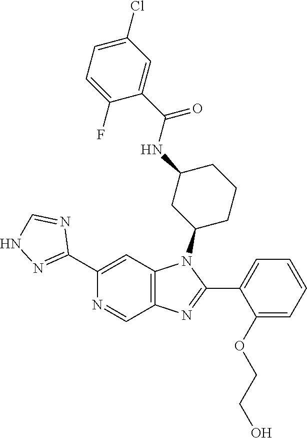 [SMILES: O=C(N[C@H]1CCC[C@@H](n2c(-c3ccccc3OCCO)nc3cnc(-c4nc[nH]n4)cc32)C1)c1cc(Cl)ccc1F] | 5-chloro-2-fluoro-N-((1S,3R)-3-(2-(2-(2- hydroxyethoxy)phenyl)-6-(1H-1,2,4-triazol-3-yl)-1H- imidazo[4,5-c]pyridin-1-yl)cyclohexyl)benzamide |
| 87 | 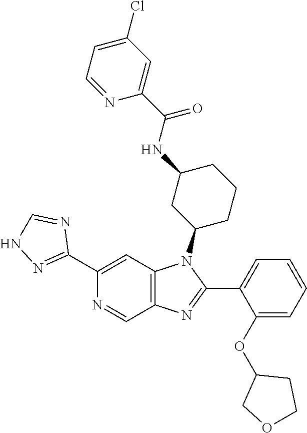 [SMILES: O=C(N[C@H]1CCC[C@@H](n2c(-c3ccccc3OC3CCOC3)nc3cnc(-c4nc[nH]n4)cc32)C1)c1cc(Cl)ccn1] | 4-chloro-N-((1S,3R)-3-(2-(2-((tetrahydrofuran-3- yl)oxy)phenyl)-6-(1H-1,2,4-triazol-3-yl)-1H-imidazo[4,5- c]pyridin-1-yl)cyclohexyl)picolinamide |
| 88 | 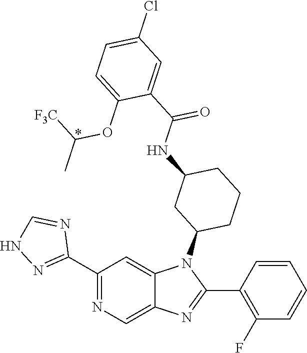 [SMILES: CC(Oc1ccc(Cl)cc1C(=O)N[C@H]1CCC[C@@H](n2c(-c3ccccc3F)nc3cnc(-c4nc[nH]n4)cc32)C1)C(F)(F)F] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-((1,1,1-trifluoropropan-2-yl)oxy)benzamide Diastereomer #1 |
| 89 | 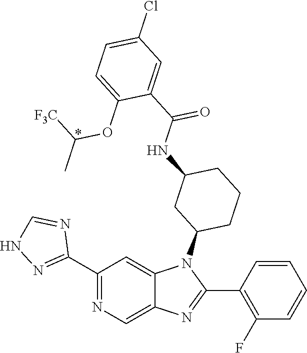 [SMILES: CC(Oc1ccc(Cl)cc1C(=O)N[C@H]1CCC[C@@H](n2c(-c3ccccc3F)nc3cnc(-c4nc[nH]n4)cc32)C1)C(F)(F)F] | 5-chloro-N-((1S,3R)-3-(2-(2-fluorophenyl)-6-(1H-1,2,4- triazol-3-yl)-1H-imidazo[4,5-c]pyridin-1-yl)cyclohexyl)- 2-((1,1,1-trifluoropropan-2-yl)oxy)benzamide Diastereomer #2 |
| 90 | 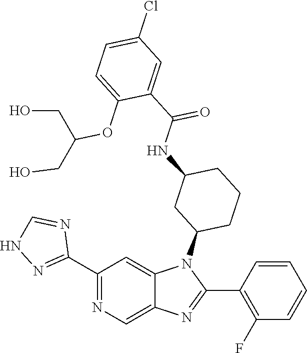 [SMILES: O=C(N[C@H]1CCC[C@@H](n2c(-c3ccccc3F)nc3cnc(-c4nc[nH]n4)cc32)C1)c1cc(Cl)ccc1OC(CO)CO] | 5-chloro-2-((1,3-dihydroxypropan-2-yl)oxy)-N-((1S,3R)- 3-(2-(2-fluorophenyl)-6-(1H-1,2,4-triazol-3-yl)-1H- imidazo[4,5-c]pyridin-1-yl)cyclohexyl)benzamide |
| 91 | 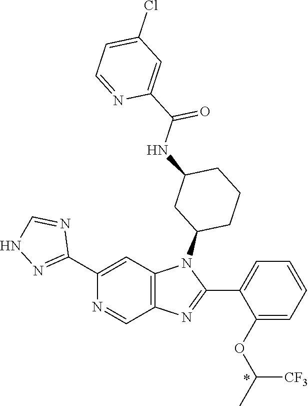 [SMILES: CC(Oc1ccccc1-c1nc2cnc(-c3nc[nH]n3)cc2n1[C@@H]1CCC[C@H](NC(=O)c2cc(Cl)ccn2)C1)C(F)(F)F] | N-((1S,3R)-3-(6-(1H-1,2,4-triazol-3-yl)-2-(2-((1,1,1- trifluoropropan-2-yl)oxy)phenyl)-1H-imidazo[4,5- c]pyridin-1-yl)cyclohexyl)-4-chloropicolinamide Diastereomer #1 |
| 92 | 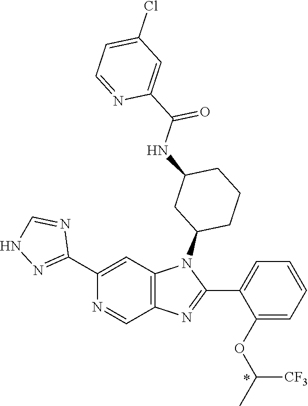 [SMILES: CC(Oc1ccccc1-c1nc2cnc(-c3nc[nH]n3)cc2n1[C@@H]1CCC[C@H](NC(=O)c2cc(Cl)ccn2)C1)C(F)(F)F] | N-((1S,3R)-3-(6-(1H-1,2,4-triazol-3-yl)-2-(2-((1,1,1- trifluoropropan-2-yl)oxy)phenyl)-1H-imidazo[4,5- c]pyridin-1-yl)cyclohexyl)-4-chloropicolinamide Diastereomer #2 |
| 93 | 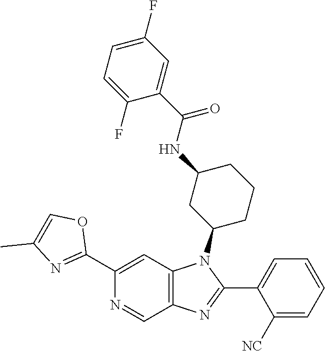 [SMILES: Cc1coc(-c2cc3c(cn2)nc(-c2ccccc2C#N)n3[C@@H]2CCC[C@H](NC(=O)c3cc(F)ccc3F)C2)n1] | N-((1S,3R)-3-(2-(2-cyanophenyl)-6-(4-methyloxazol-2- yl)-1H-imidazo[4,5-c]pyridin-1-yl)cyclohexyl)-2,5- difluorobenzamide |
| 94 | 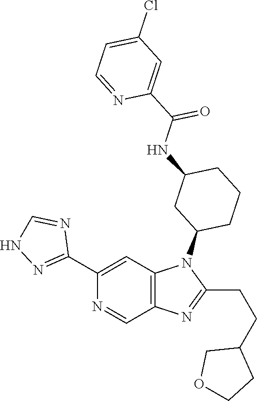 [SMILES: O=C(N[C@H]1CCC[C@@H](n2c(CCC3CCOC3)nc3cnc(-c4nc[nH]n4)cc32)C1)c1cc(Cl)ccn1] | 4-chloro-N-((1S,3R)-3-(2-(2-(tetrahydrofuran-3-yl)ethyl)- 6-(1H-1,2,4-triazol-3-yl)-1H-imidazo[4,5-c]pyridin-1- yl)cyclohexyl)picolinamide |
| 95 | 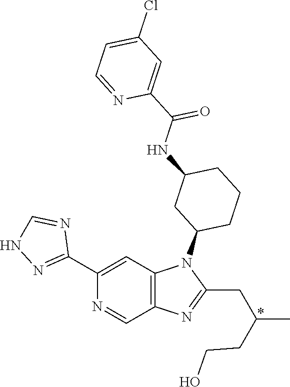 [SMILES: CC(CCO)Cc1nc2cnc(-c3nc[nH]n3)cc2n1[C@@H]1CCC[C@H](NC(=O)c2cc(Cl)ccn2)C1] | 4-chloro-N-((1S,3R)-3-(2-(4-hydroxy-2-methylbutyl)-6- (1H-1,2,4-triazol-3-yl)-1H-imidazo[4,5-c]pyridin-1- yl)cyclohexyl)picolinamide Diastereomer #1 |
| 96 | 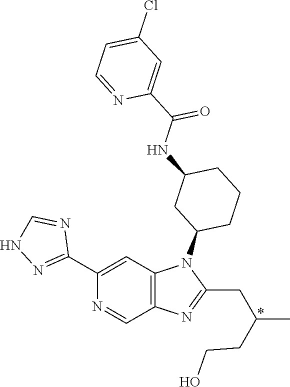 [SMILES: CC(CCO)Cc1nc2cnc(-c3nc[nH]n3)cc2n1[C@@H]1CCC[C@H](NC(=O)c2cc(Cl)ccn2)C1] | 4-chloro-N-((1S,3R)-3-(2-(4-hydroxy-2-methylbutyl)-6- (1H-1,2,4-triazol-3-yl)-1H-imidazo[4,5-c]pyridin-1- yl)cyclohexyl)picolinamide Diastereomer #2 |
| 97 | 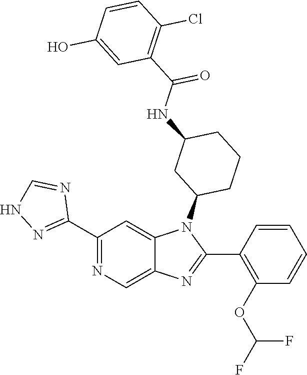 [SMILES: O=C(N[C@H]1CCC[C@@H](n2c(-c3ccccc3OC(F)F)nc3cnc(-c4nc[nH]n4)cc32)C1)c1cc(O)ccc1Cl] | 2-chloro-N-((1S,3R)-3-(2-(3-(difluoromethoxy)pyridin-2- yl)-6-(1H-1,2,4-triazol-3-yl)-1H-imidazo[4,5-c]pyridin-1- yl)cyclohexyl)-5-hydroxybenzamide |
| 98 | 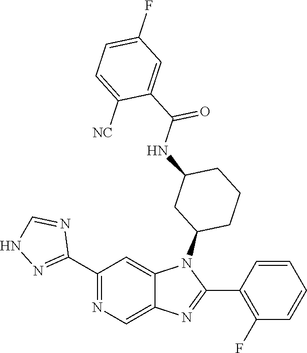 [SMILES: N#Cc1ccc(F)cc1C(=O)N[C@H]1CCC[C@@H](n2c(-c3ccccc3F)nc3cnc(-c4nc[nH]n4)cc32)C1] | 2-cyano-5-fluoro-N-((1S,3R)-3-(2-(2-fluorophenyl)-6- (4H-1,2,4-triazol-3-yl)-1H-imidazo[4,5-c]pyridin-1- yl)cyclohexyl)benzamide |
| 99 | 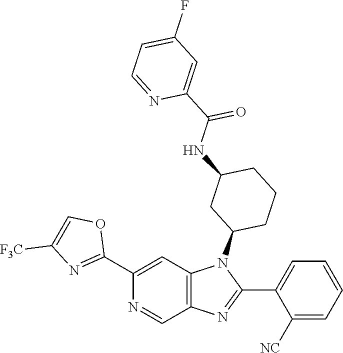 [SMILES: N#Cc1ccccc1-c1nc2cnc(-c3nc(C(F)(F)F)co3)cc2n1[C@@H]1CCC[C@H](NC(=O)c2cc(F)ccn2)C1] | N-((1S,3R)-3-(2-(2-cyanophenyl)-6-(4- (trifluoromethyl)oxazol-2-yl)-1H-imidazo[4,5-c]pyridin- 1-yl)cyclohexyl)-4-fluoropicolinamide |
| 100 | 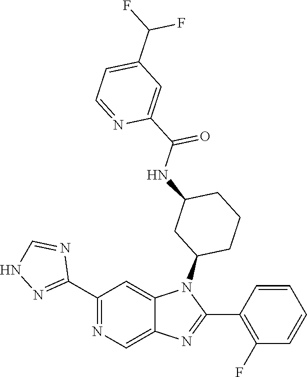 [SMILES: O=C(N[C@H]1CCC[C@@H](n2c(-c3ccccc3F)nc3cnc(-c4nc[nH]n4)cc32)C1)c1cc(C(F)F)ccn1] | 4-(difluoromethyl)-N-((1S,3R)-3-(2-(2-fluorophenyl)-6- (1H-1,2,4-triazol-3-yl)-1H-imidazo[4,5-c]pyridin-1- yl)cyclohexyl)picolinamide |
| 101 | 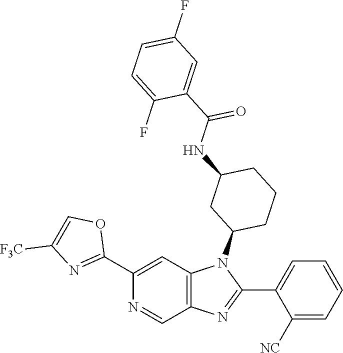 [SMILES: N#Cc1ccccc1-c1nc2cnc(-c3nc(C(F)(F)F)co3)cc2n1[C@@H]1CCC[C@H](NC(=O)c2cc(F)ccc2F)C1] | N-((1S,3R)-3-(2-(2-cyanophenyl)-6-(4- (trifluoromethyl)oxazol-2-yl)-1H-imidazo[4,5-c]pyridin- 1-yl)cyclohexyl)-2,5-difluorobenzamide |
| 102 | 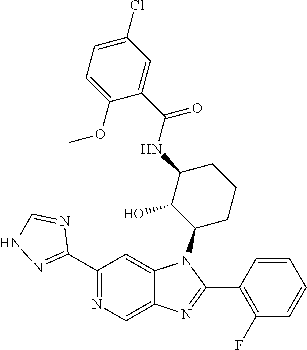 [SMILES: COc1ccc(Cl)cc1C(=O)N[C@H]1CCC[C@@H](n2c(-c3ccccc3F)nc3cnc(-c4nc[nH]n4)cc32)[C@@H]1O] | 5-chloro-N-((1S,2R,3R)-3-(2-(2-fluorophenyl)-6-(1H- 1,2,4-triazol-3-yl)-1H-imidazo[4,5-c]pyridin-1-yl)-2- hydroxycyclohexyl)-2-methoxybenzamide |
| 103 | 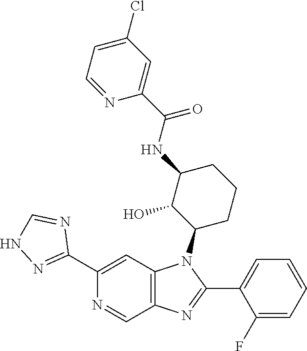 [SMILES: O=C(N[C@H]1CCC[C@@H](n2c(-c3ccccc3F)nc3cnc(-c4nc[nH]n4)cc32)[C@@H]1O)c1cc(Cl)ccn1] | 4-chloro-N-((1S,2R,3R)-3-(2-(2-fluorophenyl)-6-(1H- 1,2,4-triazol-3-yl)-1H-imidazo[4,5-c]pyridin-1-yl)-2- hydroxycyclohexyl)picolinamide |
| 104 | 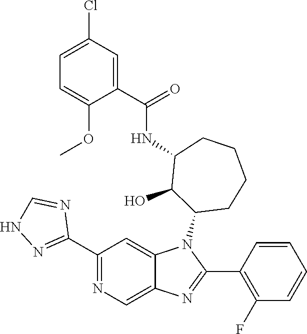 [SMILES: COc1ccc(Cl)cc1C(=O)N[C@@H]1CCCC[C@H](n2c(-c3ccccc3F)nc3cnc(-c4nc[nH]n4)cc32)[C@H]1O] | 5-chloro-N-((1R,2S,3S)-3-(2-(2-fluorophenyl)-6-(1H- 1,2,4-triazol-3-yl)-1H-imidazo[4,5-c]pyridin-1-yl)-2- hydroxycycloheptyl)-2-methoxybenzamide |
| 105 | 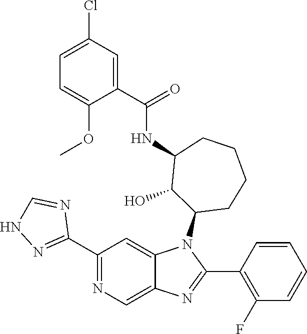 [SMILES: COc1ccc(Cl)cc1C(=O)N[C@H]1CCCC[C@@H](n2c(-c3ccccc3F)nc3cnc(-c4nc[nH]n4)cc32)[C@@H]1O] | 5-chloro-N-((1S,2R,3R)-3-(2-(2-fluorophenyl)-6-(1H- 1,2,4-triazol-3-yl)-1H-imidazo[4,5-c]pyridin-1-yl)-2- hydroxycycloheptyl)-2-methoxybenzamide |
| 106 | 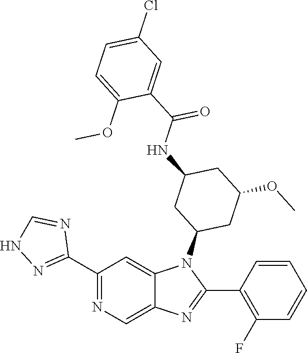 [SMILES: COc1ccc(Cl)cc1C(=O)N[C@H]1C[C@H](OC)C[C@@H](n2c(-c3ccccc3F)nc3cnc(-c4nc[nH]n4)cc32)C1] | 5-chloro-N-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)-2-methoxybenzamide |
| 107 | 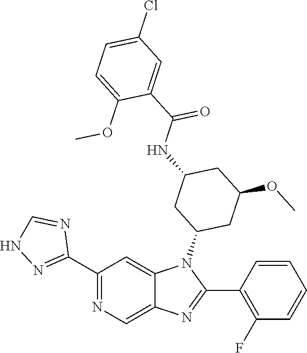 [SMILES: COc1ccc(Cl)cc1C(=O)N[C@@H]1C[C@@H](OC)C[C@H](n2c(-c3ccccc3F)nc3cnc(-c4nc[nH]n4)cc32)C1] | 5-chloro-N-((1S,3R,5R)-3-(2-(2-fluorophenyl)-6-(1H- 1,2,4-triazol-3-yl)-1H-imidazo[4,5-c]pyridin-1-yl)-5- methoxycyclohexyl)-2-methoxybenzamide |
| 108 | 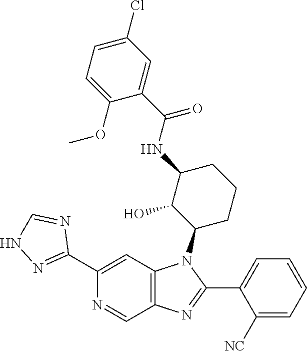 [SMILES: COc1ccc(Cl)cc1C(=O)N[C@H]1CCC[C@@H](n2c(-c3ccccc3C#N)nc3cnc(-c4nc[nH]n4)cc32)[C@@H]1O] | 5-chloro-N-((1S,2R,3R)-3-(2-(2-cyanophenyl)-6-(1H- 1,2,4-triazol-3-yl)-1H-imidazo[4,5-c]pyridin-1-yl)-2- hydroxycyclohexyl)-2-methoxybenzamide |
| 109 | 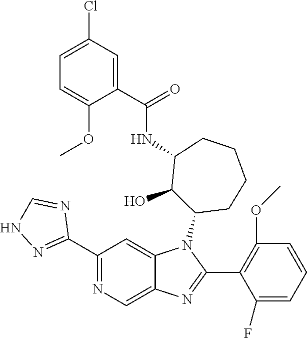 [SMILES: COc1ccc(Cl)cc1C(=O)N[C@@H]1CCCC[C@H](n2c(-c3c(F)cccc3OC)nc3cnc(-c4nc[nH]n4)cc32)[C@H]1O] | 5-chloro-N-((1R,2S,3S)-3-(2-(2-fluoro-6- methoxyphenyl)-6-(1H-1,2,4-triazol-3-yl)-1H- imidazo[4,5-c]pyridin-1-yl)-2-hydroxycycloheptyl)-2- methoxybenzamide |
| 110 | 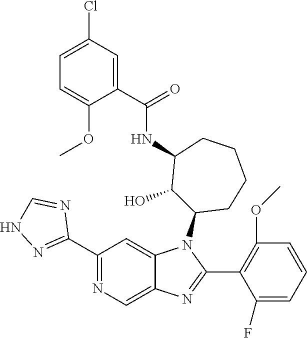 [SMILES: COc1ccc(Cl)cc1C(=O)N[C@H]1CCCC[C@@H](n2c(-c3c(F)cccc3OC)nc3cnc(-c4nc[nH]n4)cc32)[C@@H]1O] | 5-chloro-N-((1S,2R,3R)-3-(2-(2-fluoro-6- methoxyphenyl)-6-(1H-1,2,4-triazol-3-yl)-1H- imidazo[4,5-c]pyridin-1-yl)-2-hydroxycycloheptyl)-2- methoxybenzamide |
| 111 | 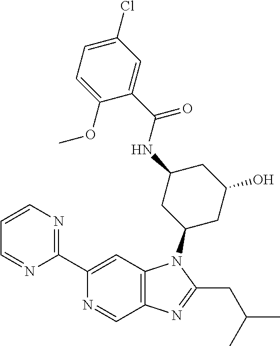 [SMILES: COc1ccc(Cl)cc1C(=O)N[C@H]1C[C@H](O)C[C@@H](n2c(CC(C)C)nc3cnc(-c4ncccn4)cc32)C1] | 5-chloro-N-((1R,3S,5S)-3-hydroxy-5-(2-isobutyl-6- (pyrimidin-2-yl)-1H-imidazo[4,5-c]pyridin-1- yl)cyclohexyl)-2-methoxybenzamide |
| 112 | 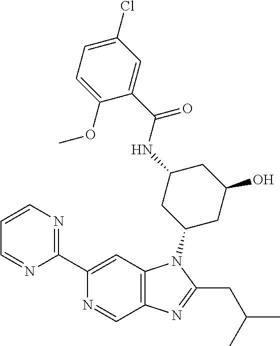 [SMILES: COc1ccc(Cl)cc1C(=O)N[C@@H]1C[C@@H](O)C[C@H](n2c(CC(C)C)nc3cnc(-c4ncccn4)cc32)C1] | 5-chloro-N-((1S,3R,5R)-3-hydroxy-5-(2-isobutyl-6- (pyrimidin-2-yl)-1H-imidazo[4,5-c]pyridin-1- yl)cyclohexyl)-2-methoxybenzamide |
| 113 | 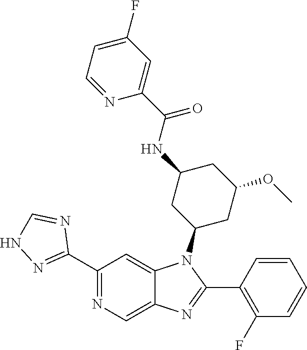 [SMILES: CO[C@H]1C[C@H](NC(=O)c2cc(F)ccn2)C[C@H](n2c(-c3ccccc3F)nc3cnc(-c4nc[nH]n4)cc32)C1] | 4-fluoro-N-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)picolinamide |
| 114 | 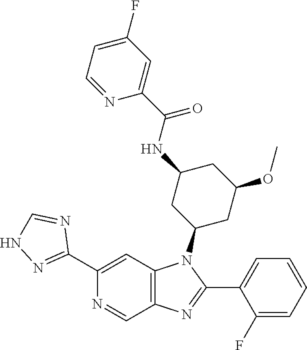 [SMILES: CO[C@@H]1C[C@H](NC(=O)c2cc(F)ccn2)C[C@H](n2c(-c3ccccc3F)nc3cnc(-c4nc[nH]n4)cc32)C1] | 4-fluoro-N-((1R,3S,5R)-3-(2-(2-fluorophenyl)-6-(1H- 1,2,4-triazol-3-yl)-1H-imidazo[4,5-c]pyridin-1-yl)-5- methoxycyclohexyl)picolinamide |
| 115 | 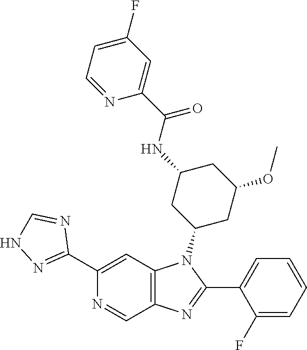 [SMILES: CO[C@H]1C[C@@H](NC(=O)c2cc(F)ccn2)C[C@@H](n2c(-c3ccccc3F)nc3cnc(-c4nc[nH]n4)cc32)C1] | 4-fluoro-N-((1S,3R,5S)-3-(2-(2-fluorophenyl)-6-(1H- 1,2,4-triazol-3-yl)-1H-imidazo[4,5-c]pyridin-1-yl)-5- methoxycyclohexyl)picolinamide |
| 116 | 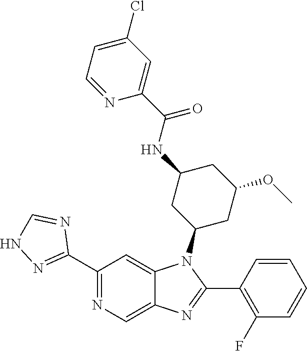 [SMILES: CO[C@H]1C[C@H](NC(=O)c2cc(Cl)ccn2)C[C@H](n2c(-c3ccccc3F)nc3cnc(-c4nc[nH]n4)cc32)C1] | 4-chloro-N-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)picolinamide |
| 117 | 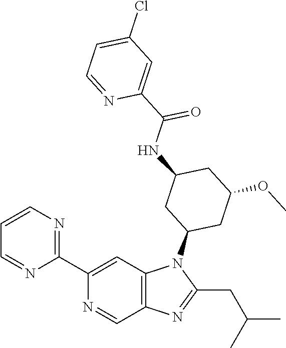 [SMILES: CO[C@H]1C[C@H](NC(=O)c2cc(Cl)ccn2)C[C@H](n2c(CC(C)C)nc3cnc(-c4ncccn4)cc32)C1] | 4-chloro-N-((1R,3S,5S)-3-(2-isobutyl-6-(pyrimidin-2-yl)- 1H-imidazo[4,5-c]pyridin-1-yl)-5- methoxycyclohexyl)picolinamide |
| 118 | 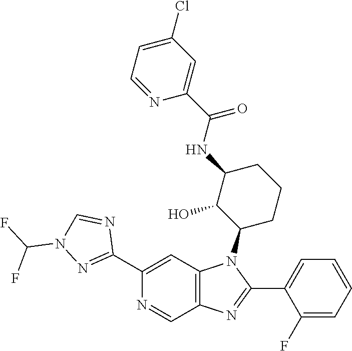 [SMILES: O=C(N[C@H]1CCC[C@@H](n2c(-c3ccccc3F)nc3cnc(-c4ncn(C(F)F)n4)cc32)[C@@H]1O)c1cc(Cl)ccn1] | 4-chloro-N-((1S,2R,3R)-3-(6-(1-(difluoromethyl)-1H- 1,2,4-triazol-3-yl)-2-(2-fluorophenyl)-1H-imidazo[4,5- c]pyridin-1-yl)-2-hydroxycyclohexyl)picolinamide |
| 119 | 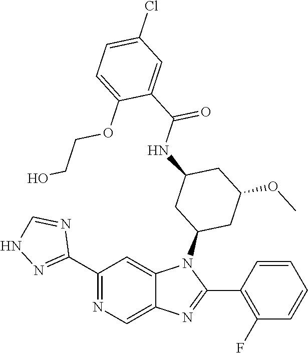 [SMILES: CO[C@H]1C[C@H](NC(=O)c2cc(Cl)ccc2OCCO)C[C@H](n2c(-c3ccccc3F)nc3cnc(-c4nc[nH]n4)cc32)C1] | 5-chloro-N-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)-2-(2-hydroxyethoxy)benzamide |
| 120 | 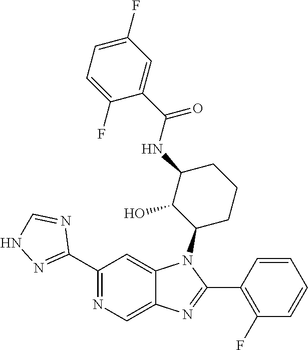 [SMILES: O=C(N[C@H]1CCC[C@@H](n2c(-c3ccccc3F)nc3cnc(-c4nc[nH]n4)cc32)[C@@H]1O)c1cc(F)ccc1F] | 2,5-difluoro-N-((1S,2R,3R)-3-(2-(2-fluorophenyl)-6-(1H- 1,2,4-triazol-3-yl)-1H-imidazo[4,5-c]pyridin-1-yl)-2- hydroxycyclohexyl)benzamide |
| 121 | 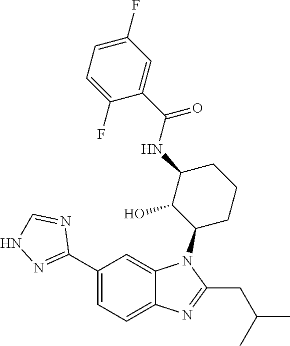 [SMILES: CC(C)Cc1nc2ccc(-c3nc[nH]n3)cc2n1[C@@H]1CCC[C@H](NC(=O)c2cc(F)ccc2F)[C@H]1O] | 2,5-difluoro-N-((1S,2R,3R)-2-hydroxy-3-(2-isobutyl-6- (1H-1,2,4-triazol-3-yl)-1H-imidazo[4,5-c]pyridin-1- yl)cyclohexyl)benzamide |
| 122 | 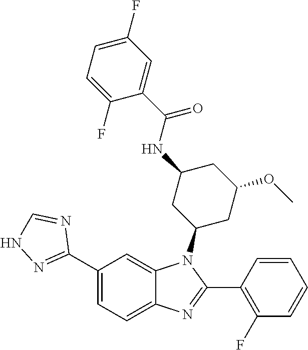 [SMILES: CO[C@H]1C[C@H](NC(=O)c2cc(F)ccc2F)C[C@H](n2c(-c3ccccc3F)nc3ccc(-c4nc[nH]n4)cc32)C1] | 2,5-difluoro-N-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)benzamide |
| 123 | 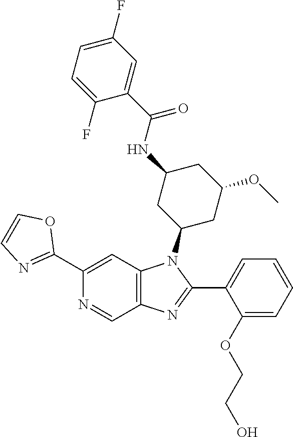 [SMILES: CO[C@H]1C[C@H](NC(=O)c2cc(F)ccc2F)C[C@H](n2c(-c3ccccc3OCCO)nc3cnc(-c4ncco4)cc32)C1] | 2,5-difluoro-N-((1R,3S,5S)-3-(2-(2-(2- hydroxyethoxy)phenyl)-6-(oxazol-2-yl)-1H-imidazo[4,5- c]pyridin-1-yl)-5-methoxycyclohexyl)benzamide |
| 124 | 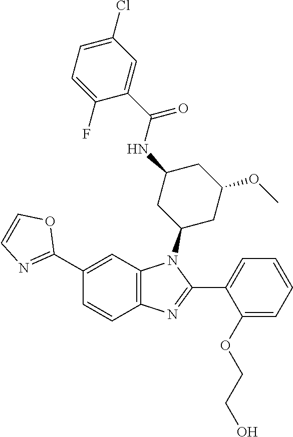 [SMILES: CO[C@H]1C[C@H](NC(=O)c2cc(Cl)ccc2F)C[C@H](n2c(-c3ccccc3OCCO)nc3ccc(-c4ncco4)cc32)C1] | 5-chloro-2-fluoro-N-((1R,3S,5S)-3-(2-(2-(2- hydroxyethoxy)phenyl)-6-(oxazol-2-yl)-1H-imidazo[4,5- c]pyridin-1-yl)-5-methoxycyclohexyl)benzamide |
| 125 | 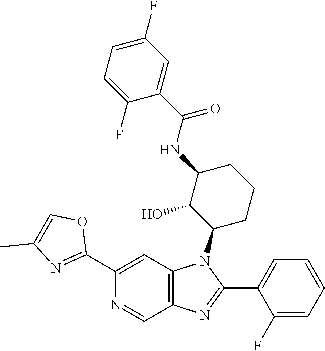 [SMILES: Cc1coc(-c2cc3c(cn2)nc(-c2ccccc2F)n3[C@@H]2CCC[C@H](NC(=O)c3cc(F)ccc3F)[C@H]2O)n1] | 2,5-difluoro-N-((1S,2R,3R)-3-(2-(2-fluorophenyl)-6-(4- methyloxazol-2-yl)-1H-imidazo[4,5-c]pyridin-1-yl)-2- hydroxycyclohexyl)benzamide |
| 126 | 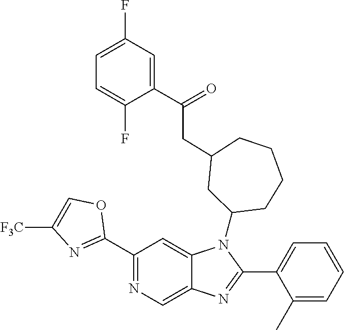 [SMILES: Cc1ccccc1-c1nc2cnc(-c3nc(C(F)(F)F)co3)cc2n1C1CCCCC(CC(=O)c2cc(F)ccc2F)C1] | 2,5-difluoro-N-(3-(2-(2-fluorophenyl)-6-(4- (trifluoromethyl)oxazol-2-yl)-1H-imidazo[4,5-c]pyridin- 1-yl)-2-hydroxycycloheptyl)benzamide⁽¹⁾ ⁽¹⁾ relative stereochemistry at centers 1 and 3 of the cycloheptyl ring is cis with —OH at center 2 trans |
| 127 | 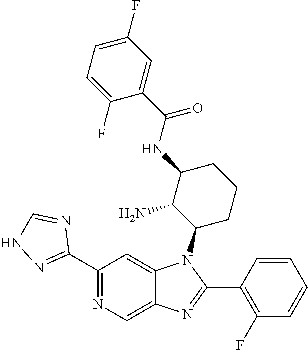 [SMILES: N[C@@H]1[C@@H](NC(=O)c2cc(F)ccc2F)CCC[C@H]1n1c(-c2ccccc2F)nc2cnc(-c3nc[nH]n3)cc21] | N-((1S,2R,3R)-2-amino-3-(2-(2-fluorophenyl)-6-(1H- 1,2,4-triazol-3-yl)-1H-imidazo[4,5-c]pyridin-1- yl)cyclohexyl)-2,5-difluorobenzamide |
| 128 | 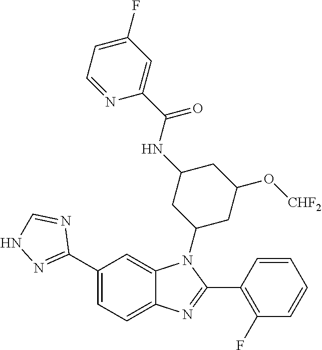 [SMILES: O=C(NC1CC(OC(F)F)CC(n2c(-c3ccccc3F)nc3ccc(-c4nc[nH]n4)cc32)C1)c1cc(F)ccn1] | N-(3-(difluoromethoxy)-5-(2-(2-fluorophenyl)-6-(1H- 1,2,4-triazol-3-yl)-1H-imidazo[4,5-c]pyridin-1- yl)cyclohexyl)-4-fluoropicolinamide⁽²⁾ ⁽²⁾ relative stereochemistry at centers 1 and 5 of the cyclohexyl ring is cis with —OCHF₂ at center 3 trans |
| 129 | 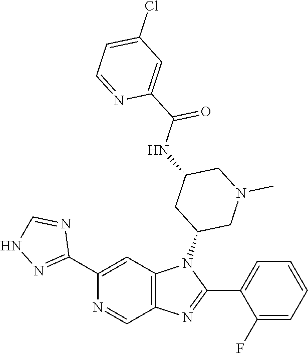 [SMILES: CN1C[C@@H](NC(=O)c2cc(Cl)ccn2)C[C@@H](n2c(-c3ccccc3F)nc3cnc(-c4nc[nH]n4)cc32)C1] | 4-chloro-N-((3S,5R)-5-(2-(2-fluorophenyl)-6-(1H-1,2,4- triazol-3-yl)-1H-imidazo[4,5-c]pyridin-1-yl)-1- methylpiperidin-3-yl)picolinamide |
| 130 | 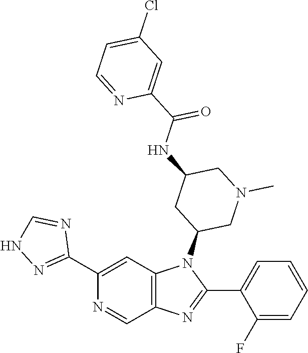 [SMILES: CN1C[C@H](NC(=O)c2cc(Cl)ccn2)C[C@H](n2c(-c3ccccc3F)nc3cnc(-c4nc[nH]n4)cc32)C1] | 4-chloro-N-((3R,5S)-5-(2-(2-fluorophenyl)-6-(1H-1,2,4- triazol-3-yl)-1H-imidazo[4,5-c]pyridin-1-yl)-1- methylpiperidin-3-yl)picolinamide |
| 131 | 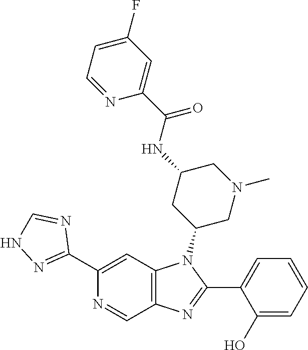 [SMILES: CN1C[C@@H](NC(=O)c2cc(F)ccn2)C[C@@H](n2c(-c3ccccc3O)nc3cnc(-c4nc[nH]n4)cc32)C1] | 4-chloro-N-((3S,5R)-5-(2-(3-fluoropyridin-2-yl)-6-(1H- 1,2,4-triazol-3-yl)-1H-imidazo[4,5-c]pyridin-1-yl)-1- methylpiperidin-3-yl)picolinamide |
| 132 | 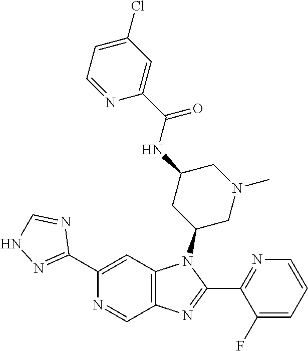 [SMILES: CN1C[C@H](NC(=O)c2cc(Cl)ccn2)C[C@H](n2c(-c3ncccc3F)nc3cnc(-c4nc[nH]n4)cc32)C1] | 4-chloro-N-((3R,5S)-5-(2-(3-fluoropyridin-2-yl)-6-(1H- 1,2,4-triazol-3-yl)-1H-imidazo[4,5-c]pyridin-1-yl)-1- methylpiperidin-3-yl)picolinamide |
| 133 | 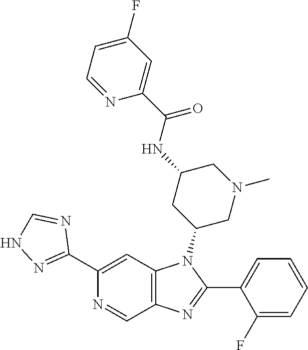 [SMILES: CN1C[C@@H](NC(=O)c2cc(F)ccn2)C[C@@H](n2c(-c3ccccc3F)nc3cnc(-c4nc[nH]n4)cc32)C1] | 4-fluoro-N-((3S,5R)-5-(2-(2-fluorophenyl)-6-(1H-1,2,4- triazol-3-yl)-1H-imidazo[4,5-c]pyridin-1-yl)-1- methylpiperidin-3-yl)picolinamide |
| 134 | 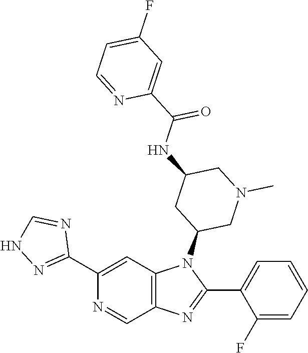 [SMILES: CN1C[C@H](NC(=O)c2cc(F)ccn2)C[C@H](n2c(-c3ccccc3F)nc3cnc(-c4nc[nH]n4)cc32)C1] | 4-fluoro-N-((3R,5S)-5-(2-(2-fluorophenyl)-6-(1H-1,2,4- triazol-3-yl)-1H-imidazo[4,5-c]pyridin-1-yl)-1- methylpiperidin-3-yl)picolinamide |
| 136 | 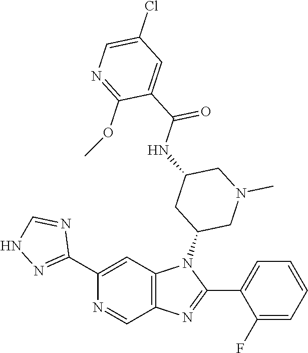 [SMILES: COc1ncc(Cl)cc1C(=O)N[C@H]1C[C@@H](n2c(-c3ccccc3F)nc3cnc(-c4nc[nH]n4)cc32)CN(C)C1] | 5-chloro-N-((3R,5S)-5-(2-(2-fluorophenyl)-6-(1H-1,2,4- triazol-3-yl)-1H-imidazo[4,5-c]pyridin-1-yl)-1- methylpiperidin-3-yl)-2-methoxybenzamide |
| 135 | 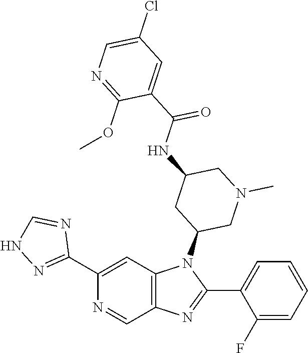 [SMILES: COc1ncc(Cl)cc1C(=O)N[C@@H]1C[C@H](n2c(-c3ccccc3F)nc3cnc(-c4nc[nH]n4)cc32)CN(C)C1] | 5-chloro-N-((3S,5R)-5-(2-(2-fluorophenyl)-6-(1H-1,2,4- triazol-3-yl)-1H-imidazo[4,5-c]pyridin-1-yl)-1- methylpiperidin-3-yl)-2-methoxybenzamide |
| 137 | 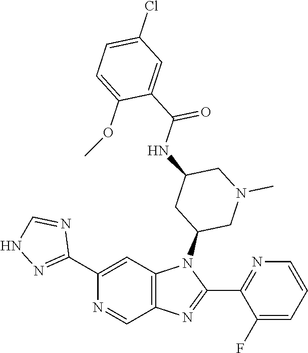 [SMILES: COc1ccc(Cl)cc1C(=O)N[C@@H]1C[C@H](n2c(-c3ncccc3F)nc3cnc(-c4nc[nH]n4)cc32)CN(C)C1] | 5-chloro-N-((3R,5S)-5-(2-(3-fluoropyridin-2-yl)-6-(1H- 1,2,4-triazol-3-yl)-1H-imidazo[4,5-c]pyridin-1-yl)-1- methylpiperidin-3-yl)-2-methoxybenzamide |
| 138 | 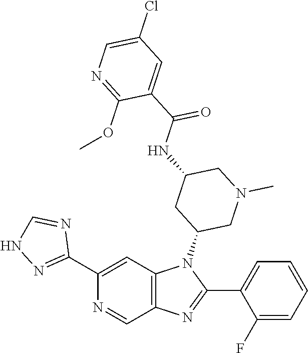 [SMILES: COc1ncc(Cl)cc1C(=O)N[C@H]1C[C@@H](n2c(-c3ccccc3F)nc3cnc(-c4nc[nH]n4)cc32)CN(C)C1] | 5-chloro-N-((3S,5R)-5-(2-(3-fluoropyridin-2-yl)-6-(1H- 1,2,4-triazol-3-yl)-1H-imidazo[4,5-c]pyridin-1-yl)-1- methylpiperidin-3-yl)-2-methoxybenzamide |
| 139 | 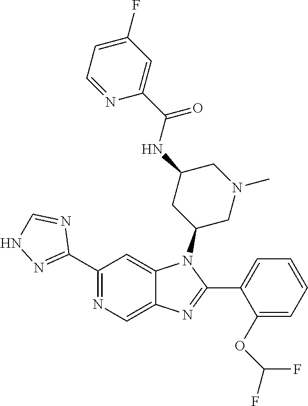 [SMILES: CN1C[C@H](NC(=O)c2cc(F)ccn2)C[C@H](n2c(-c3ccccc3OC(F)F)nc3cnc(-c4nc[nH]n4)cc32)C1] | N-((3R,5S)-5-(2-(2-(difluoromethoxy)phenyl)-6-(1H- 1,2,4-triazol-3-yl)-1H-imidazo[4,5-c]pyridin-1-yl)-1- methylpiperidin-3-yl)-4-fluoropicolinamide |
| 140 | 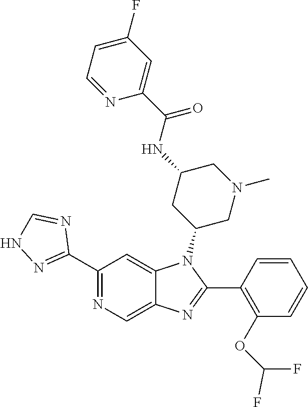 [SMILES: CN1C[C@@H](NC(=O)c2cc(F)ccn2)C[C@@H](n2c(-c3ccccc3OC(F)F)nc3cnc(-c4nc[nH]n4)cc32)C1] | N-((3S,5R)-5-(2-(2-(difluoromethoxy)phenyl)-6-(1H- 1,2,4-triazol-3-yl)-1H-imidazo[4,5-c]pyridin-1-yl)-1- methylpiperidin-3-yl)-4-fluoropicolinamide |
| 141 | 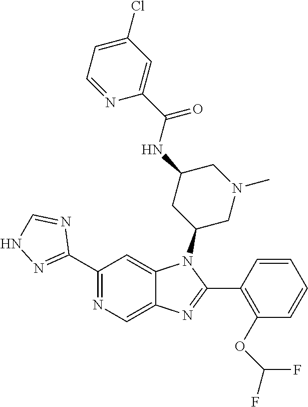 [SMILES: CN1C[C@H](NC(=O)c2cc(Cl)ccn2)C[C@H](n2c(-c3ccccc3OC(F)F)nc3cnc(-c4nc[nH]n4)cc32)C1] | 4-chloro-N-((3R,5S)-5-(2-(2-(difluoromethoxy)phenyl)- 6-(1H-1,2,4-triazol-3-yl)-1H-imidazo[4,5-c]pyridin-1-yl)- 1-methylpiperidin-3-yl)picolinamide |
| 142 | 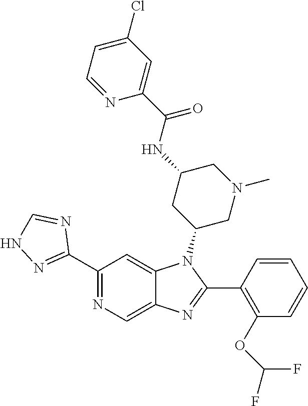 [SMILES: CN1C[C@@H](NC(=O)c2cc(Cl)ccn2)C[C@@H](n2c(-c3ccccc3OC(F)F)nc3cnc(-c4nc[nH]n4)cc32)C1] | 4-chloro-N-((3S,5R)-5-(2-(2-(difluoromethoxy)phenyl)- 6-(1H-1,2,4-triazol-3-yl)-1H-imidazo[4,5-c]pyridin-1-yl)- 1-methylpiperidin-3-yl)picolinamide |
| 143 | 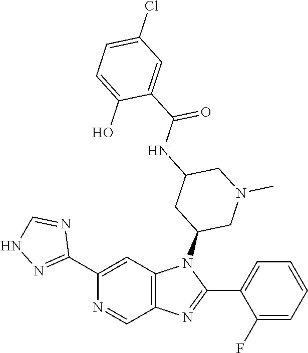 [SMILES: CN1CC(NC(=O)c2cc(Cl)ccc2O)C[C@H](n2c(-c3ccccc3F)nc3cnc(-c4nc[nH]n4)cc32)C1] | 5-chloro-N-(5-(2-(2-fluorophenyl)-6-(1H-1,2,4-triazol-3- yl)-1H-imidazo[4,5-c]pyridin-1-yl)-1-methylpiperidin-3- yl)-2-hydroxybenzamide⁽³⁾ ⁽³⁾ relative stereochemistry at centers 3 and 5 of piperidine ring is cis |
| 144 | 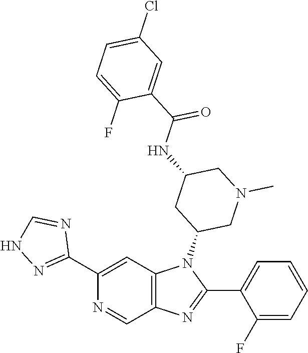 [SMILES: CN1C[C@@H](NC(=O)c2cc(Cl)ccc2F)C[C@@H](n2c(-c3ccccc3F)nc3cnc(-c4nc[nH]n4)cc32)C1] | 5-chloro-2-fluoro-N-((3S,5R)-5-(2-(2-fluorophenyl)-6- (1H-1,2,4-triazol-3-yl)-1H-imidazo[4,5-c]pyridin-1-yl)-1- methylpiperidin-3-yl)benzamide |
| 145 | 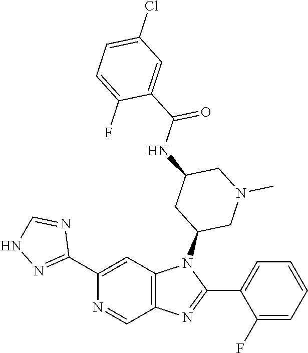 [SMILES: CN1C[C@H](NC(=O)c2cc(Cl)ccc2F)C[C@H](n2c(-c3ccccc3F)nc3cnc(-c4nc[nH]n4)cc32)C1] | 5-chloro-2-fluoro-N-((3R,5S)-5-(2-(2-fluorophenyl)-6- (1H-1,2,4-triazol-3-yl)-1H-imidazo[4,5-c]pyridin-1-yl)-1- methylpiperidin-3-yl)benzamide |
| 146 | 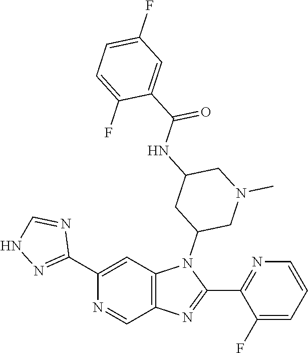 [SMILES: CN1CC(NC(=O)c2cc(F)ccc2F)CC(n2c(-c3ncccc3F)nc3cnc(-c4nc[nH]n4)cc32)C1] | 2,5-difluoro-N-(5-(2-(3-fluoropyridin-2-yl)-6-(1H-1,2,4- triazol-3-yl)-1H-imidazo[4,5-c]pyridin-1-yl)-1- methylpiperidin-3-yl)benzamide⁽³⁾ ⁽³⁾ relative stereochemistry at centers 3 and 5 of piperidine ring is cis |
| 147 | 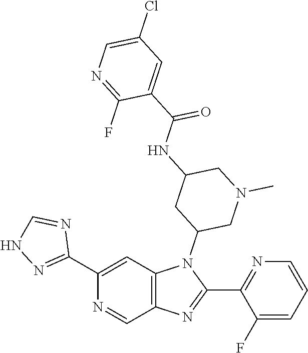 [SMILES: CN1CC(NC(=O)c2cc(Cl)cnc2F)CC(n2c(-c3ncccc3F)nc3cnc(-c4nc[nH]n4)cc32)C1] | 5-chloro-2-fluoro-N-(5-(2-(3-fluoropyridin-2-yl)-6-(1H- 1,2,4-triazol-3-yl)-1H-imidazo[4,5-c]pyridin-1-yl)-1- methylpiperidin-3-yl)benzamide⁽³⁾ ⁽³⁾ relative stereochemistry at centers 3 and 5 of piperidine ring is cis |
| 148 | 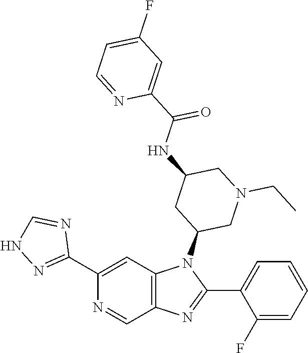 [SMILES: CCN1C[C@H](NC(=O)c2cc(F)ccn2)C[C@H](n2c(-c3ccccc3F)nc3cnc(-c4nc[nH]n4)cc32)C1] | N-((3R,5S)-1-ethyl-5-(2-(2-fluorophenyl)-6-(1H-1,2,4- triazol-3-yl)-1H-imidazo[4,5-c]pyridin-1-yl)piperidin-3- yl)-4-fluoropicolinamide |
| 149 | 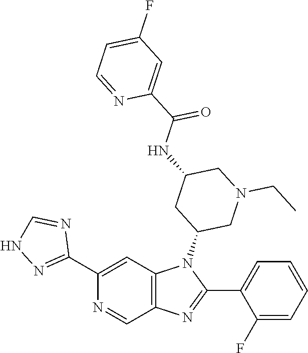 [SMILES: CCN1C[C@@H](NC(=O)c2cc(F)ccn2)C[C@@H](n2c(-c3ccccc3F)nc3cnc(-c4nc[nH]n4)cc32)C1] | N-((3S,5R)-1-ethyl-5-(2-(2-fluorophenyl)-6-(1H-1,2,4- triazol-3-yl)-1H-imidazo[4,5-c]pyridin-1-yl)piperidin-3- yl)-4-fluoropicolinamide |
| 150 | 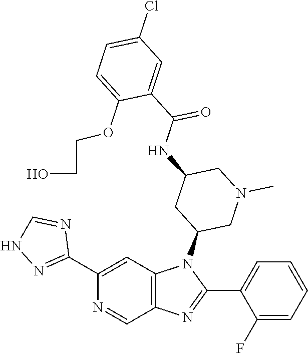 [SMILES: CN1C[C@H](NC(=O)c2cc(Cl)ccc2OCCO)C[C@H](n2c(-c3ccccc3F)nc3cnc(-c4nc[nH]n4)cc32)C1] | 5-chloro-N-((3R,5S)-5-(2-(2-fluorophenyl)-6-(1H-1,2,4- triazol-3-yl)-1H-imidazo[4,5-c]pyridin-1-yl)-1- methylpiperidin-3-yl)-2-(2-hydroxyethoxy)benzamide |
| 151 | 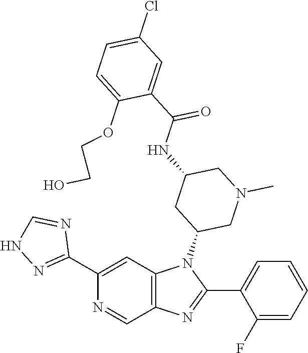 [SMILES: CN1C[C@@H](NC(=O)c2cc(Cl)ccc2OCCO)C[C@@H](n2c(-c3ccccc3F)nc3cnc(-c4nc[nH]n4)cc32)C1] | 5-chloro-N-((3S,5R)-5-(2-(2-fluorophenyl)-6-(1H-1,2,4- triazol-3-yl)-1H-imidazo[4,5-c]pyridin-1-yl)-1- methylpiperidin-3-yl)-2-(2-hydroxyethoxy)benzamide |
| 152 | 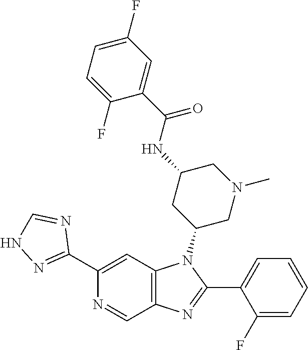 [SMILES: CN1C[C@@H](NC(=O)c2cc(F)ccc2F)C[C@@H](n2c(-c3ccccc3F)nc3cnc(-c4nc[nH]n4)cc32)C1] | 2,5-difluoro-N-((3S,5R)-5-(2-(2-fluorophenyl)-6-(1H- 1,2,4-triazol-3-yl)-1H-imidazo[4,5-c]pyridin-1-yl)-1- methylpiperidin-3-yl)benzamide |
| 153 | 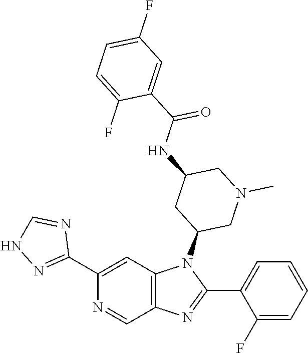 [SMILES: CN1C[C@H](NC(=O)c2cc(F)ccc2F)C[C@H](n2c(-c3ccccc3F)nc3cnc(-c4nc[nH]n4)cc32)C1] | 2,5-difluoro-N-((3R,5S)-5-(2-(2-fluorophenyl)-6-(1H- 1,2,4-triazol-3-yl)-1H-imidazo[4,5-c]pyridin-1-yl)-1- methylpiperidin-3-yl)benzamide |
| 154 | 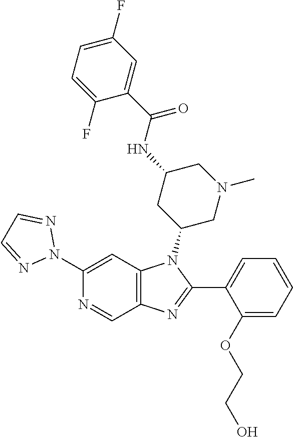 [SMILES: CN1C[C@@H](NC(=O)c2cc(F)ccc2F)C[C@@H](n2c(-c3ccccc3OCCO)nc3cnc(-n4nccn4)cc32)C1] | 2,5-difluoro-N-((3S,5R)-5-(2-(2-(2- hydroxyethoxy)phenyl)-6-(2H-1,2,3-triazol-2-yl)-1H- imidazo[4,5-c]pyridin-1-yl)-1-methylpiperidin-3- yl)benzamide |
| 155 | 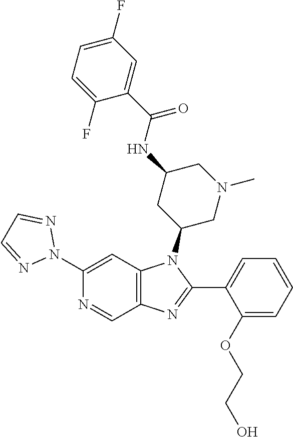 [SMILES: CN1C[C@H](NC(=O)c2cc(F)ccc2F)C[C@H](n2c(-c3ccccc3OCCO)nc3cnc(-n4nccn4)cc32)C1] | 2,5-difluoro-N-((3R,5S)-5-(2-(2-(2- hydroxyethoxy)phenyl)-6-(2H-1,2,3-triazol-2-yl)-1H- imidazo[4,5-c]pyridin-1-yl)-1-methylpiperidin-3- yl)benzamide |
| 156 | 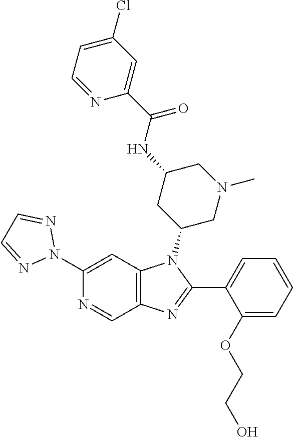 [SMILES: CN1C[C@@H](NC(=O)c2cc(Cl)ccn2)C[C@@H](n2c(-c3ccccc3OCCO)nc3cnc(-n4nccn4)cc32)C1] | 4-chloro-N-((3S,5R)-5-(2-(2-(2-hydroxyethoxy)phenyl)- 6-(2H-1,2,3-triazol-2-yl)-1H-imidazo[4,5-c]pyridin-1-yl)- 1-methylpiperidin-3-yl)picolinamide |
| 157 | 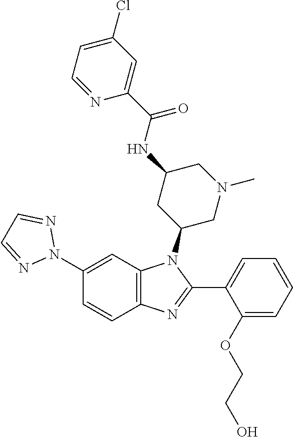 [SMILES: CN1C[C@H](NC(=O)c2cc(Cl)ccn2)C[C@H](n2c(-c3ccccc3OCCO)nc3ccc(-n4nccn4)cc32)C1] | 4-chloro-N-((3R,5S)-5-(2-(2-(2-hydroxyethoxy)phenyl)- 6-(2H-1,2,3-triazol-2-yl)-1H-imidazo[4,5-c]pyridin-1-yl)- 1-methylpiperidin-3-yl)picolinamide |
| 158 | 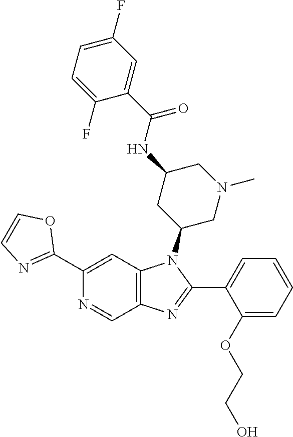 [SMILES: CN1C[C@H](NC(=O)c2cc(F)ccc2F)C[C@H](n2c(-c3ccccc3OCCO)nc3cnc(-c4ncco4)cc32)C1] | 2,5-difluoro-N-((3R,5S)-5-(2-(2-(2- hydroxyethoxy)phenyl)-6-(oxazol-2-yl)-1H-imidazo[4,5- c]pyridin-1-yl)-1-methylpiperidin-3-yl)benzamide |
| 159 | 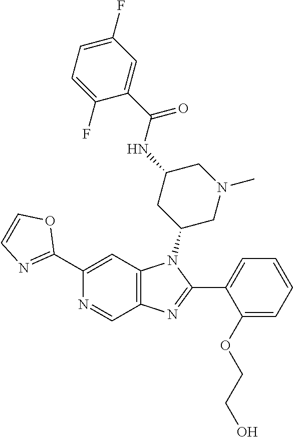 [SMILES: CN1C[C@@H](NC(=O)c2cc(F)ccc2F)C[C@@H](n2c(-c3ccccc3OCCO)nc3cnc(-c4ncco4)cc32)C1] | 2,5-difluoro-N-((3S,5R)-5-(2-(2-(2- hydroxyethoxy)phenyl)-6-(oxazol-2-yl)-1H-imidazo[4,5- c]pyridin-1-yl)-1-methylpiperidin-3-yl)benzamide |
| 160 | 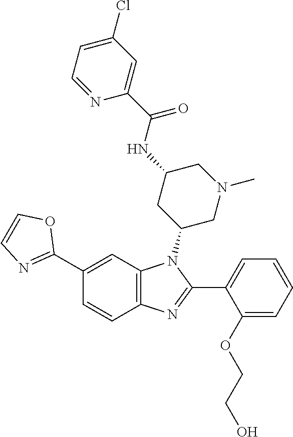 [SMILES: CN1C[C@@H](NC(=O)c2cc(Cl)ccn2)C[C@@H](n2c(-c3ccccc3OCCO)nc3ccc(-c4ncco4)cc32)C1] | 4-chloro-N-((3S,5R)-5-(2-(2-(2-hydroxyethoxy)phenyl)- 6-(oxazol-2-yl)-1H-imidazo[4,5-c]pyridin-1-yl)-1- methylpiperidin-3-yl)picolinamide |
| 161 | 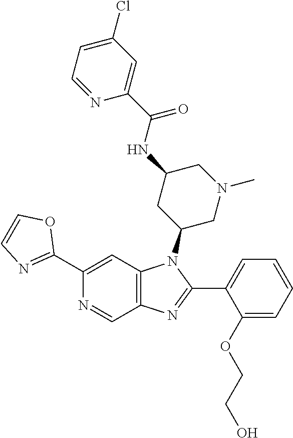 [SMILES: CN1C[C@H](NC(=O)c2cc(Cl)ccn2)C[C@H](n2c(-c3ccccc3OCCO)nc3cnc(-c4ncco4)cc32)C1] | 4-chloro-N-((3R,5S)-5-(2-(2-(2-hydroxyethoxy)phenyl)- 6-(oxazol-2-yl)-1H-imidazo[4,5-c]pyridin-1-yl)-1- methylpiperidin-3-yl)picolinamide |
| 162 | 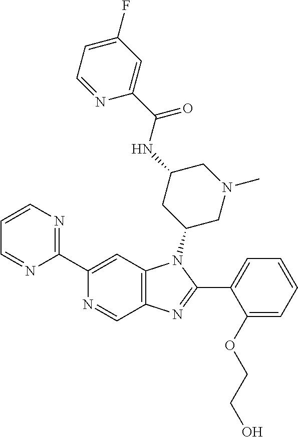 [SMILES: CN1C[C@@H](NC(=O)c2cc(F)ccn2)C[C@@H](n2c(-c3ccccc3OCCO)nc3cnc(-c4ncccn4)cc32)C1] | 4-fluoro-N-((3S,5R)-5-(2-(2-(2-hydroxyethoxy)phenyl)- 6-(pyrimidin-2-yl)-1H-imidazo[4,5-c]pyridin-1-yl)-1- methylpiperidin-3-yl)picolinamide |
| 163 | 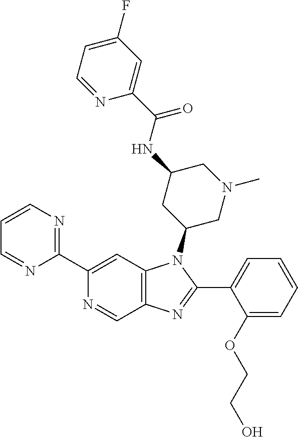 [SMILES: CN1C[C@H](NC(=O)c2cc(F)ccn2)C[C@H](n2c(-c3ccccc3OCCO)nc3cnc(-c4ncccn4)cc32)C1] | 4-fluoro-N-((3R,5S)-5-(2-(2-(2-hydroxyethoxy)phenyl)- 6-(pyrimidin-2-yl)-1H-imidazo[4,5-c]pyridin-1-yl)-1- methylpiperidin-3-yl)picolinamide |
| 164 | 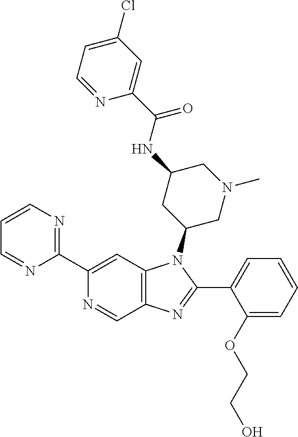 [SMILES: CN1C[C@H](NC(=O)c2cc(Cl)ccn2)C[C@H](n2c(-c3ccccc3OCCO)nc3cnc(-c4ncccn4)cc32)C1] | 4-chloro-N-((3R,5S)-5-(2-(2-(2-hydroxyethoxy)phenyl)- 6-(pyrimidin-2-yl)-1H-imidazo[4,5-c]pyridin-1-yl)-1- methylpiperidin-3-yl)picolinamide |
| 165 | 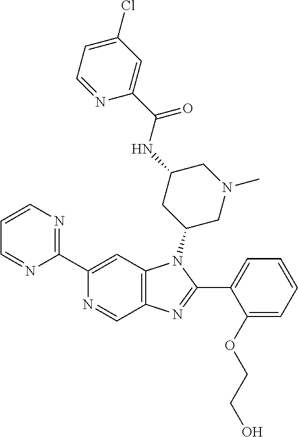 [SMILES: CN1C[C@@H](NC(=O)c2cc(Cl)ccn2)C[C@@H](n2c(-c3ccccc3OCCO)nc3cnc(-c4ncccn4)cc32)C1] | 4-chloro-N-((3S,5R)-5-(2-(2-(2-hydroxyethoxy)phenyl)- 6-(pyrimidin-2-yl)-1H-imidazo[4,5-c]pyridin-1-yl)-1- methylpiperidin-3-yl)picolinamide |
| 166 | 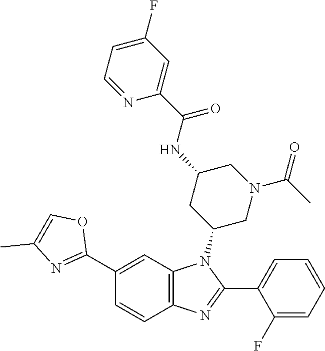 [SMILES: CC(=O)N1C[C@@H](NC(=O)c2cc(F)ccn2)C[C@@H](n2c(-c3ccccc3F)nc3ccc(-c4nc(C)co4)cc32)C1] | N-((3S,5R)-1-acetyl-5-(2-(2-fluorophenyl)-6-(4- methyloxazol-2-yl)-1H-imidazo[4,5-c]pyridin-1- yl)piperidin-3-yl)-4-fluoropicolinamide |
| 167 | 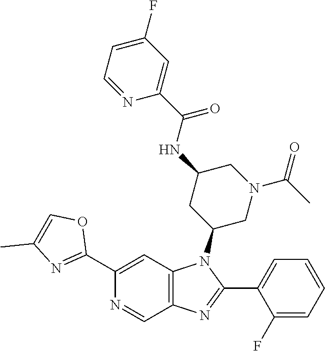 [SMILES: CC(=O)N1C[C@H](NC(=O)c2cc(F)ccn2)C[C@H](n2c(-c3ccccc3F)nc3cnc(-c4nc(C)co4)cc32)C1] | N-((3R,5S)-1-acetyl-5-(2-(2-fluorophenyl)-6-(4- methyloxazol-2-yl)-1H-imidazo[4,5-c]pyridin-1- yl)piperidin-3-yl)-4-fluoropicolinamide |
| 168 | 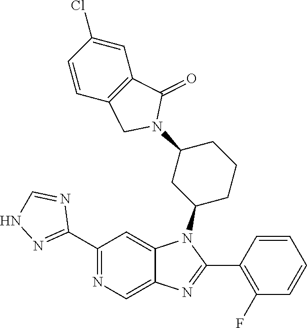 [SMILES: O=C1c2cc(Cl)ccc2CN1[C@H]1CCC[C@@H](n2c(-c3ccccc3F)nc3cnc(-c4nc[nH]n4)cc32)C1] | 6-chloro-2-((1S,3R)-3-(2-(2-fluorophenyl)-6-(1H-1,2,4- triazol-3-yl)-1H-imidazo[4,5-c]pyridin-1- yl)cyclohexyl)isoindolin-1-one |
| 169 | 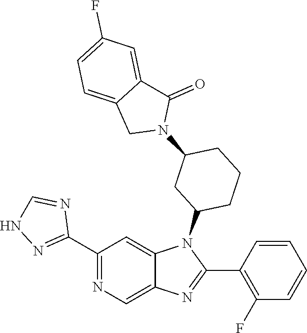 | 6-fluoro-2-((1S,3R)-3-(2-(2-fluorophenyl)-6-(1H-1,2,4- triazol-3-yl)-1H-imidazo[4,5-c]pyridin-1- yl)cyclohexyl)isoindolin-1-one |
| 170 | 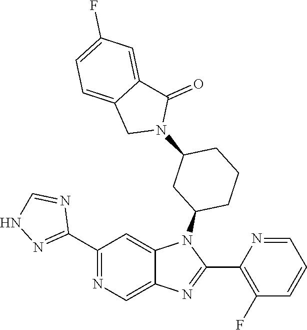 [SMILES: O=C1c2cc(F)ccc2CN1[C@H]1CCC[C@@H](n2c(-c3ncccc3F)nc3cnc(-c4nc[nH]n4)cc32)C1] | 6-fluoro-2-((1S,3R)-3-(2-(3-fluoropyridin-2-yl)-6-(1H- 1,2,4-triazol-3-yl)-1H-imidazo[4,5-c]pyridin-1- yl)cyclohexyl)isoindolin-1-one |
| 171 | 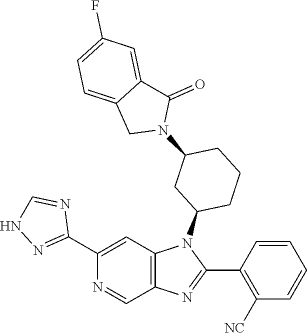 [SMILES: N#Cc1ccccc1-c1nc2cnc(-c3nc[nH]n3)cc2n1[C@@H]1CCC[C@H](N2Cc3ccc(F)cc3C2=O)C1] | 2-(1-((1R,3S)-3-(6-fluoro-1-oxoisoindolin-2- yl)cyclohexyl)-6-(1H-1,2,4-triazol-3-yl)-1H-imidazo[4,5- c]pyridin-2-yl)benzonitrile |
| 172 | 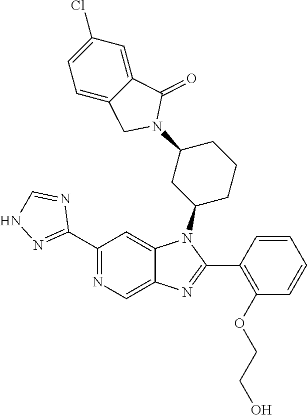 [SMILES: O=C1c2cc(Cl)ccc2CN1[C@H]1CCC[C@@H](n2c(-c3ccccc3OCCO)nc3cnc(-c4nc[nH]n4)cc32)C1] | 6-chloro-2-((1S,3R)-3-(2-(2-(2-hydroxyethoxy)phenyl)- 6-(1H-1,2,4-triazol-3-yl)-1H-imidazo[4,5-c]pyridin-1- yl)cyclohexyl)isoindolin-1-one |
| 173 | 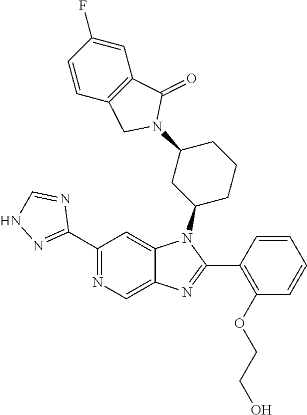 [SMILES: O=C1c2cc(F)ccc2CN1[C@H]1CCC[C@@H](n2c(-c3ccccc3OCCO)nc3cnc(-c4nc[nH]n4)cc32)C1] | 6-fluoro-2-((1S,3R)-3-(2-(2-(2-hydroxyethoxy)phenyl)-6- (1H-1,2,4-triazol-3-yl)-1H-imidazo[4,5-c]pyridin-1- yl)cyclohexyl)isoindolin-1-one |
| 174 | 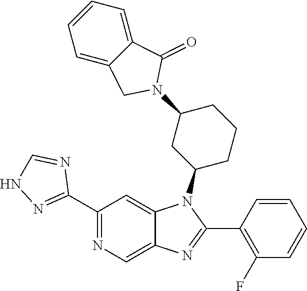 [SMILES: O=C1c2ccccc2CN1[C@H]1CCC[C@@H](n2c(-c3ccccc3F)nc3cnc(-c4nc[nH]n4)cc32)C1] | 2-((1S,3R)-3-(2-(2-fluorophenyl)-6-(1H-1,2,4-triazol-3- yl)-1H-imidazo[4,5-c]pyridin-1-yl)cyclohexyl)isoindolin- 1-one |
| 175 | 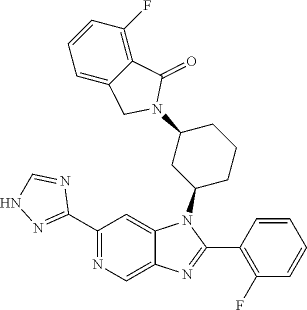 [SMILES: O=C1c2c(F)cccc2CN1[C@H]1CCC[C@@H](n2c(-c3ccccc3F)nc3cnc(-c4nc[nH]n4)cc32)C1] | 7-fluoro-2-((1S,3R)-3-(2-(2-fluorophenyl)-6-(1H-1,2,4- triazol-3-yl)-1H-imidazo[4,5-c]pyridin-1- yl)cyclohexyl)isoindolin-1-one |
| 176 | 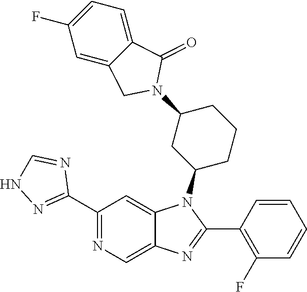 [SMILES: O=C1c2ccc(F)cc2CN1[C@H]1CCC[C@@H](n2c(-c3ccccc3F)nc3cnc(-c4nc[nH]n4)cc32)C1] | 5-fluoro-2-((1S,3R)-3-(2-(2-fluorophenyl)-6-(1H-1,2,4- triazol-3-yl)-1H-imidazo[4,5-c]pyridin-1- yl)cyclohexyl)isoindolin-1-one |
| 177 | 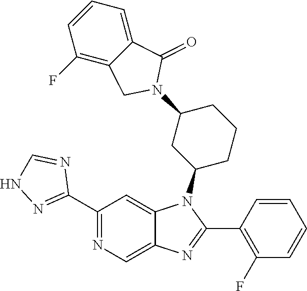 [SMILES: O=C1c2cccc(F)c2CN1[C@H]1CCC[C@@H](n2c(-c3ccccc3F)nc3cnc(-c4nc[nH]n4)cc32)C1] | 4-fluoro-2-((1S,3R)-3-(2-(2-fluorophenyl)-6-(1H-1,2,4- triazol-3-yl)-1H-imidazo[4,5-c]pyridin-1- yl)cyclohexyl)isoindolin-1-one |
| 178 | 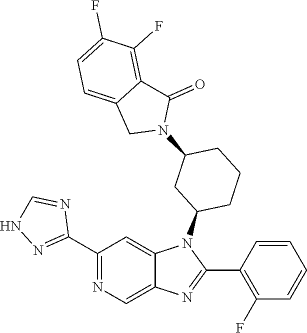 [SMILES: O=C1c2c(ccc(F)c2F)CN1[C@H]1CCC[C@@H](n2c(-c3ccccc3F)nc3cnc(-c4nc[nH]n4)cc32)C1] | 6,7-difluoro-2-((1S,3R)-3-(2-(2-fluorophenyl)-6-(1H- 1,2,4-triazol-3-yl)-1H-imidazo[4,5-c]pyridin-1- yl)cyclohexyl)isoindolin-1-one |
| 179 | 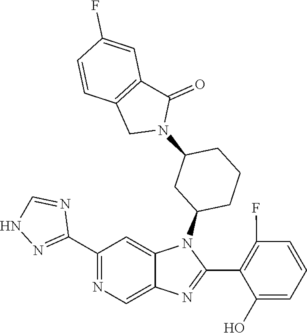 [SMILES: O=C1c2cc(F)ccc2CN1[C@H]1CCC[C@@H](n2c(-c3c(O)cccc3F)nc3cnc(-c4nc[nH]n4)cc32)C1] | 6-fluoro-2-((1S,3R)-3-(2-(2-fluoro-6-hydroxyphenyl)-6- (1H-1,2,4-triazol-3-yl)-1H-imidazo[4,5-c]pyridin-1- yl)cyclohexyl)isoindolin-1-one |
| 180 | 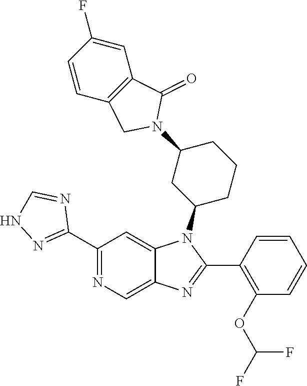 [SMILES: O=C1c2cc(F)ccc2CN1[C@H]1CCC[C@@H](n2c(-c3ccccc3OC(F)F)nc3cnc(-c4nc[nH]n4)cc32)C1] | 2-((1S,3R)-3-(2-(2-(difluoromethoxy)phenyl)-6-(1H- 1,2,4-triazol-3-yl)-1H-imidazo[4,5-c]pyridin-1- yl)cyclohexyl)-6-fluoroisoindolin-1-one |
| 181 | 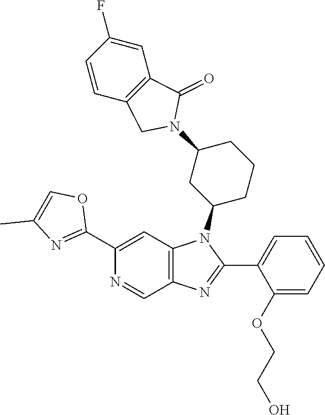 [SMILES: Cc1coc(-c2cc3c(cn2)nc(-c2ccccc2OCCO)n3[C@@H]2CCC[C@H](N3Cc4ccc(F)cc4C3=O)C2)n1] | 6-fluoro-2-((1S,3R)-3-(2-(2-(2-hydroxyethoxy)phenyl)-6- (4-methyloxazol-2-yl)-1H-imidazo[4,5-c]pyridin-1- yl)cyclohexyl)isoindolin-1-one |
| 182 | 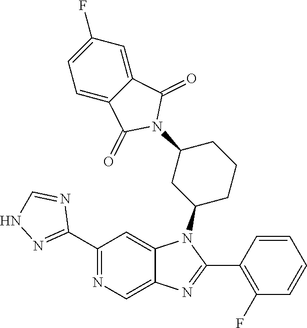 [SMILES: O=C1c2ccc(F)cc2C(=O)N1[C@H]1CCC[C@@H](n2c(-c3ccccc3F)nc3cnc(-c4nc[nH]n4)cc32)C1] | 5-fluoro-2-((1S,3R)-3-(2-(2-fluorophenyl)-6-(1H-1,2,4- triazol-3-yl)-1H-imidazo[4,5-c]pyridin-1- yl)cyclohexyl)isoindoline-1,3-dione |
| 183 | 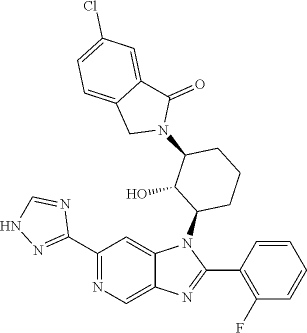 [SMILES: O=C1c2cc(Cl)ccc2CN1[C@H]1CCC[C@@H](n2c(-c3ccccc3F)nc3cnc(-c4nc[nH]n4)cc32)[C@@H]1O] | 6-chloro-2-((1S,2S,3R)-3-(2-(2-fluorophenyl)-6-(1H- 1,2,4-triazol-3-yl)-1H-imidazo[4,5-c]pyridin-1-yl)-2- hydroxycyclohexyl)isoindolin-1-one |
| 184 | 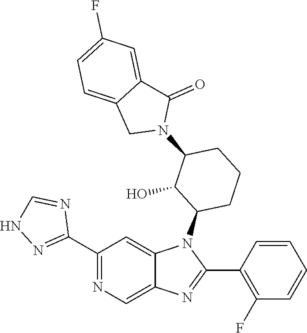 [SMILES: O=C1c2cc(F)ccc2CN1[C@H]1CCC[C@@H](n2c(-c3ccccc3F)nc3cnc(-c4nc[nH]n4)cc32)[C@@H]1O] | 6-fluoro-2-((1S,2S,3R)-3-(2-(2-fluorophenyl)-6-(1H- 1,2,4-triazol-3-yl)-1H-imidazo[4,5-c]pyridin-1-yl)-2- hydroxycyclohexyl)isoindolin-1-one |
| 185 | 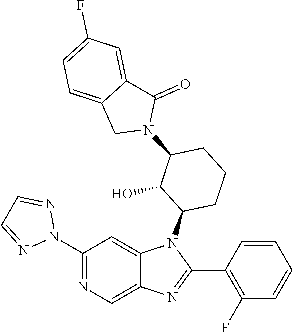 [SMILES: O=C1c2cc(F)ccc2CN1[C@H]1CCC[C@@H](n2c(-c3ccccc3F)nc3cnc(-n4nccn4)cc32)[C@@H]1O] | 6-fluoro-2-((1S,2S,3R)-3-(2-(2-fluorophenyl)-6-(2H- 1,2,3-triazol-2-yl)-1H-imidazo[4,5-c]pyridin-1-yl)-2- hydroxycyclohexyl)isoindolin-1-one |
| 186 | 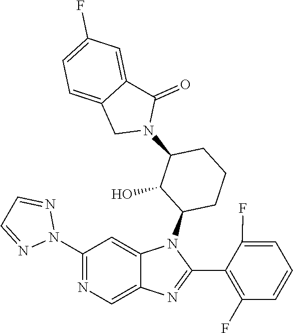 [SMILES: O=C1c2cc(F)ccc2CN1[C@H]1CCC[C@@H](n2c(-c3c(F)cccc3F)nc3cnc(-n4nccn4)cc32)[C@@H]1O] | 2-((1S,2S,3R)-3-(2-(2,6-difluorophenyl)-6-(2H-1,2,3- triazol-2-yl)-1H-imidazo[4,5-c]pyridin-1-yl)-2- hydroxycyclohexyl)-6-fluoroisoindolin-1-one |
| 187 | 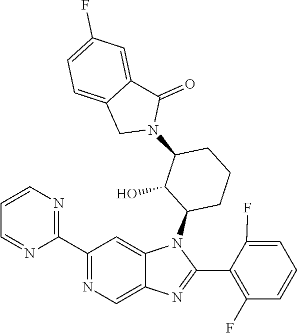 [SMILES: O=C1c2cc(F)ccc2CN1[C@H]1CCC[C@@H](n2c(-c3c(F)cccc3F)nc3cnc(-c4ncccn4)cc32)[C@@H]1O] | 2-((1S,2S,3R)-3-(2-(2,6-difluorophenyl)-6-(pyrimidin-2- yl)-1H-imidazo[4,5-c]pyridin-1-yl)-2- hydroxycyclohexyl)-6-fluoroisoindolin-1-one |
| 188 | 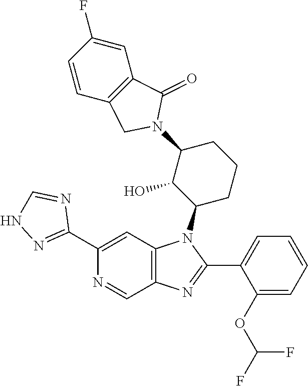 [SMILES: O=C1c2cc(F)ccc2CN1[C@H]1CCC[C@@H](n2c(-c3ccccc3OC(F)F)nc3cnc(-c4nc[nH]n4)cc32)[C@@H]1O] | 2-((1S,2S,3R)-3-(2-(2-(difluoromethoxy)phenyl)-6-(1H- 1,2,4-triazol-3-yl)-1H-imidazo[4,5-c]pyridin-1-yl)-2- hydroxycyclohexyl)-6-fluoroisoindolin-1-one |
| 189 | 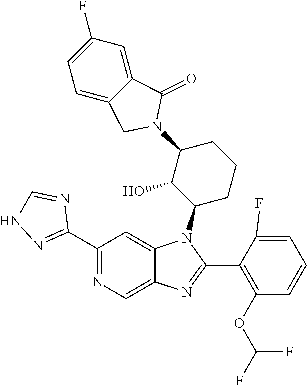 [SMILES: O=C1c2cc(F)ccc2CN1[C@H]1CCC[C@@H](n2c(-c3c(F)cccc3OC(F)F)nc3cnc(-c4nc[nH]n4)cc32)[C@@H]1O] | 2-((1S,2S,3R)-3-(2-(2-(difluoromethoxy)-6- fluorophenyl)-6-(1H-1,2,4-triazol-3-yl)-1H-imidazo[4,5- c]pyridin-1-yl)-2-hydroxycyclohexyl)-6-fluoroisoindolin- 1-one |
| 190 | 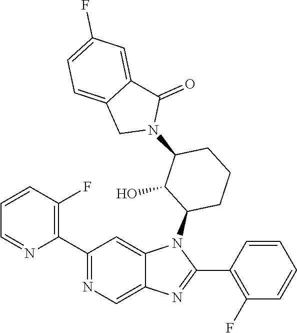 [SMILES: O=C1c2cc(F)ccc2CN1[C@H]1CCC[C@@H](n2c(-c3ccccc3F)nc3cnc(-c4ncccc4F)cc32)[C@@H]1O] | 6-fluoro-2-((1S,2S,3R)-3-(2-(2-fluorophenyl)-6-(3- fluoropyridin-2-yl)-1H-imidazo[4,5-c]pyridin-1-yl)-2- hydroxycyclohexyl)isoindolin-1-one |
| 191 | 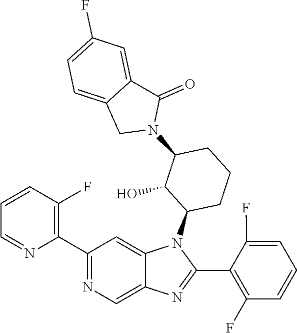 [SMILES: O=C1c2cc(F)ccc2CN1[C@H]1CCC[C@@H](n2c(-c3c(F)cccc3F)nc3cnc(-c4ncccc4F)cc32)[C@@H]1O] | 2-((1S,2S,3R)-3-(2-(2,6-difluorophenyl)-6-(3- fluoropyridin-2-yl)-1H-imidazo[4,5-c]pyridin-1-yl)-2- hydroxycyclohexyl)-6-fluoroisoindolin-1-one |
| 192 | 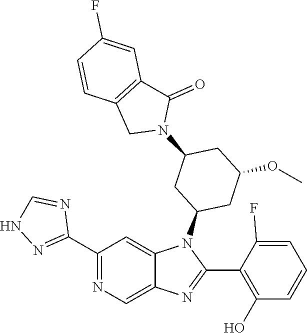 [SMILES: CO[C@H]1C[C@H](N2Cc3ccc(F)cc3C2=O)C[C@H](n2c(-c3c(O)cccc3F)nc3cnc(-c4nc[nH]n4)cc32)C1] | 6-fluoro-2-((1R,3S,5R)-3-(2-(2-fluoro-6-hydroxyphenyl)- 6-(1H-1,2,4-triazol-3-yl)-1H-imidazo[4,5-c]pyridin-1-yl)- 5-methoxycyclohexyl)isoindolin-1-one |
| 193 | 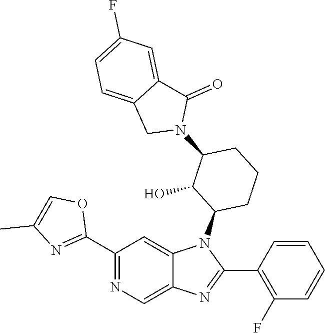 [SMILES: Cc1coc(-c2cc3c(cn2)nc(-c2ccccc2F)n3[C@@H]2CCC[C@H](N3Cc4ccc(F)cc4C3=O)[C@H]2O)n1] | 6-fluoro-2-((1S,2S,3R)-3-(2-(2-fluorophenyl)-6-(4- methyloxazol-2-yl)-1H-imidazo[4,5-c]pyridin-1-yl)-2- hydroxycyclohexyl)isoindolin-1-one |
| 194 | 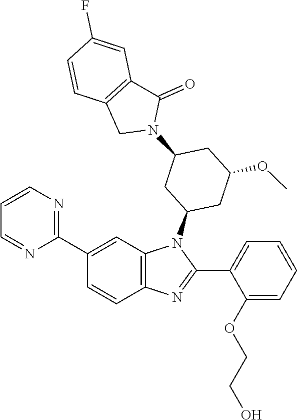 [SMILES: CO[C@H]1C[C@H](N2Cc3ccc(F)cc3C2=O)C[C@H](n2c(-c3ccccc3OCCO)nc3ccc(-c4ncccn4)cc32)C1] | 6-fluoro-2-((1R,3S,5R)-3-(2-(2-(2- hydroxyethoxy)phenyl)-6-(pyrimidin-2-yl)-1H- benzo[d]imidazol-1-yl)-5-methoxycyclohexyl)isoindolin- 1-one |
| 195 | 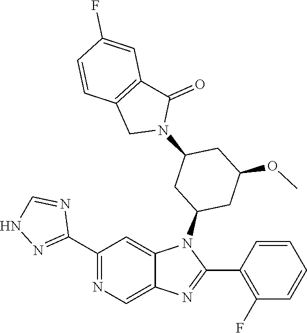 [SMILES: CO[C@@H]1C[C@H](N2Cc3ccc(F)cc3C2=O)C[C@H](n2c(-c3ccccc3F)nc3cnc(-c4nc[nH]n4)cc32)C1] | 6-fluoro-2-((1R,3S,5S)-3-(2-(2-fluorophenyl)-6-(1H- 1,2,4-triazol-3-yl)-1H-imidazo[4,5-c]pyridin-1-yl)-5- methoxycyclohexyl)isoindolin-1-one |
| 196 | 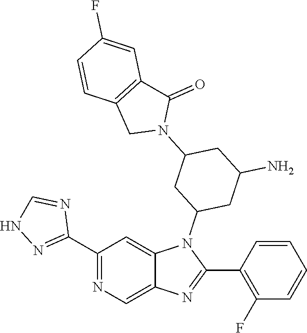 [SMILES: NC1CC(N2Cc3ccc(F)cc3C2=O)CC(n2c(-c3ccccc3F)nc3cnc(-c4nc[nH]n4)cc32)C1] | 2-(3-amino-5-(2-(2-fluorophenyl)-6-(1H-1,2,4-triazol-3- yl)-1H-imidazo[4,5-c]pyridin-1-yl)cyclohexyl)-6- fluoroisoindolin-1-one⁽⁴⁾ ⁽⁴⁾ relative stereochemistry at centers 1 and 5 of the cyclohexyl ring is cis with —NH₂ at center 3 cis |
| 197 | 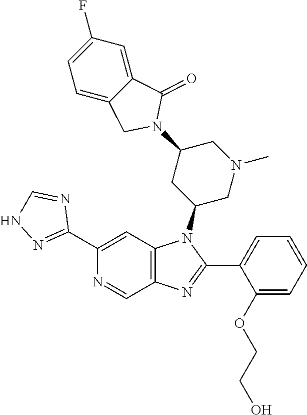 [SMILES: CN1C[C@H](N2Cc3ccc(F)cc3C2=O)C[C@H](n2c(-c3ccccc3OCCO)nc3cnc(-c4nc[nH]n4)cc32)C1] | 6-fluoro-2-((3R,5S)-5-(2-(2-(2-hydroxyethoxy)phenyl)-6- (1H-1,2,4-triazol-3-yl)-1H-imidazo[4,5-c]pyridin-1-yl)-1- methylpiperidin-3-yl)isoindolin-1-one |
| 198 | 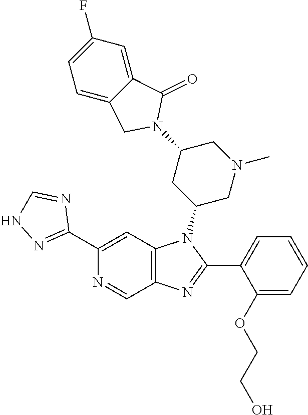 [SMILES: CN1C[C@@H](N2Cc3ccc(F)cc3C2=O)C[C@@H](n2c(-c3ccccc3OCCO)nc3cnc(-c4nc[nH]n4)cc32)C1] | 6-fluoro-2-((3S,5R)-5-(2-(2-(2-hydroxyethoxy)phenyl)-6- (1H-1,2,4-triazol-3-yl)-1H-imidazo[4,5-c]pyridin-1-yl)-1- methylpiperidin-3-yl)isoindolin-1-one |
| 199 | 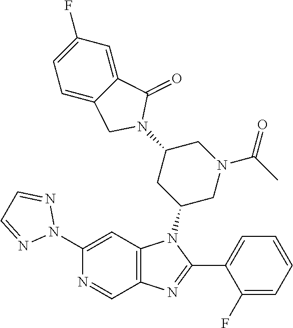 [SMILES: CC(=O)N1C[C@@H](N2Cc3ccc(F)cc3C2=O)C[C@@H](n2c(-c3ccccc3F)nc3cnc(-n4nccn4)cc32)C1] | 2-((3S,5R)-1-acetyl-5-(2-(2-fluorophenyl)-6-(2H-1,2,3- triazol-2-yl)-1H-imidazo[4,5-c]pyridin-1-yl)piperidin-3- yl)-6-fluoroisoindolin-1-one |
| 200 | 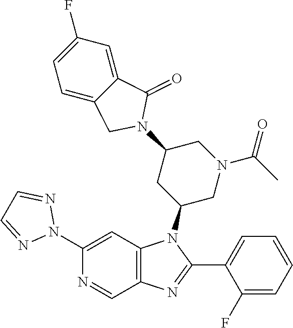 [SMILES: CC(=O)N1C[C@H](N2Cc3ccc(F)cc3C2=O)C[C@H](n2c(-c3ccccc3F)nc3cnc(-n4nccn4)cc32)C1] | 2-((3R,5S)-1-acetyl-5-(2-(2-fluorophenyl)-6-(2H-1,2,3- triazol-2-yl)-1H-imidazo[4,5-c]pyridin-1-yl)piperidin-3- yl)-6-fluoroisoindolin-1-one |
| 201 | 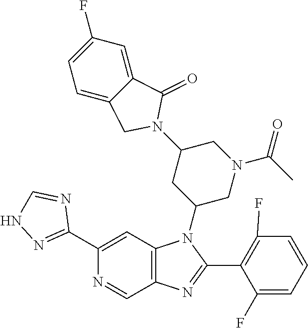 [SMILES: CC(=O)N1CC(N2Cc3ccc(F)cc3C2=O)CC(n2c(-c3c(F)cccc3F)nc3cnc(-c4nc[nH]n4)cc32)C1] | 2-(1-acetyl-5-(2-(2,6-difluorophenyl)-6-(1H-1,2,4-triazol- 3-yl)-1H-imidazo[4,5-c]pyridin-1-yl)piperidin-3-yl)-6- fluoroisoindolin-1-one⁽³⁾ ⁽³⁾ relative stereochemistry at centers 3 and 5 of piperidine ring is cis |
| 202 | 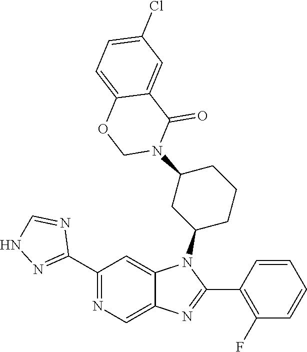 [SMILES: O=C1c2cc(Cl)ccc2OCN1[C@H]1CCC[C@@H](n2c(-c3ccccc3F)nc3cnc(-c4nc[nH]n4)cc32)C1] | 6-chloro-3-((1S,3R)-3-(2-(2-fluorophenyl)-6-(1H-1,2,4- triazol-3-yl)-1H-imidazo[4,5-c]pyridin-1-yl)cyclohexyl)- 2,3-dihydro-4H-benzo[e][1,3]oxazin-4-one |
| 203 | 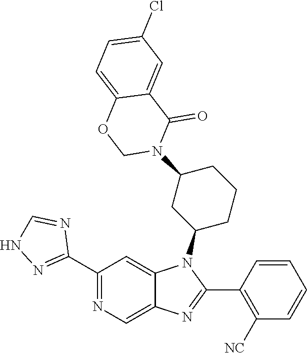 [SMILES: N#Cc1ccccc1-c1nc2cnc(-c3nc[nH]n3)cc2n1[C@@H]1CCC[C@H](N2COc3ccc(Cl)cc3C2=O)C1] | 2-(1-((1R,3S)-3-(6-chloro-4-oxo-2H- benzo[e][1,3]oxazin-3(4H)-yl)cyclohexyl)-6-(1H-1,2,4- triazol-3-yl)-1H-imidazo[4,5-c]pyridin-2-yl)benzonitrile |
| 204 | 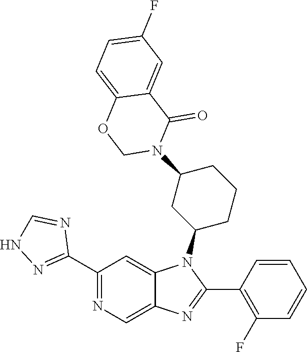 [SMILES: O=C1c2cc(F)ccc2OCN1[C@H]1CCC[C@@H](n2c(-c3ccccc3F)nc3cnc(-c4nc[nH]n4)cc32)C1] | 6-fluoro-3-((1S,3R)-3-(2-(2-fluorophenyl)-6-(1H-1,2,4- triazol-3-yl)-1H-imidazo[4,5-c]pyridin-1-yl)cyclohexyl)- 2,3-dihydro-4H-benzo[e][1,3]oxazin-4-one |
| 205 | 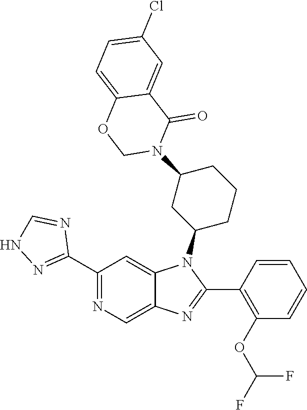 [SMILES: O=C1c2cc(Cl)ccc2OCN1[C@H]1CCC[C@@H](n2c(-c3ccccc3OC(F)F)nc3cnc(-c4nc[nH]n4)cc32)C1] | 6-chloro-3-((1S,3R)-3-(2-(2-(difluoromethoxy)phenyl)-6- (1H-1,2,4-triazol-3-yl)-1H-imidazo[4,5-c]pyridin-1- yl)cyclohexyl)-2,3-dihydro-4H-benzo[e][1,3]oxazin-4- one |
| 206 | 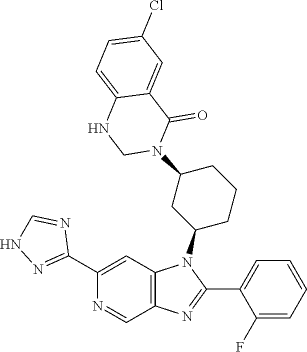 [SMILES: O=C1c2cc(Cl)ccc2NCN1[C@H]1CCC[C@@H](n2c(-c3ccccc3F)nc3cnc(-c4nc[nH]n4)cc32)C1] | 6-chloro-3-((1S,3R)-3-(2-(2-fluorophenyl)-6-(1H-1,2,4- triazol-3-yl)-1H-imidazo[4,5-c]pyridin-1-yl)cyclohexyl)- 2,3-dihydroquinazolin-4(1H)-one |
| 207 | 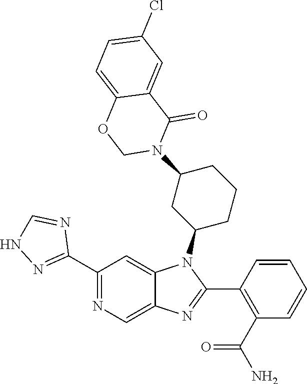 [SMILES: NC(=O)c1ccccc1-c1nc2cnc(-c3nc[nH]n3)cc2n1[C@@H]1CCC[C@H](N2COc3ccc(Cl)cc3C2=O)C1] | 2-(1-((1R,3S)-3-(6-chloro-4-oxo-2H- benzo[e][1,3]oxazin-3(4H)-yl)cyclohexyl)-6-(1H-1,2,4- triazol-3-yl)-1H-imidazo[4,5-c]pyridin-2-yl)benzamide |
| 208 | 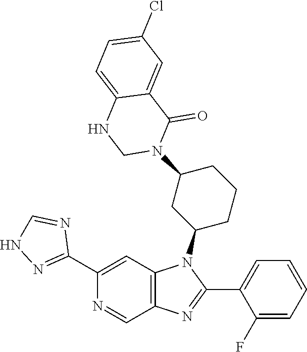 [SMILES: O=C1c2cc(Cl)ccc2NCN1[C@H]1CCC[C@@H](n2c(-c3ccccc3F)nc3cnc(-c4nc[nH]n4)cc32)C1] | 6-fluoro-3-((1S,3R)-3-(2-(2-fluorophenyl)-6-(1H-1,2,4- triazol-3-yl)-1H-imidazo[4,5-c]pyridin-1-yl)cyclohexyl)- 2,3-dihydroquinazolin-4(1H)-one |
| 209 | 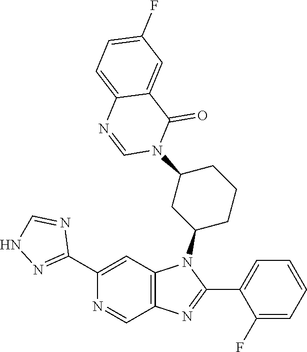 [SMILES: O=c1c2cc(F)ccc2ncn1[C@H]1CCC[C@@H](n2c(-c3ccccc3F)nc3cnc(-c4nc[nH]n4)cc32)C1] | 6-fluoro-3-((1S,3R)-3-(2-(2-fluorophenyl)-6-(1H-1,2,4- triazol-3-yl)-1H-imidazo[4,5-c]pyridin-1- yl)cyclohexyl)quinazolin-4(3H)-one |
| 210 | 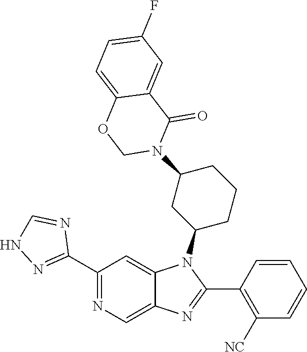 [SMILES: N#Cc1ccccc1-c1nc2cnc(-c3nc[nH]n3)cc2n1[C@@H]1CCC[C@H](N2COc3ccc(F)cc3C2=O)C1] | 2-(1-((1R,3S)-3-(6-fluoro-4-oxo-2H-benzo[e][1,3]oxazin- 3(4H)-yl)cyclohexyl)-6-(1H-1,2,4-triazol-3-yl)-1H- imidazo[4,5-c]pyridin-2-yl)benzonitrile |
| 211 | 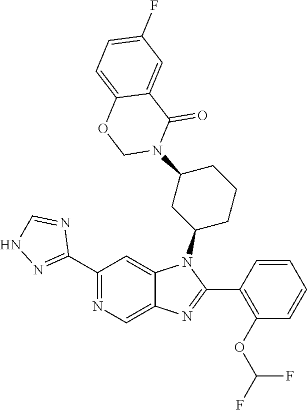 [SMILES: O=C1c2cc(F)ccc2OCN1[C@H]1CCC[C@@H](n2c(-c3ccccc3OC(F)F)nc3cnc(-c4nc[nH]n4)cc32)C1] | 3-((1S,3R)-3-(2-(2-(difluoromethoxy)phenyl)-6-(1H- 1,2,4-triazol-3-yl)-1H-imidazo[4,5-c]pyridin-1- yl)cyclohexyl)-6-fluoro-2,3-dihydro-4H- benzo[e][1,3]oxazin-4-one |
| 212 | 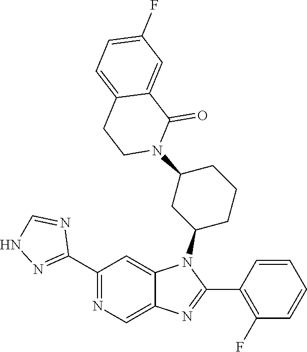 [SMILES: O=C1c2cc(F)ccc2CCN1[C@H]1CCC[C@@H](n2c(-c3ccccc3F)nc3cnc(-c4nc[nH]n4)cc32)C1] | 7-fluoro-2-((1S,3R)-3-(2-(2-fluorophenyl)-6-(1H-1,2,4- triazol-3-yl)-1H-imidazo[4,5-c]pyridin-1-yl)cyclohexyl)- 3,4-dihydroisoquinolin-1(2H)-one |
| 213 | 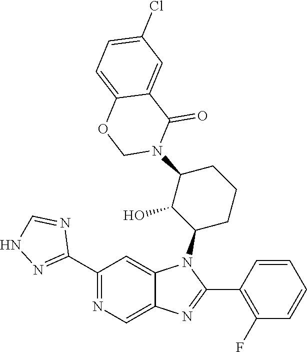 [SMILES: O=C1c2cc(Cl)ccc2OCN1[C@H]1CCC[C@@H](n2c(-c3ccccc3F)nc3cnc(-c4nc[nH]n4)cc32)[C@@H]1O] | 6-chloro-3-((1S,2S,3R)-3-(2-(2-fluorophenyl)-6-(1H- 1,2,4-triazol-3-yl)-1H-imidazo[4,5-c]pyridin-1-yl)-2- hydroxycyclohexyl)-2,3-dihydro-4H- benzo[e][1,3]oxazin-4-one |
| 214 | 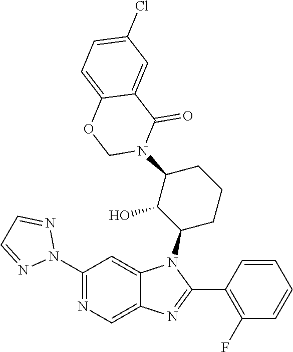 [SMILES: O=C1c2cc(Cl)ccc2OCN1[C@H]1CCC[C@@H](n2c(-c3ccccc3F)nc3cnc(-n4nccn4)cc32)[C@@H]1O] | 6-chloro-3-((1S,2S,3R)-3-(2-(2-fluorophenyl)-6-(2H- 1,2,3-triazol-2-yl)-1H-imidazo[4,5-c]pyridin-1-yl)-2- hydroxycyclohexyl)-2,3-dihydro-4H- benzo[e][1,3]oxazin-4-one |
| 215 | 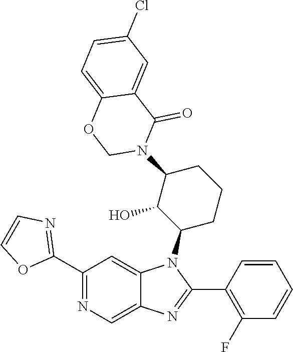 [SMILES: O=C1c2cc(Cl)ccc2OCN1[C@H]1CCC[C@@H](n2c(-c3ccccc3F)nc3cnc(-c4ncco4)cc32)[C@@H]1O] | 6-chloro-3-((1S,2S,3R)-3-(2-(2-fluorophenyl)-6-(oxazol- 2-yl)-1H-imidazo[4,5-c]pyridin-1-yl)-2- hydroxycyclohexyl)-2,3-dihydro-4H- benzo[e][1,3]oxazin-4-one |
| 216 | 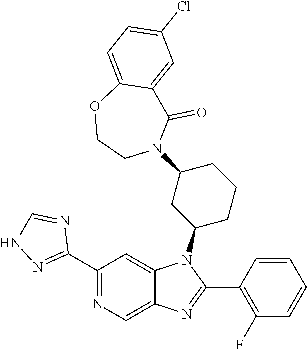 [SMILES: O=C1c2cc(Cl)ccc2OCCN1[C@H]1CCC[C@@H](n2c(-c3ccccc3F)nc3cnc(-c4nc[nH]n4)cc32)C1] | 7-chloro-4-((1S,3R)-3-(2-(2-fluorophenyl)-6-(1H-1,2,4- triazol-3-yl)-1H-imidazo[4,5-c]pyridin-1-yl)cyclohexyl)- 3,4-dihydrobenzo[f][1,4]oxazepin-5(2H)-one |

In some embodiments, the disclosure provides additional prophetic examples of Formula I, and their pharmaceutically acceptable salts and/or isotopologues, as set forth in Table 2 below. The prophetic compounds in Table 2 may be made in accordance with the procedures described in the General Schemes, Intermediate Schemes, and Examples herein alone or in combination with knowledge of a person of ordinary skill in the art.

TABLE 2

| Ex. | | |
|---|---|---|
| No. | Structure | Name |
| 300 | 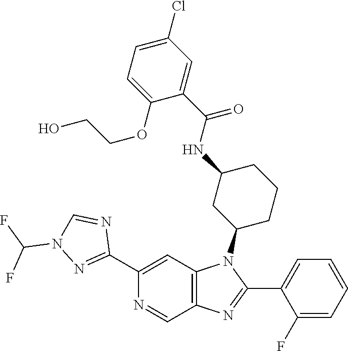 [SMILES: O=C(N[C@H]1CCC[C@@H](n2c(-c3ccccc3F)nc3cnc(-c4ncn(C(F)F)n4)cc32)C1)c1cc(Cl)ccc1OCCO] | 5-chloro-N-((1S,3R)-3-(6-(1- (difluoromethyl)-1H-1,2,4-triazol-3- yl)-2-(2-fluorophenyl)-1H-imidazo [4,5-c]pyridin-1-yl)cyclohexyl)-2- (2-hydroxyethoxy)benzamide |
| 301 | 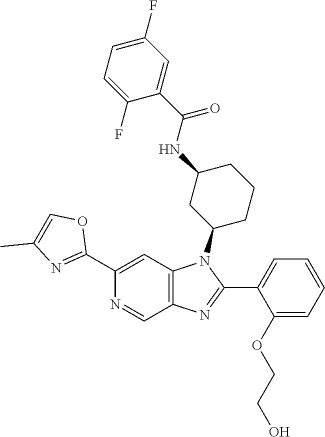 [SMILES: Cc1coc(-c2cc3c(cn2)nc(-c2ccccc2OCCO)n3[C@@H]2CCC[C@H](NC(=O)c3cc(F)ccc3F)C2)n1] | 2,5-difluoro-N-((1S,3R)-3-(2-(2-(2- hydroxyethoxy)phenyl)-6-(4-meth- yloxazol-2-yl)-1H-imidazo[4,5-c] pyridin-1-yl)cyclohexyl)benzamide |
| 302 | 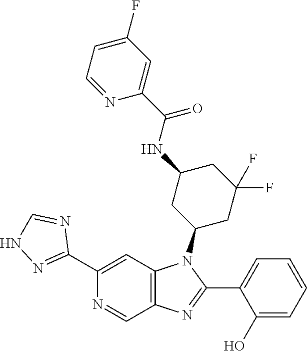 [SMILES: O=C(N[C@@H]1C[C@H](n2c(-c3ccccc3O)nc3cnc(-c4nc[nH]n4)cc32)CC(F)(F)C1)c1cc(F)ccn1] | N-((1R,5S)-3,3-difluoro-5-(2-(2- hydroxyphenyl)-6-(1H-1,2,4-triazol- 3-yl)-1H-imidazo[4,5-c]pyridin-1- yl)cyclohexyl)-4-fluoropicolinamide |
| 303 | 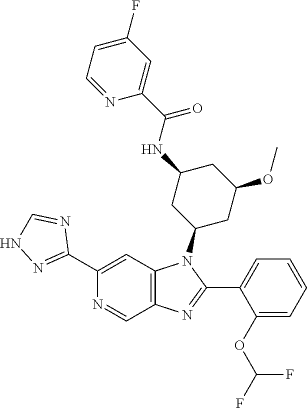 [SMILES: CO[C@@H]1C[C@H](NC(=O)c2cc(F)ccn2)C[C@H](n2c(-c3ccccc3OC(F)F)nc3cnc(-c4nc[nH]n4)cc32)C1] | N-((1R,3S,5R)-3-(2-(2-(difluoro- methoxy)phenyl)-6-(1H-1,2,4- triazol-3-yl)-1H-imidazo[4,5-c] pyridin-1-yl)-5-methoxycyclo- hexyl)-4-fluoropicolinamide |
| 304 | 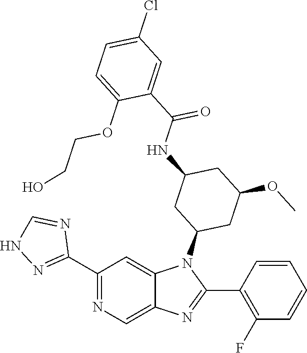 [SMILES: CO[C@@H]1C[C@H](NC(=O)c2cc(Cl)ccc2OCCO)C[C@H](n2c(-c3ccccc3F)nc3cnc(-c4nc[nH]n4)cc32)C1] | 5-chloro-N-((1R,3S,5R)-3-(2-(2- fluorophenyl)-6-(1H-1,2,4-triazol- 3-yl)-1H-imidazo[4,5-c]pyridin-1- yl)-5-methoxycyclohexyl)-2-(2- hydroxyethoxy)benzamide |
| 305 | 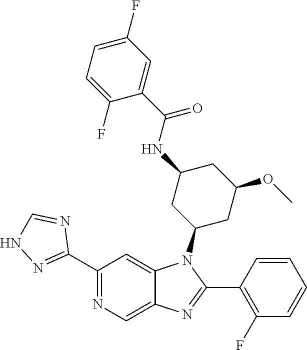 [SMILES: CO[C@@H]1C[C@H](NC(=O)c2cc(F)ccc2F)C[C@H](n2c(-c3ccccc3F)nc3cnc(-c4nc[nH]n4)cc32)C1] | 2,5-difluoro-N-((1R,3S,5R)-3-(2-(2- fluorophenyl)-6-(1H-1,2,4-triazol-3- yl)-1H-imidazo[4,5-c]pyridin-1-yl)- 5-methoxycyclohexyl)benzamide |
| 306 | 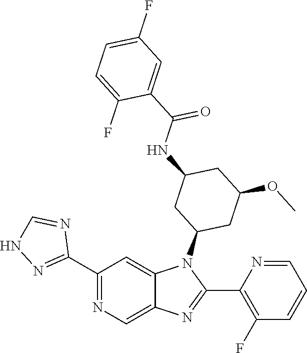 [SMILES: CO[C@@H]1C[C@H](NC(=O)c2cc(F)ccc2F)C[C@H](n2c(-c3ncccc3F)nc3cnc(-c4nc[nH]n4)cc32)C1] | 2,5-difluoro-N-((1R,3S,5R)-3-(2- (3-fluoropyridin-2-yl)-6-(1H-1,2,4- triazol-3-yl)-1H-imidazo[4,5-c] pyridin-1-yl)-5-methoxycyclohex- yl)benzamide |
| 307 | 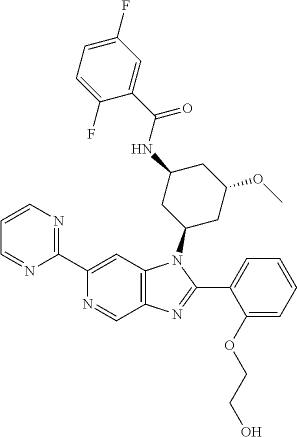 [SMILES: CO[C@H]1C[C@H](NC(=O)c2cc(F)ccc2F)C[C@H](n2c(-c3ccccc3OCCO)nc3cnc(-c4ncccn4)cc32)C1] | 2,5-difluoro-N-((1R,3S,5S)-3-(2-(2- (2-hydroxyethoxy)phenyl)-6- (pyrimidin-2-yl)-1H-imidazo[4,5-c] pyridin-1-yl)-5-methoxycyclohex- yl)benzamide |
| 308 | 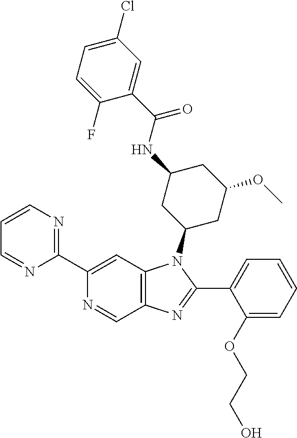 [SMILES: CO[C@H]1C[C@H](NC(=O)c2cc(Cl)ccc2F)C[C@H](n2c(-c3ccccc3OCCO)nc3cnc(-c4ncccn4)cc32)C1] | 5-chloro-2-fluoro-N-((1R,3S,5S)-3- (2-(2-(2-hydroxyethoxy)phenyl)-6- (pyrimidin-2-yl)-1H-imidazo[4,5-c] pyridin-1-yl)-5-methoxycyclohex- yl)benzamide |
| 309 | 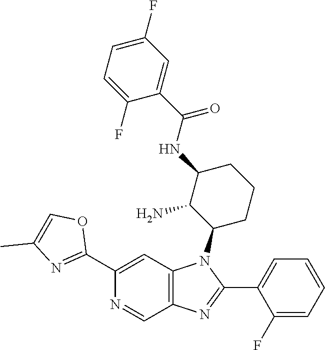 [SMILES: Cc1coc(-c2cc3c(cn2)nc(-c2ccccc2F)n3[C@@H]2CCC[C@H](NC(=O)c3cc(F)ccc3F)[C@H]2N)n1] | N-((1S,2R,3R)-2-amino-3-(2-(2- fluorophenyl)-6-(4-methyloxazol-2- yl)-1H-imidazo[4,5-c]pyridin-1-yl)- cyclohexyl)-2,5-difluorobenzamide |
| 310 | 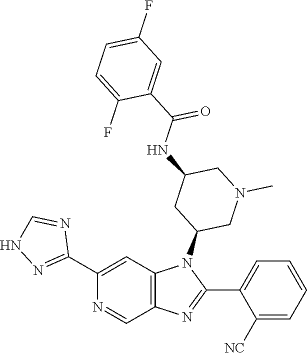 [SMILES: CN1C[C@H](NC(=O)c2cc(F)ccc2F)C[C@H](n2c(-c3ccccc3C#N)nc3cnc(-c4nc[nH]n4)cc32)C1] | 2,5-difluoro-N-((3R,5S)-5-(2-(2- cyanophenyl)-6-(1H-1,2,4-triazol-3- yl)-1H-imidazo[4,5-c]pyridin-1-yl)- 1-methylpiperidin-3-yl)benzamide |
| 311 | 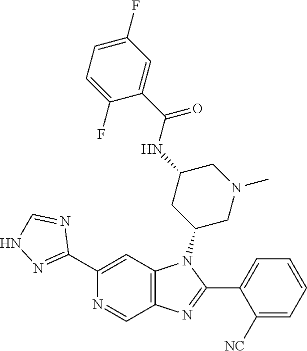 [SMILES: CN1C[C@@H](NC(=O)c2cc(F)ccc2F)C[C@@H](n2c(-c3ccccc3C#N)nc3cnc(-c4nc[nH]n4)cc32)C1] | 2,5-difluoro-N-((3S,5R)-5-(2-(2- cyanophenyl)-6-(1H-1,2,4-triazol- 3-yl)-1H-imidazo[4,5-c]pyridin-1- yl)-1-methylpiperidin-3-yl)benz- amide |
| 312 | 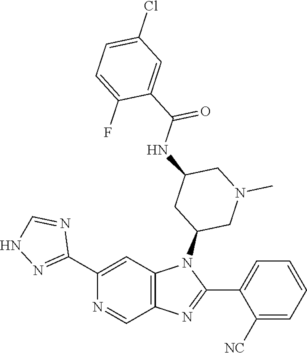 [SMILES: CN1C[C@H](NC(=O)c2cc(Cl)ccc2F)C[C@H](n2c(-c3ccccc3C#N)nc3cnc(-c4nc[nH]n4)cc32)C1] | 5-chloro-2-fluoro-N-((3R,5S)-5- (2-(2-cyanophenyl)-6-(1H-1,2,4- triazol-3-yl)-1H-imidazo[4,5-c] pyridin-1-yl)-1-methylpiperidin- 3-yl)benzamide |
| 313 | 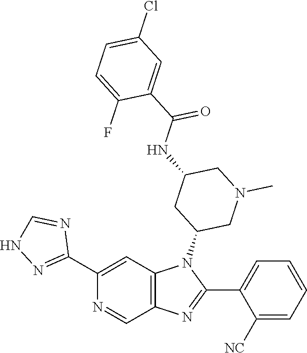 [SMILES: CN1C[C@@H](NC(=O)c2cc(Cl)ccc2F)C[C@@H](n2c(-c3ccccc3C#N)nc3cnc(-c4nc[nH]n4)cc32)C1] | 5-chloro-2-fluoro-N-((3S,5R)-5- (2-(2-cyanophenyl)-6-(1H-1,2,4- triazol-3-yl)-1H-imidazo[4,5-c] pyridin-1-yl)-1-methylpiperidin- 3-yl)benzamide |
| 314 | 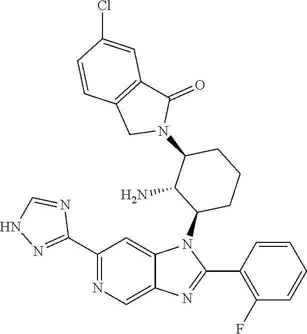 [SMILES: N[C@H]1[C@H](n2c(-c3ccccc3F)nc3cnc(-c4nc[nH]n4)cc32)CCC[C@@H]1N1Cc2ccc(Cl)cc2C1=O] | 2-((1S,2S,3R)-2-amino-3-(2-(2- fluorophenyl)-6-(1H-1,2,4-triazol- 3-yl)-1H-imidazo[4,5-c]pyridin-1- yl)cyclohexyl)-6-chloroisoindolin- 1-one |
| 315 | 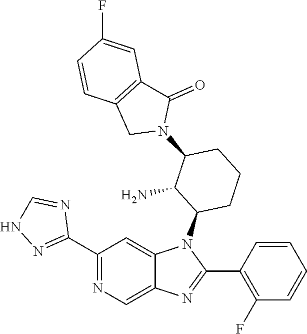 [SMILES: N[C@H]1[C@H](n2c(-c3ccccc3F)nc3cnc(-c4nc[nH]n4)cc32)CCC[C@@H]1N1Cc2ccc(F)cc2C1=O] | 2-((1S,2S,3R)-2-amino-3-(2-(2- fluorophenyl)-6-(1H-1,2,4-triazol- 3-yl)-1H-imidazo[4,5-c]pyridin-1- yl)cyclohexyl)-6-fluoroisoindolin- 1-one |
| 316 | 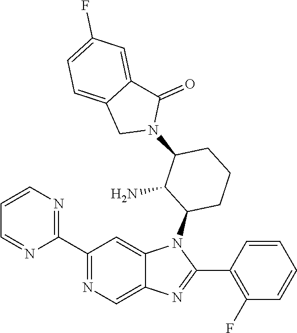 [SMILES: N[C@H]1[C@H](n2c(-c3ccccc3F)nc3cnc(-c4ncccn4)cc32)CCC[C@@H]1N1Cc2ccc(F)cc2C1=O] | 2-((1S,2S,3R)-2-amino-3-(2-(2- fluorophenyl)-6-(pyrimidin-2-yl)- 1H-imidazo[4,5-c]pyridin-1-yl)- cyclohexyl)-6-fluoroisoindolin-1- one |
| 317 | 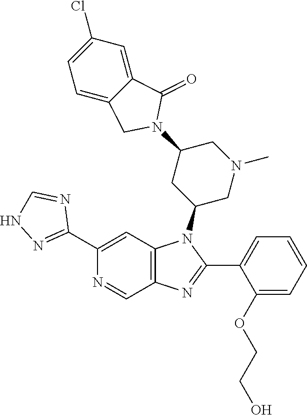 [SMILES: CN1C[C@H](N2Cc3ccc(Cl)cc3C2=O)C[C@H](n2c(-c3ccccc3OCCO)nc3cnc(-c4nc[nH]n4)cc32)C1] | 6-chloro-2-((3R,5S)-5-(2-(2-(2- hydroxyethoxy)phenyl)-6-(1H- 1,2,4-triazol-3-yl)-1H-imidazo [4,5-c]pyridin-1-yl)-1-methyl- piperidin-3-yl)isoindolin-1-one |
| 318 | 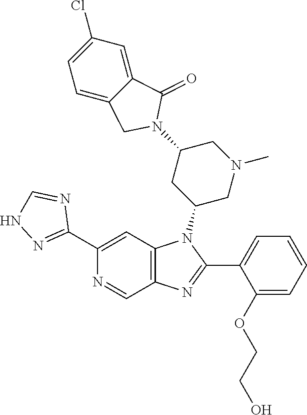 [SMILES: CN1C[C@@H](N2Cc3ccc(Cl)cc3C2=O)C[C@@H](n2c(-c3ccccc3OCCO)nc3cnc(-c4nc[nH]n4)cc32)C1] | 6-chloro-2-((3S,5R)-5-(2-(2-(2- hydroxyethoxy)phenyl)-6-(1H- 1,2,4-triazol-3-yl)-1H-imidazo [4,5-c]pyridin-1-yl)-1-methyl- piperidin-3-yl)isoindolin-1-one |
| 319 | 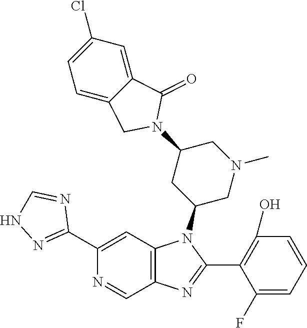 [SMILES: CN1C[C@H](N2Cc3ccc(Cl)cc3C2=O)C[C@H](n2c(-c3c(O)cccc3F)nc3cnc(-c4nc[nH]n4)cc32)C1] | 6-chloro-2-((3R,5S)-5-(2-(2-fluoro- 6-hydroxyphenyl)-6-(1H-1,2,4- triazol-3-yl)-1H-imidazo[4,5-c] pyridin-1-yl)-1-methylpiperidin- 3-yl)isoindolin-1-one |
| 320 | 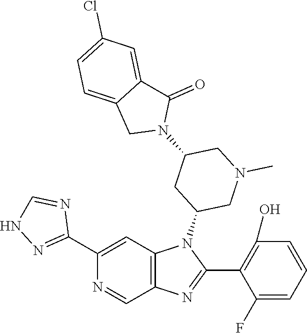 [SMILES: CN1C[C@@H](N2Cc3ccc(Cl)cc3C2=O)C[C@@H](n2c(-c3c(O)cccc3F)nc3cnc(-c4nc[nH]n4)cc32)C1] | 6-chloro-2-((3S,5R)-5-(2-(2-fluoro- 6-hydroxyphenyl)-6-(1H-1,2,4- triazol-3-yl)-1H-imidazo[4,5-c] pyridin-1-yl)-1-methylpiperidin- 3-yl)isoindolin-1-one |
| 321 | 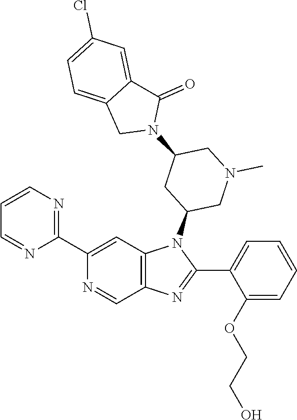 [SMILES: CN1C[C@H](N2Cc3ccc(Cl)cc3C2=O)C[C@H](n2c(-c3ccccc3OCCO)nc3cnc(-c4ncccn4)cc32)C1] | 6-chloro-2-((3R,5S)-5-(2-(2-(2- hydroxyethoxy)phenyl)-6-(pyr- imidin-2-yl)-1H-imidazo[4,5-c] pyridin-1-yl)-1-methylpiperidin- 3-yl)isoindolin-1-one |
| 322 | 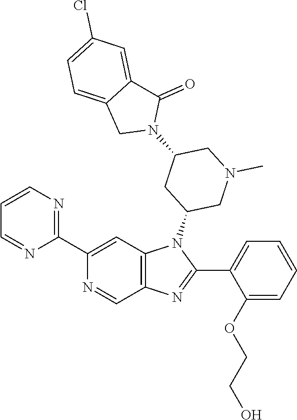 [SMILES: CN1C[C@@H](N2Cc3ccc(Cl)cc3C2=O)C[C@@H](n2c(-c3ccccc3OCCO)nc3cnc(-c4ncccn4)cc32)C1] | 6-chloro-2-((3S,5R)-5-(2-(2-(2- hydroxyethoxy)phenyl)-6-(pyrimi- din-2-yl)-1H-imidazo[4,5-c]pyri- din-1-yl)-1-methylpiperidin-3-yl)- isoindolin-1-one |
| 323 | 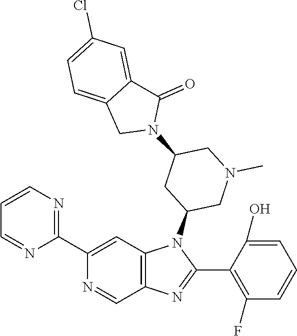 [SMILES: CN1C[C@H](N2Cc3ccc(Cl)cc3C2=O)C[C@H](n2c(-c3c(O)cccc3F)nc3cnc(-c4ncccn4)cc32)C1] | 6-chloro-2-((3R,5S)-5-(2-(2-fluoro- 6-hydroxyphenyl)-6-(pyrimidin-2- yl)-1H-imidazo[4,5-c]pyridin-1-yl)- 1-methylpiperidin-3-yl)isoindolin- 1-one |
| 324 | 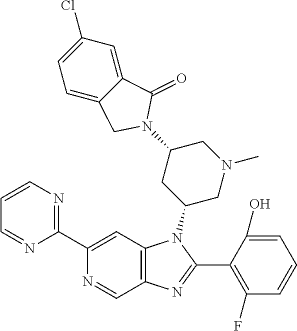 [SMILES: CN1C[C@@H](N2Cc3ccc(Cl)cc3C2=O)C[C@@H](n2c(-c3c(O)cccc3F)nc3cnc(-c4ncccn4)cc32)C1] | 6-chloro-2-((3S,5R)-5-(2-(2-fluoro- 6-hydroxyphenyl)-6-(pyrimidin-2- yl)-1H-imidazo[4,5-c]pyridin-1-yl)- 1-methylpiperidin-3-yl)isoindolin- 1-one |
| 325 | 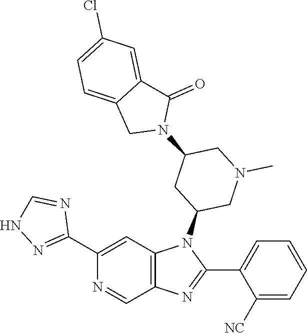 [SMILES: CN1C[C@H](N2Cc3ccc(Cl)cc3C2=O)C[C@H](n2c(-c3ccccc3C#N)nc3cnc(-c4nc[nH]n4)cc32)C1] | 2-(1-((3S,5R)-5-(6-chloro-1-oxo- isoindolin-2-yl)-1-methylpiperidin- 3-yl)-6-(1H-1,2,4-triazol-3-yl)-1H- imidazo[4,5-c]pyridin-2-yl)benzo- nitrile |
| 326 | 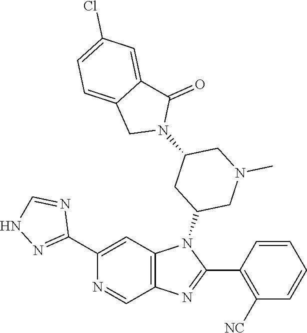 | 2-(1-((3R,5S)-5-(6-chloro-1-oxo- isoindolin-2-yl)-1-methylpiperidin- 3-yl)-6-(1H-1,2,4-triazol-3-yl)-1H- imidazo[4,5-c]pyridin-2-yl)benzo- nitrile |
| 327 | 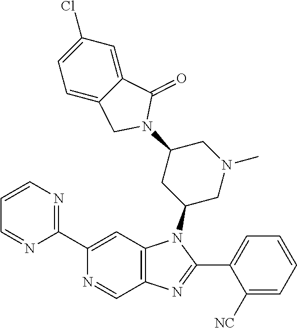 [SMILES: CN1C[C@H](N2Cc3ccc(Cl)cc3C2=O)C[C@H](n2c(-c3ccccc3C#N)nc3cnc(-c4ncccn4)cc32)C1] | 2-(1-((3S,5R)-5-(6-chloro-1-oxo- isoindolin-2-yl)-1-methylpiperidin- 3-yl)-6-(pyrimidin-2-yl)-1H- imidazo[4,5-c]pyridin-2-yl)benzo- nitrile |
| 328 | 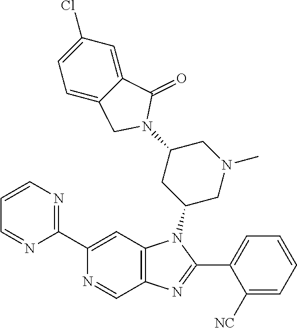 [SMILES: CN1C[C@@H](N2Cc3ccc(Cl)cc3C2=O)C[C@@H](n2c(-c3ccccc3C#N)nc3cnc(-c4ncccn4)cc32)C1] | 2-(1-((3R,5S)-5-(6-chloro-1-oxo- isoindolin-2-yl)-1-methylpiperidin- 3-yl)-6-(pyrimidin-2-yl)-1H- imidazo[4,5-c]pyridin-2-yl)benzo- nitrile |
| 329 | 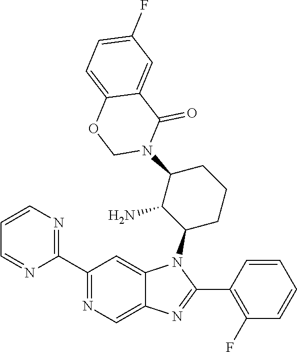 [SMILES: N[C@H]1[C@H](n2c(-c3ccccc3F)nc3cnc(-c4ncccn4)cc32)CCC[C@@H]1N1COc2ccc(F)cc2C1=O] | 3-((1S,2S,3R)-2-amino-3-(2-(2- fluorophenyl)-6-(pyrimidin-2-yl)- 1H-imidazo[4,5-c]pyridin-1-yl)- cyclohexyl)-6-fluoro-2,3-dihydro- 4H-benzo[e][1,3]oxazin-4-one |

In some embodiments, the disclosure provides specific examples of Formula II, and their pharmaceutically acceptable salts and/or isotopologues, as set forth in Table 3 below.

TABLE 3

| Ex. | | |
|---|---|---|
| No. | Structure | Name |
| 217 | 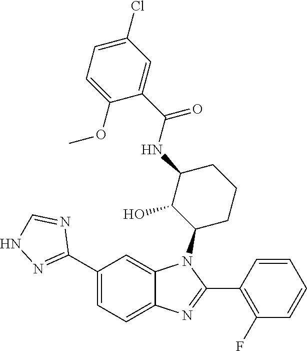 [SMILES: COc1ccc(Cl)cc1C(=O)N[C@H]1CCC[C@@H](n2c(-c3ccccc3F)nc3ccc(-c4nc[nH]n4)cc32)[C@@H]1O] | 5-chloro-N-((1S,2R,3R)-3-(2- (2-fluorophenyl)-6-(1H-1,2,4- triazol-3-yl)-1H-benzo[d] imidazol-1-yl)-2-hydroxycyclo- hexyl)-2-methoxybenzamide |
| 218 | 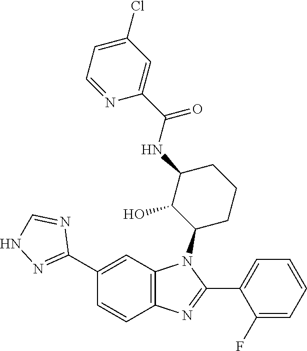 [SMILES: O=C(N[C@H]1CCC[C@@H](n2c(-c3ccccc3F)nc3ccc(-c4nc[nH]n4)cc32)[C@@H]1O)c1cc(Cl)ccn1] | 4-chloro-N-((1S,2R,3R)-3-(2-(2- fluorophenyl)-6-(1H-1,2,4- triazol-3-yl)-1H-benzo[d]imid- azol-1-yl)-2-hydroxycyclohex- yl)picolinamide |
| 219 | 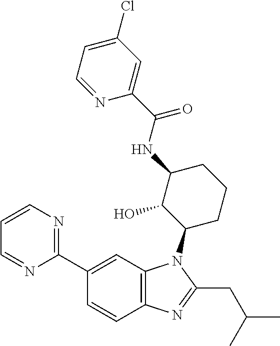 [SMILES: CC(C)Cc1nc2ccc(-c3ncccn3)cc2n1[C@@H]1CCC[C@H](NC(=O)c2cc(Cl)ccn2)[C@H]1O] | 4-chloro-N-((1S,2R,3R)-2- hydroxy-3-(2-isobutyl-6-(pyrim- idin-2-yl)-1H-benzo[d]imidazol- 1-yl)cyclohexyl)picolinamide |
| 220 | 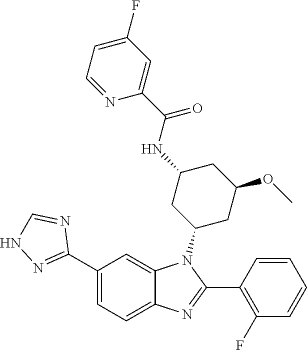 [SMILES: CO[C@@H]1C[C@@H](NC(=O)c2cc(F)ccn2)C[C@@H](n2c(-c3ccccc3F)nc3ccc(-c4nc[nH]n4)cc32)C1] | 4-fluoro-N-((1R,3S,5S)-3-(2-(2- fluorophenyl)-6-(1H-1,2,4- triazol-3-yl)-1H-benzo[d]imid- azol-1-yl)-5-methoxycyclohex- yl)picolinamide |
| 221 | 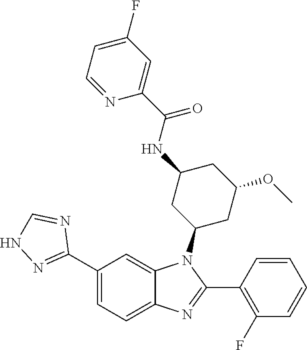 [SMILES: CO[C@H]1C[C@H](NC(=O)c2cc(F)ccn2)C[C@H](n2c(-c3ccccc3F)nc3ccc(-c4nc[nH]n4)cc32)C1] | 4-fluoro-N-((1S,3R,5R)-3-(2-(2- fluorophenyl)-6-(1H-1,2,4- triazol-3-yl)-1H-benzo[d]imid- azol-1-yl)-5-methoxycyclohex- yl)picolinamide |
| 222 | 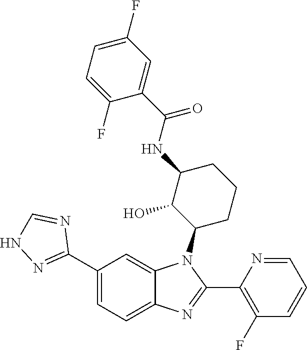 [SMILES: O=C(N[C@H]1CCC[C@@H](n2c(-c3ncccc3F)nc3ccc(-c4nc[nH]n4)cc32)[C@@H]1O)c1cc(F)ccc1F] | 2,5-difluoro-N-((1S,2R,3R)-3- (2-(3-fluoropyridin-2-yl)-6-(1H- 1,2,4-triazol-3-yl)-1H-benzo[d] imidazol-1-yl)-2-hydroxycyclo- hexyl)benzamide |
| 223 | 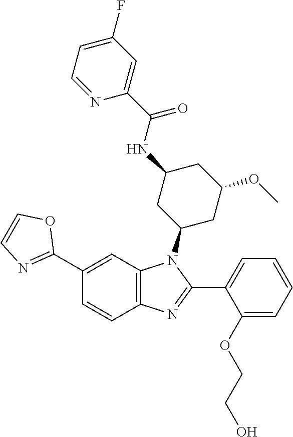 [SMILES: CO[C@H]1C[C@H](NC(=O)c2cc(F)ccn2)C[C@H](n2c(-c3ccccc3OCCO)nc3ccc(-c4ncco4)cc32)C1] | 4-fluoro-N-((1R,3S,5S)-3-(2-(2- (2-hydroxyethoxy)phenyl)-6- (oxazol-2-yl)-1H-benzo[d]imid- azol-1-yl)-5-methoxycyclohex- yl)picolinamide |
| 224 | 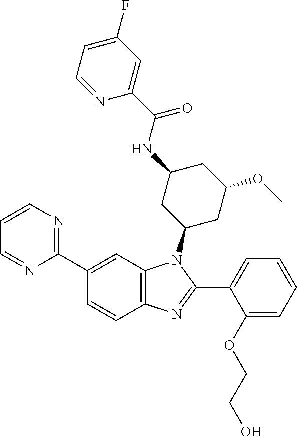 [SMILES: CO[C@H]1C[C@H](NC(=O)c2cc(F)ccn2)C[C@H](n2c(-c3ccccc3OCCO)nc3ccc(-c4ncccn4)cc32)C1] | 4-fluoro-N-((1R,3S,5S)-3-(2-(2- (2-hydroxyethoxy)phenyl)-6- (pyrimidin-2-yl)-1H-benzo[d] imidazol-1-yl)-5-methoxycyclo- hexyl)picolinamide |
| 225 | 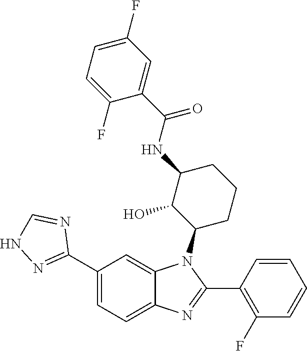 [SMILES: O=C(N[C@H]1CCC[C@@H](n2c(-c3ccccc3F)nc3ccc(-c4nc[nH]n4)cc32)[C@@H]1O)c1cc(F)ccc1F] | 2,5-difluoro-N-((1S,2R,3R)-3- (2-(2-fluorophenyl)-6-(1H-1,2,4- triazol-3-yl)-1H-benzo[d]imid- azol-1-yl)-2-hydroxycyclohex- yl)benzamide |
| 226 | 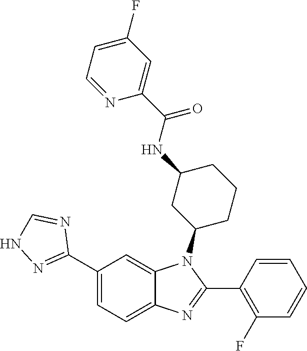 [SMILES: O=C(N[C@H]1CCC[C@@H](n2c(-c3ccccc3F)nc3ccc(-c4nc[nH]n4)cc32)C1)c1cc(F)ccn1] | 4-fluoro-N-((1S,3R)-3-(2-(2- fluorophenyl)-6-(1H-1,2,4- triazol-3-yl)-1H-benzo[d]imid- azol-1-yl)cyclohexyl)picolin- amide |
| 227 | 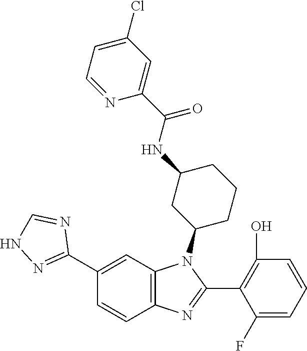 [SMILES: O=C(N[C@H]1CCC[C@@H](n2c(-c3c(O)cccc3F)nc3ccc(-c4nc[nH]n4)cc32)C1)c1cc(Cl)ccn1] | 4-chloro-N-((1S,3R)-3-(2-(2- fluoro-6-hydroxyphenyl)-6-(1H- 1,2,4-triazol-3-yl)-1H-benzo[d] imidazol-1-l)cyclohexyl)picolin- amide |
| 228 | 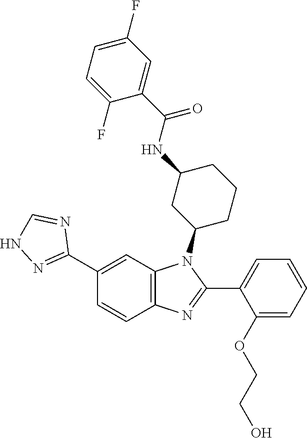 [SMILES: O=C(N[C@H]1CCC[C@@H](n2c(-c3ccccc3OCCO)nc3ccc(-c4nc[nH]n4)cc32)C1)c1cc(F)ccc1F] | 2,5-difluoro-N-((1S,3R)-3-(2- (2-(2-hydroxyethoxy)phenyl)- 6-(1H-1,2,4-triazol-3-yl)-1H- benzo[d]imidazol-1-yl)cyclo- hexyl)benzamide |
| 229 | 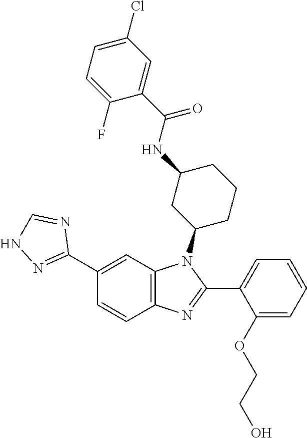 [SMILES: O=C(N[C@H]1CCC[C@@H](n2c(-c3ccccc3OCCO)nc3ccc(-c4nc[nH]n4)cc32)C1)c1cc(Cl)ccc1F] | 5-chloro-2-fluoro-N-((1S,3R)-3- (2-(2-(2-hydroxyethoxy)phenyl)- 6-(1H-1,2,4-triazol-3-yl)-1H- benzo[d]imidazol-1-yl)cyclohex- yl)benzamide |
| 230 | 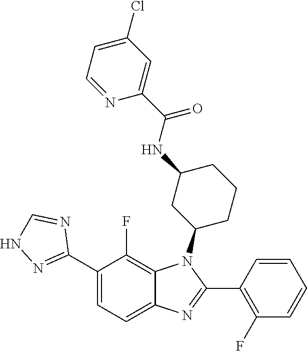 [SMILES: O=C(N[C@H]1CCC[C@@H](n2c(-c3ccccc3F)nc3ccc(-c4nc[nH]n4)c(F)c32)C1)c1cc(Cl)ccn1] | 4-chloro-N-((1S,3R)-3-(7-fluoro- 2-(2-fluorophenyl)-6-(1H-1,2,4- triazol-3-yl)-1H-benzo[d]imid- azol-1-yl)cyclohexyl)picolin- amide |
| 231 | 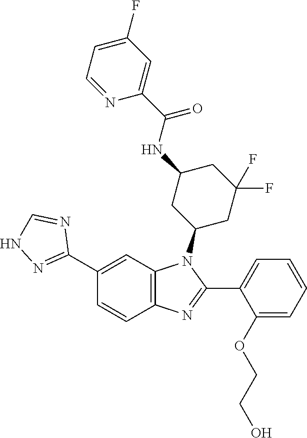 [SMILES: O=C(N[C@@H]1C[C@H](n2c(-c3ccccc3OCCO)nc3ccc(-c4nc[nH]n4)cc32)CC(F)(F)C1)c1cc(F)ccn1] | N-((1R,5S)-3,3-difluoro-5-(2- (2-(2-hydroxyethoxy)phenyl)- 6-(1H-1,2,4-triazol-3-yl)-1H- benzo[d]imidazol-1-yl)cyclo- hexyl)-4-fluoropicolinamide |
| 232 | 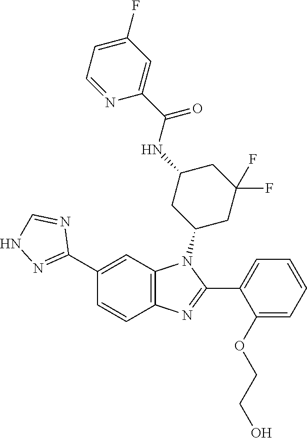 [SMILES: O=C(N[C@H]1C[C@@H](n2c(-c3ccccc3OCCO)nc3ccc(-c4nc[nH]n4)cc32)CC(F)(F)C1)c1cc(F)ccn1] | N-((1S,5R)-3,3-difluoro-5-(2- (2-(2-hydroxyethoxy)phenyl)-6- (1H-1,2,4-triazol-3-yl)-1H-benzo [d]imidazol-1-yl)cyclohexyl)-4- fluoropicolinamide |
| 233 | 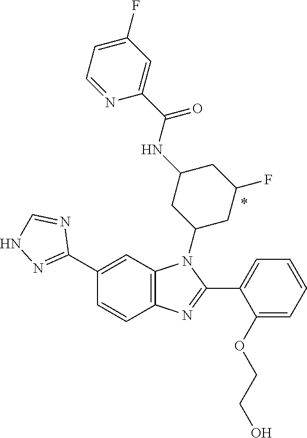 [SMILES: O=C(NC1CC(F)CC(n2c(-c3ccccc3OCCO)nc3ccc(-c4nc[nH]n4)cc32)C1)c1cc(F)ccn1] | 4-fluoro-N-(3-fluoro-5-(2-(2-(2- hydroxyethoxy)phenyl)-6-(1H- 1,2,4-triazol-3-yl)-1H-benzo[d] imidazol-1-yl)cyclohexyl)- picolinamide⁽⁵⁾ ⁽⁵⁾relative stereochemistry at centers 1 and 5 of cyclohexyl ring is cis with the stereo- chemistry at center 3 fixed but undetermined |
| 234 | 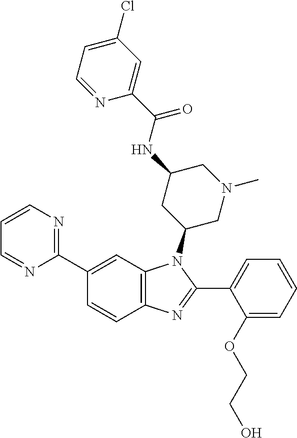 [SMILES: CN1C[C@H](NC(=O)c2cc(Cl)ccn2)C[C@H](n2c(-c3ccccc3OCCO)nc3ccc(-c4ncccn4)cc32)C1] | 4-chloro-N-((3R,5S)-5-(2-(2-(2- hydroxyethoxy)phenyl)-6-(pyr- imidin-2-yl)-1H-benzo[d]imid- azol-1-yl)-1-methylpiperidin-3- yl)picolinamide |
| 235 | 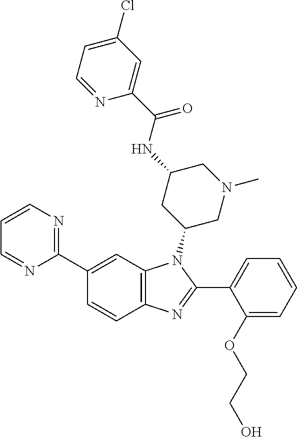 [SMILES: CN1C[C@@H](NC(=O)c2cc(Cl)ccn2)C[C@@H](n2c(-c3ccccc3OCCO)nc3ccc(-c4ncccn4)cc32)C1] | 4-chloro-N-((3S,5R)-5-(2-(2-(2- hydroxyethoxy)phenyl)-6-(pyr- imidin-2-yl)-1H-benzo[d]imid- azol-1-yl)-1-methylpiperidin-3- yl)picolinamide |
| 236 | 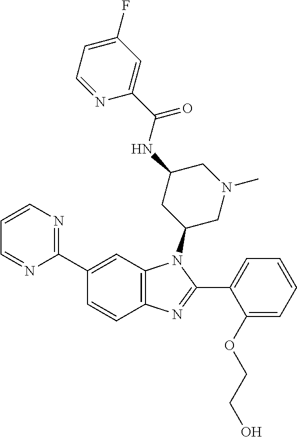 [SMILES: CN1C[C@H](NC(=O)c2cc(F)ccn2)C[C@H](n2c(-c3ccccc3OCCO)nc3ccc(-c4ncccn4)cc32)C1] | 4-fluoro-N-((3R,5S)-5-(2-(2-(2- hydroxyethoxy)phenyl)-6-(pyr- imidin-2-yl)-1H-benzo[d]imid- azol-1-yl)-1-methylpiperidin-3- yl)picolinamide |
| 237 | 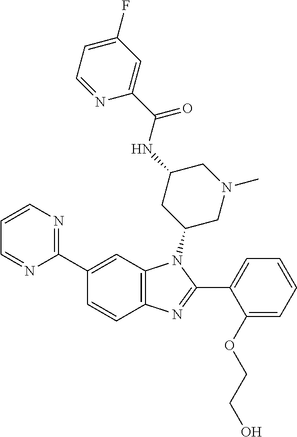 [SMILES: CN1C[C@@H](NC(=O)c2cc(F)ccn2)C[C@@H](n2c(-c3ccccc3OCCO)nc3ccc(-c4ncccn4)cc32)C1] | 4-fluoro-N-((3S,5R)-5-(2-(2-(2- hydroxyethoxy)phenyl)-6-(pyr- imidin-2-yl)-1H-benzo[d]imid- azol-1-yl)-1-methylpiperidin-3- yl)picolinamide |
| 238 | 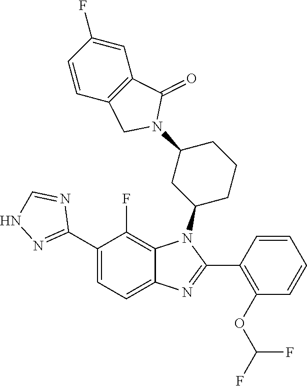 [SMILES: O=C1c2cc(F)ccc2CN1[C@H]1CCC[C@@H](n2c(-c3ccccc3OC(F)F)nc3ccc(-c4nc[nH]n4)c(F)c32)C1] | 2-((1S,3R)-3-(2-(2-(difluoro- methoxy)phenyl)-7-fluoro-6- (1H-1,2,4-triazol-3-yl)-1H- benzo[d]imidazol-1-yl)cyclo- hexyl)-6-fluoroisoindolin-1- one |
| 239 | 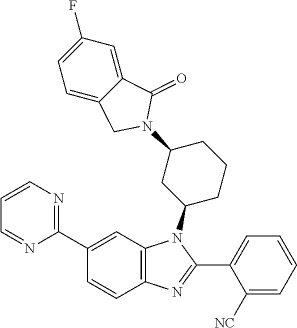 [SMILES: N#Cc1ccccc1-c1nc2ccc(-c3ncccn3)cc2n1[C@@H]1CCC[C@H](N2Cc3ccc(F)cc3C2=O)C1] | 2-(1-((1R,3S)-3-(6-fluoro-1- oxoisoindolin-2-yl)cyclohexyl)- 6-(pyrimidin-2-yl)-1H-benzo[d] imidazol-2-yl)benzonitrile |
| 240 | 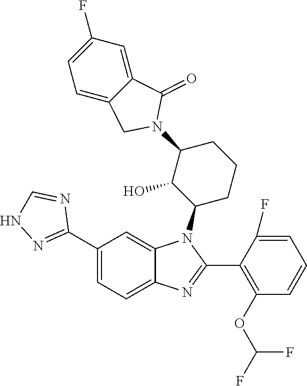 [SMILES: O=C1c2cc(F)ccc2CN1[C@H]1CCC[C@@H](n2c(-c3c(F)cccc3OC(F)F)nc3ccc(-c4nc[nH]n4)cc32)[C@@H]1O] | 2-((1S,2S,3R)-3-(2-(2-(difluoro- methoxy)-6-fluorophenyl)-6- (1H-1,2,4-triazol-3-yl)-1H-benzo [d]imidazol-1-yl)-2-hydroxy- cyclohexyl)-6-fluoroisoindolin- 1-one |
| 241 | 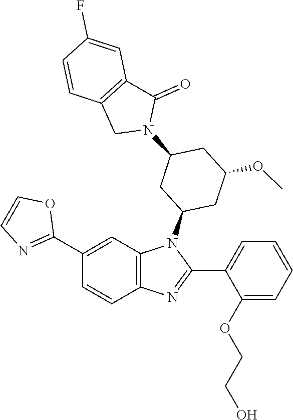 [SMILES: CO[C@H]1C[C@H](N2Cc3ccc(F)cc3C2=O)C[C@H](n2c(-c3ccccc3OCCO)nc3ccc(-c4ncco4)cc32)C1] | 6-fluoro-2-((1R,3S,5R)-3-(2-(2- (2-hydroxyethoxy)phenyl)-6- (oxazol-2-yl)-1H-benzo[d]imid- azol-1-yl)-5-methoxycyclohex- yl)isoindolin-1-one |
| 242 | 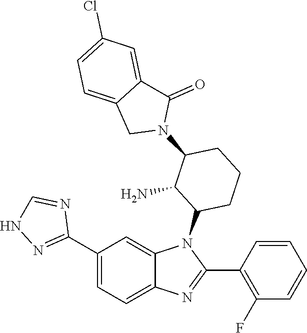 | 2-((1S,2S,3R)-2-amino-3-(2- (2-fluorophenyl)-6-(1H-1,2,4- triazol-3-yl)-1H-benzo[d] imidazol-1-yl)cyclohexyl)- 6-chloroisoindolin-1-one |
| 243 | 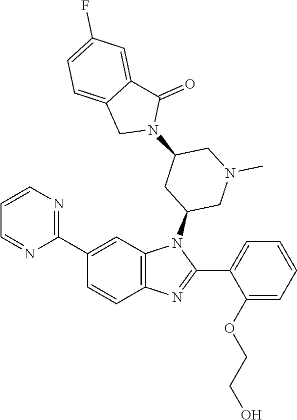 [SMILES: CN1C[C@H](N2Cc3ccc(F)cc3C2=O)C[C@H](n2c(-c3ccccc3OCCO)nc3ccc(-c4ncccn4)cc32)C1] | 6-fluoro-2-((3R,5S)-5-(2-(2- (2-hydroxyethoxy)phenyl)-6- (pyrimidin-2-yl)-1H-benzo[d] imidazol-1-yl)-1-methylpiper- idin-3-yl)isoindolin-1-one |
| 244 | 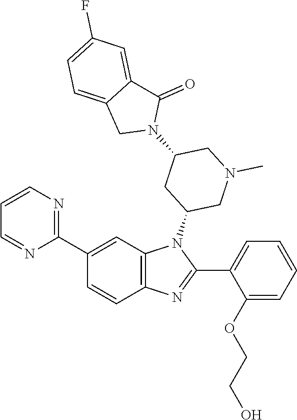 [SMILES: CN1C[C@@H](N2Cc3ccc(F)cc3C2=O)C[C@@H](n2c(-c3ccccc3OCCO)nc3ccc(-c4ncccn4)cc32)C1] | 6-fluoro-2-((3S,5R)-5-(2-(2-(2- hydroxyethoxy)phenyl)-6-(pyr- imidin-2-yl)-1H-benzo[d]imid- azol-1-yl)-1-methylpiperidin-3- yl)isoindolin-1-one |

In some embodiments, the disclosure provides additional prophetic examples of Formula II, and their pharmaceutically acceptable salts and/or isotopologues, as set forth in Table 4 below. The prophetic compounds in Table 4 may be made in accordance with the procedures described in the General Schemes, Intermediate Schemes, and Examples herein alone or in combination with knowledge of a person of ordinary skill in the art.

TABLE 4

| Ex. | | |
|---|---|---|
| No. | Structure | Name |
| 330 | 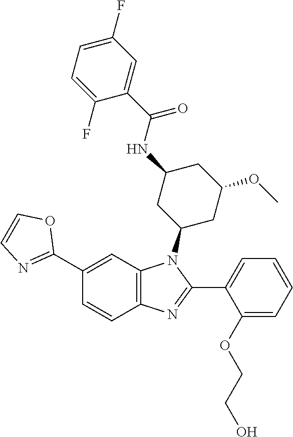 [SMILES: CO[C@H]1C[C@H](NC(=O)c2cc(F)ccc2F)C[C@H](n2c(-c3ccccc3OCCO)nc3ccc(-c4ncco4)cc32)C1] | 2,5-difluoro-N-((1R,3S,5S)- 3-(2-(2-(2-hydroxyethoxy)- phenyl)-6-(oxazol-2-yl)-1H- benzo[d]imidazol-1-yl)-5- methoxycyclohexyl)benz- amide |
| 331 | 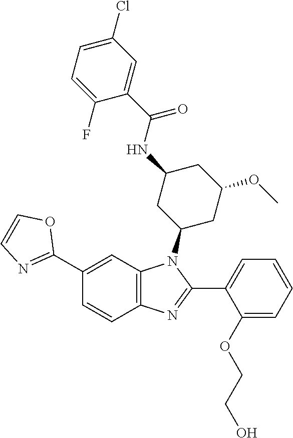 [SMILES: CO[C@H]1C[C@H](NC(=O)c2cc(Cl)ccc2F)C[C@H](n2c(-c3ccccc3OCCO)nc3ccc(-c4ncco4)cc32)C1] | 5-chloro-2-fluoro-N-((1R, 3S,5S)-3-(2-(2-(2-hydroxy- ethoxy)phenyl)-6-(oxazol-2- yl)-1H-benzo[d]imidazol-1- yl)-5-methoxycyclohexyl)- benzamide |
| 332 | 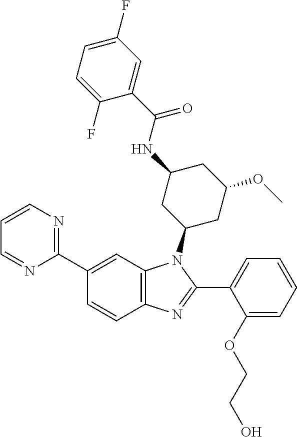 [SMILES: CO[C@H]1C[C@H](NC(=O)c2cc(F)ccc2F)C[C@H](n2c(-c3ccccc3OCCO)nc3ccc(-c4ncccn4)cc32)C1] | 2,5-difluoro-N-((1R,3S,5S)- 3-(2-(2-(2-hydroxyethoxy)- phenyl)-6-(pyrimidin-2-yl)- 1H-benzo[d]imidazol-1-yl)- 5-methoxycyclohexyl)benz- amide |
| 333 | 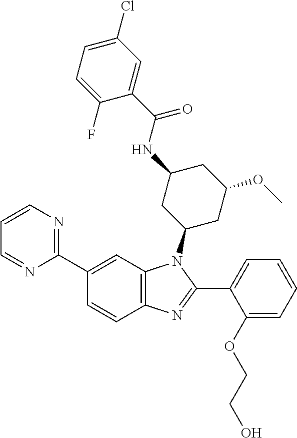 [SMILES: CO[C@H]1C[C@H](NC(=O)c2cc(Cl)ccc2F)C[C@H](n2c(-c3ccccc3OCCO)nc3ccc(-c4ncccn4)cc32)C1] | 5-chloro-2-fluoro-N-((1R, 3S,5S)-3-(2-(2-(2-hydroxy- ethoxy)phenyl)-6-(pyrimi- din-2-yl)-1H-benzo[d]imid- azol-1-yl)-5-methoxycyclo- hexyl)benzamide |
| 334 | 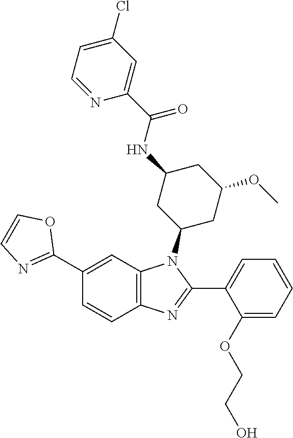 [SMILES: CO[C@H]1C[C@H](NC(=O)c2cc(Cl)ccn2)C[C@H](n2c(-c3ccccc3OCCO)nc3ccc(-c4ncco4)cc32)C1] | 4-chloro-N-((1R,3S,5S)-3- (2-(2-(2-hydroxyethoxy)- phenyl)-6-(oxazol-2-yl)-1H- benzo[d]imidazol-1-yl)-5- methoxycyclohexyl)picolin- amide |
| 335 | 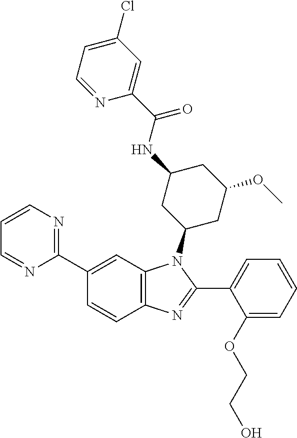 [SMILES: CO[C@H]1C[C@H](NC(=O)c2cc(Cl)ccn2)C[C@H](n2c(-c3ccccc3OCCO)nc3ccc(-c4ncccn4)cc32)C1] | 4-chloro-N-((1R,3S,5S)-3- (2-(2-(2-hydroxyethoxy)- phenyl)-6-(pyrimidin-2-yl)- 1H-benzo[d]imidazol-1-yl)- 5-methoxycyclohexyl)- picolinamide |
| 336 | 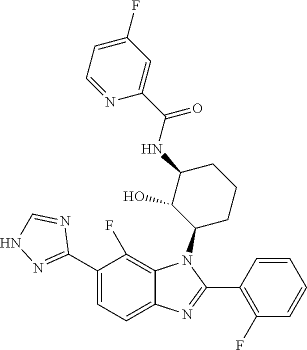 [SMILES: O=C(N[C@H]1CCC[C@@H](n2c(-c3ccccc3F)nc3ccc(-c4nc[nH]n4)c(F)c32)[C@@H]1O)c1cc(F)ccn1] | 4-fluoro-N-((1S,2R,3R)-3- (7-fluoro-2-(2-fluorophen- yl)-6-(1H-1,2,4-triazol-3- yl)-1H-benzo[d]imidazol-1- yl)-2-hydroxycyclohexyl)- picolinamide |
| 337 | 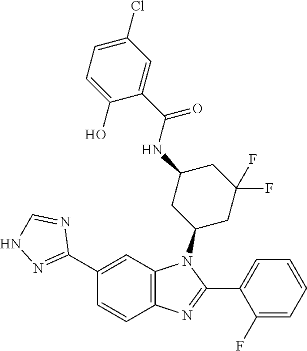 [SMILES: O=C(N[C@@H]1C[C@H](n2c(-c3ccccc3F)nc3ccc(-c4nc[nH]n4)cc32)CC(F)(F)C1)c1cc(Cl)ccc1O] | 5-chloro-N-((1R,5S)-3,3- difluoro-5-(2-(2-fluoro- phenyl)-6-(1H-1,2,4-triazol- 3-yl)-1H-benzo[d]imidazol- 1-yl)cyclohexyl)-2-hydroxy- benzamide |
| 338 | 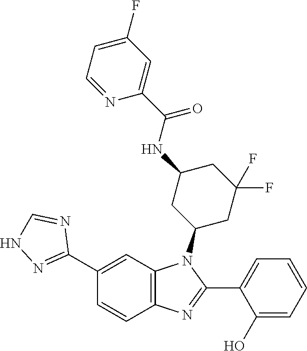 [SMILES: O=C(N[C@@H]1C[C@H](n2c(-c3ccccc3O)nc3ccc(-c4nc[nH]n4)cc32)CC(F)(F)C1)c1cc(F)ccn1] | N-((1R,5S)-3,3-difluoro-5- (2-(2-hydroxyphenyl)-6- (1H-1,2,4-triazol-3-yl)-1H- benzo[d]imidazol-1-yl)- cyclohexyl)-4-fluoropicolin- amide |
| 339 | 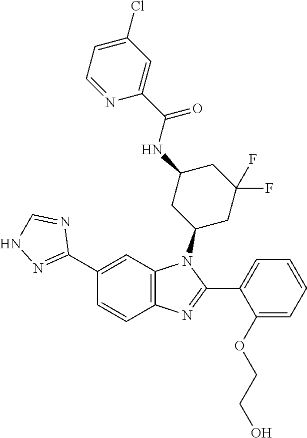 [SMILES: O=C(N[C@@H]1C[C@H](n2c(-c3ccccc3OCCO)nc3ccc(-c4nc[nH]n4)cc32)CC(F)(F)C1)c1cc(Cl)ccn1] | 4-chloro-N-((1R,5S)-3,3- difluoro-5-(2-(2-(2-hydroxy- ethoxy)phenyl)-6-(1H-1,2,4- triazol-3-yl)-1H-benzo[d] imidazol-1-yl)cyclohexyl)- picolinamide |
| 340 | 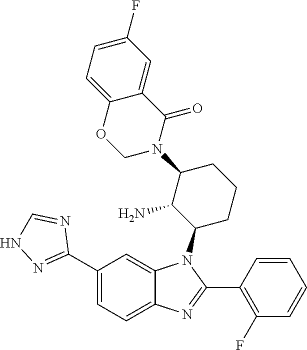 [SMILES: N[C@H]1[C@H](n2c(-c3ccccc3F)nc3ccc(-c4nc[nH]n4)cc32)CCC[C@@H]1N1COc2ccc(F)cc2C1=O] | 3-((1S,2S,3R)-2-amino-3-(2- (2-fluorophenyl)-6-(1H- 1,2,4-triazol-3-yl)-1H-benzo [d]imidazol-1-yl)cyclohex- yl)-6-fluoro-2,3-dihydro-4H- benzo[e][1,3]oxazin-4-one |
| 341 | 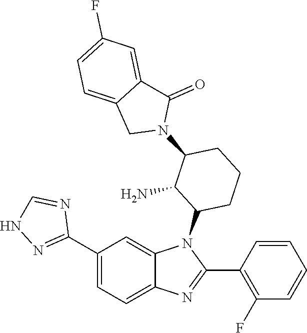 | 2-((1S,2S,3R)-2-amino-3- (2-(2-fluorophenyl)-6-(1H- 1,2,4-triazol-3-yl)-1H-benzo [d]imidazol-1-yl)cyclohex- yl)-6-fluoroisoindolin-1-one |
| 342 | 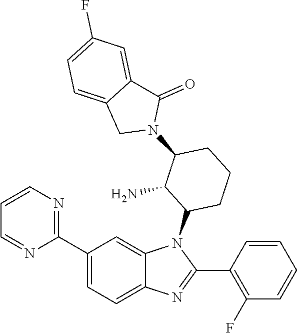 | 2-((1S,2S,3R)-2-amino-3-(2- (2-fluorophenyl)-6-(pyrimi- din-2-yl)-1H-benzo[d]imid- azol-1-yl)cyclohexyl)-6- fluoroisoindolin-1-one |
| 343 | 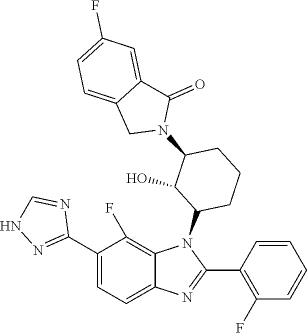 | 6-fluoro-2-((1S,2S,3R)-3- (7-fluoro-2-(2-fluorophen- yl)-6-(1H-1,2,4-triazol-3- yl)-1H-benzo[d]imidazol-1- yl)-2-hydroxycyclohexyl)- isoindolin-1-one |
| 344 | 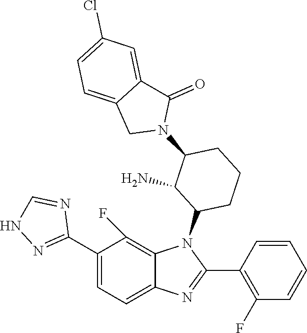 | 2-((1S,2S,3R)-2-amino-3- (7-fluoro-2-(2-fluorophen- yl)-6-(1H-1,2,4-triazol-3- yl)-1H-benzo[d]imidazol-1- yl)cyclohexyl)-6-chloroiso- indolin-1-one |
| 345 | 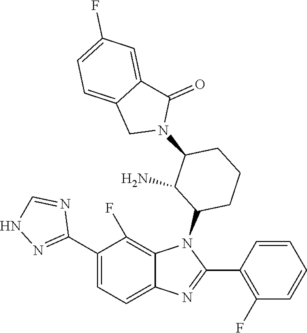 | 2-((1S,2S,3R)-2-amino-3- (7-fluoro-2-(2-fluorophen- yl)-6-(1H-1,2,4-triazol-3- yl)-1H-benzo[d]imidazol-1- yl)cyclohexyl)-6-fluoroiso- indolin-1-one |
| 346 | 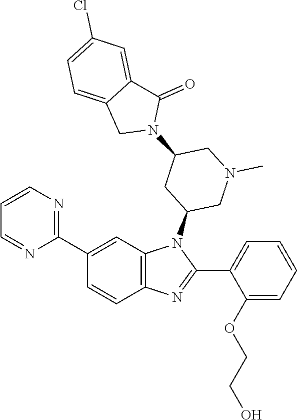 [SMILES: CN1C[C@H](N2Cc3ccc(Cl)cc3C2=O)C[C@H](n2c(-c3ccccc3OCCO)nc3ccc(-c4ncccn4)cc32)C1] | 6-chloro-2-((3R,5S)-5-(2- (2-(2-hydroxyethoxy)phen- yl)-6-(pyrimidin-2-yl)-1H- benzo[d]imidazol-1-yl)-1- methylpiperidin-3-yl)iso- indolin-1-one |
| 347 | 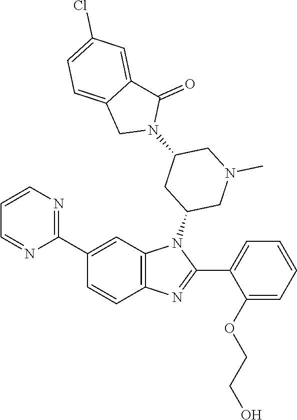 [SMILES: CN1C[C@@H](N2Cc3ccc(Cl)cc3C2=O)C[C@@H](n2c(-c3ccccc3OCCO)nc3ccc(-c4ncccn4)cc32)C1] | 6-chloro-2-((3S,5R)-5-(2- (2-(2-hydroxyethoxy)phen- yl)-6-(pyrimidin-2-yl)-1H- benzo[d]imidazol-1-yl)-1- methylpiperidin-3-yl)iso- indolin-1-one |

### Pharmaceutical Compositions

The disclosure also provides pharmaceutical compositions comprising compounds of the disclosure and a pharmaceutically acceptable carrier or excipient.

The methods of the present disclosure can be accomplished by administering compounds of the disclosure as the neat compound or as a pharmaceutical composition. Administration of a pharmaceutical composition, or neat compound of the disclosure, can be performed at any time period as determined by the attending physician. Typically, the pharmaceutical compositions contain no toxic, carcinogenic, or mutagenic compounds that would cause an adverse reaction when administered.

Pharmaceutical compositions include those wherein compounds of the disclosure are administered in an effective amount to achieve its intended purpose. The exact formulation, route of administration, and dosage is determined by an individual physician.

Compounds of the disclosure can be administered by any suitable route, e.g., by oral, buccal, inhalation, sublingual, rectal, vaginal, intracisternal or intrathecal through lumbar puncture, transurethral, nasal, percutaneous, i.e., transdermal, or parenteral (including intravenous, intramuscular, subcutaneous, intracoronary, intradermal, intramammary, intraperitoneal, intraarticular, intrathecal, retrobulbar, intrapulmonary injection and/or surgical implantation at a particular site) administration. Parenteral administration can be accomplished using a needle and syringe or using a high-pressure technique.

Compounds of the disclosure may be administered in admixture with pharmaceutical carriers selected with regard to the intended route of administration and standard pharmaceutical practice. Pharmaceutical compositions for use in accordance with the present disclosure are formulated in a conventional manner using one or more physiologically acceptable carriers comprising excipients and/or auxiliaries that facilitate processing of compounds of the disclosure.

Administration of the compounds or pharmaceutical compositions of the disclosure can be effected by any method that enables delivery of the compounds to the site of action. These methods include oral routes, intraduodenal routes, parenteral injection (including intravenous, intraarterial, subcutaneous, intramuscular, intravascular, intraperitoneal or infusion), topical (e.g., transdermal application), rectal administration, via local delivery by catheter or stent or through inhalation. Compounds can also be administered intraadiposally or intrathecally.

The amount of the compound administered will be dependent on the subject being treated, the severity of the disorder or condition, the rate of administration, the disposition of the compound and the discretion of the prescribing physician. The desired dose can be administered in a single dose, or as multiple doses administered at appropriate intervals, e.g., as one, two, three, four or more subdoses per day. In some embodiments, the compounds disclosed herein are effective over a wide dosage range. For example, in the treatment of adult humans, dosage forms containing from about 0.01 to 1000 mg of a compound disclosed herein per day are examples of dosage forms that may be used. The exact dosage will depend upon the route of administration, the form in which the compound is administered, the subject to be treated, the body weight of the subject to be treated, and the preference and experience of the attending physician. In some instances, dosage levels below the lower limit of the aforesaid range may be more than adequate, while in other cases still larger doses may be employed without causing any harmful side effect, e.g., by dividing such larger doses into several small doses for administration throughout the day.

In some embodiments, a compound of the disclosure is administered in a single dose.

Typically, such administration will be by a solid oral dosage form such as tablet or capsule. However, other routes may be used as appropriate. A single dose of a compound may also be used for treatment of an acute condition.

In some embodiments, a compound of the disclosure may be administered in multiple doses. Dosing may be about once, twice, three times, four times, five times, six times, or more than six times per day. In another embodiment, a compound described herein and another therapeutic agent are administered together about once per day to about 6 times per day. Administration of the compounds disclosed herein may continue as long as necessary. In some embodiments, a compound is administered chronically on an ongoing basis, e.g., for the treatment of chronic effects.

An effective amount of a compound of the disclosure may be administered in either single or multiple doses by any of the accepted modes of administration of therapeutic agents having similar utilities, including rectal, buccal, intranasal and transdermal routes, by intra-arterial injection, intravenously, intraperitoneally, parenterally, intramuscularly, subcutaneously, orally, topically, or as an inhalant.

The pharmaceutical composition may, for example, be in a form suitable for oral administration as a tablet, capsule, pill, powder, sustained release formulations, solution, or suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include one or more conventional pharmaceutical carriers or excipients and a compound disclosed herein as an active ingredient. In addition, it may include other medicinal or pharmaceutical agents, carriers, adjuvants, etc.

Exemplary parenteral administration forms include solutions or suspensions of the compound of the disclosure in sterile aqueous solutions, for example, aqueous propylene glycol or dextrose solutions. Such dosage forms can be suitably buffered, if desired.

Unless otherwise noted, the amounts of the compounds described herein are set forth on a free base basis. That is, the amounts indicate that amount of the compound administered, exclusive of, for example, solvent or counterions (such as in pharmaceutically acceptable salts).

Described below are non-limiting exemplary pharmaceutical compositions and methods for preparing the same.

### Pharmaceutical Compositions for Oral Administration.

In some embodiments, the disclosure provides a pharmaceutical composition for oral administration containing a compound of the disclosure and pharmaceutical excipients suitable for oral administration.

In some embodiments, the disclosure provides a solid pharmaceutical composition for oral administration containing: (i) an effective amount of a compound of the disclosure; optionally (ii) an effective amount of a second therapeutic agent; and (iii) a pharmaceutical excipient suitable for oral administration. In some embodiments, the pharmaceutical composition further contains: (iv) an effective amount of a third therapeutic agent.

In some embodiments, the pharmaceutical composition may be a pharmaceutical composition suitable for oral consumption. Pharmaceutical compositions containing a compound of the disclosure suitable for oral administration can be presented as discrete dosage forms, such as capsules, cachets, tablets, liquids, or aerosol sprays each containing a predetermined amount of an active ingredient as a powder or in granules, a solution or a suspension in an aqueous or non-aqueous liquid, an oil-in-water emulsion, or a water-in-oil liquid emulsion. Such dosage forms can be prepared by any of the methods of pharmacy, but all methods include the step of bringing the compound of the disclosure into association with the carrier, which constitutes one or more necessary ingredients. In general, the pharmaceutical compositions are prepared by uniformly and intimately admixing the compound of the disclosure with liquid carriers or finely divided solid carriers or both, and then, if necessary, shaping the product into the desired presentation. For example, a tablet can be prepared by compression or molding, optionally with one or more accessory ingredients. Compressed tablets can be prepared by compressing in a suitable machine the active ingredient in a free-flowing form such as powder or granules, optionally mixed with an excipient such as, but not limited to, a binder, a lubricant, an inert diluent, and/or a surfactant or dispersing agent. Molded tablets can be made by molding in a suitable machine a mixture of the powdered compound moistened with an inert liquid diluent.

This disclosure further encompasses anhydrous pharmaceutical compositions and dosage forms comprising a compound of the disclosure, since water can facilitate the degradation of some compounds. For example, water may be added (e.g., 5%) in the pharmaceutical arts as a means of simulating long-term storage in order to determine characteristics such as shelf-life or the stability of formulations over time. Anhydrous pharmaceutical compositions and dosage forms containing a compound of the disclosure can be prepared using anhydrous or low moisture containing ingredients and low moisture or low humidity conditions. Pharmaceutical compositions and dosage forms containing a compound of the disclosure which contain lactose can be made anhydrous if substantial contact with moisture and/or humidity during manufacturing, packaging, and/or storage is expected. An anhydrous pharmaceutical composition may be prepared and stored such that its anhydrous nature is maintained. Accordingly, anhydrous compositions may be packaged using materials known to prevent exposure to water such that they can be included in suitable formulary kits. Examples of suitable packaging include, but are not limited to, hermetically sealed foils, plastic or the like, unit dose containers, blister packs, and strip packs.

The compound of the disclosure can be combined in an intimate admixture with a pharmaceutical carrier according to conventional pharmaceutical compounding techniques. The carrier can take a wide variety of forms depending on the form of preparation desired for administration. In preparing the pharmaceutical compositions for an oral dosage form, any of the usual pharmaceutical media can be employed as carriers, such as, for example, water, glycols, oils, alcohols, flavoring agents, preservatives, coloring agents, and the like in the case of oral liquid preparations (such as suspensions, solutions, and elixirs) or aerosols; or carriers such as starches, sugars, micro-crystalline cellulose, diluents, granulating agents, lubricants, binders, and disintegrating agents can be used in the case of oral solid preparations, in some embodiments without employing the use of lactose. For example, suitable carriers include powders, capsules, and tablets, with the solid oral preparations. If desired, tablets can be coated by standard aqueous or nonaqueous techniques.

Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, colloidal silicon dioxide, and mixtures thereof.

Examples of suitable fillers for use in the pharmaceutical compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

Disintegrates may be used in the pharmaceutical compositions and dosage forms disclosed herein to provide tablets that disintegrate when exposed to an aqueous environment. Too much of a disintegrate may produce tablets which may disintegrate in the bottle. Too little may be insufficient for disintegration to occur and may thus alter the rate and extent of release of the active ingredient(s) from the dosage form. Thus, a sufficient amount of disintegrate that is neither too little nor too much to detrimentally alter the release of the active ingredient(s) may be used to form the dosage forms of the compounds disclosed herein. The amount of disintegrate used may vary based upon the type of formulation and mode of administration, and may be readily discernible to those of ordinary skill in the art. About 0.5 to about 15 weight percent of disintegrate, or about 1 to about 5 weight percent of disintegrate, may be used in the pharmaceutical composition. Disintegrates that can be used to form pharmaceutical compositions and dosage forms of the disclosure include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

Lubricants which can be used to form pharmaceutical compositions and dosage forms of the disclosure include, but are not limited to, calcium stearate, magnesium stearate, sodium stearyl fumarate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel, a coagulated aerosol of synthetic silica, and mixtures thereof. A lubricant can optionally be added, in an amount of less than about 2 weight percent of the pharmaceutical composition.

When aqueous suspensions and/or elixirs are desired for oral administration, the active ingredient therein may be combined with various sweetening or flavoring agents, coloring matter or dyes and, if so desired, emulsifying and/or suspending agents, together with such diluents as water, ethanol, propylene glycol, glycerin and various combinations thereof.

The tablets can be uncoated or coated by known techniques to delay disintegration and absorption in the gastrointestinal tract and thereby provide a sustained action over a longer period. For example, a time delay material such as glyceryl monostearate or glyceryl distearate can be employed. Formulations for oral use can also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Surfactants which can be used to form pharmaceutical compositions and dosage forms of the disclosure include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed.

A suitable hydrophilic surfactant may generally have an HLB value of at least 10, while suitable lipophilic surfactants may generally have an HLB value of or less than about 10. An empirical parameter used to characterize the relative hydrophilicity and hydrophobicity of non-ionic amphiphilic compounds is the hydrophilic-lipophilic balance (“HLB” value). Surfactants with lower HLB values are more lipophilic or hydrophobic, and have greater solubility in oils, while surfactants with higher HLB values are more hydrophilic, and have greater solubility in aqueous solutions.

Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof; polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG1000 succinate, PEG-24 cholesterol, polyglyceryl-10-oleate, Tween 40, Tween 60, sucrose monostearate, sucrose mono laurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the pharmaceutical composition may include a solubilizer to ensure good solubilization and/or dissolution of the compound of the disclosure and to minimize precipitation of the compound of the disclosure. This can be important for compositions for non-oral use, e.g., compositions for injection. A solubilizer may also be added to increase the solubility of the hydrophilic drug and/or other components, such as surfactants, or to maintain the pharmaceutical composition as a stable or homogeneous solution or dispersion.

Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; polyethylene glycol 660 12-hydroxystearate, amides and other nitrogen-containing compounds such as 2-pyrrolidone, 2-piperidone, F-caprolactam, N-alkylpyrrolidone, N-hydroxyalkylpyrrolidone, N-alkylpiperidone, N-alkylcaprolactam, dimethylacetamide and polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, F-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, glycofurol, transcutol, propylene glycol, and dimethyl isosorbide. Particularly preferred solubilizers include sorbitol, glycerol, triacetin, ethyl alcohol, PEG having a molecular weight between 100 and 8000 g/mole, glycofurol and propylene glycol.

The amount of solubilizer that can be included is not particularly limited. The amount of a given solubilizer may be limited to a bioacceptable amount, which may be readily determined by one of skill in the art. In some circumstances, it may be advantageous to include amounts of solubilizers far in excess of bioacceptable amounts, for example to maximize the concentration of the drug, with excess solubilizer removed prior to providing the pharmaceutical composition to a subject using conventional techniques, such as distillation or evaporation. Thus, if present, the solubilizer can be in a weight ratio of less than about 10%, less than about 25%, less than about 50%, about 100%, or up to less than about 200% by weight, based on the combined weight of the drug and other excipients. If desired, very small amounts of solubilizer may also be used, such as less than about 5%, less than about 2%, less than about 1% or even less. Typically, the solubilizer may be present in an amount of less than about 1% to about 100%, more typically less than about 5% to less than about 25% by weight.

The pharmaceutical composition can further include one or more pharmaceutically acceptable additives and excipients. Such additives and excipients include, without limitation, detackifiers, anti-foaming agents, buffering agents, polymers, antioxidants, preservatives, chelating agents, viscomodulators, tonicifiers, flavorants, colorants, odorants, opacifiers, suspending agents, binders, fillers, plasticizers, lubricants, and mixtures thereof.

### Pharmaceutical Compositions for Injection.

In some embodiments, the disclosure provides a pharmaceutical composition for injection containing a compound described herein and pharmaceutical excipients suitable for injection. Components and amounts of agents in the pharmaceutical compositions are as described herein.

The forms in which the pharmaceutical compositions of the disclosure may be incorporated for administration by injection include aqueous or oil suspensions or emulsions. Such compositions may comprise sesame oil, corn oil, cottonseed oil, peanut oil, elixirs containing mannitol or dextrose, sterile water, and similar pharmaceutical vehicles.

Aqueous solutions in saline are also conventionally used for injection. Ethanol, glycerol, propylene glycol, liquid polyethylene glycol, and the like (and suitable mixtures thereof), cyclodextrin derivatives, and vegetable oils may also be employed. The proper fluidity can be maintained, for example, by the use of a coating, such as lecithin, for the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like.

Sterile injectable solutions are prepared by incorporating the compound of the disclosure in the required amount in the appropriate solvent with various other ingredients as enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, certain desirable methods of preparation are vacuum-drying and freeze-drying techniques which yield a powder of the active ingredient plus any additional desired ingredient from a previously sterile-filtered solution thereof.

### Other Pharmaceutical Compositions.

Pharmaceutical compositions may also be prepared from compositions described herein and one or more pharmaceutically acceptable excipients suitable for topical, sublingual, buccal, rectal, intraosseous, intraocular, intranasal, epidural, or intraspinal administration. Preparations for such pharmaceutical compositions are well-known in the art. See, e.g., Anderson, Philip O.; Knoben, James E.; Troutman, William G, eds., Handbook of Clinical Drug Data, Tenth Edition, McGraw-Hill, 2002; Pratt and Taylor, eds., Principles of Drug Action, Third Edition, Churchill Livingston, New York, 1990; Katzung, ed., Basic and Clinical Pharmacology, Ninth Edition, McGraw Hill, 2004; Goodman and Gilman, eds., The Pharmacological Basis of Therapeutics, Tenth Edition, McGraw Hill, 2001; Remington's Pharmaceutical Sciences, 20th Ed., Lippincott Williams & Wilkins, 2000; Martindale, The Extra Pharmacopoeia, Thirty-Second Edition (The Pharmaceutical Press, London, 1999); all of which are incorporated by reference herein in their entirety.

### Methods of Use

The methods disclosed herein typically comprise administering to a subject a therapeutically effective amount of a compound or pharmaceutical composition disclosed herein. The therapeutically effective amount of a compound of the disclosure may vary depending upon the intended application (in vitro or in vivo), the subject and disease condition being treated, e.g., the weight and age of the subject, the severity of the disease condition, the manner of administration and the like. In some embodiments, a therapeutically effective amount refers to a dose that will induce a particular response in target cells. The specific dose will vary depending on the particular compounds chosen, the dosing regimen to be followed, whether it is administered in combination with other compounds, timing of administration, the tissue to which it is administered, and the physical delivery system in which it is carried.

In treatment methods of the disclosure, a therapeutically effective amount of a compound of the disclosure (or a pharmaceutically acceptable salt thereof) is administered to a subject suffering from or diagnosed as having such a disease or disorder. An example of a dose is in the range of from about 0.001 to about 100 mg of compound per kg of subject's body weight per day, in single or divided dosage units (e.g., BID, TID, QID). For a 70-kg human, a suitable dosage amount is from about 0.05 to about 7 g/day.

In addition, the compounds of the disclosure may be used in combination with additional active ingredients in the treatment of the diseases or disorders disclosed herein. The additional active ingredients may be coadministered separately with a compound of the disclosure or included with such an agent in a pharmaceutical composition according to the disclosure. The combination may serve to increase efficacy (e.g., by including in the combination a compound potentiating the potency or effectiveness of an active agent according to the disclosure), decrease one or more side effects, or decrease the required dose of the active agent according to the disclosure.

In some embodiments, the disclosure is directed to a method of treating a BCAA-catabolism-related disease comprising administering a compound or pharmaceutical composition of the disclosure.

In some embodiments, the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency (also known as short/branched chain acyl-CoA dehydrogenase deficiency), beta-ketothiolase deficiency (BKT), hydroxy-3-methylglutaconyl-CoA lysase (HMGCL) deficiency, 3-methylglutaconyl-CoA-hydratase deficiency (MGA), 3-methylcrotonyl-CoA (3-MCC) deficiency, glutaric aciduria type 1 (GA1), 3-hydroxy-3-methylglutaryl-CoA lyase deficiency (MGA), 3-methylglutaconic acidemia, D-2 hydroxyglutaric aciduria (D2-HGA), isobutyryl-CoA dehydrogenase deficiency, 3-hydroxyisobutyric aciduria (ICBD), L-2-hydroxy-glutaricaciduria (L2HGA), malonyl-CoA decarboxylase deficiency aka malonic acidemia (MA), multiple carboxylase deficiency (MCD, holocarboxylase synthetase), or 2-methyl-3-hydroxybutyryl-CoA dehydrogenase (MHBD) deficiency.

In some embodiments, the BCAA-catabolism-related disease is an organic acidemia.

In some embodiments, the BCAA-catabolism-related disease is methylmalonic acidemia (MMA), propionic acidemia (PA), isovaleric acidemia (IVA), or maple syrup urine disease.

In some embodiments, the BCAA-catabolism-related disease is methylmalonic acidemia (MMA).

In some embodiments, the BCAA-catabolism-related disease is propionic acidemia (PA).

In some embodiments, the BCAA-catabolism-related disease is isovaleric acidemia (IVA).

In some embodiments, the BCAA-catabolism-related disease is maple syrup urine disease.

In some embodiments, the methods of the disclosure reduce and/or ameliorate the symptoms of the BCAA-catabolism-related disease in the subject.

In some embodiments, the disclosure provides methods of reducing toxic BCAA metabolites in one or more bodily fluids of the subject such as the blood or urine of a subject, comprising administering a therapeutically effective amount of a BCAT2 inhibitor of the disclosure. As used here, the term “reducing” means that the BCAA metabolite levels in the subject's bodily fluids are lower after being administered a therapeutically effective amount of a BCAT2 inhibitor of the disclosure than the level prior to administration. Methods of measuring the levels of BCAA metabolites in blood and urine and normal ranges of those metabolites in such bodily fluids are known in the art.

In some embodiments, the methods cause a decrease in toxic branched chain amino acid (BCAA) metabolite levels.

In some embodiments, the toxic metabolite is ammonia, propionic acid, 2-methylcitric acid, 3-hydroxypropionic acid, propionyl-CoA, methylmalonic acid, methylmalonyl-CoA or isovaleric acid. In some embodiments, the toxic metabolite is propionic acid, 2-methylcitric acid, 3-hydroxypropionic acid, propionyl-CoA, methylmalonic acid, methylmalonyl-CoA or isovaleric acid.

In some embodiments, the toxic metabolites are allosteric enzyme inhibitors.

In some embodiments, the methods of the disclosure cause an increase in BCAA levels in the blood of a subject. As used here, the term “increase” means that the BCAA level in the subject's blood is greater after being administered a therapeutically effective amount of a BCAT2 inhibitor of the disclosure than the level prior to the administration. Methods of measuring the blood levels of BCAAs are known in the art.

In some embodiments, the BCAA is one or more of leucine, isoleucine, or valine.

In some embodiments, the methods of the disclosure modulate metabolic flux through a BCAA-catabolism pathway. As used herein, “metabolic flux” refers to the rate of passage of molecules through a metabolic pathway. The term modulate means to change, i.e., to increase or decrease. Methods of measuring metabolic flux are known in the art. Thus, in such embodiments, the metabolic flux through a BCAA-catabolism pathway is different before and after administering a BCAT2 inhibitor of the disclosure.

In some embodiments, the methods of the disclosure decrease metabolic flux through a BCAA-catabolism pathway.

In some embodiments, the methods of the disclosure decrease BCAT2-mediated metabolic flux through a BCAA-catabolism pathway.

In some embodiments, the reduction of BCAT2-mediated flux occurs by reduction or inhibition of leucine catabolism. In some embodiments, leucine catabolism is reduced or inhibited at one or more enzymes in the BCAA-catabolism pathway, wherein the enzyme is branched chain α-ketoacid dehydrogenase complex (BCKDH complex), isovaleryl CoA dehydrogenase (IVD), 3-methylcrotonyl-CoA carboxylase (3MCC), 3-methylglutaconyl-CoA hydratase (3MGA), 3-hydroxy-3-methylglutaryl-CoA lyase (HMGL), acetyl-CoA carboxylase (AC), or malonyl-CoA decarboxylase (MA).

In some embodiments, the reduction of BCAT2-mediated flux occurs by reduction or inhibition of isoleucine catabolism. In some embodiments, isoleucine catabolism is reduced or inhibited at one or more enzymes in the BCAA-catabolism pathway, wherein the enzyme is BCKDH complex, methylbutyryl CoA dehydrogenase (SBCAD), mitochondrial short-chain enoyl-CoA hydratase, 2-methyl-3-hydroxyisobutyryl-CoA dehydrogenase (MHBD), acetoacetyl-CoA thiolase/beta-ketothiolase (T2/ACAT1), AC, MA, propionyl-CoA carboxylase (PCC), or methylmalonyl-CoA mutase (MUT), or succinyl-CoA ligase (SUCLA).

In some embodiments, the reduction of BCAT2-mediated flux occurs by reduction or inhibition of valine catabolism. In some embodiments, valine catabolism is reduced or inhibited at one or more enzymes in the BCAA-catabolism pathway, wherein the enzyme is BCKDH complex, isobutyryl-CoA dehydrogenase (IBDH), mitochondrial short-chain enoyl-CoA hydratase, 3-hydroxyisobutyryl-CoA deacylase (hydrolase) (HIBDA), 3-hydroxyisobutyrate dehydrogenase (HIBDH), methylmalonic semialdehyde dehydrogenase (MMSDH), PCC, MUT, or SUCLA.

In some embodiments, the disclosure provides methods of reducing a toxic load burden in cells of a subject having a BCAA-catabolism-related disease, comprising administering a therapeutically effective amount of a BCAT2 inhibitor of the disclosure.

The present disclosure provides compounds of the disclosure as BCAT2 inhibitors for the treatment of diseases and conditions wherein inhibition of BCAT2 has a beneficial effect. Compounds of the disclosure typically inhibit BCAT2 (IC₅₀) in a concentration less than 20 μM, e.g., less than 10 μM, less than 5 μM, less than 3 μM, and less than 2 μM, less than about 1 μM, less than about 0.5 μM, less than about 0.1 μM, less than about 0.05 μM, or less than about 0.01 μM. In some embodiments, the present disclosure relates to a method of treating a subject suffering from a disease or condition wherein inhibition of BCAT2 provides a benefit comprising administering a therapeutically effective amount of a compound of the disclosure to a subject in need thereof.

In some embodiments, the disclosure is directed to methods of reducing plasma methylmalonic acid in a subject having an elevated methylmalonic acid level comprising administering to the subject a therapeutically effective amount of a compound of the disclosure. As used herein, “reducing” means that the plasma methylmalonic acid level in the subject is lower after administering the compound of the disclosure than before administering the compound of the disclosure. Methods of measuring plasma methylmalonic acid levels in plasma are known in the art. See, e.g., Manoli I, et al. Isolated Methylmalonic Acidemia. 2005 Aug. 16 [Updated 2016 Dec. 1]. In: Adam M P, Ardinger H H, Pagon R A, et al., editors. GeneReviews® [Internet]. Seattle (WA): University of Washington, Seattle; 1993-2021. As used herein, “elevated methylmalonic acid level” refers to a plasma methylmalonic acid level that is higher than the normal level expected for a given subject. See Anna-Kaisa Niemi, et al., Treatment of Methylmalonic Acidemia by Liver or Combined Liver-Kidney Transplantation, The Journal of Pediatrics, Volume 166, Issue 6, 2015, 1455-1461.e1. Normal blood level of methylmalonic acid in humans is <0.27 μmol/L. Elevated blood levels of methylmalonic acid levels in humans range from 5-1000 μmol/L, depending in part on the enzyme deficiency that is causing the patient's MMA.

In some embodiments, the disclosure is directed to methods of reducing blood ammonia in a subject having an elevated ammonia level (hyperammonemia) comprising administering to the subject a therapeutically effective amount of a compound of the disclosure. As used here, “reducing” means that the blood ammonia level in the subject is lower after administering the compound of the disclosure than before administering the compound of the disclosure. Normal blood level of ammonia in humans generally range from 11-32 μmol/L. A blood ammonia level above 75 μmol/L is associated with symptomatic decompensation and a blood ammonia level above 200 μmol/L is associated with impaired vigilance. See Nicola Longo et al., Hyperammonaemia in classic organic acid: a review of the literature and two case histories, Orphanet Journal of Rare Diseases, Volume 13, Issue 1, 2018, 219.

In some embodiments, the disclosure is directed to methods of reducing blood 2-methylcitrate in a subject having an elevated 2-methylcitrate level comprising administering to the subject a therapeutically effective amount of a compound of the disclosure. As used here, “reducing” means that the blood 2-methylcitrate level in the subject is lower after administering the compound of the disclosure than before administering the compound of the disclosure. Normal blood level of 2-methylcitrate in humans is ≤0.63 μmol/L, whereas blood 2-methylcitrate in MMA and PA patients range between 1.0-12.0 μmol/L according to one report. Osama Y. Al-Dirbashi et al., Assessment of methylcitrate and methylcitrate to citrate ratio in dried blood spots as biomarkers for inborn errors of propionate metabolism, Volume 9, 2019, 123666.

In some embodiments, the methods of the disclosure are performed in combination with other treatment strategies.

In some embodiments, the other treatment strategies are aimed at reducing the levels of toxic metabolites.

In some embodiments, the treatment strategies aimed at reducing the levels of toxic metabolites are directed to reducing the supply of BCAAs. Such strategies include isoleucine, valine, threonine and methionine restriction as part of a protein-restricted diet (with or without amino acid supplementation), eating a high caloric diet during illness, administering antibiotics, administering laxatives, or reducing odd-chain fatty acids and cholesterol.

In some embodiments, the treatment strategies aimed at reducing the levels of toxic metabolites are directed to increasing enzyme activity. Such embodiments include administering hydroxycobalamin, solid organ transplantation (liver or combined liver-kidney transplantation), gene therapy, promotion of premature stop codon read-through, messenger RNA therapy, pharmacological stabilization of enzyme activity, enzyme replacement therapy, or hepatocyte transplantation.

In some embodiments, the treatment strategies aimed at reducing the levels of toxic metabolites are directed to increasing disposal of toxic metabolites. Such embodiments include administering a carbamoyl phosphate synthetase 1 (CPS 1) activator, administering N-carbamylglutamate (CARBAGLU®), administering sodium benzoate, extracorporeal detoxification, administering L-carnitine, or administering sodium bicarbonate.

In some embodiments, the other treatment strategies are aimed at preventing or treating mitochondrial energetic failure and increase of reactive oxygen species (ROS) formation.

In some embodiments, other treatment strategies aimed at preventing or treating mitochondrial energetic failure and increase of reactive oxygen species (ROS) formation are directed to effecting energetic supply. Such embodiments include administering succinate, citric acid, or creatine.

In some embodiments, other treatment strategies aimed at preventing or treating mitochondrial energetic failure and increase of reactive oxygen species (ROS) formation are directed to cofactor supplementation. Such embodiments include administering pyridoxine (vitamin B6) or administering thiamine (vitamin B1).

In some embodiments, other treatment strategies aimed at preventing or treating mitochondrial energetic failure and increase of reactive oxygen species (ROS) formation are directed to anti-oxidant supplementation. Such embodiments include administering coenzyme Q10, ascorbic acid (vitamin C), alpha-tocopherol (vitamin E), CB agonist WIN, S-allylcysteine, gluthathione, GM1 ganglioside, melatonin, mitoQ, resveratrol, tiron, or trolox.

In some embodiments, the other treatment strategies that are aimed at preventing or treating mitochondrial energetic failure and increase of reactive oxygen species (ROS) formation are directed to electron transfer mediator supplementation. Such embodiments include administering methylene blue.

In some embodiments, the other treatment strategies that are aimed at preventing or treating mitochondrial energetic failure and increase of reactive oxygen species (ROS) formation are directed to administering NMDA receptor antagonists. Such embodiments include administering celecoxib, fish oil, kynurenic acid, or MK-801.

In some embodiments, the other treatment strategies include administering an inhibitor of an amino acid transporter, optionally with a SLC6A19 inhibitor. In further embodiments, the amino acid transporter is B⁰AT1 (also referred to as SLC6A19), and the inhibitor is a SLC6A19 inhibitor. Examples of SLC6A19 inhibitors include nimesulide, benztropine, NSC63912, NSC22789, cinromide, CB3, E62, JNT-517, and the like.

In some embodiments, performing the methods of the disclosure in combination with the other treatment strategies results in levels of branched chain amino acids that are reduced compared to treatment without performance of the other treatment strategy.

### Animal Models

The present disclosure also provides in vivo models for the study of disorders characterized by defects in the metabolism of branched-chain amino acids (BCAAs), e.g., leucine, isoleucine and valine. These disorders include BCAT2 deficiency, methylmalonic acidemia, and propionic acidemia.

The first step in branched-chain amino acid (BCAA) catabolism is catalyzed by the two BCAA transferase isoenzymes, cytoplasmic branched-chain amino acid transferase (BCAT) 1, and mitochondrial BCAT2. BCAT2 deficiency is an inborn error of BCAA catabolism. BCAT2 deficiency has a recognizable biochemical profile with raised plasma BCAAs, low-normal branched-chain keto acids (BCKAs), and undetectable L-allo-isoleucine. The biochemical characteristics of BCAT2 deficiency may be amenable to protein-restricted diet, and early treatment may improve outcome in affected individuals. See Knerr et al., J Inherit Metab Dis. 42(5): 809-817 (2019).

Methylmalonic acidemia refers to a group of inborn errors of metabolism associated with elevated methylmalonic acid (MMA) concentration in the blood and urine that result from the failure to isomerize (convert) methylmalonyl-coenzyme A (CoA) into succinyl-CoA during propionyl-CoA metabolism in the mitochondrial matrix. Two main forms of the disease have been identified, isolated methylmalonic acidemia and combined methylmalonic acidemia and homocystinuria. Isolated methylmalonic acidemia is due to defects of methylmalonyl-CoA mutase or the synthesis of the MUT coenzyme adenosylcobalamin (AdoCbl), while combined methylmalonic acidemia and homocystinuria is characterized by elevated plasma homocysteine and decreased levels of the coenzymes adenosylcobalamin (AdoCbl) and methylcobalamin (MeCbl). Methylmalonic acidemia is caused by complete or partial deficiency of the enzyme methylmalonyl-CoA mutase, a defect in the transport or synthesis of its cofactor, 5-deoxy-adenosyl-cobalamin (cblA, cblB, or cblD-MMA), or deficiency of the enzyme methylmalonyl-CoA epimerase. Methylmalonic acidemia has a wide clinical spectrum, ranging from a benign condition to fetal neonatal disease. Affected children usually exhibit anorexia, failure to thrive, hypotonia, developmental delay, progressive renal failure, functional immune impairment, optic nerve atrophy, and hematologic abnormalities. See Zhou et al., Intractable Rare Dis Res. 7(2): 73-78 (2018).

Propionic acidemia (PA) is caused by deficiency of the mitochondrial multimeric enzyme propionyl-CoA carboxylase (PCC) that catalyzes the conversion of propionyl-CoA to D-methylmalonyl-CoA. The enzyme is composed of α- and β-subunits encoded by their respective genes, PCCA and PCCB. PA is caused by deficiency in either the a or R subunits of propionyl-CoA carboxylase. Deficient activity of propionyl-CoA carboxylase results in accumulation of propionic acid and propionyl-CoA related metabolites, which can be detected biochemically. This disease causes metabolic acidosis, ketosis, vomiting, lethargy, cognitive reductions, and death. See Barry, Expert Review of Precision Medicine and Drug Development, 4:4, 229-237 (2019).

Transgenic mice for use as animal models of these disorders are described below.

### BCAT2 Knockout Mice as an Animal Model of BCAT2 Deficiency

BCAT2 catalyzes the first step in BCAA metabolism, which is transfer of the α-amino group of a BCAA to α-ketoglutarate to form glutamate and the three respective branched chain α-keto acids.

In certain aspects, the disclosure relates to a mouse (Mus musculus) comprising one or more genomic mutations in an endogenous BCAT2 gene that results in a knockout of the endogenous BCAT2 gene. In some embodiments, the mouse is an animal model for Branched Chain Amino Acid Transaminase 2 (BCAT2) deficiency.

In some embodiments, the one or more mutations are in exon 3 of the BCAT2 gene. In some embodiments, the one or more mutations comprise or consist of a deletion in exon 3 of the BCAT2 gene. In some embodiments, the one or more mutations comprise or consist of a deletion of only one nucleotide in exon 3 of the BCAT2 gene. Exon 3 of the BCAT2 gene corresponds to positions 127-330 of SEQ ID NO: 1. In some embodiments, the one or more mutations in exon 3 of the BCAT2 gene comprise or consist of a deletion of adenine (A) at position 245 of SEQ ID NO: 1.

In some embodiments, the one or more mutations result in a premature stop codon in the BCAT2 gene. In some embodiments, the premature stop codon is in exon 4 of the BCAT2 gene. Exon 4 of the BCAT2 gene corresponds to positions 331-441 of SEQ ID NO: 1. In some embodiments, the premature stop codon in exon 4 of the BCAT2 gene is a TGA stop codon at positions 344-346 of SEQ ID NO: 1.

In some embodiments, the mouse is homozygous for the knockout of the endogenous BCAT2 gene. These mice are designated as BCAT2^{KO/KO} mice herein. In some embodiments, the mouse is heterozygous for the knockout of the endogenous BCAT2 gene. These mice are designated as BCAT2^{KO/WT} herein.

In some embodiments, the mouse is generated by CRISPR/Cas9-mediated homology-directed repair (HDR) that introduces the one or more genomic mutations into the endogenous BCAT2 gene. In some embodiments, a single guide RNA (sgRNA) encoded by the polynucleotide sequence of SEQ ID NO: 8 and an sgRNA encoded by the polynucleotide sequence of SEQ ID NO: 9 are used for the CRISPR/Cas9-mediated HDR.

In some embodiments, the mouse comprising a knockout of one or more BCAT2 genes has increased plasma isoleucine levels relative to a mouse that does not comprise a knockout of an endogenous BCAT2 gene. In some embodiments, BCAT2^{KO/KO} mice have at least a 2-fold, 3-fold, 4-fold, 5-fold, 10-fold, 20-fold, 50-fold or 100-fold increase in plasma isoleucine levels relative to a mouse that does not comprise a knockout of an endogenous BCAT2 gene. The term “2-fold increase” as used herein refers to a doubling of a value relative to a standard, e.g., an increase in plasma isoleucine levels from 10 μM to 20 μM. The term “3-fold increase” as used herein refers to a tripling of a value relative to a standard, etc. In some embodiments, BCAT2^{KO/KO} mice have at least a 10-fold increase in plasma isoleucine levels relative to a mouse that does not comprise a knockout of an endogenous BCAT2 gene.

In some embodiments, the mouse comprising a knockout of one or more BCAT2 genes has reduced plasma levels of 3-HIB (a downstream metabolite of valine breakdown) relative to a mouse that does not comprise a knockout of an endogenous BCAT2 gene. In some embodiments, BCAT2^{KO/KO} mice have at least a 20%, 30%, 40%, 50%, 60%, 70%, 80% or 90% reduction in plasma 3-HIB levels relative to a mouse that does not comprise a knockout of an endogenous BCAT2 gene. In some embodiments, BCAT2^{KO/KO} mice have at least a 50% reduction in plasma 3-HIB levels relative to a mouse that does not comprise a knockout of an endogenous BCAT2 gene.

### MMA-CoA Mutase M698K/M698K, M698K/KO, and Combination with BCAT2 KO

Methylmalonyl-CoA mutase is a nuclear-encoded mitochondrial enzyme that catalyzes the conversion of L-methylmalonyl-CoA to succinyl-CoA, which then enters the citric acid cycle. L-Methylmalonyl-CoA is predominantly derived from the catabolism of branched chain amino acids in the diet, odd chain fatty acids via propionyl-CoA, and propionate synthesized from gut flora. See Peters, et al., J Biol. Chem. 278(52): 52909-52913 (2003).

In certain aspects, the disclosure relates to a mouse (Mus musculus) comprising one or more genomic mutations in at least one endogenous methylmalonyl Coenzyme A (MMA-CoA) mutase gene that results in an M698K substitution relative to the amino acid sequence of SEQ ID NO: 4 in a MMA-CoA protein encoded by the gene. In some embodiments, the mouse is an animal model for methylmalonic academia. Any one or more genomic mutations that result in an M698K mutation in MMA-CoA mutase may be used. In some embodiments, the one or more genomic mutations consist of or comprise a T2228A substitution relative to the nucleic acid sequence of SEQ ID NO: 3. Other genomic mutations that result in an M698K substitution in MMA-CoA mutase may also be used. For example, in some embodiments, the T2228A substitution relative to the nucleic acid sequence of SEQ ID NO: 3 may be combined with a G2229A substitution relative to the nucleic acid sequence of SEQ ID NO: 3. The combination of the T2228A and G2229 substitutions converts the ATG codon (encoding methionine) at positions 2227-2229 of SEQ ID NO: 3 to an AAA codon (encoding lysine).

In some embodiments, the mouse is homozygous for the one or more genomic mutations in the endogenous MMA-CoA mutase gene that results in the M698K substitution. These homozygous mice are designated as MUT^{M698K/M68K} mice herein. In some embodiments, the mouse is heterozygous for the one or more genomic mutations in the endogenous MMA-CoA mutase gene that results in the M698K substitution. These heterozygous mice are designated as Mut^{M698K/WT} herein. In some embodiments, the mouse comprises the one or more genomic mutations that results in the M698K substitution at a first locus of an endogenous MMA-CoA mutase gene, and further comprises one or more genomic mutations that result in a knockout of an endogenous MMA-CoA mutase gene at a second locus of an endogenous MMA-CoA mutase gene. These mice are designated as MUT^{M698K/KO} mice herein.

In some embodiments, the mice comprising the M698K substitution in the MMA-CoA mutase gene are generated by CRISPR/Cas9-mediated homology-directed repair (HDR) that introduces the one or more genomic mutations into the endogenous MMA-CoA mutase gene that results in the M698K substitution. In some embodiments, a single guide RNA (sgRNA) encoded by the polynucleotide sequence of SEQ ID NO: 19 and an sgRNA encoded by the polynucleotide sequence of SEQ ID NO: 20 are used for the CRISPR/Cas9-mediated HDR to generate the M698K substitution. In some embodiments, the CRISPR/Cas9-mediated HDR utilizes a donor DNA having the polynucleotide sequence of SEQ ID NO: 21 to generate the M698K substitution.

In some embodiments, the one or more genomic mutations that result in a knock out of the endogenous MMA-CoA mutase gene is in exon 2 of the endogenous MMA-CoA mutase gene. Exon 2 of the MMA-CoA mutase gene corresponds to positions 96 to 514 of SEQ ID NO: 3. In some embodiments, the one or more genomic mutations that result in a knock out of the endogenous MMA-CoA mutase gene introduce a premature stop codon into the endogenous MMA-CoA mutase gene. For example, in some embodiments, in the MMA-CoA mutase knockout mice the guanine (g) at position 311 of SEQ ID NO: 3 is changed to adenine (A), resulting in the codon encoding tryptophan (TGG) at positions 310-312 of SEQ ID NO: 3 being converted to a stop codon (TAG). Accordingly, in some embodiments, the one or more genomic mutations that result in a knock out of the endogenous MMA-CoA mutase gene comprise or consist of a G311A substitution relative to the nucleic acid sequence of SEQ ID NO: 3. Any one or more genomic mutations that result in a stop codon being introduced into the MMA-CoA mutase gene may be used. For example, in some embodiments, the one or more genomic mutations that result in a knock out of the endogenous MMA-CoA mutase gene comprise or consist of G311A and G312A substitutions relative to SEQ ID NO: 3, resulting in the stop codon TAA at positions 310-312 of SEQ ID NO: 3. In some embodiments, the one or more genomic mutations that result in a knock out of the endogenous MMA-CoA mutase gene comprise or consist of a G312A substitution relative to SEQ ID NO: 3, resulting in the stop codon TGA at positions 310-312 of SEQ ID NO: 3. In some embodiments, the mouse is homozygous for the knockout of the endogenous MMA-CoA mutase gene. These mice are designated MUT^{KO/KO} mice herein.

In some embodiments, CRISPR/Cas9-mediated homology-directed repair (HDR) is used to introduce the one or more genomic mutations into the endogenous MMA-CoA mutase gene that results in a knockout of an endogenous MMA-CoA mutase gene. In some embodiments, a single guide RNA (sgRNA) encoded by the polynucleotide sequence of SEQ ID NO: 14 is used for the CRISPR/Cas9-mediated HDR.

In some embodiments, the MUT^{KO/KO} mice, the MUT^{M698K/KO} mice, or the MUT^{M698K/M698K} mice further comprises one or more genomic mutations in an endogenous BCAT2 gene that results in a knockout of the endogenous BCAT2 gene. In some embodiments, the mouse is homozygous for the knockout of the endogenous BCAT2 gene (BCAT2^{KO/KO})

In some embodiments, the mouse comprising the M698K substitution in MMA-CoA mutase and/or knockout of the MMA-CoA mutase gene has increased plasma and/or urine levels of a compound selected from the group consisting of 3-OH-isobutyric acid (3-HIB) and methylmalonic acid (MMA) relative to a syngeneic wildtype mouse.

In some embodiments, the homozygous Mut^{M698K/M698K} mice have increased plasma and urine MMA levels relative to heterozygous Mut^{M698K/WT} mice, or relative to Mut^{WT/WT} mice. In some embodiments, the Mut^{M698K/M698K} mice have at least a 2-fold, 3-fold, 4-fold, 5-fold, 10-fold, 20-fold, 50-fold or 100-fold increase in plasma and/or urine MMA levels relative to heterozygous Mut^{M698K/WT} mice, or relative to Mut^{WT/WT} mice. In some embodiments, the homozygous Mut^{M698K/M698K} mice have at least a 10-fold increase in plasma MMA levels relative to heterozygous Mut^{M698K/WT} mice. In some embodiments, the homozygous Mut^{M698K/M698K} mice have at least a 100-fold increase in plasma MMA levels relative to heterozygous Mut^{M698K/WT} mice. In some embodiments, the homozygous Mut^{M698K/M698K} mice have at least a 10-fold increase in urine MMA levels relative to heterozygous Mut^{M698K/WT} mice. In some embodiments, the homozygous Mut^{M698K/M698K} mice have at least a 50-fold increase in urine MMA levels relative to heterozygous Mut^{M698K/WT} mice.

In some embodiments, heterozygous Mut^{M69K/KO} mice have at least a 2-fold, 3-fold, 4-fold, 5-fold, 10-fold, or 20-fold increase in plasma and/or urine MMA levels relative to homozygous Mut^{M698K/M698K} mice. In some embodiments, heterozygous Mut^{M698K/KO} mice have at least a 2-fold increase in plasma MMA levels relative to homozygous Mut^{M698K/M698K} mice. In some embodiments, heterozygous Mut^{M698K/KO} mice have at least a 10-fold increase in urine MMA levels relative to homozygous Mut^{M698K/M698K} mice.

### PCCA A134T/A134T and A134T/KO

Propionyl-CoA carboxylase (PCC) is a ubiquitously expressed, heteropolymeric mitochondrial enzyme involved primarily in the catabolism of branched-chain amino acids and fatty acids, which harbor odd-numbered chain lengths. PCC contains two subunits, an α subunit (PCCA) and a β subunit (PCCB).

In certain aspects, the disclosure relates to a mouse (Mus musculus) comprising one or more genomic mutations in at least one endogenous propionyl-Coenzyme A carboxylase (PCCA) gene that results in an A134T substitution relative to the amino acid sequence of SEQ ID NO: 6 in a PCCA protein encoded by the gene. In some embodiments, the mouse is an animal model for propionic academia. The A134T substitution may be introduced into PCCA by changing the guanine (G) at position 439 of the nucleic acid sequence of SEQ ID NO: 5 to an adenine (A). This substitution changes the alanine (A) codon GCT at positions 439 to 441 of SEQ ID NO: 5 to the threonine (T) codon ACT. Accordingly, in some embodiments, the one or more genomic mutations comprise or consist of a G439A substitution relative to the nucleic acid sequence of SEQ ID NO: 5. Any substitution in the PCCA gene that results in the A134T substitution in the PCCA protein may be used. For example, in some embodiments, the G439A substitution may be combined with a T441C, T441A or T441G substitution relative to the nucleic acid sequence of SEQ ID NO: 5 to produce the A134T substitution in the PCCA protein.

In some embodiments, the mouse is homozygous for the one or more genomic mutations in the endogenous PCCA gene that results in the A134T substitution. These mice are designated as PCCA^{A134T/A134T} mice herein. In some embodiments, the mouse is heterozygous for the one or more genomic mutations in the endogenous PCCA gene that results in the A134T substitution. These mice are designated as PCCA^{A134T/WT} mice herein.

In some embodiments, the mouse comprises the one or more genomic mutations that results in the A134T substitution at a first locus of an endogenous PCCA gene, and further comprises one or more genomic mutations that result in a knockout of an endogenous PCCA gene at a second locus of an endogenous PCCA gene. In some embodiments, the one or more genomic mutations that result in a knock out of the endogenous PCCA gene are in exon 5 of the endogenous PCCA gene. Exon 5 is nucleotides 328-441 of SEQ ID NO: 5. In some embodiments, the one or more genomic mutations that result in a knock out of the endogenous PCCA gene introduce a premature stop codon into the endogenous PCCA mutase gene. In some embodiments, the one or more genomic mutations that result in a knock out of the endogenous PCCA gene comprise the addition of one or more nucleotides to exon 5 of the endogenous PCCA gene. For example, the knockout in exon 5 may be introduced by adding an additional adenine (A) after the adenine (A) at position 379 of SEQ ID NO: 5. This addition creates a premature stop codon at positions 381-383 of SEQ ID NO: 5. Any mutation that results in a premature stop codon in exon 5 may be used. For example, in some embodiments, addition of a single nucleotide upstream of position 381 of SEQ ID NO: 5 may be used to create the premature stop codon in exon 5. In some embodiments, the one or more genomic mutations that result in a knock out of the endogenous PCCA gene comprise the addition of a nucleotide at position 379 of SEQ ID NO: 5. In some embodiments, the nucleotide is adenine.

In some embodiments, the mouse containing an A134T substitution in PCCA is generated by CRISPR/Cas9-mediated homology-directed repair (HDR) that introduces the one or more genomic mutations into the endogenous PCCA gene that results in an A134T substitution. In some embodiments, a single guide RNA (sgRNA) encoded by the polynucleotide sequence of SEQ ID NO: 31 is used for the CRISPR/Cas9-mediated HDR. In some embodiments, the CRISPR/Cas9-mediated HDR utilizes a donor DNA having the polynucleotide sequence of SEQ ID NO: 32. In some embodiments the mouse containing an A134T substitution in PCCA is generated by CRISPR/Cas9-mediated homology-directed repair (HDR) that introduces the one or more genomic mutations into the endogenous PCCA gene that results in a knockout of an endogenous PCCA gene. In some embodiments, a single guide RNA (sgRNA) encoded by the polynucleotide sequence of SEQ ID NO: 26 is used for the CRISPR/Cas9-mediated HDR.

In some embodiments, the mouse having an A134T substitution in PCCA and/or a knockout of an endogenous PCCA gene has increased plasma propionyl carnitine (PPC) levels relative to a mouse that does not comprise the A134T substitution and/or a knockout in an endogenous PCCA gene. For example, in some embodiments, PCCA^{134T/A134T} mice have increased PPC levels relative to wildtype mice. In some embodiments, the mouse having an A134T substitution in PCCA and/or a knockout of an endogenous PCCA gene has at least a 2-fold, 3-fold, 4-fold, 5-fold, 10-fold or 20-fold increase in plasma PPC levels relative to a wildtype mouse. For example, in some embodiments, homozygous PCCA^{A134T/A134T} mice have at least a 5-fold increase in plasma PPC levels relative to a wildtype mouse.

In some embodiments, the mice described herein are male. In some embodiments, the mice described herein are female. In some embodiments, the mice described herein are C₅₇BL/6 mice.

### Synthesis of Compounds of the Disclosure

Compounds of the disclosure can be prepared by methods described in the General Schemes, Intermediate Schemes, procedures, and Examples set forth within, and by related methods known in the art.

### General Scheme 1A: Preparation of Compounds of Formula I-A and Formula II-A

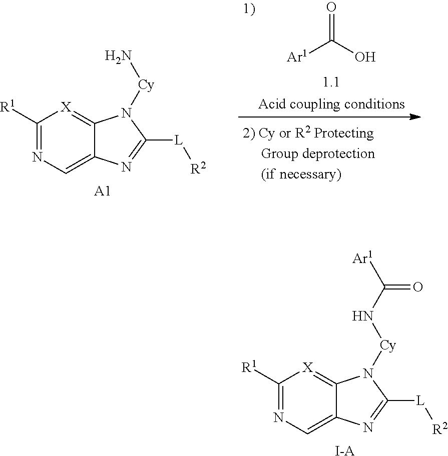

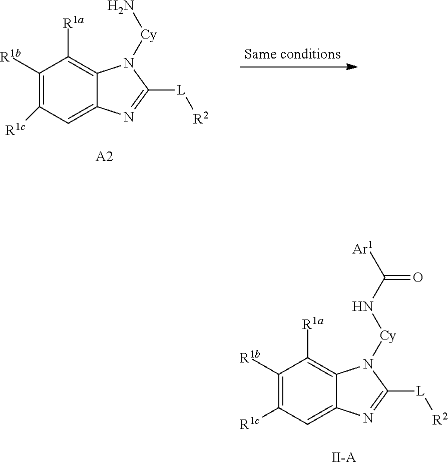

Compounds of formulas I-A and II-A were obtained through General Scheme 1A. Starting with an intermediate of formula A1 or A2, Ar¹ groups were then installed by reacting an Ar¹ carboxylic acid of formula 1.1 or a basic salt (i.e., Li, K, or Na) thereof with the amine of formula A1 or A2 using acid coupling conditions known in the art, such as using HATU or EDCI and HOBt along with a base, such as DIPEA (Hunig's base) or TEA, to afford compounds of Formula I-A or II-A. If the compounds still contain any protecting groups on the Cy or R² groups, they may be removed under acidic conditions such as hydrochloric acid to afford compounds of Formula I-A or II-A.

### General Scheme 1B: Preparation of Formulas I-B and II-B

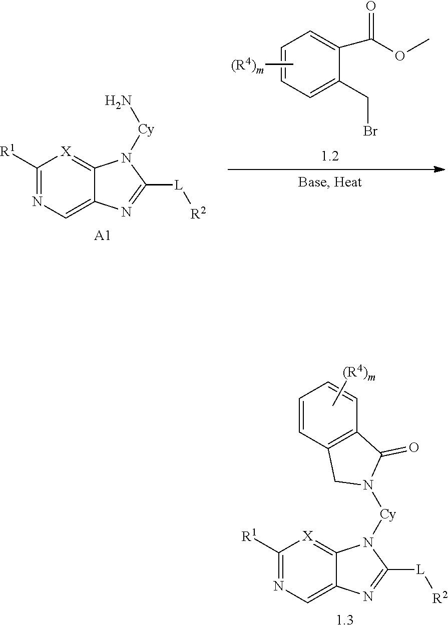

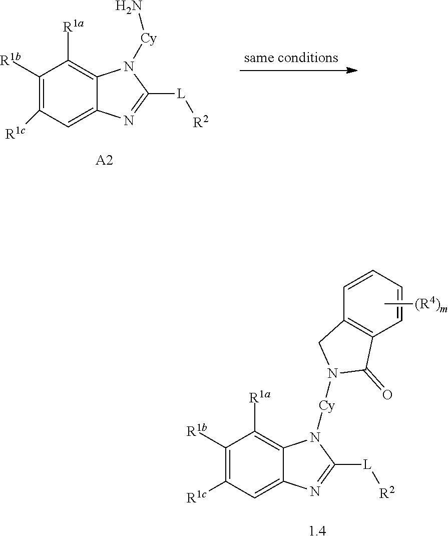

Compounds of formulas I-B and II-B wherein R^{3a} and R^{3b} are each hydrogen were obtained through General Scheme 1B. Starting with an intermediate of formula A1 or A2, the amine in a compound of formula A1 or A2 was condensed with a methyl 2-(bromomethyl)benzoate of formula 1.2 in the presence of a base, such as TEA, and heat, followed by cyclization to afford compounds of formulas 1.3 and 1.4.

### General Scheme 1C: Preparation of Formulas I-C and II-C

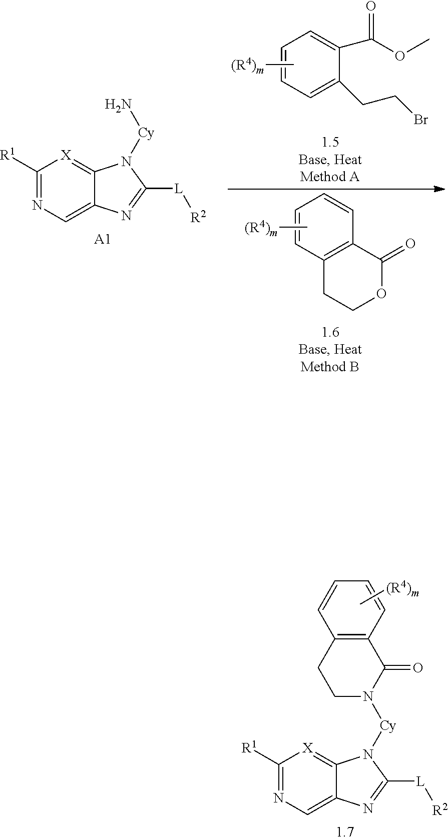

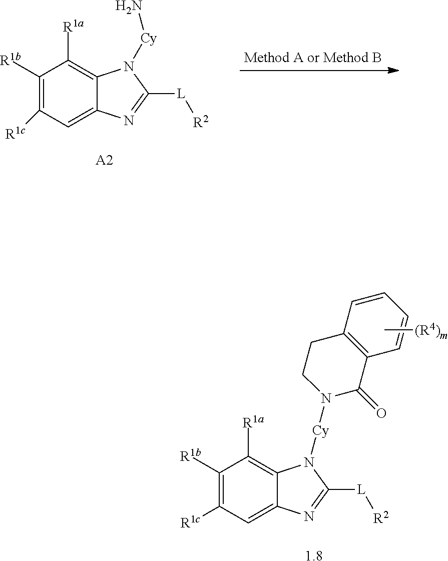

Compounds of formulas I-C and II-C wherein R^{3a} and R^{3b} are each hydrogen were obtained through General Scheme 1C. Starting with an intermediate of formula A1 or A2, the amine in a compound of formula A1 or A2 was condensed with a methyl 2-(2-bromoethyl)benzoate of formula 1.5 (Method A) or an isochroman-1-one of formula 1.6 (Method B) in the presence of a base, such as TEA, and heat, followed by cyclization to afford compounds of formulas 1.7 and 1.8.

Alternatively, in method B, the isochroman-1-one of formula 1.6 may be activated first by reacting it with thionyl chloride and heat to afford a 2-(2-chloroethyl)benzoyl chloride of formula 1.9,

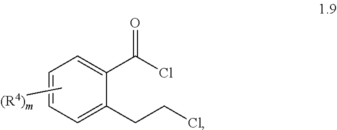

which is then condensed with the amine of formula A1 or A2 in the presence of a base and heat.

### General Scheme 1D: Preparation of Compounds of Formulas I-D and II-D

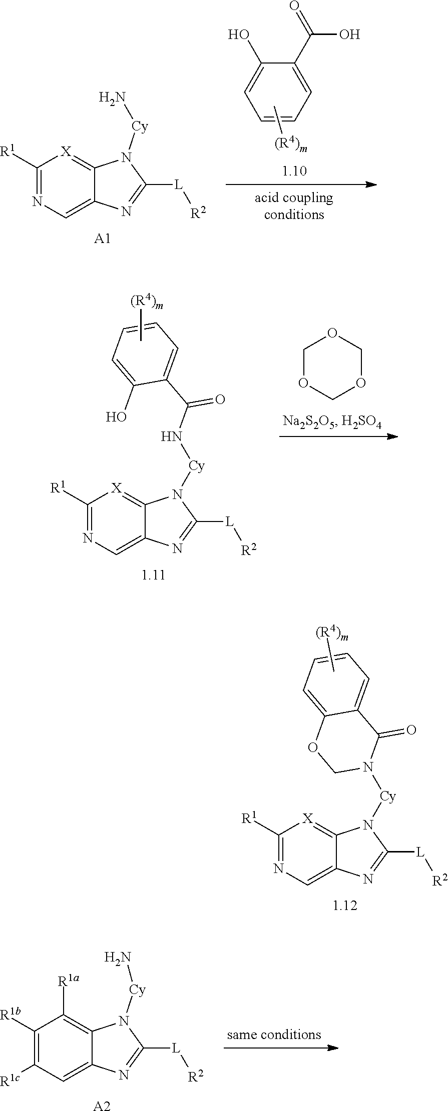

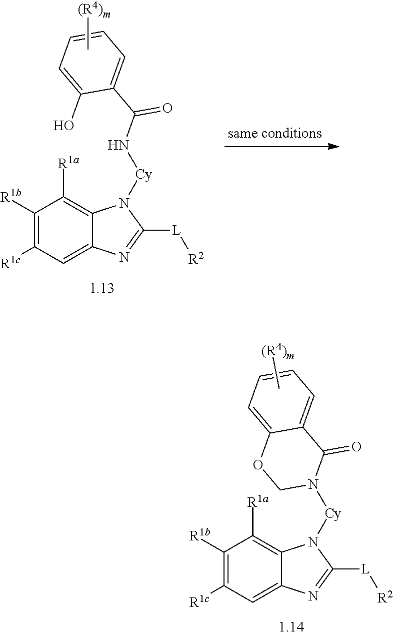

Compounds of formulas I-D and II-D wherein Y is O were obtained through General Scheme 1D. Starting with an intermediate of formula A1 or A2, the amine in a compound of formulas A1 or A2 was reacted with a 2-hydroxybenzoic acid of formula 1.10 using acid coupling conditions known in the art, such as using HATU or EDCI and HOBt along with a base, such as DIPEA (Hunig's base) or TEA, to afford compounds of formulas 1.11 and 1.13. A compound of formula 1.11 or 1.13 was treated with 1,3,5-trioxlane, sodium metabisulfite, and sulfuric acid to afford compounds of formulas 1.12 and 1.14.

### General Scheme 1E: Couplings to Afford Compounds of Formulas I-D and I-E and Formulas II-D and II-E

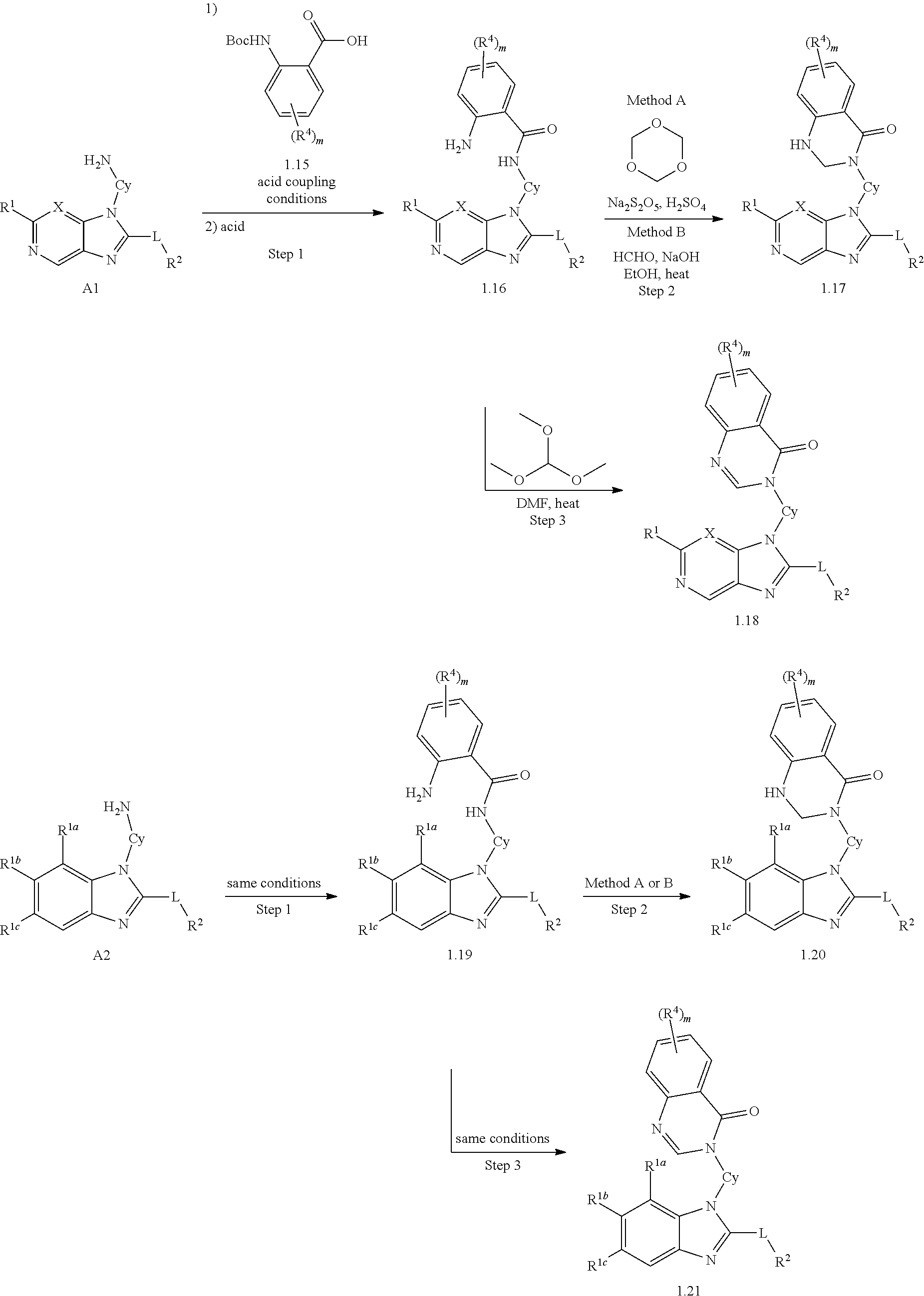

Compounds of formulas I-D and I-E and formulas II-D and II-E, wherein Y is N were obtained through General Scheme 1E. In Step 1, starting with an intermediate of formula A1 or A2, the amine in a compound of formulas A1 or A2 was reacted with a Boc-protected 2-(amino)benzoic acid of formula 1.15 using acid coupling conditions known in the art, such as using HATU or EDCI and HOBt along with a base, such as DIPEA (Hunig's base) or TEA, followed by removal of the boc protecting group under acidic conditions to afford compounds of formulas 1.16 and 1.19. In Step 2, a compound of formula 1.16 or 1.19 is then cyclized using the reagents in Methods A or B to afford compounds of formulas 1.17 and 1.20. In Method A, a compound of formula 1.16 or 1.19 is reacted with 1,3,5-trioxlane, sodium metabisulfite, and sulfuric acid. In Method B, a compound of formula 1.16 or 1.19 is reacted with formaldehyde and a base such as sodium hydroxide in an alcoholic solvent with heat. In Step 3, a compound of formula 1.16 or 1.19 is reacted with trimethoxymethane to afford a compound of formula 1.18 and 1.21.

### General Scheme 1F: Couplings to Afford Compounds of Formulas I-F and II-F

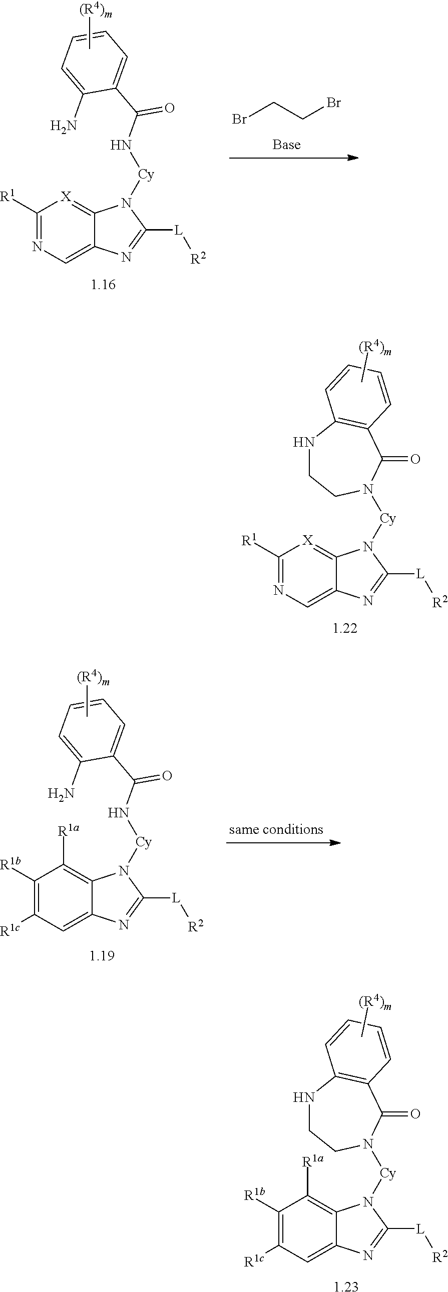

Compounds of formula I-F and formula II-F, wherein Y is N were obtained through General Scheme 1F. Starting with a compound of formula 1.16 or formula 1.19 (prepared in accordance with General Scheme 1E), a compound of formula 1.16 or 1.19 is reacted with 1,3-dibromopropane and a base such as potassium carbonate to afford compounds of formulas 1.22 and 1.23.

### General Scheme 1G: Preparation of Formulas I-B and II-B

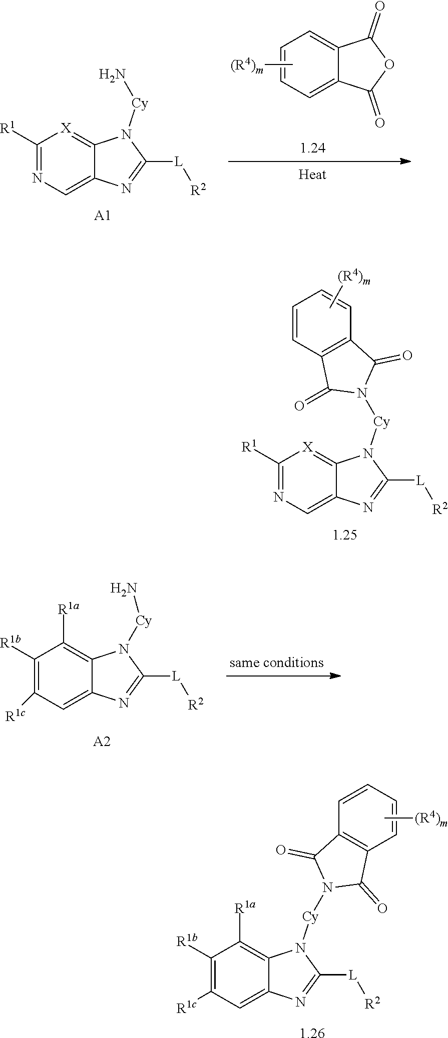

Compounds of formulas I-B and II-B wherein R^{3a} is oxo (═O) and R^{3b} is hydrogen were obtained through General Scheme 1G. Starting with an intermediate of formula A1 or A2, the amine in a compound of formula A1 or A2 was condensed with an isobenzofuran-1,3-dione of formula 1.24 with heat to afford compounds of formulas 1.25 and 1.26.

### General Scheme 1H: Preparation of Formulas I-C and II-C

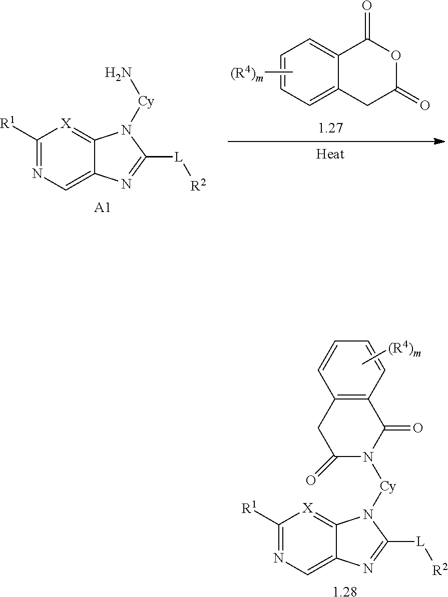

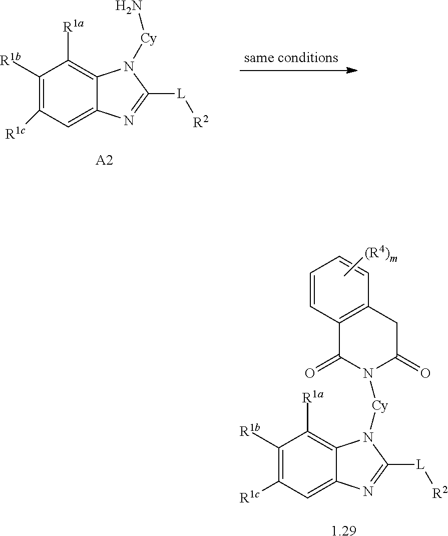

Compounds of formulas I-C and II-C wherein R^{3a} is oxo (═O) and R^{3b} is hydrogen may be obtained through General Scheme 1H. Starting with an intermediate of formula A1 or A2, the amine in a compound of formula A1 or A2 is condensed with a 3,4-dihydro-1H-2-benzopyran-1,3-dione of formula 1.27 with heat to afford compounds of formulas 1.28 and 1.29.

### General Scheme 2A: Preparation of Intermediates of Formula A1

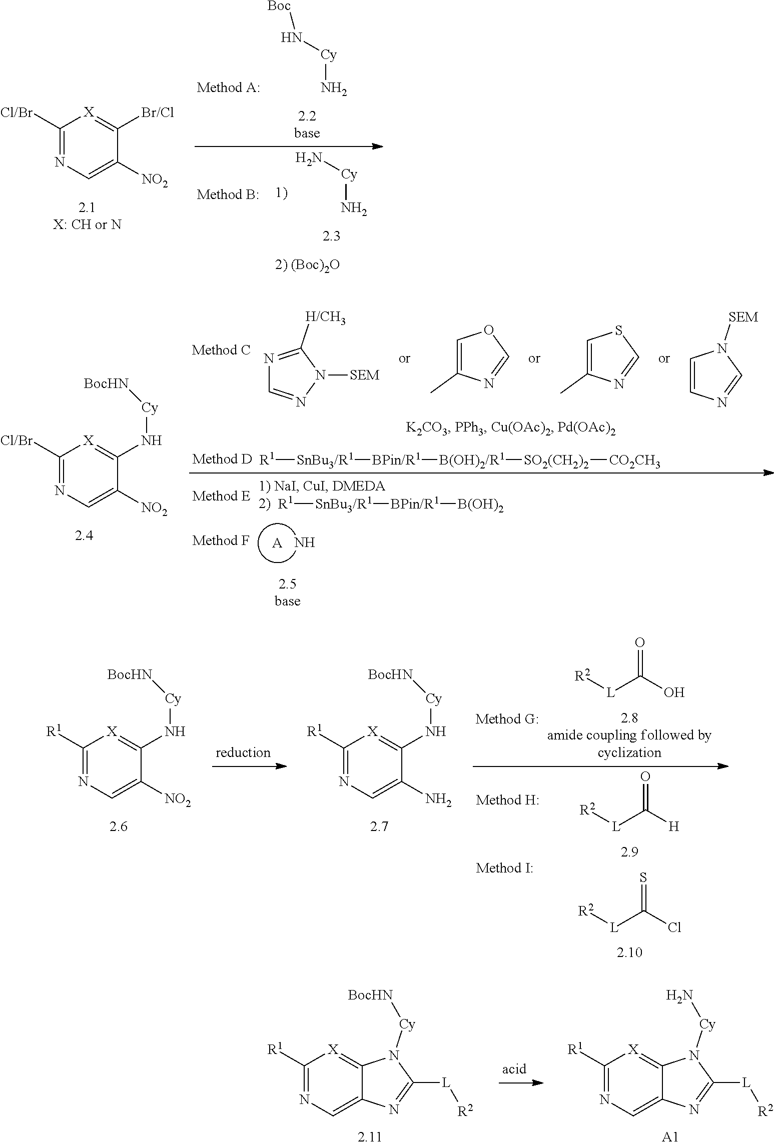

Intermediates of formula A1 were obtained through General Scheme 2A. Beginning with the 2,4-di-halo-5-nitropyridine of formula 2.1, a nucleophilic aromatic substitution reaction conducted under basic conditions was used to couple a N-Boc-1,3-Cy-diamine compound of formula 2.2 (Method A) to afford compounds of formula 2.4. Alternatively, reagent 2.1 underwent selective nucleophilic aromatic substitution by 1,3-Cy-diamine of formula 2.3 (Method B), and the intermediate amine group was protected with a Boc-protecting group (or other suitable nitrogen protecting group) to afford compounds of formula 2.4. The chloride/bromide moiety of a compound of formula 2.4 can be used as a functional handle to install various R¹ aryls, R¹ heteroaryls, and R¹ heterocycloalkyls. Under Method C, R¹ heteroaryls such as unsubstituted and substituted 1,2,4-triazoles, thiazoles, oxazoles, and imidazoles can be installed via a C—H functionalization reaction. Under Method D, the chloride/bromide moiety of a compound of formula 2.4 was converted to a R¹ heteroaryl or aryl using a Stille coupling (R¹—SnBu₃), Suzuki coupling (R¹—BPin or R¹—B(OH)₂), or a cross-coupling with a substituted sulfone (R¹—SO₂(CH₂)₂—CO₂CH₃) to afford compounds of formula 2.6. In some cases, the chloride/bromide moiety needed to be converted to the more reactive iodide moiety using the reaction conditions in Method E prior to the Stille or Suzuki coupling. Under Method F, a heteroaryl or heterocycloalkyl containing at least one nitrogen (formula 2.5) was installed by displacing the bromide or chloride in a compound of formula 2.4 with the NH of the heteroaryl or heterocycloalkyl reagent of formula 2.5 in the presence of a suitable base such as cesium carbonate and/or a metal catalyst such as CuI to form compounds of formula 2.6. The nitro group in a compound of formula 2.6 was reduced with a reducing agent, such as iron in the presence of ammonium chloride or Pd/C, to afford the pyridine-diamine compound of formula 2.7. Subsequently, various L and R² groups were installed by coupling the pyridine-diamine compound of formula 2.7 with an acid of formula 2.8 to form an amide intermediate (Method G), which was then cyclized under either basic or acidic conditions to form compounds of formula 2.11. Alternatively, the pyridine-diamine compound of formula 2.7 was condensed with an aldehyde of formula 2.9 (Method H), followed by cyclization and in situ oxidation to afford compounds of formula 2.11. Alternatively, as shown in Method I, the pyridine-diamine compound of formula 2.7 was condensed with a carbothioyl chloride to form an amide intermediate, which was then cyclized under basic or acidic conditions to form a compound of formula 2.11. The Boc protecting group of a compound of formula 2.11 was then removed under acidic conditions such as TFA or HCl to form intermediates of formula A1. Intermediates of formula A1 may then be converted to compounds of Formula I as set forth in General Schemes 1A through 1H.

### General Scheme 2B: Preparation of Intermediates of Formula A1

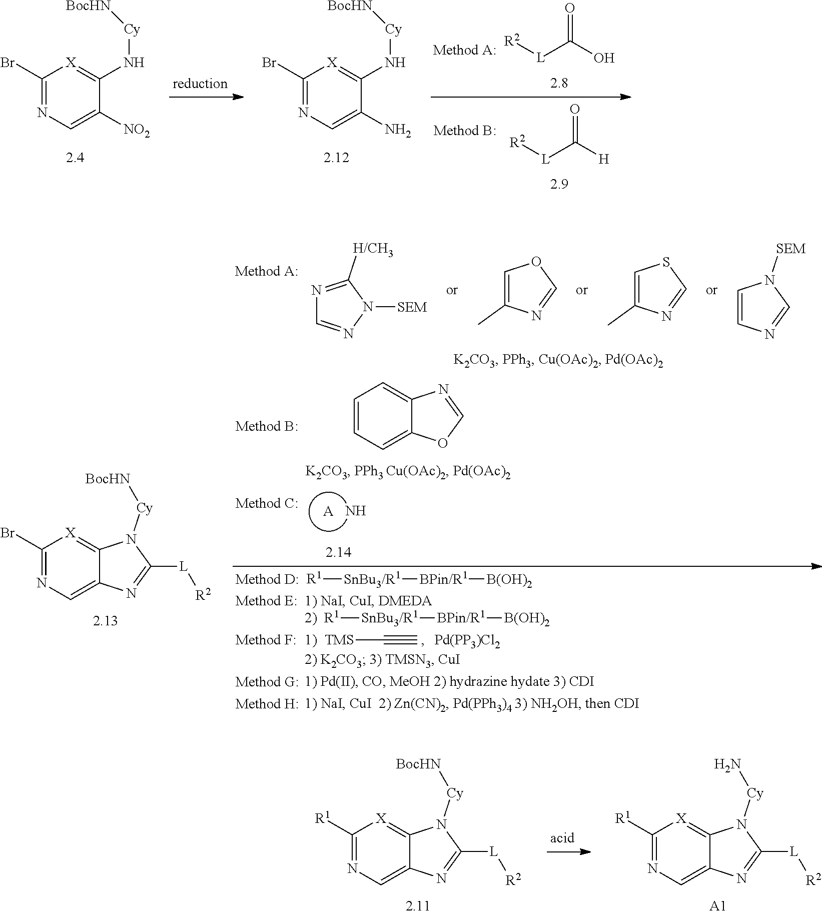

Intermediates of formula A1 were also obtained through General Scheme 2B. The nitro group of intermediate of formula 2.4 (prepared via General Scheme 2A) was reduced with a reducing agent such as iron in the presence of ammonium chloride to afford 2-bromopyridine-diamine compounds of formula 2.12. Subsequently, various L and R² groups were installed by coupling the pyridine-diamine compound of formula 2.12 with an acid of formula 2.8 to form the amide intermediate (Method A), which was subsequently cyclized under either basic or acidic conditions to form compounds of formula 2.13. Alternatively, the pyridine-diamine compound of formula 2.12 was condensed with an aldehyde of formula 2.9 (Method B), followed by cyclization and in situ oxidation to form compounds of formula 2.13. R¹ groups were installed by reacting the bromide in compounds of formula 2.13 with the reagents specified in Methods A, B, C, D, E, F, G, and H to afford compounds of formula 2.11. Under Methods A and B, R¹ heteroaryls such as 1,2,4-triazole, 4-methyloxazole, 4-methylthiazole, imidazole, and benzoxazole were installed via a C—H functionalization reaction of the heteroaryl. Under Method C, the bromide of formula 2.13 was displaced by the NH of heterocyclic amines (saturated or aromatic) (reagent of formula 2.14) in the presence of a suitable base such as cesium carbonate and/or metal catalysts such as CuI. Under Method D, the bromide of formula 2.13 was converted to a R¹ heteroaryl or aryl using a Stille coupling (R¹—SnBu₃) or Suzuki coupling (R¹—BPin or R¹—B(OH)₂) to afford compounds of formula 2.11. In some cases, the bromide moiety needed to be converted to the more reactive iodide moiety using the reaction conditions in Method E prior to the Stille or Suzuki coupling. Under Method F, a 1,2,3-triazole was installed by (1) converting the bromide of formula 2.13 to an ethynyltrimethylsilane moiety, (2) removing the trimethylsilane group under basic conditions such as potassium carbonate, and (3) reacting the intermediate with TMSN₃ in the presence of a metal catalyst such as CuI. Under Method G, an 1,3,4-oxadiazol-2-one was installed by converting the bromide moiety to a methyl ester, followed by reacting the methyl ester with hydrazine hydrate and cyclizing with CDI. Under Method H, an 1,2,4-oxadiazol-one was installed by converting the bromide moiety of formula 2.13 to an iodide moiety, which was then subsequently converted to a cyano moiety via reaction with Zn(CN)₂ and Pd(PPh₃)₄. The cyano moiety was then reacted hydroxylamine and cyclized with CDI. The Boc protecting group in compounds of formula 2.11 was removed under acidic conditions such as TFA or HCl to afford intermediates of formula A1. Intermediates of formula A1 may then be converted to compounds of Formula I as set forth in General Schemes 1A through 1H.

### General Scheme 2C: Preparation of Intermediates of Formula A1

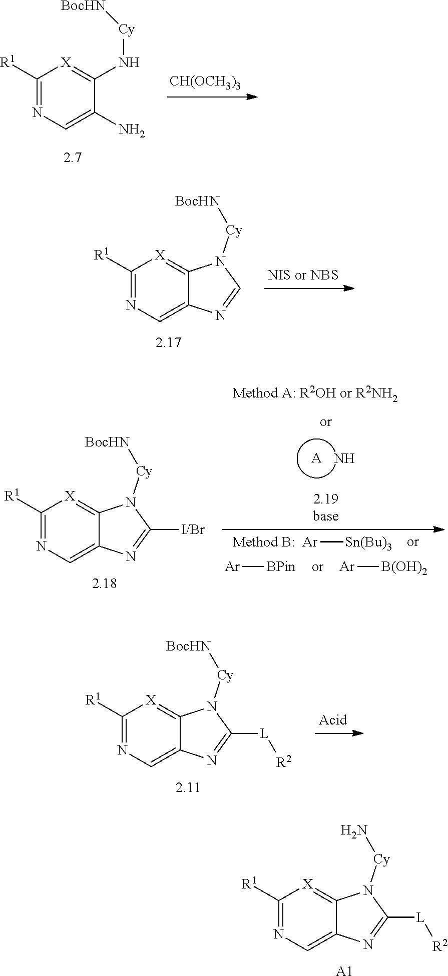

Intermediates of formula A1 were obtained through General Scheme 2C. The pyridine-diamine compound of formula 2.7 (made in accordance with General Scheme 2A) was reacted with trimethyl orthoformate to form the imidazole of azabenzimidazole compound of formula 2.17. An iodide or bromide was installed at the 2-position of the azabenzimidazole compound of formula 2.17 using a bromination or iodination reagent to afford compounds of formula 2.18. Subsequently, various L and R² groups were installed by displacing the halide in compounds of formula 2.18 with an R²-alcohol or R²-primary amine or heterocyclic amine of formula 2.19 (Method A), wherein the heterocyclic ring can be saturated or aromatic, to afford compounds of formula 2.11. Alternatively, using Method B, the halide in compound of formula 2.18 was converted to an aryl or R¹ heteroaryl group using a Stille coupling (R¹—SnBu₃) or a Suzuki coupling (R¹—BPin or R¹—B(OH)₂) to afford compounds of formula 2.11. The amino Boc protecting group in compounds of formula 2.11 was removed under acidic conditions to afford intermediates of formula A1. Intermediates of formula A1 may then be converted to compounds of Formula I as set forth in General Schemes 1A through 1H.

### General Scheme 2D: Preparation of Intermediates of Formula 2.11

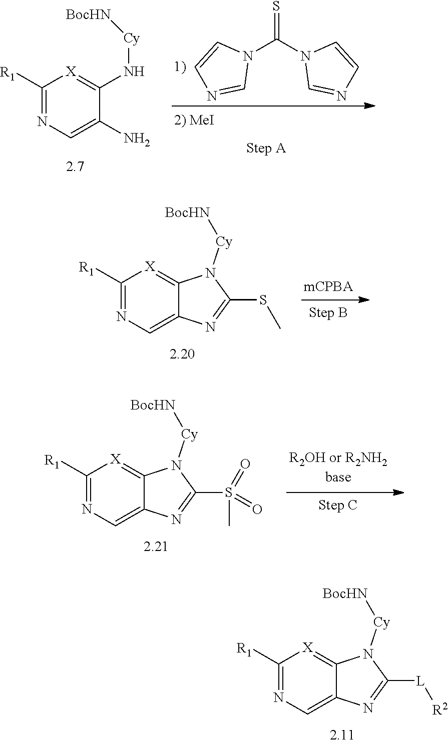

Compounds of formula 2.11 wherein L is —O— or —NH— may also be obtained through General Scheme 2D. In step A, the pyridine-diamine compound of formula 2.7 (made in accordance with General Scheme 2A) was first reacted with thiocarbonyliimidazole, and then methyl iodide to afford compounds of formula 2.20. In step B, the 2-methylthio moiety of formula 2.20 was oxidized with mCPBA to afford compounds of formula 2.21. In step C, the methylsulfonyl moiety of formula 2.21 was then displaced by an R²-alcohol or R²-primary amine (wherein R² is a heteroaryl or heterocycloalkyl) in the presence of a base such as cesium carbonate to afford compounds of formula 2.11. Compounds of formula 2.11 may then be converted to compounds of Formula I wherein L is —O— or —NH— as set forth in General Schemes 1A through 1H.

Alternatively, Steps A, B, and C of General Scheme 2D can be carried out starting from compounds of formula 2.12

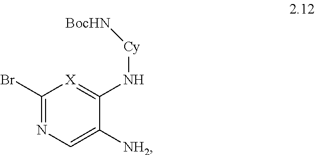

prepared in accordance with General Scheme 2B, and then the R¹ group is installed using Methods A, B, C, D, E, F, G, and H from General Scheme 2B, before the additional steps of General Scheme 2C are carried out.

### General Scheme 3: Preparation of Intermediates of Formula 3.4

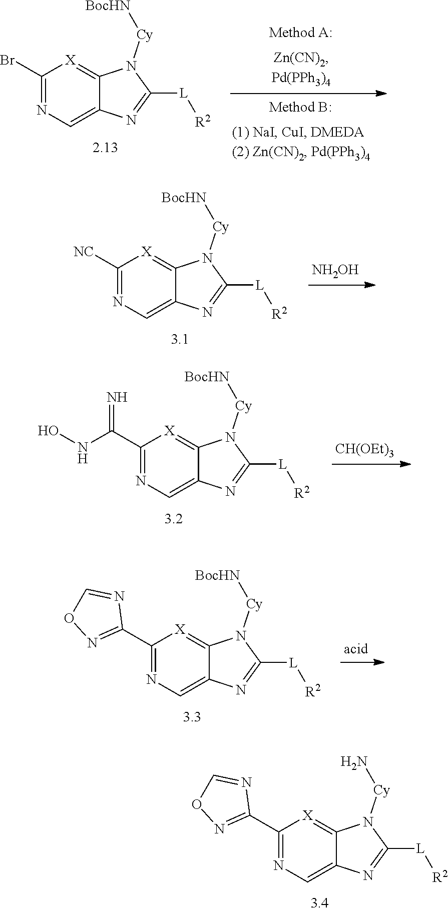

Intermediates of formula A1 wherein R¹ is a 1,2,4-oxadiazole may be obtained through General Scheme 3. The bromide in the 2-bromopyridine-diamine compounds of formula 2.13 (prepared via General Scheme 2B) can be converted to a cyano group either directly (Method A) or by first converting the bromide to an iodide, followed by cyanation (Method B) to afford compounds of formula 3.1. The cyano moiety of a compound of formula 3.1 can be converted to a 1,2,4-oxadiazole using hydroxylamine hydrochloride followed by triethyl orthoformate to afford compounds of formula 3.3. The Boc protecting group of compounds of formula 3.3 can be removed under acidic conditions such as TFA or HCl to afford compounds of formula 3.4. Compounds of formula 3.4 may then be converted to compounds of Formula I as set forth in General Schemes 1A through 1H.

### General Scheme 4: Preparation of Intermediate of Formula 4.2

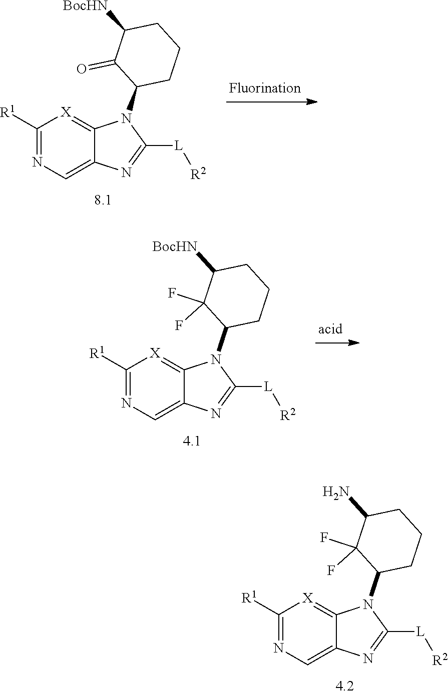

Compounds of formula 4.2 can be obtained through General Scheme 4 (relative stereochemistry is indicated in General Scheme 4). A compound of formula 8.1 (see General Scheme 8) is fluorinated with a suitable fluorination agent to afford compounds of formula 4.1. The Boc protecting group of a compound of formula 4.1 is then removed under acidic conditions such as TFA or HCl to form a compound of formula 4.2. Compounds of formula 4.2 may then be converted to compounds of Formula I as set forth in General Schemes 1A through 1H.

### General Scheme 5: Preparation of Compounds of Formula I-A

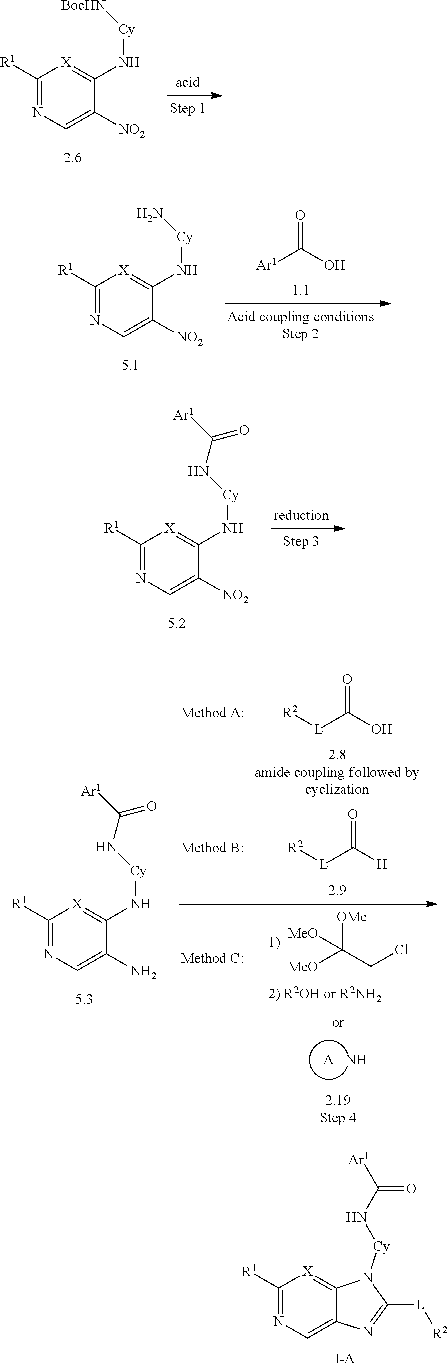

Compounds of Formula I-A may also be obtained through General Scheme 5. In step 1, the Boc protecting group of a compound of formula 2.6 (prepared according to General Scheme 2A) is removed under acidic conditions such as TFA or HCl to generate compounds of formula 5.1. In step 2, Ar¹ groups are then installed by reacting an Ar¹ carboxylic acid of formula 1.1 or a basic salt (i.e., Li, K, or Na) thereof with the amine of formula 5.1 using acid coupling conditions known in the art, such as using HATU or EDCI and HOBt along with a base, such as DIPEA (Hunig's base) or TEA to afford compounds of formula 5.2. In step 3, the nitro group in compounds of formula 5.2 is reduced with a reducing reagent such as iron in the presence of ammonium chloride or Pd/C to generate the pyridine-diamine compound of formula 5.3. In step 4, various L and R² groups are installed by coupling the pyridine-diamine compound of formula 5.3 with an acid of formula 2.8 to form an amide intermediate (Method A), which is subsequently cyclized under either basic or acidic conditions to form compounds of Formula I-A. Alternatively, the pyridine-diamine compound of formula 5.3 is condensed with an aldehyde of formula 2.9 (Method B), followed by cyclization and in situ oxidation to afford compounds of Formula I-A. Alternatively, the pyridine-diamine of formula 5.3 could be condensed with 2-chloro-1,1,1-trimethoxyethane to afford the 2-chloromethyl imidazole intermediate (Method C), followed by displacement of the chloride with an R²-alcohol or R²-primary amine or heterocyclic amine of formula 2.19 wherein the heterocyclic ring is either saturated or aromatic, to afford compounds of Formula I-A.

### General Scheme 6: Preparation of Compounds of Formula I-A

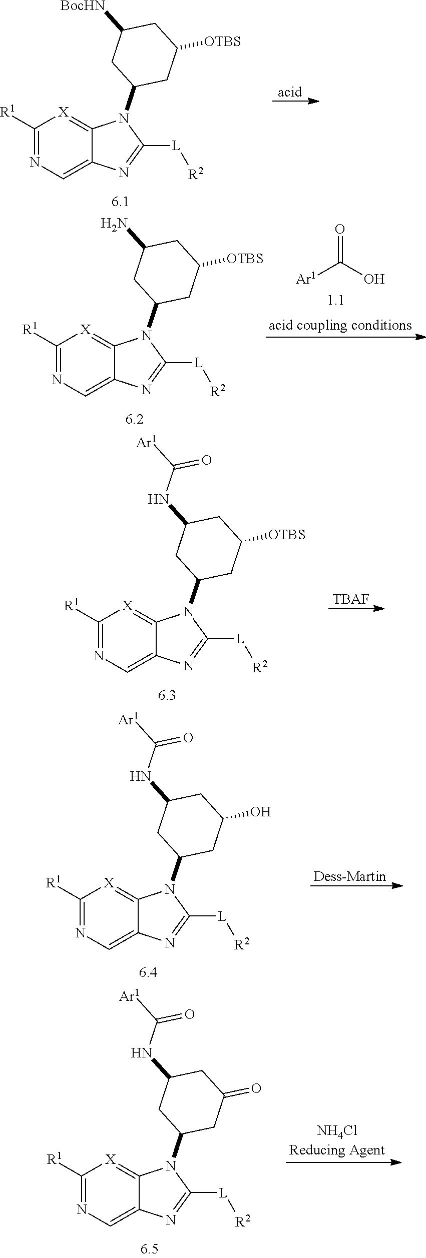

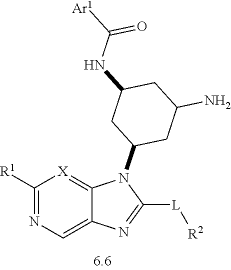

Compounds of formula 6.6 were obtained through General Scheme 6 (relative stereochemistry is indicated in General Scheme 6). Beginning with a compound of formula 6.1, the Boc protecting group was removed under acidic conditions such as TFA to form a compound of formula 6.2. Ar¹ groups were then installed by reacting an Ar¹ carboxylic acid of formula 1.1 or a basic salt (i.e., Li, K, or Na) thereof with the amine of formula 6.2 using acid coupling conditions known in the art, such as using HATU or EDCI and HOBt along with a base, such as DIPEA (Hunig's base), pyridine, or TEA to afford compounds of formula 6.3. The TBS protecting group of a compound of formula 6.3 was removed with TBAF to afford compounds of formula 6.4. The hydroxyl moiety of a compound of formula 6.4 was oxidized to a ketone moiety via reaction with the Dess-Martin reagent or other suitable oxidation agent to afford a compound of formula 6.5. A compound of formula 6.5 then underwent a reductive animation reaction with ammonium chloride and a reducing agent such as NaBH₃CN to afford compounds of formula 6.6.

Compounds of formula 6.2 may also be coupled with the methyl 2-(bromomethyl)benzoate of formula 1.2 (Scheme 1B), the methyl 2-(2-bromoethyl)benzoate of formula 1.5 (Scheme 1C), the isochroman-1-one of formula 1.6 (Scheme 1C), the 2-hydroxybenzoic acid of formula 1.10 (Scheme 1D), the reagents in Schemes 1E and 1F, the isobenzofuran-1,3-dione of formula 1.24 (Scheme 1G), the 3,4-dihydro-1H-2-benzopyran-1,3-dione of formula 1.27 (Scheme 1H) under the conditions described in Schemes 1B to 1H to afford compounds of formulas 1.3, 1.7, 1.12, 1.17, 1.18, 1.22, 1.25, and 1.28.

### General Scheme 7: Preparation of Compounds of Formula I

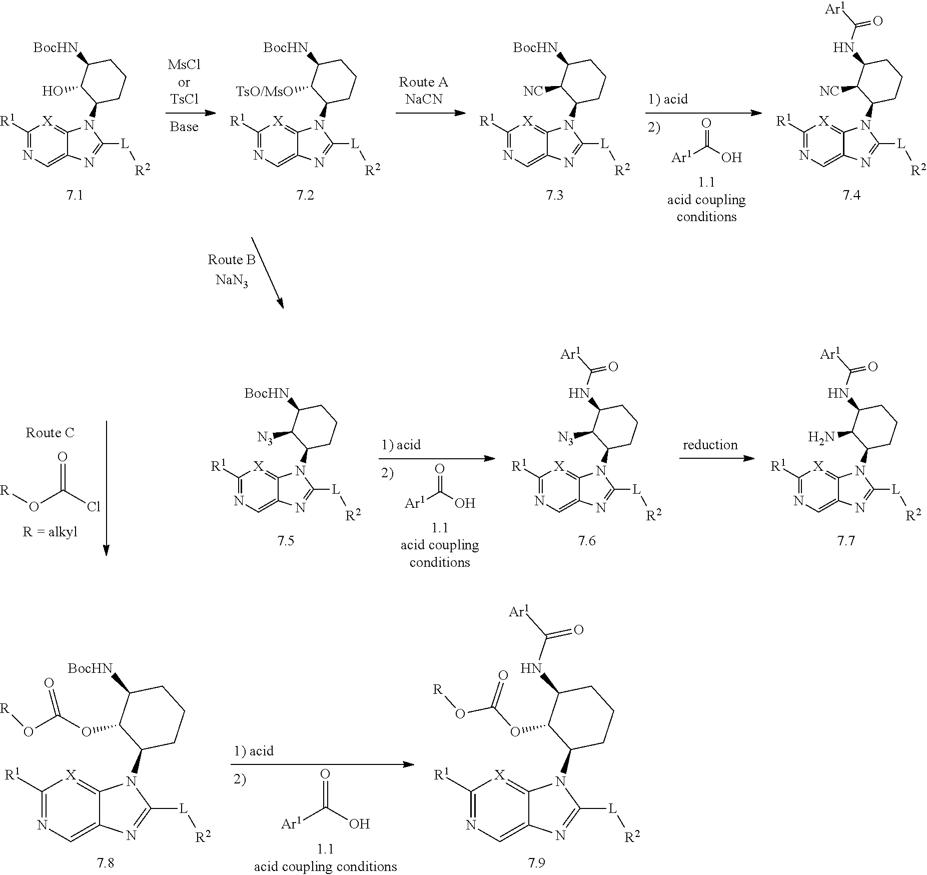

Compounds of formula 7.4, 7.7, and 7.9 were obtained through General Scheme 7. Under Routes A and B, the hydroxyl moiety of a compound of formula 7.1 was converted to a tosylate or mesylate moiety by reacting a compound of formula 7.1 with mesyl chloride or tosyl chloride in the presence of base such as TEA or NaH, to afford compounds of formula 7.2. Under Route A, a compound of formula 7.2 is reacted with sodium cyanide in a SN₂ reaction to afford a compound of formula 7.3. Under Route B, a compound of formula 7.2 is reacted with sodium azide in a SN₂ reaction to afford a compound of formula 7.5. Under Route C, a compound of formula 7.1 is reacted with an alkyl carbonochloride to afford a compound of formula 7.8. The Boc protecting groups in compounds of formulas 7.3, 7.5, and 7.8 were removed under acidic conditions, and then Ar¹ groups were then installed by reacting an Ar¹ carboxylic acid of formula 1.1 or a basic salt (i.e., Li, K, or Na) thereof with the unprotected amine using acid coupling conditions known in the art, such as using HATU or EDCI and HOBt along with a base, such as DIPEA (Hunig's base) or TEA to afford compounds of formula 7.4, 7.6, and 7.9. The azido moiety in compounds of formula 7.6 can be reduced to an amino moiety under reduction conditions such as triphenylphosphine in THF/water or Pd/C in the presence of H₂ gas to afford compounds of formula 7.7.

Alternatively, once the Boc protecting groups in compounds of formulas 7.3, 7.5, and 7.8 are removed under acidic conditions, the resulting unprotected amine may be coupled with the methyl 2-(bromomethyl)benzoate of formula 1.2 (Scheme 1B), the methyl 2-(2-bromoethyl)benzoate of formula 1.5 (Scheme 1C), the isochroman-1-one of formula 1.6 (Scheme 1C), the 2-hydroxybenzoic acid of formula 1.10 (Scheme 1D), the reagents in Schemes 1E and 1F, the isobenzofuran-1,3-dione of formula 1.24 (Scheme 1G), the 3,4-dihydro-1H-2-benzopyran-1,3-dione of formula 1.27 (Scheme 1H) under the conditions described in Schemes 1B to 1H to afford compounds of formulas 1.3, 1.7, 1.12, 1.17, 1.18, 1.22, 1.25, and 1.28.

### General Scheme 8: Preparation of Compounds of Formula I-A

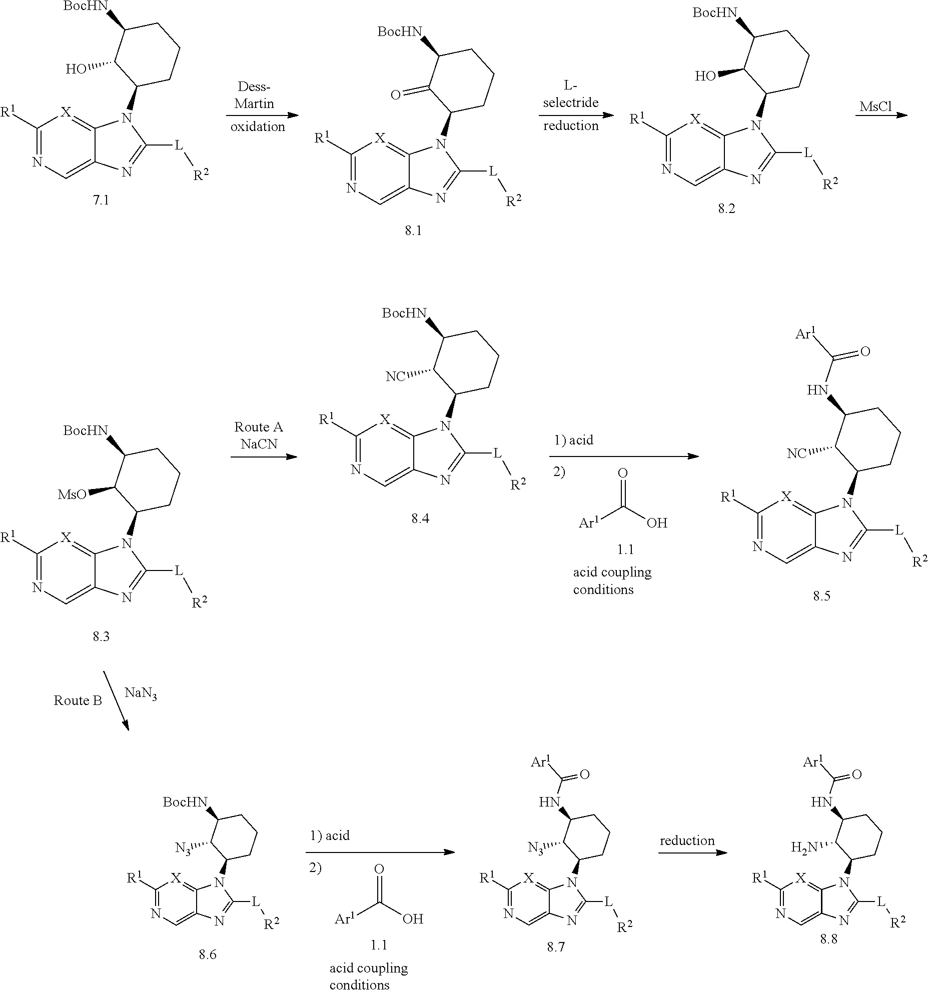

Compounds of formula 8.5, 8.7, and 8.8 were obtained through General Scheme 8. The stereochemistry of the hydroxyl moiety of a compound of formula 7.1 was inverted by first using a Dess-Martin oxidization reaction to afford a compound of formula 8.1, followed by a reduction reaction using L-selectride to afford compounds of formula 8.2. The hydroxyl moiety of compounds of formula 8.2 was converted to a mesylate moiety by reacting a compound of formula 8.2 with mesyl chloride in the presence of base to afford compounds of formula 8.3. Under Route A, a compound of formula 8.3 is reacted with sodium cyanide in a SN₂ reaction to afford a compound of formula 8.4. Under Route B, a compound of formula 8.3 is reacted with sodium azide in a SN₂ reaction to afford a compound of formula 8.6. The Boc protecting groups in compounds of formulas 8.4 and 8.6 were removed under acidic conditions, and then Ar¹ groups were then installed by reacting an Ar¹ carboxylic acid of formula 1.1 or a basic salt (i.e., Li, K, or Na) thereof with the unprotected amine using acid coupling conditions known in the art, such as using HATU or EDCI and HOBt along with a base, such as DIPEA (Hunig's base) or TEA to afford compounds of formula 8.5 and 8.7. The azido moiety in compounds of formula 8.7 can be reduced to an amino moiety under reduction conditions such as triphenylphosphine in THF/water or Pd/C in the presence of H₂ gas to afford compounds of formula 8.8.

Alternatively, once the Boc protecting groups in compounds of formulas 8.4 and 8.6 are removed under acidic conditions, the resulting unprotected amine may be coupled with the methyl 2-(bromomethyl)benzoate of formula 1.2 (Scheme 1B), the methyl 2-(2-bromoethyl)benzoate of formula 1.5 (Scheme 1C), the isochroman-1-one of formula 1.6 (Scheme 1C), the 2-hydroxybenzoic acid of formula 1.10 (Scheme 1D), the reagents in Schemes 1E and 1F, the isobenzofuran-1,3-dione of formula 1.24 (Scheme 1G), the 3,4-dihydro-1H-2-benzopyran-1,3-dione of formula 1.27 (Scheme 1H) under the conditions described in Schemes 1B to 1H to afford compounds of formulas 1.3, 1.7, 1.12, 1.17, 1.18, 1.22, 1.25, and 1.28.

### General Scheme 9: Preparation of Intermediates of Formula A2

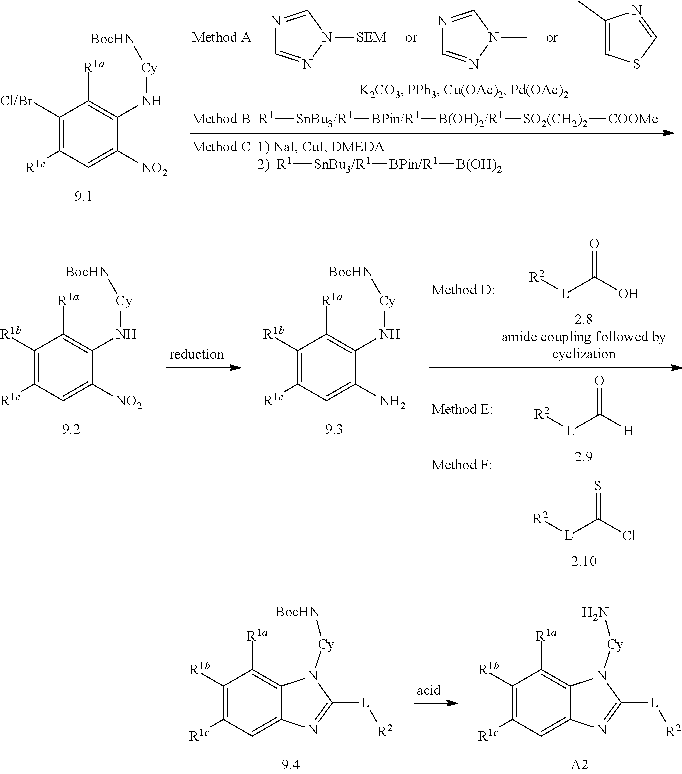

Intermediates of formula A2 were obtained through General Scheme 9. The chloride/bromide moiety of a compound of formula 9.1 can be used as a functional handle to install various R¹ aryls, R¹ heteroaryls, and R¹ heterocycloalkyls. Under Method A, unsubstituted and substituted 1,2,4-triazoles, thiazoles, and oxazoles can be installed via a C—H functionalization reaction of the heteroaryl. Under Method B, the chloride/bromide moiety of a compound of formula 9.1 was converted to a R¹ heteroaryl or aryl using a Stille coupling (R¹—SnBu₃), Suzuki coupling (R¹-BPin or R¹—B(OH)₂), or a cross-coupling with a substituted sulfone (R¹—SO₂(CH₂)₂—CO₂CH₃) to afford compounds of formula 9.2. In some cases, the chloride/bromide moiety needed to be converted to the more reactive iodide moiety using the reaction conditions in Method C prior to the Stille or Suzuki coupling. The nitro group in a compound of formula 9.2 was reduced using a reducing reagent such as iron in the presence of ammonium chloride or Pd/C to afford the pyridine-diamine compounds of formula 9.3. Subsequently, various L and R² groups were installed by coupling the pyridine-diamine compound of formula 9.3 with a L-R² carboxylic acid of formula 2.8 to form an amide intermediate (Method D), which was then cyclized under either basic or acidic conditions to form a compound of formula 9.4. Alternatively, the pyridine-diamine compound of formula 9.3 was condensed with an aldehyde of formula 2.9 (Method E), followed by cyclization and in situ oxidation to afford a compound of formula 9.4. Alternatively, as shown in Method F, the pyridine-diamine compound of formula 9.3 was condensed with a carbothioyl chloride of formula 2.10 to form an amide intermediate, which was then cyclized under basic or acidic conditions to form a compound of formula 9.4. The Boc protecting group of a compound of formula 9.4 was then removed under acidic conditions such as TFA or HCl to form intermediates of formula A2. Intermediates of formula A2 may then be converted to compounds of Formula II as set forth in General Schemes 1A through 1H.

The present disclosure will be more fully understood by reference to the Examples described herein. The examples should not, however, be construed as limiting the scope of the present disclosure.

### Units and Terms List

| | |
|---|---|
| anhy. | anhydrous |
| aq. | aqueous |
| atm | atmosphere |
| min | minute(s) |
| hrs | hours |
| mL | milliliter |
| μL | microliter |
| mmol | millimole(s) |
| μmol | micromole(s) |
| mol | mole(s) |
| MS | mass spectrometry |
| LC-MS | Liquid chromatography-mass spectrometry |
| NMR | nuclear magnetic resonance |
| TLC | thin layer chromatography |
| HPLC | high-performance liquid chromatography |
| sat. | saturated |
| °C | degrees Celsius |
| rt | room temperature |
| wt | weight |
| N₂ | nitrogen gas |
| H₂ | hydrogen gas |

### NMR Spectra

| | |
|---|---|
| Hz | hertz |
| δ | chemical shift |
| s | singlet |
| d | doublet |
| t | triplet |
| q | quartet |
| m | multiplet |
| br | broad |
| dd | doublet of doublets |
| CDCl₃ | chloroform-d |
| CD₃OD | methanol-d₄ |
| DMSO-d₆ | dimethylsulfoxide-d₆ |
| CD₃CN | acetonitrile-d₃ |

### Solvents and Reagents:

| | |
|---|---|
| CHCl₃ | chloroform |
| DCM | dichloromethane |
| DCE | 1,2-dichloroethane |
| DMF | dimethylformamide |
| EtOH | ethyl alcohol |
| EtOAc | ethyl acetate |
| MeOH | methyl alcohol |
| MeCN or ACN | acetonitrile |
| PE | petroleum ether |
| THF | tetrahydrofuran |
| DMSO | dimethyl sulfoxide |
| t-BuOH | tert-butanol |
| AcOH | acetic acid |
| HCl | hydrochloric acid |
| H₂SO₄ | sulfuric acid |
| FA | formic acid |
| NH₄C1 | ammonium chloride |
| NaOH | sodium hydroxide |
| LiOH | lithium hydroxide |
| K₂CO₃ | potassium carbonate |
| Na₂CO₃ | sodium carbonate |
| Cs₂CO₃ | cesium carbonate |
| TFA | trifluoroacetic acid |
| Na₂SO₄ | sodium sulfate |
| Na₂CO₃ | sodium carbonate |
| NaHCO₃ | sodium bicarbonate |
| K₃PO₄ | potassium phosphate |
| NaH | sodium hydride |
| MeONa | sodium methoxide |
| MeMgBr | methylmagnesium bromide |
| CH₃I or MeI | methyl iodide |
| NH₄OH | ammonium hydroxide |
| Et₃N or TEA | triethylamine |
| DIEA | N,N-diisopropylethylamine |
| DIPA | diisopropylamine |
| DMEDA | N,N′-Dimethylethylenediamine |
| Py | pyridine |
| EDCI | 1-ethyl-3-(3-dimethylaminopropyl)carbodiimide |
| HOBt | 1-hydroxybenzotriazole |
| HATU | 1-[Bis(dimethylamino)methylene]-1H-1,2,3- |
| | triazolo[4,5-b]pyridinium 3-oxid hexafluorophosphate |
| CDI | carbonyldiimidazole |
| DBU | 1,8-diazabicyclo[5.4.0]undec-7-ene |
| Boc₂O | di-tert-butyl dicarbonate |
| H₂O | water |
| K₂OsO₄•2H₂O | potassium osmate(VI) dihydrate |
| Fe | iron |
| PPh₃ | triphenyl phosphine |
| Pd/C | palladium on carbon |
| Pd(PPh₃)₄ | tetrakis(triphenylphosphine)palladium(0) |
| Pd(PPh₃)₂Cl₂ | bis(triphenylphosphine)palladium(II) dichloride |
| Pd(OAc)₂ | palladium (II) acetate |
| Pd(dppf)Cl₂ | (1,1′-Bis(diphenylphosphino)ferrocene)palladium(II) |
| | dichloride |
| t-BuXphos-Pd-G3 | [(2-Di-tert-butylphosphino-2′,4′,6′-triisopropyl-1,1′- |
| | biphenyl)-2-(2′-amino-1,1′-biphenyl)] palladium(II) |
| | methanesulfonate |
| Cu(OAc)₂ | copper (II) acetate |
| CuI | copper iodine |
| CuCl | copper chloride |
| NaN₃ | sodium azide |
| Na₂O₅S₂ | sodium metabisulfite |
| SOCl₂ | thionyl chloride |
| NaI | sodium iodide |
| Zn(CN)₂ | zinc cyanide |
| TBDPSCl | tert-butyldiphenylsilyl chloride |
| TsCl | p-toluenesulfonyl chloride |
| DEAD | diethyl azodicarboxylate |
| TBAF | tetra-n-butylammonium fluoride |
| TMSN₃ | trimethylsilyl azide |

### General Experimental

In the following examples, the reagents and solvents were purchased from commercial sources (such as Alfa, Acros, AstaTech, CombiBlocks, Enamine, Sigma Aldrich, TCI, PharmaBock, Bide Pharmatech Ltd., Accela ChemBio, Aladdin, Shanghai Haohong Pharmaceutical Co., Ltd, Amkchem, Beijing Ouhe Technology Co., Ltd, Haoyuan Chemexpress Co., Ltd, Hualun, Coolpharm, Scochem, Titan and WuXi LabNetwork), and used without further purification unless otherwise specified. Flash chromatography was performed on a CombiFlashRf 150 (ISCO), CombiFlash R^{f}+, or CombiFlash R^{f}+Lumen via columns with silica gel particles of 200-300 mesh. HPLC was performed on an Agilent 1100 Liquid Chromatography (Agilent, USA), a Shimadzu L C 20/20A (Shimadzu, Japan), and a Waters Acquity UPLC (Waters, USA). Supercritical fluid chromatography was performed on a Waters Prep SFC 150 AP/80Q/200/350 system (Waters, USA) or a SHIMADZU Prep-UC (Shimadzu, Japan). Analytical and preparative thin layer chromatography plates (TLC) were HSGF 254 (0.15-0.2 mm thickness, Shanghai Anbang Company, China). Nuclear magnetic resonance (NMR) spectra were obtained on a Brucker AV-400 NMR (Bruker, Switzerland). Chemical shifts were reported in parts per million (ppm, δ) downfield from tetramethylsilane. Mass spectra were given with electrospray ionization (ESI) from a Waters LCT TOF Mass Spectrometer (Waters, USA). LC-MS was performed on an Agilent Prime-6125B/Agilent LC1260-MS6150/Agilent LC1260-MS6125B/Agilent LC1200-MS6110 (Agilent, USA), a Shimadzu LC20-MS2020, and a Waters Acquity UPLC-H Class. Microwave reactions were run on an Initiator 2.5 Microwave Synthesizer (Biotage, Sweden). Lyophilizations were done on EYELA Freeze Dryer FDU-2100 (TOKYO RIKAKIKAI, Japan) or FreeZone 6 Liter Freeze Dryer (LABCONCO, USA).

### Preparation of intermediates of formula 2.2 and formula 2.3

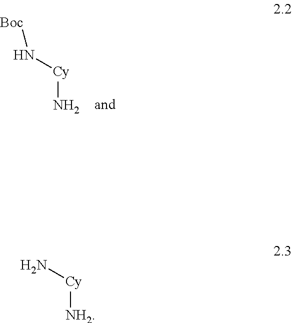

The following Intermediate Schemes 1 to 16 describe the syntheses of intermediates of formula 2.2 or formula 2.3. Additional intermediates of formulas 2.2 and 2.3 are commercially available such as, for example, tert-butyl ((1S,3R)-3-aminocyclohexyl)carbamate, tert-butyl (3-aminocyclohexyl)carbamate, and tert-butyl (5-aminotetrahydro-2H-pyran-3-yl)carbamate.

### Intermediate Scheme 1. Preparation of intermediate tert-butyl (3-amino-2-hydroxycyclohexyl)carbamate (relative stereochemistry is indicated in Intermediate Scheme 1)

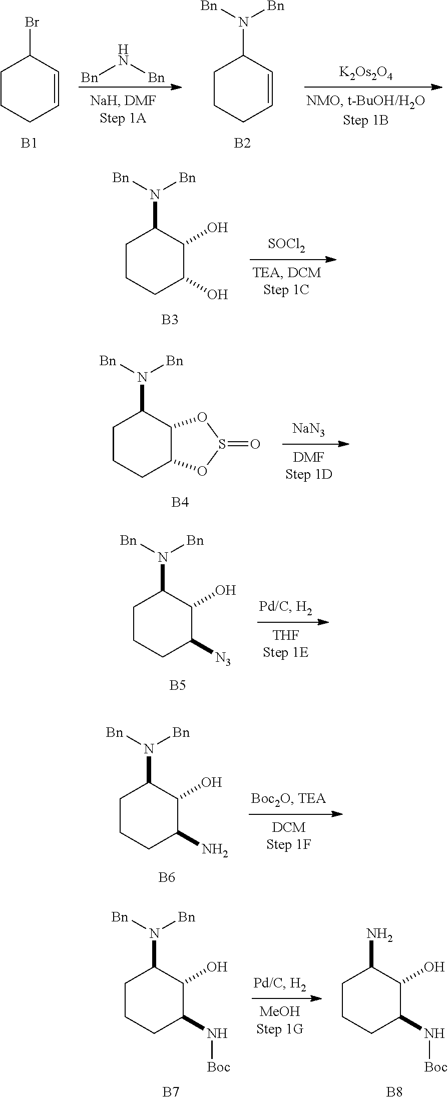

### Step 1A. Synthesis of N,N-dibenzylcyclohex-2-en-1-amine (B2)

A mixture of N-benzyl-1-phenyl-methanamine (3.0 g, 15.2 mmol, 2.91 mL) and 3-bromocyclohexene (B1) (2.45 g, 15.2 mmol, 1.75 mL) in DMF (12 mL) was degassed and purged with N₂ gas 3 times, and then NaH (639 mg, 16.0 mmol, 60% purity) was added to the reaction mixture at 0° C. The reaction mixture was stirred at rt for 12 hrs under N₂ atmosphere, then quenched by the addition of H₂O (15 mL) at 0° C., followed by the addition of additional H₂O (200 mL), and the aqueous portion extracted with EtOAc (200 mL×3). The combined organic layers were washed with brine (300 mL×3), dried over Na₂SO₄, filtered and concentrated to dryness. The residue was purified by silica gel column chromatography to give B2 (5.3 g, 62.8% yield). LC-MS: m/z 278.2 [M+H]⁺.

### Step 1B. Synthesis of 3-(dibenzylamino)cyclohexane-1,2-diol (B3)

To a mixture of N,N-dibenzylcyclohex-2-en-1-amine (B2) (1.93 g, 6.96 mmol) in t-BuOH (40 mL) and H₂O (8 mL) were added K₂OsO₄.2H₂O (28.2 mg, 76.5 μmol) and 4-methyl-4-oxido-morpholin-4-ium (897 mg, 7.65 mmol). The reaction mixture was stirred at rt for 14 hrs. The reaction mixture was treated with sat. aq. sodium hydrosulfite solution (5 mL) and stirred for 1 hr, and then concentrated under reduced pressure. The aqueous portion was extracted with DCM (100 mL×3). The combined organic layers were concentrated to dryness, and the residue was purified by silica gel column chromatography to give B3 (1.63 g, 75.2% yield). LC-MS: m/z 312.2 [M+H]⁺.

### Step 1C. Synthesis of 4-(dibenzylamino)hexahydrobenzo[d][1,3,2]dioxathiole 2-oxide (B4)

To a solution of 3-(dibenzylamino)cyclohexane-1,2-diol (B3) (1.63 g, 5.23 mmol) in DCM (10 mL) was added Et₃N (4.24 g, 41.9 mmol). SOCl₂ (1.87 g, 15.7 mmol) was then added slowly to the reaction mixture at 0° C. The reaction mixture was stirred at 0° C. to rt for 2 hrs, then poured into H₂O (80 mL) at 5° C. Additional water (20 mL) was added, and the aqueous portion extracted with DCM (80 mL×3). The combined organic layers were washed with brine (80 mL×3), dried over Na₂SO₄, filtered, and concentrated to dryness. The residue was purified by silica gel column chromatography to give B4 (940 mg, 50.2% yield). LC-MS: m/z 358.2 [M+H]⁺.

### Step 1D. Synthesis of 2-azido-6-(dibenzylamino)cyclohexan-1-ol (B5)

To a solution of 4-(dibenzylamino)hexahydrobenzo[d][1,3,2]dioxathiole 2-oxide (B4) (100 mg, 280 μmol) in DMF (2 mL) was added NaN₃ (54.6 mg, 839 μmol) at rt. The reaction mixture was heated at 100° C. for 30 hrs. The reaction mixture was cooled to rt, quenched by the addition of brine (10 mL) at 15° C., then H₂O (10 mL) was added, and the aqueous portion extracted with DCM (20 mL×3). The combined organic layers were washed with brine (20 mL×3), dried over Na₂SO₄, filtered, and concentrated to dryness. The residue was purified by silica gel column chromatography to give B5 (55 mg, 58.4% yield). LC-MS: m/z 337.2 [M+H]⁺.

### Step 1E. Synthesis of 2-amino-6-(dibenzylamino)cyclohexan-1-ol (B6)

To a solution of 2-azido-6-(dibenzylamino)cyclohexan-1-ol (B5) (342 mg, 1.02 mmol) in THF (1 mL) was added Pd/C (50 mg, 1.02 mmol, 10% purity) under N₂ atmosphere. The suspension was degassed and purged with H₂ 3 times. The reaction mixture was stirred under H₂ (15 psi) at rt for 1.2 hrs. The reaction mixture was filtered, and the filter cake washed with THF (8 mL×3). The combined organic layers were concentrated to dryness to give B6 (321 mg), which was used without further purification. LC-MS: m/z 311.2 [M+H]⁺.

### Step 1F. Synthesis of tert-butyl (3-(dibenzylamino)-2-hydroxycyclohexyl)carbamate (B7)

To a solution of 2-amino-6-(dibenzylamino)cyclohexan-1-ol (B6) (321 mg, 1.03 mmol) in DCM (4 mL) were added Et₃N (314 mg, 3.10 mmol) and tert-butoxycarbonyl tert-butyl carbonate (237 mg, 1.09 mmol). The reaction mixture was stirred at rt for 13 hrs. The reaction mixture was quenched by the addition of H₂O (5 mL) at 15° C., diluted with H₂O (15 mL) and the aqueous portion extracted with DCM (20 mL×3). The combined organic layers were washed with brine (20 mL×3), dried over Na₂SO₄, filtered, and concentrated to dryness. The residue was purified by silica gel column chromatography to give B7 (350 mg, 82.5% yield). LC-MS: m/z 411.3 [M+H]⁺.

### Step 1G. Synthesis of tert-butyl (3-amino-2-hydroxycyclohexyl)carbamate (B8)

To a solution of tert-butyl (3-(dibenzylamino)-2-hydroxycyclohexyl)carbamate (B7) (350 mg, 853 μmol) in MeOH (5 mL) was added Pd/C (100 mg, 10% Pd) under N₂ atmosphere. The suspension was degassed and purged with H₂ 3 times. The reaction mixture was stirred under H₂ (15 Psi) at rt for 3 hrs. The reaction mixture was filtered, and the filter cake was washed with MeOH (10 mL×3). The combined organic layers were concentrated to dryness to give B8 (202 mg), which was used without further purification. ¹H NMR (400 MHz, CDCl₃) δ 5.11 (s, 1H), 3.40 (s, 1H), 3.24 (s, 1H), 2.87 (s, 1H), 1.98 (br d, 2H), 1.71 (br dd, 1H), 1.44 (s, 9H), 1.38-1.20 (m, 3H).

### Intermediate Scheme 2: Preparation of Intermediate 5-methoxycyclohexane-1,3-diamine (Relative Stereochemistry is Indicated in Intermediate Scheme 2)

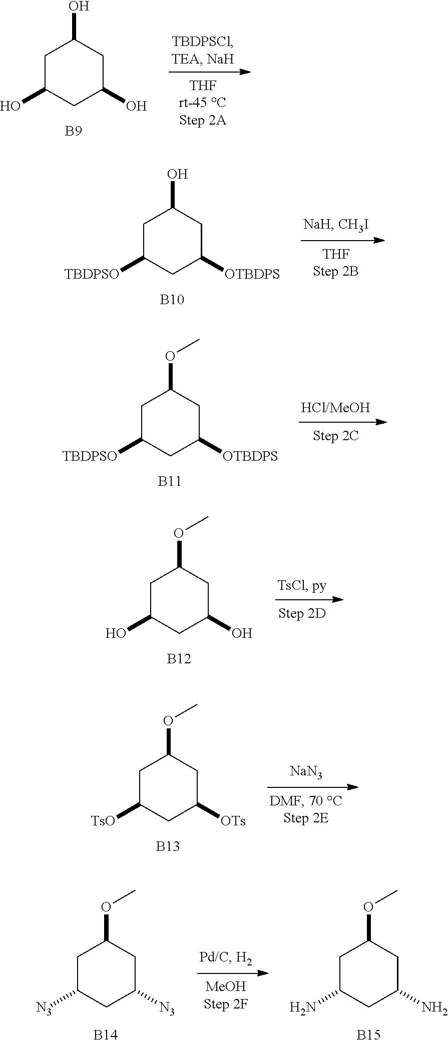

### Step 2A. Synthesis of 3,5-bis((tert-butyldiphenylsilyl)oxy)cyclohexan-1-ol (B10)

To a solution of cis, cis-cyclohexane-1,3,5-triol (B9) (5 g, 37.8 mmol) in THF (100 mL) was added TBDPSCl (21.84 g, 79.5 mmol, 20.4 mL) and TEA (11.49 g, 114 mmol, 15.8 mL) at rt. After addition, the reaction mixture was stirred at rt for 1.5 hrs and treated with NaH (4.54 g, 114 mmol, 60% purity). The reaction mixture was heated to 45° C. for 22.5 hrs under N₂ atmosphere. The reaction mixture was concentrated under reduced pressure. Sat. aq. NH₄Cl solution (50 mL) was added to the reaction mixture, and the aqueous portion was extracted with EtOAc (100 mL×3). The combined organic layers were washed with brine (20 ml), dried over Na₂SO₄, filtered, and concentrated to dryness. The residue was purified by flash silica gel chromatography to give B10 (8.7 g, 35.0% yield). LC-MS: m/z 609.2 (M+H)⁺.

### Step 2B. Synthesis of ((5-methoxycyclohexane-1,3-diyl)bis(oxy))bis(tert-butyldiphenylsilane) (B11)

To a solution of 3,5-bis((tert-butyldiphenylsilyl)oxy)cyclohexan-1-ol (B10) (1.0 g, 1.64 mmol) in THF (12 mL) was added NaH (98.5 mg, 2.46 mmol, 60% purity). The reaction mixture was stirred at rt for 30 min and treated with CH₃I (280 mg, 1.97 mmol, 123 μL). The reaction mixture was stirred at rt for 16 hrs under N₂ atmosphere. The reaction mixture was concentrated under reduced pressure. Sat. aq. NH₄Cl solution (15 mL) was added, and the aqueous portion extracted with EtOAc (30 mL×3). The combined organic layers were washed with brine (20 ml), dried over Na₂SO₄, filtered, and concentrated to dryness. The residue was purified by flash silica gel chromatography to give B11 (1.0 g, 96.8% yield). LC-MS: m/z 623.3 (M+H)⁺.

### Step 2C. Synthesis of 5-methoxycyclohexane-1,3-diol (B12)

To a solution of ((5-methoxycyclohexane-1,3-diyl)bis(oxy))bis(tert-butyldiphenylsilane) (B11) (700 mg, 1.12 mmol) in MeOH (7 mL) was added HCl/MeOH (4 M, 7 mL). The reaction mixture was stirred at rt for 15 hrs and concentrated to dryness to afford B12 (170 mg). LC-MS: m/z 147.1 (M+H)⁺.

### Step 2D. Synthesis of 5-methoxycyclohexane-1,3-diyl bis(4-methylbenzenesulfonate) (B13)

To a solution of 5-methoxycyclohexane-1,3-diol (B12) (170 mg, 1.16 mmol) in pyridine (7 mL) was added TsCl (887 mg, 4.65 mmol). The reaction mixture was stirred at rt for 63 hrs. The reaction mixture was concentrated to dryness. The residue was purified by flash silica gel chromatography to give B13 (440 mg, 67.4% yield). LC-MS: m/z 477.1 (M+Na)⁺.

### Step 2E. Synthesis of 1,3-diazido-5-methoxycyclohexane (B14)

To a solution of 5-methoxycyclohexane-1,3-diyl bis(4-methylbenzenesulfonate) (B13) (440 mg, 968 μmol) in DMF (5 mL) was added NaN₃ (378 mg, 5.81 mmol). The reaction mixture was stirred at 70° C. for 15 hrs. The reaction mixture was cooled to rt, and EtOAc (100 mL) was added. The organic layer was washed with H₂O (40 mL×5), brine (30 mL), dried over Na₂SO₄, filtered, and concentrated to dryness to afford B14 (180 mg). ¹H NMR (400 MHz, CDCl₃) δ 3.75-3.61 (m, 3H), 3.32 (s, 3H), 2.35-2.21 (m, 3H), 1.41-1.30 (m, 3H).

### Step 2F. Synthesis of 5-methoxycyclohexane-1,3-diamine (B15)

To a solution of 1,3-diazido-5-methoxycyclohexane (B14) (180 mg, 917 μmol) in MeOH (2 mL) was added Pd/C (30 mg, 917 μmol, 10% purity) under N₂ atmosphere. The reaction mixture was degassed and purged with H₂ 3 times and stirred under H₂ (15 Psi) at rt for 15 hrs. The reaction mixture was filtered through Celite, and the filtrate concentrated to dryness to afford B15 (90 mg). ¹H NMR (400 MHz, CDCl₃) δ 3.61-3.56 (m, 1H), 3.25 (s, 3H), 2.99-2.93 (m, 2H), 2.02-1.94 (m, 3H), 1.04-0.97 (m, 2H), 0.88-0.79 (m, 1H).

Alternatively, 5-methoxycyclohexane-1,3-diamine (B15) may be prepared in accordance with Intermediate Scheme 2A (relative stereochemistry is indicated in Intermediate Scheme 2A). Steps 2E and 2F were performed following the procedures set forth in the Electronic Supplementary Materials (Scheme S1) for Dalton Trans., 2019, 48, 9576-9580. Steps 2G and 2H were performed following the procedures set forth in New J. Chem., 2005, 29, 1152-1158 (Scheme 2).

### Intermediate Scheme 2A

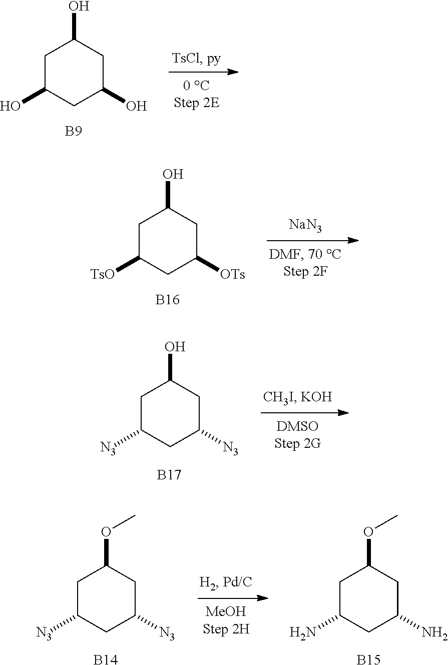

### Intermediate Scheme 3: Preparation of intermediate tert-butyl-3-amino-2-methoxycyclohexylcarbamate (relative stereochemistry is indicated in Intermediate Scheme 3)

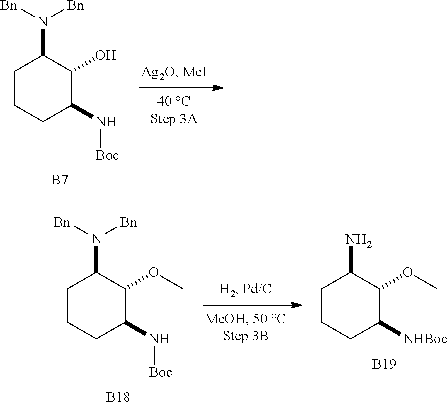

### Step 3A. Synthesis of tert-butyl-(3-(dibenzylamino)-2-methoxycyclohexyl)carbamate (B18)

To a solution of tert-butyl (3-(dibenzylamino)-2-hydroxycyclohexyl)carbamate (B7) (1.70 g, 4.14 mmol) in CH₃I (20 mL) was added silver oxide (2.88 g, 12.4 mmol). The reaction mixture was then degassed under vacuum and purged with N₂ several times, and heated at 40° C. for 48 hrs under N₂ atmosphere in a sealed tube. The reaction mixture was cooled to rt, filtered through a pad of Celite, and the filter cake was washed with EtOAc (20 mL). The filtrate was concentrated to dryness to afford B18 (1.72 g), which was used without further purification. LC-MS: m/z 425 [M+H]⁺.

### Step 3B. Synthesis of tert-butyl-3-amino-2-methoxycyclohexylcarbamate (B19)

To a solution of tert-butyl-(3-(dibenzylamino)-2-methoxycyclohexyl)carbamate (B18) (1.72 g, 4.05 mmol) in MeOH (10 mL) was added Pd/C (431 mg, 10% wt) under N₂ atmosphere. The reaction mixture was degassed and purged with H₂ several times, then heated at 50° C. for 18 hrs under H₂ atmosphere. The reaction mixture was cooled to rt, filtered through a pad of Celite, and the filter cake was washed with MeOH (20 mL). The filtrate was concentrated to dryness to afford B19 (857 mg), which was used without further purification. LC-MS: m/z 245 [M+H]⁺.

### Intermediate Scheme 4: Preparation of intermediates tert-butyl ((1R,3S,5S)-3-((2-bromo-5-nitropyridin-4-yl)amino)-5-methoxycyclohexyl)carbamate and tert-butyl ((1S,3R,5R)-3-((2-bromo-5-nitropyridin-4-yl)amino)-5-methoxycyclohexyl)carbamate (relative stereochemistry is indicated for Steps 4A-4H)

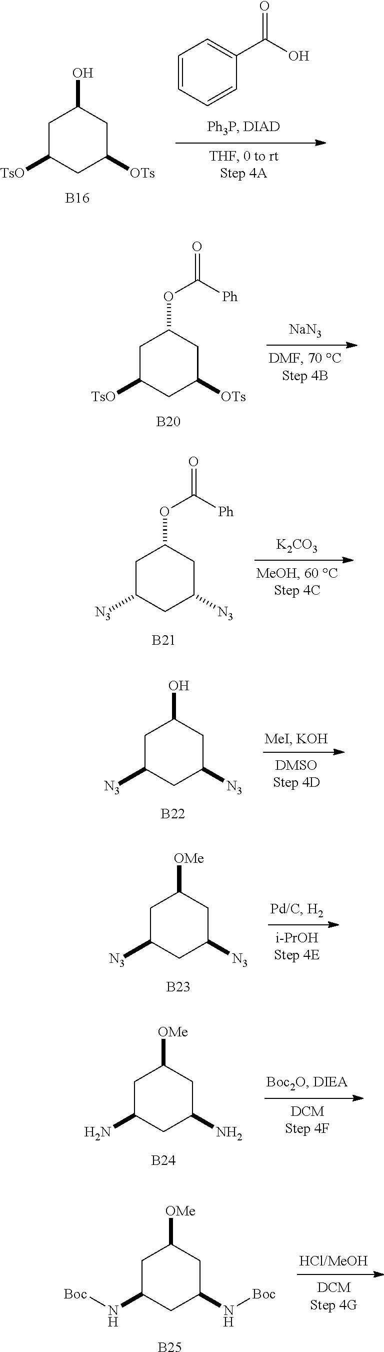

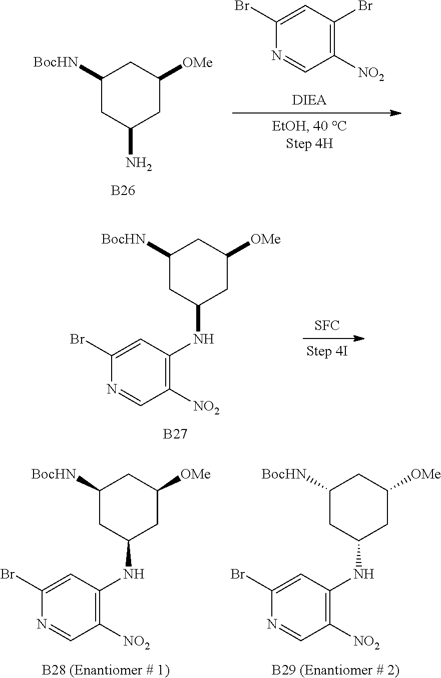

### Step 4A. Synthesis of 3,5-bis(tosyloxy)cyclohexyl benzoate (B20)

To a solution of cis-5-hydroxycyclohexane-1,3-diyl bis(4-methylbenzenesulfonate) (B16) (15 g, 34 mmol) and benzoic acid (5.0 g, 41 mmol) in THF (100 mL) were added Ph₃P (13 g, 51 mmol), and then DIAD (10 g, 51 mmol) at 0° C. The reaction mixture was warmed up to rt and stirred for 16 hrs. Water (200 mL) was added, and the aqueous portion extracted with EtOAc (200 mL×3). The combined organic layers were washed with brine (100 mL), dried over Na₂SO₄, and concentrated to dryness. The residue was purified by flash column chromatography on silica gel to give B20 (10 g, 54% yield). LC-MS: m/z 545 [M+H]⁺.

### Step 4B. Synthesis of 3,5-diazidocyclohexyl benzoate (B21)

To a solution of 3,5-bis(tosyloxy)cyclohexyl benzoate (B20) (10 g, 18 mmol) in DMF (100 mL) was added sodium azide (4.2 g, 64 mmol). The reaction mixture was stirred at 70° C. for 16 hrs and then cooled to rt. Water (300 mL) was added, and the aqueous portion extracted with EtOAc (100 mL×3). The combined organic layers were washed with brine (100 mL), dried over Na₂SO₄, and concentrated to dryness. The residue was purified by flash column chromatography on silica gel to give B21 (4.5 g, 86% yield). LC-MS: m/z 287 [M+H]⁺.

### Step 4C. Synthesis of 3,5-diazidocyclohexan-1-ol (B22)

To a solution of 3,5-diazidocyclohexyl benzoate (B21) (4.5 g, 16 mmol) in MeOH (100 mL) was added K₂CO₃ (6.5 g, 47 mmol). The reaction mixture was stirred at 60° C. for 16 hrs and then cooled to rt. Water (100 mL) was added, and the aqueous portion extracted with EtOAc (100 mL×3). The combined organic layers were washed with brine (100 mL), dried over Na₂SO₄, and concentrated to dryness. The residue was purified by flash column chromatography on silica gel to give B22 (2.5 g, 87% yield). LC-MS: m/z 183 [M+H]⁺.

### Step 4D. Synthesis of 1,3-diazido-5-methoxycyclohexane (B23)

To a solution of 3,5-diazidocyclohexan-1-ol (B22) (2.5 g, 14 mmol) in DMSO (10 mL) were added KOH (2.3 g, 41 mmol) and Mel (5.8 g, 41 mmol). The reaction mixture was stirred at rt for 16 hrs. Water (100 mL) was added, and the aqueous portion extracted with EtOAc (100 mL×3). The combined organic layers were washed with brine (100 mL), dried over Na₂SO₄, and concentrated to dryness. The residue was purified by flash column chromatography on silica gel to give B23 (2.3 g, 85% yield). LC-MS: m/z 197 [M+H]⁺.

### Step 4E. Synthesis of 5-methoxycyclohexane-1,3-diamine (B24)

To a solution of 1,3-diazido-5-methoxycyclohexane (B23) (2.3 g, 12 mmol) in i-PrOH (20 mL) was added Pd/C (230 mg) under nitrogen. The reaction mixture was degassed under vacuum and purged with H₂ several times and then stirred at rt for 16 hrs. The reaction mixture was filtered through a pad of Celite®, and the filter cake was washed with MeOH (20 mL). The combined filtrates were concentrated to dryness to give B24 (1.6 g, 95% yield). LCMS: m/z 145 [M+H]⁺.

### Step 4F. Synthesis of di-tert-butyl-(5-methoxycyclohexane-1,3-diyl)dicarbamate (B25)

To a solution of 5-methoxycyclohexane-1,3-diamine (B24) (2.0 g, 13.9 mmol) and DIEA (5.4 g, 41.7 mmol) in DCM (50 mL) was added Boc₂O (9.09 g, 41.7 mmol). The reaction mixture was stirred at rt for 16 hrs. and then concentrated to dryness. Water (100 mL) was added to the residue, and the aqueous portion extracted with EtOAc (100 mL×3). The combined organic layers were concentrated to dryness, and the residue was purified by flash column chromatography on silica gel to give B25 (4.5 g, 90% yield). LC-MS: m/z 345 [M+H]⁺.

### Step 4G. Synthesis of tert-butyl-(3-amino-5-methoxycyclohexyl)carbamate (B26)

To a solution of di-tert-butyl-(5-methoxycyclohexane-1,3-diyl)dicarbamate (B25) (600 mg, 1.74 mmol) in DCM (20 mL) was added HCl/MeOH(4 M) (2 mL), and then the reaction mixture was stirred at rt for 16 hrs. The reaction mixture was concentrated to dryness to afford B26 (180 mg, quant.). LC-MS: m/z 245 [M+H]⁺.

### Step 4H. Synthesis of tert-butyl-(3-((2-bromo-5-nitropyridin-4-yl)amino)-5-methoxycyclohexyl)carbamate (B27)

To a solution of di-tert-butyl-(5-methoxycyclohexane-1,3-diyl)dicarbamate (B26) (600 mg, 2.46 mmol) in EtOH (10 mL) were added DIEA (793 mg, 6.14 mmol) and 2,4-dibromo-5-nitropyridine (831 mg, 2.95 mmol), and the reaction mixture was stirred at 40° C. for 16 hrs. The reaction mixture was then concentrated to dryness, and the residue purified by flash column chromatography on silica gel to give B27 (950 mg, 87% yield). LC-MS: m/z 445 [M+H]⁺.

### Step 4I. Synthesis of tert-butyl ((1R,3S,5S)-3-((2-bromo-5-nitropyridin-4-yl)amino)-5-methoxycyclohexyl)carbamate (B28) and tert-butyl ((1S,3R,5R)-3-((2-bromo-5-nitropyridin-4-yl)amino)-5-methoxycyclohexyl)carbamate (B29)

Tert-butyl-(3-((2-bromo-5-nitropyridin-4-yl)amino)-5-methoxycyclohexyl)carbamate (B27) (2.0 g) was separated by chiral SFC to give Enantiomer #1 (772 mg, 39% yield, Rt=0.796 min) and Enantiomer #2 (977 mg, 49% yield, Rt=1.048 min). SFC method: (column: ChiralPak IH (250*30 mm, 5 μm), mobile phase: [0.1% NH₃H₂O MeOH]; %:20%-20%, min).

Enantiomer #1 (B28), tert-butyl ((1R,3S,5S)-3-((2-bromo-5-nitropyridin-4-yl)amino)-5-methoxycyclohexyl)carbamate: LC-MS: m/z 445 [M+H]⁺; 97% ee.

Enantiomer #2 (B29), tert-butyl ((1S,3R,5R)-3-((2-bromo-5-nitropyridin-4-yl)amino)-5-methoxycyclohexyl)carbamate: LC-MS: m/z 445 [M+H]⁺; 98% ee.

### Intermediate Scheme 5: Preparation of tert-butyl (3-amino-2-hydroxycycloheptyl)carbamate (relative stereochemistry is indicated in Intermediate Scheme 5)

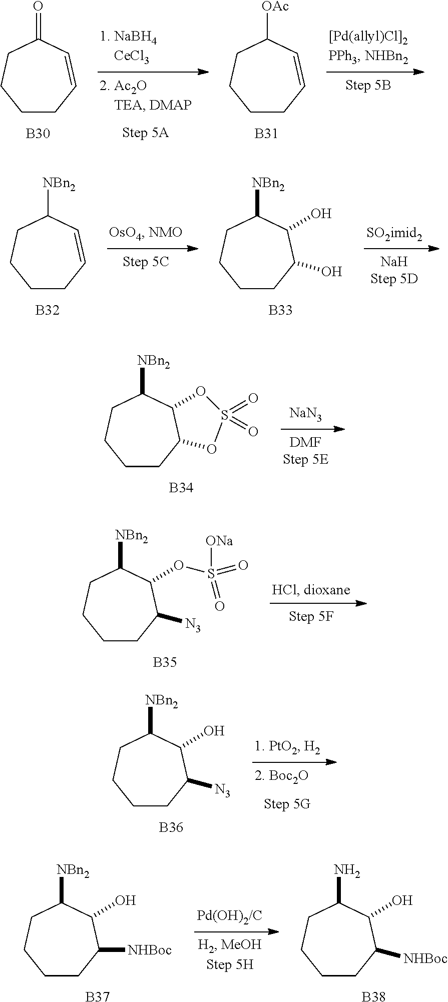

### Step 5A. Synthesis of cyclohept-2-en-1-yl acetate (B31)

To a solution of CeCl₃.7H₂O (33.8 g, 90.8 mmol) in MeOH (91 mL) was added 2-cyclohepten-1-one (B30) (11.1 g, 90.8 mmol) and stirred at 0° C. NaBH₄ (3.47 g, 90.8 mmol) was then added into the reaction mixture in portions, and the reaction mixture was stirred at rt for 16 hrs. 1 M HCl in water (100 mL) was added into the reaction mixture, and the aqueous portion extracted with EtOAc (3×40 mL). The combined organic layers were washed with brine (30 mL), dried over Na₂SO₄, filtered, and concentrated. The resulting suspension was filtered and washed with DCM, and the filtrate was concentrated to give cyclohept-2-en-1-ol (10.2 g, quant.).

The cyclohept-2-en-1-ol (10.2 g, 90.8 mmol) was dissolved in DCM (100 mL), triethylamine (25.5 mL, 182 mmol) was added, and the reaction mixture was cooled to 0° C. Ac₂O (8.59 mL, 90.9 mmol) was added slowly, followed by DMAP (0.57 g, 4.55 mmol) and the reaction mixture warmed to rt. Sat. aq. NaHCO₃ solution (100 mL) was added into the reaction mixture at rt. The organic layer was washed with brine (60 mL), dried over Na₂SO₄, filtered and concentrated to dryness. The residue was purified with a silica pad to give B31 (12.9 g, 92% yield). ¹H NMR (400 MHz, CDCl₃) δ 5.85-5.78 (m, 1H), 5.65-5.61 (m, 1H), 5.40 (d, 1H), 2.24-2.16 (m, 1H), 2.12-2.07 (m, 1H), 2.05 (s, 3H), 1.96-1.81 (m, 2H), 1.72-1.59 (m, 3H), 1.44-1.35 (m, 1H).

### Step 5B. Synthesis of N,N-dibenzylcyclohep-2-en-1-amine (B32)

[Pd(allyl)₂Cl]₂ (2.13 g, 5.7 mmol) and PPh₃ (4.54 g, 17.1 mmol) in anhydrous DCM (285 mL) was degassed with N₂ for 1 hr. Cyclohept-2-en-1-yl acetate (B31) (22.0 g, 143 mmol) and Bn₂NH (56.3 mL, 287 mmol) was added, and the reaction mixture was stirred at rt for 20 hrs. Sat. aq. NH₄Cl solution (250 mL) was added into the reaction mixture at rt, and the resulting mixture was stirred for 1 hr. The organic layer was separated and filtered. The filtrate was washed with brine (150 mL), dried over Na₂SO₄, filtered, and concentrated to dryness. The residue was purified with a silica pad to give B32 (26.6 g, 64% yield). ¹H NMR (400 MHz, CDCl₃) δ 7.43 (d, 4H), 7.35-7.31 (m, 4H), 7.27-7.21 (m, 2H), 5.98 (d, 1H), 5.88-5.82 (m, 1H), 3.75 (d, 2H), 3.60 (d, 2H), 3.37 (d, 1H), 2.21-2.16 (m, 1H), 2.08-1.91 (m, 3H), 1.72-1.63 (m, 1H), 1.58-1.27 (m, 3H); LC-MS: m/z 292.2 [M+H]⁺.

### Step 5C. Synthesis of 3-(dibenzylamino)cycloheptane-1,2-diol (B33)

To a solution of N,N-dibenzylcyclohep-2-en-1-amine (B32) (25.0 g, 85.8 mmol) in 10:1 Acetone/Water (185 mL) was added OsO₄ (4% solution in water, 10 mL, 1.57 mmol) and NMO (50% solution in water, 23.1 mL, 112 mmol), and the reaction mixture stirred at rt. After 16 hrs, additional OsO₄ (4% solution in water, 5 mL, 0.79 mmol) was added, and the reaction mixture stirred for an additional 24 hrs. Sat. aq. Na₂SO₃ solution (150 mL) was added into the reaction mixture, and the aqueous portion was extracted with EtOAc (3×50 mL). The combined organic layers were washed with brine (100 mL), dried over Na₂SO₄, filtered, and concentrated to dryness. The residue was purified by column chromatography on silica gel to give B33 (12.7 g, 46% yield, 55% yield based on recovered starting material). ¹H NMR (400 MHz, CDCl₃) δ 7.34-7.30 (m, 4H), 7.22-7.28 (m, 6H), 4.96 (s, 1H), 4.10-4.08 (m, 1H), 3.81 (d, 2H), 3.49 (dd, 1H), 3.36 (d, 2H), 2.85 (td, 1H), 2.43 (s, 1H), 2.05-1.99 (m, 1H), 1.83-1.65 (m, 3H), 1.60-1.31 (m, 4H); LC-MS: m/z 326.2 [M+H]⁺.

### Step 5D. Synthesis of 4-(dibenzylamino)hexahydro-4H-cyclohepta[d][1,3,2]dioxathiole 2,2-dioxide (B34)

To a solution of 3-(dibenzylamino)cycloheptane-1,2-diol (B33) (12.5 g, 38.5 mmol) in dry THF (250 mL) was added sulfonyl diimidazole (11.8 g, 58.5 mmol) at 0° C. A suspension of NaH (3.85 g, 96.3 mmol) in THF (50 mL) was added at 0° C., and the reaction mixture was allowed to warm to rt. After 18 hrs, the reaction mixture was cooled to 0° C. Sat. aq. NH₄Cl solution (100 mL) was slowly added, and the aqueous portion extracted with EtOAc (3×80 mL). The combined organic layers were washed with brine (100 mL), dried over Na₂SO₄, filtered and concentrated to dryness. The residue was purified by column chromatography on silica gel to give B34 (12.0 g, 80% yield). ¹H NMR (400 MHz, CDCl₃) δ 7.41-7.22 (m, 10H), 5.22 (dd, 1H), 4.79 (ddd, 1H), 3.80 (d, 2H), 3.57 (d, 2H), 3.13 (t, 1H), 2.03-2.12 (m, 1H), 2.02-1.79 (m, 4H), 1.31-1.11 (m, 3H); LC-MS: m/z 388.1 [M+H]⁺.

### Step 5E. Synthesis of sodium-2-azido-7-(dibenzylamino)cycloheptyl sulfate (B35)

To a solution of 4-(dibenzylamino)hexahydro-4H-cyclohepta[d][1,3,2]dioxathiole 2,2-dioxide (B34) (12 g, 30.9 mmol) in DMF (250 mL) was added sodium azide (4.01 g, 61.7 mmol). The reaction mixture was stirred at 50° C. for 3 hrs, and then stirred at rt for 16 hrs. Brine (150 mL) was added into the reaction mixture, and the aqueous portion was extracted with EtOAc (3×60 mL). The combined organic layers were dried over Na₂SO₄, filtered, and concentrated to dryness. The residue was purified by column chromatography on silica gel to give B35 (7.3 g, 52% yield). LC-MS: m/z 429.1 [M-Na]—.

### Step 5F. Synthesis of 2-azido-7-(dibenzylamino)cycloheptan-1-ol (B36)

To a solution of sodium-2-azido-7-(dibenzylamino)cycloheptyl sulfate (B35) (7.28 g, 16.1 mmol) in dioxane (86 mL) was added aq. HCl (4 M in water, 10 mL, 40 mmol), and the reaction mixture was stirred at 80° C. for 4 hrs. Sat. aq. NaHCO₃ solution (80 mL) was then added into the reaction mixture at rt, and the aqueous portion extracted with EtOAc (3×30 mL). The combined organic layers were washed with brine (80 mL), dried over Na₂SO₄, filtered, and concentrated to dryness. The residue was purified by column chromatography on silica gel to give B36 (5.38 g, 95% yield). ¹H NMR (400 MHz, CDCl₃) δ 7.35-7.24 (m, 10H), 4.94 (s, 1H), 3.83 (d, 2H), 3.50 (dd, 1H), 3.31 (d, 2H), 3.17-3.12 (m, 1H), 2.50 (t, 1H), 2.03-1.99 (m, 1H), 1.83-1.63 (m, 3H), 1.58-1.30 (m, 4H); LC-MS: m/z 351.2 [M+H]⁺.

### Step 5G. Synthesis of tert-butyl (3-(dibenzylamino)-2-hydroxycycloheptyl)carbamate (B37)

To a solution of 2-azido-7-(dibenzylamino)cycloheptan-1-ol (B36) (5.35 g, 15.3 mmol) in THF (90 mL) was added PtO₂ (0.89 g). The reaction mixture was degassed under vacuum and purged with H₂ several times and then stirred under H₂ (1 atm) at rt for 5 hrs. The reaction mixture was filtered through celite, and the filtrate was concentrated to −50 mL volume.

To the solution was added Boc₂O (6.34 g, 29.1 mmol), followed by triethylamine (2.6 mL, 18.4 mmol), and stirred at rt for 2 hrs. Brine (50 mL) was added into the reaction mixture, and the aqueous portion was extracted with EtOAc (3×30 mL). The combined organic layers were dried over Na₂SO₄, filtered, and concentrated to dryness. The residue was purified by column chromatography on silica gel to give B37 (4.90 g, 75% yield). ¹H NMR (400 MHz, CDCl₃) δ 7.34-7.22 (m, 10H), 4.90 (s, 1H), 4.78 (s, 1H), 3.81 (d, 2H), 3.35-3.28 (m, 3H), 3.17-3.09 (m, 1H), 2.51 (t, 1H), 2.02-1.89 (m, 2H), 1.82-1.70 (m, 1H), 1.68-1.59 (m, 1H), 1.59-1.32 (m, 4H), 1.41 (s, 9H); LC-MS: m/z 425.2 [M+H]⁺.

### Step 5H. Synthesis of (tert-butyl (3-amino-2-hydroxycycloheptyl)carbamate (B38)

To a solution of tert-butyl (3-(dibenzylamino)-2-hydroxycycloheptyl)carbamate (B37) (4.0 g, 9.42 mmol) in MeOH (35 mL) was added Pd(OH)₂/C (0.47 g). The reaction mixture was degassed under vacuum and purged with H₂ several times. The reaction mixture was stirred under H₂ (1 atm) at rt for 16 hrs. The reaction mixture was filtered, and the filtrate was concentrated to dryness to give B68 (2.22 g, 96% yield). ¹H NMR (400 MHz, CDCl₃) δ 4.78 (s, 1H), 3.50-3.39 (m, 1H), 3.01 (dd, 1H), 2.67 (dt, 1H), 2.60-1.70 (br s, 3H), 1.91-1.87 (m, 1H), 1.80-1.71 (m, 1H), 1.70-1.35 (m, 6H), 1.45 (s, 9H); LC-MS: m/z 245.2 [M+H]⁺.

### Intermediate Scheme 6: Synthesis of 1-methylpiperidine-3,5-diamine (B47)

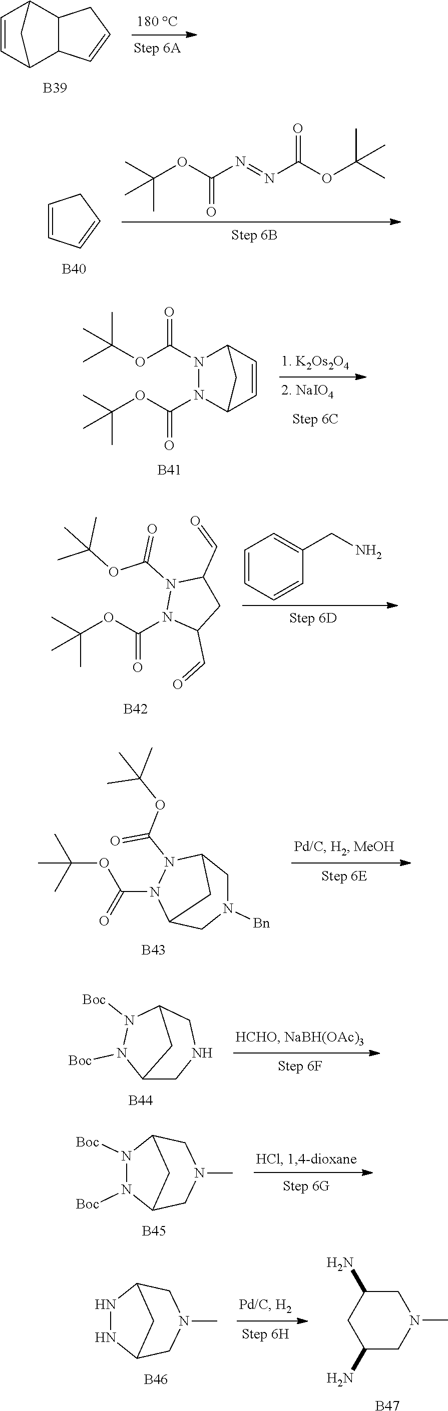

### Step 6A. Synthesis of cyclopenta-1,3-diene (B40)

Tricyclodeca-2,4-diene (B39) (50 g, 378 mmol) was distilled at 180° C. for 5 hrs to give B40 (38.1 g, 76.2% yield). ¹H NMR (400 MHz, CDCl₃) δ 6.58 (d, 2H), 6.47 (t, 2H), 2.98 (s, 2H).

### Step 6B. Synthesis of di-tert-butyl 2,3-diazabicyclo[2.2.1]hept-5-ene-2,3-dicarboxylate (B41)

A solution of di-tert-butyl (E)-diazene-1,2-dicarboxylate (5 g, 21.7 mmol) in DCM (50 mL) was cooled to 0° C. Cyclopenta-1,3-diene (B40) (7.18 g, 109 mmol) was then added. The reaction mixture was stirred at 0° C. for 4 hrs, and then allowed to warm to rt for 12 hrs. The reaction mixture was concentrated to dryness. The residue was purified by column chromatography on silica gel to give B41 (6.3 g, 97.9% yield). LC-MS: m/z 319.3 [M+Na]⁺.

### Step 6C. Synthesis of di-tert-butyl 3,5-diformylpyrazolidine-1,2-dicarboxylate (B42)

To a solution of di-tert-butyl 2,3-diazabicyclo[2.2.1]hept-5-ene-2,3-dicarboxylate (B41) (1.0 g, 3.37 mmol) and K₂OsO₄.2H₂O (124 mg, 337 μmol) in THF (30 mL) was added a solution of NaIO₄ (3.61 g, 16.9 mmol, 935 μL) in H₂O (15 mL). After the addition was complete, the reaction mixture was stirred at rt for 16 hrs. Aq. sat. Na₂SO₃ solution (50 mL) was added, and the aqueous portion extracted with EtOAc (3×30 mL). The combined organic layers were washed with brine (35 mL), dried over Na₂SO₄, filtered, and concentrated to dryness. The residue was purified by column chromatography on silica gel to give B42 (0.57 g, 51.5% yield). ¹H NMR (400 MHz, CDCl₃) δ 9.65 (s, 2H), 4.72 (t, 2H), 2.61 (t, 2H), 1.50 (s, 18H).

### Step 6D. Synthesis of di-tert-butyl 3-benzyl-3,6,7-triazabicyclo[3.2.1]octane-6,7-dicarboxylate (B43)

A solution of di-tert-butyl 3,5-diformylpyrazolidine-1,2-dicarboxylate (B42) (570 mg, 1.74 mmol), phenylmethanamine (186 mg, 1.74 mmol, 189 μL) and one drop of AcOH (10 μL) in DCE (5 mL) was stirred at rt for 1 hr. Then NaBH(OAc)₃ (1.47 g, 6.94 mmol) was added, and the reaction mixture was stirred at rt for another 16 hrs. Water (20 mL) was added, and the aqueous portion was extracted with DCM (3×20 mL). The combined organic layers were washed with brine (30 mL), dried over Na₂SO₄, filtered, and concentrated to dryness. The residue was purified by column chromatography on silica gel to give B43 (0.3 g, 39.9% yield). ¹H NMR (400 MHz, CDCl₃) δ 7.32-7.25 (m, 5H), 4.46-4.32 (m, 3H), 3.68-3.50 (m, 3H), 3.25-3.09 (m, 2H), 2.14-1.91 (m, 2H) 1.51 (s, 18H); LC-MS: m/z 404.3 [M+H]⁺.

### Step 6E. Synthesis of di-tert-butyl 3,6,7-triazabicyclo[3.2.1]octane-6,7-dicarboxylate (B44)

To a solution of di-tert-butyl 3-benzyl-3,6,7-triazabicyclo[3.2.1]octane-6,7-dicarboxylate (B43) (100 mg, 248 μmol) in MeOH (2 mL) was added Pd/C (0.02 g, 10% Pd) under N₂. The suspension was degassed under vacuum and purged with H₂ several times. The reaction mixture was stirred under H₂ (15 psi) at rt for 16 hrs, and then filtered. The filtrate was concentrated to dryness to give B44 (80 mg). LC-MS: m/z 314.3 [M+H]⁺.

### Step 6F. Synthesis of di-tert-butyl 3-methyl-3,6,7-triazabicyclo[3.2.1]octane-6,7-dicarboxylate (B45)

To a solution of di-tert-butyl 3,6,7-triazabicyclo[3.2.1]octane-6,7-dicarboxylate (B44) (0.2 g, 638 μmol) and one drop of AcOH (10 μL) in DCE (5 mL) was added formaldehyde (77.7 mg, 957 μmol, 71.3 μL) (37% in H₂O). After the addition was complete, the reaction mixture was stirred at rt for 1 hr. Then NaBH(OAc)₃ (271 mg, 1.28 mmol) was added, and the reaction mixture was stirred at rt for another 16 hrs. The reaction mixture was concentrated to dryness, and the residue was purified by column chromatography on silica gel to give B45 (0.2 g, 87.7% yield). ¹H NMR (400 MHz, CD₃OD) δ 4.40-4.15 (m, 2H), 3.20-2.95 (m, 2H), 2.23 (s, 3H), 2.17-1.80 (m, 4H), 1.47 (s, 18H); LC-MS: m/z 328.2 [M+H]⁺.

### Step 6G. Synthesis of 3-methyl-3,6,7-triazabicyclo[3.2.1]octane (B46)

A solution of di-tert-butyl 3-methyl-3,6,7-triazabicyclo[3.2.1]octane-6,7-dicarboxylate (B45) (0.2 g, 611 μmol) in HCl (4 M, 6.78 mL) (4M in 1,4-dioxane) was stirred at rt for 1 hr. The reaction mixture was concentrated to dryness to give B46 (0.15 g).

### Step 6H. Synthesis of 1-methylpiperidine-3,5-diamine (B47) (relative stereochemistry at centers 3 and 5 of piperidine ring is cis)

To a solution of 3-methyl-3,6,7-triazabicyclo[3.2.1]octane (B46) (150 mg, 750 μmol, 2HCl) in MeOH (2 mL) was added Pd/C (0.01 g, 10% Pd) under N₂. The suspension was degassed under vacuum and purged with H₂ several times. The mixture was stirred under H₂ (50 psi) at 50° C. for 16 hrs and then cooled to rt. The reaction mixture was filtered, and the filtrate was concentrated to dryness to give B47 (0.14 g, 92.4% yield, 2HCl salt). ¹H NMR (400 MHz, CD₃OD) δ 8.46 (m, 5H), 3.17-3.16 (m, 2H), 2.99 (d, 2H), 2.39-2.27 (m, 1H), 2.26 (s, 3H), 1.90 (t, 2H), 1.45-1.42 (m, 1H); LC-MS: m/z 130.3 [M+H]⁺.

### Intermediate Scheme 7: Synthesis of 1-(3,5-diaminopiperidin-1-yl)ethan-1-one (B50)

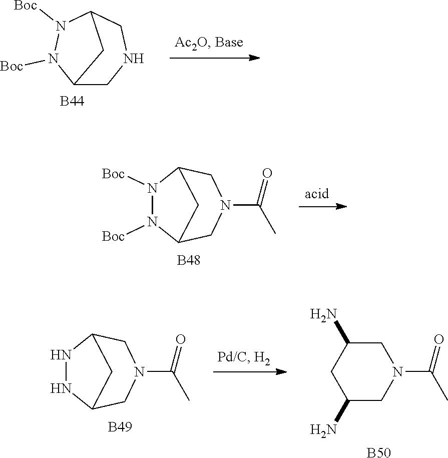

1-(3,5-diaminopiperidin-1-yl)ethan-1-one (B50) (relative stereochemistry at centers 3 and 5 of piperidine ring is cis) may be prepared in accordance with the above scheme by first reacting B44 with acetic anhydride and a base such as TEA to afford di-tert-butyl 3-acetyl-3,6,7-triazabicyclo[3.2.1]octane-6,7-dicarboxylate (B48). The Boc-protecting groups can then be removed by subjecting B48 to acidic conditions such as HCL in 1,4-dioxane to afford 1-(3,6,7-triazabicyclo[3.2.1]octan-3-yl)ethan-1-one (B49). B49 can then be reacted with Pd/C and H₂ gas to afford B50.

### Intermediate Scheme 8: Synthesis of alkyl 3,5-diaminopiperidine-1-carboxylate (B53)

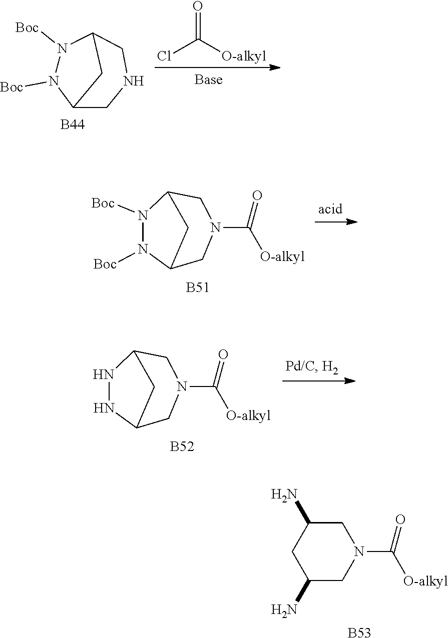

Alkyl 3,5-diaminopiperidine-1-carboxylates (B53) (relative stereochemistry at centers 3 and 5 of piperidine ring is cis) may be prepared in accordance with the above scheme by first reacting B44 with an alkyl chloroformate such as methyl chloroformate and a base such as TEA to afford a 6,7-di-tert-butyl-3-alkyl 3,6,7-triazabicyclo[3.2.1]octane-3,6,7-tricarboxylate (B51). The Boc-protecting groups can then be removed by subjecting B51 to acidic conditions such as HCl in 1,4-dioxane to afford an alkyl 3,6,7-triazabicyclo[3.2.1]octan-3-carboxylate (B52). B52 can then be reacted with Pd/C and H₂ gas to afford B53.

### Intermediate Scheme 9: Synthesis of 1-(alkylsulfonyl)piperidine-3,5-diamines (B56)

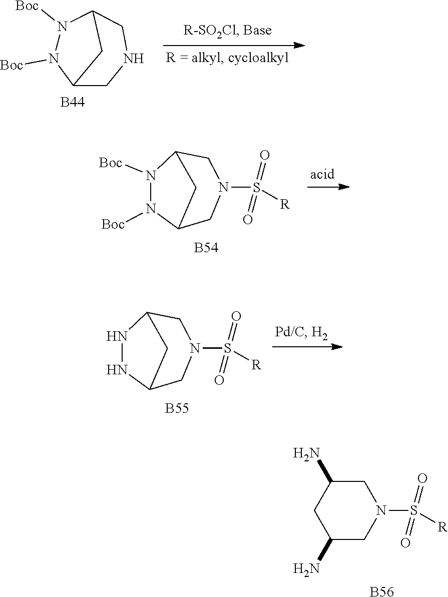

1-(alkylsulfonyl)piperidine-3,5-diamines and 1-cycloalkylsulfonyl)piperidine-3,5-diamines (B56) (relative stereochemistry at centers 3 and 5 of piperidine ring is cis) may be prepared in accordance with the above scheme by first reacting B44 with alkylsulfonylchloride or cycloalkylsulfonylchloride and a base such as TEA to afford di-tert-butyl 3-(alkylsulfonyl)-3,6,7-triazabicyclo[3.2.1]octane-6,7-dicarboxylate or di-tert-butyl 3-(cycloalkylsulfonyl)-3,6,7-triazabicyclo[3.2.1]octane-6,7-dicarboxylate (B54). The Boc-protecting groups can then be removed by subjecting B54 to acidic conditions such as HCl in 1,4-dioxane to afford 3-(alkylsulfonyl)-3,6,7-triazabicyclo[3.2.1]octane or 3-(cycloalkylsulfonyl)-3,6,7-triazabicyclo[3.2.1]octane (B55). B55 can then be reacted with Pd/C and H₂ gas to afford B56.

### Intermediate Scheme 10: Preparation of tert-butyl (5-((5-bromo-2-nitrophenyl)amino)-3,3-difluorocyclohexyl)carbamate (relative stereochemistry is indicated in Intermediate Scheme 10)

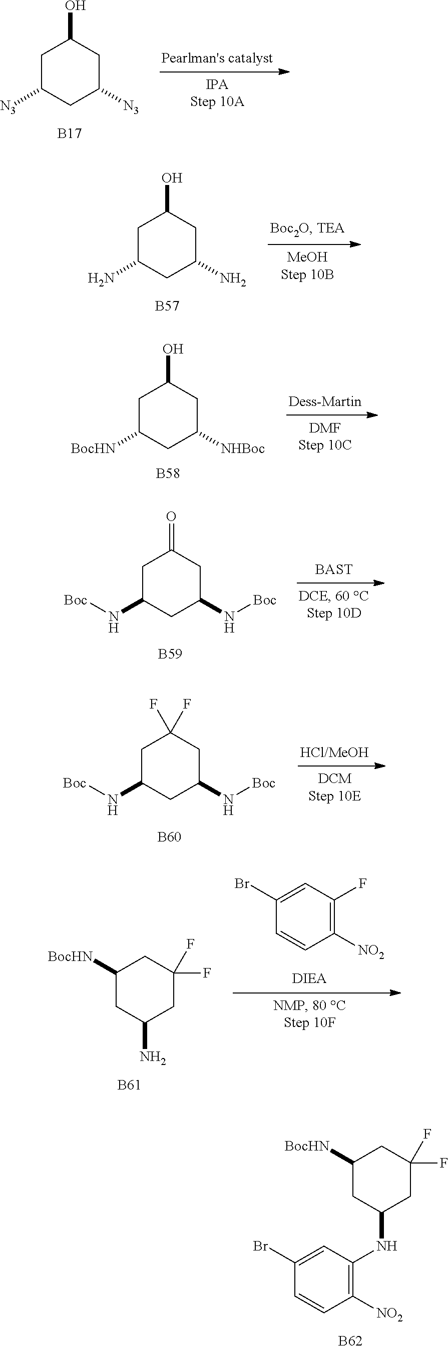

### Step 10A. Synthesis of 3,5-diaminocyclohexan-1-ol (B57)

To a solution of 3,5-diazidocyclohexan-1-ol (B17) (5 g, 27.4 mmol) in isopropanol (30 mL) was added Pearlman's catalyst (2.92 g, 27.4 mmol) under nitrogen. The reaction mixture was degassed under vacuum and purged with H₂ several times and then stirred at rt for 12 hrs. The reaction mixture was filtered through a pad of Celite®, and the filter cake was washed with MeOH (20 mL). The filtrates were concentrated to dryness to give B57 (3.3 g, 92% yield). LCMS: m/z 131 [M+H]⁺.

### Step 10B. Synthesis of di-tert-butyl (5-hydroxycyclohexane-1,3-diyl)dicarbamate (B58)

To a solution of 3,5-diaminocyclohexan-1-ol (B57) (2.8 g, 21.5 mmol) in MeOH (30 mL) were added TEA (10.9 g, 107 mmol) and di-tert-butyl dicarbonate (18.8 g, 86.0 mmol), and the reaction mixture was stirred at rt for 12 hrs. After completion, the reaction mixture was washed with aq. 1N HCl solution (20 mL), and the aqueous portion extracted with EtOAc (60 mL×3). The combined organic layers were washed with brine (50 mL), dried over Na₂SO₄, filtered, and concentrated to dryness. The residue was purified by flash column chromatography on silica gel to afford B58 (6.3 g, 89% yield). LC-MS: m/z 331 [M+H]⁺.

### Step 10C. Synthesis of di-tert-butyl (5-oxocyclohexane-1,3-diyl)dicarbamate (B59)

To a solution of di-tert-butyl (5-hydroxycyclohexane-1,3-diyl)dicarbamate (B58) (5.8 g, 17.6 mmol) in DMF (100 mL) was added Dess-Martin periodinane (14.9 g, 35.1 mmol), and the reaction mixture was stirred at rt for 2 hrs. After completion, the reaction mixture was quenched by the addition of sat. aq. NaHCO₃ solution (20 mL) and sat. aq. Na₂S₂O₃ solution (20 mL), and the aqueous portion extracted with EtOAc (100 mL×3). The combined organic layers were washed with brine (100 mL), dried over Na₂SO₄, filtered, and concentrated to dryness. The residue was purified by flash column chromatography on silica gel to afford B59 (5.1 g, 88% yield). LC-MS: m/z 329 [M+H]⁺.

### Step 10D. Synthesis of di-tert-butyl (5,5-difluorocyclohexane-1,3-diyl)dicarbamate (B60)

To a solution of di-tert-butyl (5-oxocyclohexane-1,3-diyl)dicarbamate (B59) (5.1 g, 15.5 mmol) in DCE (100 mL) was added BAST (27.5 g, 124 mmol). The reaction mixture was stirred at 60° C. for 12 hrs. After completion, the reaction mixture was cooled to rt and then quenched by the addition of sat. aq. NaHCO₃ solution (50 mL). The aqueous portion was extracted with DCM (100 mL×3), and the combined organic layers were washed with brine (100 mL), dried over Na₂SO₄, filtered, and concentrated to dryness. The residue was purified by flash column chromatography on silica gel to afford B60 (850 mg, 16% yield). LC-MS: m/z 351 [M+H]⁺.

### Step 10E. Synthesis of tert-butyl (5-amino-3,3-difluorocyclohexyl)carbamate (B61)

To a solution of di-tert-butyl (5,5-difluorocyclohexane-1,3-diyl)dicarbamate (B60) (500 mg, 1.43 mmol) in DCM (10 mL) was added HCl/MeOH (0.5 mL, 2.15 mmol, 4 M). The reaction mixture was stirred at rt for 12 hrs. After completion, water (20 mL) was added to the reaction mixture, and the aqueous portion extracted with EtOAc (20 mL×2). Sat. aq. NaHCO₃ solution was added to the aqueous portion until the pH was 9. The aqueous portion was extracted with DCM (20 mL×3), and the combined organic layers were washed with brine (10 mL), dried over Na₂SO₄, filtered, and concentrated to dryness to give B61 (300 mg, quant.), which was used without further purification. LCMS: m/z 251 [M+H]⁺.

### Step 10F. Synthesis of tert-butyl (5-((5-bromo-2-nitrophenyl)amino)-3,3-difluorocyclohexyl)carbamate (B62)

To a solution of tert-butyl (5-amino-3,3-difluorocyclohexyl)carbamate (B61) (554 mg, 2.52 mmol) and 4-bromo-2-fluoro-1-nitrobenzene (554 mg, 2.52 mmol) in NMP (10 mL) was added DIEA (651 mg, 5.03 mmol). The reaction mixture was stirred at 80° C. for 1 hr under N₂ atmosphere. The reaction mixture was cooled to rt, and water (10 mL) was added. The aqueous portion was extracted with EtOAc (20 mL×3), and the combined organic layers were washed with brine (15 mL), dried over Na₂SO₄, filtered, and concentrated to dryness. The residue was purified by flash column chromatography on silica gel to give B62 (561 mg, 50% yield). LC-MS: m/z 450 [M+H]⁺.

Intermediate tert-butyl (5-((2-bromo-5-nitropyridin-4-yl)amino)-3,3-difluorocyclohexyl)carbamate

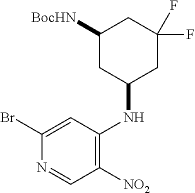

may be prepared by following the procedure in Steps 10A to 10E of Intermediate Scheme 10 and then reacting tert-butyl (5-amino-3,3-difluorocyclohexyl)carbamate (B61) with 2,4-dibromo-5-nitropyridine in Step 10F.

### Intermediate Scheme 11: Synthesis of (relative stereochemistry is indicated in Intermediate Scheme 11)

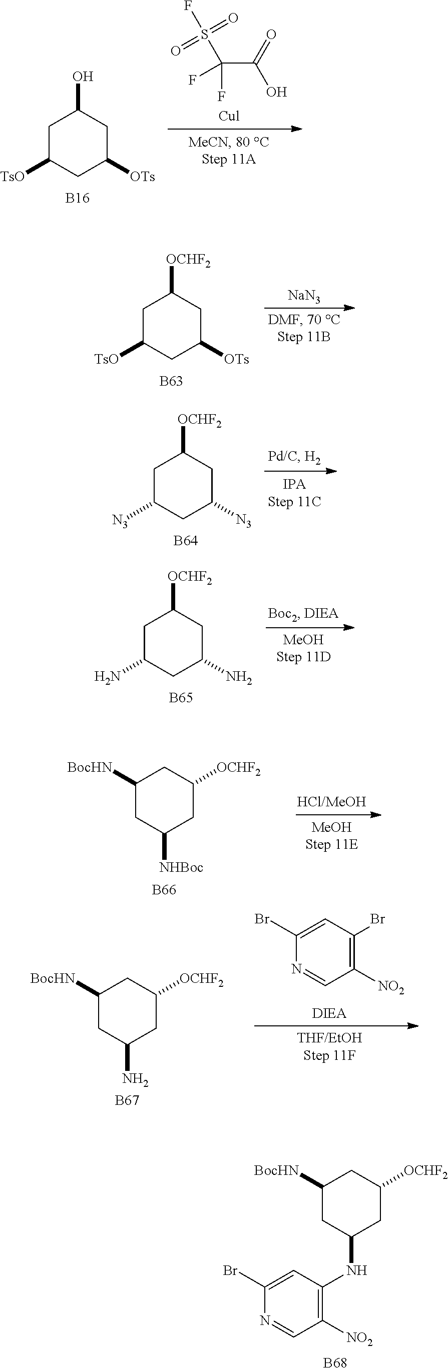

### Step 11A. Synthesis of 5-(difluoromethoxy)cyclohexane-1,3-diyl bis(4-methylbenzenesulfonate) (B63)

To a solution of 5-hydroxycyclohexane-1,3-diyl bis(4-methylbenzenesulfonate) (B16) (7.6 g, 17 mmol) and CuI (0.66 g, 3.5 mmol) in MeCN (100 mL) was added 2,2-difluoro-2-(fluorosulfonyl)acetic acid (6.1 g, 35 mmol). The reaction mixture was stirred at 80° C. for 2 hrs and then concentrated to dryness. The residue was purified by flash column chromatography on silica gel to give B63 (5.1 g, 60% yield). LC-MS: m/z 491 [M+H]⁺.

### Step 11B. Synthesis of 1,3-diazido-5-(difluoromethoxy)cyclohexane (B64)

To a solution of 5-(difluoromethoxy)cyclohexane-1,3-diyl bis(4-methylbenzenesulfonate) (B63) (5.3 g, 11 mmol) in DMF (60 mL) was added sodium azide (2.1 g, 32 mmol) at rt. The reaction mixture was then stirred at 70° C. for 15 hrs. The reaction mixture was cooled to rt, and sat. aq. NH₄Cl solution was added (50 mL). The aqueous portion was extracted with EtOAc (60 mL×3), and the combined organic layers were washed with brine (50 mL), dried over Na₂SO₄, filtered, and concentrated to dryness. The residue was purified by flash column chromatography on silica gel to give B64 (2.4 g, 96% yield). LC-MS: m/z 233 [M+H]⁺.

### Step 11C. Synthesis of 5-(difluoromethoxy)cyclohexane-1,3-diamine (B65)

To the solution of 1,3-diazido-5-(difluoromethoxy)cyclohexane (B64) (2.3 g, 9.9 mmol) in i-PrOH (30 mL) was added Pd/C (500 mg, 9.9 mmol) under N₂ atmosphere. The reaction mixture was degassed and purged with H₂ several times, and then heated at 25° C. for additional 15 hrs under H₂ atmosphere. The reaction mixture was filtered through a Celite pad, and the filter cake was washed with MeOH (20 mL). The filtrate was concentrated to dryness to give B65 (1.7 g, quant.), which was used without further purification. LC-MS: m/z 181 [M+H]⁺.

### Step 11D. Synthesis of di-tert-butyl (5-(difluoromethoxy)cyclohexane-1,3-diyl)dicarbamate (B66)

To a solution of 5-(difluoromethoxy)cyclohexane-1,3-diamine (B65) (1.4 g, 7.8 mmol) in MeOH (25 mL) were added DIEA (4.0 g, 31 mmol) and di-tert-butyl dicarbonate (4.2 g, 19 mmol), and the reaction mixture was stirred at rt for 15 hrs. After completion, the reaction mixture was filtered, and the wet cake was washed with MeOH (20 mL). The wet cake was dried to give B66 (1.7 g, 58% yield). LC-MS: m/z 381 [M+H]⁺.

### Step 11E. Synthesis of tert-butyl (3-amino-5-(difluoromethoxy)cyclohexyl) carbamate (B67)

To a solution of di-tert-butyl (5-(difluoromethoxy)cyclohexane-1,3-diyl)dicarbamate (B66) (2.0 g, 5.3 mmol) in MeOH (25 mL) was added HCl/MeOH (1.2 g, 16 wt %, 5.3 mmol). Then the reaction mixture was stirred at rt for 15 hrs. After completion, the reaction mixture was poured into H₂O (20 mL), and the aqueous portion extracted with EtOAc (40 mL×2). Sat. aq. NaHCO₃ solution was added slowly until the pH was equal to 9. The aqueous portion was then extracted with DCM/MeOH (50 mL×3), and the combined organic layers were washed with brine (20 mL), dried with Na₂SO₄, filtered, and concentrated to dryness to give B67 (400 mg, 27% yield), which was used without further purification. LCMS: m/z 281 [M+H]⁺.

### Step 11F. Synthesis of tert-butyl (3-((2-bromo-5-nitropyridin-4-yl)amino)-5-(difluoromethoxy)cyclohexyl)carbamate (B68)

To a solution of tert-butyl ((1R,3S,5R)-3-amino-5-(difluoromethoxy)cyclohexyl)carbamate (B67) (450 mg, 1.61 mmol) and 2,4-dibromo-5-nitropyridine (498 mg, 1.77 mmol) in THF/EtOH (5 mL/5 mL) was added DIEA (622 mg, 4.82 mmol), and the reaction mixture was stirred at rt for 15 hrs under N₂ atmosphere. Water (15 mL) was added, and the aqueous portion extracted with EtOAc (20 mL×3). The combined organic layers were washed with brine (15 mL), dried over Na₂SO₄, filtered, and concentrated to dryness. The residue was purified by flash column chromatography on silica gel to give B68 (580 mg, 75% yield). LC-MS: m/z 481 [M+H]⁺.

### Intermediate Scheme 12: Preparation of 3,5-diaminotetrahydro-2H-thiopyran 1,1-dioxide (relative stereochemistry is indicated in Intermediate Scheme 12 for Steps 12H, 12I, and 12J)

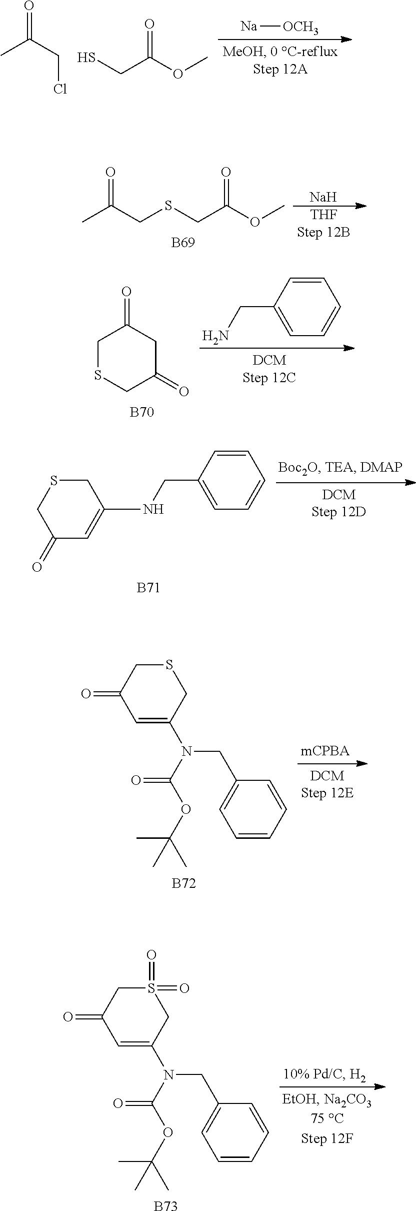

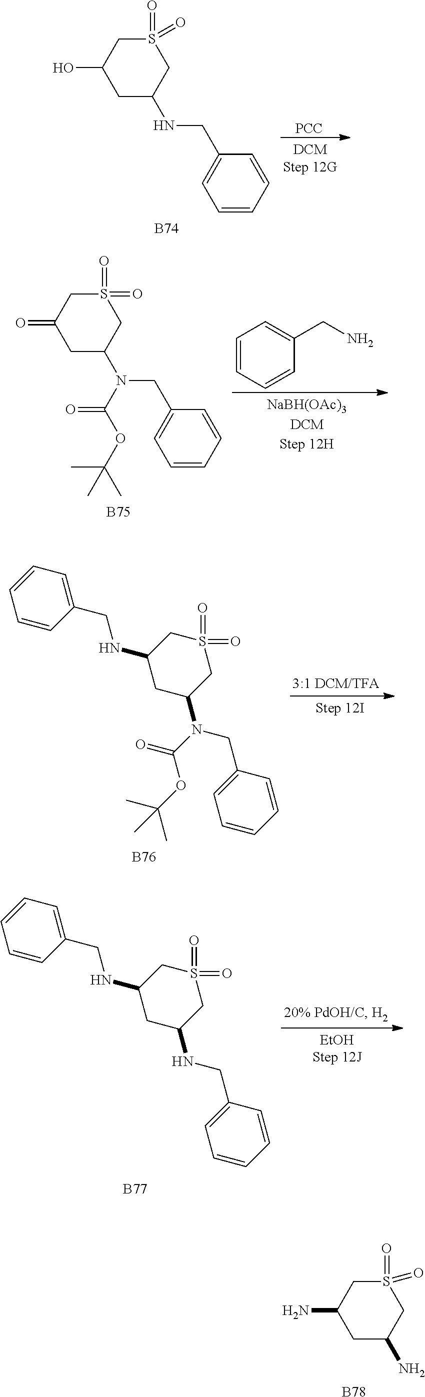

### Step 12A. Synthesis of methyl 2-((2-oxopropyl)thio)acetate (B69)

A solution of 300 mL of anhydrous methanol and 10.18 g of sodium methoxide (190 mmol) was cooled in an ice bath and then 16.9 mL of methyl thioglycolate (190 mmol) was added. After 5 minutes of stirring, to the reaction mixture was slowly added 17.4 g of chloroacetone (15 mL, 190 mmol). Upon completion of chloroacetone addition, the reaction mixture was warmed to rt for 15 minutes and then heated to 70° C. for 1 hr. The reaction mixture was cooled, filtered, and concentrated. MTBE was added, followed by water. The aqueous layer was extracted 3 times with MTBE, and the combined organic layers were washed with sat. sodium bicarbonate solution, dried over Na₂SO₄, filtered, and concentrated to dryness to afford 26.72 g of B69, which was used without further purification. ¹H NMR (400 MHz, CDCl₃) δ 3.73 (d, 3H), 3.42 (s, 2H), 3.26 (s, 2H), 2.28 (s, 3H).

### Step 12B. Synthesis of 2H-thiopyran-3,5(4H,6H)-dione (B70)

A solution of 8.33 g of sodium hydride (60% dispersion in oil, 210 mmol) in 100 mL of anhydrous THF was cooled in an ice water bath and then 26.72 g (165 mmol) of methyl 2-((2-oxopropyl)thio)acetate (B69) as a solution in 105 mL of THF was slowly added to the reaction mixture via cannula. The reaction mixture was stirred for several hours. Water (150 mL) was added, and the aqueous portion extracted several times with MTBE. The aqueous layer was acidified to a pH between 1.5-2.5 and extracted several times with DCM. The combined organic layers were dried over Na₂SO₄, filtered, and concentrated to dryness. The residue was dissolved in minimal MTBE with minimal DCM and placed in the −20° C. fridge for two days and then the material was washed once again with MTBE containing heptanes. The material was decanted and then dried to afford 12.73 grams of B70, which was used without further purification (59.4% yield). LC-MS: m/z 131.0 [M+H]⁺.

### Step 12C. Synthesis of 5-(benzylamino)-2H-thiopyran-3(6H)-one (B71)

A solution of 14.05 g of 2H-thiopyran-3,5(4H,6H)-dione (B70) (108 mmol) in 300 mL of DCM was cooled in an ice bath and then 11.8 mL of benzyl amine (108 mmol) were added, and the reaction mixture allowed to stir for 20 hrs. Precipitation was observed during the initial stages of the reaction. After the 20 hrs, the precipitate was separated from the reaction mixture using a fritted filter funnel and washed with DCM several times. The precipitate was dried to afford 10.37 g of B71. The filtrate solution's solvent was concentrated to dryness, and the residue was purified by column chromatography (330 g normal phase isco column; eluent 25-100% EtOAc:heptanes) to afford 3.5 grams of B70 (58.6% total yield from precipitation and column chromatography). ¹H NMR (400 MHz, DMSO-d₆) δ 7.69 (s, 1H), 7.39-7.24 (m, 2H), 4.82 (s, 1H), 4.23 (d, 1H), 3.44 (s, 1H), 3.09 (s, 1H); LC-MS: m/z 220.1 [M+H]⁺.

### Step 12D. Synthesis of tert-butyl benzyl(5-oxo-5,6-dihydro-2H-thiopyran-3-yl)carbamate (B72)

To a solution of 5.94 g of 5-(benzylamino)-2H-thiopyran-3(6H)-one (B71) (27.1 mmol) in 100 mL DCM, 5.7 mL of triethyl amine (4.14 g, 41 mmol), 662 mg of DMAP (5.42 mmol) and 8.57 g of Boc anhydride (39.3 mmol) were added. The flask was flushed with argon, and the reaction mixture allowed to stir overnight at rt and then concentrated to dryness. The residue was purified by column chromatography (normal phase isco column; eluent 0-25% EtOAc:heptanes) to afford 2.86 g of B72 (38.1% yield). LC-MS: m/z 264.1 [M+H⁺-Tbutyl].

### Step 12E. Synthesis of tert-butyl benzyl(1,1-dioxido-5-oxo-5,6-dihydro-2H-thiopyran-3-yl)carbamate (B73)

A solution of 4.29 g of tert-butyl benzyl(5-oxo-5,6-dihydro-2H-thiopyran-3-yl)carbamate (B72) (13.4 mmol) in 300 mL DCM was cooled in an ice water bath. Then, 7.52 g mCPBA (33.6 mmol; 77% purity) was added portion wise. After 15 minutes the reaction mixture was allowed to warm to rt and stirred for an additional 2 hrs. The reaction mixture was quenched by the addition of solid sodium sulfite and solid sodium bicarbonate, and then sat. aq. sodium bicarbonate solution, sat. aq. sodium sulfite, and deionized water were added. The aqueous portion was extracted with DCM (2×), and the combined organic layers dried over Na₂SO₄, filtered, and concentrated to dryness. The residue was purified by column chromatography (80 g normal phase isco gold column; eluent 0-45% EtOAc:heptanes) to afford 4.26 g of B73 (90.3% yield). LC-MS: m/z 350.1 [M−H]⁺.

### Step 12F. Synthesis of tert-butyl benzyl(5-hydroxy-1,1-dioxidotetrahydro-2H-thiopyran-3-yl)carbamate (B74)

9.4 g of tert-butyl benzyl(1,1-dioxido-5-oxo-5,6-dihydro-2H-thiopyran-3-yl)carbamate (B73) (26.7 mmol) and 2.84 g of sodium carbonate (26.8 mmol) were dissolved/suspended in 450 mL of 200 proof EtOH and 5.5 g of 10% Pd/C were added. The reaction mixture was sparged with hydrogen, and the flask flushed with hydrogen gas. The reaction mixture was then heated to 75° C. under a hydrogen atmosphere overnight. The reaction mixture was then filtered, and the filtrate concentrated. The residue was dry loaded onto silica gel, and purified by column chromatography (normal phase isco column, gradient 0-50% ethyl acetate in heptanes) to afford 798 mg of B74 (8.39% yield). LC-MS: m/z 256.1 [M+H⁺-BOC].

### Step 12G. Synthesis of tert-butyl benzyl(1,1-dioxido-5-oxotetrahydro-2H-thiopyran-3-yl)carbamate (B75)

760 mg (2.14 mmol) of tert-butyl benzyl(5-hydroxy-1,1-dioxidotetrahydro-2H-thiopyran-3-yl)carbamate (B74), PCC (553 mg, 2.56 mmol) and 100 mg of crushed 4 Å molecular sieves were dissolved/suspended in 7 mL of dry DCM, and the reaction mixture stirred at rt for 3 hrs. After 2.5 hrs, additional PCC (~0.5 eq) was added and within 30 minutes LC-MS showed full consumption of starting material. The reaction mixture was passed through a plug of silica using 40% ethyl acetate in heptanes to afford 610 mg of B75 (80.7% yield), which was used without further purification. LC-MS: m/z 352.2 [M−H⁺].

### Step 12H. Synthesis of tert-butyl benzyl(5-(benzylamino)-1,1-dioxidotetrahydro-2H-thiopyran-3-yl)carbamate (B76)

To a solution of tert-butyl benzyl(1,1-dioxido-5-oxotetrahydro-2H-thiopyran-3-yl)carbamate (B75) (610 mg, 1.73 mmol) in 9 mL of DCM was added 185 mg (188 μL, 1.73 mmol) of benzyl amine. The reaction mixture was allowed to stir for 1 hr and 45 min, and then sodium triacetoxy borohydride was added (733 mg, 3.46 mmol). The reaction mixture was then allowed to stir overnight. Additional aliquots of benzyl amine and sodium triacetoxy borohydride were added until all the starting material was consumed by LC-MS. After reaction completion, sat. aq. sodium bicarbonate solution was added, and the aqueous portion extracted several times with EtOAc. The combined organic extracts were concentrated to dryness, and the residue held under high vacuum for several hours. The residue was then dissolved in DCM and passed through a silica plug which had been neutralized with Hüing's base (eluent EtOAc) to give 620 mg of B76 (80.8% yield), which was used without further purification. LC-MS: m/z 445.2 [M+H⁺].

### Step 121. Synthesis of 3,5-bis(benzylamino)tetrahydro-2H-thiopyran 1,1-dioxide (B77)

620 mg (1.39 mmol) of tert-butyl benzyl-5-(benzylamino)-1,1-dioxidotetrahydro-2H-thiopyran-3-yl)carbamate (B76) was dissolved in 30 mL of 20% TFA in DCM and allowed to stir at rt until LC-MS showed complete loss of the Boc protecting group. The reaction mixture was then quenched by adding solid sodium carbonate followed by minimal sat. aq. sodium bicarbonate solution. The aqueous portion was extracted with EtOAc, and the organic layer concentrated to dryness. The residue was dissolved in DCM and passed through a silica plug (eluent EtOAc) and concentrated to dryness to afford B77, which was used without further purification. LC-MS: m/z 345.2 [M+H⁺].

### Step 12J. Synthesis of 3,5-diaminotetrahydro-2H-thiopyran 1,1-dioxide (B78)

3,5-bis(benzylamino)tetrahydro-2H-thiopyran 1,1-dioxide (B77) was dissolved in 8 ml of methanol and 250 mg of 20% PdOH/C was added. The reaction mixture was then flushed with hydrogen and kept under an atmosphere of hydrogen via a hydrogen balloon and heated to 80° C. overnight. The reaction mixture was cooled and passed through a celite plug after LC-MS indicated that the hydrogenolysis was complete. The celite was washed with MeOH and EtOAc. The organic portion were concentrated to dryness to afford B78, which was used without further purification. ¹H NMR (400 MHz, CD₃OD) δ 3.58-3.51 (m, 1H), 3.26 (ddd, 1H), 2.97 (ddd, 1H), 1.84 (t, 1H); LC-MS: m/z 165.0 [M+H⁺].

### Intermediate Scheme 13: Preparation of benzyl tert-butyl (5-cyanocyclohexane-1,3-diyl)dicarbamate (relative stereochemistry is indicated in Intermediate Scheme 13)

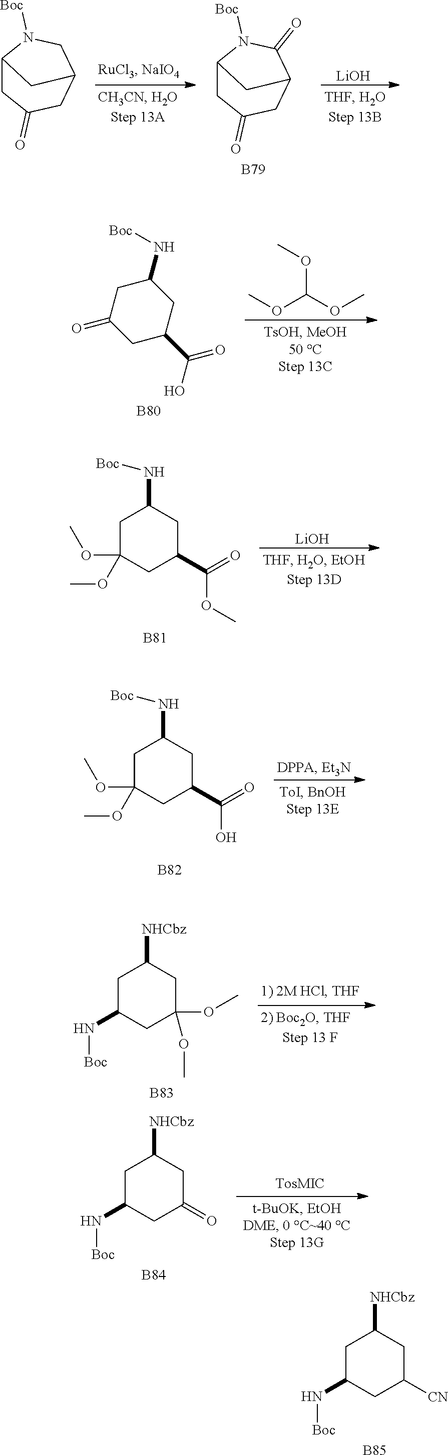

### Step 13A. Synthesis of tert-butyl 3,7-dioxo-6-azabicyclo[3.2.1]octane-6-carboxylate (B79)

To a solution of tert-butyl 3-oxo-6-azabicyclo[3.2.1]octane-6-carboxylate (3.2 g, 14.2 mmol) in EtOAc (20 mL) was added a solution of RuCl₃·H₂O (160 mg, 710 μmol) and NaIO₄ (12.15 g, 56.8 mmol, 3.15 mL) in H₂O (15 mL). The reaction mixture was stirred at rt for 18 hrs. Isopropanol (6 mL) was added, and the reaction mixture was stirred at rt for another 20 min and concentrated to dryness. The residue was dissolved in EtOAc (100 mL), washed with water (100 mL) and brine (100 mL), dried over Na₂SO₄, filtered, and concentrated to dryness. The residue was purified by flash silica gel chromatography to give B79 (2.8 g, 82.4% yield). ¹H NMR (400 MHz, CDCl₃) δ 4.59-4.58 (m, 1H), 2.92-2.90 (m, 2H), 2.78-2.76 (m, 1H), 2.64-2.51 (m, 3H), 1.94-1.91 (m, 1H), 1.53 (s, 9H).

### Step 13B. Synthesis of 3-((tert-butoxycarbonyl)amino)-5-oxocyclohexane-1-carboxylic acid (B80)

To a mixture of tert-butyl 3,7-dioxo-6-azabicyclo[3.2.1]octane-6-carboxylate (B79) (2.8 g, 11.7 mmol) in THF (30 mL)/H₂O (20 mL) was added LiOH·H₂O (1.23 g, 29.3 mmol), and the reaction mixture was stirred at rt for 18 hrs. Water (50 mL) was added to the reaction mixture, and the pH was adjusted to 3 by the addition of 0.5 M HCl. The aqueous portion was extracted with EtOAc (40 mL×3). The combined organic layers were washed with brine (40 mL×2), dried over Na₂SO₄, filtered, and concentrated to dryness to give B80 (3.0 g, 99.6% yield), which was used without further purification. ¹H NMR (400 MHz, CDCl₃) δ 4.62 (br s, 1H), 3.87 (br s, 1H), 2.78-2.73 (m, 2H), 2.64-2.62 (m, 1H), 2.52-2.46 (m, 2H), 2.28-2.23 (m, 1H), 1.46 (s, 9H).

### Step 13C. Synthesis of methyl-5-((tert-butoxycarbonyl)amino)-3,3-dimethoxycyclohexane-1-carboxylate (B81)

A mixture of 3-(tert-butoxycarbonylamino)-5-oxo-cyclohexanecarboxylic acid (B80) (3.0 g, 11.7 mmol), trimethoxymethane (4.95 g, 46.6 mmol, 5.11 mL) and TsOH (201 mg, 1.17 mmol) in MeOH (40 mL) was stirred at 50° C. for 18 hrs. The reaction mixture was concentrated to dryness. The residue was purified by flash silica gel chromatography to give B81 (2.5 g, 67.6% yield). ¹H NMR (400 MHz, CDCl₃) δ 4.41-4.39 (m, 1H), 3.68 (s, 3H), 3.65 (br s, 1H), 3.22 (s, 3H), 3.20 (s, 3H), 2.66-2.61 (m, 1H), 2.37-2.27 (m, 3H), 1.46 (s, 9H), 1.41-1.37 (m, 1H), 1.26-1.09 (m, 2H); LC-MS: m/z 340.1 (M+Na)⁺.

### Step 13D. Synthesis of 5-((tert-butoxycarbonyl)amino)-3,3-dimethoxycyclohexane-1-carboxylic acid (B82)

A mixture of methyl-5-((tert-butoxycarbonyl)amino)-3,3-dimethoxycyclohexane-1-carboxylate (B81) (1.8 g, 5.67 mmol) and LiOH·H₂O (1.19 g, 28.4 mmol) in THF (8 mL)/H₂O (8 mL)/EtOH (2 mL) was stirred at rt for 18 hrs. Water (50 mL) was added to the reaction mixture, and the pH adjusted to 4 by adding aq. citric acid solution. The aqueous portion was extracted with EtOAc (25 mL×3). The combined organic layers were washed with brine (20 mL×3), dried over Na₂SO₄, filtered, and concentrated to dryness to give B82 (1.7 g), which was used without further purification. ¹H NMR (400 MHz, CDCl₃) δ 4.44-4.42 (m, 1H), 3.65 (br s, 1H), 3.23 (s, 3H), 3.20 (s, 3H), 2.68-2.62 (m, 1H), 2.37-2.24 (m, 3H), 1.45 (s, 9H), 1.39-1.21 (m, 3H).

### Step 13E. Synthesis of benzyl tert-butyl (5,5-dimethoxycyclohexane-1,3-diyl)dicarbamate (B83)

To a solution of 5-((tert-butoxycarbonyl)amino)-3,3-dimethoxycyclohexane-1-carboxylic acid (B82) (2.3 g, 7.58 mmol) and Et₃N (1.15 g, 11.4 mmol, 1.58 mL) in toluene (35 mL) was added DPPA (2.71 g, 9.86 mmol, 2.14 mL), and the reaction mixture was stirred at rt for 2 hrs. Then BnOH (1.64 g, 15.2 mmol, 1.58 mL) was added and the reaction mixture was stirred at 100° C. for 18 hrs. The reaction mixture was concentrated to dryness, and the residue was purified by flash silica gel chromatography to give B83 (2.2 g, 71.0% yield). LC-MS: 431.0 (M+Na)⁺.

### Step 13F. Synthesis of benzyl tert-butyl (5-oxocyclohexane-1,3-diyl)dicarbamate (B84)

To a solution of tert-butyl N-[5-(benzyloxycarbonylamino)-3,3-dimethoxy-cyclohexyl]carbamate (B83) (2.2 g, 5.39 mmol) in THF (20 mL) was added HCl (2 M, 8.08 mL). The reaction mixture was stirred at 60° C. for 18 hrs and then cooled to rt. THF (10 mL) was added to the reaction mixture, and pH adjusted to 8 by adding sat. aq. Na₂CO₃ solution. Boc₂O (2.35 g, 10.77 mmol, 2.47 mL) was added, and the reaction mixture was stirred at rt for 2 hrs. Water (50 mL) was added, and the aqueous portion extracted with EtOAc (25 mL×3). The combined organic layers were washed with brine (25 mL), dried over Na₂SO₄, filtered, and concentrated to dryness. The residue was purified by flash silica gel chromatography to give B84 (1.5 g, 76.9% yield). ¹H NMR (400 MHz, CDCl₃) δ 7.41-7.33 (m, 5H), 5.12 (s, 2H), 4.83 (br s, 1H), 4.55 (br s, 1H), 3.89-3.83 (m, 2H), 2.79-2.72 (m, 2H), 2.51-2.48 (m, 1H), 2.28-2.22 (m, 2H), 1.68 (br s, 1H), 1.46 (s, 9H); LC-MS: 484.2 (M+Na)⁺.

### Step 13G. Synthesis of benzyl tert-butyl (5-cyanocyclohexane-1,3-diyl)dicarbamate (B85)

To a solution of benzyl tert-butyl (5-oxocyclohexane-1,3-diyl)dicarbamate (B84) (1.5 g, 4.14 mmol), 1-(isocyanomethylsulfonyl)-4-methyl-benzene (970 mg, 4.97 mmol) and EtOH (229 mg, 4.97 mmol) in DME (10 mL) was added t-BuOK (604 mg, 5.38 mmol) at 0° C., and the reaction mixture was stirred at 0° C. to rt for 1 hr. The reaction mixture was then heated to 40° C. and stirred for 18 hrs. The reaction mixture was then concentrated to dryness to give a residue. The residue was purified by silica gel chromatography to give B85 (370 mg, 23.9% yield). LC-MS: 396.0 (M+Na)⁺.

### Intermediate Scheme 14: Preparation of intermediate rel-tert-butyl ((1R,3S)-3-amino-2-hydroxy-2-methylcyclohexyl)carbamate (relative stereochemistry is indicated in Intermediate Scheme 14 for steps 14A and 14B)

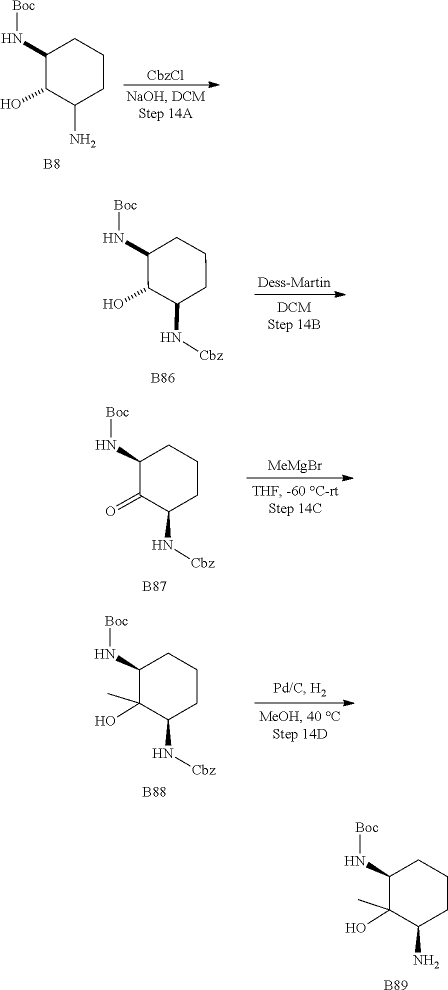

### Step 14A. Synthesis of benzyl tert-butyl (2-hydroxycyclohexane-1,3-diyl)dicarbamate (B86)

To a solution of tert-butyl (3-amino-2-hydroxycyclohexyl)carbamate (B8) (60 mg, 261 μmol) in DCM (3 mL) was added benzyl carbonochloridate (53.3 mg, 313 μmol, 44.4 μL), and then aq. NaOH solution (52.1 mg, 521 μmol, 1 M) was added. The reaction mixture was stirred at rt for 3 hrs and concentrated to dryness. EtOAc (10 mL) was added to the residue, and it was stirred for 1 hr. The organic layer was washed with brine (5 mL×3), dried over Na₂SO₄, filtered, and concentrated to dryness to afford B86 (90 mg), which was used without further purification. LC-MS: m/z 365.2 [M+H]⁺.

### Step 14B. Synthesis of benzyl tert-butyl (2-oxocyclohexane-1,3-diyl) dicarbamate (B87)

To a solution of tert-butyl (2-hydroxycyclohexane-1,3-diyl)dicarbamate (B86) (90 mg, 247 μmol) in DCM (2 mL) was added Dess-Martin reagent (115 mg, 272 μmol). The reaction mixture was stirred at rt for 3 hrs. The reaction mixture was quenched by the addition of sat. aq. NaHCO₃ solution (5 mL), and then H₂O (5 mL) was added. The aqueous portion was extracted with EtOAc (10 mL×3). The combined organic layers were washed with brine (10 mL×3), dried over Na₂SO₄, filtered, and concentrated to dryness. The residue was purified by column chromatography to give B87 (73 mg, 81.6% yield). LC-MS: m/z 363.2 [M+H]⁺.

### Step 14C. Synthesis of rel-benzyl tert-butyl ((1R,3S)-2-hydroxy-2-methylcyclohexane-1,3-diyl)dicarbamate (B88)

A mixture of benzyl tert-butyl (2-oxocyclohexane-1,3-diyl)dicarbamate (B87) (73 mg, 201 μmol) in THF (2 mL) was degassed and purged with N₂ for 3 times, and then MeMgBr (3 M, 235 μL) was added to the reaction mixture. The reaction mixture was then stirred at −60° C. for 2 hrs under N₂ atmosphere, and then stirred at rt for 1 hr under N₂ atmosphere. The reaction mixture was quenched by the addition of sat. aq. NH₄Cl solution (5 mL) at 10° C. Water (5 mL) was added, and the aqueous portion was extracted with EtOAc (10 mL×3). The combined organic layers were washed with brine (10 mL×3), dried over Na₂SO₄, filtered, and concentrated to dryness. The residue was purified by column chromatography to give B88 (40 mg, 52.5% yield). LC-MS: m/z 401.2 [M+Na]⁺.

### Step 4D. Synthesis of rel-tert-butyl ((1S,3R)-3-amino-2-hydroxy-2-methylcyclohexyl)-carbamate (B89)

A mixture of rel-benzyl tert-butyl ((1R,3S)-2-hydroxy-2-methylcyclohexane-1,3-diyl)dicarbamate (B88) (590 mg, 1.56 mmol) in MeOH (3 mL) was degassed and purged with N₂ for 3 times, and then Pd/C (400 mg, 10% purity) was added to the reaction mixture. The reaction mixture was degassed and purged with H₂ 3 times and stirred at 40° C. for 12 hrs under H₂ atmosphere (15 Psi). The reaction mixture was cooled to rt, filtered through a Celite pad, and the filter cake washed with MeOH (5 mL×3), The combined organic layers were concentrated to dryness to afford B89 (368 mg), which was used without further purification. The obtained product was one stereoisomer. The stereochemistry of the carbon with the methyl and hydroxy substituents has not been assigned.

### Intermediate Scheme 15: Preparation of rac-tert-butyl (1S,2R,3R)-3-aminocyclohexyl-2-d)carbamate and rac-tert-butyl ((1S,2S,3R)-3-aminocyclohexyl-2-d)carbamate

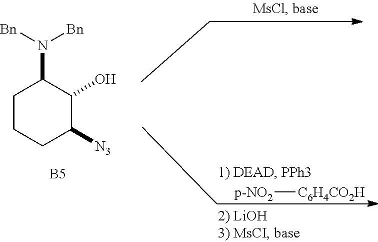

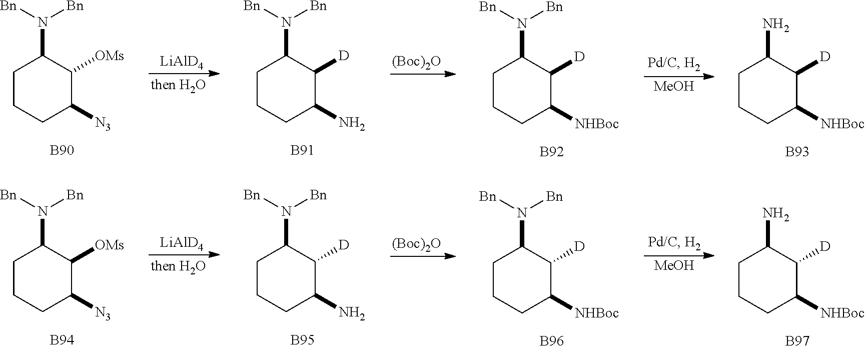

As indicated in Intermediate Scheme 15, rac-tert-butyl ((1S,2R,3R)-3-aminocyclohexyl-2-d)carbamate (B93) may be prepared by first reacting 2-azido-6-(dibenzylamino)cyclohexan-1-ol (B5, racemic) with methanesulfonyl chloride and a suitable base (such as TEA or pyridine) to afford rac-((1S,2S,6R)-2-azido-6-(dibenzylamino)cyclohexyl methanesulfonate (B90). The methanesulfonate group can be subsequently replaced with deuterium through a reduction reaction with lithium aluminum deuteride and water to afford rac-(1R,2S,3S)—N¹,N¹-dibenzylcyclohexane-2-d-1,3-diamine (B91). The free amine of B91 can be subsequently protected with a tert-butyl carbamate group to afford rac-tert-butyl ((1S,2S,3R)-3-(dibenzylamino)cyclohexyl-2-d)carbamate (B92). The dibenzyl groups of B92 can then be subsequently hydrogenated with Pd/C and H₂ gas in MeOH to afford rac-tert-butyl ((1S,2R,3R)-3-aminocyclohexyl-2-d)carbamate (B93). The enantiomers of B93 may be obtained via SFC separation.

Rac-tert-butyl ((1S,2S,3R)-3-aminocyclohexyl-2-d)carbamate (B97) may be prepared by inverting the stereochemistry of the hydroxyl group of B5 through reaction with DEAD, triphenylphosphine, and para-nitrobenzoic acid, followed by reaction with LiOH and methanesulfonyl chloride and a suitable base (such as TEA or pyridine) to afford rac-((1R,2S,6R)-2-azido-6-(dibenzylamino)cyclohexyl methanesulfonate (B94). The methanesulfonate group of B38 can be subsequently replaced with deuterium through a reduction reaction with lithium aluminum deuteride and water to afford rac-(1R,2R,3S)—N¹,N¹-dibenzylcyclohexane-2-d-1,3-diamine (B95). The free amine of B95 can be subsequently protected with a tert-butyl carbamate protecting group to afford rac-tert-butyl ((1S,2S,3R)-3-(dibenzylamino)cyclohexyl-2-d)carbamate (B96). The dibenzyl groups of B96 can then be subsequently hydrogenated with Pd/C and H₂ gas in MeOH to afford rac-tert-butyl ((1S,2S,3R)-3-aminocyclohexyl-2-d)carbamate (B97). The enantiomers of B97 may be obtained via SFC separation.

### Intermediate Scheme 16: Preparation of rac-(1R,3S,5s)-cyclohexane-5-d-1,3-diamine and rac-(1R,3S,5r)-cyclohexane-5-d-1,3-diamine

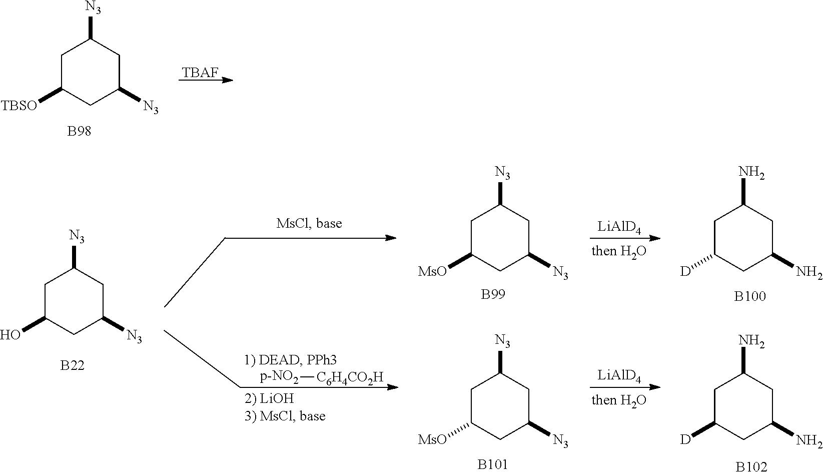

As indicated in Intermediate Scheme 16, rac-(1s,3R,5S)-3,5-diazidocyclohexan-1-ol (B22) may be prepared by removing the tert-butyldimethylsilyl ether protecting group of rac-tert-butyl(((1s,3R,5S)-3,5-diazidocyclohexyl)oxy)dimethylsilane (B98) with TBAF or another suitable desilylation reagent known in the art.

B22 may be subsequently reacted with methanesulfonyl chloride and a suitable base (such as TEA or pyridine) to afford rac-(1s,3R,5S)-3,5-diazidocyclohexyl methanesulfonate (B99). The methanesulfonate group can be subsequently replaced with deuterium through a reduction reaction with lithium aluminum deuteride and water to afford rac-(1R,3S,5s)-cyclohexane-5-d-1,3-diamine (B100). The enantiomers of B100 may be obtained via SFC separation.

Rac-(1R,3S,5r)-cyclohexane-5-d-1,3-diamine (B102) may be prepared by inverting the stereochemistry of the hydroxyl group of (1s,3R,5S)-3,5-diazidocyclohexan-1-ol (B22) through reaction with DEAD, triphenylphosphine, and para-nitrobenzoic acid, followed by reaction with LiOH and methanesulfonyl chloride and a suitable base (such as TEA or pyridine) to afford rac-(1r,3R,5S)-3,5-diazidocyclohexyl methanesulfonate (B101). The methanesulfonate group of B101 can be subsequently replaced with deuterium through a reduction reaction with lithium aluminum deuteride and water to afford rac-(1R,3S,5r)-cyclohexane-5-d-1,3-diamine (B102). The enantiomers of B102 may be obtained via SFC separation.

### Preparation of Intermediate (1S,3R)-3-(2-(2-(difluoromethoxy)phenyl)-6-(1H-1,2,4-triazol-3-yl)-1H-imidazo[4,5-c]pyridin-1-yl)cyclohexan-1-amine via General Scheme 2A (Methods A, C, and H)

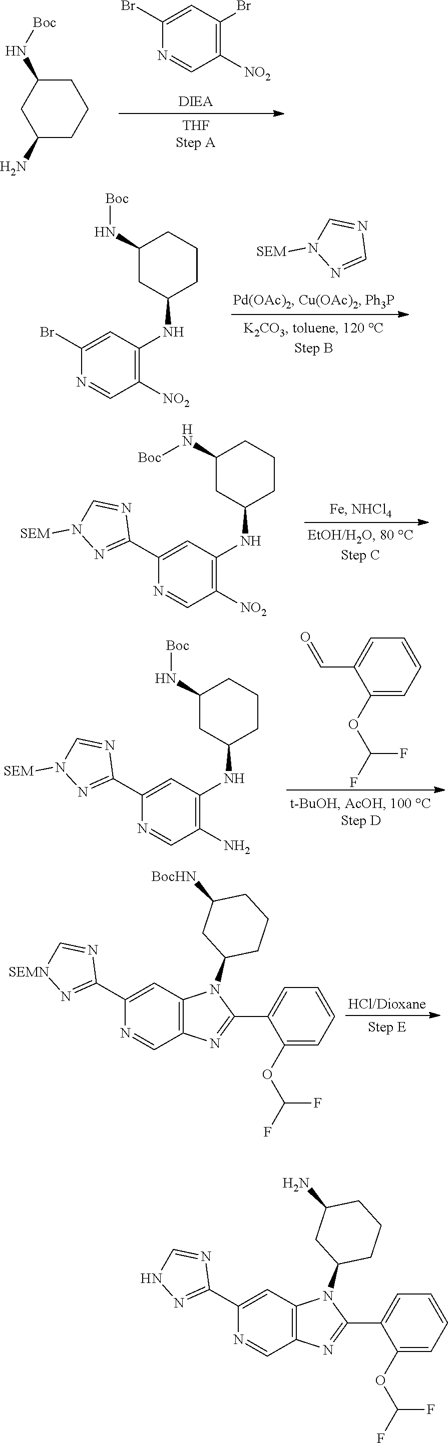

### Step A. Synthesis of tert-butyl ((1S,3R)-3-((2-bromo-5-nitropyridin-4-yl)amino)cyclohexyl)carbamate

To a solution of 2,4-dibromo-5-nitro-pyridine (3.95 g, 14.0 mmol) and tert-butyl ((1S,3R)-3-aminocyclohexyl)carbamate (3.0 g, 14.0 mmol) in THF (45 mL) was added DIPEA (5.43 g, 42.0 mmol). The reaction mixture was stirred at 70° C. for 15 hrs and then concentrated to dryness to afford tert-butyl ((1S,3R)-3-((2-bromo-5-nitropyridin-4-yl)amino)cyclohexyl)carbamate (9 g), which was used without further purification. LC-MS: m/z 417.1 [M+H]⁺.

### Step B. Synthesis of tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate

To a solution of tert-butyl ((1S,3R)-3-((2-bromo-5-nitropyridin-4-yl)amino)cyclohexyl)carbamate (11 g, 26 mmol), K₂CO₃ (7.3 g, 53 mmol) and 1-((2-(trimethylsilyl)ethoxy)methyl)-1H-1,2,4-triazole (6.3 g, 32 mmol) in toluene (100 mL) were added PPh₃ (3.5 g, 13 mmol), Cu(OAc)₂ (0.48 g, 2.6 mmol) and Pd(OAc)₂ (0.59 g, 2.6 mmol). The reaction mixture was degassed under vacuum and purged with N₂ several times, and then heated at 125° C. under N₂ atmosphere for 3 hrs. After completion, the reaction mixture was concentrated to dryness. The residue was purified by flash column chromatography on silica gel to afford tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate (II g, 78% yield). LC-MS: m/z 534 [M+H]⁺.

### Step C. Synthesis of tert-butyl ((1S,3R)-3-((5-amino-2-(1-((2-(trimethylsilyl)ethoxy)methyl)-1H-1,2,4-triazol-3-yl)pyridin-4-yl)amino)cyclohexyl)carbamate

To a solution of tert-butyl ((1S,3R)-3-((5-nitro-2-(1-((2-(trimethylsilyl)ethoxy)methyl)-1H-1,2,4-triazol-3-yl)pyridin-4-yl)amino)cyclohexyl)carbamate (7.9 g, 15 mmol) and NH₄Cl (7.9 g, 0.15 μmol) in EtOH/H₂O (90 mL/30 mL) was added Fe (8.3 g, 0.15 μmol). The reaction mixture was then heated at 80° C. under N₂ atmosphere for 1 hr. After completion, the reaction mixture was cooled to rt, filtered through a pad of Celite®, and the filter cake was washed with MeOH (20 mL). The filtrate was concentrated under reduced pressure, and water (80 mL) was added. The aqueous portion was extracted with EtOAc (100 mL×3). The combined organic layers were washed with brine (100 mL), dried over Na₂SO₄, filtered, and concentrated to dryness. The residue was purified by flash column chromatography on silica gel to afford tert-butyl ((1S,3R)-3-((5-amino-2-(1-((2-(trimethylsilyl)ethoxy)methyl)-1H-1,2,4-triazol-3-yl)pyridin-4-yl)amino)cyclohexyl)carbamate (5.8 g, 78% yield). LC-MS: m/z 504 [M+H]⁺.

### Step D. Synthesis of tert-butyl ((1S,3R)-3-(2-(2-(difluoromethoxy)phenyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl ((1S,3R)-3-((5-amino-2-(1-((2-(trimethylsilyl)ethoxy)methyl)-1H-1,2,4-triazol-3-yl)pyridin-4-yl)amino)cyclohexyl)carbamate (300 mg, 596 μmol) and 2-(difluoromethoxy)benzaldehyde (205 mg, 1.19 mmol) in t-BuOH (10 mL) was added AcOH (2 mL), and the reaction mixture was stirred at 100° C. for 16 hrs. After completion, water (10 mL) was added to the reaction mixture at rt, and the aqueous portion extracted with EtOAc (20 mL×3). The combined organic layers were washed with brine (15 mL), dried over Na₂SO₄, filtered, and concentrated to dryness. The residue was purified by flash column chromatography on silica gel to afford tert-butyl ((1S,3R)-3-(2-(2-(difluoromethoxy)phenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (285 mg, 73% yield). LC-MS: m/z 656 [M+H]⁺.

### Step E. Synthesis of (1S,3R)-3-(2-(2-(difluoromethoxy)phenyl)-6-(1H-1,2,4-triazol-3-yl)-1H-imidazo[4,5-c]pyridin-1-yl)cyclohexan-1-amine

A solution of tert-butyl ((1S,3R)-3-(2-(2-(difluoromethoxy)phenyl)-6-(1-((2-(trimethylsilyl)ethoxy)methyl)-1H-1,2,4-triazol-3-yl)-1H-imidazo[4,5-c]pyridin-1-yl)cyclohexyl)carbamate (285 mg, 435 μmol) in HCl/dioxane (3 mL, 4M), was stirred at rt for 3 hrs. After completion, the reaction mixture was concentrated to dryness to afford (1S,3R)-3-(2-(2-(difluoromethoxy)phenyl)-6-(1H-1,2,4-triazol-3-yl)-1H-imidazo[4,5-c]pyridin-1-yl)cyclohexan-1-amine (310 mg), which was used without further purification. LC-MS: m/z 426 [M+H]⁺.

### Preparation of Intermediate (1S,3R)-3-(2-(2-fluorophenyl)-6-(2H-1,2,3-triazol-2-yl)-1H-imidazo[4,5-c]pyridin-1-yl)cyclohexan-1-amine via General Scheme 2A (Methods A, F, and H)

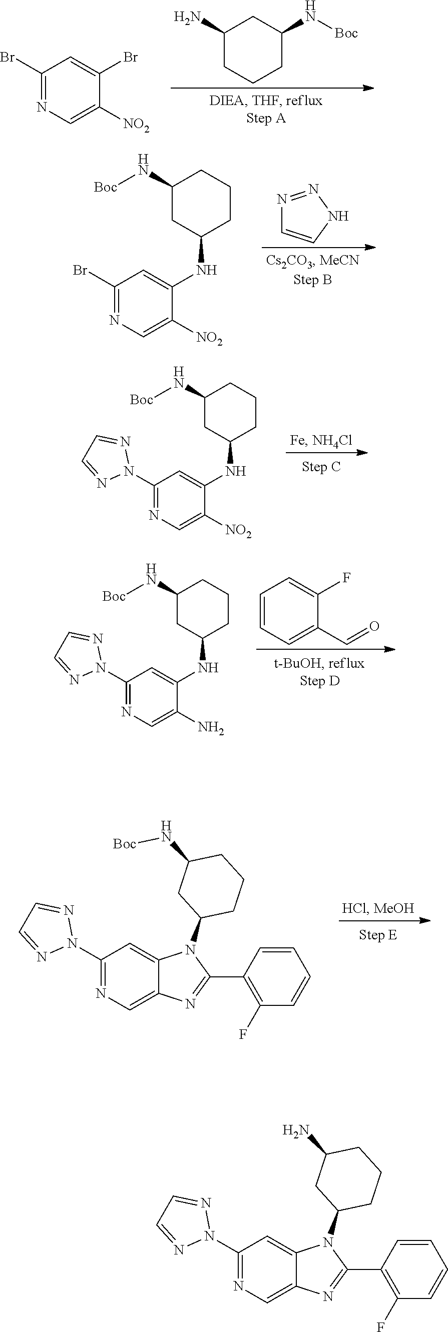

### Step A. Synthesis of tert-butyl ((1S,3R)-3-((2-bromo-5-nitropyridin-4-yl)amino)cyclohexyl)carbamate

To a solution of 2,4-dibromo-5-nitro-pyridine (3.95 g, 14.0 mmol) and tert-butyl ((1S,3R)-3-aminocyclohexyl)carbamate (3.0 g, 14.0 mmol) in THF (45 mL) was added DIEA (5.43 g, 42.0 mmol). The reaction mixture was stirred at 70° C. for 15 hrs, and then concentrated to dryness to give tert-butyl ((1S,3R)-3-((2-bromo-5-nitropyridin-4-yl)amino)cyclohexyl)carbamate (9 g), which was used without further purification. LC-MS: m/z 417.1 [M+H]⁺.

### Step B. Synthesis of tert-butyl ((1S,3R)-3-((5-nitro-2-(2H-1,2,3-triazol-2-yl)pyridin-4-yl)amino)cyclohexyl)carbamate

To a solution of tert-butyl ((1S,3R)-3-((2-bromo-5-nitropyridin-4-yl)amino)cyclohexyl)carbamate (1.1 g, 2.65 mmol) and 1H-triazole (220 mg, 3.18 mmol) in MeCN (14 mL) was added Cs₂CO₃ (1.73 g, 5.30 mmol). The reaction mixture was stirred at 80° C. for 15 hrs, and then allowed to cool. The resulting slurry was filtered, and the filtrate was concentrated to give a residue. The residue was purified by flash silica gel chromatography to give (1) tert-butyl ((1S,3R)-3-((5-nitro-2-(1H-1,2,3-triazol-1-yl)pyridin-4-yl)amino)cyclohexyl)carbamate (710 mg, 33.2% yield) (LC-MS: m/z 404.2 [M+H]⁺) and (2) tert-butyl ((1S,3R)-3-((5-nitro-2-(2H-1,2,3-triazol-2-yl)pyridin-4-yl)amino)cyclohexyl)carbamate (770 mg, 36.0% yield). ¹H NMR (400 MHz, CDCl₃) δ 9.25 (s, 1H), 8.31 (d, 1H), 7.95 (s, 2H), 7.53 (s, 1H), 4.49 (br s, 1H), 3.76-3.68 (m, 2H), 2.51-2.47 (m, 1H), 2.20-2.16 (m, 1H), 2.06-2.02 (m, 1H), 1.94-1.92 (m, 1H), 1.54-1.51 (m, 1H), 1.45 (s, 9H), 1.29-1.16 (m, 3H); LC-MS: m/z 404.2 [M+H]⁺.

### Step C. Synthesis of tert-butyl ((1S,3R)-3-((5-amino-2-(2H-1,2,3-triazol-2-yl)pyridin-4-yl)amino)cyclohexyl)carbamate

To a solution of tert-butyl ((1S,3R)-3-((5-nitro-2-(2H-1,2,3-triazol-2-yl)pyridin-4-yl)amino)cyclohexyl)carbamate (770 mg, 1.91 mmol) in THF (8 mL) were added Fe (533 mg, 9.54 mmol) and NH₄Cl (123 mg, 2.29 mmol) in water (4 mL). The reaction mixture was stirred at 70° C. for 15 hrs, and then allowed to cool. The reaction mixture was filtered, and the filtrate concentrated to dryness to give tert-butyl ((1S,3R)-3-((5-amino-2-(2H-1,2,3-triazol-2-yl)pyridin-4-yl)amino)cyclohexyl)carbamate (750 mg), which was used without further purification. LC-MS: m/z 374.2 [M+H]⁺.

### Step D. Synthesis of tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate

To a solution of tert-butyl ((1S,3R)-3-((5-amino-2-(2H-1,2,3-triazol-2-yl)pyridin-4-yl)amino)cyclohexyl)carbamate (100 mg, 268 μmol) in t-BuOH (3 mL) was added 2-fluorobenzaldehyde (39.9 mg, 321 μmol) at rt. The reaction mixture was stirred at 90° C. for 16 hrs, and then allowed to cool. The reaction mixture was concentrated to dryness to give a residue, which was purified by flash silica gel chromatography to give tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate (60 mg, 42.2% yield). LC-MS: m/z 478.2 [M+H]⁺.

### Step E. Synthesis of (1S,3R)-3-(2-(2-fluorophenyl)-6-(2H-1,2,3-triazol-2-yl)-1H-imidazo[4,5-c]pyridin-1-yl)cyclohexan-1-amine

To a solution of tert-butyl ((1S,3R)-3-(2-(2-fluorophenyl)-6-(2H-1,2,3-triazol-2-yl)-1H-imidazo[4,5-c]pyridin-1-yl)cyclohexyl)carbamate (60 mg, 113 μmol) in MeOH (1 mL) was added HCl/dioxane (4M, 2 mL). The reaction mixture was stirred at rt for 1 hr, and then concentrated to dryness to give (1S,3R)-3-(2-(2-fluorophenyl)-6-(2H-1,2,3-triazol-2-yl)-1H-imidazo[4,5-c]pyridin-1-yl)cyclohexan-1-amine (50 mg, 2HCl salt), which was used without further purification. LC-MS: m/z 378.2 [M+H]⁺.

### Preparation of Intermediate 2-amino-6-(2-(2-fluorophenyl)-6-(2H-1,2,3-triazol-2-yl)-1H-imidazo[4,5-c]pyridin-1-yl)cyclohexan-1-ol via General Scheme 2A (Methods A, F, and H) (relative stereochemistry is indicated in scheme)

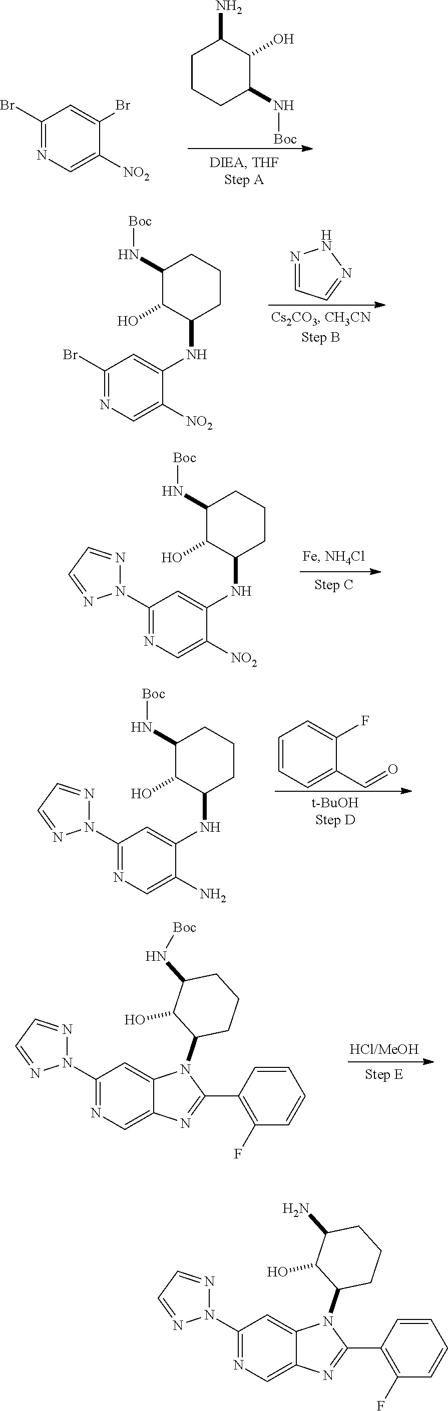

### Step A. Synthesis of tert-butyl (3-((2-bromo-5-nitropyridin-4-yl)amino)-2-hydroxycyclohexyl)carbamate

To a solution of tert-butyl (3-amino-2-hydroxycyclohexyl)carbamate (500 mg, 2.17 mmol) (product of Step 1G in Intermediate Scheme 1 above) in THF (20 mL) were added DIEA (842 mg, 6.51 mmol) and 2,4-dibromo-5-nitro-pyridine (612 mg, 2.17 mmol). The reaction mixture was stirred at 70° C. for 15 hrs. The reaction mixture was concentrated to dryness. The residue was purified by silica gel column chromatography to give tert-butyl (3-((2-bromo-5-nitropyridin-4-yl)amino)-2-hydroxycyclohexyl)carbamate (157 mg, 16.8% yield). ¹H NMR (400 MHz, CDCl₃) δ 8.93 (s, 1H), 8.23 (br d, 1H), 7.16 (s, 1H), 4.64 (br s, 1H), 4.23 (br s, 1H), 3.60-3.48 (m, 2H), 3.38-3.33 (m, 1H), 2.09-2.00 (m, 3H), 1.91-1.84 (m, 1H), 1.45 (s, 9H), 1.39-1.30 (m, 2H); LC-MS: m/z 431.2 [M+H]⁺.

### Step B. Synthesis of tert-butyl (2-hydroxy-3-((5-nitro-2-(2H-1,2,3-triazol-2-yl)pyridin-4-yl)amino)cyclohexyl)carbamate

To a solution of tert-butyl (3-((2-bromo-5-nitropyridin-4-yl)amino)-2-hydroxycyclohexyl)carbamate (250 mg, 580 μmol) in MeCN (3 mL) were added Cs₂CO₃ (472 mg, 1.45 mmol) and 2H-triazole (48.0 mg, 696 μmol). The reaction mixture was stirred at 70° C. for 12 hrs, and then allowed to cool. The reaction mixture was quenched by the addition of H₂O (5 mL) at rt, diluted with H₂O (10 mL), and the aqueous portion extracted with EtOAc (10 mL×3). The combined organic layers were washed with brine (10 mL×3), dried over Na₂SO₄, filtered, and concentrated to dryness. The residue was purified by silica gel column chromatography to give tert-butyl (2-hydroxy-3-((5-nitro-2-(2H-1,2,3-triazol-2-yl)pyridin-4-yl)amino)cyclohexyl)carbamate (50 mg, 20.6% yield). LC-MS: m/z 420.2 [M+H]⁺.

### Step C. Synthesis of tert-butyl (3-((5-amino-2-(2H-1,2,3-triazol-2-yl)pyridin-4-yl)amino)-2-hydroxycyclohexyl)carbamate

To a solution of tert-butyl (2-hydroxy-3-((5-nitro-2-(2H-1,2,3-triazol-2-yl)pyridin-4-yl)amino)cyclohexyl)carbamate (35 mg, 83 μmol) in EtOH (0.5 mL) and H₂O (0.2 mL) were added Fe (23.3 mg, 417 μmol) and NH₄Cl (6.70 mg, 125 μmol). The reaction mixture was stirred at 80° C. for 1 hr. The reaction mixture was concentrated under reduced pressure to remove volatile solvents. The residue was diluted with THF (5 mL) and then filtered. The filter cake was washed with THF (20 mL×3). The combined organic layers were filtered again, and the filtrate concentrated to dryness to give tert-butyl (3-((5-amino-2-(2H-1,2,3-triazol-2-yl)pyridin-4-yl)amino)-2-hydroxycyclohexyl)carbamate (32 mg), which was used without further purification. LC-MS: m/z 390.3 [M+H]⁺.

### Step D. Synthesis of tert-butyl (3-(2-(2-fluorophenyl)-6-(2H-1,2,3-triazol-2-yl)-1H-imidazo[4,5-c]pyridin-1-yl)-2-hydroxycyclohexyl)carbamate

To a solution of tert-butyl (3-((5-amino-2-(2H-1,2,3-triazol-2-yl)pyridin-4-yl)amino)-2-hydroxycyclohexyl)carbamate (32 mg, 82 μmol) in t-BuOH (0.5 mL) was added 2-fluorobenzaldehyde (12.2 mg, 98.6 μmol). The reaction mixture was stirred at 85° C. for 20 hrs. The reaction mixture was concentrated to dryness. The residue was purified by silica gel column chromatography to give tert-butyl (3-(2-(2-fluorophenyl)-6-(2H-1,2,3-triazol-2-yl)-1H-imidazo[4,5-c]pyridin-1-yl)-2-hydroxycyclohexyl)carbamate (28 mg, 60.8% yield). LC-MS: m/z 494.2 [M+H]⁺.

### Step E. Synthesis of 2-amino-6-(2-(2-fluorophenyl)-6-(2H-1,2,3-triazol-2-yl)-1H-imidazo[4,5-c]pyridin-1-yl)cyclohexan-1-ol

To a solution of tert-butyl (3-(2-(2-fluorophenyl)-6-(2H-1,2,3-triazol-2-yl)-1H-imidazo[4,5-c]pyridin-1-yl)-2-hydroxycyclohexyl)carbamate (28.0 mg, 56.7 μmol) in MeOH (0.5 mL) was added HCl/MeOH (4 M, 1 mL). The reaction mixture was stirred at rt for 1 hr and then concentrated to dryness to give 2-amino-6-(2-(2-fluorophenyl)-6-(2H-1,2,3-triazol-2-yl)-1H-imidazo[4,5-c]pyridin-1-yl)cyclohexan-1-ol (23 mg), which was used without further purification. LC-MS: m/z 394.2 [M+H]⁺.

### Preparation of Intermediate (1R,2S,6R)-2-amino-6-(2-(2-fluorophenyl)-6-(1H-1,2,4-triazol-3-yl)-1H-imidazo[4,5-c]pyridin-1-yl)cyclohexan-1-ol via General Scheme 2A (Methods A, C, and H)

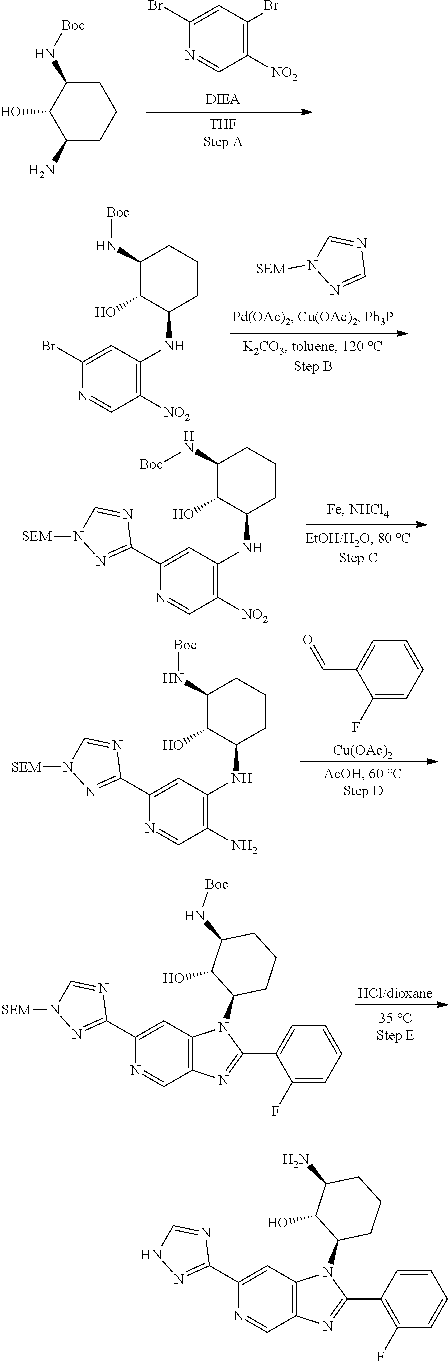

### Step A. Synthesis of tert-butyl ((1S,2S,3R)-3-((2-bromo-5-nitropyridin-4-yl)amino)-2-hydroxycyclohexyl)carbamate

To a solution of tert-butyl ((1S,2S,3R)-3-amino-2-hydroxycyclohexyl)carbamate (3.2 g, 14 mmol) in THF (50 mL) were added DIPEA (5.4 g, 42 mmol) and 2,4-dibromo-5-nitropyridine (4.3 g, 15 mmol) at 0° C. The reaction mixture was stirred at rt for 15 hrs. After completion, water (20 mL) was added to the reaction mixture, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1S,2S,3R)-3-((2-bromo-5-nitropyridin-4-yl)amino)-2-hydroxycyclohexyl)carbamate (5.0 g, 83% yield). LC-MS: m/z 431 [M+H]⁺.

### Step B. Synthesis of tert-butyl ((1S,2S,3R)-2-hydroxy-3-((5-nitro-2-(1-((2-(trimethylsilyl)ethoxy)methyl)-1H-1,2,4-triazol-3-yl)pyridin-4-yl)amino)cyclohexyl)carbamate

To a mixture of tert-butyl ((1S,2S,3R)-3-((2-bromo-5-nitropyridin-4-yl)amino)-2-hydroxycyclohexyl)carbamate (5.7 g, 13 mmol), 1-((2-(trimethylsilyl)ethoxy)methyl)-1H-1,2,4-triazole (2.6 g, 13 mmol), triphenylphosphine (1.7 g, 6.6 mmol), copper(II) acetate (0.48 g, 2.6 mmol) and K₂CO₃ (3.7 g, 26 mmol) in toluene (60 mL) was added Pd(OAc)₂ (0.15 g, 0.66 mmol). The reaction mixture was degassed under vacuum and purged with N₂ several times, and then stirred at 120° C. under N₂ atmosphere for 16 hrs. After completion, the reaction mixture was concentrated to dryness. The residue was purified by flash column chromatography on silica gel to afford tert-butyl ((1S,2S,3R)-2-hydroxy-3-((5-nitro-2-(1-((2-(trimethylsilyl)ethoxy)methyl)-1H-1,2,4-triazol-3-yl)pyridin-4-yl)amino)cyclohexyl)carbamate (5 g, 70% yield). LC-MS: m/z 550 [M+H]⁺.

### Step C. Synthesis of tert-butyl ((1S,2S,3R)-3-((5-amino-2-(1-((2-(trimethylsilyl)ethoxy)methyl)-1H-1,2,4-triazol-3-yl)pyridin-4-yl)amino)-2-hydroxycyclohexyl)carbamate

To a mixture of tert-butyl ((1S,2S,3R)-2-hydroxy-3-((5-nitro-2-(1-((2-(trimethylsilyl)ethoxy)methyl)-1H-1,2,4-triazol-3-yl)pyridin-4-yl)amino)cyclohexyl)carbamate (5 g, 9 mmol) and NH₄Cl (2 g, 0.05 mol) in EtOH/H₂O (100 mL/50 mL) was added Fe (3 g, 0.05 mol). The reaction mixture was stirred at 80° C. under N₂ atmosphere for 1 hr. After completion, the reaction mixture was cooled to rt and filtered through a pad of Celite®. The filter cake was washed with MeOH (20 mL), and the filtrate concentrated to dryness. The residue was purified by flash column chromatography on silica gel to afford tert-butyl ((1S,2S,3R)-3-((5-amino-2-(1-((2-(trimethylsilyl)ethoxy)methyl)-1H-1,2,4-triazol-3-yl)pyridin-4-yl)amino)-2-hydroxycyclohexyl)carbamate (4.5 g, 97% yield). LC-MS: m/z 520 [M+H]⁺.

### Step D. Synthesis of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate

To a mixture of tert-butyl ((1R,2R,3S)-3-((5-amino-2-(1-((2-(trimethylsilyl)ethoxy)methyl)-1H-1,2,4-triazol-3-yl)pyridin-4-yl)amino)-2-hydroxycyclohexyl)carbamate (8 g, 15.4 mmol) in AcOH (60 mL) was added 2-fluorobenzaldehyde (2.87 g, 23.1 mmol) and then the reaction mixture was stirred at 60° C. for 3 hrs. Cu(OAc)₂ (2.8 g, 15.4 mmol) was added, and the reaction mixture was stirred at 60° C. for 24 hrs. After completion, the reaction mixture was cooled to rt and filtered. Water (40 mL) was added to the filtrate, and the aqueous portion extracted with EtOAc (60 mL×3). The combined organic layers were washed with brine (15 mL), dried over Na₂SO₄, filtered, and concentrated to dryness. The residue was purified by flash column chromatography on silica gel to afford tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (6.5 g, 58% yield). LC-MS: m/z 624 [M+H]⁺.

### Step E. Synthesis of (1R,2S,6R)-2-amino-6-(2-(2-fluorophenyl)-6-(1H-1,2,4-triazol-3-yl)-1H-imidazo[4,5-c]pyridin-1-yl)cyclohexan-1-ol

A solution of tert-butyl ((1S,3R)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-2-oxocyclohexyl)carbamate (150 mg, 240 μmol) in HCl/dioxane (2 mL, 4M) was stirred at 35° C. for 1 hr. After completion, the reaction mixture was concentrated to dryness to afford (1R,2S,6R)-2-amino-6-(2-(2-fluorophenyl)-6-(1H-1,2,4-triazol-3-yl)-1H-imidazo[4,5-c]pyridin-1-yl)cyclohexan-1-ol (100 mg), which was used without further purification. LC-MS: m/z 394 [M+H]⁺.

### Preparation of Intermediate (1R,3S,5S)-3-(2-(2-fluorophenyl)-6-(1H-1,2,4-triazol-3-yl)-1H-imidazo[4,5-c]pyridin-1-yl)-5-methoxycyclohexan-1-amine via General Scheme 2A (Methods A, C, and H)

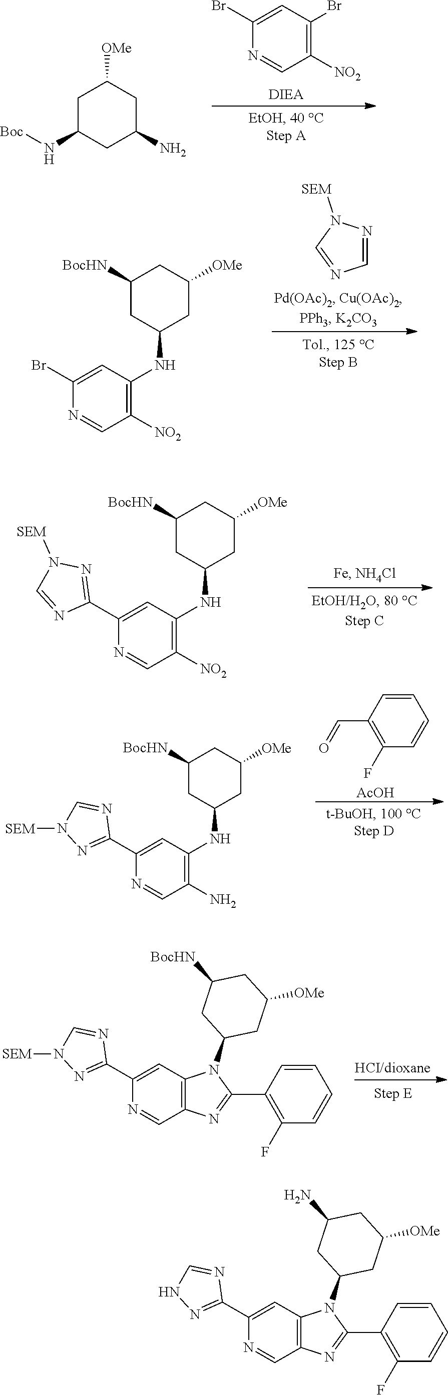

### Step A. Synthesis of tert-butyl ((1R,3S,5R)-3-((2-bromo-5-nitropyridin-4-yl)amino)-5-methoxycyclohexyl)carbamate

To a solution of tert-butyl ((1R,3S,5R)-3-amino-5-methoxycyclohexyl)carbamate (1.9 g, 7.8 mmol) and 2,4-dibromo-5-nitropyridine (2.2 g, 7.8 mmol) in EtOH (20 mL) was added DIEA (2 g, 15.6 mmol), and the reaction mixture was stirred at 40° C. for 16 hrs. After completion, water (50 mL) was added to the reaction mixture at rt, and the aqueous portion extracted with EtOAc (50 mL×2). The combined organic layers were washed with brine (30 mL), dried over Na₂SO₄, filtered and concentrated to dryness. The residue was purified by flash column chromatography on silica gel to give tert-butyl ((1R,3S,5R)-3-((2-bromo-5-nitropyridin-4-yl)amino)-5-methoxycyclohexyl)carbamate (4.7 g, 80% yield). LC-MS: m/z 445 [M+H]⁺.

### Step B. Synthesis of tert-butyl ((1R,3R,5S)-3-methoxy-5-((5-nitro-2-(1-((2-(trimethylsilyl)ethoxy)methyl)-1H-1,2,4-triazol-3-yl)pyridin-4-yl)amino)cyclohexyl)carbamate

To a solution of tert-butyl ((1R,3S,5R)-3-((2-bromo-5-nitropyridin-4-yl)amino)-5-methoxycyclohexyl)carbamate (773 mg, 1.74 mmol), K₂CO₃ (480 mg, 3.47 mmol) and 1-((2-(trimethylsilyl)ethoxy)methyl)-1H-1,2,4-triazole (415 mg, 2.08 mmol) in toluene (20 mL) were added PPh₃ (228 mg, 868 μmol), Cu(OAc)₂ (32 mg, 0.17 mmol) and Pd(OAc)₂ (20 mg, 0.87 mmol). The reaction mixture was degassed under vacuum and purged with N₂ several times, then heated at 125° C. under N₂ atmosphere for 3 hrs. After completion, the reaction mixture was concentrated to dryness. The residue was purified by flash column chromatography on silica gel to afford tert-butyl ((1R,3R,5S)-3-methoxy-5-((5-nitro-2-(1-((2-(trimethylsilyl)ethoxy)methyl)-1H-1,2,4-triazol-3-yl)pyridin-4-yl)amino)cyclohexyl)carbamate (682 mg, 70% yield). LC-MS: m/z 564 [M+H]⁺.

### Step C. Synthesis of tert-butyl ((1R,3S,5R)-3-((5-amino-2-(1-((2-(trimethylsilyl)ethoxy)methyl)-1H-1,2,4-triazol-3-yl)pyridin-4-yl)amino)-5-methoxycyclohexyl)carbamate

To a solution of tert-butyl ((1R,3R,5S)-3-methoxy-5-((5-nitro-2-(1-((2-(trimethylsilyl)ethoxy)methyl)-1H-1,2,4-triazol-3-yl)pyridin-4-yl)amino)cyclohexyl)carbamate (682 mg, 1.21 mmol) and NH₄Cl (324 mg, 6.05 mmol) in EtOH/H₂O (10 mL/5 mL) was added Fe (338 mg, 6.05 mmol). The reaction mixture was then heated at 80° C. under N₂ atmosphere for 1 hr. After completion, the reaction mixture was cooled to rt and filtered through a pad of Celite®. The filter cake was washed with MeOH (20 mL), and the filtrate was concentrated under reduced pressure. Water (15 mL) was added to the residue, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1R,3S,5R)-3-((5-amino-2-(1-((2-(trimethylsilyl)ethoxy)methyl)-1H-1,2,4-triazol-3-yl)pyridin-4-yl)amino)-5-methoxycyclohexyl)carbamate (557 mg, 86% yield). LC-MS: m/z 534 [M+H]⁺.

### Step D. Synthesis of tert-butyl ((1R,3S,5S)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-5-methoxycyclohexyl)carbamate

To a solution of tert-butyl ((1R,3S,5R)-3-((5-amino-2-(1-((2-(trimethylsilyl)ethoxy)methyl)-1H-1,2,4-triazol-3-yl)pyridin-4-yl)amino)-5-methoxycyclohexyl)carbamate (1.75 g, 3.28 mmol) in t-BuOH (30 mL) were added 2-fluorobenzaldehyde (610 mg, 4.92 mmol) and AcOH (0.6 mL), the reaction mixture was stirred at 100° C. for 48 hrs. After completion, the reaction mixture was concentrated to dryness. The residue was purified by flash column chromatography on silica gel to afford tert-butyl ((1R,3S,5S)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-5-methoxycyclohexyl)carbamate (2 g, 99% yield). LC-MS: m/z 638 [M+H]⁺.

### Step E. Synthesis of (1R,3S,5S)-3-(2-(2-fluorophenyl)-6-(1H-1,2,4-triazol-3-yl)-1H-imidazo[4,5-c]pyridin-1-yl)-5-methoxycyclohexan-1-amine

To a solution of tert-butyl ((1R,3S,5S)-3-(2-(2-fluorophenyl)-6-(1-((2-(trimethylsilyl)ethoxy)methyl)-1H-1,2,4-triazol-3-yl)-1H-imidazo[4,5-c]pyridin-1-yl)-5-methoxycyclohexyl)carbamate (900 mg, 1.41 mmol) in MeOH (5 mL) was added HCl/dioxane (5 mL, 4M), the mixture was stirred at rt for 2 hrs. After completion, the reaction mixture was concentrated to dryness to afford (1R,3S,5S)-3-(2-(2-fluorophenyl)-6-(1H-1,2,4-triazol-3-yl)-1H-imidazo[4,5-c]pyridin-1-yl)-5-methoxycyclohexan-1-amine (800 mg), which was used without further purification. LC-MS: m/z 408 [M+H]⁺.

### Preparation of Intermediate (1R,2S,6R)-2-amino-6-(2-(2-fluorophenyl)-6-(4-methyloxazol-2-yl)-1H-imidazo[4,5-c]pyridin-1-yl)cyclohexan-1-ol via General Scheme 2A (Methods D and H)

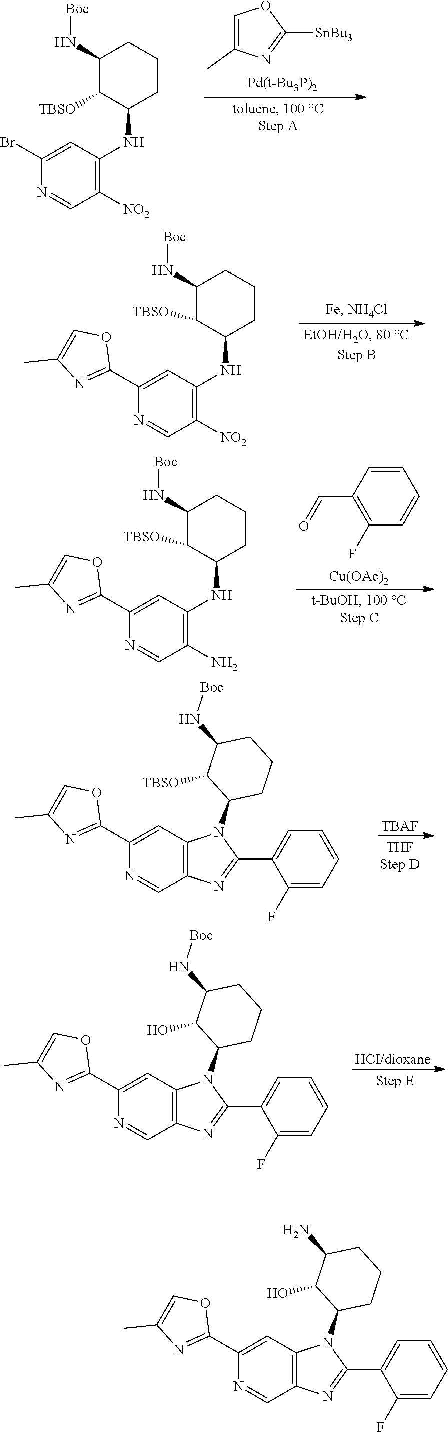

### Step A. Synthesis of tert-butyl ((1S,2S,3R)-2-((tert-butyldimethylsilyl)oxy)-3-((2-(4-methyloxazol-2-yl)-5-nitropyridin-4-yl)amino)cyclohexyl)carbamate

To a solution of tert-butyl ((1S,2S,3R)-3-((2-bromo-5-nitropyridin-4-yl)amino)-2-((tert-butyldimethylsilyl)oxy)cyclohexyl)carbamate (500 mg, 917 μmol) and 4-methyl-2-(tributylstannyl)oxazole (1.02 g, 2.75 mmol) in toluene (5 mL) was added Pd(t-Bu₃P)₂ (94 mg, 183 μmol). The reaction mixture was degassed under vacuum and purged with N₂ several times, then heated at 100° C. in a sealed tube under N₂ atmosphere for 16 hrs. After completion, the reaction mixture was concentrated to dryness. The residue was purified by flash column chromatography on silica gel to afford tert-butyl ((1S,2S,3R)-2-((tert-butyldimethylsilyl)oxy)-3-((2-(4-methyloxazol-2-yl)-5-nitropyridin-4-yl)amino)cyclohexyl)carbamate (300 mg, 60% yield). LC-MS: m/z 548 [M+H]⁺.

### Step B. Synthesis of tert-butyl ((1S,2S,3R)-3-((5-amino-2-(4-methyloxazol-2-yl)pyridin-4-yl)amino)-2-((tert-butyldimethylsilyl)oxy)cyclohexyl)carbamate

To a solution of tert-butyl ((1S,2S,3R)-2-((tert-butyldimethylsilyl)oxy)-3-((2-(4-methyloxazol-2-yl)-5-nitropyridin-4-yl)amino)cyclohexyl)carbamate (250 mg, 456 mol) and NH₄Cl (244 mg, 4.56 mmol) in EtOH/H₂O (10 mL/10 mL) was added Fe (255 mg, 4.56 mmol). The reaction mixture was then heated at 80° C. under N₂ atmosphere for 2 hrs. After completion, the reaction mixture was cooled to rt and filtered through a pad of Celite®. The filter cake was washed with MeOH (20 mL), and the filtrate was concentrated under reduced pressure. Water (15 mL) was added to the residue, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1S,2S,3R)-3-((5-amino-2-(4-methyloxazol-2-yl)pyridin-4-yl)amino)-2-((tert-butyldimethylsilyl)oxy)cyclohexyl)carbamate (160 mg, 68% yield). LC-MS: m/z 518 [M+H]⁺.

### Step C. Synthesis of tert-butyl ((1S,2R,3R)-2-((tert-butyldimethylsilyl)oxy)-3-(2-(2-fluorophenyl)-6-(4-methyloxazol-2-yl)-1H-imidazo[4,5-c]pyridin-1-yl)cyclohexyl)carbamate

To a solution of tert-butyl ((1S,2S,3R)-3-((5-amino-2-(4-methyloxazol-2-yl)pyridin-4-yl)amino)-2-((tert-butyldimethylsilyl)oxy)cyclohexyl)carbamate (210 mg, 0.41 mmol) in t-BuOH (5 mL) were added 2-fluorobenzaldehyde (76 mg, 0.62 mmol) and Cu(OAc)₂ (75 mg, 0.41 mmol). Then the reaction mixture was stirred at 100° C. for 16 hrs. After completion, the reaction mixture was cooled to rt. Water (10 mL) was added, and the aqueous portion extracted with EtOAc (30 mL×3). The combined organic layers were washed with brine (10 mL), dried over Na₂SO₄, filtered, and concentrated to dryness. The residue was purified by flash column chromatography on silica gel to give tert-butyl ((1S,2R,3R)-2-((tert-butyldimethylsilyl)oxy)-3-(2-(2-fluorophenyl)-6-(4-methyloxazol-2-yl)-1H-imidazo[4,5-c]pyridin-1-yl)cyclohexyl)carbamate (100 mg, 39% yield). LC-MS: m/z 622 [M+H]⁺.

### Step D. Synthesis of tert-butyl ((1S,2R,3R)-3-(2-(2-fluorophenyl)-6-(4-methyloxazol-2-yl)-1H-imidazo[4,5-c]pyridin-1-yl)-2-hydroxycyclohexyl)carbamate

To a solution of tert-butyl ((1S,2R,3R)-2-((tert-butyldimethylsilyl)oxy)-3-(2-(2-fluorophenyl)-6-(4-methyloxazol-2-yl)-1H-imidazo[4,5-c]pyridin-1-yl)cyclohexyl)carbamate (65 mg, 0.10 mmol) in THF (4 mL) was added TBAF (27 mg, 0.10 mmol). The reaction mixture was stirred at rt for 4 hrs. After completion, water (10 mL) was added, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to give tert-butyl ((1S,2R,3R)-3-(2-(2-fluorophenyl)-6-(4-methyloxazol-2-yl)-1H-imidazo[4,5-c]pyridin-1-yl)-2-hydroxycyclohexyl)carbamate (45 mg, 85% yield). LC-MS: m/z 508 [M+H]⁺.

### Step E. Synthesis of (1R,2S,6R)-2-amino-6-(2-(2-fluorophenyl)-6-(4-methyloxazol-2-yl)-1H-imidazo[4,5-c]pyridin-1-yl)cyclohexan-1-ol

A solution of tert-butyl ((1S,2R,3R)-3-(2-(2-fluorophenyl)-6-(4-methyloxazol-2-yl)-1H-imidazo[4,5-c]pyridin-1-yl)-2-hydroxycyclohexyl)carbamate (45 mg, 89 μmol) in HCl/dioxane(4 M) (4 mL) was stirred at rt for 4 hrs. After completion, the reaction mixture was concentrated to dryness to afford (1R,2S,6R)-2-amino-6-(2-(2-fluorophenyl)-6-(4-methyloxazol-2-yl)-1H-imidazo[4,5-c]pyridin-1-yl)cyclohexan-1-ol, which was used without further purification. LC-MS: m/z 408 [M+H]⁺.

### Preparation of intermediate 2-(2-(1-(5-amino-3,3-difluorocyclohexyl)-6-(1H-1,2,4-triazol-3-yl)-1H-benzo[d]imidazol-2-yl)phenoxy)ethan-1-ol via General Scheme 2A (Methods C and H) (relative stereochemistry in shown in the scheme below)

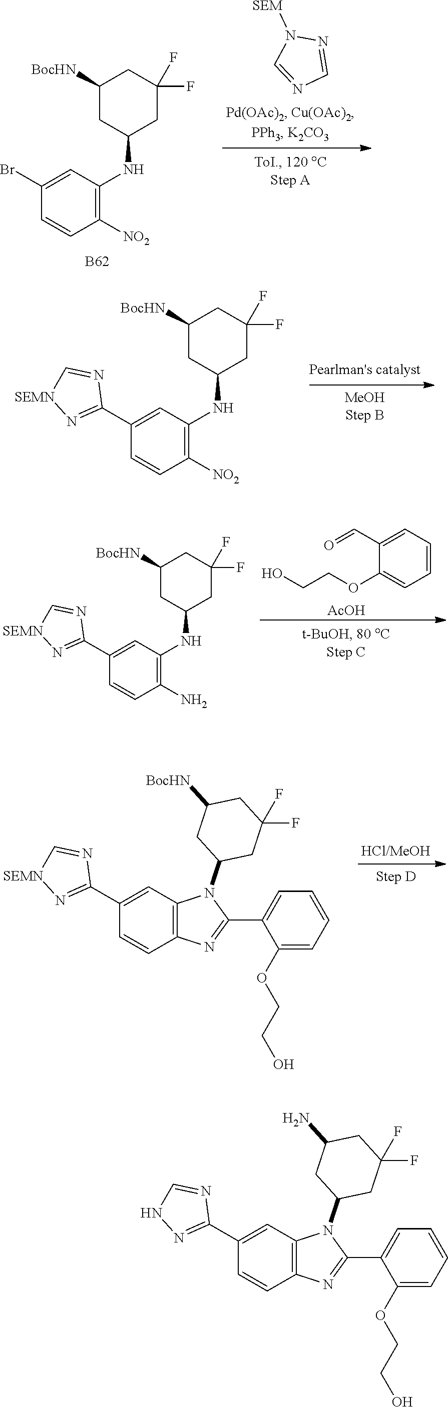

### Step A. Synthesis of tert-butyl (3,3-difluoro-5-((2-nitro-5-(1-((2-(trimethylsilyl)ethoxy)methyl)-1H-1,2,4-triazol-3-yl)phenyl)amino)cyclohexyl)carbamate

To a solution of tert-butyl (5-((5-bromo-2-nitrophenyl)amino)-3,3-difluorocyclohexyl)carbamate (B62) (165 mg, 366 μmol), K₂CO₃ ((101 mg, 733 μmol) and 1-((2-(trimethylsilyl)ethoxy)methyl)-1H-1,2,4-triazole (73 mg, 366 μmol) in toluene (8 mL) were added PPh₃ (48 mg, 183 μmol), Cu(OAc)₂ (6.66 mg, 37 μmol) and Pd(OAc)₂ (10 mg, 73 μmol). The reaction mixture was degassed under vacuum and purged with N₂ several times, and then heated at 125° C. under N₂ atmosphere for 3 hrs by microwave. After completion, the reaction mixture was concentrated to dryness. The residue was purified by flash column chromatography on silica gel to afford tert-butyl (3,3-difluoro-5-((2-nitro-5-(1-((2-(trimethylsilyl)ethoxy)methyl)-1H-1,2,4-triazol-3-yl)phenyl)amino)cyclohexyl)carbamate (160 mg, 77% yield). LC-MS: m/z 569 [M+H]⁺.

### Step B. Synthesis of rac-tert-butyl ((1R,5S)-5-((2-amino-5-(1-((2-(trimethylsilyl)ethoxy)methyl)-1H-1,2,4-triazol-3-yl)phenyl)amino)-3,3-difluorocyclohexyl)carbamate

To a solution of rac-tert-butyl ((1R,5S)-3,3-difluoro-5-((2-nitro-5-(1-((2-(trimethylsilyl)ethoxy)methyl)-1H-1,2,4-triazol-3-yl)phenyl)amino)cyclohexyl)carbamate (340 mg, 598 μmol) in MeOH (10 mL) was added Pearlman's catalyst (318 mg, 2.99 mmol) under nitrogen. The reaction mixture was degassed under vacuum and purged with H₂ several times, and then stirred at rt for 12 hrs. After completion, the reaction mixture was filtered through a pad of Celite®, and the filter cake was washed with MeOH (10 mL). The filtrates were concentrated to dryness to give tert-butyl (5-((2-amino-5-(1-((2-(trimethylsilyl)ethoxy)methyl)-1H-1,2,4-triazol-3-yl)phenyl)amino)-3,3-difluorocyclohexyl)carbamate (314 mg, 98% yield). LCMS: m/z 539 [M+H]⁺.

### Step C. Synthesis of tert-butyl (3,3-difluoro-5-(2-(2-(2-hydroxyethoxy)phenyl)-6-(1-((2-(trimethylsilyl)ethoxy)methyl)-1H-1,2,4-triazol-3-yl)-1H-benzol[d]imidazol-1-yl)cyclohexyl)carbamate

To a solution of tert-butyl (5-((2-amino-5-(1-((2-(trimethylsilyl)ethoxy)methyl)-1H-1,2,4-triazol-3-yl)phenyl)amino)-3,3-difluorocyclohexyl)carbamate (310 mg, 575 μmol) in t-BuOH (80 mL) were added 2-(2-hydroxyethoxy)benzaldehyde (191 mg, 1.15 mmol) and AcOH (35 mg, 575 μmol). The reaction mixture was stirred at 80° C. for 12 hrs and then concentrated to dryness. The residue was purified by flash column chromatography on silica gel to afford tert-butyl (3,3-difluoro-5-(2-(2-(2-hydroxyethoxy)phenyl)-6-(1-((2-(trimethylsilyl)ethoxy)methyl)-1H-1,2,4-triazol-3-yl)-1H-benzo[d]imidazol-1-yl)cyclohexyl)carbamate (300 mg, 76% yield). LC-MS: m/z 685 [M+H]⁺.

### Step D. Synthesis of 2-(2-(1-(5-amino-3,3-difluorocyclohexyl)-6-(1H-1,2,4-triazol-3-yl)-1H-benzo[d]imidazol-2-yl)phenoxy)ethan-1-ol

A solution of tert-butyl (3,3-difluoro-5-(2-(2-(2-hydroxyethoxy)phenyl)-6-(1-((2-(trimethylsilyl)ethoxy)methyl)-1H-1,2,4-triazol-3-yl)-1H-benzo[d]imidazol-1-yl)cyclohexyl)carbamate (280 mg, 409 μmol) in HCl/MeOH (3 mL) was stirred at rt for 2 hrs. After completion, the reaction mixture was concentrated to dryness to afford 2-(2-(1-(5-amino-3,3-difluorocyclohexyl)-6-(1H-1,2,4-triazol-3-yl)-1H-benzo[d]imidazol-2-yl)phenoxy)ethan-1-ol (186 mg, quant.), which was used without further purification. LC-MS: m/z 455 [M+H]⁺.

### Preparation of intermediate (1S,3R)-3-(7-fluoro-2-(2-fluorophenyl)-6-(1H-1,2,4-triazol-3-yl)-1H-benzo[d]imidazol-1-yl)cyclohexan-1-amine via General Scheme 9 (Methods A and E)

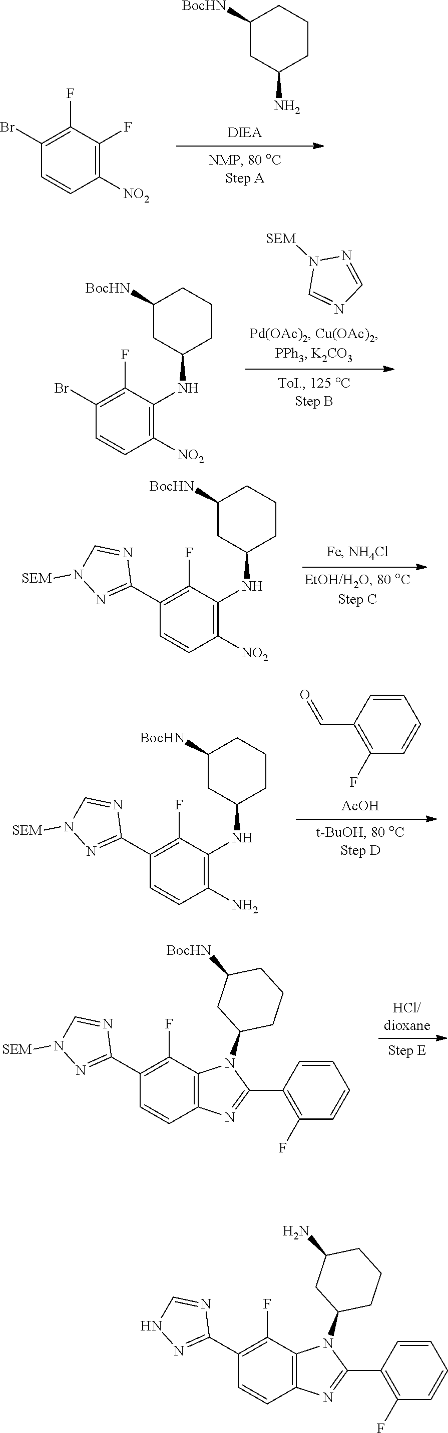

### Step A. Synthesis of tert-butyl ((1S,3R)-3-((3-bromo-2-fluoro-6-nitrophenyl)amino)cyclohexyl)carbamate

To a solution of 1-bromo-2,3-difluoro-4-nitrobenzene (1.0 g, 4.2 mmol) in NMP (10 mL) were added DIPEA (1.1 g, 8.4 mmol) and tert-butyl ((1S,3R)-3-aminocyclohexyl)carbamate (0.99 g, 4.6 mmol). The reaction mixture was stirred at 80° C. for 2 hrs and then cooled to rt. Water (15 mL) was added, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-((3-bromo-2-fluoro-6-nitrophenyl)amino)cyclohexyl)carbamate (1.3 g, 72% yield). LC-MS: m/z 432 [M+H]⁺.

### Step B. Synthesis of tert-butyl ((1S,3R)-3-((2-fluoro-6-nitro-3-(1-((2-(trimethylsilyl)ethoxy)methyl)-1H-1,2,4-triazol-3-yl)phenyl)amino)cyclohexyl)carbamate

To a solution of tert-butyl ((1S,3R)-3-((3-bromo-2-fluoro-6-nitrophenyl)amino)cyclohexyl)carbamate (1.57 g, 3.63 mmol), K₂CO₃ (1.00 g, 7.26 mmol) and 1-((2-(trimethylsilyl)ethoxy)methyl)-1H-1,2,4-triazole (941 mg, 4.72 mmol) in toluene (30 mL) were added PPh₃ (476 mg, 1.82 mmol), Cu(OAc)₂ (66 mg, 363 μmol) and Pd(OAc)₂ (163 mg, 726 μmol). The reaction mixture was degassed under vacuum and purged with N₂ for several times, then heated at 125° C. under N₂ atmosphere for 3 hrs. After completion, the reaction mixture was concentrated to dryness. The residue was purified by flash column chromatography on silica gel to afford tert-butyl ((1S,3R)-3-((2-fluoro-6-nitro-3-(1-((2-(trimethylsilyl)ethoxy)methyl)-1H-1,2,4-triazol-3-yl)phenyl)amino)cyclohexyl)carbamate (1.3 g, 65% yield). LC-MS: m/z 551 [M+H]⁺.

### Step C. Synthesis of tert-butyl ((1S,3R)-3-((6-amino-2-fluoro-3-(1-((2-(trimethylsilyl)ethoxy)methyl)-1H-1,2,4-triazol-3-yl)phenyl)amino)cyclohexyl)carbamate

To a solution of tert-butyl ((1S,3R)-3-((2-fluoro-6-nitro-3-(1-((2-(trimethylsilyl)ethoxy)methyl)-1H-1,2,4-triazol-3-yl)phenyl)amino)cyclohexyl)carbamate (50 mg, 91 μmol) and NH₄Cl (49 mg, 0.91 mmol) in EtOH/H₂O (3 mL/1 mL) was added Fe (51 mg, 0.91 mmol). The reaction mixture was then heated at 80° C. under N₂ atmosphere for 1 hr. After completion, the reaction mixture was cooled to rt, filtered through a pad of Celite®, and the filter cake was washed with MeOH (20 mL). The filtrate was concentrated to dryness. Water (15 mL) was added to the residue, and the aqueous portion extracted with EtOAc (15 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-((6-amino-2-fluoro-3-(1-((2-(trimethylsilyl)ethoxy)methyl)-1H-1,2,4-triazol-3-yl)phenyl)amino)cyclohexyl)carbamate (30 mg, 63% yield). LC-MS: m/z 521 [M+H]⁺.

### Step D. Synthesis of tert-butyl ((1S,3R)-3-(7-fluoro-2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate

To a mixture of tert-butyl ((1S,3R)-3-((6-amino-2-fluoro-3-(1-((2-(trimethylsilyl)ethoxy)methyl)-1H-1,2,4-triazol-3-yl)phenyl)amino)cyclohexyl)carbamate (400 mg, 768 μmol) in t-BuOH (15 mL) were added 2-fluorobenzaldehyde (191 mg, 1.54 mmol) and AcOH (0.6 mL). The reaction mixture was stirred at 80° C. for 16 hrs and then cooled to rt. After completion, water (15 mL) was added, and the aqueous portion extracted with EtOAc (15 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-(7-fluoro-2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate (400 mg, 83% yield). LC-MS: m/z 625 [M+H]⁺.

### Step E. Synthesis of (1S,3R)-3-(7-fluoro-2-(2-fluorophenyl)-6-(1H-1,2,4-triazol-3-yl)-1H-benzo[d]imidazol-1-yl)cyclohexan-1-amine

A solution of tert-butyl ((1S,3R)-3-(7-fluoro-2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate (300 mg, 480 μmol) in HCl/1,4-dioxane (10 mL, 4M) was stirred at rt for 1 hr. After completion, the reaction mixture was concentrated to dryness to afford (1S,3R)-3-(7-fluoro-2-(2-fluorophenyl)-6-(1H-1,2,4-triazol-3-yl)-1H-benzo[d]imidazol-1-yl)cyclohexan-1-amine (300 mg), which was used without further purification. LC-MS: m/z 395 [M+H]⁺.

### Preparation of intermediate (1R,2S,6R)-2-amino-6-(2-(2-fluorophenyl)-6-(1H-1,2,4-triazol-3-yl)-1H-benzo[d]imidazol-1-yl)cyclohexan-1-ol via General Scheme 9 (Methods A and E)

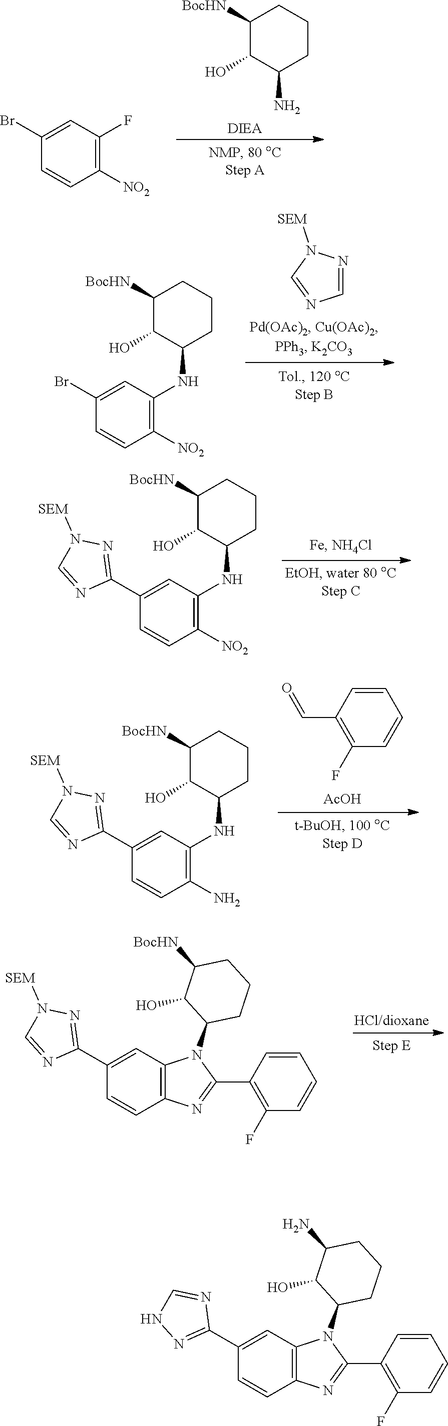

### Step A. Synthesis of tert-butyl ((1S,2S,3R)-3-((5-bromo-2-nitrophenyl)amino)-2-hydroxycyclohexyl)carbamate

A mixture of 4-bromo-2-fluoro-1-nitrobenzene (3.0 g, 14 mmol), tert-butyl ((1S,2S,3R)-3-amino-2-hydroxycyclohexyl)carbamate (3.5 g, 15 mmol) and DIEA (3.5 g, 4.8 mL, 27 mmol) in NMP (30 mL) was stirred at 80° C. for 2 hrs and then allowed to cool. After completion, the reaction mixture was poured into ice-water (20 mL), and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to give tert-butyl ((1S,2S,3R)-3-((5-bromo-2-nitrophenyl)amino)-2-hydroxycyclohexyl)carbamate (5.28 g, 90% yield). LC-MS: m/z 430 [M+H]⁺.

### Step B. Synthesis of tert-butyl ((1S,2S,3R)-2-hydroxy-3-((2-nitro-5-(1-((2-(trimethylsilyl)ethoxy)methyl)-1H-1,2,4-triazol-3-yl)phenyl)amino)cyclohexyl)carbamate

To a solution of tert-butyl ((1S,2S,3R)-3-((5-bromo-2-nitrophenyl)amino)-2-hydroxycyclohexyl)carbamate (1.2 g, 2.8 mmol), K₂CO₃ (0.77 g, 5.6 mmol) and 1-((2-(trimethylsilyl)ethoxy)methyl)-1H-1,2,4-triazole (0.67 g, 3.3 mmol) in toluene (10 mL) were added PPh₃ (0.37 g, 1.4 mmol), Cu(OAc)₂ (51 mg, 0.28 mmol), and Pd(OAc)₂ (0.13 g, 0.56 mmol). The reaction mixture was degassed under vacuum and purged with N₂ several times, then heated at 120° C. under N₂ atmosphere for 3 hrs. After completion, the reaction mixture was concentrated to dryness. The residue was purified by flash column chromatography on silica gel to afford tert-butyl ((1S,2S,3R)-2-hydroxy-3-((2-nitro-5-(1-((2-(trimethylsilyl)ethoxy)methyl)-1H-1,2,4-triazol-3-yl)phenyl)amino)cyclohexyl)carbamate (1.2 g, 78% yield). LC-MS: m/z 549 [M+H]⁺.

### Step C. Synthesis of tert-butyl ((1S,2S,3R)-3-((2-amino-5-(1-((2-(trimethylsilyl)ethoxy)methyl)-1H-1,2,4-triazol-3-yl)phenyl)amino)-2-hydroxycyclohexyl)carbamate

To a solution of tert-butyl ((1S,2S,3R)-2-hydroxy-3-((2-nitro-5-(1-((2-(trimethylsilyl)ethoxy)methyl)-1H-1,2,4-triazol-3-yl)phenyl)amino)cyclohexyl)carbamate (1.2 g, 2.2 mmol) and NH₄Cl (0.58 g, 11 mmol) in EtOH/H₂O (15 mL/5 mL) was added Fe (0.61 g, 11 mmol). The reaction mixture was then heated at 80° C. under N₂ atmosphere for 0.5 hrs. After completion, the reaction mixture was cooled to rt and filtered through a pad of Celite®. The filter cake was washed with MeOH (20 mL), and the filtrate concentrated under reduced pressure. Water (15 mL) was added to the residue, and the aqueous portion extracted with EtOAc (50 mL×3). The combined organic layers were washed with brine (10 mL), dried over Na₂SO₄, filtered, and concentrated to dryness. The residue was purified by flash column chromatography on silica gel to afford tert-butyl ((1S,2S,3R)-3-((2-amino-5-(1-((2-(trimethylsilyl)ethoxy)methyl)-1H-1,2,4-triazol-3-yl)phenyl)amino)-2-hydroxycyclohexyl)carbamate (1.0 g, 79% yield). LC-MS: m/z 519 [M+H]⁺.

### Step D. Synthesis of tert-butyl ((1S,2R,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate

To a solution of tert-butyl ((1S,2S,3R)-3-((2-amino-5-(1-((2-(trimethylsilyl)ethoxy)methyl)-1H-1,2,4-triazol-3-yl)phenyl)amino)-2-hydroxycyclohexyl)carbamate (500 mg, 964 μmol) in t-BuOH (8 mL) were added 2-fluorobenzaldehyde (179 mg, 1.45 mmol) and AcOH (0.6 mL). The reaction mixture was stirred at 100° C. for 16 hrs and then cooled to rt. After completion, water (15 mL) was added, and the aqueous portion extracted with EtOAc (20 mL×3). The combined organic layers were washed with brine (10 mL), dried over Na₂SO₄, filtered, and concentrated to dryness. The residue was purified by flash column chromatography on silica gel to afford tert-butyl ((1S,2R,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (200 mg, 33% yield). LC-MS: m/z 623 [M+H]⁺.

### Step E. Synthesis of (1R,2S,6R)-2-amino-6-(2-(2-fluorophenyl)-6-(1H-1,2,4-triazol-3-yl)-1H-benzo[d]imidazol-1-yl)cyclohexan-1-ol

A solution of tert-butyl ((1S,2R,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (200 mg, 321 μmol) in HCl/dioxane (10 mL, 4M) was stirred at rt for 2 hrs. After completion, the reaction mixture was concentrated to dryness to afford (1R,2S,6R)-2-amino-6-(2-(2-fluorophenyl)-6-(1H-1,2,4-triazol-3-yl)-1H-benzo[d]imidazol-1-yl)cyclohexan-1-ol (100 mg), which was used without further purification. LC-MS: m/z 393 [M+H]⁺.

### Preparation of Example 15 via General Scheme 1A

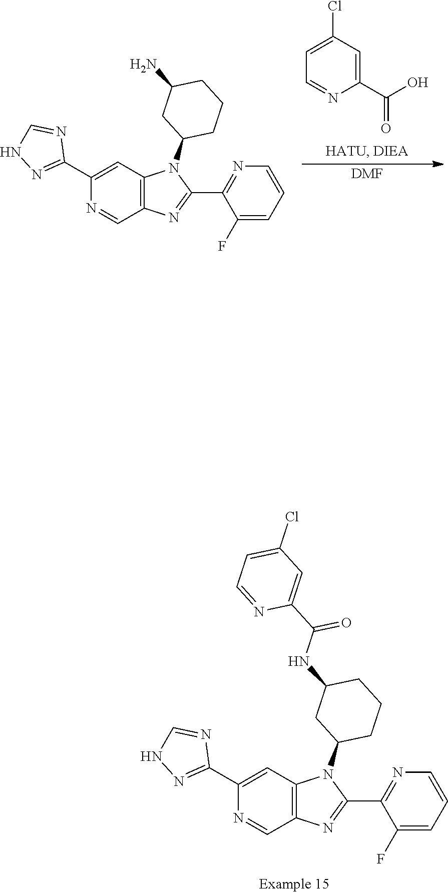

### Synthesis of 4-chloro-N-((1S,3R)-3-(2-(3-fluoropyridin-2-yl)-6-(1H-1,2,4-triazol-3-yl)-1H-imidazo[4,5-c]pyridin-1-yl)cyclohexyl)picolinamide (Example 15)

To a solution of (1S,3R)-3-(2-(3-fluoropyridin-2-yl)-6-(1H-1,2,4-triazol-3-yl)-1H-imidazo[4,5-c]pyridin-1-yl)cyclohexan-1-amine (70 mg, 0.18 mmol) in DMF (6 mL) were added 4-chloropicolinic acid (44 mg, 0.28 mmol), HATU (0.14 g, 0.37 mmol) and DIEA (96 mg, 0.74 mmol). The reaction mixture was stirred at rt for 16 hrs. Water (10 mL) was added, and the aqueous portion extracted with EtOAc (15 mL×3). The combined organic layers were combined and washed with brine (10 mL), dried over Na₂SO₄, filtered, and concentrated to dryness. The residue was purified by prep-HPLC to afford Example 15 (67 mg, 70% yield). ¹H NMR (400 MHz, DMSO-d₆) δ 14.65 (s, 1H), 9.17 (s, 1H), 9.05 (d, 1H), 8.71 (d, 1H), 8.61 (d, 1H), 8.56 (s, 1H), 8.16 (s, 1H), 8.08 (t, 1H), 8.00 (d, 1H), 7.84-7.78 (m, 1H), 7.77-7.73 (m, 1H), 4.80-4.67 (m, 1H), 4.07-3.95 (m, 1H), 2.70-2.56 (m, 1H), 2.19-1.97 (m, 3H), 1.92-1.77 (m, 2H), 1.72-1.58 (m, 1H), 1.56-1.40 (m, 1H); LC-MS: m/z 518.2 [M+H]⁺; >99% ee (based on enantiopurity of chiral intermediate).

### Preparation of Example 21 via General Schemes 1A and 2D

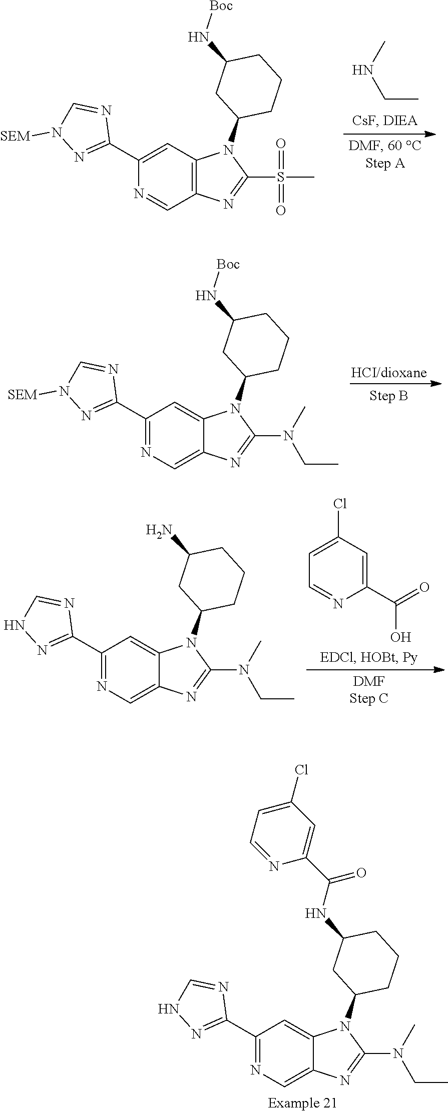

### Step A. Synthesis of tert-butyl ((1S,3R)-3-(2-(ethyl(methyl)amino)-6-(1-((2-(trimethylsilyl)ethoxy)methyl)-1H-1,2,4-triazol-3-yl)-1H-imidazo[4,5-c]pyridin-1-yl)cyclohexyl)carbamate

A mixture of tert-butyl ((1S,3R)-3-(2-(methylsulfonyl)-6-(1-((2-(trimethylsilyl)ethoxy)methyl)-1H-1,2,4-triazol-3-yl)-1H-imidazo[4,5-c]pyridin-1-yl)cyclohexyl)carbamate (210 mg, 355 μmol), N-methylethanamine (63 mg, 1.06 mmol), DIEA (138 mg, 1.06 mmol) and CsF (162 mg, 1.06 mmol) in DMF (3 mL) was stirred at 60° C. for 12 hrs and then allowed to cool. Water (15 mL) was added, and the aqueous portion extracted with EtOAc (15 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-(2-(ethyl(methyl)amino)-6-(1-((2-(trimethylsilyl)ethoxy)methyl)-1H-1,2,4-triazol-3-yl)-1H-imidazo[4,5-c]pyridin-1-yl)cyclohexyl)carbamate (180 mg, 89% yield). LC-MS: m/z 571 [M+H]⁺.

### Step B. Synthesis of 1-((1R,3S)-3-aminocyclohexyl)-N-ethyl-N-methyl-6-(1H-1,2,4-triazol-3-yl)-1H-imidazo[4,5-c]pyridin-2-amine

A solution of tert-butyl ((1S,3R)-3-(2-(ethyl(methyl)amino)-6-(1-((2-(trimethylsilyl)ethoxy)methyl)-1H-1,2,4-triazol-3-yl)-1H-imidazo[4,5-c]pyridin-1-yl)cyclohexyl)carbamate (180 mg, 315 μmol) in HCl/Dioxane (4 mL, 4 M) was stirred at rt for 1 hr. After completion, the reaction mixture was concentrated to dryness to afford 1-((1R,3S)-3-aminocyclohexyl)-N-ethyl-N-methyl-6-(1H-1,2,4-triazol-3-yl)-1H-imidazo[4,5-c]pyridin-2-amine (107 mg), which was used directly without further purification. LC-MS: m/z 341 [M+H]⁺.

### Step C. Synthesis of 4-chloro-N-((1S,3R)-3-(2-(ethyl(methyl)amino)-6-(1H-1,2,4-triazol-3-yl)-1H-imidazo[4,5-c]pyridin-1-yl)cyclohexyl)picolinamide (Example 21)

To a solution of 1-((1R,3S)-3-aminocyclohexyl)-N-ethyl-N-methyl-6-(1H-1,2,4-triazol-3-yl)-1H-imidazo[4,5-c]pyridin-2-amine (54 mg, 0.16 mmol) and 4-chloropicolinic acid (37 mg, 0.24 mmol) in DMF (4 mL) were added HOBt (43 mg, 0.32 mmol), EDCI (61 mg, 0.32 mmol) and pyridine (50 mg, 0.63 mmol). The reaction mixture was stirred at rt for 2 hrs and then purified by Prep-HPLC to afford Example 21 (24 mg, 32% yield). ¹H NMR (400 MHz, DMSO-d₆) δ 14.50 (s, 1H), 9.09 (d, 1H), 8.76 (s, 1H), 8.61 (d, 1H), 8.22 (s, 1H), 8.04 (s, 1H), 8.01 (d, 1H), 7.77-7.72 (m, 1H), 4.41-4.26 (m, 1H), 4.20-4.06 (m, 1H), 3.26 (q, 2H), 2.93 (s, 3H), 2.65-2.53 (m, 1H), 2.15-1.82 (m, 5H), 1.75-1.51 (m, 2H), 1.21 (t, 3H); LC-MS: m/z 480.5 [M+H]⁺; >99% ee (based on enantiopurity of chiral intermediate).

### Preparation of Example 82 via General Scheme 1A

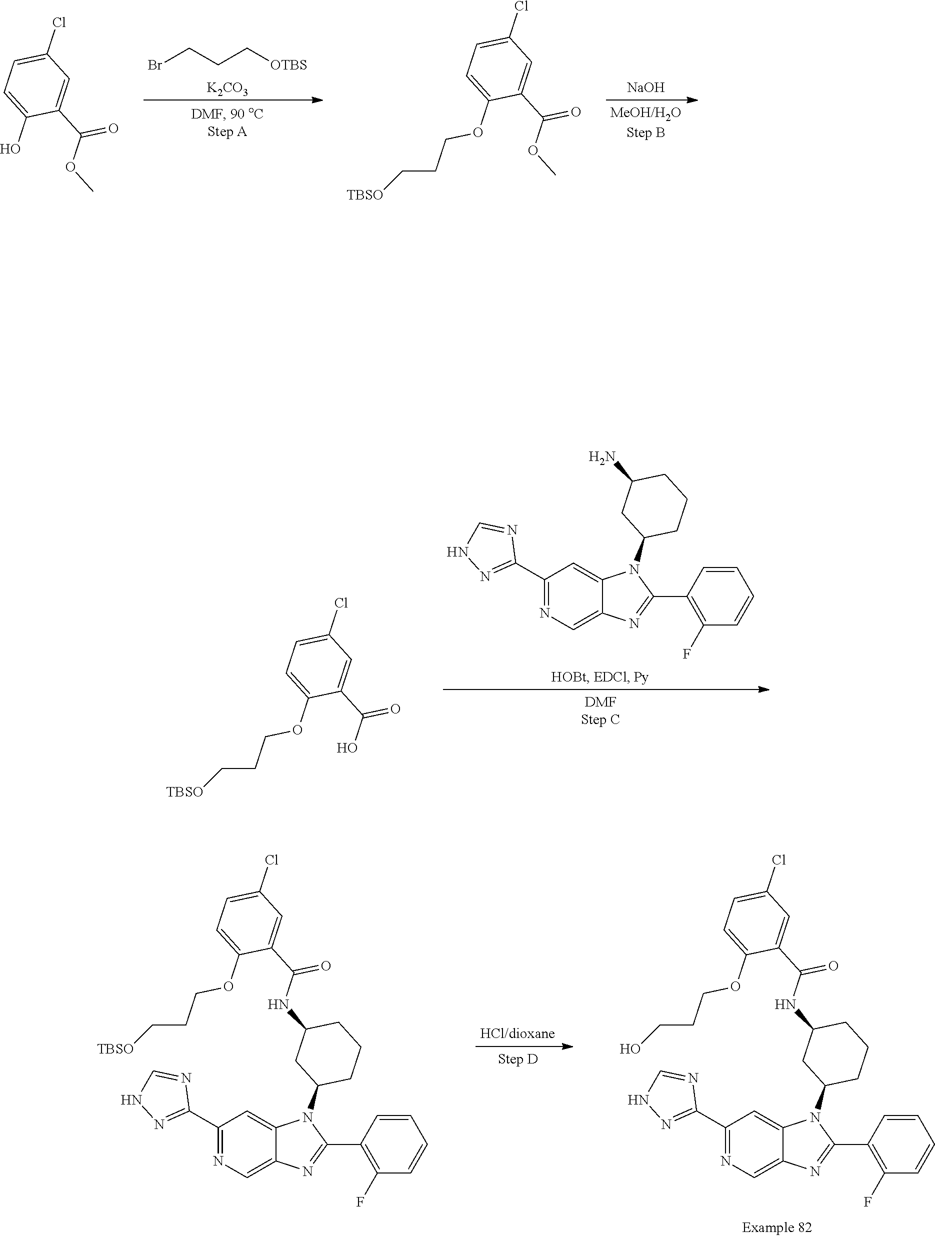

### Step A. Synthesis of methyl 2-(3-((tert-butyldimethylsilyl)oxy)propoxy)-5-chlorobenzoate

To solution of methyl 5-chloro-2-hydroxybenzoate (1 g, 5.38 mmol) in DMF (12 mL) were added K₂CO₃ (1 g, 10.8 mmol) and (3-bromopropoxy)(tert-butyl)dimethylsilane (2 g, 7 mmol). The reaction mixture was stirred at 90° C. for 3 hrs. After completion, the reaction mixture was cooled to rt, and water (20 mL) was added to the reaction mixture. The aqueous portion was extracted with EtOAc (30 mL×3). The combined organic layers were washed with brine (30 mL), dried over Na₂SO₄, filtered, and concentrated to dryness. The residue was purified by flash column chromatography on silica gel to afford methyl 2-(3-((tert-butyldimethylsilyl)oxy)propoxy)-5-chlorobenzoate (1.5 g, 80% yield). LCMS: m/z 359 [M+H]⁺.

### Step B. Synthesis of 2-(3-((tert-butyldimethylsilyl)oxy)propoxy)-5-chlorobenzoic acid

To a solution of methyl 2-(3-((tert-butyldimethylsilyl)oxy)propoxy)-5-chlorobenzoate (1.3 g, 3.6 mmol) in MeOH (20 mL) and H₂O (6 mL) was added NaOH (290 mg, 7.2 mmol). The reaction mixture was stirred at rt for 16 hrs. After completion, the reaction mixture was cooled down in an ice bath, then HCl (aq., 1 N) was added until the pH was adjusted to pH 5-6. The aqueous portion was extracted with EtOAc (20 mL×3), and the combined organic layers were washed with brine (20 mL), dried over Na₂SO₄, filtered, and concentrated to dryness to afford 2-(3-((tert-butyldimethylsilyl)oxy)propoxy)-5-chlorobenzoic acid (610 mg, 49% yield). LCMS: m/z 345 [M+H]⁺.

### Step C. Synthesis of 2-(3-((tert-butyldimethylsilyl)oxy)propoxy)-5-chloro-N-((1S,3R)-3-(2-(2-fluorophenyl)-6-(1H-1,2,4-triazol-3-yl)-1H-imidazo[4,5-c]pyridin-1-yl)cyclohexyl)benzamide

To a solution of (1S,3R)-3-(2-(2-fluorophenyl)-6-(1H-1,2,4-triazol-3-yl)-1H-imidazo[4,5-c]pyridin-1-yl)cyclohexan-1-amine (150 mg, 397 μmol) and 2-(3-((tert-butyldimethylsilyl)oxy)propoxy)-5-chlorobenzoic acid (137 mg, 397 μmol) in DMF (5 mL) were added pyridine (189 mg, 2.38 mmol), HOBt (81 mg, 596 μmol) and EDCI (152 mg, 795 mol). The reaction mixture was stirred at rt for 16 hrs. After completion, the reaction mixture was purified by C18 column to give 2-(3-((tert-butyldimethylsilyl)oxy)propoxy)-5-chloro-N-((1S,3R)-3-(2-(2-fluorophenyl)-6-(1H-1,2,4-triazol-3-yl)-1H-imidazo[4,5-c]pyridin-1-yl)cyclohexyl)benzamide (250 mg, 89% yield). LCMS: m/z 704 [M+H]⁺.

### Step D. Synthesis of 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-(3-hydroxypropoxy)benzamide

A solution of 2-(3-((tert-butyldimethylsilyl)oxy)propoxy)-5-chloro-N-((1S,3R)-3-(2-(2-fluorophenyl)-6-(1H-1,2,4-triazol-3-yl)-1H-imidazo[4,5-c]pyridin-1-yl)cyclohexyl)benzamide (220 mg, 312 μmol) in HCl/1,4-Dioxane (5 mL, 4M) was stirred at rt for 1 hr. After completion, the reaction mixture was concentrated to dryness to give Example 82 (100 mg, 54% yield). ¹H NMR (400 MHz, DMSO-d₆) δ 14.66 (s, 1H), 9.14 (s, 1H), 8.50 (s, 1H), 8.31 (d, 1H), 8.17 (s, 1H), 7.81-7.70 (m, 2H), 7.61-7.42 (m, 4H), 7.13 (d, 1H), 4.76-4.57 (m, 1H), 4.35-4.20 (m, 1H), 4.11 (t, 2H), 4.03-3.88 (m, 1H), 3.55 (t, 2H), 2.35-2.23 (m, 1H), 2.21-2.07 (m, 2H), 2.01-1.84 (m, 5H), 1.53-1.38 (m, 2H); LC-MS: m/z 590.5 [M+H]⁺; >99% ee (based on enantiopurity of chiral intermediate).

### Preparation of Example 90 according to General Scheme 1A

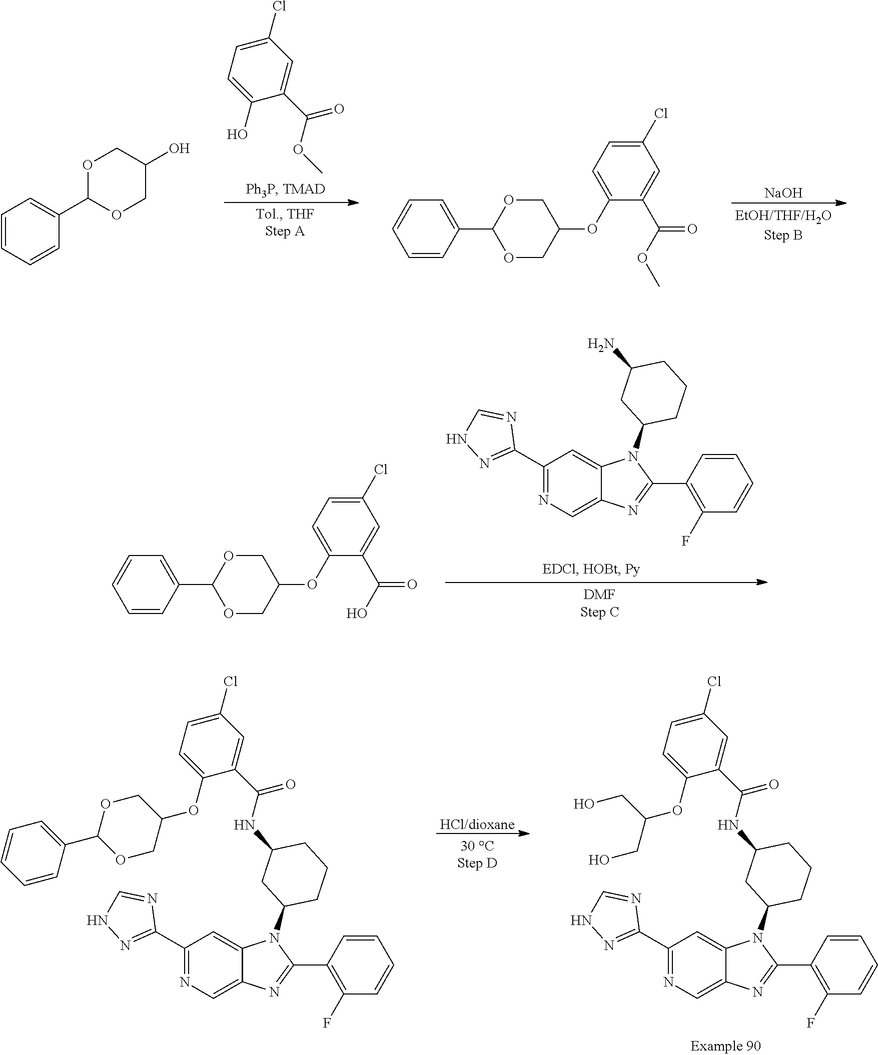

### Step A. Synthesis of methyl 5-chloro-2-((2-phenyl-1,3-dioxan-5-yl)oxy)benzoate

To a solution of 2-phenyl-1,3-dioxan-5-ol (400 mg, 2.22 mmol) in toluene (2 mL) and THF (2 mL) was added methyl 5-chloro-2-hydroxybenzoate (414 mg, 2.22 mmol), and then TMAD (382 mg, 2.22 mmol) and PPh₃ (582 mg, 2.22 mmol) were added. The reaction mixture was stirred at rt for 16 hrs. After completion, water (20 mL) was added, and the aqueous portion extracted with EtOAc (10 mL×3). The combined organic layers were washed with brine (10 mL), dried over Na₂SO₄, filtered, and concentrated to dryness. The residue was purified by flash column chromatography on silica gel to afford methyl 5-chloro-2-((2-phenyl-1,3-dioxan-5-yl)oxy)benzoate (380 mg, 49% yield). LCMS: m/z 473 [M+H]⁺.

### Step B. Synthesis of 5-chloro-2-((2-phenyl-1,3-dioxan-5-yl)oxy)benzoic acid

To a solution of methyl 5-chloro-2-((2-phenyl-1,3-dioxan-5-yl)oxy)benzoate (200 mg, 0.6 mmol) in EtOH (5 mL) and THF (2 mL) was added a solution of NaOH (24 mg, 0.6 mmol) in H₂O (1 mL). The reaction mixture was stirred at rt for 16 hrs. After completion, the reaction mixture was cooled in an ice bath, and then HCl (aq., 1 N) was added until the pH was adjusted to pH=2-3. The aqueous portion was extracted with EtOAc (10 mL×3), and the combined organic layers were washed with brine (10 mL), dried over Na₂SO₄, filtered, and concentrated to dryness to afford 5-chloro-2-((2-phenyl-1,3-dioxan-5-yl)oxy)benzoic acid (300 mg, quant.). LCMS: m/z 335 [M+H]⁺.

### Step C. Synthesis of 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-((2-phenyl-1,3-dioxan-5-yl)oxy)benzamide

To a solution of (1S,3R)-3-(2-(2-fluorophenyl)-6-(1H-1,2,4-triazol-3-yl)-1H-imidazo[4,5-c]pyridin-1-yl)cyclohexan-1-amine (180 mg, 477 μmol) and 5-chloro-2-((2-phenyl-1,3-dioxan-5-yl)oxy)benzoic acid (319 mg, 954 μmol) in DMF (5 mL) were added pyridine (189 mg, 2.38 mmol), HOBt (37 mg, 238 μmol) and EDCI (46 mg, 238 μmol). The reaction mixture was stirred at rt for 2 hrs. After completion, the reaction mixture was purified by C18 column to give 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-((2-phenyl-1,3-dioxan-5-yl)oxy)benzamide (160 mg, 48% yield). LCMS: m/z 694 [M+H]⁺.

### Step D. Synthesis of 5-chloro-2-((1,3-dihydroxypropan-2-yl)oxy)-N-((1S,3R)-3-(2-(2-fluorophenyl)-6-(1H-1,2,4-triazol-3-yl)-1H-imidazo[4,5-c]pyridin-1-yl)cyclohexyl)benzamide

A solution of 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-((2-phenyl-1,3-dioxan-5-yl)oxy)benzamide (150 mg, 216 μmol) in HCl/1,4-Dioxane (5 mL, 4M) was stirred at 30° C. for 2 hrs. After completion, the reaction mixture was concentrated to dryness to give Example 90 (100 mg, 80% yield). ¹H NMR (400 MHz, DMSO-d₆) δ 14.72 (s, 1H), 9.13 (s, 1H), 8.61 (d, 1H), 8.48 (s, 1H), 8.17 (s, 1H), 7.74 (t, 2H), 7.63 (d, 1H), 7.57-7.45 (m, 3H), 7.32 (d, 1H), 5.45-5.09 (m, 2H), 4.47-4.36 (m, 1H), 4.38-4.22 (m, 1H), 4.02-3.86 (m, 1H), 3.75-3.55 (m, 4H), 2.33-2.18 (m, 2H), 2.08-1.84 (m, 4H), 1.53-1.38 (m, 2H); LC-MS: m/z 606.3 [M+H]⁺; >99% ee (based on enantiopurity of chiral intermediate).

### Synthesis of Example 95 and 96 Via General Schemes 1A and 2A (Method G)

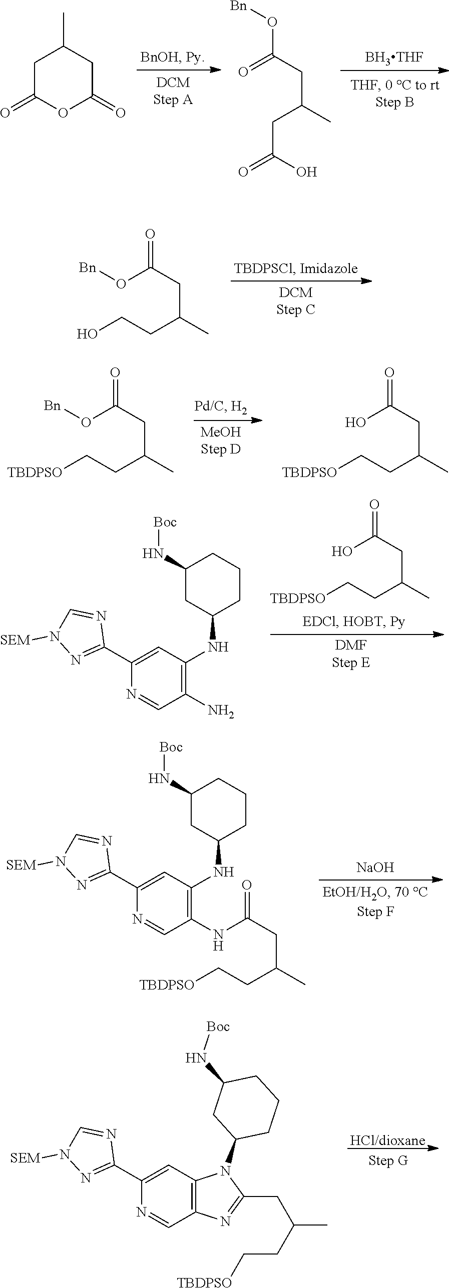

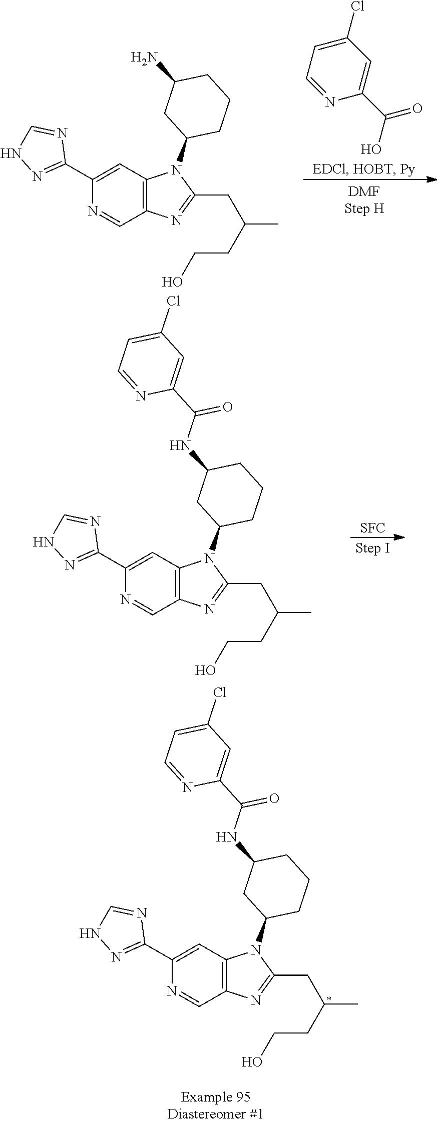

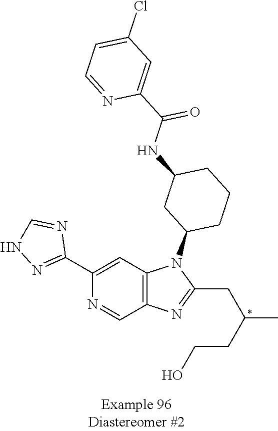 [SMILES: CC(CCO)Cc1nc2cnc(-c3nc[nH]n3)cc2n1[C@@H]1CCC[C@H](NC(=O)c2cc(Cl)ccn2)C1]

### Step A. Synthesis of 5-(benzyloxy)-3-methyl-5-oxopentanoic acid

To a solution of 4-methyldihydro-2H-pyran-2,6(3H)-dione (1 g, 7.81 mmol) and Py (3.2 mL, 39.0 mmol) in DCM (10 mL) was added phenylmethanol (1.1 g, 10.2 mmol). The reaction mixture was stirred at rt for 72 hrs. Water (10 mL) was added to the reaction mixture, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford 5-(benzyloxy)-3-methyl-5-oxopentanoic acid (1.8 g, 90% yield). LC-MS: m/z 237 [M+H]⁺.

### Step B. Synthesis of benzyl 5-hydroxy-3-methylpentanoate

To a solution of 5-(benzyloxy)-3-methyl-5-oxopentanoic acid (3 g, 12.7 mmol) in THF (10 mL) was added BH₃/THF (2.5 mL, 25.4 mmol). The reaction mixture was stirred at rt for 5 hrs. Water (10 mL) was added to the reaction mixture, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford benzyl 5-hydroxy-3-methylpentanoate (1.1 g, 39% yield). LC-MS: m/z 223 [M+H]⁺.

### Step C. Synthesis of benzyl 5-((tert-butyldiphenylsilyl)oxy)-3-methylpentanoate

To a solution of benzyl 5-hydroxy-3-methylpentanoate (200 mg, 900 μmol) in DCM (10 mL) and imidazole (123 mg, 1.80 mmol) was added tert-butylchlorodiphenylsilane (297 mg, 1.08 mmol). The reaction mixture was stirred at rt for 12 hrs. Water (10 mL) was added, and the aqueous portion extracted with DCM (20 mL×3). The combined organic layers were washed with brine (10 mL), dried over Na₂SO₄, filtered, and concentrated to dryness. The residue was purified by flash column chromatography on silica gel to afford benzyl 5-((tert-butyldiphenylsilyl)oxy)-3-methylpentanoate (100 mg, 24% yield). LC-MS: m/z 461 [M+H]⁺.

### Step D. Synthesis of 5-((tert-butyldiphenylsilyl)oxy)-3-methylpentanoic acid

To a solution of benzyl 5-((tert-butyldiphenylsilyl)oxy)-3-methylpentanoate (100 mg, 217 μmol) in MeOH (10 mL) was added Pd/C (10 mg) under nitrogen. The reaction mixture was degassed under vacuum, purged with H₂ several times, and stirred at rt for 12 hrs. After completion, the reaction mixture was filtered through a pad of Celite®, and the filter cake was washed with MeOH (10 mL). The filtrates were concentrated to dryness to give 5-((tert-butyldiphenylsilyl)oxy)-3-methylpentanoic acid (80 mg, quant.). LCMS: m/z 371 [M+H]⁺.

### Step E. Synthesis of tert-butyl ((1S,3R)-3-((5-(5-((tert-butyldiphenylsilyl)oxy)-3-methylpentanamido)-2-(1-((2-(trimethylsilyl)ethoxy)methyl)-1H-1,2,4-triazol-3-yl)pyridin-4-yl)amino)cyclohexyl)carbamate

To a solution of 5-((tert-butyldiphenylsilyl)oxy)-3-methylpentanoic acid (353 mg, 953 μmol) and tert-butyl ((1S,3R)-3-((5-amino-2-(1-((2-(trimethylsilyl)ethoxy)methyl)-1H-1,2,4-triazol-3-yl)pyridin-4-yl)amino)cyclohexyl)carbamate (400 mg, 794 μmol) in DMF (10 mL) were added EDCI (152 mg, 794 μmol), HOBt (107 mg, 794 μmol) and pyridine. (188 mg, 2.38 mmol). The reaction mixture was stirred at rt for 5 hrs. After completion, water (10 mL) was added to the reaction mixture, and the aqueous portion extracted with DCM (20 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-((5-(5-((tert-butyldiphenylsilyl)oxy)-3-methylpentanamido)-2-(1-((2-(trimethylsilyl)ethoxy)methyl)-1H-1,2,4-triazol-3-yl)pyridin-4-yl)amino)cyclohexyl)carbamate (300 mg, 44% yield). LC-MS: m/z 856 [M+H]⁺.

### Step F. Synthesis of tert-butyl ((1S,3R)-3-(2-(4-((tert-butyldiphenylsilyl)oxy)-2-methylbutyl)-6-(1-((2-(trimethylsilyl)ethoxy)methyl)-1H-1,2,4-triazol-3-yl)-1H-imidazo[4,5-c]pyridin-1-yl)cyclohexyl)carbamate

To a solution of tert-butyl ((1S,3R)-3-((5-(5-((tert-butyldiphenylsilyl)oxy)-3-methylpentanamido)-2-(1-((2-(trimethylsilyl)ethoxy)methyl)-1H-1,2,4-triazol-3-yl)pyridin-4-yl)amino)cyclohexyl)carbamate (200 mg, 234 μmol) in EtOH/H₂O (2 mL/2 mL) was added NaOH (28.0 mg, 701 μmol), and the reaction mixture was stirred at 70° C. for 18 hrs. After completion, the reaction mixture was cooled to rt, and water (10 mL) was added to the reaction mixture. The aqueous portion was extracted with DCM (20 mL×3). The combined organic layers were washed with brine (10 mL), dried over Na₂SO₄, filtered, and concentrated to dryness. The residue was purified by flash column chromatography on silica gel to afford tert-butyl ((1S,3R)-3-(2-(4-((tert-butyldiphenylsilyl)oxy)-2-methylbutyl)-6-(1-((2-(trimethylsilyl)ethoxy)methyl)-1H-1,2,4-triazol-3-yl)-1H-imidazo[4,5-c]pyridin-1-yl)cyclohexyl)carbamate (60 mg, 43% yield). LC-MS: m/z 838 [M+H]⁺.

### Step G. Synthesis of 4-(1-((1R,3S)-3-aminocyclohexyl)-6-(1H-1,2,4-triazol-3-yl)-1H-imidazo[4,5-c]pyridin-2-yl)-3-methylbutan-1-ol

A solution of tert-butyl ((1S,3R)-3-(2-(4-((tert-butyldiphenylsilyl)oxy)-2-methylbutyl)-6-(1-((2-(trimethylsilyl)ethoxy)methyl)-1H-1,2,4-triazol-3-yl)-1H-imidazo[4,5-c]pyridin-1-yl)cyclohexyl)carbamate (100 mg, 167 μmol) in HCl/1,4-dioxane (2 mL) was stirred at rt for 1 hr. After completion, the reaction mixture was concentrated to dryness to afford 4-(1-((1R,3S)-3-aminocyclohexyl)-6-(1H-1,2,4-triazol-3-yl)-1H-imidazo[4,5-c]pyridin-2-yl)-3-methylbutan-1-ol (80 mg, quant.), which was used without further purification. LC-MS: m/z 370 [M+H]⁺.

### Step H. Synthesis of 4-chloro-N-((1S,3R)-3-(2-(4-hydroxy-2-methylbutyl)-6-(1H-1,2,4-triazol-3-yl)-1H-imidazo[4,5-c]pyridin-1-yl)cyclohexyl)picolinamide

To a solution of 4-chloropicolinic acid (51.2 mg, 325 μmol) and 4-(1-((1R,3S)-3-aminocyclohexyl)-6-(1H-1,2,4-triazol-3-yl)-1H-imidazo[4,5-c]pyridin-2-yl)-3-methylbutan-1-ol (100 mg, 271 μmol) in DMF (10 mL) were added EDCI (104 mg, 541 mol), HOBt (73 mg, 541 μmol) and pyridine (77 mg, 975 μmol), and the reaction was stirred at rt for 5 hrs. After completion, the reaction mixture was purified by prep-HPLC to afford 4-chloro-N-((1S,3R)-3-(2-(4-hydroxy-2-methylbutyl)-6-(1H-1,2,4-triazol-3-yl)-1H-imidazo[4,5-c]pyridin-1-yl)cyclohexyl)picolinamide (20 mg, 15% yield). ¹H NMR (400 MHz, DMSO-d₆) δ 14.55 (s, 1H), 9.08 (d, 1H), 8.95 (s, 1H), 8.61 (d, 1H), 8.40 (s, 1H), 8.25 (s, 1H), 8.03 (d, 1H), 7.76 (dd, 1H), 4.77-4.59 (m, 1H), 4.55-4.33 (m, 1H), 4.34-4.15 (m, 1H), 3.56-3.47 (m, 2H), 3.14-2.98 (m, 1H), 2.95-2.77 (m, 1H), 2.73-2.56 (m, 1H), 2.30-2.16 (m, 1H), 2.15-1.97 (m, 2H), 1.96-1.81 (m, 3H), 1.77-1.53 (m, 3H), 1.52-1.34 (m, 1H), 0.98 (t, 3H); LC-MS: m/z 509.2 [M+H]⁺.

### Step I. Synthesis of Examples 95 and 96

4-chloro-N-((1S,3R)-3-(2-(4-hydroxy-2-methylbutyl)-6-(1H-1,2,4-triazol-3-yl)-1H-imidazo[4,5-c]pyridin-1-yl)cyclohexyl)picolinamide (40 mg) was separated by chiral SFC to give Diastereomer #1 (15 mg, 38% yield, Rt=4.820 min) and Diastereomer #2 (18 mg, 45% yield, Rt=6.889 min). SFC Method: (column: ChiralPak C-IG (250*30 mm, 5 μm), mobile phase: [0.1% NH₃H₂O EtOH]; %: 35%-35%,min).

Diastereomer #1 (Example 95): ¹H NMR (400 MHz, DMSO-d₆) δ 14.48 (s, 1H), 9.08 (d, 1H), 8.95 (s, 1H), 8.61 (d, 1H), 8.40 (s, 1H), 8.15 (s, 1H), 8.03 (s, 1H), 7.79-7.70 (m, 1H), 4.76-4.56 (m, 1H), 4.51-4.39 (m, 1H), 4.31-4.15 (m, 1H), 3.61-3.44 (m, 2H), 3.08-2.97 (m, 1H), 2.93-2.81 (m, 1H), 2.73-2.57 (m, 1H), 2.28-2.16 (m, 1H), 2.11-1.98 (m, 2H), 1.95-1.83 (m, 3H), 1.70-1.55 (m, 3H), 1.50-1.39 (m, 1H), 0.98 (d, 3H); LCMS: m/z 509.2 [M+H]⁺; >99% ee.

Diastereomer #2 (Example 96): ¹H NMR (400 MHz, DMSO-d₆) δ 14.47 (s, 1H), 9.07 (d, 1H), 8.95 (s, 1H), 8.61 (d, 1H), 8.41 (s, 1H), 8.12 (s, 1H), 8.03 (d, 1H), 7.79-7.67 (m, 1H), 4.74-4.58 (m, 1H), 4.54-4.39 (m, 1H), 4.31-4.10 (m, 1H), 3.57-3.47 (m, 2H), 3.11-3.02 (m, 1H), 2.91-2.78 (m, 1H), 2.72-2.56 (m, 1H), 2.29-2.17 (m, 1H), 2.14-1.98 (m, 2H), 1.96-1.81 (m, 3H), 1.74-1.54 (m, 3H), 1.50-1.39 (m, 1H), 0.96 (d, 3H); LCMS: m/z 509.2 [M+H]⁺; >99% ee.

### Preparation of Example 178 via General Scheme 1B

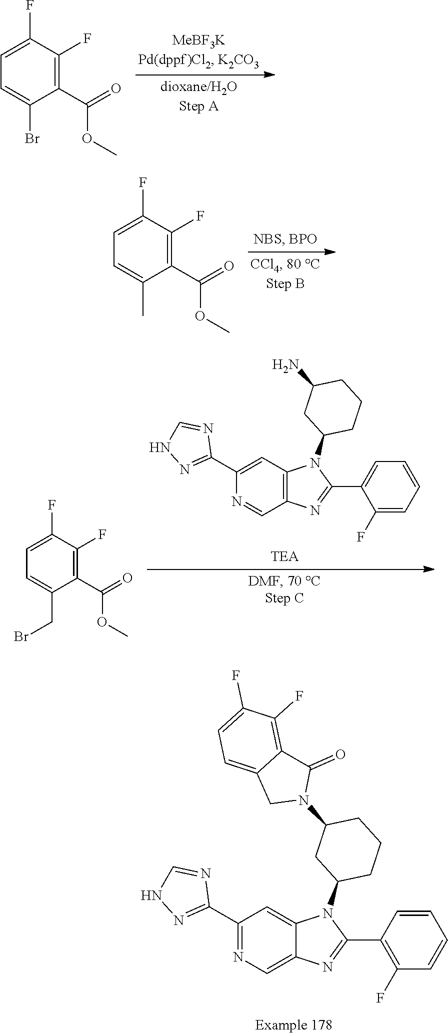

### Step A. Synthesis of methyl 2,3-difluoro-6-methylbenzoate

To the solution of methyl 6-bromo-2,3-difluorobenzoate (900 mg, 3.59 mmol), in 1,4-Dioxane/H₂O (10 mL) were added potassium trifluoro(methyl)borate (1.31 g, 10.8 mmol) and K₂CO₃ (1.49 g, 10.8 mmol) at rt, then the reaction mixture was stirred at 100° C. for 15 hrs. After completion, the reaction mixture was concentrated under reduced pressure. The residue was purified by flash column chromatography on silica gel to afford methyl 2,3-difluoro-6-methylbenzoate (320 mg, 48% yield). LCMS: m/z 187 [M+H]⁺.

### Step B. Synthesis of methyl 6-(bromomethyl)-2,3-difluorobenzoate

To a solution of methyl 2,3-difluoro-6-methylbenzoate (210 mg, 1.13 mmol) in CCl₄ (3 mL) were added BPO (301 mg, 1.24 mmol) and NBS (20 mg, 113 μmol), and then the reaction mixture was stirred at 80° C. for 15 hours under N₂. After completion, the reaction mixture was concentrated to dryness. The residue was purified by flash column chromatography on silica gel to afford methyl 6-(bromomethyl)-2,3-difluorobenzoate (105 mg, 35% yield). LCMS: m/z 265 [M+H]⁺.

### Step C. Synthesis of 6,7-difluoro-2-((1S,3R)-3-(2-(2-fluorophenyl)-6-(1H-1,2,4-triazol-3-yl)-1H-imidazo[4,5-c]pyridin-1-yl)cyclohexyl)isoindolin-1-one (Example 178)

To a solution of (1S,3R)-3-(2-(2-fluorophenyl)-6-(1H-1,2,4-triazol-3-yl)-1H-imidazo[4,5-c]pyridin-1-yl)cyclohexan-1-amine (100 mg, 265 μmol) in DMF (3 mL) were added TEA (161 mg, 1.59 mmol) and methyl 6-(bromomethyl)-2,3-difluorobenzoate (84 mg, 318 μmol) at rt. The reaction mixture was then stirred at 70° C. for 5 hrs. After completion, the reaction mixture was cooled to rt and purified by prep-HPLC to give Example 178 (18.2 mg, 13% yield). ¹H NMR (400 MHz, DMSO-d₆) δ 14.69 (s, 1H), 9.14 (s, 1H), 8.57 (s, 1H), 8.12 (s, 1H), 7.83-7.73 (m, 2H), 7.68-7.60 (m, 1H), 7.56-7.47 (m, 2H), 7.45-7.40 (m, 1H), 4.56-4.48 (m, 2H), 4.42-4.33 (m, 1H), 4.18-4.07 (m, 1H), 2.45-2.40 (m, 1H), 2.26-2.09 (m, 2H), 2.01-1.93 (m, 2H), 1.81-1.73 (m, 2H), 1.59-1.48 (m, 1H); LCMS: m/z 530.5 [M+H]⁺; >99% ee (based on enantiopurity of chiral intermediate).

### Preparation of Example 182 via General Scheme 1G

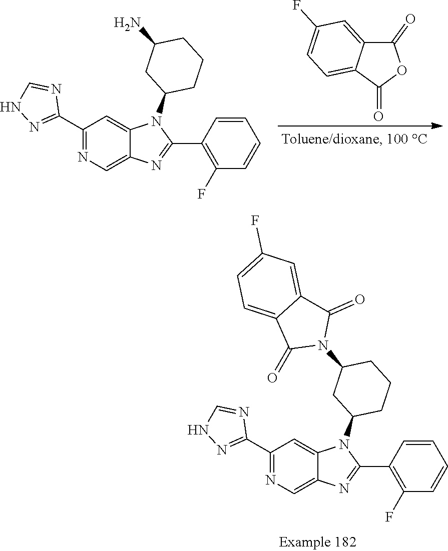 [SMILES: N[C@H]1CCC[C@@H](n2c(-c3ccccc3F)nc3cnc(-c4nc[nH]n4)cc32)C1 ; O=C1OC(=O)c2cc(F)ccc21 ; O=C1c2ccc(F)cc2C(=O)N1[C@H]1CCC[C@@H](n2c(-c3ccccc3F)nc3cnc(-c4nc[nH]n4)cc32)C1]

To the solution of (1S,3R)-3-(2-(2-fluorophenyl)-6-(1H-1,2,4-triazol-3-yl)-1H-imidazo[4,5-c]pyridin-1-yl)cyclohexan-1-amine (250 mg, 662 μmol) in toluene/dioxane (20 mL) was added 5-fluoroisobenzofuran-1,3-dione (110 mg, 662 μmol), and the reaction mixture was stirred at 100° C. for 15 hrs. After completion, the mixture was concentrated to dryness. The residue was purified by prep-HPLC to give 5-fluoro-2-((1S,3R)-3-(2-(2-fluorophenyl)-6-(1H-1,2,4-triazol-3-yl)-1H-imidazo[4,5-c]pyridin-1-yl)cyclohexyl)isoindoline-1,3-dione (Example 182) (150 mg, 43% yield). ¹H NMR (400 MHz, DMSO-d₆) δ 14.56 (s, 1H), 9.13 (s, 1H), 8.51 (s, 1H), 8.30-8.02 (m, 1H), 7.95-7.87 (m, 1H), 7.81-7.72 (m, 3H), 7.66-7.60 (m, 1H), 7.57-7.45 (m, 2H), 4.49-4.34 (m, 1H), 4.29-4.14 (m, 1H), 2.24-1.92 (m, 6H), 1.83-1.74 (m, 1H), 1.61-1.47 (m, 1H); LC-MS: m/z 526.5 [M+H]⁺; >99% ee (based on enantiopurity of chiral intermediate).

### Preparation of Example 202 via General Scheme 1D

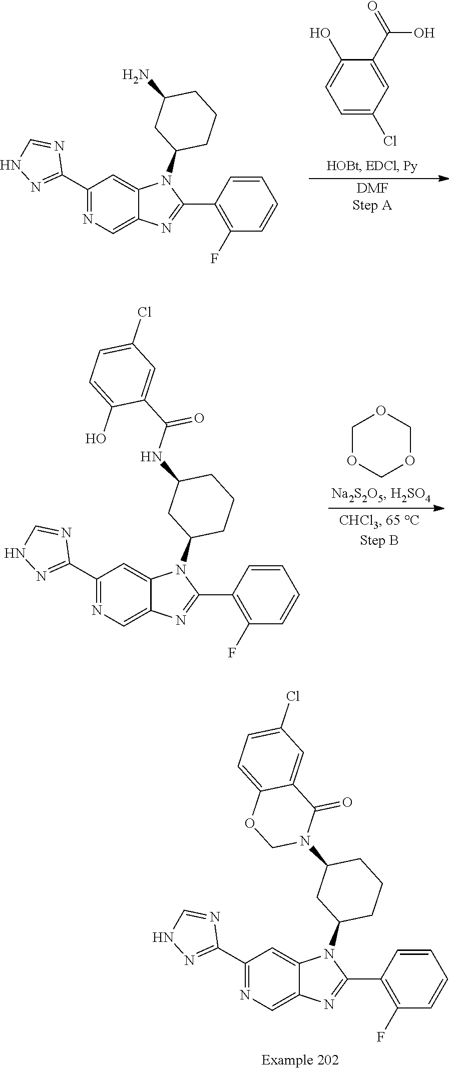 [SMILES: N[C@H]1CCC[C@@H](n2c(-c3ccccc3F)nc3cnc(-c4nc[nH]n4)cc32)C1 ; O=C(O)c1cc(Cl)ccc1O ; O=C(N[C@H]1CCC[C@@H](n2c(-c3ccccc3F)nc3cnc(-c4nc[nH]n4)cc32)C1)c1cc(Cl)ccc1O ; C1OCOCO1 ; O=C1c2cc(Cl)ccc2OCN1[C@H]1CCC[C@@H](n2c(-c3ccccc3F)nc3cnc(-c4nc[nH]n4)cc32)C1]

### Step A. Synthesis of 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-hydroxybenzamide

To a solution of (1S,3R)-3-(2-(2-fluorophenyl)-6-(1H-1,2,4-triazol-3-yl)-1H-imidazo[4,5-c]pyridin-1-yl)cyclohexan-1-amine (200 mg, 530 μmol) in DMF (5 mL) were added pyridine (168 mg, 2.12 mmol), 5-chloro-2-hydroxybenzoic acid (137 mg, 795 mol), HOBt (107 mg, 795 μmol) and EDCI (203 mg, 1.06 mmol). The reaction mixture was stirred at rt for 15 hrs. After completion, sat. aq. NH₄Cl solution (15 mL) was added, and the aqueous portion extracted with EtOAc (20 mL×3). The combined organic layers were washed with brine (20 mL), dried over Na₂SO₄, filtered, and concentrated to dryness. The residue was purified by flash column chromatography on silica gel to afford 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-hydroxybenzamide (150 mg, 53% yield). LC-MS: m/z 532 [M+H]⁺.

### Step B. Synthesis of 6-chloro-3-((1S,3R)-3-(2-(2-fluorophenyl)-6-(1H-1,2,4-triazol-3-yl)-1H-imidazo[4,5-c]pyridin-1-yl)cyclohexyl)-2,3-dihydro-4H-benzo[e][1,3]oxazin-4-one (Example 202)

To a solution of 5-chloro-N-((1S,3R)-3-(2-(2-fluorophenyl)-6-(1H-1,2,4-triazol-3-yl)-1H-imidazo[4,5-c]pyridin-1-yl)cyclohexyl)-2-hydroxybenzamide (80 mg, 0.15 mmol), 1,3,5-trioxane (270 mg, 3.0 mmol) and Na₂O₅S₂ (271 mg, 1.4 mmol) in CHCl₃ (15 mL) was added H₂SO₄ (5 drops). The reaction mixture was then stirred at 65° C. for 15 hrs and then allowed to cool. After completion, the reaction mixture was poured into ice-water (15 mL), and the aqueous portion extracted with EtOAc (20 mL×3). The combined organic layers were washed with brine (20 mL), dried over Na₂SO₄, filtered, and concentrated to dryness. The residue was purified by flash column chromatography on silica gel and then further purified by prep-HPLC to afford Example 202 (23 mg, 28% yield). ¹H NMR (400 MHz, DMSO-d₆) δ 14.57 (s, 1H), 9.12 (s, 1H), 8.66 (s, 1H), 8.21 (s, 1H), 7.81-7.68 (m, 3H), 7.59-7.44 (m, 3H), 7.10 (d, 1H), 5.51-5.35 (m, 2H), 4.37-4.17 (m, 2H), 2.60-2.52 (m, 1H), 2.36-2.12 (m, 1H), 2.05-1.84 (m, 4H), 1.72-1.59 (m, 1H), 1.55-1.38 (m, 1H); LC-MS: m/z 544.3 [M+H]⁺; >99% ee (based on enantiopurity of chiral intermediate).

### Preparation of Examples 203 and 207 according to General Schemes 1D

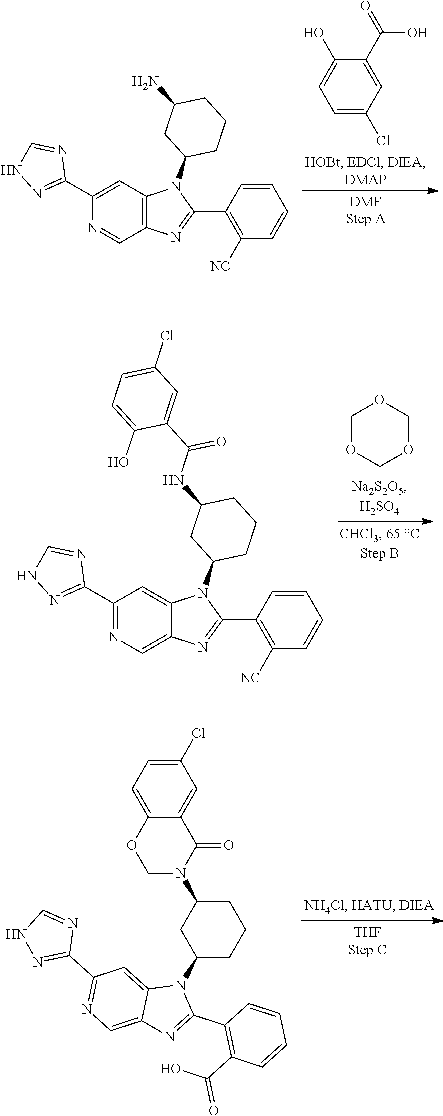 [SMILES: N#Cc1ccccc1-c1nc2cnc(-c3nc[nH]n3)cc2n1[C@@H]1CCC[C@H](N)C1 ; O=C(O)c1cc(Cl)ccc1O ; N#Cc1ccccc1-c1nc2cnc(-c3nc[nH]n3)cc2n1[C@@H]1CCC[C@H](NC(=O)c2cc(Cl)ccc2O)C1 ; C1OCOCO1 ; O=C(O)c1ccccc1-c1nc2cnc(-c3nc[nH]n3)cc2n1[C@@H]1CCC[C@H](N2COc3ccc(Cl)cc3C2=O)C1]

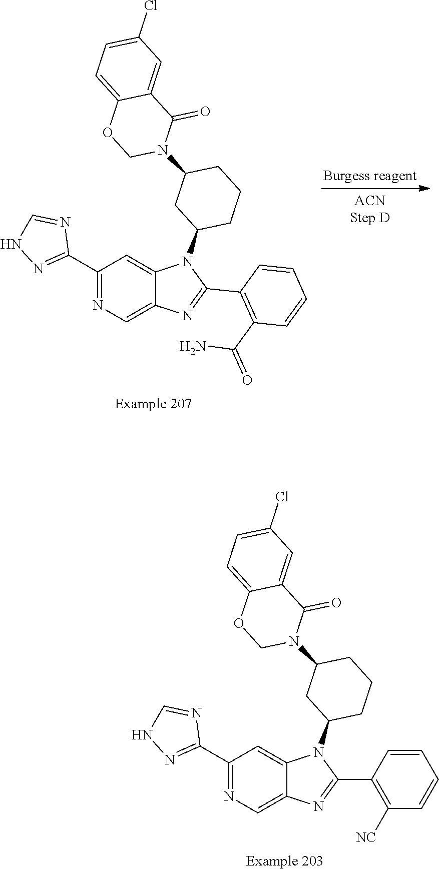 [SMILES: NC(=O)c1ccccc1-c1nc2cnc(-c3nc[nH]n3)cc2n1[C@@H]1CCC[C@H](N2COc3ccc(Cl)cc3C2=O)C1 ; N#Cc1ccccc1-c1nc2cnc(-c3nc[nH]n3)cc2n1[C@@H]1CCC[C@H](N2COc3ccc(Cl)cc3C2=O)C1]

### Step A. Synthesis of 5-chloro-N-((1S,3R)-3-(2-(2-cyanophenyl)-6-(1H-1,2,4-triazol-3-yl)-1H-imidazo[4,5-c]pyridin-1-yl)cyclohexyl)-2-hydroxybenzamide

To a solution of 2-(1-((1R,3S)-3-aminocyclohexyl)-6-(1H-1,2,4-triazol-3-yl)-1H-imidazo[4,5-c]pyridin-2-yl)benzonitrile (250 mg, 0.65 mmol) and 5-chloro-2-hydroxybenzoic acid (135 mg, 0.78 mmol) in DMF (1 mL) were added HOBt (149 mg, 0.98 mmol), EDCI (187 mg, 0.98 mmol), DIEA (252 mg, 1.95 mmol) and DMAP (238 mg, 1.95 mmol). The reaction mixture was stirred at rt for 16 hrs. After completion, the reaction mixture was purified by C18 column to afford 5-chloro-N-((1S,3R)-3-(2-(2-cyanophenyl)-6-(1H-1,2,4-triazol-3-yl)-1H-imidazo[4,5-c]pyridin-1-yl)cyclohexyl)-2-hydroxybenzamide (300 mg, 86% yield). LC-MS: m/z 539 [M+H]⁺.

### Step B. Synthesis of 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzoic acid

To a solution of 5-chloro-N-((1S,3R)-3-(2-(2-cyanophenyl)-6-(1H-1,2,4-triazol-3-yl)-1H-imidazo[4,5-c]pyridin-1-yl)cyclohexyl)-2-hydroxybenzamide (300 mg, 557 mol) and sulfuric acid (55 mg, 557 μmol) in CHCl₃ (3 mL) were added 1,3,5-trioxane (50 mg, 557 μmol) and Na₂S₂O₅ (529 mg, 2.78 mmol). The reaction mixture was stirred at 65° C. for 16 hrs and then allowed to cool. After completion, the reaction mixture was purified by C₁₈ column to afford 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzoic acid (100 mg, 32% yield). LC-MS: m/z 570 [M+H]⁺.

### Step C. Synthesis of 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzamide (Example 207)

To a solution of 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzoic acid (100 mg, 175 μmol) and DIEA (34 mg, 263 μmol) in THF (2 mL) were added HATU (100 mg, 263 μmol) and NH₄Cl (17 mg, 351 μmol). The reaction mixture was stirred at rt for 16 hrs. After completion, the reaction mixture was purified by C₁₈ column to afford Example 207 (5 mg, 5% yield). ¹H NMR (400 MHz, DMSO-d₆) δ 14.51 (s, 1H), 9.00 (s, 1H), 8.58 (s, 1H), 8.13 (s, 1H), 7.84 (d, 1H), 7.76-7.52 (m, 6H), 7.45-7.35 (m, 1H), 7.09 (d, 1H), 5.53-5.37 (m, 2H), 4.24-4.13 (m, 1H), 4.13-3.95 (m, 1H), 2.24-1.89 (m, 6H), 1.65-1.57 (m, 1H), 1.38-1.27 (m, 1H); LC-MS: m/z 569.1 [M+H]⁺; >99% ee (based on enantiopurity of chiral intermediate).

### Step D. Synthesis of 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzonitrile (Example 203)

To a solution of 2-(1-((1R,3S)-3-(6-chloro-4-oxo-2H-benzo[e][1,3]oxazin-3(4H)-yl)cyclohexyl)-6-(1H-1,2,4-triazol-3-yl)-1H-imidazo[4,5-c]pyridin-2-yl)benzamide (70 mg, 0.12 mmol) in ACN (2 mL) was added Burgess reagent (59 mg, 0.25 mmol). The reaction mixture was stirred at rt for 16 hrs. After completion, the reaction mixture was purified by C₁₈ column to afford Example 203 (1.2 mg, 2% yield). ¹H NMR (400 MHz, DMSO-d₆) δ 14.63 (s, 1H), 9.18 (s, 1H), 8.69 (s, 1H), 8.17 (d, 2H), 8.02-7.94 (m, 1H), 7.94-7.86 (m, 2H), 7.72 (d, 1H), 7.56 (dd, 1H), 7.11 (d, 1H), 5.52-5.38 (m, 2H), 4.36-4.23 (m, 2H), 2.42-2.16 (m, 2H), 2.07-2.00 (m, 2H), 1.95-1.85 (m, 2H), 1.71-1.61 (m, 1H), 1.59-1.40 (m, 1H); LC-MS: m/z 551.1 [M+H]⁺; >99% ee (based on enantiopurity of chiral intermediate).

### Preparation of Examples 208 and 209 according to General Scheme 1E (Method C)

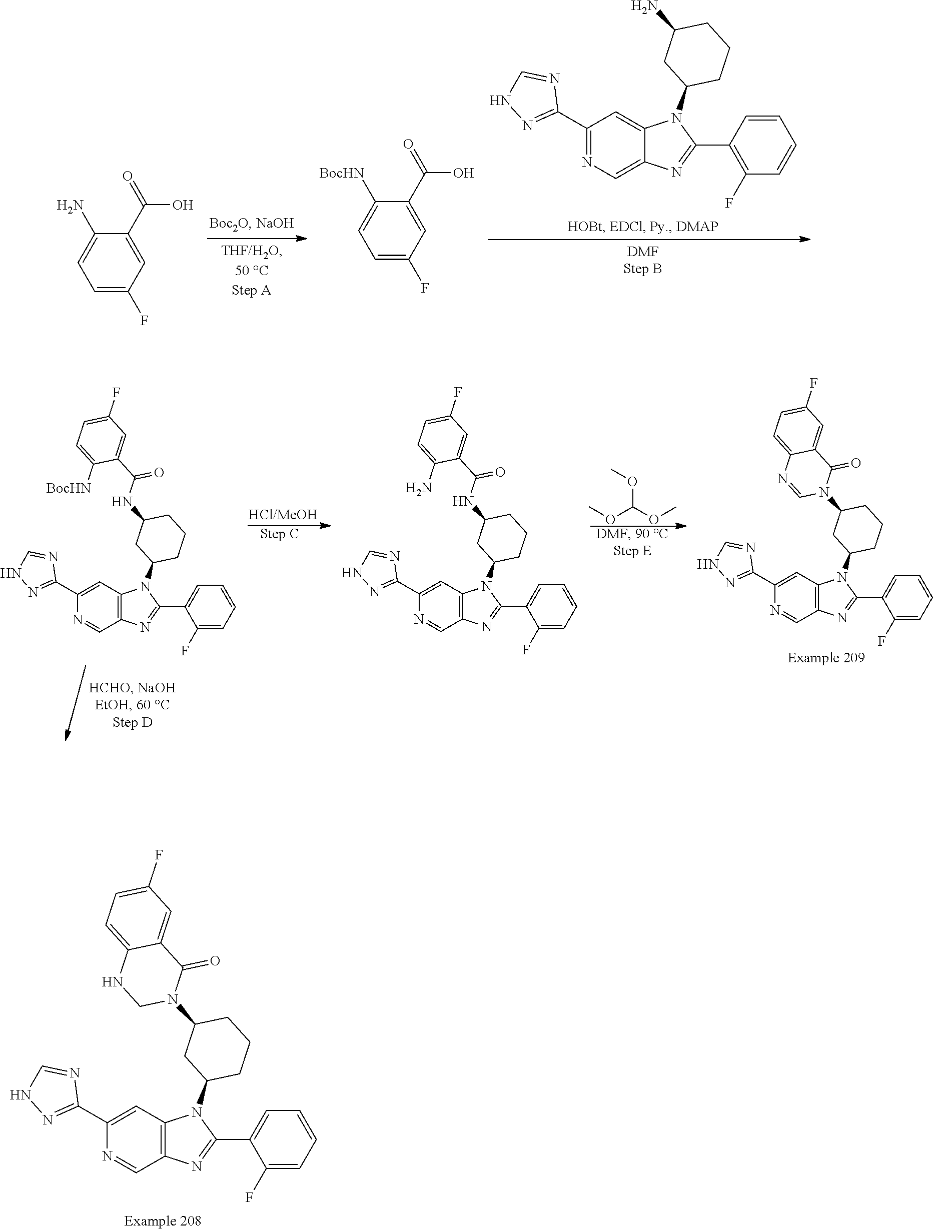

### Step A. Synthesis of 2-((tert-butoxycarbonyl)amino)-5-fluorobenzoic acid

To a solution of 2-amino-5-fluorobenzoic acid (2.5 g, 16 mmol) and NaOH (1.3 g, 32 mmol, 1 M in H₂O) in THF (25 mL) was added Boc₂O (4.2 g, 19 mmol) at 0° C. The reaction mixture was stirred at 50° C. under N₂ for 15 hrs and then allowed to cool. After completion, HCl (aq., 1 N) was added carefully until the pH was adjusted to pH=5. Water (20 mL) was added, and the aqueous portion extracted with EtOAc (50 mL×3). The combined organic layers were washed with brine (50 mL), dried over Na₂SO₄, filtered, and concentrated to dryness. The residue was purified by flash column chromatography on silica gel to afford 2-((tert-butoxycarbonyl)amino)-5-fluorobenzoic acid (2.43 g, 59% yield). LCMS: m/z 256 [M+H]⁺.

### Step B. Synthesis of tert-butyl (4-fluoro-2-(((1S,3R)-3-(2-(2-fluorophenyl)-6-(1H-1,2,4-triazol-3-yl)-1H-imidazo[4,5-c]pyridin-1-yl)cyclohexyl)carbamoyl)phenyl)carbamate

To a solution of (1S,3R)-3-(2-(2-fluorophenyl)-6-(1H-1,2,4-triazol-3-yl)-1H-imidazo[4,5-c]pyridin-1-yl)cyclohexan1-amine (200 mg, 530 μmol) and (2-((tert-butoxycarbonyl)amino)-5-fluorobenzoic acid (271 mg, 1.06 mmol) and pyridine (168 mg, 2.12 mmol) in DMF were added EDCI (203 mg, 1.06 mmol), HOBt (143 mg, 1.06 mmol) and DMAP (194 mg, 1.59 mmol). The reaction mixture was stirred at rt for 2 hrs. After completion, the reaction mixture was purified by C₁₈ column to afford tert-butyl (4-fluoro-2-(((1S,3R)-3-(2-(2-fluorophenyl)-6-(1H-1,2,4-triazol-3-yl)-1H-imidazo[4,5-c]pyridin-1-yl)cyclohexyl)carbamoyl)phenyl)carbamate (326 mg, 90% yield). LCMS (ESI): m/z 615 [M+H]⁺.

### Step C. Synthesis of 2-amino-5-fluoro-N-((1S,3R)-3-(2-(2-fluorophenyl)-6-(1H-1,2,4-triazol-3-yl)-1H-imidazo[4,5-c]pyridin-1-yl)cyclohexyl)benzamide

A solution of tert-butyl (4-fluoro-2-(((1S,3R)-3-(2-(2-fluorophenyl)-6-(1H-1,2,4-triazol-3-yl)-1H-imidazo[4,5-c]pyridin-1-yl)cyclohexyl)carbamoyl)phenyl)carbamate (328 mg, 534 μmol) in HCl/MeOH (6 mL, 4 M) was stirred at rt for 3 hrs. After completion, the reaction mixture was concentrated to dryness to afford 2-amino-5-fluoro-N-((1S,3R)-3-(2-(2-fluorophenyl)-6-(1H-1,2,4-triazol-3-yl)-1H-imidazo[4,5-c]pyridin-1-yl)cyclohexyl)benzamide (200 mg, quant.), which was used without further purification. LCMS: m/z 515 [M+H]⁺.

### Step D. Synthesis of 6-fluoro-3-((1S,3R)-3-(2-(2-fluorophenyl)-6-(1H-1,2,4-triazol-3-yl)-1H-imidazo[4,5-c]pyridin-1-yl)cyclohexyl)-2,3-dihydroquinazolin-4(1H)-one (Example 208)

To a solution of 2-amino-5-fluoro-N-((1S,3R)-3-(2-(2-fluorophenyl)-6-(1H-1,2,4-triazol-3-yl)-1H-imidazo[4,5-c]pyridin-1-yl)cyclohexyl)benzamide (70 mg, 0.14 mmol) and NaOH (6.0 mg, 0.15 mmol) in EtOH (2 mL) was added formaldehyde (4.1 mg, 0.14 mmol). The reaction mixture was stirred at 60° C. under N₂ for 1 hr and then allowed to cool. After completion, water (20 mL) was added, and the aqueous portion extracted with EtOAc (20 mL×3). The combined organic layers were washed with brine (10 mL), dried over Na₂SO₄, filtered, and concentrated to dryness. The residue was purified by prep-HPLC to afford Example 208 (14.1 mg, 20% yield). ¹H NMR (400 MHz, DMSO-d₆) δ 14.61 (s, 1H), 9.13 (s, 1H), 8.65 (s, 1H), 8.18 (s, 1H), 7.81-7.70 (m, 2H), 7.59-7.45 (m, 2H), 7.34 (dd, 1H), 7.18-7.12 (m, 1H), 6.80-6.73 (m, 1H), 6.70 (s, 1H), 4.64-4.50 (m, 2H), 4.42-4.33 (m, 1H), 4.32-4.19 (m, 1H), 2.49-2.42 (m, 1H), 2.33-2.17 (m, 1H), 2.00-1.91 (m, 3H), 1.89-1.80 (m, 1H), 1.66-1.57 (m, 1H), 1.52-1.39 (m, 1H); LCMS: m/z 527.5 [M+H]⁺; >99% ee (based on enantiopurity of chiral intermediate).

### Step E. Synthesis of 6-fluoro-3-((1S,3R)-3-(2-(2-fluorophenyl)-6-(1H-1,2,4-triazol-3-yl)-1H-imidazo[4,5-c]pyridin-1-yl)cyclohexyl)quinazolin-4(3H)-one (Example 209)

To a solution of 2-amino-5-fluoro-N-((1S,3R)-3-(2-(2-fluorophenyl)-6-(1H-1,2,4-triazol-3-yl)-1H-imidazo[4,5-c]pyridin-1-yl)cyclohexyl)benzamide (40 mg, 78 mol) in DMF (2 mL) was added trimethoxymethane (8 mg, 78 μmol). The reaction mixture was stirred at 90° C. under N₂ for 72 hrs and then allowed to cool. After completion, water (10 mL) was added, and the aqueous portion extracted with EtOAc (20 mL×3). The combined organic layers were washed with brine (10 mL), dried over Na₂SO₄, filtered, and concentrated to dryness. The residue was purified by prep-HPLC to afford Example 209 (16 mg, 39% yield). ¹H NMR (400 MHz, DMSO-d₆) δ 14.67 (s, 1H), 9.13 (s, 1H), 8.72 (s, 1H), 8.68 (s, 1H), 8.14 (s, 1H), 7.83-7.70 (m, 5H), 7.59-7.47 (m, 2H), 4.79-4.66 (m, 1H), 4.41-4.28 (m, 1H), 2.97-2.84 (m, 1H), 2.38-2.18 (m, 3H), 2.07-1.98 (m, 2H), 1.92-1.81 (m, 1H), 1.63-1.48 (m, 1H); LCMS: m/z 525.5 [M+H]⁺; >99% ee (based on enantiopurity of chiral intermediate).

### Preparation of Example 212 via General Scheme 1C

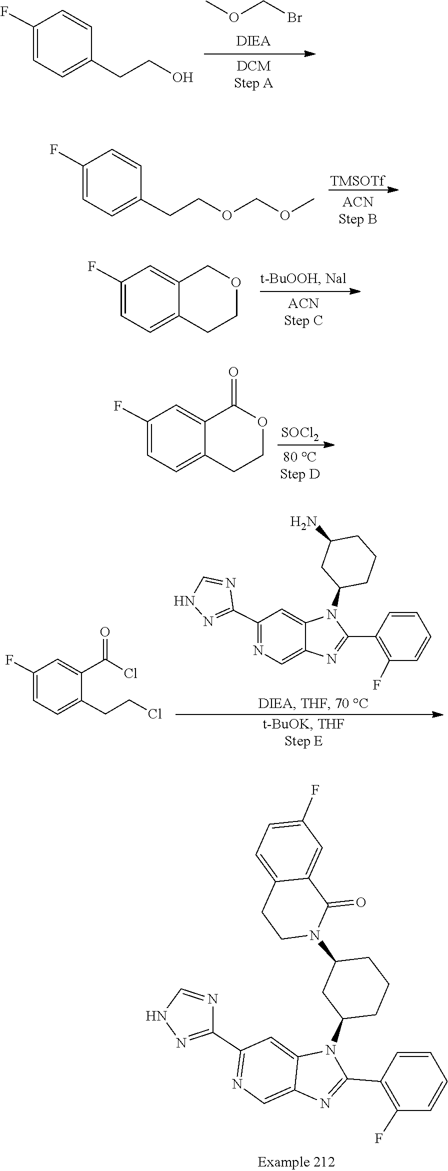

### Step A. Synthesis of 1-fluoro-4-(2-(methoxymethoxy)ethyl)benzene

To a solution of 2-(4-fluorophenyl)ethan-1-ol (1.5 g, 11 mmol) and bromo(methoxy)methane (2.0 g, 16 mmol) in DCM (20 mL) was added DIEA (4.1 g, 32 mmol), then the reaction mixture was stirred at rt for 16 hrs. After completion, water (20 mL) was added, and the aqueous portion extracted with EtOAc (30 mL×3). The combined organic layers were washed with brine (20 mL), dried over Na₂SO₄, filtered, and concentrated to dryness. The residue was purified by flash column chromatography on silica gel to afford 1-fluoro-4-(2-(methoxymethoxy)ethyl)benzene (1.8 g, 91% yield). LC-MS: m/z 185 [M+H]⁺.

### Step B. Synthesis of 7-fluoroisochromane

To a solution of 1-fluoro-4-(2-(methoxymethoxy)ethyl)benzene (2 g, 10.9 mmol) in ACN (20 mL) was added TMSOTf (3 g, 10.9 mmol), and the reaction mixture was stirred at rt for 16 hrs. After completion, water (20 mL) was added, and the aqueous portion extracted with EtOAc (30 mL×3). The combined organic layers were washed with brine (20 mL), dried over Na₂SO₄, filtered, and concentrated to dryness. The residue was purified by flash column chromatography on silica gel to afford 7-fluoroisochromane (1 g, 60% yield). LC-MS: m/z 153 [M+H]⁺.

### Step C. Synthesis of 7-fluoroisochroman-1-one

To a solution of 7-fluoroisochromane (1 g, 7 mmol) in ACN (20 mL) were added tert-Butyl hydroperoxide (3 g, 70 wt %, 2.1 mmol) and NaI (0.1 g, 0.7 mmol), and the reaction mixture was stirred at rt for 16 hrs. After completion, water (20 mL) was added, and the aqueous portion extracted with EtOAc (30 mL×3). The combined organic layers were washed with brine (20 mL), dried over Na₂SO₄, filtered, and concentrated to dryness. The residue was purified by flash column chromatography on silica gel to afford 7-fluoroisochroman-1-one (800 mg, 70% yield). LC-MS: m/z 167 [M+H]⁺.

### Step D. Synthesis of 2-(2-chloroethyl)-5-fluorobenzoyl chloride

A solution of 7-fluoroisochroman-1-one (100 mg, 602 μmol) in SOCl₂ (2 mL) was stirred at 80° C. for 16 hrs. After completion, the reaction mixture was concentrated to dryness to afford 2-(2-chloroethyl)-5-fluorobenzoyl chloride, which was used without further purification.

### Step E. Synthesis of 7-fluoro-2-((1S,3R)-3-(2-(2-fluorophenyl)-6-(1H-1,2,4-triazol-3-yl)-1H-imidazo[4,5-c]pyridin-1-yl)cyclohexyl)-3,4-dihydroisoquinolin-1(2H)-one (Example 212)

To a solution of 2-(2-chloroethyl)-5-fluorobenzoyl chloride (80 mg, 0.36 mmol) and (1S,3R)-3-(2-(2-fluorophenyl)-6-(1H-1,2,4-triazol-3-yl)-1H-imidazo[4,5-c]pyridin-1-yl)cyclohexan-1-amine (0.14 g, 0.36 mmol) in THF (5 mL) was added DIEA (0.14 g, 1.1 mmol). The reaction mixture was stirred at 70° C. for 16 hrs, and then was concentrated to dryness. The residue (120 mg, 161 μmol) was dissolved in THF (5 mL), and t-BuOK (54 mg, 482 μmol) was added. The reaction mixture was stirred at rt for 2 hrs. Water (20 mL) was added to the reaction mixture, and the aqueous portion extracted with EtOAc (30 mL×3). The combined organic layers were washed with brine (20 mL), dried over Na₂SO₄, filtered, and concentrated to dryness. The residue was purified by prep-HPLC to give (Example 212) (70 mg, 83% yield). ¹H NMR (400 MHz, DMSO-d₆) δ 14.45 (s, 1H), 9.13 (s, 1H), 8.60 (s, 1H), 8.20 (s, 1H), 7.80-7.70 (m, 2H), 7.57-7.45 (m, 3H), 7.37-7.26 (m, 2H), 4.51 (t, 1H), 4.31 (t, 1H), 3.60-3.51 (m, 2H), 2.89 (t, 2H), 2.48-2.41 (m, 1H), 2.29-2.15 (m, 1H), 2.03-1.90 (m, 3H), 1.88-1.73 (m, 1H), 1.68-1.57 (m, 1H), 1.57-1.40 (m, 1H); LC-MS: m/z 526.3 [M+H]⁺; >99% ee (based on enantiopurity of chiral intermediate).

### Preparation of Example 230 Via General Scheme 1A

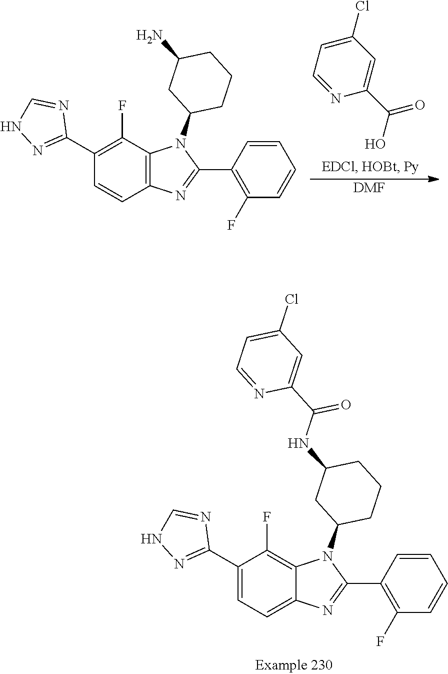

### Synthesis of 4-chloro-N-((1S,3R)-3-(7-fluoro-2-(2-fluorophenyl)-6-(1H-1,2,4-triazol-3-yl)-1H-benzo[d]imidazol-1-yl)cyclohexyl)picolinamide (Example 230)

To a solution of (1S,3R)-3-(7-fluoro-2-(2-fluorophenyl)-6-(1H-1,2,4-triazol-3-yl)-1H-benzo[d]imidazol-1-yl)cyclohexan-1-amine (400 mg, 426 μmol, 42% wt) and 4-chloropicolinic acid (87 mg, 554 μmol) in DMF (5 mL) were added pyridine (101 mg, 1.28 mmol), EDCI (122 mg, 639 μmol) and HOBt (98 mg, 639 μmol). The reaction mixture was stirred at rt for 2 hrs and purified by prep-HPLC to give Example 230 (55 mg, 23% yield). ¹H NMR (400 MHz, DMSO-d₆) δ 14.29 (s, 1H), 8.98 (s, 1H), 8.70 (s, 1H), 8.60 (s, 1H), 7.99 (s, 1H), 7.90 (s, 1H), 7.78-7.66 (m, 4H), 7.59-7.45 (m, 2H), 4.32-4.11 (m, 1H), 3.95-3.81 (m, 1H), 2.47-2.32 (m, 1H), 2.20-2.06 (m, 2H), 1.97-1.78 (m, 3H), 1.62-1.50 (m, 1H), 1.49-1.35 (m, 1H); LC-MS: m/z 534.2 [M+H]⁺; >99% ee (based on enantiopurity of chiral intermediate).

### Preparation of Examples 231 and 232 via General Scheme 1A

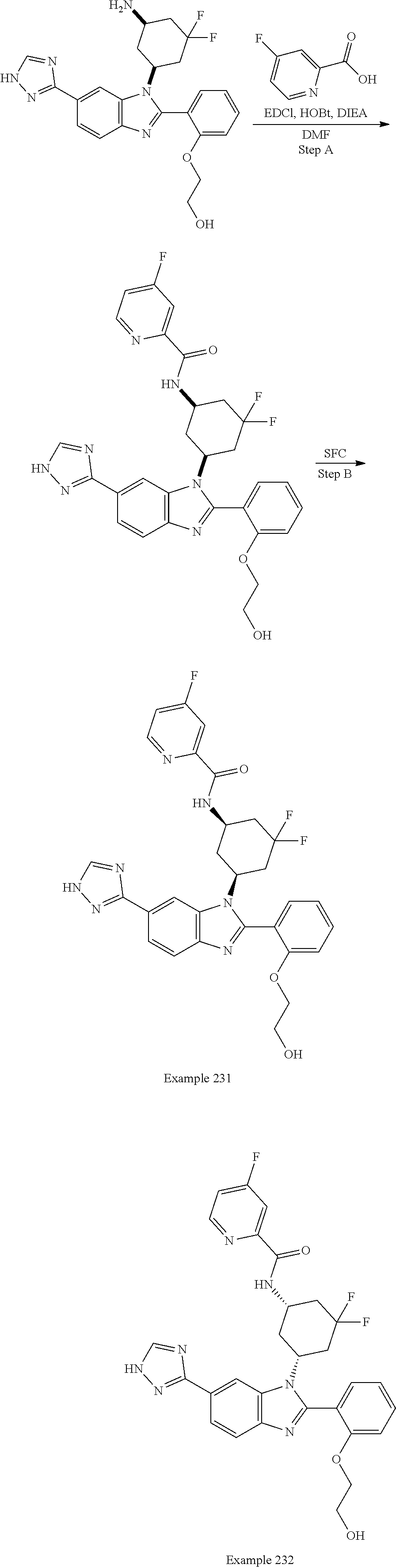

### Step A. Synthesis of N-(3,3-difluoro-5-(2-(2-(2-hydroxyethoxy)phenyl)-6-(1H-1,2,4-triazol-3-yl)-1H-benzo[d]imidazol-1-yl)cyclohexyl)-4-fluoropicolinamide (relative stereochemistry at centers 1 and 5 is cis)

To a solution of 2-(2-(1-(5-amino-3,3-difluorocyclohexyl)-6-(1H-1,2,4-triazol-3-yl)-1H-benzo[d]imidazol-2-yl)phenoxy)ethan-1-ol (186 mg, 409 μmol) in DMF (4 mL) were added 4-fluoropicolinic acid (57.7 mg, 409 μmol), DIEA (158 mg, 1.23 mmol), EDCI (118 mg, 614 μmol) and HOBt (83 mg, 614 μmol), and the reaction mixture was stirred at rt for 12 hrs. After completion, the reaction mixture was purified by prep-HPLC to afford N-(3,3-difluoro-5-(2-(2-(2-hydroxyethoxy)phenyl)-6-(1H-1,2,4-triazol-3-yl)-1H-benzo[d]imidazol-1-yl)cyclohexyl)-4-fluoropicolinamide (relative stereochemistry at centers 1 and 5 is cis) (210 mg, 89% yield). LC-MS: m/z 578.5 [M+H]⁺.

### Step B. Synthesis of Examples 231 and 232

N-(3,3-difluoro-5-(2-(2-(2-hydroxyethoxy)phenyl)-6-(1H-1,2,4-triazol-3-yl)-1H-benzo[d]imidazol-1-yl)cyclohexyl)-4-fluoropicolinamide (80 mg,) was further separated by chiral SFC to afford Examples 231 (15 mg, 19% yield, Rt=2.036 min) and Example 232 (15 mg, 19% yield, Rt=2.541 min). SFC method: (column: ChiralCel OX (250*21.2 mm, 5 μm), mobile phase: [0.1% NH₃H₂O MeOH]; %: 30%-30%,min).

Example 231, N-((1R,5S)-3,3-difluoro-5-(2-(2-(2-hydroxyethoxy)phenyl)-6-(1H-1,2,4-triazol-3-yl)-1H-benzo[d]imidazol-1-yl)cyclohexyl)-4-fluoropicolinamide: ¹H NMR (400 MHz, DMSO-d₆) δ 14.30 (s, 1H), 9.18 (s, 1H), 8.67 (s, 1H), 8.51-8.23 (m, 2H), 7.96 (d, 1H), 7.80 (d, 2H), 7.65-7.52 (m, 2H), 7.49 (d, 1H), 7.32 (d, 1H), 7.16 (t, 1H), 4.95-4.49 (m, 1H), 4.31-4.19 (m, 1H), 4.19-3.98 (m, 3H), 3.71-3.53 (m, 2H), 3.05-2.70 (m, 2H), 2.50-2.25 (m, 3H), 1.99-1.75 (m, 1H); LC-MS: m/z 578.5 [M+H]⁺; >99% ee.

Example 232, N-((1S,5R)-3,3-difluoro-5-(2-(2-(2-hydroxyethoxy)phenyl)-6-(1H-1,2,4-triazol-3-yl)-1H-benzo[d]imidazol-1-yl)cyclohexyl)-4-fluoropicolinamide: ¹H NMR (400 MHz, DMSO-d₆) δ 14.27 (s, 1H), 9.18 (s, 1H), 8.68 (s, 1H), 8.39 (s, 1H), 8.26 (s, 1H), 7.95 (d, 1H), 7.80 (d, 2H), 7.67-7.52 (m, 2H), 7.49 (d, 1H), 7.38-7.27 (m, 1H), 7.16 (t, 1H), 5.07-4.59 (m, 1H), 4.30-4.19 (m, 1H), 4.19-3.93 (m, 3H), 3.72-3.50 (m, 2H), 3.05-2.70 (m, 2H), 2.50-2.11 (m, 3H), 1.98-1.72 (m, 1H); LC-MS: m/z 578.5 [M+H]⁺; 92% ee.

### Preparation of Example 242 via General Schemes 1B and 8

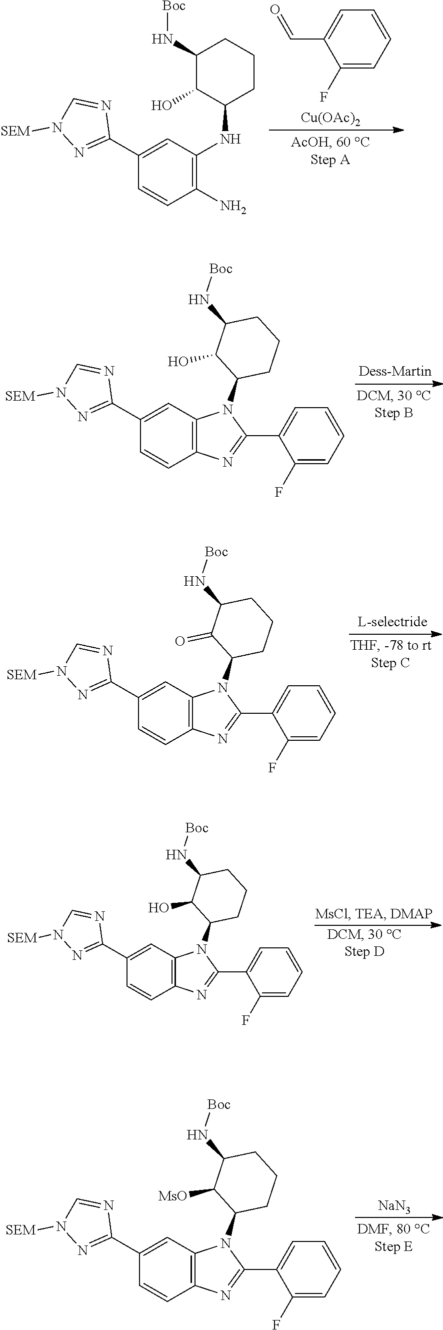

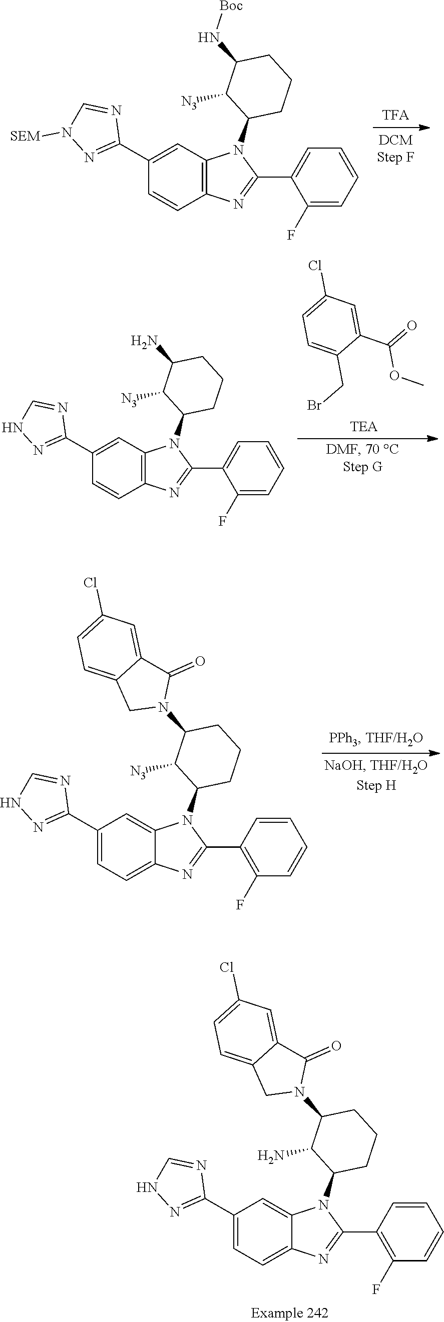

### Step A. Synthesis of tert-butyl ((1S,2R,3R)-3-(2-(2-fluorophenyl)-6-(1-((2-(trimethylsilyl)ethoxy)methyl)-1H-1,2,4-triazol-3-yl)-1H-benzol[d]imidazol-1-yl)-2-hydroxycyclohexyl)carbamate

To a mixture of tert-butyl ((1S,2S,3R)-3-((2-amino-5-(1-((2-(trimethylsilyl)ethoxy)methyl)-1H-1,2,4-triazol-3-yl)phenyl)amino)-2-hydroxycyclohexyl)carbamate (480 mg, 925 μmol) in AcOH (60 mL) was added 2-fluorobenzaldehyde (230 mg, 1.85 mmol) and then the reaction mixture was stirred at 60° C. for 3 hrs. Cupric acetate monohydrate (185 mg, 925 μmol) was added, and the reaction mixture was stirred at 60° C. for 24 hrs. After completion, the reaction mixture was cooled to rt and filtered. Water (15 mL) was added to the filtrate, and the aqueous portion extracted with EtOAc (15 mL). The combined organic layers were washed with brine (10 mL), dried over Na₂SO₄, filtered, and concentrated to dryness. The residue was purified by flash column chromatography on silica gel to afford tert-butyl ((1S,2R,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (360 mg, 63% yield). LC-MS: m/z 623 [M+H]⁺.

### Step B. Synthesis of tert-butyl ((1S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-oxocyclohexyl)carbamate

To a solution of tert-butyl ((1S,2R,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (120 mg, 93 μmol) in DCM (5 mL) was added dess-martinperiodinane (79 mg, 186 μmol) at 0° C. The reaction mixture was stirred at 30° C. for 16 hrs. After completion, the resulting mixture was concentrated under reduced pressure. The residue was purified by flash column chromatography on silica gel to afford tert-butyl ((1S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-oxocyclohexyl)carbamate (80 mg, 67% yield). LC-MS: m/z 621 [M+H]⁺.

### Step C. Synthesis of tert-butyl ((1S,2S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate

To a solution of tert-butyl ((1S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-oxocyclohexyl)carbamate (50 mg, 81 μmol) in THF (5 mL) was added lithium tri-sec-butylborohydride (L-selectride) (1M in THF) (0.2 mL, 0.16 mmol) at −78° C. under N₂ atmosphere. The reaction mixture was stirred at rt for 16 hrs. After completion, the resulting mixture was quenched by the addition of aq. sat. NH₄Cl solution (10 mL), and the aqueous portion extracted with EtOAc (10 mL×3). The combined organic layers were dried over Na₂SO₄, filtered, and concentrated to dryness. The residue was purified by flash column chromatography on silica gel to afford tert-butyl ((1S,2S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (20 mg, 40% yield). LC-MS: m/z 623 [M+H]⁺.

### Step D. Synthesis of (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl methanesulfonate

To a solution of tert-butyl ((1S,2S,3R)-3-(2-(2-fluorophenyl)-6-(1-((2-(trimethylsilyl)ethoxy)methyl)-1H-1,2,4-triazol-3-yl)-1H-benzo[d]imidazol-1-yl)-2-hydroxycyclohexyl)carbamate (270 mg, 434 μmol) in THF (10 mL) were added TEA (132 mg, 1.30 mmol), MsCl (99 mg, 867 μmol) and DMAP (159 mg, 1.30 mmol) at 30° C. Then the mixture was stirred at 30° C. for 3 hrs. After completion, the reaction mixture was quenched by the addition of sat. aq. NH₄Cl solution (10 mL), and the aqueous portion extracted with EtOAc (10 mL×3). The combined organic layers were dried over Na₂SO₄, filtered, and concentrated to dryness. The residue was purified by flash column chromatography on silica gel to afford (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl methanesulfonate (270 mg, 89% yield). LC-MS: m/z 701 [M+H]⁺.

### Step E. Synthesis of tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate

To a solution of (1S,2S,6R)-2-((tert-butoxycarbonyl)amino)-6-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl methanesulfonate (300 mg, 428 μmol) in DMF (5 mL) was added sodium azide (84 mg, 1.28 mmol). The reaction mixture was stirred at 80° C. for 16 hrs and then allowed to cool. After completion, the resulting mixture was purified by prep-HPLC to afford tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate (40 mg, 14% yield). LC-MS: m/z 648 [M+H]⁺.

### Step F. Synthesis of (1S,2R,3R)-2-azido-3-(2-(2-fluorophenyl)-6-(1H-1,2,4-triazol-3-yl)-1H-benzo[d]imidazol-1-yl)cyclohexan-1-amine

To a solution of tert-butyl ((1S,2R,3R)-2-azido-3-(2-(2-fluorophenyl)-6-(1-((2-(trimethylsilyl)ethoxy)methyl)-1H-1,2,4-triazol-3-yl)-1H-benzo[d]imidazol-1-yl)cyclohexyl)carbamate (35 mg, 54 μmol) in DCM (10 mL) was added TFA (1 mL), the reaction mixture was stirred at rt for 6 hrs. After completion, the reaction mixture was concentrated to dryness to afford (1S,2R,3R)-2-azido-3-(2-(2-fluorophenyl)-6-(1H-1,2,4-triazol-3-yl)-1H-benzo[d]imidazol-1-yl)cyclohexan-1-amine (22 mg, quant.), which was used without further purification. LC-MS: m/z 418 [M+H]⁺.

### Step G. Synthesis of 2-((1S,2S,3R)-2-azido-3-(2-(2-fluorophenyl)-6-(1H-1,2,4-triazol-3-yl)-1H-benzol[d]imidazol-1-yl)cyclohexyl)-6-chloroisoindolin-1-one

To a solution of (1S,2R,3R)-2-azido-3-(2-(2-fluorophenyl)-6-(1H-1,2,4-triazol-3-yl)-1H-benzo[d]imidazol-1-yl)cyclohexan-1-amine (25 mg, 60 μmol) and methyl 2-(bromomethyl)-5-chlorobenzoate (16 mg, 60 μmol) in DMF (2 mL) was added TEA (36 mg, 0.36 mmol). The reaction mixture was stirred at 70° C. for 6 hrs and then allowed cool. After completion, the reaction mixture was purified by prep-HPLC to give 2-((1S,2S,3R)-2-azido-3-(2-(2-fluorophenyl)-6-(1H-1,2,4-triazol-3-yl)-1H-benzo[d]imidazol-1-yl)cyclohexyl)-6-chloroisoindolin-1-one (15 mg, 44% yield). LC-MS: m/z 568 [M+H]⁺.

### Step H. Synthesis of 2-((1S,2S,3R)-2-amino-3-(2-(2-fluorophenyl)-6-(1H-1,2,4-triazol-3-yl)-1H-benzo[d]imidazol-1-yl)cyclohexyl)-6-chloroisoindolin-1-one (Example 242)

To a solution of 2-((1S,2S,3R)-2-azido-3-(2-(2-fluorophenyl)-6-(1H-1,2,4-triazol-3-yl)-1H-benzo[d]imidazol-1-yl)cyclohexyl)-6-chloroisoindolin-1-one (10 mg, 18 mol) in THF (2 mL) and water (1 mL) was added triphenylphosphane (9 mg, 35 μmol). The reaction mixture was stirred at rt for 16 hrs. After completion, water (5 mL) was added, and the aqueous portion extracted with EtOAc (10 mL×3). The combined organic layers were dried over Na₂SO₄, filtered, and concentrated to dryness to afford the intermediate 6-chloro-2-((1S,2S,3R)-3-(2-(2-fluorophenyl)-6-(1H-1,2,4-triazol-3-yl)-1H-benzo[d]imidazol-1-yl)-2-((triphenyl-15-phosphaneylidene)amino)cyclohexyl)isoindolin-1-one.

The intermediate (15 mg, 19 μmol) was dissolved in THF/H₂O (2 mL/1 mL) and 1M NaOH (1 mL) was added. The reaction mixture was stirred at rt for 72 hrs. After completion, the mixture was purified by prep-HPLC directly to give Example 242 (2 mg, 21% yield). ¹H NMR (400 MHz, CD₃OD) δ 8.58 (s, 1H), 8.44 (s, 1H), 8.05 (d, 1H), 7.84 (d, 1H), 7.78-7.68 (m, 3H), 7.59-7.53 (m, 2H), 7.49-7.38 (m, 2H), 4.62 (s, 2H), 4.12-3.90 (m, 3H), 2.76-2.61 (m, 1H), 2.34-2.04 (m, 3H), 1.96-1.87 (m, 1H), 1.75-1.56 (m, 1H); LC-MS: m/z 542.3 [M+H]⁺; >99% ee.

The compounds of Tables 1 and 3 were characterized using proton NMR and LCMS and the enantiomeric excess determined. See Table 5

TABLE 5

| Ex. # | ¹H NMR | LCMS | %ee |
|---|---|---|---|
| 1 | (400 MHz, DMSO-d₆) δ 14.63 (s, 1H), 9.14 (s, 1H), 8.45 | m/z 546.2 | >99%* |
| | (s, 1H), 8.27 (d, 1H), 8.20 (s, 1H), 7.75 (t, 2H), 7.59-7.46 | [M + H]⁺ | |
| | (m, 4H), 7.11 (d, 1H), 4.32-4.18 (m, 1H), 3.94-3.85 (m, | | |
| | 1H), 3.81 (s, 3H), 2.37-2.26 (m, 1H), 2.20-2.08 (m, 2H), | | |
| | 2.00-1.87 (m, 3H), 1.52-1.36 (m, 2H) | | |
| 2 | (400 MHz, DMSO-d₆) δ 14.66 (s, 1H), 9.14 (s, 1H), 8.49 | m/z 530.2 | >99%* |
| | (d, 1H), 8.44 (s, 1H), 8.22-7.99 (m, 1H), 7.79-7.71 (m, | [M + H]⁺ | |
| | 2H), 7.58-7.46 (m, 2H), 7.40-7.31 (m, 1H), 6.87 (d, 1H), | | |
| | 6.79 (t, 1H), 4.33-4.20 (m, 1H), 3.94-3.83 (m, 1H), 3.73 | | |
| | (s, 3H), 2.37-2.12 (m, 3H), 2.08-1.90 (m, 3H), 1.51-1.34 | | |
| | (m, 2H) | | |
| 3 | (400 MHz, CDCl₃) δ 9.13 (s, 1H), 8.46 (s, 1H), 8.10 (s, | m/z 516.3 | >99%* |
| | 1H), 7.71-7.62 (m, 2H), 7.58 (d, 1H), 7.43-7.33 (m, 5H), | [M + H]⁺ | |
| | 6.29-6.17 (m, 1H), 4.33-4.24 (m, 1H), 4.22-4.10 (m, 1H), | | |
| | 2.43-2.09 (m, 6H), 1.52-1.48 (m, 2H) | | |
| 4 | (400 MHz, DMSO-d₆) δ 14.65 (s, 1H), 9.14 (s, 1H), 8.68 | m/z 600.3 | >99%* |
| | (d, 1H), 8.45 (s, 1H), 8.11 (s, 1H), 7.74 (t, 2H), 7.65-7.45 | [M + H]⁺ | |
| | (m, 5H), 4.36-4.21 (m, 1H), 3.98-3.81 (m, 1H), 2.37-2.23 | | |
| | (m, 1H), 2.20-2.05 (m, 2H), 2.00-1.81 (m, 3H), 1.53-1.32 | | |
| | (m, 2H) | | |
| 5 | (400 MHz, DMSO-d₆) δ 14.43 (s, 1H), 9.14 (s, 1H), 8.49 | m/z 512.3 | >99%* |
| | (s, 1H), 8.25-8.12 (m, 2H), 7.80-7.71 (m, 2H), 7.57-7.47 | [M + H]⁺ | |
| | (m, 3H), 7.45-7.37 (m, 1H), 7.08 (d, 1H), 6.97 (t, 1H), | | |
| | 4.31-4.20 (m, 1H), 3.92-3.86 (m, 1H), 3.81 (s, 3H), 2.37- | | |
| | 2.26 (m, 1H)), 2.19-2.07 (m, 2H), 1.99-1.85 (m, 3H), | | |
| | 1.52-1.39 (m, 2H) | | |
| 6 | (400 MHz, DMSO-d₆) δ 14.61 (s, 1H), 9.14 (s, 1H), 8.48 | m/z 560.2 | >99%* |
| | (s, 1H), 8.28-8.13 (m, 2H), 7.79-7.68 (m, 2H), 7.54-7.40 | [M + H]⁺ | |
| | (m, 4H), 7.10 (d, 1H), 4.35-4.22 (m, 1H), 4.07 (q, 2H), | | |
| | 3.96-3.86 (m, 1H), 2.31-2.11 (m, 3H), 2.01-1.89 (m, 3H), | | |
| | 1.51-1.39 (m, 2H), 1.30 (t, 3H) | | |
| 7 | (400 MHz, DMSO-d₆) δ 14.66 (s, 1H), 9.13 (s, 1H), 9.07 | m/z 517.3 | >99%* |
| | (d, 1H), 8.60 (d, 1H), 8.53 (s, 1H), 8.13 (s, 1H), 7.99 (d, | [M + H]⁺ | |
| | 1H), 7.80-7.71 (m, 3H), 7.58-7.45 (m, 2H), 4.30-4.18 (m, | | |
| | 1H), 4.10-3.93 (m, 1H), 2.66-2.55 (m, 1H), 2.18-2.01 (m, | | |
| | 2H), 1.99-1.85 (m, 2H), 1.85-1.76 (m, 1H), 1.72-1.60 (m, | | |
| | 1H), 1.50-1.36 (m, 1H) | | |
| 8 | (400 MHz, DMSO-d₆) δ 14.63 (s, 1H), 9.14 (s, 1H), 8.48 | m/z 530.4 | >99%* |
| | (s, 1H), 8.32-7.99 (m, 2H), 7.83-7.67 (m, 2H), 7.57-7.45 | [M + H]⁺ | |
| | (m, 2H), 7.35-7.21 (m, 2H), 7.14-7.05 (m, 1H), 4.37-4.10 | | |
| | (m, 1H), 3.94-3.84 (m, 1H), 3.80 (s, 3H), 2.37-2.25 (m, | | |
| | 1H), 2.21-2.08 (m, 2H), 2.00-1.84 (m, 3H), 1.53-1.37 (m, | | |
| | 2H) | | |
| 9 | (400 MHz, DMSO-d₆) δ 14.63 (s, 1H), 9.14 (s, 1H), 8.48 | m/z 580.2 | >99%* |
| | (s, 1H), 8.34 (d, 1H), 8.22-8.01 (m, 1H), 7.77 (t, 4H), | [M + H]⁺ | |
| | 7.59-7.47 (m, 2H), 7.28 (d, 1H), 4.31-4.18 (m, 1H), 3.97- | | |
| | 3.91 (m, 1H), 3.89 (s, 3H), 2.35-2.28 (m, 1H), 2.22-2.07 | | |
| | (m, 2H), 2.03-1.91 (m, 3H), 1.55-1.44 (m, 2H) | | |
| 10 | (400 MHz, DMSO-d₆) δ 9.20 (s, 1H), 8.75 (d, 1H), 8.61- | m/z 517.4 | >99%* |
| | 8.48 (m, 2H), 8.36 (s, 1H), 8.00 (d, 1H), 7.76 (t, 2H), | [M + H]⁺ | |
| | 7.58-7.46 (m, 3H), 4.38-4.28 (m, 1H), 4.02-3.92 (m, 1H), | | |
| | 2.40-2.26 (m, 1H), 2.23-1.84 (m, 5H), 1.55-1.40 (m, 2H) | | |
| 11 | (400 MHz, DMSO-d₆) δ 14.66 (s, 1H), 9.13 (s, 1H), 8.84 | m/z 501.2 | >99%* |
| | (d, 1H), 8.50 (s, 1H), 8.44 (d, 1H), 8.10 (s, 1H), 7.88-7.79 | [M + H]⁺ | |
| | (m, 1H), 7.75 (t, 2H), 7.65-7.60 (m, 1H), 7.57-7.46 (m, | | |
| | 2H), 4.31-4.18 (m, 1H), 4.02-3.88 (m, 1H), 2.49-2.45 (m, | | |
| | 1H), 2.17-2.05 (m, 2H), 2.05-1.80 (m, 3H), 1.64-1.37 (m, | | |
| | 2H) | | |
| 12 | (400 MHz, DMSO-d₆) δ 9.16 (s, 1H), 8.79 (d, 1H), 8.47 | m/z 594.4 | >99%* |
| | (s, 1H), 8.18 (s, 1H), 7.92 (d, 1H), 7.80-7.70 (m, 3H), 7.62 | [M + H]⁺ | |
| | (d, 1H), 7.58-7.45 (m, 2H), 7.27-6.99 (m, 1H), 4.36-4.26 | | |
| | (m, 1H), 3.96-3.89 (m, 1H), 3.34 (s, 3H), 2.31-2.18 (m, | | |
| | 2H), 2.14-2.04 (m, 1H), 2.02-1.86 (m, 3H), 1.53-1.34 (m, | | |
| | 2H) | | |
| 13 | (400 MHz, DMSO-d₆) δ 14.47 (s, 1H), 9.13 (s, 1H), 9.01 | m/z 517.4 | >99%* |
| | (d, 1H), 8.66 (d, 1H), 8.53 (s, 1H), 8.21 (s, 1H), 8.09 (dd, | [M + H]⁺ | |
| | 1H), 8.00 (d, 1H), 7.80-7.68 (m, 2H), 7.60-7.45 (m, 2H), | | |
| | 4.32-4.16 (m, 1H), 4.08-3.91 (m, 1H), 2.72-2.56 (m, 1H), | | |
| | 2.15-2.02 (m, 2H), 1.99-1.86 (m, 2H), 1.83-1.76 (m, 1H), | | |
| | 1.73-1.61 (m, 1H), 1.50-1.41 (m, 1H) | | |
| 14 | (400 MHz, DMSO-d₆) δ 14.53 (s, 1H), 9.13 (s, 1H), 9.06 | m/z 501.4 | >99%* |
| | (d, 1H), 8.66 (dd, 1H), 8.53 (s, 1H), 8.20 (s, 1H), 7.80- | [M + H]⁺ | |
| | 7.71 (m, 3H), 7.57-7.45 (m, 3H), 4.33-4.19 (m, 1H), 4.06- | | |
| | 3.91 (m, 1H), 2.68-2.58 (m, 1H), 2.18-2.01 (m, 2H), 1.99- | | |
| | 1.84 (m, 2H), 1.82-1.74 (m, 1H), 1.72-1.58 (m, 1H), 1.51- | | |
| | 1.36 (m, 1H) | | |
| 15 | Data Provided Above | | |
| 16 | (400 MHz, DMSO-d₆) δ 14.62 (s, 1H), 9.17 (s, 1H), 9.05 | m/z 502.2 | >99%* |
| | (d, 1H), 8.73-8.64 (m, 2H), 8.57 (s, 1H), 8.21 (s, 1H), 8.08 | [M + H]⁺ | |
| | (t, 1H), 7.83-7.76 (m, 2H), 7.57-7.50 (m, 1H), 4.83-4.67 | | |
| | (m, 1H), 4.10-3.95 (m, 1H), 2.69-2.58 (m, 1H), 2.19-1.99 | | |
| | (m, 3H), 1.93-1.78 (m, 2H), 1.72-1.60 (m, 1H), 1.54-1.40 | | |
| | (m, 1H) | | |
| 17 | (400 MHz, DMSO-d₆) δ 14.68 (s, 1H), 9.56 (d, 1H), 9.51 | m/z 518.2 | >99%* |
| | (d, 1H), 9.13 (s, 1H), 8.52 (s, 1H), 8.28 (d, 1H), 8.11 (s, | [M + H]⁺ | |
| | 1H), 7.79-7.71 (m, 2H), 7.58-7.46 (m, 2H), 4.35-4.18 (m, | | |
| | 1H), 4.18-3.99 (m, 1H), 2.74-2.60 (m, 1H), 2.19-2.03 (m, | | |
| | 2H), 2.03-1.79 (m, 3H), 1.76-1.64 (m, 1H), 1.56-1.38 (m, | | |
| | 1H) | | |
| 18 | (400 MHz, DMSO-d₆) δ 9.16 (s, 1H), 8.52 (s, 1H), 8.27 (s, | m/z 574.4 | >99%* |
| | 1H), 8.20 (d, 1H), 7.75 (t, 2H), 7.58-7.46 (m, 3H), 7.43 | [M + H]⁺ | |
| | (dd, 1H), 7.14 (d, 1H), 4.70-4.59 (m, 1H), 4.40-4.27 (m, | | |
| | 1H), 3.98-3.85 (m, 1H), 2.26-2.16 (m, 2H), 2.16-2.04 (m, | | |
| | 1H), 2.04-1.86 (m, 3H), 1.54-1.36 (m, 2H), 1.30-1.21 (m, | | |
| | 6H) | | |
| 19 | (400 MHz, DMSO-d₆) δ 14.66 (s, 1H), 9.13 (s, 1H), 8.92 | m/z 501.5 | >99%* |
| | (d, 1H), 8.60 (d, 1H), 8.53 (s, 1H), 8.21-8.04 (m, 2H), | [M + H]⁺ | |
| | 7.91-7.82 (m, 1H), 7.80-7.70 (m, 2H), 7.58-7.44 (m, 2H), | | |
| | 4.32-4.16 (m, 1H), 4.09-3.91 (m, 1H), 2.68-2.55 (m, 1H), | | |
| | 2.21-2.02 (m, 2H), 1.97-1.85 (m, 2H), 1.83-1.75 (m, 1H), | | |
| | 1.73-1.58 (m, 1H), 1.54-1.37 (m, 1H) | | |
| 20 | (400 MHz, DMSO-d₆) δ 14.70 (s, 1H), 9.07 (d, 1H), 9.05 | m/z 495.2 | >99%* |
| | (s, 1H), 8.61 (d, 1H), 8.45 (s, 1H), 8.19 (s, 1H), 8.03 (d, | [M + H]⁺ | |
| | 1H), 7.76 (dd, 1H), 4.86 (s, 2H), 4.75-4.59 (m, 1H), 4.26- | | |
| | 4.06 (m, 1H), 3.83-3.70 (m, 1H), 2.69-2.54 (m, 1H), 2.16- | | |
| | 2.03 (m, 2H), 2.02-1.92 (m, 2H), 1.92-1.82 (m, 1H), 1.80- | | |
| | 1.65 (m, 1H), 1.64-1.50 (m, 1H), 1.18 (t, 6H) | | |
| 21 | Data provided above | | |
| 22 | (400 MHz, DMSO-d₆) δ 14.64 (s, 1H), 9.14 (s, 1H), 8.47 | m/z 547.2 | >99%* |
| | (s, 1H), 8.40 (d, 1H), 8.31 (d, 1H), 8.21-8.02 (m, 1H), 7.96 | [M + H]⁺ | |
| | (d, 1H), 7.75 (t, 2H), 7.58-7.46 (m, 2H), 4.33-4.19 (m, | | |
| | 1H), 3.96-3.85 (m, 4H), 2.36-2.23 (m, 1H), 2.23-2.07 (m, | | |
| | 2H), 2.00-1.87 (m, 3H), 1.52-1.37 (m, 2H) | | |
| 23 | (400 MHz, DMSO-d₆) δ 14.59 (s, 1H), 9.13 (s, 1H), 8.47 | m/z 561.2 | >99%* |
| | (s, 1H), 8.32 (d, 1H), 8.28 (d, 1H), 8.14 (s, 1H), 7.94 (d, | [M + H]⁺ | |
| | 1H), 7.74 (t, 2H), 7.58-7.45 (m, 2H), 4.39-4.22 (m, 3H), | | |
| | 3.96-3.84 (m, 1H), 2.33-2.09 (m, 3H), 2.02-1.85 (m, 3H), | | |
| | 1.54-1.39 (m, 2H), 1.29 (t, 3H) | | |
| 24 | (400 MHz, CD₃OD) δ 8.97 (s, 1H), 8.56 (d, 2H), 8.27 (s, | m/z 494.4 | >99%* |
| | 1H), 8.09 (d, 1H), 7.61 (dd, 1H), 4.99-4.92 (m, 1H), 4.26- | [M + H]⁺ | |
| | 4.15 (m, 1H), 4.13-3.99 (m, 2H), 2.69 (q, 2H), 2.56-2.44 | | |
| | (m, 1H), 2.37 (s, 3H), 2.35-2.27 (m, 2H), 2.16-2.03 (m, | | |
| | 3H), 1.76-1.62 (m, 2H), 1.22 (t, 3H) | | |
| 25 | (400 MHz, DMSO-d₆) δ 14.64 (s, 1H), 9.15 (s, 1H), 9.10 | m/z 500.5 | >99%* |
| | (d, 1H), 8.85 (d, 1H), 8.61 (d, 1H), 8.54 (s, 1H), 8.25 (d, | [M + H]⁺ | |
| | 1H), 8.17-8.05 (m, 2H), 8.00 (d, 1H), 7.75 (dd, 1H), 7.68- | | |
| | 7.61 (m, 1H), 5.74-5.51 (m, 1H), 4.15-3.95 (m, 1H), 2.77- | | |
| | 2.61 (m, 1H), 2.24-2.11 (m, 2H), 2.09-1.92 (m, 2H), 1.91- | | |
| | 1.81 (m, 1H), 1.78-1.62 (m, 1H), 1.57-1.43 (m, 1H) | | |
| 26 | (400 MHz, DMSO-d₆) δ 14.59 (s, 1H), 9.15 (s, 1H), 8.85 | m/z 543.5 | >99%* |
| | (d, 1H), 8.48 (s, 1H), 8.33-8.25 (m, 2H), 8.20-8.07 (m, | [M + H]⁺ | |
| | 2H), 7.71-7.60 (m, 1H), 7.51 (d, 1H), 7.44 (dd, 1H), 7.13- | | |
| | 7.05 (m, 1H), 5.70-5.58 (m, 1H), 4.07 (q, 2H), 4.03-3.93 | | |
| | (m, 1H), 2.41-2.29 (m, 2H), 2.24-2.05 (m, 2H), 2.01-1.93 | | |
| | (m, 2H), 1.60-1.44 (m, 2H), 1.31 (t, 3H) | | |
| 27 | (400 MHz, DMSO-d₆) δ 14.71 (s, 1H), 9.17 (s, 1H), 8.71 | m/z 561.4 | >99%* |
| | (d, 1H), 8.51 (s, 1H), 8.24 (d, 1H), 8.13-8.06 (m, 1H), | [M + H]⁺ | |
| | 7.86-7.77 (m, 1H), 7.51 (d, 1H), 7.44 (dd, 2H), 7.10 (d, | | |
| | 1H), 4.77 (s, 1H), 4.11-4.05 (m, 2H), 4.02-3.90 (m, 1H), | | |
| | 2.33-2.20 (m, 2H), 2.15-1.99 (m, 2H), 1.95-1.85 (m, 2H), | | |
| | 1.56-1.39 (m, 2H), 1.30 (t, 3H) | | |
| 28 | (400 MHz, DMSO-d₆) δ 8.84 (d, 1H), 8.63 (d, 1H), 8.54 | m/z 519.2 | >99%* |
| | (s, 1H), 8.01 (d, 1H), 7.77 (dd, 1H), 7.73-7.61 (m, 2H), | [M + H]⁺ | |
| | 7.52-7.39 (m, 2H), 6.51 (s, 1H), 4.11-3.85 (m, 2H), 3.61- | | |
| | 3.46 (m, 4H), 2.64-2.53 (m, 1H), 2.17-2.06 (m, 1H), 2.06- | | |
| | 1.97 (m, 4H), 1.95-1.74 (m, 4H), 1.68-1.52 (m, 1H), 1.45- | | |
| | 1.32 (m, 1H) | | |
| 29 | (400 MHz, DMSO-d₆) δ 14.66 (s, 1H), 9.14 (s, 1H), 8.47 | m/z 572.2 | >99%* |
| | (s, 1H), 8.10 (d, 2H), 7.80-7.71 (m, 2H), 7.58-7.48 (m, | [M + H]⁺ | |
| | 2H), 7.47 (d, 1H), 7.43 (d, 1H), 7.37 (d, 1H), 4.33-4.19 | | |
| | (m, 1H), 3.95-3.80 (m, 2H), 2.37-2.08 (m, 3H), 2.00-1.81 | | |
| | (m, 3H), 1.52-1.37 (m, 2H), 0.79-0.70 (m, 2H), 0.70-0.62 | | |
| | (m, 2H) | | |
| 30 | (400 MHz, DMSO-d₆) δ 14.61 (s, 1H), 9.11 (s, 1H), 9.06 | m/z 549.4 | >99%* |
| | (d, 1H), 8.66 (dd, 1H), 8.52 (s, 1H), 8.20 (s, 1H), 7.84- | [M + H]⁺ | |
| | 7.72 (m, 2H), 7.69 (d, 1H), 7.57-7.48 (m, 3H), 7.47-7.19 | | |
| | (m, 1H), 4.19-4.08 (m, 1H), 4.03-3.87 (m, 1H), 2.72-2.57 | | |
| | (m, 1H), 2.24-1.85 (m, 4H), 1.83-1.77 (m, 1H), 1.72-1.60 | | |
| | (m, 1H), 1.49-1.32 (m, 1H) | | |
| 31 | (400 MHz, DMSO-d₆) δ 14.68 (s, 1H), 9.14 (s, 1H), 8.56 | m/z 556.4 | >99%* |
| | (d, 1H), 8.48 (s, 1H), 8.20-8.05 (m, 2H), 7.87 (d, 1H), | [M + H]⁺ | |
| | 7.80-7.71 (m, 2H), 7.57-7.46 (m, 3H), 7.02 (d, 1H), 4.37- | | |
| | 4.20 (m, 1H), 4.06-3.92 (m, 1H), 2.46-2.33 (m, 1H), 2.24- | | |
| | 2.08 (m, 2H), 2.03-1.91 (m, 3H), 1.60-1.42 (m, 2H) | | |
| 32 | (400 MHz, DMSO-d₆) δ 14.68 (s, 1H), 9.17 (s, 1H), 8.56- | m/z 559.2 | >99%* |
| | 8.47 (m, 2H), 8.24 (s, 1H), 7.85-7.74 (m, 2H), 7.67-7.62 | [M + H]⁺ | |
| | (m, 1H), 7.62-7.48 (m, 3H), 7.29 (dd, 1H), 6.67 (d, 1H), | | |
| | 4.32-4.20 (m, 1H), 3.97-3.84 (m, 1H), 3.16-3.08 (m, 2H), | | |
| | 2.47-2.35 (m, 1H), 2.22-2.09 (m, 2H), 2.05-1.84 (m, 3H), | | |
| | 1.54-1.39 (m, 2H), 1.18 (t, 3H) | | |
| 33 | (400 MHz, DMSO-d₆) δ 14.68 (s, 1H), 9.14 (s, 1H), 8.46 | m/z 537.2 | >99%* |
| | (s, 1H), 8.34 (d, 1H), 8.11 (s, 1H), 7.89 (dd, 1H), 7.84 (d, | [M + H]⁺ | |
| | 1H), 7.79-7.71 (m, 2H), 7.60-7.46 (m, 2H), 7.26 (d, 1H), | | |
| | 4.33-4.17 (m, 1H), 3.95-3.88 (m, 1H), 3.87 (s, 3H), 2.36- | | |
| | 2.26 (m, 1H), 2.21-2.07 (m, 2H), 2.02-1.85 (m, 3H), 1.52- | | |
| | 1.37 (m, 2H) | | |
| 34 | (400 MHz, DMSO-d₆) & 14.67 (s, 1H), 9.14 (s, 1H), 8.54- | m/z 576.4 | >99%* |
| | 8.45 (m, 2H), 8.35-8.20 (m, 1H), 7.80-7.71 (m, 2H), 7.68 | [M + H]⁺ | |
| | (d, 1H), 7.58-7.45 (m, 3H), 7.18 (d, 1H), 5.53-5.37 (m, | | |
| | 1H), 4.35-4.23 (m, 1H), 4.22-4.09 (m, 2H), 4.03-3.90 (m, | | |
| | 1H), 3.85-3.75 (m, 2H), 2.39-2.17 (m, 2H), 2.16-2.01 (m, | | |
| | 1H), 2.00-1.83 (m, 3H), 1.53-1.37 (m, 2H) | | |
| 35 | (400 MHz, DMSO-d₆) δ 14.59 (s, 1H), 9.14 (s, 1H), 8.47 | m/z 629.6 | >99%* |
| | (s, 1H), 8.36 (d, 1H), 8.24-8.16 (m, 1H), 7.80-7.70 (m, | [M + H]⁺ | |
| | 2H), 7.61 (d, 1H), 7.59-7.45 (m, 3H), 7.14 (d, 1H), 4.38- | | |
| | 4.23 (m, 1H), 4.12 (t, 2H), 3.97-3.86 (m, 1H), 2.80-2.66 | | |
| | (m, 2H), 2.38-2.31 (m, 4H), 2.23-2.08 (m, 3H), 2.07-1.97 | | |
| | (m, 1H), 1.92 (d, 2H), 1.53-1.39 (m, 6H) | | |
| 36 | (400 MHz, DMSO-d₆) δ 14.64 (s, 1H), 9.18 (s, 1H), 8.53- | m/z 581.3 | >99%* |
| | 8.48 (m, 1H), 8.27-8.13 (m, 3H), 8.01-7.95 (m, 1H), 7.95- | [M + H]⁺ | |
| | 7.85 (m, 2H), 7.51 (d, 1H), 7.43 (dd, 1H), 7.14 (d, 1H), | | |
| | 4.69-4.56 (m, 1H), 4.40-4.31 (m, 1H), 3.95-3.83 (m, 1H), | | |
| | 2.29-2.19 (m, 2H), 2.11-2.02 (m, 2H), 1.94-1.84 (m, 2H), | | |
| | 1.46-1.34 (m, 2H), 1.24 (d, 6H) | | |
| 37 | (400 MHz, DMSO-d₆) δ 14.65 (s, 1H), 9.52 (d, 1H), 9.14 | m/z 573.5 | >99%* |
| | (s, 1H), 8.49 (s, 1H), 8.25 (s, 1H), 7.75 (t, 2H), 7.58-7.44 | [M + H]⁺ | |
| | (m, 4H), 7.36 (d, 1H), 4.39-4.25 (m, 1H), 3.95-3.84 (m, | | |
| | 1H), 3.43 (s, 2H), 2.29-2.18 (m, 2H), 2.11 (s, 6H), 2.01- | | |
| | 1.87 (m, 3H), 1.55-1.29 (m, 3H) | | |
| 38 | (400 MHz, DMSO-d₆) δ 14.64 (s, 1H), 9.07 (d, 1H), 8.97 | m/z 480.4 | >99%* |
| | (s, 1H), 8.61 (d, 1H), 8.40 (s, 1H), 8.24 (s, 1H), 8.16 (s, | [M + H]⁺ | |
| | 1H), 8.02 (d, 1H), 7.75 (dd, 1H), 4.92-4.74 (m, 1H), 4.23- | | |
| | 4.07 (m, 3H), 2.67-2.55 (m, 3H), 2.16-1.83 (m, 5H), 1.74- | | |
| | 1.53 (m, 2H), 1.05 (t, 3H) | | |
| 39 | (400 MHz, DMSO-d₆) δ 14.63 (s, 1H), 9.14 (s, 1H), 8.71 | m/z 604.6 | >99%* |
| | (d, 1H), 8.47 (s, 1H), 8.21 (s, 1H), 7.80-7.69 (m, 3H), | [M + H]⁺ | |
| | 7.57-7.45 (m, 3H), 7.14 (d, 1H), 5.02 (s, 1H), 4.35-4.23 | | |
| | (m, 1H), 4.07-3.94 (m, 1H), 3.88 (s, 2H), 2.43-2.29 (m, | | |
| | 1H), 2.25-2.03 (m, 2H), 2.02-1.85 (m, 3H), 1.54-1.37 (m, | | |
| | 2H), 1.23 (s, 3H), 1.13 (s, 3H) | | |
| 40 | (400 MHz, DMSO-d₆) δ 14.56 (s, 1H), 9.14 (s, 1H), 8.56 | m/z 546.0 | >99%* |
| | (d, 1H), 8.46 (s, 1H), 8.18 (s, 1H), 7.75 (t, 2H), 7.59-7.46 | [M + H]⁺ | |
| | (m, 4H), 7.39 (d, 1H), 5.32-5.16 (m, 1H), 4.53 (s, 2H), | | |
| | 4.37-4.20 (m, 1H), 3.99-3.83 (m, 1H), 2.38-2.24 (m, 1H), | | |
| | 2.22-2.05 (m, 2H), 2.02-1.88 (m, 3H), 1.51-1.35 (m, 2H) | | |
| 41 | (400 MHz, DMSO-d₆) δ 14.63 (s, 1H), 9.15 (s, 1H), 8.53- | m/z 531.3 | >99%* |
| | 8.45 (m, 1H), 8.38 (d, 1H), 8.21 (s, 1H), 7.81-7.71 (m, | [M + H]⁺ | |
| | 2H), 7.60-7.45 (m, 3H), 7.14 (d, 1H), 6.69 (d, 1H), 6.48 (s, | | |
| | 2H), 4.31-4.14 (m, 1H), 3.95-3.82 (m, 1H), 2.42-2.32 (m, | | |
| | 1H), 2.19-2.07 (m, 2H), 2.01-1.86 (m, 3H), 1.52-1.38 (m, | | |
| | 2H) | | |
| 42 | (400 MHz, DMSO-d₆) δ 9.14 (s, 1H), 8.66 (d, 1H), 8.50 | m/z 575.2 | >99%* |
| | (s, 1H), 8.32 (s, 1H), 8.24 (s, 1H), 7.75 (t, 2H), 7.61-7.44 | [M + H]⁺ | |
| | (m, 4H), 7.17 (d, 1H), 4.33-4.24 (m, 1H), 4.13 (t, 2H), | | |
| | 4.01-3.95 (m, 1H), 3.05-3.01 (m, 2H), 2.45-2.33 (m, 1H), | | |
| | 2.23-2.07 (m, 2H), 2.02-1.85 (m, 3H), 1.55-1.39 (m, 2H) | | |
| 43 | (400 MHz, CDCl₃) δ 9.06 (s, 1 H), 8.45-8.44 (m, 2 H), | m/z 504.3 | >99%* |
| | 8.16-8.15 (m, 1 H), 8.11-8.07 (m, 2 H), 7.44-7.42 (m, 1 | [M + H]⁺ | |
| | H), 4.53-4.51 (m, 1 H), 4.26 (br s, 1 H), 3.35-3.31 (m, 1 | | |
| | H), 3.17-3.14 (m, 1 H), 2.42-2.29 (m, 3 H), 2.22-2.18 (m, | | |
| | 2 H), 2.14-2.10 (m, 1 H), 1.89-1.83 (m, 2 H), 1.67 (s, 3 | | |
| | H), 1.62 (s, 3 H) | | |
| 44 | (400 MHz, CD₃OD) δ 8.95 (s, 1 H), 8.57-8.55 (m, 1 H), | m/z 522.3 | >99%* |
| | 8.54 (s, 1 H), 8.31 (br s, 1 H), 8.10-8.09 (m, 1 H), 7.62- | [M + H]⁺ | |
| | 7.60 (m, 1 H), 4.78-4.72 (m, 1 H), 4.26-4.22 (m, 1 H), | | |
| | 3.35-3.32 (m, 2 H), 2.47-2.44 (m, 1 H), 2.35-2.28 (m, 2 | | |
| | H), 2.11-2.09 (m, 2 H), 2.03-2.00 (m, 1 H), 1.78-1.73 (m, | | |
| | 2 H), 1.45 (s, 3 H), 1.41 (s, 3 H) | | |
| 45 | (400 MHz, CDCl₃) δ 9.17 (s, 1H), 8.47 (s, 1H), 8.44 (d, | m/z 524.1 | >99%* |
| | 1H), 8.16-8.10 (m, 2H), 8.02 (d, 1H), 7.94 (dd, 1H), 7.85 | [M + H]⁺ | |
| | (td, 1H), 7.77 (td, 1H), 7.65 (dt, 1H), 7.42 (dd, 1H), 4.22 | | |
| | (ddt, 1H), 4.04 (q, 1H), 2.44 (d, 1H), 2.38-2.27 (m, 2H), | | |
| | 2.22 (d, 1H), 2.13 (d, 1H), 2.10-2.02 (m, 1H), 1.54-1.38 | | |
| | (m, 2H) | | |
| 46 | (400 MHz, CDCl₃) δ 9.18 (d, 1H), 8.52 (dd, 1H), 8.48 (s, | m/z 508.2 | >99%* |
| | 1H), 8.13 (s, 1H), 8.05 (d, 1H), 7.94 (dd, 1H), 7.88-7.81 | [M + H]⁺ | |
| | (m, 2H), 7.77 (td, 1H), 7.65 (dd, 1H), 7.14 (ddd, 1H), 4.22 | | |
| | (tt, 1H), 4.04 (q, 1H), 2.44 (d, 1H), 2.40-2.28 (m, 2H), | | |
| | 2.22 (d, 1H), 2.14 (d, 1H), 2.10-2.01 (m, 1H), 1.49 (d, 2H) | | |
| 47 | (400 MHz, CDCl₃) δ 9.20 (d, 1H), 8.66 (dt, 1H), 8.49 (s, | m/z 583.1 | >99%* |
| | 1H), 8.11 (s, 1H), 7.94 (d, 1H), 7.72 (ddd, 1H), 7.57 (dt, | [M + H]⁺ | |
| | 1H), 7.42 (dd, 1H), 7.10 (d, 1H), 6.86 (d, 1H), 6.54 (d, | | |
| | 1H), 4.84 (tt, 1H), 4.20-4.06 (m, 1H), 2.54 (d, 1H), 2.30 | | |
| | (dd, 3H), 2.16 (d, 1H), 2.06 (d, 1H), 1.52 (q, 2H) | | |
| 48 | (400 MHz, CDCl₃) δ 9.20 (d, 1H), 8.65 (dt, 1H), 8.49 (s, | m/z 589.3 | >99%* |
| | 1H), 8.13-8.08 (m, 2H), 8.04 (d, 1H), 7.71 (ddt, 1H), 7.56 | [M + H]⁺ | |
| | (dt, 1H), 7.34 (ddd, 1H), 6.86 (d, 1H), 4.92-4.79 (m, 1H), | | |
| | 4.22-4.08 (m, 1H), 3.87 (dd, 2H), 2.55 (d, 1H), 2.38-2.13 | | |
| | (m, 4H), 2.10-1.99 (m, 2H), 1.63-1.50 (m, 1H), 1.50-1.37 | | |
| | (m, 1H), 1.08 (dd, 6H) | | |
| 49 | (400 MHz, CDCl₃) δ 11.93 (s, 1H), 9.19 (d, 1H), 8.46 (d, | m/z 595.2 | >99%* |
| | 1H), 8.12-8.07 (m, 2H), 8.04 (d, 1H), 7.96-7.91 (m, 1H), | [M + H]⁺ | |
| | 7.88-7.82 (m, 1H), 7.80-7.72 (m, 1H), 7.66 (d, 1H), 7.38- | | |
| | 7.31 (m, 1H), 6.86 (d, 1H), 4.22 (ddd, 1H), 4.15-4.00 (m, | | |
| | 1H), 3.87 (d, 2H), 2.49 (d, 1H), 2.40-2.27 (m, 1H), 2.20 | | |
| | (qd, 4H), 2.06 (d, 1H), 1.44 (dq, 2H), 1.09 (dd, 6H) | | |
| 50 | (400 MHz, CDCl₃) δ 9.17 (s, 1H), 8.48 (s, 1H), 8.12 (s, | m/z 582.2 | >99%* |
| | 1H), 7.94 (d, 1H), 7.65 (dddd, 2H), 7.45-7.37 (m, 2H), | [M + H]⁺ | |
| | 7.34-7.28 (m, 1H), 7.10 (d, 1H), 6.87 (d, 1H), 6.54 (s, 1H), | | |
| | 4.28 (t, 1H), 4.18-4.03 (m, 1H), 3.78-3.67 (m, 1H), 2.41 | | |
| | (d, 1H), 2.36-2.20 (m, 2H), 2.18-2.00 (m, 2H), 1.50 (q, | | |
| | 2H) | | |
| 51 | (400 MHz, CDCl₃) δ 9.13 (d, 1H), 8.44 (s, 1H), 8.10 (s, | m/z 630.1 | >99%* |
| | 1H), 7.94 (d, 1H), 7.68-7.59 (m, 2H), 7.48-7.39 (m, 2H), | [M + H]⁺ | |
| | 7.10 (d, 1H), 6.80 (d, 1H), 4.20 (ddt, 1H), 4.11-3.99 (m, | | |
| | 1H), 3.79-3.68 (m, 1H), 2.23 (d, 2H), 2.14 (s, 1H), 2.03 (d, | | |
| | 2H), 1.46 (d, 2H) | | |
| 52 | (400 MHz, CDCl₃) δ 9.12 (d, 1H), 8.44 (d, 1H), 8.14 (s, | m/z 565.1 | >99%* |
| | 1H), 8.12 (s, 1H), 7.99 (d, 1H), 7.69-7.59 (m, 2H), 7.48- | [M + H]⁺ | |
| | 7.36 (m, 3H), 4.27-4.13 (m, 1H), 4.03 (s, 1H), 3.80-3.66 | | |
| | (m, 1H), 2.29 (d, 3H), 2.12 (s, 1H), 2.03 (d, 1H), 1.58-1.41 | | |
| | (m, 2H) | | |
| 53 | (400 MHz, DMSO-d₆) δ 14.72 (s, 1H), 12.60 (s, 1H), 9.15 | m/z 532.0 | >99%* |
| | (s, 1H), 8.89 (d, 1H), 8.49 (s, 1H), 8.12 (s, 1H), 7.93 (d, | [M + H]⁺ | |
| | 1H), 7.79-7.72 (m, 2H), 7.57-7.39 (m, 3H), 6.91 (d, 1H), | | |
| | 4.35-4.22 (m, 1H), 4.06-3.93 (m, 1H), 2.45-2.30 (m, 1H), | | |
| | 2.22-2.09 (m, 2H), 2.02-1.87 (m, 3H), 1.58-1.41 (m, 2H) | | |
| 54 | (400 MHz, DMSO-d₆) δ 14.63 (s, 1H), 9.14 (s, 1H), 8.48 | m/z 590.3 | >99%* |
| | (s, 1H), 8.35-8.24 (m, 1H), 8.19 (s, 1H), 7.80-7.72 (m, | [M + H]⁺ | |
| | 2H), 7.65 (s, 1H), 7.60-7.44 (m, 3H), 7.15 (d, 1H), 4.35- | | |
| | 4.24 (m, 1H), 4.24-4.14 (m, 2H), 4.00-3.87 (m, 1H), 3.70- | | |
| | 3.59 (m, 2H), 3.21 (s, 3H), 2.25-2.09 (m, 3H), 2.05-1.86 | | |
| | (m, 3H), 1.56-1.32 (m, 2H) | | |
| 55 | (400 MHz, DMSO-d₆) δ 14.67 (s, 1H), 9.15 (s, 1H), 8.45 | m/z 302.5 | >99%* |
| | (s, 2H), 8.14 (s, 1H), 7.80-7.70 (m, 2H), 7.59-7.45 (m, | [M/2 + H]⁺ | |
| | 4H), 7.19 (d, 1H), 4.41-4.21 (m, 3H), 4.00-3.84 (m, 1H), | | |
| | 3.30-3.19 (m, 2H), 2.80-2.58 (m, 6H), 2.34-2.11 (m, 3H), | | |
| | 2.00-1.87 (m, 3H), 1.52-1.36 (m, 2H) | | |
| 56 | (400 MHz, DMSO-d₆) δ 14.63 (s, 1H), 9.14 (s, 1H), 8.60 | m/z 534.4 | >99%* |
| | (d, 1H), 8.45 (s, 1H), 8.12 (s, 1H), 7.80-7.72 (m, 2H), | [M + H]⁺ | |
| | 7.59-7.46 (m, 4H), 7.32 (t, 1H), 4.32-4.20 (m, 1H), 4.00- | | |
| | 3.85 (m, 1H), 2.40-2.24 (m, 1H), 2.21-2.04 (m, 2H), 2.04- | | |
| | 1.83 (m, 3H), 1.52-1.36 (m, 2H) | | |
| 57 | (400 MHz, DMSO-d₆) δ 8.82 (s, 1H), 8.48 (d, 1H), 8.20 | m/z 538.2 | >99%* |
| | (s, 1H), 8.15 (s, 2H), 7.68 (s, 1H), 7.49 (d, 1H), 7.18 (d, | [M + H]⁺ | |
| | 1H), 5.00 (s, 1H), 4.74-4.60 (m, 1H), 4.27-4.08 (m, 3H), | | |
| | 3.74 (s, 2H), 2.98-2.87 (m, 2H), 2.35-1.88 (m, 7H), 1.74- | | |
| | 1.59 (m, 1H), 1.56-1.41 (m, 1H), 1.03 (d, 6H) | | |
| 58 | (400 MHz, DMSO-d₆) δ 14.59 (s, 1H), 9.14 (s, 1H), 8.57 | m/z 518.0 | >99%* |
| | (d, 1H), 8.45 (s, 1H), 8.15 (s, 1H), 7.80-7.70 (m, 2H), | [M + H]⁺ | |
| | 7.58-7.45 (m, 2H), 7.40-7.27 (m, 3H), 4.34-4.21 (m, 1H), | | |
| | 3.99-3.85 (m, 1H), 2.39-2.25 (m, 1H), 2.21-2.03 (m, 2H), | | |
| | 2.01-1.83 (m, 3H), 1.52-1.38 (m, 2H) | | |
| 59 | (400 MHz, DMSO-d₆) δ 14.58 (s, 1H), 9.14 (s, 1H), 8.49 | m/z 574.7 | >99%* |
| | (s, 1H), 8.33 (d, 1H), 8.30-8.13 (m, 1H), 7.82-7.70 (m, | [M + H]⁺ | |
| | 2H), 7.60-7.43 (m, 3H), 7.35-7.27 (m, 1H), 7.20-7.12 (m, | | |
| | 1H), 4.38-4.23 (m, 1H), 4.23-4.11 (m, 2H), 4.03-3.88 (m, | | |
| | 1H), 3.70-3.62 (m, 2H), 3.23 (s, 3H), 2.27-2.08 (m, 3H), | | |
| | 2.06-1.87 (m, 3H), 1.55-1.32 (m, 2H) | | |
| 60 | (400 MHz, CD₃OD) δ 9.08 (s, 1H), 8.67 (s, 1H), 8.25 (d, | m/z 567.2 | >99%* |
| | 1H), 8.01 (d, 1H), 7.78-7.67 (m, 2H), 7.51-7.40 (m, 3H), | [M + H]⁺ | |
| | 4.32 (t, 1H), 3.99 (ddt, 1H), 3.90 (s, 3H), 2.53 (q, 1H), | | |
| | 2.44-2.34 (m, 1H), 2.29 (d, 1H), 2.04 (t, 3H), 1.75-1.62 | | |
| | (m, 1H), 1.53 (dd, 1H) | | |
| 61 | (400 MHz, CD₃OD) δ 9.11 (d, 1H), 8.71 (dd, 1H), 8.64 (d, | m/z 567.2 | >99%* |
| | 1H), 7.99 (dq, 1H), 7.79-7.72 (m, 1H), 7.36 (ddd, 1H), | [M + H]⁺ | |
| | 7.26-7.13 (m, 2H), 7.03 (s, 1H), 4.45 (dd, 2H), 3.97 (t, | | |
| | 1H), 2.44-2.26 (m, 3H), 2.16-2.01 (m, 3H), 1.66-1.41 (m, | | |
| | 2H) | | |
| 62 | (400 MHz, CD₃OD) δ 9.11 (d, 1H), 8.71 (dd, 1H), 8.68 (d, | m/z 566.2 | >99%* |
| | 1H), 8.54 (d, 1H), 8.06 (d, 1H), 7.99 (dq, 1H), 7.76 (dd, | [M + H]⁺ | |
| | 1H), 7.59 (dd, 1H), 7.02 (s, 1H), 4.49 (dt, 1H), 4.01 (ddt, | | |
| | 1H), 2.52 (q, 1H), 2.35 (qd, 2H), 2.13 (d, 1H), 2.08-1.98 | | |
| | (m, 2H), 1.68 (qd, 1H), 1.53 (dd, 1H) | | |
| 63 | (400 MHz, CD₃OD) δ 9.11 (s, 1H), 8.71 (dd, 1H), 8.68 (d, | m/z 550.2 | >99%* |
| | 1H), 8.61 (dd, 1H), 7.99 (dq, 1H), 7.78 (ddd, 2H), 7.33 | [M + H]⁺ | |
| | (ddd, 1H), 7.02 (t, 1H), 4.48 (dq, 1H), 4.07-3.94 (m, 1H), | | |
| | 2.52 (q, 1H), 2.41-2.28 (m, 2H), 2.13 (d, 1H), 2.10-1.98 | | |
| | (m, 2H), 1.76-1.61 (m, 1H), 1.57-1.44 (m, 1H) | | |
| 64 | (400 MHz, CD₃OD) δ 9.11 (s, 1H), 8.71 (dd, 1H), 8.68 (s, | m/z 562.2 | >99%* |
| | 1H), 8.25 (d, 1H), 8.00 (t, 2H), 7.76 (dd, 1H), 7.43 (dd, | [M + H]⁺ | |
| | 1H), 7.02 (t, 1H), 4.46 (ddt, 1H), 4.04-3.94 (m, 1H), 3.90 | | |
| | (s, 3H), 2.50 (q, 1H), 2.40-2.27 (m, 2H), 2.12 (d, 1H), 2.04 | | |
| | (dt, 2H), 1.66 (qd, 1H), 1.58-1.42 (m, 1H) | | |
| 65 | (400 MHz, CD₃OD) δ 9.11 (s, 1H), 8.71 (dd, 1H), 8.68 (s, | m/z 623.2 | >99%* |
| | 1H), 7.99 (dq, 1H), 7.79-7.72 (m, 2H), 7.39 (dd, 1H), 7.09 | [M + H]⁺ | |
| | (d, 1H), 7.03 (t, 1H), 4.71 (p, 1H), 4.47 (tt, 1H), 4.01-3.91 | | |
| | (m, 1H), 2.45 (d, 1H), 2.40-2.29 (m, 2H), 2.07 (dt, 3H), | | |
| | 1.65-1.45 (m, 2H), 1.35 (dd, 6H) | | |
| 66 | (400 MHz, CD₃OD) δ 9.11 (s, 1H), 8.71 (dd, 1H), 8.63 (s, | m/z 631.2 | >99%* |
| | 1H), 7.99 (dq, 1H), 7.76 (dd, 1H), 7.54 (d, 1H), 7.48 (dd, | [M + H]⁺ | |
| | 1H), 7.25-7.19 (m, 1H), 7.03 (t, 1H), 6.79 (t, 1H), 4.44 (dt, | | |
| | 1H), 3.95 (dq, 1H), 2.34 (dd, 3H), 2.15-1.95 (m, 3H), | | |
| | 1.63-1.43 (m, 2H) | | |
| 67 | (400 MHz, CD₃OD) δ 9.08 (s, 1H), 8.68 (s, 1H), 8.31 (d, | m/z 533.1 | >99%* |
| | 1H), 8.25 (d, 1H), 7.79-7.67 (m, 2H), 7.62 (dd, 1H), 7.51- | [M + H]⁺ | |
| | 7.40 (m, 2H), 4.37-4.27 (m, 1H), 4.04-3.95 (m, 1H), 2.43 | | |
| | (q, 2H), 2.06 (d, 2H), 1.76-1.61 (m, 2H), 1.52 (d, 2H) | | |
| 68 | (400 MHz, DMSO-d₆) δ 9.01 (s, 1H), 8.43 (d, 1H), 8.35 | m/z 576.4 | >99%* |
| | (s, 1H), 8.19 (s, 2H), 7.81-7.70 (m, 2H), 7.65 (s, 1H), | [M + H]⁺ | |
| | 7.58-7.51 (m, 1H), 7.50-7.45 (m, 2H), 7.17 (d, 1H), 5.03- | | |
| | 4.92 (m, 1H), 4.36-4.22 (m, 1H), 4.19-4.06 (m, 2H), 3.93 | | |
| | (s, 1H), 3.73 (s, 2H), 2.28-2.17 (m, 2H), 2.16-2.02 (m, | | |
| | 1H), 2.00-1.92 (m, 1H), 1.92-1.82 (m, 2H), 1.53-1.34 (m, | | |
| | 2H) | | |
| 69 | (400 MHz, DMSO-d₆) δ 14.71 (s, 1H), 12.66 (s, 1H), 9.13 | m/z 580.0 | >99%* |
| | (s, 1H), 8.93 (s, 1H), 8.48 (s, 1H), 8.17-8.05 (m, 1H), 7.91 | [M + H]⁺ | |
| | (s, 1H), 7.79-7.66 (m, 2H), 7.49 (d, 2H), 7.45-7.36 (m, | | |
| | 2H), 6.95-6.87 (m, 1H), 4.24-4.09 (m, 1H), 4.04-3.88 (m, | | |
| | 1H), 2.31-1.85 (m, 6H), 1.59-1.32 (m, 2H) | | |
| 70 | (400 MHz, DMSO-d₆) δ 14.65 (s, 1H), 10.80 (s, 1H), | m/z 533.3 | >99%* |
| | 9.14-9.01 (m, 2H), 8.60 (d, 1H), 8.51 (s, 1H), 8.15 (s, 1H), | [M + H]⁺ | |
| | 7.99 (s, 1H), 7.75 (d, 1H), 7.56-7.41 (m, 1H), 6.97-6.78 | | |
| | (m, 2H), 4.24-4.10 (m, 1H), 4.04-3.87 (m, 1H), 2.68-2.56 | | |
| | (m, 1H), 2.17-1.77 (m, 5H), 1.70-1.56 (m, 1H), 1.51-1.34 | | |
| | (m, 1H) | | |
| 71 | (400 MHz, DMSO-d₆) δ 9.02 (s, 1H), 8.72 (d, 1H), 8.32 | m/z 604.2 | >99%* |
| | (s, 1H), 8.20 (d, 2H), 7.81-7.69 (m, 3H), 7.58-7.45 (m, | [M + H]⁺ | |
| | 3H), 7.13 (d, 1H), 4.85 (s, 1H), 4.35-4.22 (m, 1H), 4.04- | | |
| | 3.93 (m, 1H), 3.93-3.83 (m, 2H), 2.36-2.08 (m, 3H), 2.02- | | |
| | 1.93 (m, 1H), 1.93-1.83 (m, 2H), 1.54-1.35 (m, 2H), 1.18 | | |
| | (s, 3H), 1.08 (s, 3H) | | |
| 72 | (400 MHz, DMSO-d₆) δ 9.02 (s, 1H), 8.33 (s, 1H), 8.24 | m/z 590.0 | >99%* |
| | (d, 1H), 8.19 (s, 2H), 7.80-7.72 (m, 2H), 7.66 (s, 1H), | [M + H]⁺ | |
| | 7.58-7.46 (m, 3H), 7.16 (d, 1H), 4.38-4.24 (m, 1H), 4.24- | | |
| | 4.15 (m, 2H), 4.02-3.87 (m, 1H), 3.70-3.61 (m, 2H), 3.19 | | |
| | (s, 3H), 2.27-2.07 (m, 3H), 2.04-1.85 (m, 3H), 1.55-1.30 | | |
| | (m, 2H) | | |
| 73 | (400 MHz, DMSO-d₆) δ 14.70 (s, 1H), 9.12 (s, 1H), 8.54- | m/z 624.0 | >99%* |
| | 8.40 (m, 2H), 8.26 (s, 1H), 7.79-7.64 (m, 3H), 7.58-7.36 | [M + H]⁺ | |
| | (m, 4H), 7.18 (d, 1H), 5.47 (s, 1H), 4.24-4.08 (m, 3H), | | |
| | 3.91 (s, 1H), 3.80 (s, 2H), 2.38-2.07 (m, 3H), 2.02-1.85 | | |
| | (m, 3H), 1.51-1.34 (m, 2H) | | |
| 74 | (400 MHz, DMSO-d₆) δ 14.71 (s, 1H), 9.12 (s, 1H), 8.84 | m/z 518.3 | >99%* |
| | (d, 1H), 8.44 (s, 1H), 8.07 (s, 1H), 7.82-7.72 (m, 2H), | [M + H]⁺ | |
| | 7.58-7.43 (m, 3H), 7.13 (t, 2H), 4.36-4.21 (m, 1H), 4.00- | | |
| | 3.85 (m, 1H), 2.36-2.03 (m, 3H), 2.01-1.84 (m, 3H), 1.51- | | |
| | 1.30 (m, 2H) | | |
| 75 | (400 MHz, DMSO-d₆) δ 14.80-14.03 (m, 1H), 9.15 (s, | m/z 518.3 | >99%* |
| | 1H), 8.52-8.41 (m, 2H), 8.09 (s, 1H), 7.81-7.68 (m, 2H), | [M + H]⁺ | |
| | 7.67-7.58 (m, 1H), 7.58-7.45 (m, 2H), 7.35-7.28 (m, 1H), | | |
| | 7.16-7.8 (m, 1H), 4.41-4.13 (m, 1H), 4.00-3.74 (m, 1H), | | |
| | 2.41-2.26 (m, 1H), 2.22-2.05 (m, 2H), 2.00-1.84 (m, 3H), | | |
| | 1.53-1.36 (m, 2H) | | |
| 76 | (400 MHz, DMSO-d₆) δ 14.70 (s, 1H), 9.15 (s, 1H), 8.51- | m/z 500.0 | >99%* |
| | 8.41 (m, 2H), 8.11 (s, 1H), 7.81-7.72 (m, 2H), 7.60-7.46 | [M + H]⁺ | |
| | (m, 4H), 7.30-7.21 (m, 2H), 4.33-4.21 (m, 1H), 4.02-3.84 | | |
| | (m, 1H), 2.41-2.27 (m, 1H), 2.19-2.04 (m, 2H), 2.01-1.85 | | |
| | (m, 3H), 1.53-1.39 (m, 2H) | | |
| 77 | (400 MHz, DMSO-d₆) δ 14.67 (s, 1H), 9.15 (s, 1H), 8.70 | m/z 525.0 | >99%* |
| | (d, 1H), 8.46 (s, 1H), 8.19 (s, 1H), 8.10-7.99 (m, 2H), | [M + H]⁺ | |
| | 7.82-7.71 (m, 2H), 7.59-7.46 (m, 3H), 4.34-4.19 (m, 1H), | | |
| | 4.09-3.86 (m, 1H), 2.42-2.25 (m, 1H), 2.25-2.06 (m, 2H), | | |
| | 2.04-1.88 (m, 3H), 1.55-1.36 (m, 2H) | | |
| 78 | (400 MHz, DMSO-d₆) δ 14.57 (s, 1H), 8.97 (s, 1H), 8.54 | m/z 538.2 | >99%* |
| | (d, 1H), 8.35 (s, 1H), 8.20 (s, 1H), 7.73 (d, 1H), 7.50 (dd, | [M + H]⁺ | |
| | 1H), 7.20 (d, 1H), 5.51 (s, 1H), 4.74-4.61 (m, 1H), 4.28- | | |
| | 4.10 (m, 3H), 3.88-3.78 (m, 2H), 2.92 (d, 2H), 2.40-2.15 | | |
| | (m, 3H), 2.11-1.99 (m, 2H), 1.99-1.86 (m, 2H), 1.79-1.59 | | |
| | (m, 1H), 1.54-1.41 (m, 1H), 1.03 (d, 6H) | | |
| 79 | (400 MHz, DMSO-d₆) δ 14.09 (s, 1H), 8.96 (s, 1H), 8.33 | m/z 534.4 | >99%* |
| | (s, 1H), 8.20-8.08 (m, 2H), 7.53-7.43 (m, 2H), 7.38 (d, | [M + H]⁺ | |
| | 1H), 4.74-4.56 (m, 1H), 4.20-4.06 (m, 1H), 3.99-3.89 (m, | | |
| | 1H), 2.92 (d, 2H), 2.34-2.18 (m, 2H), 2.18-2.05 (m, 2H), | | |
| | 2.02-1.84 (m, 3H), 1.73-1.56 (m, 1H), 1.56-1.39 (m, 1H), | | |
| | 1.03 (d, 6H), 0.78-0.65 (m, 4H) | | |
| 80 | (400 MHz, DMSO-d₆) δ 14.72 (s, 1H), 9.19 (s, 1H), 8.72 | m/z 519.0 | >99%* |
| | (s, 1H), 8.57 (d, 1H), 8.50 (s, 1H), 8.20-8.04 (m, 2H), | [M + H]⁺ | |
| | 7.87-7.78 (m, 1H), 7.41-7.28 (m, 3H), 4.90-4.73 (m, 1H), | | |
| | 4.06-3.84 (m, 1H), 2.41-2.19 (m, 2H), 2.14-2.01 (m, 2H), | | |
| | 1.97-1.85 (m, 2H), 1.56-1.35 (m, 2H) | | |
| 81 | (400 MHz, CD₃OD) δ 9.13 (s, 1H), 8.67 (s, 1H), 8.40-8.17 | m/z 525.4 | >99%* |
| | (m, 1H), 8.08 (d, 1H), 8.01-7.95 (m, 1H), 7.91-7.82 (m, | [M + H]⁺ | |
| | 2H), 7.36 (s, 1H), 7.30-7.16 (m, 2H), 4.33-4.22 (m, 1H), | | |
| | 4.08-3.92 (m, 1H), 2.48-2.33 (m, 3H), 2.21-2.10 (m, 1H), | | |
| | 2.10-1.99 (m, 2H), 1.68-1.42 (m, 2H) | | |
| 82 | Data provided above | | |
| 83 | (400 MHz, DMSO-d₆) δ 14.66 (m, 1H), 9.13 (s, 1H), 8.47 | m/z 602.2 | >99% |
| | (s, 1H), 8.18 (d, 1H), 7.74 (t, 2H), 7.58-7.41 (m, 5H), 7.11 | [M + H]⁺ | |
| | (d, 1H), 5.15-5.06 (m, 1H), 4.35-4.21 (m, 1H), 3.94-3.68 | (Rt = 2.026 | |
| | (m, 5H), 2.37-2.05 (m, 5H), 1.99-1.90 (m, 3H), 1.51-1.40 | min) | |
| | (m, 2H) | | |
| 84 | (400 MHz, DMSO-d₆) δ 14.66 (s, 1H), 9.13 (s, 1H), 8.47 | m/z 602.2 | 96% |
| | (s, 1H), 8.18 (d, 1H), 7.74 (t, 2H), 7.59-7.41 (m, 5H), 7.12 | [M + H]⁺ | |
| | (d, 1H), 5.13-5.03 (m, 1H), 4.37-4.22 (m, 1H), 3.95-3.86 | (Rt = 2.203 | |
| | (m, 1H), 3.85-3.74 (m, 4H), 2.26-2.08 (m, 4H), 2.04-1.95 | min) | |
| | (m, 2H), 1.91-1.84 (m, 2H), 1.53-1.39 (m, 2H) | | |
| 85 | (400 MHz, DMSO-d₆) δ 9.14 (s, 1H), 8.60-8.44 (m, 2H), | m/z 560.5 | >99%* |
| | 8.34 (s, 1H), 7.64 (t, 1H), 7.51 (d, 1H), 7.34 (d, 4H), 7.17 | [M + H]⁺ | |
| | (t, 1H), 4.21-4.11 (m, 3H), 3.92-3.83 (m, 1H), 3.63-3.58 | | |
| | (m, 3H), 2.38-1.98 (m, 4H), 1.93-1.82 (m, 2H), 1.55-1.37 | | |
| | (m, 2H) | | |
| 86 | (400 MHz, DMSO-d₆) δ 14.69 (s, 1H), 9.09 (s, 1H), 8.58 | m/z 576.5 | >99%* |
| | (s, 1H), 8.42 (s, 1H), 8.07 (s, 1H), 7.62 (t, 1H), 7.59-7.52 | [M + H]⁺ | |
| | (m, 2H), 7.50 (d, 1H), 7.40-7.27 (m, 2H), 7.16 (t, 1H), | | |
| | 4.91-4.63 (m, 1H), 4.24-4.02 (m, 3H), 3.97-3.73 (m, 1H), | | |
| | 3.62 (d, 2H), 2.44-1.82 (m, 6H), 1.55-1.37 (m, 2H) | | |
| 87 | (400 MHz, DMSO-d₆) δ 14.64 (s, 1H), 9.20-8.94 (m, 2H), | m/z 585.5 | >99%* |
| | 8.58 (s, 1H), 8.47 (s, 1H), 8.20-7.88 (m, 2H), 7.79-7.69 | [M + H]⁺ | |
| | (m, 1H), 7.63 (t, 1H), 7.49 (d, 1H), 7.26 (d, 1H), 7.18 (t, | | |
| | 1H), 5.23-4.99 (m, 1H), 4.14-3.41 (m, 6H), 2.84-2.60 (m, | | |
| | 1H), 2.39-1.61 (m, 8H), 1.52-1.20 (m, 1H) | | |
| 88 | (400 MHz, DMSO-d₆) δ 14.67 (s, 1H), 9.14 (s, 1H), 8.45 | m/z 628.4 | >99% |
| | (s, 1H), 8.22 (d, 1H), 8.08 (s, 1H), 7.80-7.70 (m, 2H), | [M + H]⁺ | |
| | 7.58-7.41 (m, 4H), 7.28 (d, 1H), 5.27-5.16 (m, 1H), 4.35- | (Rt = 3.862 | |
| | 4.22 (m, 1H), 4.03-3.78 (m, 1H), 2.25-2.13 (m, 2H), 2.11- | min) | |
| | 2.04 (m, 1H), 2.00-1.94 (m, 1H), 1.92-1.84 (m, 2H), 1.53- | | |
| | 1.42 (m, 1H), 1.39 (d, 3H), 1.36-1.31 (m, 1H) | | |
| 89 | (400 MHz, DMSO-d₆) δ 14.67 (s, 1H), 9.14 (s, 1H), 8.46 | m/z 628.4 | >99% |
| | (s, 1H), 8.22 (d, 1H), 8.08 (s, 1H), 7.80-7.70 (m, 2H), | [M + H]⁺ | |
| | 7.57-7.45 (m, 3H), 7.42 (d, 1H), 7.28 (d, 1H), 5.33-5.10 | (Rt = 4.053 | |
| | (m, 1H), 4.34-4.19 (m, 1H), 4.02-3.76 (m, 1H), 2.37-1.98 | min) | |
| | (m, 4H), 1.96-1.87 (m, 2H), 1.51-1.38 (m, 2H), 1.35 (d, | | |
| | 3H) | | |
| 90 | Data provided above | | |
| 91 | (400 MHz, DMSO-d₆) δ 14.63 (s, 1H), 9.09 (s, 1H), 9.06- | m/z 611.2 | >99% |
| | 8.94 (m, 1H), 8.59 (s, 1H), 8.49 (s, 1H), 8.23-7.94 (m, | [M + H]⁺ | |
| | 2H), 7.79-7.63 (m, 2H), 7.58-7.46 (m, 2H), 7.34-7.22 (m, | (Rt = 4.290 | |
| | 1H), 5.44-5.27 (m, 1H), 4.15-3.98 (m, 1H), 3.98-3.81 (m, | min) | |
| | 1H), 2.89-2.61 (m, 1H), 2.32-1.70 (m, 5H), 1.71-1.08 (m, | | |
| | 5H) | | |
| 92 | (400 MHz, DMSO-d₆) δ 14.74 (s, 1H), 9.25-9.02 (m, 2H), | m/z 611.2 | >99% |
| | 8.73-8.63 (m, 1H), 8.56 (s, 1H), 8.15 (s, 1H), 8.12-7.98 | [M + H]⁺ | |
| | (m, 1H), 7.81 (s, 1H), 7.74 (t, 1H), 7.64-7.53 (m, 2H), | (Rt = 6.067 | |
| | 7.33 (t, 1H), 5.49-5.33 (m, 1H), 4.16-3.83 (m, 2H), 2.92- | min) | |
| | 2.73 (m, 1H), 2.41-1.72 (m, 6H), 1.60-1.24 (m, 4H) | | |
| 93 | (400 MHz, DMSO-d₆) δ 9.16 (s, 1H), 8.53 (d, 1H), 8.44 | m/z 539.6 | >99%* |
| | (s, 1H), 8.16 (d, 1H), 8.01 (d, 1H), 7.98-7.94 (m, 1H), | [M + H]⁺ | |
| | 7.92-7.86 (m, 2H), 7.37-7.31 (m, 3H), 4.39-4.19 (m, 1H), | | |
| | 4.01-3.76 (m, 1H), 2.32-2.25 (m, 1H), 2.22 (d, 3H), 2.20- | | |
| | 2.15 (m, 1H), 2.09-2.02 (m, 2H), 1.90-1.83 (m, 2H), 1.53- | | |
| | 1.34 (m, 2H) | | |
| 94 | (400 MHz, DMSO-d₆) δ 14.52 (s, 1H), 9.08 (d, 1H), 8.95 | m/z 521.1 | >99%* |
| | (s, 1H), 8.62 (d, 1H), 8.39 (s, 1H), 8.10 (s, 1H), 8.03 (d, | [M + H]⁺ | |
| | 1H), 7.76 (dd, 1H), 4.71-4.60 (m, 1H), 4.32-4.17 (m, 1H), | | |
| | 3.85 (t, 1H), 3.81-3.74 (m, 1H), 3.70-3.60 (m, 1H), 3.43- | | |
| | 3.37 (m, 1H), 3.11-2.99 (m, 2H), 2.69-2.56 (m, 1H), 2.38- | | |
| | 2.24 (m, 1H), 2.13-1.99 (m, 3H), 1.98-1.84 (m, 5H), 1.74- | | |
| | 1.63 (m, 2H), 1.62-1.53 (m, 1H) | | |
| 95 | Data provided above | | |
| 96 | Data provided above | | |
| 97 | (400 MHz, CD₃OD) δ 9.11 (d, 1H), 8.71 (dd, 1H), 8.67 (d, | m/z 581.2 | >99%* |
| | 1H), 7.99 (dt, 1H), 7.80 (d, 1H), 7.75 (ddd, 1H), 7.31 (dt, | [M + H]⁺ | |
| | 1H), 7.02 (t, 1H), 6.87-6.84 (m, 1H), 4.45 (tt, 1H), 4.02 (tt, | | |
| | 1H), 2.44 (q, 1H), 2.39-2.26 (m, 2H), 2.11 (d, 1H), 2.08- | | |
| | 2.00 (m, 2H), 1.68-1.42 (m, 2H) | | |
| 98 | (400 MHz, CD₃OD) δ 9.09 (s, 1H), 8.63 (s, 1H), 7.87 (dd, | m/z 525.1 | >99%* |
| | 1H), 7.73 (dtd, 2H), 7.56-7.36 (m, 4H), 4.33 (t, 1H), 3.99 | [M + H]⁺ | |
| | (ddt, 1H), 2.50-2.28 (m, 3H), 2.08 (q, 3H), 1.68-1.45 (m, | | |
| | 2H) | | |
| 99 | (400 MHz, CD₃OD) δ 9.14 (s, 1H), 8.75 (d, 1H), 8.71 (q, | m/z 576.2 | >99%* |
| | 1H), 8.62 (dd, 1H), 8.07 (dd, 1H), 8.00-7.94 (m, 1H), | [M + H]⁺ | |
| | 7.91-7.83 (m, 2H), 7.79 (dd, 1H), 7.33 (ddd, 1H), 4.34 (tt, | | |
| | 1H), 4.08-3.93 (m, 1H), 2.54 (q, 1H), 2.44-2.24 (m, 2H), | | |
| | 2.14 (d, 1H), 2.02 (t, 2H), 1.68 (qd, 1H), 1.50 (q, 1H) | | |
| 100 | (400 MHz, CD₃OD) δ 9.08 (s, 1H), 8.75 (d, 1H), 8.67 (s, | m/z 533.2 | >99%* |
| | 1H), 8.55 (d, 1H), 8.34 (s, 1H), 8.18 (s, 1H), 7.78-7.65 (m, | [M + H]⁺ | |
| | 3H), 7.52-7.36 (m, 2H), 6.89 (t, 2H), 4.34 (dd, 1H), 4.04 | | |
| | (ddt, 1H), 2.56 (q, 1H), 2.41 (td, 1H), 2.30 (d, 1H), 2.07 | | |
| | (dd, 3H), 1.79-1.64 (m, 1H), 1.58-1.43 (m, 1H) | | |
| 101 | (400 MHz, CD₃OD) δ 9.14 (d, 1H), 8.71 (d, 1H), 8.70 (d, | m/z 593.1 | >99%* |
| | 1H), 8.07 (d, 1H), 8.00-7.93 (m, 1H), 7.91-7.82 (m, 2H), | [M + H]⁺ | |
| | 7.36 (ddd, 1H), 7.27-7.13 (m, 2H), 4.40-4.27 (m, 1H), | | |
| | 3.98 (d, 1H), 2.44-2.37 (m, 2H), 2.36-2.25 (m, 1H), 2.15 | | |
| | (d, 1H), 2.04 (d, 2H), 1.65-1.43 (m, 2H) | | |
| 102 | (400 MHz, DMSO-d₆) δ 14.62 (s, 1H), 9.13 (s, 1H), 8.46 | m/z 562.1 | >99% |
| | (s, 1H), 8.19 (d, 2H), 7.77-7.68 (m, 2H), 7.56 (d, 1H), | [M + H]⁺ | |
| | 7.54-7.42 (m, 3H), 7.09 (d, 1H), 5.23 (d, 1H), 4.25-4.13 | | |
| | (m, 1H), 4.06-3.94 (m, 1H), 3.81 (s, 3H), 3.72-3.62 (m, | | |
| | 1H), 2.44-2.28 (m, 1H), 2.19-2.09 (m, 1H), 1.94-1.80 (m, | | |
| | 2H), 1.71-1.52 (m, 1H), 1.49-1.34 (m, 1H) | | |
| 103 | (400 MHz, DMSO-d₆) δ 14.63 (s, 1H), 9.12 (s, 1H), 8.94 | m/z 533.2 | >99% |
| | (d, 1H), 8.59 (d, 1H), 8.53 (s, 1H), 7.98 (d, 1H), 7.78-7.67 | [M + H]⁺ | |
| | (m, 4H), 7.55-7.45 (m, 2H), 5.10 (d, 1H), 4.49-4.40 (m, | | |
| | 1H), 4.07-3.94 (m, 1H), 3.79-3.71 (m, 1H), 2.41-2.32 (m, | | |
| | 1H), 2.16-2.08 (m, 1H), 1.90-1.79 (m, 3H), 1.51-1.39 (m, | | |
| | 1H) | | |
| 104 | (400 MHz, CD₃OD) δ 9.07 (s, 1H), 8.57 (s, 1H), 8.27 (s, | m/z 576.1 | >99% |
| | 1H), 7.78 (d, 1H), 7.72 (q, 2H), 7.48-7.35 (m, 3H), 7.09 | [M + H]⁺ | |
| | (d, 1H), 4.33-4.11 (m, 2H), 3.89 (s, 3H), 3.86 (s, 1H), 2.77 | (R t= 2.83 min) | |
| | (s, 1H), 2.22 (d, 1H), 1.99 (d, 3H), 1.93-1.80 (m, 1H), 1.73 | | |
| | (s, 2H) | | |
| 105 | (400 MHz, CD₃OD) δ 9.07 (s, 1H), 8.57 (s, 1H), 8.27 (s, | m/z 576.1 | >99% |
| | 1H), 7.78 (d, 1H), 7.72 (q, 2H), 7.48-7.35 (m, 3H), 7.09 | [M + H]⁺ | |
| | (d, 1H), 4.33-4.11 (m, 2H), 3.89 (s, 3H), 3.86 (s, 1H), 2.77 | (Rt = 4.78 min) | |
| | (s, 1H), 2.22 (d, 1H), 1.99 (d, 3H), 1.93-1.80 (m, 1H), 1.73 | | |
| | (s, 2H) | | |
| 106 | (400 MHz, DMSO-d₆) δ 14.69 (s, 1H), 9.15 (s, 1H), 8.46 | m/z 576.5 | >99% |
| | (s, 1H), 8.27 (d, 1H), 8.10 (s, 1H), 7.80-7.72 (m, 2H), 7.56 | [M + H]⁺ | |
| | (t, 1H), 7.53-7.49 (m, 2H), 7.49-7.45 (m, 1H), 7.12 (d, | (Rt = 1.432 | |
| | 1H), 4.49-4.33 (m, 1H), 4.16-4.03 (m, 1H), 3.84-3.77 (m, | min) | |
| | 4H), 3.15 (s, 3H), 2.45-2.36 (m, 1H), 2.33-2.20 (m, 2H), | | |
| | 2.16-2.04 (m, 2H), 1.78-1.66 (m, 1H) | | |
| 107 | (400 MHz, DMSO-d₆) 14.69 (s, 1H), 9.15 (s, 1H), 8.46 (s, | m/z 576.5 | >99% |
| | 1H), 8.27 (d, 1H), 8.11 (s, 1H), 7.83-7.72 (m, 2H), 7.60- | [M + H]⁺ | |
| | 7.45 (m, 4H), 7.12 (d, 1H), 4.49-4.37 (m, 1H), 4.17-4.03 | (Rt = 3.319 | |
| | (m, 1H), 3.86-3.80 (m, 4H), 3.15 (s, 3H), 2.47-2.37 (m, | min) | |
| | 1H), 2.33-2.19 (m, 2H), 2.18-2.04 (m, 2H), 1.80-1.66 (m, | | |
| | 1H) | | |
| 108 | (400 MHz, DMSO-d₆) δ 15.26-14.23 (m, 1H), 9.25-9.14 | m/z 569.2 | >99%* |
| | (m, 1H), 8.51 (s, 1H), 8.19 (d, 2H), 8.16-8.06 (m, 1H), | [M + H]⁺ | |
| | 7.99-7.89 (m, 2H), 7.87-7.82 (m, 1H), 7.62-7.55 (m, 1H), | | |
| | 7.45 (dd, 1H), 7.10 (d, 1H), 5.46-5.26 (m, 1H), 4.24-4.15 | | |
| | (m, 1H), 4.13-4.01 (m, 1H), 3.80 (s, 3H), 3.69-3.62 (m, | | |
| | 1H), 2.41-2.32 (m, 1H), 2.26-2.16 (m, 1H), 1.94-1.78 (m, | | |
| | 2H), 1.69-1.58 (m, 1H), 1.54-1.30 (m, 1H) | | |
| 109 | (400 MHz, CD₃OD) δ 9.04 (s, 1H), 8.55 (s, 1H), 7.84-7.76 | m/z 606.2 | 86% |
| | (m, 1H), 7.62 (s, 1H), 7.44 (ddd, 1H), 7.14-7.03 (m, 2H), | [M + H]⁺ | |
| | 6.95 (t, 1H), 4.21 (t, 1H), 4.00 (d, 1H), 3.89 (s, 3H), 3.88 | (Rt = 2.70 min) | |
| | (s, 3H), 2.73 (s, 1H), 2.18 (d, 1H), 2.06 (d, 1H), 1.98 (s, | | |
| | 1H), 1.80 (s, 2H), 1.65 (s, 1H) | | |
| 110 | (400 MHz, CD₃OD) δ 9.04 (s, 1H), 8.55 (s, 1H), 7.84-7.76 | m/z 606.2 | 75% |
| | (m, 1H), 7.62 (s, 1H), 7.44 (ddd, 1H), 7.14-7.03 (m, 2H), | [M + H]⁺ | |
| | 6.95 (t, 1H), 4.21 (t, 1H), 4.00 (d, 1H), 3.89 (s, 3H), 3.88 | (Rt = 5.30 min) | |
| | (s, 3H), 2.73 (s, 1H), 2.18 (d, 1H), 2.06 (d, 1H), 1.98 (s, | | |
| | 1H), 1.80 (s, 2H), 1.65 (s, 1H) | | |
| 111 | (400 MHz, CD₃OD) δ 9.05-8.91 (m, 3H), 8.85 (s, 1H), | m/z 535.0 | 96% |
| | 7.76 (d, 1H), 7.52-7.47 (m, 1H), 7.47-7.40 (m, 1H), 7.11 | [M + H]⁺ | |
| | (d, 1H), 5.14-5.02 (m, 1H), 4.72-4.61 (m, 1H), 4.54-4.42 | (Rt = 5.784 | |
| | (m, 1H), 3.92 (s, 3H), 3.08-2.98 (m, 2H), 2.67-2.46 (m, | min) | |
| | 2H), 2.39-2.21 (m, 3H), 2.11-2.02 (m, 1H), 1.97-1.89 (m, | | |
| | 1H), 1.12-1.07 (m, 6H) | | |
| 112 | (400 MHz, CD₃OD) δ 9.02-8.82 (m, 4H), 7.78-7.72 (m, | m/z 535.0 | 98% |
| | 1H), 7.55-7.47 (m, 1H), 7.47-7.40 (m, 1H), 7.11 (d, 1H), | [M + H]⁺ | |
| | 5.17-5.02 (m, 1H), 4.73-4.61 (m, 1H), 4.48-4.42 (m, 1H), | (Rt = 3.719 | |
| | 3.92 (s, 3H), 3.06-2.93 (m, 2H), 2.65-2.47 (m, 2H), 2.39- | min) | |
| | 2.22 (m, 3H), 2.10-2.02 (m, 1H), 1.98-1.87 (m, 1H), 1.12- | | |
| | 1.05 (m, 6H) | | |
| 113 | (400 MHz, DMSO-d₆) δ 14.70 (s, 1H), 9.14 (s, 1H), 9.08 | m/z 531.1 | >99% |
| | (d, 1H), 8.67 (dd, 1H), 8.51 (s, 1H), 8.11 (s, 1H), 7.81- | [M + H]⁺ | |
| | 7.72 (m, 3H), 7.60-7.48 (m, 3H), 4.49-4.38 (m, 1H), 4.30- | (Rt = 0.839 | |
| | 4.14 (m, 1H), 3.87-3.75 (m, 1H), 3.14 (s, 3H), 2.78-2.66 | min) | |
| | (m, 1H), 2.30-2.20 (m, 2H), 2.09-1.95 (m, 2H), 1.95-1.85 | | |
| | (m, 1H) | | |
| 114 | (400 MHz, DMSO-d₆) δ 14.71 (s, 1H), 9.23-9.08 (m, 1H), | m/z 531.2 | >99% |
| | 9.01-8.85 (m, 1H), 8.75-8.58 (m, 1H), 8.56-8.50 (m, 1H), | [M + H]⁺ | |
| | 8.21-8.06 (m, 1H), 7.84-7.66 (m, 3H), 7.62-7.44 (m, 3H), | (Rt = 5.208 | |
| | 4.54-4.23 (m, 1H), 4.23-3.95 (m, 1H), 3.51-3.42 (m, 1H), | min) | |
| | 3.25 (s, 3H), 2.67-2.56 (m, 1H), 2.40-2.28 (m, 1H), 2.27- | | |
| | 2.12 (m, 1H), 2.11-1.93 (m, 2H), 1.74-1.60 (m, 1H) | | |
| 115 | (400 MHz, DMSO-d₆) δ 14.71 (s, 1H), 9.20-9.06 (m, 2H), | m/z 531 | >99% |
| | 8.67 (dd, 1H), 8.53 (s, 1H), 8.11 (s, 1H), 7.83-7.71 (m, | [M + H]⁺ | |
| | 3H), 7.59-7.45 (m, 3H), 4.43-4.28 (m, 1H), 4.17-4.01 (m, | (Rt = 2.554 | |
| | 1H), 3.51-3.39 (m, 1H), 3.24 (s, 3H), 2.69-2.55 (m, 1H), | min) | |
| | 2.36-2.30 (m, 1H), 2.21-2.12 (m, 1H), 2.06-1.94 (m, 2H), | | |
| | 1.70-1.59 (m, 1H) | | |
| 116 | (400 MHz, DMSO-d₆) δ 14.68 (s, 1H), 9.14 (s, 1H), 9.08 | m/z 547.1 | >99% |
| | (d, 1H), 8.61 (d, 1H), 8.51 (s, 1H), 8.19 (s, 1H), 8.03-7.98 | [M + H]⁺ | |
| | (m, 1H), 7.81-7.73 (m, 3H), 7.60-7.47 (m, 2H), 4.53-4.36 | (Rt = 0.842 | |
| | (m, 1H), 4.30-4.14 (m, 1H), 3.85-3.77 (m, 1H), 3.14 (s, | min) | |
| | 3H), 2.82-2.64 (m, 1H), 2.35-2.18 (m, 2H), 2.11-1.82 (m, | | |
| | 3H) | | |
| 117 | (400 MHz, DMSO-d₆) δ 9.13-8.89 (m, 4H), 8.70 (s, 1H), | m/z 520.1 | >99% |
| | 8.61 (d, 1H), 8.03 (d, 1H), 7.80-7.73 (m, 1H), 7.55 (s, 1H), | [M + H]⁺ | |
| | 4.81-4.69 (m, 1H), 4.52-4.36 (m, 1H), 3.88 (s, 1H), 3.36 | (Rt = 1.608 | |
| | (s, 3H), 2.97-2.81 (m, 2H), 2.75-2.62 (m, 1H), 2.34-2.04 | min) | |
| | (m, 5H), 1.93-1.82 (m, 1H), 1.05 (d, 3H), 1.01 (d, 3H) | | |
| 118 | (400 MHz, DMSO-d₆) δ 9.27 (s, 1H), 9.11 (s, 1H), 8.92 | m/z 583.0 | >99% |
| | (d, 1H), 8.58 (d, 1H), 8.53 (s, 1H), 8.09 (t, 1H), 7.98 (s, | [M + H]⁺ | |
| | 1H), 7.77-7.67 (m, 3H), 7.53-7.42 (m, 2H), 5.13-5.06 (m, | (Rt = 1.439 | |
| | 1H), 4.48-4.32 (m, 1H), 4.06-3.93 (m, 1H), 3.81-3.68 (m, | min) | |
| | 1H), 2.41-2.27 (m, 1H), 2.21-2.08 (m, 1H), 1.92-1.73 (m, | | |
| | 3H), 1.50-1.38 (m, 1H) | | |
| 119 | (400 MHz, DMSO-d₆) δ 14.69 (s, 1H), 9.15 (s, 1H), 8.56- | m/z 606.2 | >99% |
| | 8.44 (m, 2H), 8.23 (s, 1H), 7.81-7.72 (m, 2H), 7.68 (s, | [M + H]⁺ | |
| | 1H), 7.60-7.46 (m, 3H), 7.19 (d, 1H), 5.42 (s, 1H), 4.54- | (Rt = 3.629 | |
| | 4.41 (m, 1H), 4.23-4.05 (m, 3H), 3.86-3.77 (m, 3H), 3.15 | min) | |
| | (s, 3H), 2.47-2.38 (m, 1H), 2.31-2.12 (m, 4H), 1.73-1.58 | | |
| | (m, 1H) | | |
| 120 | (400 MHz, DMSO-d₆) δ 14.03 (s, 1H), 9.14 (s, 1H), 8.50- | m/z 534.2 | >99% |
| | 8.38 (m, 2H), 8.27-8.10 (m, 1H), 7.80-7.64 (m, 2H), 7.57- | [M + H]⁺ | |
| | 7.44 (m, 2H), 7.44-7.38 (m, 1H), 7.37-7.26 (m, 2H), 5.25 | (Rt = 2.657 | |
| | (s, 1H), 4.29-4.13 (m, 1H), 4.10-3.93 (m, 1H), 3.81-3.65 | min) | |
| | (m, 1H), 2.43-2.27 (m, 1H), 2.23-2.07 (m, 1H), 1.94-1.81 | | |
| | (m, 2H), 1.70-1.54 (m, 1H), 1.52-1.36 (m, 1H) | | |
| 121 | (400 MHz, DMSO-d₆) δ 14.43 (s, 1H), 8.96 (s, 1H), 8.48- | m/z 496.5 | >99% |
| | 8.42 (m, 1H), 8.29 (s, 2H), 7.49-7.41 (m, 1H), 7.39-7.28 | [M + H]⁺ | |
| | (m, 2H), 5.16 (d, 1H), 4.45-4.33 (m, 1H), 4.24-4.12 (m, | (Rt = 0.956 | |
| | 1H), 4.07-3.95 (m, 1H), 2.98-2.89 (m, 2H), 2.35-2.26 (m, | min) | |
| | 2H), 2.00-1.92 (m, 2H), 1.87-1.82 (m, 1H), 1.70-1.59 (m, | | |
| | 2H), 1.04 (d, 6H) | | |
| 122 | (400 MHz, DMSO-d₆) δ 14.66 (s, 1H), 9.15 (s, 1H), 8.55 | m/z 548.2 | 98% |
| | (d, 1H), 8.44 (s, 1H), 8.17 (s, 1H), 7.76 (s, 2H), 7.60-7.45 | [M + H]⁺ | |
| | (m, 2H), 7.35 (s, 3H), 4.49-4.36 (m, 1H), 4.18-4.03 (m, | (Rt = 1.416 | |
| | 1H), 3.81 (s, 1H), 3.16 (s, 3H), 2.45-2.33 (m, 1H), 2.33- | min) | |
| | 2.19 (m, 2H), 2.19-2.06 (m, 2H), 1.71-1.61 (m, 1H) | | |
| 123 | (400 MHz, DMSO-d₆) δ 9.07 (s, 1H), 8.50 (s, 1H), 8.41 (s, | m/z 590.5 | >99% |
| | 1H), 8.30 (s, 1H), 7.66-7.59 (m, 1H), 7.49 (dd, 1H), 7.45 | [M + H]⁺ | |
| | (s, 1H), 7.41-7.30 (m, 4H), 7.16 (t, 1H), 4.79-4.63 (m, | (Rt = 0.731 | |
| | 1H), 4.40-4.27 (m, 1H), 4.19-3.98 (m, 3H), 3.84-3.69 (m, | min) | |
| | 1H), 3.64-3.56 (m, 2H), 3.24-3.12 (m, 1H), 3.09-2.86 (m, | | |
| | 2H), 2.45-2.18 (m, 3H), 2.10-2.00 (m, 1H), 1.99-1.85 (m, | | |
| | 1H), 1.70-1.58 (m, 1H) | | |
| 124 | (400 MHz, DMSO-d₆) δ 9.07 (s, 1H), 8.54 (s, 1H), 8.41 (s, | m/z 606.5 | >99% |
| | 1H), 8.30 (s, 1H), 7.66-7.53 (m, 3H), 7.49 (d, 1H), 7.45 (s, | [M + H]⁺ | |
| | 1H), 7.38-7.28 (m, 2H), 7.16 (t, 1H), 4.81-4.62 (m, 1H), | (Rt = 1.339 | |
| | 4.39-4.26 (m, 1H), 4.20-3.99 (m, 3H), 3.81-3.68 (m, 1H), | min) | |
| | 3.66-3.56 (m, 2H), 3.23-3.13 (m, 1H), 3.04-2.84 (m, 2H), | | |
| | 2.41-2.33 (m, 1H), 2.34-2.14 (m, 2H), 2.09-1.86 (m, 2H), | | |
| | 1.68-1.58 (m, 1H) | | |
| 125 | (400 MHz, CD₃OD) δ 9.05 (s, 1H), 8.62 (s, 1H), 7.83 (s, | m/z 548.3 | >99% |
| | 1H), 7.77-7.66 (m, 2H), 7.52-7.36 (m, 3H), 7.27-7.15 (m, | [M + H]⁺ | |
| | 2H), 4.32 (t, 1H), 4.18-4.06 (m, 1H), 3.92-3.81 (m, 1H), | (Rt = 2.641 | |
| | 2.63-2.49 (m, 1H), 2.30 (s, 3H), 2.28-2.22 (m, 1H), 2.14- | min) | |
| | 1.97 (m, 2H), 1.82-1.69 (m, 1H), 1.65-1.49 (m, 1H) | | |
| 126 | (400 MHz, CD₃OD) δ 9.07 (s, 1H), 8.67 (d, 1H), 8.61 (s, | m/z 616.2 | N/A |
| | 1H), 7.72 (dtd, 2H), 7.50-7.35 (m, 3H), 7.27-7.11 (m, 2H), | [M + H]⁺ | |
| | 4.25 (t, 2H), 3.86 (t, 1H), 2.75 (d, 1H), 2.25 (d, 1H), 1.99 | | |
| | (s, 2H), 1.87 (d, 2H), 1.82-1.58 (m, 2H) | | |
| 127 | (400 MHz, CD₃OD) δ 9.10 (s, 1H), 8.62 (s, 1H), 8.40-8.30 | m/z 533.1 | >99% |
| | (m, 1H), 7.80-7.69 (m, 2H), 7.52-7.39 (m, 3H), 7.29-7.16 | [M + H]⁺ | |
| | (m, 2H), 4.12-4.01 (m, 1H), 3.94-3.82 (m, 1H), 3.65 (t, | (Rt = 1.177 | |
| | 1H), 2.61-2.45 (m, 1H), 2.30-2.19 (m, 1H), 2.15-1.96 (m, | min) | |
| | 2H), 1.85-1.71 (m, 1H), 1.67-1.50 (m, 1H) | | |
| 128 | (400 MHz, DMSO-d₆) δ 14.68 (s, 1H), 9.21-9.11 (m, 2H), | m/z 567.5 | N/A |
| | 8.67 (dd, 1H), 8.51 (s, 1H), 8.11 (s, 1H), 7.83-7.70 (m, | [M + H]⁺ | |
| | 3H), 7.58-7.45 (m, 3H), 6.69 (t, 1H), 4.81-4.73 (m, 1H), | | |
| | 4.53-4.44 (m, 1H), 4.33-4.18 (m, 1H), 2.83-2.73 (m, 1H), | | |
| | 2.46-2.42 (m, 1H), 2.22-2.04 (m, 3H), 2.01-1.93 (m, 1H) | | |
| 129 | (400 MHz, CDCl₃) δ 9.15 (s, 1 H), 8.55 (br s, 1 H), 8.46- | m/z 532.2 | 100% |
| | 8.44 (m, 1 H), 8.15 (s, 2 H), 8.08-8.06 (m, 1 H), 7.68-7.62 | [M + H]⁺ | |
| | (m, 2 H), 7.44-7.43 (m, 1 H), 7.41-7.38 (m, 1 H), 7.32- | (Rt = 1.640 | |
| | 7.30 (m, 1 H), 4.59-4.55 (m, 1 H), 4.38-4.33 (m, 1 H), | min) | |
| | 3.23-3.21 (m, 1 H), 3.11-3.08 (m, 1 H), 2.95-2.88 (m, 1 | | |
| | H), 2.43 (br s, 3 H), 2.27-2.22 (m, 3 H) | | |
| 130 | (400 MHz, CDCl₃) δ 9.16 (s, 1 H), 8.55 (br s, 1 H), 8.46- | m/z 532.2 | 99% |
| | 8.44 (m, 1 H), 8.15 (s, 2 H), 8.07-8.06 (m, 1 H), 7.68-7.64 | [M + H]⁺ | |
| | (m, 2 H), 7.45-7.43 (m, 1 H), 7.42-7.38 (m, 1 H), 7.33- | (Rt = 1.873 | |
| | 7.31 (m, 1 H), 4.59 (br s, 1 H), 4.37-4.33 (m, 1 H), 3.23- | min) | |
| | 3.21 (m, 1 H), 3.11-3.07 (m, 1 H), 2.97-2.90 (m, 1 H), | | |
| | 2.44 (br s, 3 H), 2.26 (br s, 3 H) | | |
| 131 | (400 MHz, CDCl₃) δ 9.19 (s, 1 H), 8.68-8.67 (m, 1 H), | m/z 533.2 | 100% |
| | 8.56 (br s, 1 H), 8.46-8.45 (m, 1 H), 8.16-8.15 (m, 1 H), | [M + H]⁺ | |
| | 8.15 (s, 1 H), 8.07-8.06 (m, 1 H), 7.74-7.69 (m, 1 H), | (Rt = 1.676 | |
| | 7.59-7.56 (m, 1 H), 7.45-7.43 (m, 1 H), 5.07 (br s, 1 H), | min) | |
| | 4.38-4.35 (m, 1 H), 3.24-3.20 (m, 2 H), 2.92 (br s, 1 H), | | |
| | 2.58-2.55 (m, 1 H), 2.44 (br s, 3 H), 2.40-2.35 (m, 1 H), | | |
| | 2.25-2.19 (m, 1 H) | | |
| 132 | (400 MHz, CDCl₃) δ 9.19 (s, 1 H), 8.67-8.66 (m, 1 H), | m/z 533.2 | 98% |
| | 8.55 (s, 1 H), 8.46-8.45 (m, 1 H), 8.16-8.15 (m, 1 H), | [M + H]⁺ | |
| | 8.08-8.06 (m, 1 H), 7.75-7.68 (m, 1 H), 7.58-7.56 (m, 1 | (Rt = 1.960 | |
| | H), 7.45-7.43 (m, 1 H), 5.09-5.03 (m, 1 H), 4.37-4.32 (m, | min) | |
| | 1 H), 3.22-3.19 (m, 2 H), 2.92-2.87 (m, 1 H), 2.58-2.55 | | |
| | (m, 1 H), 2.42 (s, 3 H), 2.38-2.32 (m, 1 H), 2.24-2.19 (m, | | |
| | 1 H) | | |
| 133 | (400 MHz, CDCl₃) δ 9.16 (s, 1 H), 8.54-8.51 (m, 2 H), | m/z 516.2 | 80% |
| | 8.15 (s, 1 H), 8.11-8.10 (m, 1 H), 7.88-7.86 (m, 1 H), | [M + H]⁺ | |
| | 7.68-7.61 (m, 2 H), 7.39-7.37 (m, 1 H), 7.31-7.29 (m, 1 | (Rt = 1.676 | |
| | H), 7.17-7.15 (m, 1 H), 4.57 (br s, 1 H), 4.35 (br s, 1 H), | min) | |
| | 3.22-3.20 (m, 1 H), 3.10-3.06 (m, 1 H), 2.91 (br s, 1 H), | | |
| | 2.49-2.45 (m, 2 H), 2.42 (br s, 3 H), 2.24 (br s, 1 H) | | |
| 134 | (400 MHz, CDCl₃) δ 9.16 (s, 1 H), 8.55-8.53 (m, 2 H), | m/z 516.2 | 77% |
| | 8.16 (s, 1 H), 8.11-8.09 (m, 1 H), 7.89-7.86 (m, 1 H), | [M + H]⁺ | |
| | 7.66-7.62 (m, 2 H), 7.39-7.37 (m, 1 H), 7.31-7.29 (m, 1 | (Rt = 2.289 | |
| | H), 7.17-7.16 (m, 1 H), 4.58 (br s, 1 H), 4.37-4.34 (m, 1 | min) | |
| | H), 3.23-3.20 (m, 1 H), 3.08 (br s, 1 H), 2.92 (br s, 1 H), | | |
| | 2.46-2.44 (m, 2 H), 2.43 (br s, 3 H), 2.24-2.22 (m, 1 H) | | |
| 135 | (400 MHz, CDCl₃) δ 9.17-9.16 (m, 1 H), 8.56 (br s, 1 H), | m/z 561.2 | 99% |
| | 8.14 (s, 1 H), 8.13-8.12 (m, 1 H), 7.82 (br s, 1 H), 7.67- | [M + H]⁺ | |
| | 7.60 (m, 2 H), 7.40-7.37 (m, 2 H), 7.31-7.28 (m, 1 H), | (Rt = 2.062 | |
| | 6.93-6.90 (m, 1 H), 4.59 (br s, 1 H), 4.37 (br s, 1 H), 3.99 | min) | |
| | (s, 3 H), 3.25-3.23 (m, 1 H), 3.10 (br s, 1 H), 3.00-2.94 | | |
| | (m, 1 H), 2.51-2.46 (m, 1 H), 2.44 (br s, 3 H), 2.30-2.23 | | |
| | (m, 2 H) | | |
| 136 | (400 MHz, CDCl₃) δ 9.16 (s, 1 H), 8.54 (br s, 1 H), 8.13- | m/z 561.2 | 98% |
| | 8.12 (m, 2 H), 7.81-7.79 (m, 1 H), 7.67-7.60 (m, 2 H), | [M + H]⁺ | |
| | 7.41-7.37 (m, 2 H), 7.33-7.31 (m, 1 H), 6.93-6.90 (m, 1 | (Rt = 1.779 | |
| | H), 4.57-4.52 (m, 1 H), 4.36-4.32 (m, 1 H), 3.99 (s, 3 H), | min) | |
| | 3.24-3.21 m, 1 H), 3.10-3.06 (m, 1 H), 2.94-2.89 (m, 1 H), | | |
| | 2.51-2.47 (m, 1 H), 2.42 (s, 3 H), 2.28-2.20 (m, 2 H) | | |
| 137 | (400 MHz, CD₃OD) δ 9.17 (s, 1 H), 8.75-8.71 (m, 1 H), | m/z 562.2 | 98% |
| | 8.71 (s, 1 H), 8.57-8.55 (m, 1 H), 8.40 (br s, 1 H), 8.00- | [M + H]⁺ | |
| | 7.98 (m, 1 H), 7.79-7.74 (m, 2 H), 7.49-7.45 (m, 1 H), | (Rt = 4.930 | |
| | 7.16-7.13 (m, 1 H), 3.94 (s, 3 H), 3.84-3.80 (m, 1 H), | min) | |
| | 3.70-3.66 (m, 2 H), 3.28-3.24 (m, 3 H), 3.04-2.98 (m, 1 | | |
| | H), 2.94 (s, 3 H), 2.82-2.76 (m, 1 H), 2.59-2.56 (m, 1 H) | | |
| 138 | (400 MHz, CD₃OD) δ 9.19 (s, 1 H), 8.76-8.73 (m, 2 H), | m/z 562.2 | 91% |
| | 8.61-8.59 (m, 1 H), 8.46 (br s, 1 H), 8.01-7.96 (m, 1 H), | [M + H]⁺ | |
| | 7.80-7.77 (m, 2 H), 7.51-7.48 (m, 1 H), 7.16-7.14 (m, 1 | (Rt = 5.823 | |
| | H), 3.99-3.96 (m, 1 H), 3.94 (s, 3 H), 3.86-3.84 (m, 1 H), | min) | |
| | 3.28-3.21 (m, 3 H), 3.09 (s, 3 H), 2.88-2.85 (m, 1 H), | | |
| | 2.64-2.61 (m, 1 H) | | |
| 139 | (400 MHz, CDCl₃) & 9.16 (s, 1 H), 8.55-8.50 (m, 2 H), | m/z 564.3 | 100% |
| | 8.17 (s, 1 H), 8.11 (br s, 1 H), 7.87-7.85 (m, 1 H), 7.66- | [M + H]⁺ | |
| | 7.60 (m, 2 H), 7.44-7.42 (m, 1 H), 7.34-7.32 (m, 1 H), | (Rt = 4.322 | |
| | 7.18-7.14 (m, 1 H), 6.77-6.43 (m, 1 H), 4.55-4.37 (m, 2 | min) | |
| | H), 3.35-3.20 (m, 2 H), 2.99-2.85 (m, 2 H), 2.45 (br s, 3 | | |
| | H), 2.23-1.93 (m, 2 H) | | |
| 140 | (400 MHz, CDCl₃) ¿ 9.16 (s, 1 H), 8.54-8.50 (m, 2 H), | m/z 564.3 | 100% |
| | 8.17 (s, 1 H), 8.11 (br s, 1 H), 7.87-7.85 (m, 1 H), 7.66- | [M + H]⁺ | |
| | 7.60 (m, 2 H), 7.44-7.42 (m, 1 H), 7.34-7.32 (m, 1 H), | (Rt = 3.878 | |
| | 7.18-7.14 (m, 1 H), 6.76-6.43 (m, 1 H), 4.55-4.37 (m, 2 | min) | |
| | H), 3.35-3.20 (m, 2 H), 2.99-2.85 (m, 2 H), 2.45 (br s, 3 | | |
| | H), 2.23-1.92 (m, 2 H) | | |
| 141 | (400 MHz, CDCl₃) & 9.16 (s, 1 H), 8.56 (br s, 1 H), 8.45- | m/z 580.2 | 100% |
| | 8.44 (m, 1 H), 8.16 (s, 1 H), 8.15 (br s, 1 H), 8.08 (br s, 1 | [M + H]⁺ | |
| | H), 7.66-7.60 (m, 2 H), 7.44-7.41 (m, 2 H), 7.35-7.33 (m, | (Rt = 3.787 | |
| | 1 H), 6.83-6.40 (m, 1 H), 4.57-4.49 (m, 1 H), 4.39-4.33 | min) | |
| | (m, 1 H), 3.29-3.22 (m, 2 H), 3.00-2.80 (m, 2 H), 2.45 (br | | |
| | s, 3 H), 2.32-2.19 (m, 2 H) | | |
| 142 | (400 MHz, CDCl₃) δ 9.15 (s, 1 H), 8.59 (br s, 1 H), 8.45- | m/z 580.2 | 100% |
| | 8.44 (m, 1 H), 8.16 (s, 1 H), 8.15 (br s, 1 H), 8.08 (br s, 1 | [M + H]⁺ | |
| | H), 7.66-7.60 (m, 2 H), 7.44-7.41 (m, 2 H), 7.35-7.33 (m, | (Rt = 3.009 | |
| | 1 H), 6.83-6.40 (m, 1 H), 4.57-4.49 (m, 1 H), 4.39-4.33 | min) | |
| | (m, 1 H), 3.29-3.22 (m, 2 H), 3.00-2.80 (m, 2 H), 2.45 (br | | |
| | s, 3 H), 2.32-2.19 (m, 2 H) | | |
| 143 | (400 MHz, CD₃OD) δ 9.14 (s, 1 H), 8.70 (s, 1 H), 8.61 (br | m/z 547.2 | N/A |
| | s, 1 H), 7.91-7.61 (m, 4 H), 7.61-7.41 (m, 2 H), 7.37-7.32 | [M + H]⁺ | |
| | (m, 1 H), 6.97-6.95 (m, 1 H), 4.65-4.51 (m, 1 H), 4.37- | | |
| | 4.27 (m, 1 H), 3.24-3.08 (m, 2 H), 2.71-2.35 (m, 7 H) | | |
| 144 | (400 MHz, CD₃OD) δ 9.11 (s, 1 H), 8.64 (s, 1 H), 8.29 (br | m/z 549.2 | 100% |
| | s, 1 H), 7.76-7.72 (m, 2 H), 7.64-7.62 (m, 1 H), 7.52-7.46 | [M + H]⁺ | |
| | (m, 3 H), 7.22-7.20 (m, 1 H), 4.55-4.49 (m, 1 H), 4.24- | (Rt = 1.466 | |
| | 4.18 (m, 1 H), 3.17-3.09 (m, 2 H), 3.01-2.95 (m, 1 H), | min) | |
| | 2.47-2.38 (m, 5 H), 2.29-2.23 (m, 1 H) | | |
| 145 | (400 MHz, CD₃OD) δ 9.11 (s, 1 H), 8.64 (s, 1 H), 8.30 (br | m/z 549.2 | 100% |
| | s, 1 H), 7.79-7.71 (m, 2 H), 7.64-7.62 (m, 1 H), 7.54-7.46 | [M + H]⁺ | |
| | (m, 3 H), 7.22-7.20 (m, 1 H), 4.55-4.49 (m, 1 H), 4.27- | (Rt = 1.676 | |
| | 4.15 (m, 1 H), 3.18-3.08 (m, 2 H), 3.02-2.93 (m, 1 H), | min) | |
| | 2.47-2.38 (m, 5 H), 2.29-2.23 (m, 1 H) | | |
| 146 | (400 MHz, CD₃OD) δ 9.14 (s, 1 H), 8.73-8.72 (m, 1 H), | m/z 534.2 | N/A |
| | 8.67 (s, 1 H), 8.37 (br s, 1 H), 7.98-7.93 (m, 1 H), 7.77- | [M + H]⁺ | |
| | 7.75 (m, 1 H), 7.40-7.38 (m, 1 H), 7.26-7.19 (m, 2 H), | | |
| | 5.06-5.00 (m, 1 H), 4.30-4.23 (m, 1 H), 3.25-3.16 (m, 2 | | |
| | H), 3.01-2.98 (m, 1 H), 2.48-2.44 (m, 5 H), 2.28-2.25 (m, | | |
| | 1 H) | | |
| 147 | (400 MHz, CD₃OD) o 9.14 (s, 1 H), 8.73-8.72 (m, 1 H), | m/z 550.2 | N/A |
| | 8.66 (s, 1 H), 8.34 (br s, 1 H), 7.98-7.93 (m, 1 H), 7.77- | [M + H]⁺ | |
| | 7.75 (m, 1 H), 7.65-7.63 (m, 1 H), 7.52-7.50 (m, 1 H), | | |
| | 7.24-7.19 (m, 1 H), 5.07-5.00 (m, 1 H), 4.27-4.22 (m, 1 | | |
| | H), 3.22-3.17 (m, 2 H), 3.01-2.98 (m, 1 H), 2.48-2.43 (m, | | |
| | 5 H), 2.27-2.25 (m, 1 H) | | |
| 148 | (400 MHz, CD₃OD) δ 9.01 (s, 1 H), 8.63-8.49 (m, 2 H), | m/z 530.3 | 100% |
| | 8.15 (br s, 1 H), 7.73-7.59 (m, 3 H), 7.46-7.33 (m, 2 H), | [M + H]⁺ | |
| | 7.32-7.18 (m, 1 H), 4.49-4.42 (m, 1 H), 4.17-4.15 (m, 1 | (Rt = 1.404 | |
| | H), 3.20-3.14 (m, 2 H), 3.01-2.93 (m, 1 H), 2.70-2.45 (m, | min) | |
| | 3 H), 2.42-2.22 (m, 2 H), 1.10-1.01 (m, 3 H) | | |
| 149 | (400 MHz, CD₃OD) δ 9.00 (s, 1 H), 8.59-8.50 (m, 2 H), | m/z 530.3 | 96% |
| | 8.15 (br s, 1 H), 7.73-7.58 (m, 3 H), 7.46-7.32 (m, 2 H), | [M + H]⁺ | |
| | 7.32-7.18 (m, 1 H), 4.43 (br s, 1 H), 4.16 (br s, 1 H), 3.16- | (Rt = 1.618 | |
| | 3.02 (m, 2 H), 2.98-2.88 (m, 1 H), 2.67-2.41 (m, 3 H), | min) | |
| | 2.35-2.20 (m, 2 H), 1.10-0.99 (m, 3 H) | | |
| 150 | (400 MHz, CD₃OD) δ 8.90 (s, 1 H), 8.44 (s, 1 H), 8.15 (br | m/z 591.3 | 100% |
| | s, 1 H), 7.69-7.62 (m, 1 H), 7.60-7.50 (m, 2 H), 7.34-7.31 | [M + H]⁺ | |
| | (m, 3 H), 7.00-6.90 (m, 1 H), 4.40-4.26 (m, 1 H), 4.15- | (Rt = 2.531 | |
| | 3.95 (m, 3 H), 3.86-3.70 (m, 2 H), 3.03-2.84 (m, 2 H), | min) | |
| | 2.78-2.66 (m, 1 H), 2.44-2.28 (m, 1 H), 2.21 (br s, 4H), | | |
| | 2.12-2.04 (m, 1 H) | | |
| 151 | (400 MHz, CD₃OD) δ 8.90 (s, 1 H), 8.43 (s, 1 H), 8.18 (s, | m/z 591.3 | 100% |
| | 1 H), 7.66-7.65 (m, 1 H), 7.60-7.50 (m, 2 H), 7.34-7.20 | [M + H]⁺ | |
| | (m, 3 H), 6.97-6.90 (m, 1 H), 4.42-4.26 (m, 1 H), 4.15- | (Rt = 1.842 | |
| | 3.95 (m, 3 H), 3.85-3.72 (m, 2 H), 2.99-2.85 (m, 2 H), | min) | |
| | 2.79-2.65 (m, 1 H), 2.43-2.29 (m, 1 H), 2.21 (br s, 4 H), | | |
| | 2.09-2.03 (m, 1 H) | | |
| 152 | (400 MHz, CD₃OD) δ 9.11 (s, 1 H), 8.64 (s, 1 H), 8.19 (br | m/z 533.3 | 97% |
| | s, 1 H), 7.80-7.71 (m, 2 H), 7.53-7.43 (m, 2 H), 7.39-7.36 | [M + H]⁺ | |
| | (m, 1 H), 7.25-7.18 (m, 2 H), 4.55-4.49 (m, 1 H), 4.24- | (Rt = 1.344 | |
| | 4.20 (m, 1 H), 3.17-3.09 (m, 2 H), 3.01-2.95 (m, 1 H), | min) | |
| | 2.51- 2.36 (m, 5 H), 2.29-2.23 (m, 1 H) | | |
| 153 | (400 MHz, CD₃OD) δ 9.11 (s, 1 H), 8.64 (s, 1 H), 8.18 (br | m/z 533.2 | 97% |
| | s, 1 H), 7.82-7.68 (m, 2 H), 7.54-7.42 (m, 2 H), 7.40-7.36 | [M + H]⁺ | |
| | (m, 1 H), 7.25-7.18 (m, 2 H), 4.55-4.49 (m, 1 H), 4.24- | (Rt = 1.533 | |
| | 4.19 (m, 1 H), 3.16-3.10 (m, 2 H), 3.01-2.95 (m, 1 H), | min) | |
| | 2.50- 2.38 (m, 5 H), 2.29-2.24 (m, 1 H) | | |
| 154 | (400 MHz, CDCl₃) δ 9.02 (s, 1 H), 8.24 (s, 1 H), 7.93 (s, 2 | m/z 575.3 | 100% |
| | H), 7.76-7.69 (m, 1 H), 7.61-7.53 (m, 1 H), 7.48-7.46 (m, | [M + H]⁺ | |
| | 1 H), 7.22-7.05 (m, 4 H), 6.81-6.71 (m, 1 H), 4.64-4.58 | (Rt = 2.012 | |
| | (m, 1 H), 4.28 (br s, 4 H), 3.88-3.86 (m, 2 H), 3.20-3.18 | min) | |
| | (m, 1 H), 3.05 (br s, 1 H), 2.85-2.79 (m, 1 H), 2.66 (br s, 1 | | |
| | H), 2.39 (s, 3 H), 2.23-2.06 (m, 2 H) | | |
| 155 | (400 MHz, CDCl₃) δ 9.03 (s, 1 H), 8.25 (s, 1 H), 7.94 (s, 2 | m/z 575.3 | 98% |
| | H), 7.77-7.69 (m, 1 H), 7.62-7.55 (m, 1 H), 7.48-7.45 (m, | [M + H]⁺ | |
| | 1 H), 7.22-7.06 (m, 4 H), 6.84-6.71 (m, 1 H), 4.65-4.59 | (Rt = 3.273 | |
| | (m, 1 H), 4.34-4.25 (m, 3 H), 3.90-3.85 (m, 2 H), 3.20- | min) | |
| | 3.18 (m, 1 H), 3.06 (br s, 1 H), 2.85-2.78 (m, 1 H), 2.68 | | |
| | (br s, 1 H), 2.40 (s, 3 H), 2.23-2.09 (m, 2 H) | | |
| 156 | (400 MHz, CDCl₃) δ 9.09 (br s, 1 H), 8.53-8.27 (m, 2 H), | m/z 574.3 | 100% |
| | 8.16 (br s, 1 H), 8.08-7.88 (m, 2 H), 7.64-7.41 (m, 3 H), | [M + H]⁺ | |
| | 7.16 (br s, 2 H), 4.66 (br s, 1 H), 4.29 (br s, 3 H), 3.87 (br | (Rt = 1.967 | |
| | s, 2 H), 3.26-2.95 (m, 2 H), 2.85 (br s, 1 H), 2.40 (br s, 3 | min) | |
| | H), 2.29-2.11 (m, 2 H) | | |
| 157 | (400 MHz, CDCl₃) δ 9.08 (br s, 1 H), 8.45-8.43 (m, 1 H), | m/z 574.3 | 99% |
| | 8.33 (br s, 1 H), 8.16 (br s, 1 H), 8.08-7.88 (m, 2 H), 7.64- | [M + H]⁺ | |
| | 7.43 (m, 3 H), 7.21-7.11 (m, 2 H), 4.65 (br s, 1 H), 4.29 | (Rt = 2.244 | |
| | (br s, 3 H), 3.88 (br s, 2 H), 3.26-2.95 (m, 2 H), 2.85 (br s, | min) | |
| | 1 H), 2.70-2.67 (m, 1 H), 2.41 (s, 3 H), 2.32-2.13 (m, 2 H) | | |
| 158 | (400 MHz, CD₃OD) δ 9.05 (s, 1 H), 8.64 (s, 1 H), 8.16 (s, | m/z 575.3 | 99% |
| | 1 H), 7.72-7.64 (m, 1 H), 7.60-7.52 (m, 1 H), 7.46 (s, 1 | [M + H]⁺ | |
| | H), 7.45-7.37 (m, 1 H), 7.36-7.32 (m, 1 H), 7.30-7.20 (m, | (Rt = 1.461 | |
| | 3 H), 4.59-4.43 (m, 1 H), 4.39-4.04 (m, 3 H), 3.80 (s, 2 | min) | |
| | H), 3.20-3.10 (m, 1 H), 3.03-2.72 (m, 2 H), 2.57-2.22 (m, | | |
| | 6 H) | | |
| 159 | (400 MHz, CD₃OD) δ 9.06 (s, 1 H), 8.64 (s, 1 H), 8.16 (s, | m/z 575.3 | 100% |
| | 1 H), 7.70-7.66 (m, 1 H), 7.57-7.55 (m, 1 H), 7.47 (s, 1 | [M + H]⁺ | |
| | H), 7.44-7.37 (m, 1 H), 7.36-7.32 (m, 1H), 7.31-7.20 (m, 3 | (Rt = 1.656 | |
| | H), 4.59-4.43 (m, 1 H), 4.39-4.07 (m, 3 H), 3.80 (s, 2 H), | min) | |
| | 3.19-3.08 (m, 1 H), 3.07-2.67 (m, 2 H), 2.66-2.33 (m, 4 | | |
| | H), 2.31-2.22 (m, 2 H) | | |
| 160 | (400 MHz, CD₃OD) δ 9.05 (s, 1 H), 8.70 (s, 1 H), 8.58 (s, | m/z 574.3 | 99% |
| | 1 H), 8.17 (s, 1 H), 8.09 (s, 1 H), 7.71-7.61 (m, 2 H), 7.59- | [M + H]⁺ | |
| | 7.52 (m, 1 H), 7.49 (s, 1H), 7.37-7.32 (m, 1 H), 7.26-7.21 | (Rt = 1.863 | |
| | (m, 1 H), 4.61-4.46 (m, 1 H), 4.41-4.04 (m, 3 H), 3.78 (s, | min) | |
| | 2 H), 3.14-3.04 (m, 1 H), 3.03-2.67 (m, 2 H), 2.66-2.13 | | |
| | (m, 6 H) | | |
| 161 | (400 MHz, CD₃OD) δ 9.04 (s, 1 H), 8.69 (s, 1 H), 8.57 (s, | m/z 574.2 | 99% |
| | 1 H), 8.17 (s, 1 H), 8.08 (s, 1 H), 7.72-7.60 (m, 2 H), 7.57- | [M + H]⁺ | |
| | 7.51 (m, 1 H), 7.48 (s, 1H), 7.36-7.30 (m, 1 H), 7.25-7.19 | (Rt = 2.205 | |
| | (m, 1 H), 4.59-4.43 (m, 1 H), 4.38-4.06 (m, 3 H), 3.77 (s, | min) | |
| | 2 H), 3.11-3.04 (m, 1 H), 3.01-2.66 (m, 2 H), 2.65-2.20 | | |
| | (m, 6 H) | | |
| 162 | (400 MHz, CD₃OD) δ 9.14 (br s, 3 H), 8.52 (br s, 1 H), | m/z 569.3 | 94% |
| | 7.71 (br s, 1 H), 7.57-7.56 (m, 1 H), 7.45-7.43 (br d, 1 H), | [M + H]⁺ | |
| | 7.23-7.21 (m, 2 H), 7.13-7.11 (m, 1 H), 4.41 (br s, 1 H), | (Rt = 1.958 | |
| | 4.13 (br s, 4 H), 3.66 (br s, 3 H), 3.00-2.96 (m, 1 H), 2.81 | min) | |
| | (br s, 1 H), 2.57 (br s, 1 H), 2.38-2.21 (m, 4 H) | | |
| 163 | (400 MHz, CD₃OD) δ 9.11 (br s, 3 H), 8.52 (br s, 1 H), | m/z 569.3 | 99% |
| | 7.71-7.68 (m, 1 H), 7.57-7.56 (m, 1 H), 7.44-7.43 (m, 1 | [M + H]⁺ | |
| | H), 7.22-7.21 (m, 2 H), 7.13-7.11 (m, 1 H), 4.42 (br s, 1 | (Rt = 2.301 | |
| | H), 4.12 (br s, 3 H), 3.67 (br s, 2 H), 2.99-2.96 (m, 1 H), | min) | |
| | 2.80 (br s, 1 H), 2.57 (br s, 1 H), 2.39-2.20 (m, 4 H) | | |
| 164 | (400 MHz, CD₃OD) δ 9.17-8.98 (m, 3 H), 8.57 (br s, 1 H), | m/z 585.3 | 74% |
| | 8.09 (br s, 1 H), 7.73-7.53 (m, 4 H), 7.37-7.30 (m, 1 H), | [M + H]⁺ | |
| | 7.23-7.21 (t, 1 H), 4.55 (br s, 1 H), 4.25 (br s, 3 H), 3.79 | (Rt = 4.945 | |
| | (br s, 2 H), 3.13-3.10 (m, 1 H), 2.94 (br s, 1 H), 2.71-2.69 | min) | |
| | (m, 2 H), 2.53-2.33 (m, 4 H) | | |
| 165 | (400 MHz, CD₃OD) δ 9.14 (br s, 3 H), 8.55 (br s, 1 H), | m/z 585.3 | 20% |
| | 8.07 (br s, 1 H), 7.72-7.49 (m, 4 H), 7.35-7.29 (m, 1 H), | [M + H]⁺ | |
| | 7.22-7.20 (m, 1 H), 4.52 (br s, 1 H), 4.24 (br s, 3 H), 3.78 | (Rt = 5.449 | |
| | (br s, 2 H), 2.97 (br s, 1 H), 2.71-2.68 (m, 2 H), 2.50-2.30 | min) | |
| | (m, 4 H) | | |
| 166 | (400 MHz, CD₃OD) δ 9.09 (br s, 1 H), 8.73 (br s, 1 H), | 558.3 | 94% |
| | 8.66-8.64 (m, 1 H), 7.86-7.74 (m, 4 H), 7.49-7.38 (m, 3 | [M + H]⁺; | |
| | H), 4.85 (br s, 1 H), 4.43-4.15 (m, 3 H), 3.53-3.34 (m, 2 | Rt = 4.523 min | |
| | H), 2.94-2.88 (m, 1 H), 2.46-2.43 (m, 1 H), 2.34 (s, 3 H), | | |
| | 2.20-2.09 (m, 3 H) | | |
| 167 | (400 MHz, CD₃OD) δ 9.09 (br s, 1 H), 8.73 (br s, 1 H), | 558.3 | 100% |
| | 8.65-8.63 (m, 1 H), 7.87-7.84 (m, 4 H), 7.51-7.38 (m, 3 | [M + H]⁺; | |
| | H), 4.85 (br s, 1 H), 4.49-4.19 (m, 3 H), 3.53-3.31 (m, 2 | Rt = 3.862 min | |
| | H), 2.95-2.88 (m, 1 H), 2.46-2.42 (m, 1 H), 2.33 (s, 3 H), | | |
| | 2.19-2.08 (m, 3 H) | | |
| 168 | (400 MHz, DMSO-d₆) δ 14.57 (s, 1H), 9.13 (s, 1H), 8.58 | m/z 527.9 | >99%* |
| | (s, 1H), 8.38-8.12 (m, 1H), 7.81-7.71 (m, 2H), 7.67-7.61 | [M + H]⁺ | |
| | (m, 3H), 7.58-7.46 (m, 2H), 4.60-4.46 (m, 2H), 4.45-4.33 | | |
| | (m, 1H), 4.24-4.12 (m, 1H), 2.50-2.41 (m, 1H), 2.29-2.07 | | |
| | (m, 2H), 2.07-1.91 (m, 2H), 1.84-1.74 (m, 2H), 1.63-1.45 | | |
| | (m, 1H) | | |
| 169 | (400 MHz, DMSO-d₆) δ 14.73 (s, 1H), 9.11 (s, 1H), 8.64 | m/z 512.5 | >99%* |
| | (s, 1H), 8.19 (s, 1H), 7.80-7.67 (m, 2H), 7.67-7.58 (m, | [M + H]⁺ | |
| | 1H), 7.56-7.45 (m, 2H), 7.44-7.36 (m, 2H), 4.59-4.43 (m, | | |
| | 2H), 4.42-4.24 (m, 1H), 4.24-4.06 (m, 1H), 2.61-2.52 (m, | | |
| | 1H), 2.29-2.17 (m, 1H), 2.16-2.04 (m, 1H), 2.02-1.92 (m, | | |
| | 2H), 1.85-1.72 (m, 2H), 1.59-1.47 (m, 1H) | | |
| 170 | (400 MHz, DMSO-d₆) δ 14.62 (s, 1H), 9.17 (s, 1H), 8.72 | m/z 513.4 | >99%* |
| | (d, 1H), 8.61 (s, 1H), 8.21 (s, 1H), 8.09 (t, 1H), 7.85-7.77 | [M + H]⁺ | |
| | (m, 1H), 7.68-7.60 (m, 1H), 7.48-7.39 (m, 2H), 4.91-4.77 | | |
| | (m, 1H), 4.59-4.42 (m, 2H), 4.27-4.14 (m, 1H), 2.35-1.89 | | |
| | (m, 5H), 1.84-1.71 (m, 2H), 1.65-1.46 (m, 1H) | | |
| 171 | (400 MHz, DMSO-d₆) δ 14.42 (s, 1H), 9.18 (s, 1H), 8.61 | m/z 519.3 | >99%* |
| | (s, 1H), 8.24 (s, 1H), 8.17 (d, 1H), 8.04-7.95 (m, 1H), | [M + H]⁺ | |
| | 7.94-7.84 (m, 2H), 7.69-7.60 (m, 1H), 7.49-7.35 (m, 2H), | | |
| | 4.57-4.46 (m, 2H), 4.46-4.38 (m, 1H), 4.26-4.06 (m, 1H), | | |
| | 2.47-2.42 (m, 1H), 2.24-2.13 (m, 2H), 2.13-2.03 (m, 1H), | | |
| | 2.00-1.88 (m, 1H), 1.83-1.68 (m, 2H), 1.63-1.43 (m, 1H) | | |
| 172 | (400 MHz, DMSO-d₆) δ 14.58 (s, 1H), 9.06 (s, 1H), 8.51 | m/z 570.5 | >99%* |
| | (s, 1H), 8.22-8.06 (m, 1H), 7.69-7.57 (m, 4H), 7.48 (d, | [M + H]⁺ | |
| | 1H), 7.33 (d, 1H), 7.16 (t, 1H), 4.85-4.41 (m, 3H), 4.32- | | |
| | 4.01 (m, 4H), 3.68-3.46 (m, 2H), 2.34-1.71 (m, 7H), 1.64- | | |
| | 1.35 (m, 1H) | | |
| 173 | (400 MHz, DMSO-d₆) δ 14.61 (s, 1H), 9.07 (s, 1H), 8.55 | m/z 554.5 | >99%* |
| | (s, 1H), 8.19 (s, 1H), 7.63 (t, 2H), 7.52-7.38 (m, 3H), 7.34 | [M + H]⁺ | |
| | (d, 1H), 7.16 (t, 1H), 4.92-4.68 (m, 1H), 4.61-4.44 (m, | | |
| | 2H), 4.31-4.02 (m, 4H), 3.70-3.49 (m, 2H), 2.44-2.11 (m, | | |
| | 3H), 2.01-1.91 (m, 1H), 1.85-1.68 (m, 3H), 1.56-1.43 (m, | | |
| | 1H) | | |
| 174 | (400 MHz, DMSO-d₆) δ 14.63 (s, 1H), 9.13 (s, 1H), 8.57 | m/z | >99%* |
| | (s, 1H), 8.27-8.10 (m, 1H), 7.79-7.71 (m, 2H), 7.65 (d, | 494.4[M + H]⁺ | |
| | 1H), 7.61-7.45 (m, 5H), 4.59-4.45 (m, 2H), 4.45-4.31 (m, | | |
| | 1H), 4.21-4.11 (m, 1H), 2.48-2.41 (m, 1H), 2.29-2.06 (m, | | |
| | 2H), 2.05-1.92 (m, 2H), 1.82-1.71 (m, 2H), 1.64-1.47 (m, | | |
| | 1H) | | |
| 175 | (400 MHz, DMSO-d₆) δ 14.62 (s, 1H), 9.13 (s, 1H), 8.57 | m/z 512.3 | >99%* |
| | (s, 1H), 8.32-7.99 (m, 1H), 7.80-7.71 (m, 2H), 7.64-7.46 | [M + H]⁺ | |
| | (m, 3H), 7.41 (d, 1H), 7.27-7.16 (m, 1H), 4.66-4.46 (m, | | |
| | 2H), 4.45-4.30 (m, 1H), 4.20-4.08 (m, 1H), 2.49-2.37 (m, | | |
| | 1H), 2.30-2.13 (m, 1H), 2.13-2.06 (m, 1H), 2.05-1.91 (m, | | |
| | 2H), 1.82-1.71 (m, 2H), 1.62-1.48 (m, 1H) | | |
| 176 | (400 MHz, DMSO-d₆) δ 14.60 (s, 1H), 9.13 (s, 1H), 8.57 | m/z 512.5 | >99%* |
| | (s, 1H), 8.24-8.06 (m, 1H), 7.81-7.65 (m, 3H), 7.55-7.45 | [M + H]⁺ | |
| | (m, 3H), 7.32-7.26 (m, 1H), 4.61-4.44 (m, 2H), 4.44-4.31 | | |
| | (m, 1H), 4.19-4.07 (m, 1H), 2.29-2.06 (m, 3H), 2.03-1.90 | | |
| | (m, 2H), 1.82-1.72 (m, 2H), 1.65-1.48 (m, 1H) | | |
| 177 | (400 MHz, DMSO-d₆) δ 14.07 (s, 1H), 9.13 (s, 1H), 8.60 | m/z 512.5 | >99%* |
| | (s, 1H), 8.33-8.15 (m, 1H), 7.79-7.70 (m, 2H), 7.58-7.39 | [M + H]⁺ | |
| | (m, 5H), 4.73-4.56 (m, 2H), 4.42-4.30 (m, 1H), 4.22-4.09 | | |
| | (m, 1H), 2.60-2.52 (m, 1H), 2.29-2.15 (m, 1H), 2.12-2.04 | | |
| | (m, 1H), 2.04-1.91 (m, 2H), 1.87-1.74 (m, 2H), 1.63-1.48 | | |
| | (m, 1H) | | |
| 178 | Data provided above | | |
| 179 | (400 MHz, DMSO-d₆) δ 14.66 (s, 1H), 10.73 (s, 1H), 9.09 | m/z 528.4 | >99%* |
| | (s, 1H), 8.55 (s, 1H), 8.20 (s, 1H), 7.69-7.60 (m, 1H), | [M + H]⁺ | |
| | 7.54-7.40 (m, 3H), 6.98-6.86 (m, 2H), 4.57-4.44 (m, 2H), | | |
| | 4.39-4.28 (m, 1H), 4.24-4.12 (m, 1H), 2.48-2.39 (m, 1H), | | |
| | 2.25-1.93 (m, 4H), 1.83-1.70 (m, 2H), 1.61-1.49 (m, 1H) | | |
| 180 | (400 MHz, DMSO-d₆) δ 14.62 (s, 1H), 9.11 (s, 1H), 8.56 | m/z 560.5 | >99%* |
| | (s, 1H), 8.17 (s, 1H), 7.81-7.75 (m, 1H), 7.73-7.68 (m, | [M + H]⁺ | |
| | 1H), 7.67-7.61 (m, 1H), 7.54-7.39 (m, 5H), 4.57-4.42 (m, | | |
| | 2H), 4.36-4.24 (m, 1H), 4.16-4.08 (m, 1H), 2.32-2.19 (m, | | |
| | 1H), 2.11-1.95 (m, 3H), 1.85-1.73 (m, 3H), 1.61-1.41 (m, | | |
| | 1H) | | |
| 181 | (400 MHz, DMSO-d₆) δ 9.03 (s, 1H), 8.50 (s, 1H), 8.00 (s, | m/z 568.6 | >99%* |
| | 1H), 7.71-7.55 (m, 2H), 7.53-7.36 (m, 3H), 7.33 (d, 1H), | [M + H]⁺ | |
| | 7.15 (t, 1H), 4.85-4.58 (m, 1H), 4.59-4.36 (m, 2H), 4.31- | | |
| | 3.91 (m, 4H), 3.69-3.43 (m, 2H), 2.41-2.18 (m, 5H), 2.13- | | |
| | 0.99 (m, 6H) | | |
| 183 | (400 MHz, DMSO-d₆) δ 14.54 (s, 1H), 9.13 (s, 1H), 8.55 | m/z 544.5 | >99% |
| | (s, 1H), 8.22 (s, 1H), 7.76-7.69 (m, 2H), 7.65-7.60 (m, | [M + H]⁺ | |
| | 3H), 7.54-7.45 (m, 2H), 5.25 (d, 1H), 4.57-4.33 (m, 3H), | | |
| | 4.13-4.03 (m, 1H), 3.94-3.83 (m, 1H), 2.47-2.40 (m, 1H), | | |
| | 2.24-2.16 (m, 1H), 2.11-1.99 (m, 1H), 1.97-1.88 (m, 1H), | | |
| | 1.78-1.69 (m, 1H), 1.59-1.46 (m, 1H) | | |
| 184 | (400 MHz, DMSO-d₆) δ 14.70 (s, 1H), 9.13 (s, 1H), 8.57 | m/z 528.5 | >99% |
| | (s, 1H), 8.25-8.01 (m, 1H), 7.79-7.69 (m, 2H), 7.68-7.60 | [M + H]⁺ | |
| | (m, 1H), 7.54-7.36 (m, 4H), 5.25 (d, 1H), 4.58-4.48 (m, | | |
| | 1H), 4.46-4.33 (m, 2H), 4.16-4.04 (m, 1H), 3.94-3.84 (m, | | |
| | 1H), 2.48-2.42 (m, 1H), 2.23-1.89 (m, 3H), 1.79-1.69 (m, | | |
| | 1H), 1.60-1.47 (m, 1H) | | |
| 185 | (400 MHz, DMSO-d₆) δ 9.01 (s, 1H), 8.46 (s, 1H), 8.22 (s, | m/z 528.2 | >99% |
| | 2H), 7.72 (t, 2H), 7.67-7.61 (m, 1H), 7.56-7.36 (m, 4H), | [M + H]⁺ | |
| | 5.24 (d, 1H), 4.54-4.34 (m, 3H), 4.13-4.03 (m, 1H), 3.92- | | |
| | 3.81 (m, 1H), 2.23-2.13 (m, 1H), 2.12-1.99 (m, 1H), 1.96- | | |
| | 1.86 (m, 1H), 1.76-1.42 (m, 3H) | | |
| 186 | (400 MHz, DMSO-d₆) δ 9.04 (s, 1H), 8.49 (s, 1H), 8.23 (s, | m/z 546.2 | >99% |
| | 2H), 7.85-7.74 (m, 1H), 7.65-7.59 (m, 1H), 7.44-7.38 (m, | [M + H]⁺ | |
| | 4H), 5.23 (d, 1H), 4.52-4.29 (m, 3H), 4.06-3.87 (m, 2H), | | |
| | 2.17-1.96 (m, 3H), 1.96-1.87 (m, 1H), 1.73-1.65 (m, 1H), | | |
| | 1.65-1.50 (m, 1H) | | |
| 187 | (400 MHz, DMSO-d₆) δ 9.18 (s, 1H), 9.12-8.97 (m, 2H), | m/z 557.2 | >99% |
| | 8.88 (s, 1H), 7.88-7.71 (m, 1H), 7.67-7.51 (m, 2H), 7.44- | [M + H]⁺ | |
| | 7.35 (m, 4H), 5.26 (d, 1H), 4.56-4.34 (m, 2H), 4.33-4.26 | | |
| | (m, 1H), 4.08-3.98 (m, 1H), 3.97-3.85 (m, 1H), 2.64-2.52 | | |
| | (m, 1H), 2.23-2.11 (m, 1H), 2.08-1.91 (m, 2H), 1.82-1.70 | | |
| | (m, 1H), 1.67-1.49 (m, 1H) | | |
| 188 | (400 MHz, DMSO-d₆) δ 14.53 (s, 1H), 9.12 (s, 1H), 8.54 | m/z 576.5 | >99% |
| | (s, 1H), 8.22 (s, 1H), 7.72 (t, 1H), 7.62 (d, 3H), 7.47-7.40 | [M + H]⁺ | |
| | (m, 4H), 5.27-5.17 (m, 1H), 4.55-4.36 (m, 2H), 4.34-4.30 | | |
| | (m, 1H), 4.08-3.96 (m, 1H), 3.93-3.85 (m, 1H), 2.25-2.15 | | |
| | (m, 1H), 2.10-1.89 (m, 3H), 1.76-1.70 (m, 1H), 1.60-1.50 | | |
| | (m, 1H) | | |
| 189 | (400 MHz, DMSO-d₆) δ 14.35 (s, 1H), 9.13 (s, 1H), 8.56 | m/z 594.2 | >99% |
| | (s, 1H), 8.31-8.16 (m, 1H), 7.86-7.72 (m, 1H), 7.67-7.59 | [M + H]⁺ | |
| | (m, 1H), 7.50-7.27 (m, 5H), 5.28-5.12 (m, 1H), 4.58-4.26 | | |
| | (m, 3H), 4.05-3.84 (m, 2H), 2.59-2.52 (m, 1H), 2.22-1.87 | | |
| | (m, 3H), 1.80-1.70 (m, 1H), 1.67-1.50 (m, 1H) | | |
| 190 | (400 MHz, DMSO-d₆) δ 9.12 (s, 1H), 8.65 (d, 1H), 8.48 | m/z 556.5 | >99% |
| | (s, 1H), 7.95-7.85 (m, 1H), 7.77-7.68 (m, 2H), 7.65-7.57 | [M + H]⁺ | |
| | (m, 2H), 7.54-7.44 (m, 2H), 7.43-7.34 (m, 2H), 5.23 (d, | | |
| | 1H), 4.52-4.31 (m, 3H), 4.13-4.02 (m, 1H), 3.93-3.83 (m, | | |
| | 1H), 2.70-2.52 (m, 1H), 2.26-2.12 (m, 1H), 2.07-1.85 (m, | | |
| | 2H), 1.73-1.61 (m, 1H), 1.61-1.45 (m, 1H) | | |
| 191 | (400 MHz, DMSO-d₆) δ 9.14 (s, 1H), 8.64 (d, 1H), 8.50 | m/z 574.3 | >99% |
| | (s, 1H), 7.95-7.86 (m, 1H), 7.85-7.72 (m, 1H), 7.65-7.55 | [M + H]⁺ | |
| | (m, 2H), 7.44-7.34 (m, 4H), 5.23 (d, 1H), 4.52-4.24 (m, | | |
| | 3H), 4.04-3.96 (m, 1H), 3.95-3.87 (m, 1H), 2.62-2.51 (m, | | |
| | 1H), 2.16-2.07 (m, 1H), 2.05-1.86 (m, 2H), 1.72-1.63 (m, | | |
| | 1H), 1.62-1.46 (m, 1H) | | |
| 192 | (400 MHz, DMSO-d₆) δ 14.58 (s, 1H), 10.77 (s, 1H), 9.10 | m/z 558.3 | >99% |
| | (s, 1H), 8.58 (s, 1H), 8.27-8.15 (m, 1H), 7.69-7.62 (m, | [M + H]⁺ | |
| | 1H), 7.55-7.47 (m, 1H), 7.47-7.40 (m, 2H), 6.96-6.81 (m, | | |
| | 2H), 4.56-4.33 (m, 4H), 3.94-3.83 (m, 1H), 3.20 (s, 3H), | | |
| | 2.68-2.56 (m, 1H), 2.42-2.32 (m, 1H), 2.30-1.91 (m, 4H) | | |
| 193 | (400 MHz, CD₃OD) δ 9.06 (s, 1H), 8.71 (s, 1H), 7.87 (s, | m/z 542.2 | >99% |
| | 1H), 7.78-7.69 (m, 2H), 7.59-7.54 (m, 1H), 7.48-7.39 (m, | [M + H]⁺ | |
| | 3H), 7.37-7.29 (m, 1H), 4.63-4.48 (m, 3H), 4.25-4.18 (m, | | |
| | 1H), 4.10-3.99 (m, 1H), 2.74-2.58 (m, 1H), 2.35 (s, 3H), | | |
| | 2.33-2.25 (m, 1H), 2.21-2.02 (m, 2H), 1.98-1.91 (m, 1H), | | |
| | 1.71-1.59 (m, 1H) | | |
| 194 | (400 MHz, CDCl₃) δ 8.84 (d, 2H), 8.76 (s, 1H), 8.45 (d, | m/z 594.5 | >99% |
| | 1H), 7.88 (d, 1H), 7.58-7.51 (m, 1H), 7.48 (d, 1H), 7.45- | [M + H]⁺ | |
| | 7.37 (m, 2H), 7.25-7.12 (m, 4H), 4.88-4.76 (m, 1H), 4.69- | | |
| | 4.58 (m, 1H), 4.40 (s, 2H), 4.26 (s, 2H), 3.92 (s, 1H), | | |
| | 3.86-3.75 (m, 2H), 3.24 (s, 3H), 2.74-2.63 (m, 1H), 2.60- | | |
| | 2.50 (m, 1H), 2.39-2.21 (m, 3H), 1.96-1.88 (m, 2H) | | |
| 195 | (400 MHz, DMSO-d₆) δ 14.47 (s, 1H), 9.14 (s, 1H), 8.60 | m/z 542.1 | >99% |
| | (s, 1H), 8.42-8.17 (m, 1H), 7.81-7.71 (m, 2H), 7.68-7.60 | [M + H]⁺ | |
| | (m, 1H), 7.58-7.44 (m, 4H), 4.62-4.44 (m, 3H), 4.32-4.23 | | |
| | (m, 1H), 3.62-3.53 (m, 1H), 3.27 (s, 3H), 2.45-2.35 (m, | | |
| | 2H), 2.22-2.16 (m, 1H), 2.12-2.04 (m, 2H), 1.79-1.68 (m, | | |
| | 1H) | | |
| 196 | (400 MHz, DMSO-d₆) δ 9.14 (s, 1 H), 8.35 (s, 1 H), 8.24 | m/z 527.3 | N/A |
| | (s, 1 H), 7.75-7.73 (m, 2 H), 7.66-7.64 (m, 1 H), 7.53-7.43 | [M + H]⁺ | |
| | (m, 4 H), 4.49 (br s, 3 H), 4.34-4.30 (m, 1 H), 3.24 (br s, 1 | | |
| | H), 2.43-2.39 (m, 1 H), 2.28-1.99 (m, 4 H), 1.83-1.77 (m, | | |
| | 1 H) | | |
| 197 | (400 MHz, CDCl₃) δ 8.99 (s, 1 H), 8.43 (s, 1 H), 8.03 (br | m/z 569.3 | 100% |
| | s, 1 H), 7.53-7.47 (m, 1 H), 7.44-7.34 (m, 3 H), 7.19-7.15 | [M + H]⁺ | |
| | (m, 1 H), 7.12-7.05 (m, 2 H), 4.67-4.34 (m, 4 H), 4.21 (br | (Rt = 0.730 | |
| | s, 2 H), 3.80 (br s, 2 H), 3.02-2.91 (m, 2 H), 2.79 (br s, 1 | min) | |
| | H), 2.47-2.35 (m, 3 H), 2.30 (s, 3 H) | | |
| 198 | (400 MHz, CDCl₃) δ 8.97 (s, 1 H), 8.42 (s, 1 H), 8.03 (br | m/z 569.3 | 95% |
| | s, 1 H), 7.54-7.46 (m, 1 H), 7.44-7.32 (m, 4 H), 7.18-7.14 | [M + H]⁺ | |
| | (m, 1 H), 7.12-7.05 (m, 2 H), 4.65-4.30 (m, 4 H), 4.21 (br | (Rt = 1.668 | |
| | s, 2 H), 3.80 (br s, 2 H), 3.00-2.93 (m, 2 H), 2.78 (br s, 1 | min) | |
| | H), 2.50-2.34 (m, 3 H), 2.29 (s, 3 H) | | |
| 199 | (400 MHz, CDCl₃) δ 9.15 (br s, 1 H), 8.35 (br s, 1 H), | m/z 555.2 | 100% |
| | 7.98 (br s, 2 H), 7.72-7.65 (m, 2 H), 7.51-7.40 (m, 3 H), | [M + H]⁺ | |
| | 7.35-7.29 (m, 2 H), 5.00-4.90 (m, 1 H), 4.48-4.39 (m, 3 | (Rt = 3.022 | |
| | H), 4.31-4.18 (m, 1 H), 4.14-4.01 (m, 1 H), 3.40-3.30 (m, | min) | |
| | 1 H), 3.11-2.92 (m, 1 H), 2.86-2.77 (m, 1 H), 2.51-2.32 | | |
| | (m, 1 H), 2.18-2.15 (m, 3 H) | | |
| 200 | (400 MHz, CDCl₃) ¿ 9.14 (br s, 1 H), 8.34 (br s, 1 H), | m/z 555.2 | 100% |
| | 7.98 (br s, 2 H), 7.73-7.64 (m, 2 H), 7.53-7.41 (m, 3 H), | [M + H]⁺ | |
| | 7.37-7.29 (m, 2 H), 5.00-4.90 (m, 1 H), 4.48-4.44 (m, 2 | (Rt = 1.996 | |
| | H), 4.40-4.37 (m, 1 H), 4.30-4.18 (m, 1 H), 4.14-4.01 (m, | min) | |
| | 1 H), 3.43-3.30 (m, 1 H), 3.10-2.92 (m, 1 H), 2.86-2.77 | | |
| | (m, 1 H), 2.51-2.29 (m, 1 H), 2.18-2.15 (m, 3 H) | | |
| 201 | (400 MHz, CDCl₃) δ 9.19 (s, 1 H), 8.66 (s, 1 H), 8.16-8.14 | m/z 573.3 | N/A |
| | (m, 1 H), 7.58-7.36 (m, 3 H), 7.18-7.08 (m, 3 H), 4.91- | [M + H]⁺ | |
| | 4.89 (m, 1 H), 4.53-4.20 (m, 5 H), 3.98-3.95 (m, 1 H), | | |
| | 3.42-3.36 (m, 1 H), 3.01-2.82 (m, 1 H), 2.37-2.28 (m, 1 | | |
| | H), 2.10-2.03 (m, 3 H) | | |
| 202 | Data provided above | | |
| 203 | Data provided above | | |
| 204 | (400 MHz, DMSO-d₆) δ 14.67 (s, 1H), 9.13 (s, 1H), 8.66 | m/z 528.4 | >99%* |
| | (s, 1H), 8.12 (s, 1H), 7.81-7.69 (m, 2H), 7.57-7.43 (m, | [M + H]⁺ | |
| | 3H), 7.38 (t, 1H), 7.10 (d, 1H), 5.49-5.33 (m, 2H), 4.37- | | |
| | 4.20 (m, 2H), 2.61-2.54 (m, 1H), 2.35-2.16 (m, 1H), 2.04- | | |
| | 1.85 (m, 4H), 1.71-1.61 (m, 1H), 1.51-1.38 (m, 1H) | | |
| 205 | (400 MHz, DMSO-d₆) δ 9.13 (s, 1H), 8.70 (s, 1H), 8.31 (s, | m/z 592.0 | >99%* |
| | 1H), 7.82-7.65 (m, 3H), 7.59-7.53 (m, 1H), 7.51-7.44 (m, | [M + H]⁺ | |
| | 2H), 7.40-6.94 (m, 2H), 5.50-5.33 (m, 2H), 4.34-4.21 (m, | | |
| | 1H), 4.21-4.09 (m, 1H), 2.37-1.85 (m, 6H), 1.73-1.61 (m, | | |
| | 1H), 1.59-1.38 (m, 1H) | | |
| 206 | (400 MHz, DMSO-d₆) δ 14.14 (s, 1H), 9.13 (s, 1H), 8.66 | m/z 543.5 | >99%* |
| | (s, 1H), 8.43-8.17 (m, 1H), 7.80-7.71 (m, 2H), 7.58-7.45 | [M + H]⁺ | |
| | (m, 3H), 7.28 (d, 1H), 6.97 (s, 1H), 6.77 (d, 1H), 4.66-4.55 | | |
| | (m, 2H), 4.42-4.31 (m, 1H), 4.31-4.20 (m, 1H), 2.49-2.42 | | |
| | (m, 1H), 2.31-2.15 (m, 1H), 2.01-1.84 (m, 4H), 1.66-1.56 | | |
| | (m, 1H), 1.54-1.39 (m, 1H) | | |
| 207 | Data provided above | | |
| 208 | Data provided above | | |
| 209 | Data provided above | | |
| 210 | (400 MHz, DMSO-d₆) δ 14.67 (s, 1H), 9.18 (s, 1H), 8.70 | m/z 535.5 | >99%* |
| | (s, 1H), 8.17 (d, 2H), 8.03-7.94 (m, 1H), 7.93-7.87 (m, | [M + H]⁺ | |
| | 2H), 7.49 (dd, 1H), 7.44-7.32 (m, 1H), 7.11 (dd, 1H), | | |
| | 5.46-5.37 (m, 2H), 4.44-4.21 (m, 2H), 2.27-2.15 (m, 2H), | | |
| | 1.92-1.84 (m, 2H), 1.68-1.62 (m, 1H), 1.50-1.41 (m, 3H) | | |
| 211 | (400 MHz, DMSO-d₆) δ 14.64 (s, 1H), 9.11 (s, 1H), 8.65 | m/z 576.6 | >99%* |
| | (s, 1H), 7.76 (t, 1H), 7.69 (d, 1H), 7.57-7.17 (m, 6H), | [M + H]⁺ | |
| | 7.15-7.07 (m, 1H), 5.49-5.33 (m, 2H), 4.34-4.09 (m, 2H), | | |
| | 2.33-2.19 (m, 1H), 2.07-1.85 (m, 5H), 1.71-1.61 (m, 1H), | | |
| | 1.53-1.39 (m, 1H) | | |
| 212 | Data provided above | | |
| 213 | (400 MHz, DMSO-d₆) δ 9.12 (s, 1H), 8.66 (s, 1H), 8.22 (s, | m/z 560.2 | >99% |
| | 1H), 7.76-7.67 (m, 3H), 7.57-7.43 (m, 3H), 7.08 (d, 1H), | [M + H]⁺ | |
| | 5.52-5.44 (m, 1H), 5.35 (d, 1H), 5.29 (d, 1H), 4.55-4.38 | | |
| | (m, 1H), 4.01-3.92 (m, 1H), 3.91-3.77 (m, 1H), 2.49-2.40 | | |
| | (m, 1H), 2.35-2.21 (m, 1H), 2.17-2.08 (m, 1H), 1.95-1.84 | | |
| | (m, 1H), 1.76-1.67 (m, 1H), 1.54-1.38 (m, 1H) | | |
| 214 | (400 MHz, DMSO-d₆) δ 8.99 (s, 1H), 8.52 (s, 1H), 8.21 (s, | m/z 560.1 | 90% |
| | 2H), 7.75-7.66 (m, 3H), 7.58-7.43 (m, 3H), 7.07 (d, 1H), | [M + H]⁺ | |
| | 5.47 (d, 1H), 5.34 (d, 1H), 5.27 (d, 1H), 4.44-4.34 (m, | | |
| | 1H), 4.01-3.86 (m, 2H), 2.46-2.37 (m, 1H), 2.23-2.08 (m, | | |
| | 2H), 1.95-1.85 (m, 1H), 1.74-1.63 (m, 1H), 1.53-1.37 (m, | | |
| | 1H) | | |
| 215 | (400 MHz, CDCl₃) δ 8.91 (s, 1H), 8.22 (s, 1H), 7.89-7.76 | m/z 560.5 | >99% |
| | (m, 2H), 7.60 (s, 1H), 7.52 (s, 1H), 7.39-7.31 (m, 2H), | [M + H]⁺ | |
| | 7.20-7.09 (m, 1H), 7.00-6.92 (m, 1H), 6.88-6.80 (m, 1H), | | |
| | 5.29-5.12 (m, 1H), 4.96-4.82 (m, 1H), 4.49-4.21 (m, 3H), | | |
| | 2.43-2.28 (m, 2H), 2.09-1.93 (m, 2H), 1.83-1.72 (m, 1H), | | |
| | 1.65-1.56 (m, 1H) | | |
| 216 | (400 MHz, DMSO-d₆) δ 10.73 (s, 1H), 9.99 (s, 1H), 9.08 | m/z 558.3 | >99%* |
| | (s, 1H), 8.46 (s, 1H), 8.36 (s, 1H), 7.87-7.74 (m, 2H), | [M + H]⁺ | |
| | 7.64-7.54 (m, 2H), 7.11-7.01 (m, 1H), 6.61-6.51 (m, 1H), | | |
| | 5.35-5.26 (m, 2H), 4.96-4.85 (m, 2H), 4.63-4.49 (m, 1H), | | |
| | 4.03-3.91 (m, 1H), 2.25-2.10 (m, 3H), 1.92-1.78 (m, 3H), | | |
| | 1.57-1.42 (m, 2H) | | |
| 217 | (400 MHz, CD₃OD) δ 8.63 (s, 1H), 8.57 (s, 1H), 8.16-8.03 | m/z 561.4 | >99% |
| | (m, 1H), 7.87-7.76 (m, 2H), 7.75-7.62 (m, 2H), 7.46-7.34 | [M + H]⁺ | |
| | (m, 3H), 7.09 (d, 1H), 4.45 (t, 1H), 4.15-4.04 (m, 1H), | | |
| | 3.92 (s, 3H), 3.87-3.74 (m, 1H), 2.78-2.60 (m, 1H), 2.27- | | |
| | 2.12 (m, 2H), 2.04-1.93 (m, 1H), 1.85-1.69 (m, 1H), 1.62- | | |
| | 1.48 (m, 1H) | | |
| 218 | (400 MHz, CD₃OD) δ 8.64 (s, 1H), 8.57 (s, 1H), 8.51 (d, | m/z 532.4 | >99% |
| | 1H), 8.04 (s, 2H), 7.81 (s, 1H), 7.74-7.63 (m, 2H), 7.58 | [M + H]⁺ | |
| | (dd, 1H), 7.45-7.34 (m, 2H), 4.61-4.46 (m, 1H), 4.17-4.08 | | |
| | (m, 1H), 3.93-3.79 (m, 1H), 2.74-2.60 (m, 1H), 2.29-2.21 | | |
| | (m, 1H), 2.11-1.95 (m, 2H), 1.95-1.77 (m, 1H), 1.65-1.48 | | |
| | (m, 1H) | | |
| 219 | (400 MHz, DMSO-d₆) δ 8.99-8.88 (m, 3H), 8.70 (s, 1H), | m/z 505.0 | >99% |
| | 8.60 (d, 1H), 8.25 (s, 1H), 8.07-8.02 (m, 1H), 7.76-7.72 | [M + H]⁺ | |
| | (m, 1H), 7.65 (d, 1H), 7.41 (t, 1H), 4.96 (d, 1H), 4.48-4.26 | | |
| | (m, 2H), 4.13-3.98 (m, 1H), 2.97-2.88 (m, 1H), 2.84-2.75 | | |
| | (m, 1H), 2.43-2.25 (m, 2H), 1.97-1.66 (m, 5H), 1.07-0.97 | | |
| | (m, 6H) | | |
| 220 | (400 MHz, DMSO-d₆) δ 14.25 (s, 1H), 9.04 (s, 1H), 8.66 | m/z 530.5 | >99% |
| | (t, 1H), 8.45 (s, 1H), 7.97 (d, 1H), 7.86-7.76 (m, 2H), | [M + H]⁺ | |
| | 7.76-7.67 (m, 2H), 7.59-7.43 (m, 3H), 4.49-4.34 (m, 1H), | | |
| | 4.26-4.11 (m, 1H), 3.86-3.76 (m, 1H), 3.16 (s, 3H), 2.80- | | |
| | 2.64 (m, 1H), 2.43-2.30 (m, 1H), 2.25-2.13 (m, 1H), 2.10- | | |
| | 1.95 (m, 2H), 1.91-1.80 (m, 1H) | | |
| 221 | (400 MHz, DMSO-d₆) δ 14.27 (s, 1H), 9.04 (s, 1H), 8.70- | m/z 530.5 | 93% |
| | 8.62 (m, 1H), 8.46 (s, 1H), 7.98 (d, 1H), 7.86-7.76 (m, | [M + H]⁺ | |
| | 2H), 7.76-7.67 (m, 2H), 7.58-7.41 (m, 3H), 4.50-4.35 (m, | | |
| | 1H), 4.26-4.11 (m, 1H), 3.88-3.74 (m, 1H), 3.17 (s, 3H), | | |
| | 2.80-2.64 (m, 1H), 2.45-2.29 (m, 1H), 2.27-2.13 (m, 1H), | | |
| | 2.10-1.93 (m, 2H), 1.95-1.83 (m, 1H) | | |
| 222 | (400 MHz, DMSO-d₆) δ 14.80-14.01 (m, 1H), 8.82-8.19 | m/z 534.2 | >99% |
| | (m, 4H), 8.14-7.93 (m, 2H), 7.85 (s, 1H), 7.76-7.69 (m, | [M + H]⁺ | |
| | 1H), 7.45-7.24 (m, 3H), 5.08-4.93 (m, 1H), 4.54-4.44 (m, | | |
| | 1H), 4.21-4.11 (m, 1H), 3.75-3.59 (m, 1H), 2.35-2.23 (m, | | |
| | 1H), 1.95-1.83 (m, 2H), 1.68-1.55 (m, 1H), 1.53-1.42 (m, | | |
| | 1H), 1.21-1.06 (m, 1H) | | |
| 223 | (400 MHz, CDCl₃) δ 8.51 (dd, 1H), 8.30 (s, 1H), 8.04- | m/z 572.0 | >99% |
| | 7.96 (m, 2H), 7.88-7.84 (m, 2H), 7.76 (s, 1H), 7.57-7.50 | [M + H]⁺ | |
| | (m, 1H), 7.43 (dd, 1H), 7.28 (s, 1H), 7.19-7.12 (m, 3H), | | |
| | 4.83-4.68 (m, 1H), 4.41-4.32 (m, 1H), 4.27 (s, 2H), 3.89- | | |
| | 3.78 (m, 3H), 3.21 (s, 3H), 2.55-2.22 (m, 5H), 1.65-1.57 | | |
| | (m, 2H) | | |
| 224 | (400 MHz, CDCl₃) δ 8.84 (d, 2H), 8.73 (s, 1H), 8.53-8.42 | m/z 583.2 | >99% |
| | (m, 2H), 7.98 (d, 1H), 7.90-7.85 (m, 2H), 7.56-7.49 (m, | [M + H]⁺ | |
| | 1H), 7.46-7.41 (m, 1H), 7.22-7.11 (m, 4H), 4.85-4.72 (m, | | |
| | 1H), 4.44-4.34 (m, 1H), 4.31-4.21 (m, 2H), 3.95-3.86 (m, | | |
| | 1H), 3.83 (t, 2H), 3.24 (s, 3H), 2.56-2.41 (m, 3H), 2.37- | | |
| | 2.26 (m, 1H), 1.69-1.63 (m, 2H) | | |
| 225 | (400 MHz, DMSO-d₆) δ 14.19 (s, 1H), 8.65-8.27 (m, 3H), | m/z 533.1 | >99% |
| | 7.97 (d, 1H), 7.81 (d, 1H), 7.73-7.63 (m, 2H), 7.51-7.38 | [M + H]⁺ | |
| | (m, 3H), 7.36-7.23 (m, 2H), 5.14 (d, 1H), 4.30-4.16 (m, | | |
| | 1H), 4.04-3.90 (m, 1H), 3.71-3.60 (m, 1H), 2.46-2.33 (m, | | |
| | 1H), 2.21-2.08 (m, 1H), 1.91-1.80 (m, 2H), 1.69-1.54 (m, | | |
| | 1H), 1.52-1.35 (m, 1H) | | |
| 226 | (400 MHz, DMSO-d₆) δ 14.14 (s, 1H), 9.04 (s, 1H), 8.73- | m/z 500.2 | >99%* |
| | 8.62 (m, 1H), 8.46 (s, 1H), 8.02-7.91 (m, 1H), 7.86-7.76 | [M + H]⁺ | |
| | (m, 2H), 7.74-7.65 (m, 2H), 7.58-7.41 (m, 3H), 4.26-4.11 | | |
| | (m, 1H), 4.01-3.88 (m, 1H), 2.70-2.57 (m, 1H), 2.38-2.14 | | |
| | (m, 1H), 2.09-1.79 (m, 4H), 1.71-1.56 (m, 1H), 1.55-1.37 | | |
| | (m, 1H) | | |
| 227 | (400 MHz, DMSO-d₆) δ 14.26 (s, 1H), 11.03 (s, 1H), 9.00 | m/z 532.0 | >99%* |
| | (d, 1H), 8.60 (d, 1H), 8.48-8.35 (m, 2H), 8.00 (s, 1H), 7.94 | [M + H]⁺ | |
| | (d, 1H), 7.81-7.69 (m, 2H), 7.51-7.37 (m, 1H), 6.88 (d, | | |
| | 1H), 6.82 (t, 1H), 4.20-4.06 (m, 1H), 3.98-3.84 (m, 1H), | | |
| | 2.69-2.56 (m, 1H), 2.30-2.17 (m, 1H), 2.15-2.00 (m, 1H), | | |
| | 1.98-1.79 (m, 3H), 1.71-1.57 (m, 1H), 1.48-1.31 (m, 1H) | | |
| 228 | (400 MHz, DMSO-d₆) δ 14.16 (s, 1H), 8.55 (s, 1H), 8.38 | m/z 559.3 | >99%* |
| | (s, 1H), 8.29-8.12 (m, 1H), 7.93 (d, 1H), 7.75 (d, 1H), 7.58 | [M + H]⁺ | |
| | (t, 1H), 7.45 (d, 1H), 7.38-7.27 (m, 4H), 7.14 (t, 1H), 4.79 | | |
| | (m , 1H), 4.20-4.01 (m, 3H), 3.90-3.71 (m, 1H), 3.67-3.57 | | |
| | (m, 2H), 2.41-2.08 (m, 3H), 2.00-1.65 (m, 3H), 1.52-1.31 | | |
| | (m, 2H) | | |
| 229 | (400 MHz, DMSO-d₆) δ 14.16 (s, 1H), 8.59 (s, 2H), 8.38 | m/z 575.2 | >99%* |
| | (s, 1H), 7.93 (d, 1H), 7.75 (d, 1H), 7.62-7.51 (m, 3H), 7.45 | [M + H]⁺ | |
| | (d, 1H), 7.37-7.27 (m, 2H), 7.14 (t, 1H), 4.92-4.64 (m, | | |
| | 1H), 4.22-4.02 (m, 3H), 3.88-3.72 (m, 1H), 3.69-3.58 (m, | | |
| | 2H), 2.41-2.10 (m, 3H), 1.98-1.68 (m, 3H), 1.52-1.29 (m, | | |
| | 2H) | | |
| 230 | Data provided above | | |
| 231 | Data provided above | | |
| 232 | Data provided above | | |
| 233 | (400 MHz, DMSO-d₆) δ 14.34 (s, 1H), 9.28-9.04 (m, 1H), | m/z 560.6 | N/A |
| | 8.74-8.62 (m, 1H), 8.43 (s, 1H), 8.41-8.29 (m, 1H), 7.94 | [M + H]⁺ | |
| | (d, 1H), 7.86-7.73 (m, 2H), 7.61-7.51 (m, 2H), 7.51-7.44 | | |
| | (m, 1H), 7.35-7.25 (m, 1H), 7.13 (t, 1H), 5.17-4.62 (m, | | |
| | 2H), 4.43-4.01 (m, 4H), 3.76-3.56 (m, 2H), 3.02-2.56 (m, | | |
| | 2H), 2.47-2.33 (m, 1H), 2.28-2.16 (m, 1H), 2.11-1.92 (m, | | |
| | 1H), 1.84-1.42 (m, 1H) | | |
| 234 | (400 MHz, CD₃OD) δ 8.99-8.92 (m, 2 H), 8.89 (s, 1 H), | m/z 584.3 | 99% |
| | 8.64-8.52 (m, 1 H), 8.50-8.43 (m, 1 H), 8.18-8.01 (m, 1 | [M + H]⁺ | |
| | H), 7.84-7.76 (m, 1 H), 7.68-7.59 (m, 2 H), 7.57-7.49 (m, | (Rt = 1.542 | |
| | 1H), 7.46-7.40 (m, 1 H), 7.35-7.28 (m, 1 H), 7.24-7.17 (m, | min) | |
| | 1 H), 4.62-4.41 (m, 1 H), 4.37-4.11 (m, 3 H), 3.85-3.74 | | |
| | (m, 2 H), 3.18-3.06 (m, 1 H), 3.04-2.73 (m, 2 H) , 2.71- | | |
| | 2.01 (m, 6 H) | | |
| 235 | (400 MHz, CD₃OD) δ 8.98-8.92 (m, 2 H), 8.89 (s, 1 H), | m/z 584.3 | 99% |
| | 8.64-8.52 (m, 1 H), 8.50-8.43 (m, 1 H), 8.10 (br s, 1 H), | [M + H]⁺ | |
| | 7.84-7.76 (m, 1 H), 7.68-7.59 (m, 2 H), 7.57-7.49 (m, 1H), | (Rt = 2.410 | |
| | 7.46-7.40 (m, 1 H), 7.36-7.28 (m, 1 H), 7.24-7.17 (m, 1 | min) | |
| | H), 4.60-4.44 (m, 1 H), 4.36-4.09 (m, 3 H), 3.84-3.74 (m, | | |
| | 2 H), 3.16-3.06 (m, 1 H), 3.04-2.73 (m, 2 H) , 2.68-2.01 | | |
| | (m, 6 H) | | |
| 236 | (400 MHz, CD₃OD) δ 8.95-8.94 (m, 2 H), 8.89 (s, 1 H), | m/z 568.3 | 99% |
| | 8.65 (br s, 1 H), 8.51-8.40 (m, 1 H), 7.92-7.76 (m, 2 H), | [M + H]⁺ | |
| | 7.69-7.61 (m, 1 H), 7.57-7.49 (m, 1 H), 7.47-7.42 (m, 1 | (Rt = 1.418 | |
| | H), 7.41-7.28 (m, 2 H), 7.25-7.17 (m, 1 H), 4.60-4.43 (m, | min) | |
| | 1 H), 4.38-4.12 (m, 3 H), 3.83-3.73 (m, 2 H), 3.21-3.08 | | |
| | (m, 1 H), 3.07-2.56 (m, 3 H), 2.55-2.34 (m, 4 H) , 2.13 (br | | |
| | s, 1 H) | | |
| 237 | (400 MHz, CD₃OD) δ 8.95-8.94 (m, 2 H), 8.90 (s, 1 H), | m/z 568.3 | 100% |
| | 8.65 (br s, 1 H), 8.52-8.41 (m, 1 H), 7.93-7.75 (m, 2 H), | [M + H]⁺ | |
| | 7.69-7.61 (m, 1 H), 7.59-7.49 (m, 1 H), 7.47-7.42 (m, 1 | (Rt = 0.958 | |
| | H), 7.41-7.28 (m, 2 H), 7.25-7.17 (m, 1 H), 4.60-4.44 (m, | min) | |
| | 1 H), 4.40-4.10 (m, 3 H), 3.79 (br s, 2 H), 3.15-3.08 (m, 1 | | |
| | H), 3.07-2.56 (m, 3 H), 2.55-2.34 (m, 4 H) , 2.13 (br s, 1 | | |
| | H) | | |
| 238 | (400 MHz, DMSO-d₆) δ 14.30 (s, 1H), 8.51 (s, 1H), 7.94- | m/z 577.2 | >99%* |
| | 7.86 (m, 1H), 7.78-7.72 (m, 1H), 7.70-7.61 (m, 3H), 7.52- | [M + H]⁺ | |
| | 7.38 (m, 5H), 4.53-4.36 (m, 2H), 4.30-4.02 (m, 2H), 2.42- | | |
| | 2.28 (m, 1H), 2.28-1.87 (m, 4H), 1.82-1.72 (m, 1H), 1.68- | | |
| | 1.38 (m, 2H) | | |
| 239 | (400 MHz, DMSO-d₆) δ 8.99 (d, 2H), 8.91 (s, 1H), 8.42 | m/z 529.5 | >99%* |
| | (dd, 1H), 8.15 (d, 1H), 7.99-7.93 (m, 1H), 7.90-7.84 (m, | [M + H]⁺ | |
| | 3H), 7.65-7.59 (m, 1H), 7.48 (t, 1H), 7.46-7.38 (m, 2H), | | |
| | 4.55-4.36 (m, 3H), 4.23-4.11 (m, 1H), 2.49-2.40 (m, 1H), | | |
| | 2.28-2.09 (m, 3H), 2.00-1.92 (m, 1H), 1.84-1.76 (m, 1H), | | |
| | 1.73-1.50 (m, 2H) | | |
| 240 | (400 MHz, DMSO-d₆) δ 14.41 (s, 1H), 8.47 (s, 1H), 8.24 | m/z 593.4 | >99% |
| | (s, 1H), 8.03-7.94 (m, 1H), 7.88-7.59 (m, 3H), 7.52-7.24 | [M + H]⁺ | |
| | (m, 5H), 5.22 (d, 1H), 4.55-4.24 (m, 3H), 4.00-3.84 (m, | | |
| | 2H), 2.68-2.54 (m, 1H), 2.20-2.06 (m, 1H), 2.04-1.86 (m, | | |
| | 2H), 1.79-1.67 (m, 1H), 1.67-1.51 (m, 1H) | | |
| 241 | (400 MHz, CDCl₃) δ 8.32 (s, 1H), 8.02-7.97 (m, 1H), 7.86 | m/z 583.0 | >99% |
| | (d, 1H), 7.77 (s, 1H), 7.58-7.51 (m, 1H), 7.51-7.45 (m, | [M + H]⁺ | |
| | 1H), 7.44-7.38 (m, 2H), 7.28 (s, 1H), 7.25-7.20 (m, 1H), | | |
| | 7.19-7.12 (m, 2H), 4.88-4.76 (m, 1H), 4.70-4.60 (m, 1H), | | |
| | 4.39 (s, 2H), 4.32-4.21 (m, 2H), 3.94-3.86 (m, 1H), 3.86- | | |
| | 3.75 (m, 2H), 3.22 (s, 3H), 2.61-2.51 (m, 1H), 2.49-2.40 | | |
| | (m, 1H), 2.39-2.19 (m, 3H), 1.90-1.75 (m, 2H) | | |
| 242 | Data provided above | | |
| 243 | (400 MHz, CD₃OD) δ 9.00-8.96 (m, 2 H), 8.89-8.85 (m, 1 | m/z 579.3 | 98% |
| | H), 8.50-8.41 (m, 1 H), 7.83-7.75 (m, 1 H), 7.67-7.55 (m, | [M + H]⁺ | |
| | 2 H), 7.54-7.49 (m, 1 H), 7.47-7.39 (m, 2 H), 7.37-7.27 | (Rt = 2.167 | |
| | (m, 2 H), 7.22-7.15 (m, 1 H), 4.65-4.55 (m, 3 H), 4.46- | min) | |
| | 4.24 (m, 2H), 4.21-4.06 (m, 1 H), 3.80-3.69 (m, 2 H), | | |
| | 3.52-3.33 (m, 1 H), 3.17-3.10 (m, 2 H), 2.99-2.70 (m, 2 | | |
| | H), 2.67-2.57 (m, 1 H), 2.57-2.25 (m, 3 H) | | |
| 244 | (400 MHz, CD₃OD) δ 8.99-8.97 (m, 2 H), 8.91-8.87 (m, 1 | m/z 579.3 | 96% |
| | H), 8.55-8.47 (m, 1 H), 7.90-7.80 (m, 1 H), 7.73-7.64 (m, | [M + H]⁺ | |
| | 1 H), 7.64-7.53 (m, 2 H), 7.51-7.42 (m, 2 H), 7.41-7.30 | (Rt = 1.939 | |
| | (m, 2 H), 7.26-7.17 (m, 1 H), 4.84-4.75 (m, 2 H), 4.69- | min) | |
| | 4.52 (m, 3 H), 4.47-4.32 (m, 1 H), 4.23-4.09 (m, 1 H), | | |
| | 4.06-3.92 (m, 1 H), 3.89-3.72 (m, 2 H), 3.69-3.52 (m, 2 | | |
| | H), 3.48-3.32 (m, 1 H), 3.19-2.89 (m, 4 H) | | |

*(%ee is based on enantiopurity of chiral intermediate)

### BCAT2 In Vitro BCAT2 Cell Inhibition Using Engineered A549 Cells

Endogenous Methylmalonyl-CoA Mutase (MUT) and Branched Chain Aminotransferase 1 (BCAT1) in A549 cells was knocked out by CRISPR/CAS9 using the targeting system from TransOMIC Technologies. The following guide sequence was cloned into pCLIP-ALL-EFS-Blast vector to target human MUT: ATCATGTAAGAACCTCCCCA (SEQ ID NO: 35). The following guide sequence was cloned into pCLIP-ALL-EFS-Puro vector to target human BCAT1: TACTGGATAAATGAAGATGG (SEQ ID NO: 36). The resulting plasmids were co-transfected into 293T cells with VSV-G, P-PEX, and Fugene-6. Lentivirus was harvested and A549 cells were first transduced with lentivirus targeting MUT and polybrene. After genetic selection with blasticidin clonal selection was performed and validated via western blot analysis to confirm MUT was no longer expressed. These MUT KO cells were then transduced with lentivirus targeting BCAT1 and polybrene. After selection with puromycin clonal selection was performed and clones were validated via western blot analysis to confirm BCAT1 was no longer expressed in these cells. The result was an engineered A549 cell line that expressed endogenous BCAT2.

For KMV cell assays, on day 1, 25,000 cells/100 μL EMEM (10% FBS) were seeded in each well of a 96-well plate. On day 2, medium was replaced with 50 μL fresh EMEM (10% dialyzed FBS) containing 2× the assay concentration of experimental compound. After 15 minutes, 50 μL of 800 uM ¹⁵N,¹³C₆-Isoleucine in EMEM (10% dialyzed FBS) was added to each well and incubated for 24 hours. On day 3, 50 μL of media from each well was collected and transferred to another 96-well plate for ¹³C₆-3-Methyl-2-Oxopentanoic Acid (KMV) analysis via LC-MS/MS. In the remaining assay wells 50 μL of Promega Glo CTG reagent was added and luminescence was measured as a cell viability readout.

For LC-MS/MS analysis 5 μL of cell assay media from each well was transferred to a 96-well plate and 45 μL of ACN:0.1% NaOH (50:50, v/v)⁺0.20 nM d8-KMV was added and mixed on a shaker. 15 μL was transferred to a new 96-well plate and 15 μL of 10 mg/mL PFBHA solution was added and mixed on the shaker. The reaction was placed on ice for 30 minutes and quenched with 10 μL of ACN: Acetone (50:50, v/v) on a shaker. The resulting mixture was diluted with 100 μL of ACN:0.1% NaOH (50:50, v/v) on a shaker. 10 μL of sample were injected on the Shimadzu 20AD LC-MS/MS to quantify ¹³C₆-KMV.

Specific compounds disclosed herein were tested in the foregoing assay and they were determined to inhibit BCAT2 in the cell with an IC₅₀ according to the following scores: (A) less than or equal to 0.100 μM, (B) greater than 0.100 and less than or equal to 0.500 μM, (C) greater than 0.500 and less than or equal to 1.000 PM, (d) greater than 1.000 μM, (E) no fit, and (NT) not tested, as shown in Table 6 below. Where a compound was tested multiple times, the average value of the tests is reported.

### BCAT2 In Vitro Enzyme Inhibition Assay

### Procedure:

2×Buffer: 200 mM Tris pH 8.0, 300 mM NaCl, 0.1% BSA
1.67×hBCAT2 Enzyme Mix: 0.33 μg/mL hBCAT2, 1× Buffer
2.5×Substrate Mix: 460 M L-leucine, 420 M α-KetoGlutarate, 10 U/mL glutamate dehydrogenase, 10 mM NAD, 30 g/mL diaphorase, 20 m Resazurin

BCAT2 enzyme stock solution was diluted to prepare a 1.67×hBCAT2 Enzyme Mix. 1 μL of test compound was first added to the reaction wells followed by 30 μL of 1.67×hBCAT2 Enzyme Mix and incubated at room temperature (25° C.) for one hour. Meanwhile the Substrate Mix was prepared. The reaction was initiated by the addition of 20 μL of the 2.5×Substrate Mix, bringing the final reaction mix of all components to 1×. Reactions were quenched after one hour by the addition of 25 μL of 6% SDS and reaction progress was measured via fluorescence read (Ex 540/Em 590).

### Test Compound Preparation:

Test compounds were prepared at 50×final concentration in DMSO, 1 to 3 dilutions were made for 10 points. The compounds were added to the assay as a 1 to 50 dilution (1 μL in 50 μL) to yield a top assay final concentration of 100 μM, decreasing 3-fold for 10 points. Each compound dilution series test also included an assay max (1 μL DMSO Added in lieu of compound, Full Reaction Components Included) and Min (1 μL DMSO Added in lieu of compound, 30 μl of 1.67×hBCAT2 Enzyme Mix replated by 30 μL of 1×Buffer). All tests were performed in technical duplicate.

Specific compounds disclosed herein were tested in the foregoing assay and they were determined to inhibit the BCAT2 enzyme with an IC₅₀ according to the following scores: (A) less than or equal to 0.100 μM, (B) greater than 0.100 and less than or equal to 0.500 μM, (C) greater than 0.500 and less than or equal to 1.000 μM, (d) greater than 1.000 μM, (E) no fit, and (NT) not tested, as shown in Table 6 below. Where a compound was tested multiple times, the average value of the tests is reported.

TABLE 6

| | BCAT2 Cell | BCAT2 Enzyme |
|---|---|---|
| Ex. No. | Inhibition | Inhibition |
| 1 | A | A |
| 2 | D | A |
| 3 | B | A |
| 4 | C | A |
| 5 | A | A |
| 6 | A | A |
| 7 | A | A |
| 8 | NT | A |
| 9 | NT | A |
| 10 | C | A |
| 11 | C | A |
| 12 | E | D |
| 13 | A | A |
| 14 | A | A |
| 15 | A | A |
| 16 | B | A |
| 17 | E | A |
| 18 | A | A |
| 19 | B | A |
| 20 | E | D |
| 21 | D | B |
| 22 | A | A |
| 23 | A | A |
| 24 | D | C |
| 25 | B | A |
| 26 | B | A |
| 27 | A | A |
| 28 | E | B |
| 29 | A | A |
| 30 | A | A |
| 31 | A | A |
| 32 | D | B |
| 33 | C | A |
| 34 | A | A |
| 35 | C | B |
| 36 | A | A |
| 37 | C | A |
| 38 | E | D |
| 39 | A | A |
| 40 | B | A |
| 41 | NT | B |
| 42 | NT | A |
| 43 | NT | C |
| 44 | NT | D |
| 45 | A | A |
| 46 | E | A |
| 47 | NT | A |
| 48 | NT | A |
| 49 | B | A |
| 50 | NT | A |
| 51 | NT | A |
| 52 | NT | A |
| 53 | A | A |
| 54 | A | A |
| 55 | D | A |
| 56 | A | A |
| 57 | C | A |
| 58 | A | A |
| 59 | A | A |
| 60 | NT | A |
| 61 | NT | A |
| 62 | NT | A |
| 63 | NT | A |
| 64 | NT | A |
| 65 | NT | A |
| 66 | NT | A |
| 67 | NT | A |
| 68 | A | A |
| 69 | A | A |
| 70 | A | A |
| 71 | A | A |
| 72 | A | A |
| 73 | A | A |
| 74 | B | A |
| 75 | NT | A |
| 76 | A | A |
| 77 | B | A |
| 78 | B | A |
| 79 | NT | A |
| 80 | B | A |
| 81 | B | A |
| 82 | A | A |
| 83 | A | A |
| 84 | A | A |
| 85 | B | A |
| 86 | B | A |
| 87 | NT | D |
| 88 | B | A |
| 89 | A | A |
| 90 | B | A |
| 91 | NT | D |
| 92 | NT | D |
| 93 | B | A |
| 94 | NT | D |
| 95 | E | D |
| 96 | E | D |
| 97 | NT | A |
| 98 | A | A |
| 99 | NT | A |
| 100 | A | A |
| 101 | NT | A |
| 102 | A | A |
| 103 | NT | A |
| 104 | D | C |
| 105 | A | A |
| 106 | A | A |
| 107 | E | D |
| 108 | E | A |
| 109 | NT | C |
| 110 | NT | A |
| 111 | NT | A |
| 112 | NT | D |
| 113 | A | A |
| 114 | A | A |
| 115 | E | B |
| 116 | A | A |
| 117 | C | A |
| 118 | A | A |
| 119 | A | A |
| 120 | A | A |
| 121 | NT | B |
| 122 | A | A |
| 123 | E | A |
| 124 | E | A |
| 125 | E | A |
| 126 | NT | A |
| 127 | NT | A |
| 128 | B | A |
| 129 | D | B |
| 130 | A | A |
| 131 | E | D |
| 132 | D | A |
| 133 | D | B |
| 134 | B | A |
| 135 | E | C |
| 136 | B | A |
| 137 | D | B |
| 138 | E | D |
| 139 | A | A |
| 140 | NT | C |
| 141 | A | A |
| 142 | NT | B |
| 143 | A | A |
| 144 | NT | B |
| 145 | B | A |
| 146 | NT | B |
| 147 | NT | B |
| 148 | NT | A |
| 149 | NT | C |
| 150 | B | A |
| 151 | NT | B |
| 152 | NT | B |
| 153 | B | A |
| 154 | NT | D |
| 155 | E | A |
| 156 | E | D |
| 157 | E | A |
| 158 | E | A |
| 159 | NT | D |
| 160 | E | D |
| 161 | E | A |
| 162 | E | C |
| 163 | E | A |
| 164 | E | A |
| 165 | E | A |
| 166 | E | A |
| 167 | A | A |
| 168 | A | A |
| 169 | A | A |
| 170 | A | A |
| 171 | A | A |
| 172 | A | A |
| 173 | A | A |
| 174 | A | A |
| 175 | A | A |
| 176 | B | A |
| 177 | B | A |
| 178 | A | A |
| 179 | A | A |
| 180 | A | A |
| 181 | A | A |
| 182 | A | A |
| 183 | A | A |
| 184 | A | A |
| 185 | A | A |
| 186 | A | A |
| 187 | B | A |
| 188 | B | A |
| 189 | A | A |
| 190 | A | NT |
| 191 | NT | NT |
| 192 | B | A |
| 193 | NT | A |
| 194 | A | A |
| 195 | A | A |
| 196 | E | A |
| 197 | E | A |
| 198 | E | B |
| 199 | E | A |
| 200 | A | A |
| 201 | A | A |
| 202 | A | A |
| 203 | NT | A |
| 204 | A | A |
| 205 | A | A |
| 206 | A | A |
| 207 | NT | A |
| 208 | A | A |
| 209 | A | A |
| 210 | NT | A |
| 211 | A | A |
| 212 | A | A |
| 213 | A | A |
| 214 | A | A |
| 215 | A | A |
| 216 | NT | D |
| 217 | D | A |
| 218 | B | A |
| 219 | B | A |
| 220 | NT | D |
| 221 | D | A |
| 222 | NT | B |
| 223 | E | B |
| 224 | E | A |
| 225 | E | A |
| 226 | B | A |
| 227 | B | A |
| 228 | NT | A |
| 229 | NT | A |
| 230 | A | A |
| 231 | E | A |
| 232 | E | B |
| 233 | NT | NT |
| 234 | E | A |
| 235 | E | D |
| 236 | NT | B |
| 237 | NT | D |
| 238 | A | A |
| 239 | A | A |
| 240 | B | A |
| 241 | B | A |
| 242 | NT | A |
| 243 | B | A |
| 244 | NT | C |

### Example 245. Preparation of BCAT2-KO Mice

Transcription of gRN: For in vitro transcription of sgRNA targeting exon 3 of the mouse BCAT2 gene, a template was amplified by PCR from a plasmid containing intact sgRNA structure, using a T7 promoter tagged forward primer (T7-sgRNA_F) and a reverse primer (sgRNA_R):

T7-sgRNA_F:
(SEQ ID NO: 12)
ttaatacgactcactataggNNNNNNNNNNNNNNNNNNNN
gttttagagctagaaatag
sgRNA_R:
(SEQ ID NO: 7)
AAAAGCACCGACTCGGTGCCAC

where NNNNNNNNNNNNNNNNNNNN denotes the sgRNA targeting sequences. Two sgRNA sequences were used in the construction of the BCAT2 KO:

Bcat2-sgRNA#1:
(SEQ ID NO: 8)
GTGGAGTGGAATAACAAGGC
Bcat2-sgRNA#2:
(SEQ ID NO: 9)
AGGCTGGATGGGGCCCACCA

After purification of the DNA templates, in vitro transcription was performed using the MEGAshortscript T7 kit and MEGAclear kit according to the manufacturer's protocol (Ambion). Cas9 mRNA was in vitro transcribed from pUC-cas9 vector using mMESSAGE mMACHINE T7 kit (Ambion).

Pronuclear injection: 10 to 15 C57BL/6 females (4-week-old) were injected with 5-10 IU of pregnant mare's serum gonadotrophin (PMSG) on day 1 to induce superovulation. 48 hours later, the mice were injected with 5-10 IU human chorionic gonadotrophin (HCG) and then housed with a C57BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C. in a 5% CO₂ incubator. Cas9 mRNA and sgRNAs (all 10 ng/μl) were injected into the pronucleus of the zygotes. Injected zygotes were then implanted into the oviduct of pseudo-pregnant foster mothers at 0.5 days post coitum (dpc).

Genotyping: Genomic DNAs were extracted from tails and toes of the 7-day-old pups and used in PCR amplification of the sequence around the targeting site with the following two primers:

Bcat2-GT-F2:
(SEQ ID NO: 10)
GGGTCTAAGGGAAAAGCCCG
Bcat2-GT-R2:
(SEQ ID NO: 11)
TATCGGTGTAAGATGCCCCC

The resultant PCR products were TA cloned and sequenced to identify F₀ mice, which were back crossed with wildtype C57BL/6 mice to identify F₁ mice with a germline transmitted mutation.

The INDEL generated had an adenine (A) deletion at position 245 (exon 3) of SEQ ID NO: 1, resulting in a stop codon at positions 344-346 (exon 4) of SEQ ID NO: 1. Exon 3 of the BCAT2 gene is positions 127-330 of SEQ ID NO: 1. Exon 4 of the BCAT2 gene is positions 331-441 of SEQ ID NO: 1.

### Western Blot Analysis of BCAT2 Expression in Muscle

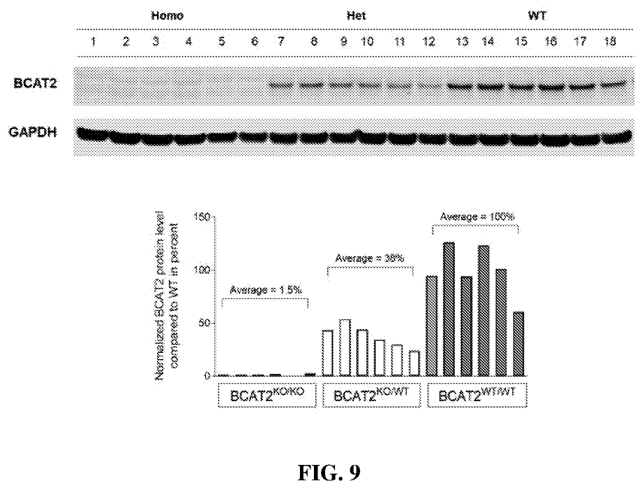

Hindleg muscles were collected from mice aged from 8 to 10 weeks old. Muscle lysates were prepared and BCAT2 expression measured via Western Blotting using a BCAT2 antibody (Abcam cat. No. ab197917; MW 44 kDa) at a dilution of 1:1000 and a GAPDH antibody (Cell Signaling Technology cat. No. 97166; MW 37 kDa) at a dilution of 1:3000. For quantification, a ratio of the band intensity of BCAT2 over GAPDH was first obtained for each sample. Then, an average of this BCAT2/GAPDH ratio in the WT group was calculated (averaged baseline value in WT) and used as the baseline for normalization. To calculate normalized percent change compared to WT, the BCAT2/GAPDH ratio of each sample was divided by the averaged baseline value in WT, followed by multiplying by 100 to obtain a percentage. As shown in FIG. 9, mice homozygous for the BCAT2 knockout had 1.5% normalized BCAT2 protein band intensity compared to WT, whereas BCAT2 protein band intensity in heterozygous mice was 38% of that in the wildtype.

### Example 246: Preparation of Mice with a Knockout in the Gene Encoding MMA-CoA Mutase (Mut-KO)

Transcription of gRNAs: For in vitro transcription of sgRNA targeting exon 1, a template was amplified by PCR from a plasmid containing intact sgRNA structure, using a T7 promoter tagged forward primer (T7-sqRNA_F) and a reverse primer (sgRNA_R):

T7-sgRNA_F:
(SEQ ID NO: 12)
ttaatacgactcactataggNNNNNNNNNNNNNNNNNNNN
gttttagagctagaaatag
sgRNA_R:
(SEQ ID NO: 13)
AAAAGCACCGACTCGGTGCCAC,

where NNNNNNNNNNNNNNNNNNNN denotes the sgRNA targeting sequences. One sgRNA sequence was used in the construction of MUT KO:

Mut-sgRNA#1:
(SEQ ID NO: 14)
GTGTGCCATATAAGGTCCTC

After purification of the DNA templates, in vitro transcription was performed using the MEGAshortscript T7 kit and MEGAclear kit according to the manufacturer's protocol (Ambion). BE4-Gam mRNA was in vitro transcribed from pUC-BE4-Gam vector using mMESSAGE mMACHINE T7 kit (Ambion).

Pronuclear injection: 10 to 15 C57BL/6 females (4-week-old) were injected with 5-10 IU of pregnant mare's serum gonadotrophin (PMSG) on day 1 to induce superovulation. 48 hours later, the mice were injected with 5-10 IU human chorionic gonadotrophin (HCG) and then housed with a C57BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C. in a 5% CO₂ incubator. BE4-Gam mRNA and sgRNA (all 10 ng/μl) were injected into the pronucleus of the zygotes. Injected zygotes were implanted into the oviduct of pseudo-pregnant foster mothers at 0.5 days post coitum (dpc).

Genotyping: Genomic DNAs were extracted from tails and toes of the 7-day-old pups and used in PCR amplification of the sequence around the targeting site with the following two primers:

MUT-KO-GT-F2:
(SEQ ID NO: 15)
AATGGGTTTCAGGGGGCTAAC
MUT-KO-GT-R2:
(SEQ ID NO: 16)
GGCATGATTTAAGCTCTCCTCC

The resultant PCR products were TA cloned and sequenced to identify F0 mice, which were back crossed with wildtype C57BL/6 mice to identify F1 mice with germline transmitted mutation.

The INDEL generated had a single G to A nucleotide change at position 311 of SEQ ID NO: 3, mutating the tryptophan (TGG) at position 59 of SEQ ID NO: 4 to a stop codon (TAG).

### Example 247: Preparation of Mice Comprising a Gene Encoding M698K MMA-CoA Mutase (M698K Mut)

Transcription of gRNAs: For in vitro transcription of an sgRNA targeting exon 11, a template was amplified by PCR from a plasmid containing intact sgRNA structure, using a T7 promoter tagged forward primer (T7-sqRNA_F) and a reverse primer (sgRNA_R): T7-sgRNA_F: ttaatacgactcactataggNNNNNNNNNNNNNNNNNNNNgttttagagctagaaatag (SEQ ID NO: 17)

sgRNA_R:
(SEQ ID NO: 18)
AAAAGCACCGACTCGGTGCCAC

where NNNNNNNNNNNNNNNNNNNN denotes the sgRNA targeting sequences. Two sgRNA sequences were used in the construction of MUT-M698K:

sgRNA#1:
(SEQ ID NO: 19)
CCAGATATCCTTGTCATGTG
sgRNA#2:
(SEQ ID NO: 20)
GATATCCTTGTCATGTGTGG

After purification of the DNA templates, in vitro transcription was performed using the MEGAshortscript T7 kit and MEGAclear kit according to the manufacturer's protocol (Ambion). Cas9 mRNA was in vitro transcribed from pUC-cas9 vector using mMESSAGE mMACHINE T7 kit (Ambion). A single stranded donor DNA was chemically synthesized with the following sequence:

(SEQ ID NO: 21)
5′cgtgtgtgagtatgtacgtgcgtgtacatgtatgcatgtgcaggagag
tacagagaaggaaaaatacctgtggtggaatcacgcccccGcacTtgaca
aggatatctggccgccccagggcggtgag3′

Pronuclear injection: 10 to 15 C57BL/6 females (4-week-old) were injected with 5-10 IU of pregnant mare's serum gonadotrophin (PMSG) on day 1 to induce superovulation. 48 hours later, the mice were injected with 5-10 IU human chorionic gonadotrophin (HCG) and then housed with a C57BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C. in a 5% CO₂ incubator. Cas9mRNA, sgRNA, and ssDNA (all 10 ng/μl) were injected into the pronucleus of the zygotes. Injected zygotes were then implanted into the oviduct of pseudo-pregnant foster mothers at 0.5 days post coitum (dpc).

Genotyping: Genomic DNAs were extracted from tails and toes of the 7-day-old pups and used in PCR amplification of the sequence around the targeting site with the following two primers:

M698K-GT-F1:
(SEQ ID NO: 22)
AAGGTCGCAGACCTCGTCTT
M698K-GT-R1:
(SEQ ID NO: 23)
GATTCCCAAGGACCGCACTC

The resultant PCR products were TA cloned and sequenced to identify F0 mice, which were back crossed with wildtype C57BL/6 mice to identify F1 mice with a germline transmitted mutation. The M698K mutation results from changing the thymine (T) at position 2228 of SEQ ID NO: 3 (methionine codon ATG) to adenine (A), resulting in a change from methionine to lysine (codon AAG) in the corresponding protein.

MUT^{M698K/KO} mice comprising a mutation that results in the M698K substitution at one locus of the MMA-CoA mutase gene, and a knockout of the MMA-CoA mutase gene at a second locus of the MMA-CoA mutase gene were prepared by crossing the MUT^{KO/KO} mice described in Example 2 with the MUT^{M698k/M698K} mice described above. MUT^{M698K/KO}BCAT2^{KO/KO} mice were prepared by crossing the MUT^{M698K/KO} mice described above with the BCAT2^{KO/KO} mice described in Example 245.

### Example 248: Preparation of Mice with a Knockout in the Gene Encoding Propionyl-CoA Carboxylase α Subunit (PCCA-KO)

Transcription of sgRNA: For in vitro transcription of an sgRNA targeting exon 5 of the propionyl-CoA carboxylase (PCCA) gene, a template was amplified by PCR from a plasmid containing intact sgRNA structure, using a T7 promoter tagged forward primer (T7-sqRNA_F) and a reverse primer (sgRNA_R):

T7-sgRNA_F:
(SEQ ID NO: 24)
ttaatacgactcactataggNNNNNNNNNNNNNNNNNNNNgttttagagc
tagaaatag
sgRNA_R:
(SEQ ID NO: 25)
AAAAGCACCGACTCGGTGCCAC

where NNNNNNNNNNNNNNNNNNNN denotes the sgRNA targeting sequences. One sgRNA sequence was used in the construction of PCCA-KO:

sgRNA#1:
(SEQ ID NO: 26)
TTGAGGTAGCTTTTACTGGT

After purification of the DNA templates, in vitro transcription was performed using the MEGAshortscript T7 kit and MEGAclear kit according to the manufacturer's protocol (Ambion). Cas9 mRNA was in vitro transcribed from pUC-cas9 vector using mMESSAGE mMACHINE T7 kit (Ambion).

Pronuclear injection: 10 to 15 C57BL/6J females (4-weeks-old) were injected with 5-10 IU of pregnant mare's serum gonadotrophin (PMSG) on day 1 to induce superovulation. 48 hours later, the mice were injected with 5-10 IU human chorionic gonadotrophin (HCG) and then housed with a C57BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C. in a 5% CO₂ incubator. Cas9mRNA and sgRNA (all 10 ng/μl) were injected into the pronucleus of the zygotes. Injected zygotes were then implanted into the oviduct of pseudo-pregnant foster mothers at 0.5 days post coitum (dpc).

Genotyping: Genomic DNAs were extracted from tails and toes of the 7-day-old pups and used in PCR amplification of the sequence around the targeting site with the following two primers:

PCCA KO-GT-F1:
(SEQ ID NO: 27)
GGGCCAGTCTGGTTCTATGT
PCCA KO-GT-R1:
(SEQ ID NO: 28)
TCGATACCATCGACCCCAAA

The resultant PCR products were TA cloned and sequenced to identify F3 mice, which were the result of heterozygous F2 crossing. Prior to that, F1 mice were backcrossed to C57BL/6J to identify F2 mice with germline transmitted mutation.

### Example 249: Preparation of Mice Comprising a Gene Encoding A134T Propionyl-CoA Carboxylase (PCCA-A134T)

Transcription of gRNA: For in vitro transcription of sgRNA targeting exon 5 of the propionyl-CoA carboxylase (PCCA) gene, a template was amplified by PCR from a plasmid containing intact sgRNA structure, using a T7 promoter tagged forward primer (T7-sqRNA_F) and a reverse primer (sgRNA_R):

T7-sgRNA_F:
(SEQ ID NO: 29)
ttaatacgactcactataggNNNNNNNNNNNNNNNNNNNNgttttagagc
tagaaatag
sgRNA_R:
(SEQ ID NO: 30)
AAAAGCACCGACTCGGTGCCAC

where NNNNNNNNNNNNNNNNNNNN denotes the sgRNA targeting sequences. One sgRNA sequence was used in the construction of PCCA-A134T:

sgRNA#1:
(SEQ ID NO: 31)
TTCGCTCAGACTCACAGCTT

After purification of the DNA templates, in vitro transcription was performed using the MEGAshortscript T7 kit and MEGAclear kit according to the manufacturer's protocol (Ambion). Cas9 mRNA was in vitro transcribed from pUC-cas9 vector using mMESSAGE mMACHINE T7 kit (Ambion).
A single stranded donor DNA was chemically synthesized with the following sequence:

(SEQ ID NO: 32)
5′gtctgtgttggcccagctcccaccagtaaaagctacctcaacatggat
gccatcatggaagccattaagaaaaccagggcccaaActgtgagtctgag
cgaacctatctactgcagctgtt3′

The single stranded donor DNA changes the guanine (G) at position 439 of SEQ ID NO: 5 to an adenine (A), resulting in the A134T substitution relative to SEQ ID NO: 6. The G439A substitution is shown capitalized and underlined in the sequence above.

Pronuclear injection: 10 to 15 C57BL/6J females (4-week-old) were injected with 5-10 IU of pregnant mare's serum gonadotrophin (PMSG) on day 1 to induce superovulation. 48 hours later, the mice were injected with 5-10 IU human chorionic gonadotrophin (HCG) and then housed with C57BL/6 male overnight. In the morning of day 4, female mice with copulation plugs were collected for zygote preparation. Zygote-cumulus complexes from the oviduct were collected from euthanized mice and suspended in hyaluronidase solution for several minutes until the cumulus cells fell off, which were then washed several times in M2 medium. The embryos were then placed in equilibrated M16 medium (medium covered with mineral oil) at 37° C. in a 5% CO₂ incubator. Cas9mRNA, sgRNA, and ssDNA (all 10 ng/μl) were injected into the pronucleus of the zygotes. Injected zygotes were then implanted into the oviduct of pseudo-pregnant foster mothers at 0.5 days post coitum (dpc).

Genotyping: Genomic DNAs were extracted from tails and toes of the 7-day-old pups and used in PCR amplification of the sequence around the targeting site with the following two primers:

A134T-GT-F1:
(SEQ ID NO: 33)
GGGCCAGTCTGGTTCTATGT
A134T-GT-R1:
(SEQ ID NO: 34)
TCGATACCATCGACCCCAAA

Changing the guanine (G) at position 439 of SEQ ID NO: 5 to an adenine (A) results in the A134T substitution relative to SEQ ID NO: 6.
The resultant PCR products were TA cloned and sequenced to identify F3 mice, which were the result of heterozygous F2 crossing. Prior to that F1 mice were backcrossed to C57BL/6J to identify F2 mice with a germline transmitted mutation.

PCCA^{A134T/KO} mice were prepared by crossing the PCCA^{KO/KO} mice described in Example 4 with the PCCA^{A134T/A134T} mice described above.

### Example 250. Analysis of Plasma Propionyl-Carnitine in the PCCA A134T/A134T Mouse Model

### Methods

20 μL of whole blood was collected from WT and PCCA A134T/A134T mice at 8 am on Day 1 and 12 am and 8 am on Day 2. Whole blood was centrifuged for 5 min. at 2000 g to obtain 10 μL of plasma. Analyte Propionyl-1-carnitine (PPC) and internal standard Propionyl-1-carnitine-(N-methyl-d3) were quantified using an AB Sciex Triple 6500+LC-MS/MS with an Acquity UPLC HSS T3 (1.8 m, 2.1×50 mm) column. Mobile phase A and B contained 0.1% Formic Acid in water and acetonitrile, respectively. The flow rate was held at 0.5 ml/min mobile phase A until 1.2 min where it was switched to 95% mobile phase B and held until 1.31 minutes, after which it returned to 100% mobile phase A until 1.5 minutes. The column temperature was maintained at 45° C. The sample injection volume was 1 μL. The MRM based mass spectrometric detection of PPC was achieved with turbo spray iondrive in a positive mode. The compound parameters for PPC and PPC—(N-methyl-d3) are shown in the Table 7 below.

TABLE 7
Acquisition parameters and retention
times of PPC and PPC-(N-methyl-d3)

| | Q1 | Q3 | Retention |
|---|---|---|---|
| Analytes | (m/z) | (m/z) | time (min) |
| PPC | 218 | 85.1 | 0.78 |
| PPC-(N-methyl-d3) | 221.1 | 85.1 | 0.78 |

Eight-point standard curve samples (1-3000 ng/mL) and QCs samples of PPC prepared in the surrogate matrix (0.05% BSA in PBS), and ENQC (C₅₇ mouse blank plasma) were used during the PPC analysis. The test plasma and control samples were prepared using the protein precipitation method. Briefly, the samples were diluted 15× with 20 ng/ml internal standard in methanol. After vortex mixing these samples were then centrifuged at 3220 g at 4° C. for 15 min. Sixty microliter of the supernatant was then transferred to 96 well plate, centrifuged for 5 min at 3220 g at 4° C., and directly injected for LC-MS/MS analysis. The standard curve had a coefficient of determination (R²) value>0.98 in a linear regression. The QC samples had precision and accuracy within ±20% of theoretical values. The peak area ratios of the analyte relative to the IS were used for PPC quantitation.

### Results

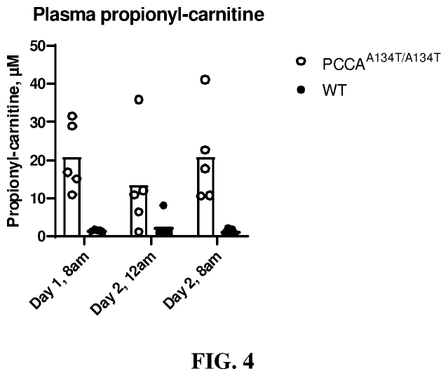

Plasma Propionyl-Carnitine (PPC) levels were elevated in PCCA^{134T/A134T} mice compared to WT mice when sampled over two days (FIG. 4). For example, homozygous PCCA^{A134T/A134T} mice had 10-30 μM plasma PPC at 8 am on day 1 compared to only 2 μM in WT mice. This trend was consistent across the Day 2 12 am and 8 am timepoints.

### Example 251. MMA Levels in Mut^{M698K/KO}, Mut^{M698K/M698K}, and Mut^{M698K/WT} Mice

### Methods

Plasma and urine MMA levels were measured in Mut^{M698}K mutant lines. Mice aging from 7.6 to 8.3 were selected for this experiment. Five mice were used in each group. Next day at 7 am, blood was drawn via the saphenous vein and immediately placed on ice. Blood was then processed into plasma via centrifugation. Plasma samples were snap-frozen in liquid nitrogen and stored at −80° C. until further analysis. Urine samples were also collected at the same timepoint from the collection chambers of the metabolic cages.

The plasma and urine samples collected from Mut^{M698K/M698K} and Mut^{M698K/KO} mouse models were quantified for the methylmalonic acid (MMA) levels using an LC-MS/MS method. A Waters Acquity UPLC coupled with an API 4000 triple quadrupole mass spectrometer was used for the quantitative analysis of MMA. The chromatographic separation of MMA was achieved on a Waters Acquity BEH amide (1.7 μm, 2.1×100 mm) column using a gradient method. The mobile phase consisted of a 95:5 mixture of 10 mM ammonium acetate buffer in water and acetonitrile, pH 10 (solvent A), and 95:5 mixture of 10 mM ammonium acetate buffer in acetonitrile and water, pH 10 (solvent B), and the flow rate was held at 0.6 ml/min. The gradient was started with pumps A and B supplying 88 and 12% until 0.3 minutes and then the composition of solvent A in the mobile phase was linearly decreased to 75% reaching 3.7 minutes and maintained at 75% until 4.0 minutes. The composition of solvent A in the mobile phase was then increased to 88% by 4.01 min and maintained until 4.5 minutes. The weak needle wash comprised of 10% acetonitrile in water. The strong needle wash included an equal volume mixture of acetonitrile, methanol, isopropyl alcohol, and water. The column oven and autosampler temperatures were maintained at 40 and 10° C., respectively. The sample injection volume was 4 μL. The MRM based mass spectrometric detection of MMA was achieved with electrospray ionization in a negative mode. The ¹³C₄ methylmalonic acid (¹³C₄-MMA) was used as an internal standard. The compound parameters (ion transitions, declustering potential, and collision energy) for MMA and ¹³C₄-MMA are shown in Table 8 below. General adjustment of the instrument was as follows: Turbo spray temperature was set to 550° C., ionization voltage was −4500 V, and nitrogen gas adjustment was set to 55 units for gas 1 and 2. The curtain gas was set to 20 and the CAD gas was set to 10 units and the entrance potential was −10 V.

TABLE 8
Acquisition parameters and retention
times of MMA and ¹³C₄ MMA

| | | | | | Retention |
|---|---|---|---|---|---|
| | Q1 | Q3 | Declustering | Collision | time |
| Analytes | (m/z) | (m/z) | potential (V) | energy (V) | (min) |
| MMA | 117 | 73.2 | −35 | −13 | 2.47 |
| ¹³C₄-MMA | 121 | 75.9 | −35 | −14 | 2.47 |

Eight-point standard curve samples (50-30000 ng/mL) and QCs samples of MMA prepared in the surrogate matrix (50% methanol in water), and ENQC (C57 mouse blank plasma or urine) were used during the MMA analysis. The test plasma and urine samples were prepared using the protein precipitation method. Briefly, the test samples were initially diluted using a surrogate matrix to achieve a dilution factor of 10× and 40× for plasma and urine samples, respectively. Subsequently, 20 μL of diluted plasma sample or 40 μL of diluted urine samples were mixed with 15× volume of acetonitrile-methanol (75:25, v:v) mixture containing 100 ng/ml of ¹³C₄ MMA. Similarly, standards, QCs, and ENQC samples were mixed with a 15× volume of acetonitrile-methanol (75:25, v:v) mixture containing 100 ng/ml of of ¹³C₄ MMA. After vortex mixing these samples were then centrifuged at 12000 g at 4° C. for 15 min. Thirty microliter of the supernatant was then transferred to 96 well plate and dried under stream of N₂ gas at room temperature, then residue reconstituted with 150 μL of acetonitrile-methanol (75:25, v:v) mixture and centrifuged for 5 min at 3220 g at 4° C. The supernatant was injected into LC-MS/MS. The standard curve had a coefficient of determination (R²) value>0.98 in a linear regression. The QC samples had precision and accuracy within ±20% of theoretical values. The peak area ratios of the analyte relative to the IS were used for MMA quantitation.

### Results

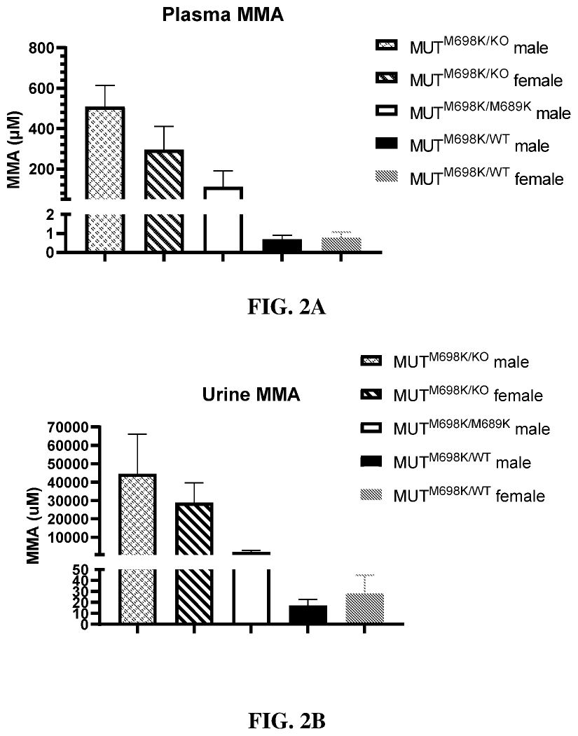

As seen in FIG. 2A, the hypomorphic mutation M698K in methylmalonyl-CoA mutase caused an increase in both plasma methylmalonic acid levels in mice, recapitulating a key disease feature of human methylmalonic acidemia. For example, heterozygous Mut^{M698K/WT} mice had only 0.7-0.8 μM plasma MMA, whereas plasma MMA in homozygous Mut^{M698K/M698K} mice reached 110 μM. Compound heterozygous Mut^{M698K/KO} mice had even higher plasma MMA, from 295 μM (female) to 509 μM (male).

A similar trend was also observed in urine MMA levels (FIG. 2B). For example, heterozygous Mut^{M698K/WT} mice had only 17-28 μM urine MMA, whereas urine MMA in homozygous Mut^{M698K/M698K} mice reached 1,931 μM. Compound heterozygous Mut^{M698}K/KO mice had even higher urine MMA, from 28,806 μM (female) to 44,572 μM (male).

### Example 252. BCAT2^{KO/KO} Survival

### Methods

BCAT2^{KO/KO} mice were divided into three groups. Group one was fed with a regular rodent chow (Prolab® Isopro® RMH 3000) throughout the study. Group two was fed with the regular chow until the mice were 5 weeks old, after which they were provided with free access to both a BCAA-free chow (Dyets, D510081) and a BCAA-containing control chow (Dyets, D510081). This food access was called “low-BCAA diet” in this study. These mice would instinctively eat a balanced ratio of BCAA-free and BCAA-containing food that minimized disease severity (She et al., 2007, Cell Metabolism). Group three was similar to group two, except that the chow was switched when mice were at 3 weeks old. Survival data were imported into Prism to calculate the probability of survival for each group.

### Results

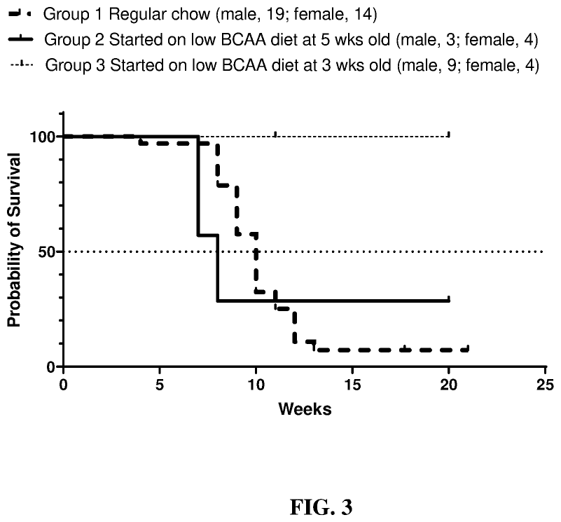

As seen in FIG. 3, feeding BCAT2^{KO/KO} mice with a low-BCAA diet improved postnatal survival. For example, when fed with regular rodent chow (group 1), postnatal death of BCAT2^{KO/KO} mice began at week 4 of life, with the probability of survival gradually decreasing to 7.2% at age 13-week and beyond. When the diet was switched to a low-BCAA diet at 5 weeks old (group 2), postnatal death of BCAT2^{KO/KO} mice began at week 7 of life, with the probability of survival maintaining at 29% from age 8 week and beyond. When the diet was switched to a low-BCAA diet at 3 weeks old (group 3), however, no postnatal death was observed.

### Example 253. MMA Levels in Mut^{M698K/KO} BCAT2^{KO/KO} Mice

### Methods

Mice were fed with normal rodent chow and euthanized at week 5. Terminal blood collection was performed at 8 AM via cardiac puncture into EDTA tubes. Blood was centrifuged for 5 mins. Plasma was collected, snap frozen in liquid nitrogen, and stored at −80° C. until further analysis.

The plasma samples collected from Mut^{M698K/KO} and BCAT2^{KO/KO} mouse model were analyzed for the isoleucine (ILE), methylmalonic acid (MMA), 3-methyl-2-oxo valeric acid (KMV), and (±)-Sodium β-hydroxyisobutyrate (HIB) levels using an LC-MS/MS method. A Waters Acquity UPLC coupled with a SCIEX Triple Quad™ 6500+ mass spectrometer was used for plasma ILE, MMA, and HIB analysis, while an API 4000 triple quadrupole was used for KMV analysis.

ILE, MMA & HIB analysis: Briefly, the test plasma samples were diluted with a surrogate matrix (50% methanol in water) to obtain a 20× for ILE analysis or 10× dilution factor for MMA and HIB analysis. A mixed (ILE and MMA) calibration standard (50-30000 ng/mL) and QCs samples were prepared in the surrogate matrix for ILE and MMA analysis and a similar set of separate calibration standards and QCs were prepared for the plasma HIB analysis only. The ENQC samples were prepared using C₅₇ mice blank plasma samples. The forty microliters of the diluted test samples, calibration standards, QCs, and ENQC samples were quenched with 800 μL of methanol containing 200 ng/ml each of ¹³C₆ isoleucine (¹³C₆ ILE) and ¹³C₄ methylmalonic acid (¹³C₄-MMA) as an internal standard for ILE and MMA, respectively. While for HIB analysis, 40 μL of diluted test samples, calibration standards, QCs, and ENQCs were quenched with 800 μL of methanol containing 200 ng/mL of ¹³C₄ sodium D-3-hydroxybutyrate (¹³C₄—HB) as an internal standard. These samples were vortex mixed and centrifuged at 12000 g at 4° C. for 15 min. An aliquot (100 μL) was withdrawn and dried under the N₂ gas stream at room temperature. The residues were then reconstituted in 70 μL of 0.1% formic acid in water, vortexed for 15 min, and then centrifuged at 3220 g at 4° C. The supernatant was subjected to LC-MS/MS analysis. The chromatographic separation of was carried out using a Waters Acquity UPLC HSS T3 (1.8 m 2.1×150 mm) column maintained at 40° C. and a gradient elution method. Mobile phase A consists of 0.1% formic acid in water and mobile phase B consists of 0.1% formic acid in acetonitrile:methanol (1:1) mixture. For ILE and MMA analysis, the gradient begins at 100% of mobile phase A and continued till 0.2 minutes, then linearly decreased to 93% of mobile phase A till 3.7 min and further decreased to 5% of mobile phase A by 6.7 min and maintained till 7.4 min then immediately increased to 100% of mobile phase A by 7.41 min and maintained till 8.0 min. The retention times for ILE and MMA were 2.99 and 3.18 min, respectively. A polarity switching MRM based MS method using electrospray ionization was employed for ILE (positive ion mode) and MMA (negative ion mode) detection. The compound parameters (ion transitions, declustering potential, and collision energy) for ILE, ¹³C₆-ILE, MMA, and ¹³C₄-MMA are shown in Table 9. General adjustment of the instrument was as follows: Turbo spray temperature was set to 550° C. and nitrogen gas adjustment was set to 55 and 60 units for gas 1 and 2, respectively, curtain gas was set to 40 and the CAD gas was set to 10 units. The ionization voltages were 5500 V (for ILE) and −4500 V (for MMA), and the entrance potentials were 10 V (for ILE) and −10 V (for MMA). In the case of HIB analysis, a slightly modified LC gradient and a separated MS method were used. For HIB analysis, the gradient begins with 97% of mobile phase A and continued till 0.8 min, then linearly decreased to 93% of mobile phase A by 4.2 min and further decreased to 5% of mobile phase A by 6.7 min and maintained till 7.4 min then increased back to 97% of mobile phase A by 7.41 min and maintained till 8.0 min. Similar to MMA, HIB was detected by electrospray ionization in negative ion mode using an MRM based MS method. The compound parameters of HIB and its internal standard are shown in Table 9 and the general adjustment of the instrument was similar to the MMA analysis.

KMV analysis: The test plasma (10 μL) samples were initially diluted with a surrogate matrix to obtain a 3×dilution factor. Additionally, the calibration standards, QCs, and ENQCs samples were prepared in the same manner as described above. Thirty microliters of diluted test samples, calibration standards, QCs, and ENQCs samples were quenched with 120 μL of methanol containing D8-3-methyl-2-oxo valeric acid (200 ng/mL, D₈-KMV) as an internal standard. These samples were vortex mixed and centrifuged at 12000 g at 4° C. for 15 min. An aliquot (100 μL) was dried under the N₂ gas stream at room temperature and residues were then reconstituted with 100 μL of 0.1% formic acid in water, vortexed for 15 min, and then centrifuged at 3220 g at 4° C. The supernatant was subjected to LC-MS/MS analysis. The chromatographic separation was carried out using a Waters Acquity UPLC HSS T3 (1.8 m, 2.1×50 mm) column maintained at 45° C. and a gradient elution method. Mobile phase A consists of 0.1% formic acid and 2 mM ammonium acetate in water: acetonitrile mixture (95:5) and mobile phase B consists of 0.1% formic acid and 2 mM ammonium acetate in acetonitrile: water mixture (95:5). The gradient begins at 95% of mobile phase A and continued till 0.3 minutes, then linearly decreased to 70% of mobile phase A till 2.2 min and further decreased to 5% of mobile phase A by 2.5 min and maintained till 2.8 min then immediately increased to 95% of mobile phase A by 2.81 min and maintained till 3.0 min. The KMV detection was performed by electrospray ionization in negative ion mode using an MRM method. The compound parameters (ion transitions, declustering potential, and collision energy) for KMV and D₈-KMV are shown in Table 9. General adjustment of the instrument was as follows: Turbo spray temperature was set to 550° C., ionization voltage was −4500 V, and nitrogen gas adjustment was set to 55 units for gas 1 and 2. The curtain gas was set to 20 units and the entrance potential was set to −10 V.

TABLE 9
Acquisition parameters and retention times of ILE,
MMA, KMV, HIB, and respective internal standards

| | Q1 | Q3 | Declustering | Collision | Retention |
|---|---|---|---|---|---|
| Analytes | (m/z) | (m/z) | potential (V) | energy (V) | time (min) |
| ILE | 132.1 | 86.2 | 40 | 30 | 2.99 |
| ¹³C₆ ILE | 138.2 | 91.1 | 62 | 15 | 2.98 |
| KMV | 129 | 85 | −35 | −13 | 1.42 |
| D₈-KMV | 137 | 137 | −35 | −5 | 1.39 |
| MMA | 117 | 55 | −35 | −36 | 3.18 |
| ¹³C₄-MMA | 121 | 58.1 | −35 | −34 | 3.18 |
| HIB | 103 | 73 | −35 | −14 | 2.59 |
| ¹³C₄-HB | 107 | 61 | −37 | −16 | 2.38 |

One-way ANOVA with Tukey's multiple comparisons statistical analyses were performed using the Program Prism.

### Results

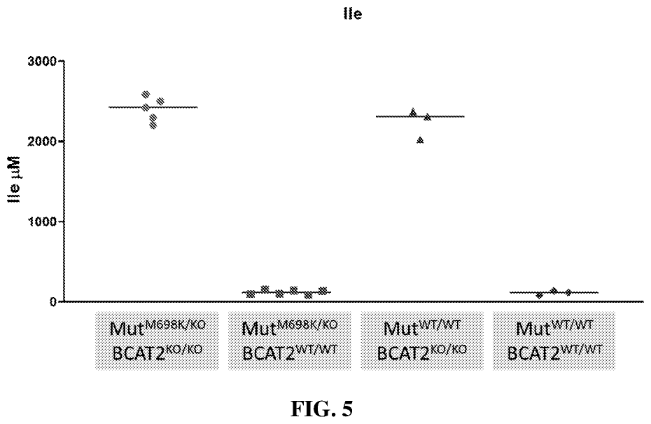

As shown in FIG. 5, plasma isoleucine was only elevated in mice with BCAT2 knocked out, regardless of whether the MMA-CoA mutase (Mut) protein was wildtype (WT) or mutated.

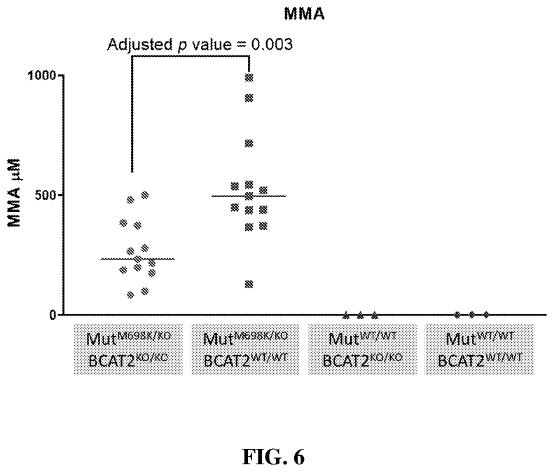

Also, as shown in FIG. 6, mice with WT Mut protein, irrespective of BCAT2 mutation status, had low plasma MMA level (≤1 μM). However, plasma MMA level in Mut^{M698K/KO} mice was significantly reduced when BCAT2 was also knocked out.

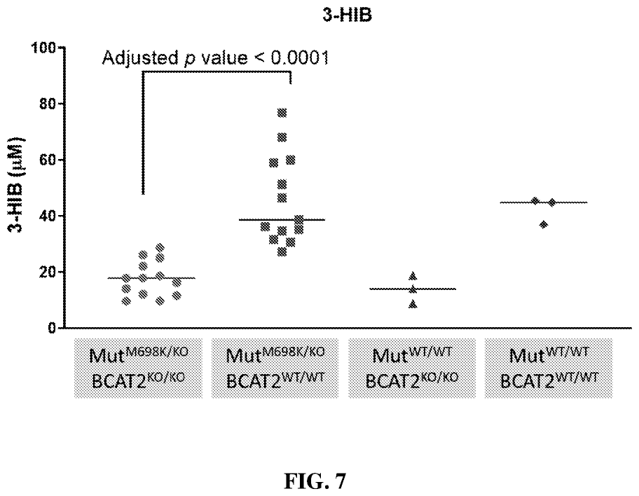

Consistent with the concept that BCAT2 knockout could reduce BCAA catabolic pathway intermediates, 3-HIB, a downstream metabolite of valine breakdown, was also reduced when BCAT2 was knocked out as shown in FIG. 7.

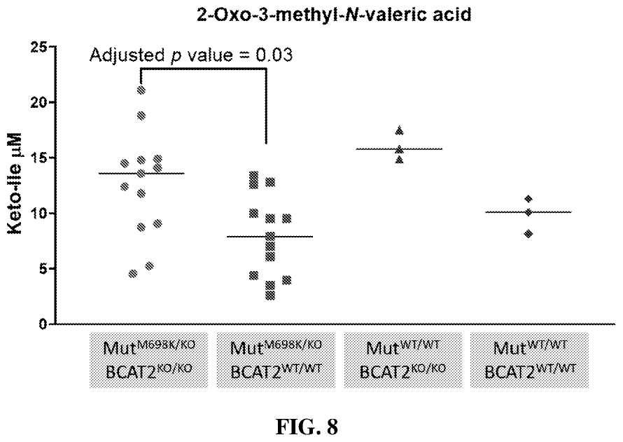

Interestingly, although 2-oxo-3-methyl-N-valeric acid was the direct breakdown product of isoleucine by BCAT2, BCAT2 knockout increased plasma oxo-3-methyl-N-valeric acid level 1.6 to 1.7-fold as shown in FIG. 8.

### Sequences of the Disclosure

### SEQ ID NO: 1

Mouse BCAT2 gene, NCBI Reference Sequence: NM_001412001. The start codon is shown in bold. The adenine at position 245 (exon 3, shown in bold and underlined) is deleted in the BCAT2 KO mouse model, resulting in a stop codon at positions 344-346 (exon 4, shown in bold and underlined). Exon 3 is positions 127-330 of SEQ ID NO: 1. Exon 4 is positions 331-441 of SEQ ID NO: 1.

| SEQ ID NO: 2 | |
|---|---|
| 1 | acagtcacca gccgcacaca ccggatcatg gctgcagcca cactaggaca ggtctgggcc |
| 61 | cgaaagcttc tgcctgttcc ctggcttcta tgtgggtcca aaagatgtgt gtcctccata |
| 121 | ttcaaggctg cagacctcca gattcagatg accaaagaac cacagaagaa gccagcccct |
| 181 | agccaggcac tgctgtttgg gaagaccttc acagaccaca tgctgatggt ggagtggaat |
| 241 | aacaaggctg gatggggccc accaaggatc cagcccttcc agaacctcac gctacaccca |
| 301 | gcctgctctg gtctgcacta ctctctgcag ctctttgagg gcttgaaagc atacaaaggt |
| 361 | ggagaccagc aggtacgcct cttccggccc tggctcaaca tggacaggat gctacgttct |
| 421 | gctaggcgcc tctgcctgcc agactttgac aagcaggagc tgttggagtg tatccgccag |
| 481 | ctcattgaag tagacaaaga ctgggttccc gatggcaacg gaacgagcct ctacgtgcgg |
| 541 | cccgtgctga tcgggaatga gccctcgctg ggtgttggta tggtcacaca agccctcctg |
| 601 | tacgtcattc tgtgccctgt gggctcctac ttccctggag actccatgac acctgtctcc |
| 661 | ctcctggcgg acccttcatt cgtcagagcc tggataggcg gagtcggtga ctgcaagttg |
| 721 | gggggtgtga ccaggctgcc ctccaggaac tatggaccca ctgtggctgt gcagcgagaa |
| 781 | gcccagaaga gaggctgcga gcaggtcctc tggctgtacg ggccagacca ccagctcact |
| 841 | gaggtgggca ccatgaacat ctttgtctac tggacccatg aagacggagt actggagctg |
| 901 | gtaacccccc cactgaatgg cgtcatcttg cctggagtag ttcgacaaag tctgctggat |
| 961 | ctggccagga cttggggtga gttccgggtg gcagaacgca aggtcactat gaaggaattg |
| 1021 | aagcgggcgc tggaggaagg ccgagtgcga gaagtcttcg gctcaggcac tgcttgtcag |
| 1081 | gtctgcccgg tgcaccaaat cctgtatgaa ggcaagcaac tccacatacc taccatggag |
| 1141 | aacggcccag agctcatcct gcgcttccag aaggaactga aggctattca gtacggagcc |
| 1201 | agtgcccacg attggatgtt tcgggtgtga cggtgggggc tgtgccctcc ctggttggat |
| 1261 | ctgcagacgt gccgcatcta gtccagcgtc ctcgcttccc agagactcaa gagaagtgca |
| 1321 | atagggaaag gtctgtccag atgctggatc tctggcaccc cctcgtggac gttacactcc |
| 1381 | aaagccttaa gggxgggacc catgcaattt ggacgccacc tcaaattttg ggtctcagga |
| 1441 | ctcagctgct ccgcgtcctg gtgttcaagg gagaaccttg gcttcttctc tatctccgtt |
| 1501 | ctcaggccgg atccccggtg ctcttcgtgt ccttcccccc cccccccttt tctccgtact |
| 1561 | tgctgcaatt ttgaaataaa atgccaaaga acaaga |

Mouse BCAT2 protein, mitochondrial isoform 4, NCBI Reference Sequence: NP_001398930.1

```
SEQ ID NO: 3
    1 maaatlgqvw arkllpvpwl lcgskrcvss ifkaadlqiq mtkepqkkpa psqallfgkt
  61 ftdhmlmvew nnkagwgppr iqpfqnltlh pacsglhys1 qlfeglkayk ggdqqvrlfr
121 pwlnmdrmlr sarrlclpdf dkqellecir qlievdkdwv pdgngtslyv rpvligneps
181 1gvgmvtqal lyvilcpvgs yfpgdsmtpv slladpsfvr awiggvgdck lggvtrlpsr
241 nygptvavqr eaqkrgceqv lwlygpdhql tevgtmnifv ywthedgvle lvtpplngvi
301 lpgvvrqsll dlartwgefr vaerkvtmke lkraleegrv revfgsgtac qvcpvhqily
361 egkqlhiptm engpelilrf qkelkaiqyg asahdwmfrv
```

Mouse methylmalonyl-Coenzyme A (MMA-CoA) mutase, NCBI Reference Sequence: NM_008650.3. The start codon is shown in bold.

In the MMA-CoA mutase knockout mouse, the guanine (g) at position 311 was changed to adenine (A), resulting in the codon encoding tryptophan (tgg, shown in bold and underlined) being converted to a stop codon (tag). Exon 2 is positions 96 to 514 of SEQ ID NO: 3.

In the MMA-CoA mutase M698K mutant, the thymine (T) at position 2228 (methionine codon ATG, shown in bold and underlined) was changed to adenine (A), resulting in a change from methionine to lysine (AAG) in the corresponding protein.

| SEQ ID NO: 4 | |
|---|---|
| 1 | gattggctgc cgagctcctg cggcgagcgg gtgtgacccc ggcgtctggg ttcggttctg |
| 61 | aagtccgggc ttggtccggg tgatccccaa acactgaccg ttctcatttc cttttgggag |
| 121 | tattcatgct tcaccatgtt gagagctaag aatcaacttt ttttgctatc gccccattac |
| 181 | ctgaagcagc taaacattcc atcagcttcc agatggaaac gccttctaca ccagcagcaa |
| 241 | ccccttcacc cagaatgggc tgtactggcc aaaaagcagc tgaaaggcaa aaacccagag |
| 301 | gaccttatat ggcacacccc agaagggatc tctataaagc ccttatattc cagggcagat |
| 361 | actctggact tacccgaaga acttccagga gtgaagccat tcacacgggg accatatcct |
| 421 | accatgtata cctataggcc ctggaccatc cgtcagtatg caggctttag tactgtggaa |
| 481 | gaaagcaata aattctataa ggacaatatt aaggctggtc agcaggggtt gtctgttgcc |
| 541 | tttgacttgg caacacatcg tggttatgat tcagacaacc ccagagttcg tggagatgtt |
| 601 | ggaatggctg gagttgctat tgacactgta gaagacacca aaatcctatt tgatggcatt |
| 661 | cctttagaaa aaatgtccgt ttccatgact atgaacggag ctgtcatccc agtcctggca |
| 721 | acatttatag taactgggga agagcaaggt gtgccgaagg agaagctcac tggcacaatt |
| 781 | cagaatgaca tcctaaagga gtttatggtc agaaatactt atatttttcc cccagagcca |
| 841 | tcgatgaaaa ttattgctga cattttccaa tacacagcac agcacatgcc aaaatttaat |
| 901 | tccatttcga ttagcgggta ccatatgcag gaagcaggag ctgatgccat tttagaactg |
| 961 | gcctatacca tcgcagatgg gttggagtac tgcagaactg gactccaggc tggactcaca |
| 1021 | attgatgaat ttgcaccaag gttgtctttc ttctggggaa ttggaatgaa cttctacatg |
| 1081 | gaaatagcca agatgcgagc tgggagaaga ctgtgggctc acttaataga aaaaatgttc |
| 1141 | cagcctaaaa actctaaatc tcttcttcta agagcacact gccagacatc ggggtggtca |
| 1201 | cttactgagc aggatcctta caataacatt gtccgcactg caatcgaagc catggcagct |
| 1261 | gtgtttggag gaacccagtc tttgcatacg aactcttttg atgaagcgct gggtttgccc |
| 1321 | actgtgaaaa gtgcccggat tgctcggaac acacagatca tcattcagga agaatctggg |
| 1381 | atccccaaag tggcggatcc ttggggaggg tcgtacatga tggagtcgct cacaaatgac |
| 1441 | gtttatgagg ctgctctgaa gttgatatat gaagttgaag aaatgggtgg aatggccaaa |
| 1501 | gctgtagctg aaggaatccc taaacttcgc attgaagaat gtgctgcccg aagacaagct |
| 1561 | agaatagatt ctggttctga ggtaattgtt ggagtaaata agtatcagtt ggaaaaagaa |
| 1621 | gactctgtgg aggtcctggc cattgacaac acttcagtgc gtaagaagca gattgaaaaa |
| 1681 | ctcaagaaga ttaaatccag cagggatcaa gctttggctg agcagtgtct cagtgcactt |
| 1741 | acccagtgtg ctgccagtgg agatggcaat attctggctc tggcagtgga tgcagctcgt |
| 1801 | gcaagatgta cagttggaga aatcacggat gccttgaaaa aagtatttgg tgagcataaa |
| 1861 | gctaatgatc gtatggtgag tggagcatat cggcaggagt ttggagaaag taaagagatc |
| 1921 | acatctgcca tcaagagagt taataaattc atggaacgtg aaggtcgcag acctcgtctt |
| 1981 | cttgtggcaa aaatgggaca agatggccat gacaggggag ccaaggtcat tgctacagga |
| 2041 | tttgctgatc ttggttttga tgtggacata ggccctcttt ttcagactcc ccgtgaagtg |
| 2101 | gcgcagcaag ctgtggatgc agatgtccat gctgtgggtg tcagcacact tgctgctggt |
| 2161 | cataaaaccc tcgttcctga gcttatcaaa gaactcaccg ccctggggcg gccagatatc |
| 2221 | cttgtcatgt gtgggggcgt gattccacca caggattatg aatttctgta tgaagttggt |
| 2281 | gtttccaacg tctttggtcc tggaacccgg attccaagag ctgctgtcca agtgcttgat |
| 2341 | gatattgaga agtgtttggc agagaagcag cagtctgtgt aacatctgga tctttagtta |
| 2401 | tgactgaagc agcagtctgt gtaacatctg gatctttagt tatgaccgaa gaaggtagtg |
| 2461 | gtaaatccgt ttcgtcaggt aatcccagca gctcttagaa tgcttgcttt ggaaacctca |
| 2521 | cattaaaaga ttctaactat aaatgtgcag tcatgccaat acacatattt aaagaatctg |
| 2581 | agttcaaaaa aaaaaaaaaa acctctgaaa atcatttata ccaaatatgt tttaagtata |
| 2641 | ttattctaga aaattaaaat ggtatactat ttagaaatag cttacaattc ttataaataa |
| 2701 | ttgaactttt gttttgtttt atcctttgac atggggtctt actatgtagc actggttgtc |
| 2761 | ttgcaactaa ctctgtagaa cagtctggcc tcaaactcac agagatccac ctgcctctgc |
| 2821 | cttcctagta cagggattaa aggtatgtgc ctccatgtcc tgcaaattat taaacttaaa |
| 2881 | aattaaataa aattttaaaa tttaaccgat tcttccttca atttagtgtt gatttgtgta |
| 2941 | ctagaaactc aagttcctgt gttaatttaa taaaataatg acatttatct tacaagaaat |
| 3001 | agtggagggt atttatggat ggatcctcag ccagacctac tattgcttca ataaaagtag |
| 3061 | tgtttatcac cccctgtaca atggaatagg ggacatggta gataggcctt ccaggattgt |
| 3121 | gtcagctcct cacatgggaa tgatattgtt aatgtcaggt tcaaaactct ggaaaagatg |
| 3181 | tgtgtgacgc tctcttaaat gaatgttgat ggacagatgc atttgctata atgacagtct |
| 3241 | aaatctatta cataaagtaa atgaattttc agatctagaa acacgctgat ctgtatacac |
| 3301 | tttcttatta atataagtta taaaaataaa aaaatagtct gaagaactct tgtagtgtga |
| 3361 | tgttacttca ctcattgaag gaagagactc actctgtgtt aacctaatgt attcagcgaa |
| 3421 | ggtgaggtga gcaacaagga ttcttttgta actttctaca ttatagatca aataatattg |
| 3481 | gggagggaaa atcatataat gctgtaaaat taataaacta tactatagtt taatttttag |
| 3541 | accatgactc ttaggagggc atcatgatta ttgttttcta ttttaaagat gagcagtttg |
| 3601 | acgtcaagga caacaatata tttattgtgt tatcttatgt actacttata aagaaatata |
| 3661 | tatttgataa atacg |

Mouse methylmalonyl-Coenzyme A mutase. The tryptophan (W) at position 59 (shown in bold and underlined) was changed to a stop codon to generate the MMA-CoA knockout. The methionine (M) at position 698 is also shown in bold and underlined.

SEQ ID NO: 5
MLRAKNQLFLLSPHYLKQLNIPSASRWKRLLHQQQPLHPEWAVLAKKQLK
GKNPEDLIWHTPEGISIKPLYSRADTLDLPEELPGVKPFTRGPYPTMYTY
RPWTIRQYAGFSTVEESNKFYKDNIKAGQQGLSVAFDLATHRGYDSDNPR
VRGDVGMAGVAIDTVEDTKILFDGIPLEKMSVSMTMNGAVIPVLATFIVT
GEEQGVPKEKLIGTIQNDILKEFMVRNTYIFPPEPSMKIIADIFQYTAQH
MPKENSISISGYHMQEAGADAILELAYTIADGLEYCRTGLQAGLTIDEFA
PRLSFFWGIGMNFYMEIAKMRAGRRLWAHLIEKMFQPKNSKSLLLRAHCQ
TSGWSLTEQDPYNNIVRAIEAMAAVFGGTQSLHTNSFDEALGLPTVKSAR
IARNTQIIIQEESGIPKVADPWGGSYMMESLINDVYEAALKLIYEVEEMG
GMAKAVAEGIPKLRIEECAARRQARIDSGSEVIVGVNKYQLEKEDSVEVL
AIDNTSVRKKQIEKLKKIKSSRDQALAEQCLSALTQCAASGDGNILALAV
DAARARCTVGEITDALKKVFGEHKANDRMVSGAYRQEFGESKEITSAIKR
VNKFMEREGRRPRLLVAKMGQDGHDRGAKVIATGFADLGFDVDIGPLFQT
PREVAQQAVDADVHAVGVSTLAAGHKTLVPELIKELTALGRPDILVMCGG
VIPPQDYEFLYEVGVSNVFGPGTRIPRAAVQVLDDIEKCLAEKQQSV

Mouse propionyl-Coenzyme A carboxylase gene. The start codon is shown in bold.

Exon 5 is nucleotides 328-441 of SEQ ID NO: 5. The KO is introduced by adding an additional adenine (A) after the adenine (A) at position 379 (shown in bold) of SEQ ID NO: 5. This addition creates a premature stop codon at positions 381-383 (shown in bold and underlined) of SEQ ID NO: 5.

The A134T substitution is introduced by changing the guanine (G) at position 439 (shown in bold and underlined) to an adenine (A). This substitution changes the alanine (A) codon GCT to the threonine (T) codon ACT.

| SEQ ID NO: 6 | |
|---|---|
| 1 | ggtgagaggt cagcagccgg caagtctgcg gggtggacga tggcggggca gtgggtcagg |
| 61 | accgtggcgc tgttggcggc caggcggcat tggcggcggt cctcgcagca gcaattgctg |
| 121 | gggacgctga agcatgctcc agtctattca taccaatgcc tagtggtgtc cagaagtctc |
| 181 | agttctgtgg aatatgagcc taaagaaaag acttttgata aaattctcat tgctaacaga |
| 241 | ggagaaattg cctgtagggt tattaaaact tgcaagaaga tgggcatcaa gacagttgcc |
| 301 | attcacagtg atgttgatgc cagttctgtt cacgtgaaaa tggcggatga ggctgtctgt |
| 361 | gttggcccag ctcccaccag taaaagctac ctcaacatgg atgccatcat ggaagccatt |
| 421 | aagaaaacca gggcccaagc tgtacaccca gggtatggat tcctgtcaga aaacaaagag |
| 481 | tttgcaaagc gtctggcagc agaagatgtc actttcattg gacctgatac tcatgctatt |
| 541 | caagccatgg gtgacaagat agaaagcaaa ctattagcca agagagcaaa ggtcaacaca |
| 601 | atccctggtt ttgatggggt agtaaaggat gcagatgaag ctgtcagaat tgcaagggaa |
| 661 | attggctacc ctgtgatgat caaggcctca gcaggcggtg gtgggaaagg catgcgcatc |
| 721 | gcctgggatg acgaagagac cagggatggc tttagatttt catcccagga agctgcttct |
| 781 | agttttggtg atgatagact actaatagaa aaatttatcg ataaccctcg tcatatagaa |
| 841 | atccaggttt taggggataa acatggcaat gctctgtggc tcaatgagag ggagtgctcg |
| 901 | atccagagaa ggaaccagaa ggtggtagag gaggcgccaa gcatttttct ggatcctgaa |
| 961 | actcgccaag caatgggaga gcaggccgtg gctttggcta aagccgtgaa gtattcttct |
| 1021 | gctggaactg tggaatttct tgtggactcc cagaagaatt tttacttctt ggagatgaat |
| 1081 | acaagactac aggtcgaaca tcctgtcaca gagtgcatta ctggcctgga cttagtccaa |
| 1141 | gaaatgatcc ttgttgctaa gggttaccca ctcaggcaca agcaagagga tattcccatc |
| 1201 | agtggctggg cagttgaatg tcgggtttat gctgaggacc cctacaagtc tttcggttta |
| 1261 | ccgtctattg ggaggctgtc ccagtaccaa gagccgatac atctacctgg tgtccgagtt |
| 1321 | gacagtggca tccaaccagg aagtgacatc agcatctatt atgatcctat gatttcaaag |
| 1381 | ctagtcacat atgggtctga cagagcagaa gccctgaaga ggatggaaga cgcactggac |
| 1441 | aattatgtga tccggggtgt tacacacaac atcccattgc tccgggaggt gataatcaac |
| 1501 | acacgttttg tgaaaggaga catcagcact aagtttctct ctgatgtgta tcctgatggc |
| 1561 | ttcaaagggc acacgttaac actgagtgag agaaaccagt tattggccat tgcatcatct |
| 1621 | gtatttgtgg catcccagct acgagctcag cgcttccaag aacattcaag agtaccagtt |
| 1681 | attaggcctg atgtggctaa gtgggagctc tcggtaaagt tacatgatga agatcatact |
| 1741 | gtcgtggcat ctaacaatgg gccggcattt accgtggaag ttgatggctc gaaactaaat |
| 1801 | gtgaccagta cgtggaacct ggcgtcaccc ttattgtctg tcaacgttga tggcacgcag |
| 1861 | aggactgtgc agtgtctttc tcgggaagca ggtggaaaca tgagcatcca gtttcttggc |
| 1921 | acagtgtaca aagtgcacat tttaaccaag cttgctgcag agctgaacaa attcatgctt |
| 1981 | gaaaaagtgc ccaaggacac cagcagcact ctgtgctccc cgatgcctgg agtggtggtg |
| 2041 | gccgtttctg tcaagcctgg agacatggta gcagaaggtc aggaaatctg tgtgattgaa |
| 2101 | gctatgaaaa tgcagaacag tatgacagct gggaaaatgg gcaaggtgaa attggtgcac |
| 2161 | tgcaaagctg gagacacagt tggtgaagga gacctgcttg tggagctgga atgaagtatt |
| 2221 | tctagccttt ccgtcagcac tgctcactgc tttaactggc gtctccatta ttctctaacc |
| 2281 | ctcaatttgc gcaagcattt acaggagcac tcctgtgcag gaggttctac agcctcctgt |
| 2341 | ttattcctca gaagaggcaa aaccgacatt ctgccaacaa atcaccaatg gaaattcttg |
| 2401 | atataaatac ttgtacatat gatttgtact tctgctgaga tttcctagtg tcaaaattaa |
| 2461 | atcaataaaa ccaagcattt gtctaaatat tagtttgccc ttttttccga atgaagacaa |
| 2521 | tatacacaga ggcgacaggc tgtgccaatt gactgtaaat attagcattt cattgtgatt |
| 2581 | ctttaaaaat ttatataaat tcc |

Mouse propionyl-Coenzyme A carboxylase protein. The position of the A134T substitution is shown in bold and underlined.

MAGQWVRIVALLAARRHWRRSSQQQLLGTLKHAPVYSYQCLVVSRSLSSV
EYEPKEKTEDKILIANRGEIACRVIKTCKKMGIKTVAIHSDVDASSVHVK
MADEAVCVGPAPTSKSYLNMDAIMEAIKKTRAQAVHPGYGFLSENKEFAK
RLAAEDVTFIGPDTHAIQAMGDKIESKLLAKRAKVNTIPGFDGVVKDADE
AVRIAREIGYPVMIKASAGGGGKGMRIAWDDEETRDGFRFSSQEAASSFG
DDRLLIEKFIDNPRHIEIQVLGDKHGNALWLNERECSIQRRNQKVVEEAP
SIFLDPETRQAMGEQAVALAKAVKYSSAGTVEFLVDSQKNFYFLEMNTRL
QVEHPVTECITGLDLVQEMILVAKGYPLRHKQEDIPISGWAVECRVYAED
PYKSFGLPSIGRLSQYQEPIHLPGVRVDSGIQPGSDISIYYDPMISKLVT
YGSDRAEALKRMEDALDNYVIRGVTHNIPLLREVIINTRFVKGDISTKFL
SDVYPDGFKGHTLTLSERNQLLAIASSVEVASQLRAQRFQEHSRVPVIRP
DVAKWELSVKLHDEDHTVVASNNGPAFTVEVDGSKLNVTSTWNLASPLLS
VNVDGTQRTVQCLSREAGGNMSIQFLGTVYKVHILTKLAAELNKFMLEKV
PKDTSSTLCSPMPGVVVAVSVKPGDMVAEGQEICVIEAMKMQNSMTAGKM
GKVKLVHCKAGDTVGEGDLLVELE

What is claimed:

1. A compound of Formula I or II: 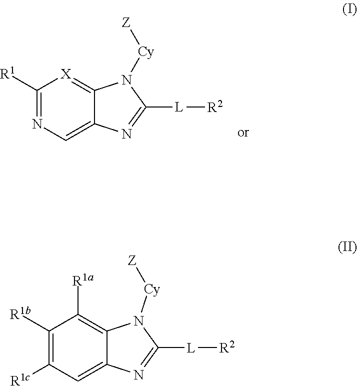
or a pharmaceutically acceptable salt thereof, wherein:
R¹ is optionally substituted heteroaryl, optionally substituted aryl, or optionally substituted 4-, 5- or 6-membered heterocycloalkyl;
R^{1a} is hydrogen or fluoro;
R^{1b} is optionally substituted heteroaryl, optionally substituted aryl, or optionally substituted 4-, 5- or 6-membered heterocycloalkyl;
R^{1c} is hydrogen, halo, C₁-C₆alkyl, C₁-C₆haloalkyl, C₁-C₆alkoxy, or C₁-C₆haloalkoxy;
X is CH or N;
Cy is optionally substituted C₅-C₇ cycloalkyl or optionally substituted 6- or 7-membered heterocycloalkyl;
Z is —NHC(O)Ar¹, 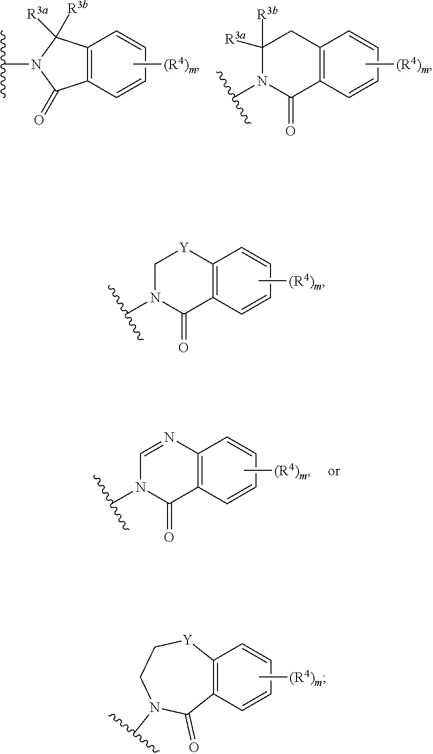
L is a bond, —O—, —NR^{a}—, or optionally substituted C₁-C₄alkylene;
R^{a} is hydrogen or C₁-C₄alkyl;
R² is optionally substituted phenyl, optionally substituted pyridinyl, optionally substituted 5-membered heteroaryl, optionally substituted 4-, 5-, or 6-membered heterocycloalkyl, optionally substituted C₁-C₆alkyl, optionally substituted C₁-C₆alkoxy, optionally substituted C₁-C₆haloalkyl, optionally substituted C₁-C₆haloalkoxy, optionally substituted C₃-C₆cycloalkyl, or NR^{b}R^{c};
R^{b} and R^{c} are each independently hydrogen, C₁-C₆alkyl, C₁-C₆haloalkyl, OH, C₁-C₄alkoxy, C₁-C₆hydroxyalkyl, or C₃-C₆cycloalkyl;
Ar¹ is optionally substituted 6-membered heteroaryl, optionally substituted phenyl, or 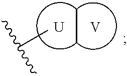
U is an optionally substituted phenyl or optionally substituted 6-membered heteroaryl;
V is an optionally substituted phenyl, optionally substituted 5- or 6-membered heterocycloalkyl, or optionally substituted 5- or 6-membered heteroaryl; and
wherein the point of attachment to the carbonyl group of —NHC(O) for variable Z occurs at the optionally substituted phenyl or optionally substituted 6-membered heteroaryl of U;
Y is O or NH;
R^{3a} is hydrogen, C₁-C₃alkyl, or oxo;
R^{3b} is hydrogen or C₁-C₃alkyl; or alternatively, R^{3a} and R^{3b} are taken together to form cyclopropyl;
each R⁴ is independently halo, cyano, C₁-C₆alkyl, C₁-C₆haloalkyl, or C₃-C₆cycloalkyl; and
m is 0, 1, 2, 3, or 4.

2. The compound according to claim 1, wherein the compound is a compound of Formula I, or a pharmaceutically acceptable salt thereof.

3. The compound according to claim 2, wherein the compound of Formula I is a compound of Formula I-A: 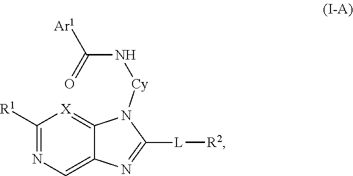
or a pharmaceutically acceptable salt thereof.

4. The compound according to claim 2, wherein the compound of Formula I is a compound Formula I-B or Formula I-C: 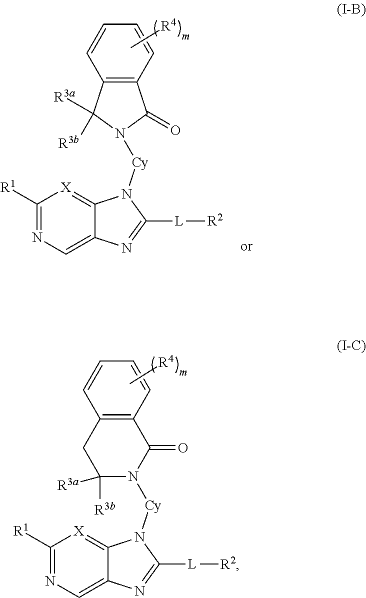
or a pharmaceutically acceptable salt thereof.

5. The compounds according to claim 4, or a pharmaceutically acceptable salt thereof, wherein the compound of Formula I is a compound of Formula I-B.

6. The compounds according to claim 4, or a pharmaceutically acceptable salt thereof, wherein the compound of Formula I is a compound of Formula I-C.

7. The compound according to claim 2, wherein the compound of Formula I is a compound Formula I-D, Formula I-E, or Formula I-F: 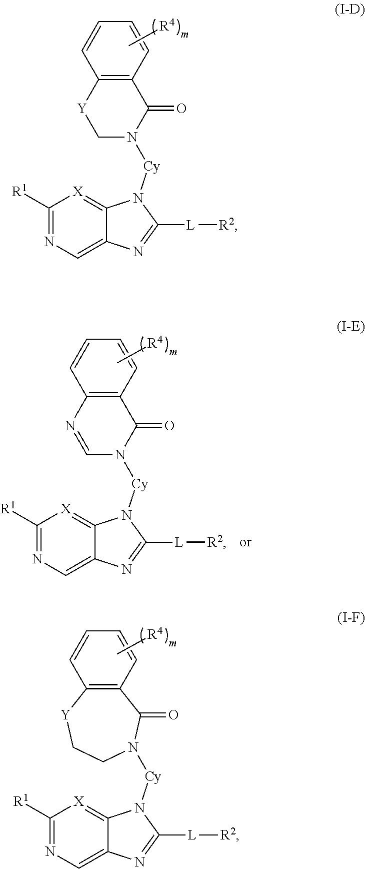
or a pharmaceutically acceptable salt thereof.

8. The compounds according to claim 7, or a pharmaceutically acceptable salt thereof, wherein the compound of Formula I is a compound of Formula I-D.

9. The compounds according to claim 7, or a pharmaceutically acceptable salt thereof, wherein the compound of Formula I is a compound of Formula I-E.

10. The compounds according to claim 7, or a pharmaceutically acceptable salt thereof, wherein the compound of Formula I is a compound of Formula I-F.

11. The compound according to claim 1, wherein the compound is a compound of Formula II, or a pharmaceutically acceptable salt thereof.

12. The compound according to claim 11, wherein the compound of Formula II is a compound of Formula II-A: 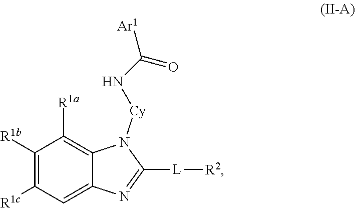
or a pharmaceutically acceptable salt thereof.

13. The compound according to claim 11, wherein the compound of Formula II is a compound of Formula II-B or Formula II-C: 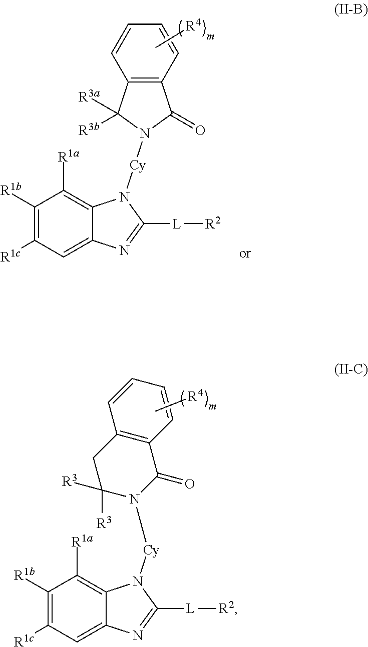
or a pharmaceutically acceptable salt thereof.

14. The compound according to claim 13, or a pharmaceutically acceptable salt thereof, wherein the compound of Formula II is a compound of Formula II-B.

15. The compound according to claim 13, or a pharmaceutically acceptable salt thereof, wherein the compound of Formula II is a compound of Formula II-C.

16. The compound according to claim 11, wherein the compound of Formula II is a compound of Formula II-D, Formula II-E, or Formula II-F: 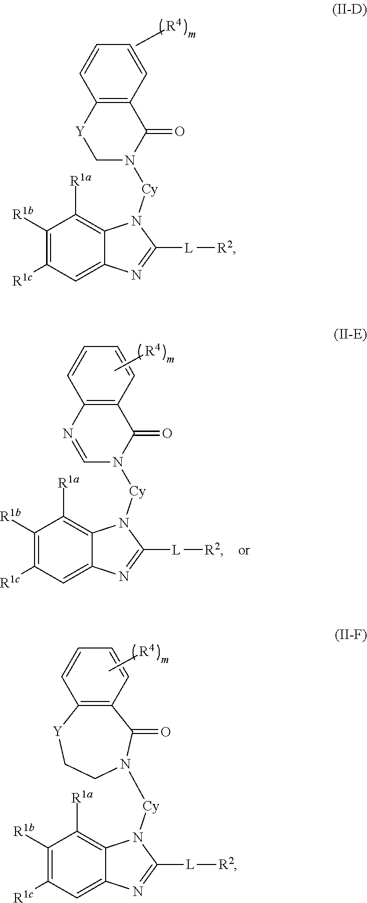
or a pharmaceutically acceptable salt thereof.

17. The compound according to claim 16, or a pharmaceutically acceptable salt thereof, wherein the compound of Formula II is a compound of Formula II-D.

18. The compound according to claim 16, or a pharmaceutically acceptable salt thereof, wherein the compound of Formula II is a compound of Formula II-E.

19. The compound according to claim 16, or a pharmaceutically acceptable salt thereof, wherein the compound of Formula II is a compound of Formula II-F.

20. The compound according to any one of claims 1-10, or a pharmaceutically acceptable salt thereof, wherein X is CH.

21. The compound according to any one of claims 1-10, or a pharmaceutically acceptable salt thereof, wherein X is N.

22. The compound according to any one of claims 1, 2, 7, 8, 10, 11, 16, 17, or 19-21, or a pharmaceutically acceptable salt thereof, wherein Y is O.

23. The compound according to any one of claims 1, 2, 7, 8, 10, 11, 16, 17, or 19-21, or a pharmaceutically acceptable salt thereof, wherein Y is NH.

24. The compound according to any one of claims 1, 2, 4-6, 11, 13-15, 20, or 21, or a pharmaceutically acceptable salt thereof, wherein R^{3a} and R^{3b} are hydrogen.

25. The compound according to any one of claims 1, 2, 4-6, 11, 13-15, 20, or 21, or a pharmaceutically acceptable salt thereof, wherein R^{3a} is oxo and R^{3b} is hydrogen.

26. The compound according to any one of claims 1, 2, 4-11, or 13-25, or a pharmaceutically acceptable salt thereof, wherein each R⁴ is independently halo.

27. The compound according to claim 26, or a pharmaceutically acceptable salt thereof, wherein each R⁴ is independently F or Cl.

28. The compound according to any one of claims 1, 2, 4-11, or 13-27, or a pharmaceutically acceptable salt thereof, wherein m is 1 or 2.

29. The compound according to any one of claims 1-28, or a pharmaceutically acceptable salt thereof, wherein Cy is optionally substituted C₅-C₇ cycloalkyl.

30. The compound according to claim 29, or a pharmaceutically acceptable salt thereof, wherein Cy is unsubstituted C₅-C₇ cycloalkyl.

31. The compound according to claim 29, or a pharmaceutically acceptable salt thereof, wherein Cy is C₅-C₇ cycloalkyl substituted with one or more of OH, deuterium, halo, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), 0C₀₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}, wherein R^{d} is hydrogen or C₁-C₄alkyl and R^{e} is hydrogen, C₁-C₄alkyl, CO₂(C₁-C₄alkyl), C(O)(C₁-C₄alkyl), SO₂(C₁-C₄alkyl), or SO₂(C₃-C₄cycloalkyl).

32. The compound according to claim 29, or a pharmaceutically acceptable salt thereof, wherein the optionally substituted C₅-C₇cycloalkyl is an optionally substituted cyclohexyl or an optionally substituted cycloheptyl.

33. The compound according to claim 32, or a pharmaceutically acceptable salt thereof, wherein the optionally substituted cyclohexyl is

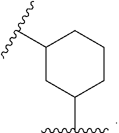 [SMILES: *C1CCCC(*)C1]

34. The compound according to claim 33, or a pharmaceutically acceptable salt thereof, wherein the optionally substituted cyclohexyl is

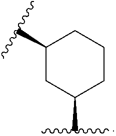 [SMILES: *[C@@H]1CCC[C@H](*)C1]

35. The compound according to claim 32, or a pharmaceutically acceptable salt thereof, wherein the optionally substituted cyclohexyl is substituted with one of OH, C₁-C₃alkoxy, C₁-C₃haloalkoxy or NR^{d}R^{e}, wherein R^{d} is hydrogen or C₁-C₄alkyl and R^{e} is hydrogen or C₁-C₄alkyl.

36. The compound according to claim 32, or a pharmaceutically acceptable salt thereof, wherein the optionally substituted cyclohexyl is

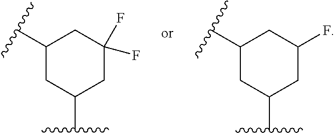 [SMILES: *C1CC(*)CC(F)(F)C1 ; *C1CC(*)CC(F)C1]

37. The compound according to claim 36, or a pharmaceutically acceptable salt thereof, wherein the optionally substituted cyclohexyl is

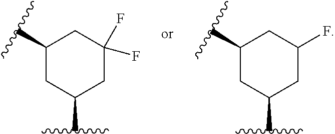 [SMILES: *[C@@H]1C[C@H](*)CC(F)(F)C1 ; *[C@@H]1CC(F)C[C@H](*)C1]

38. The compound according to claim 32, or a pharmaceutically acceptable salt thereof, wherein the optionally substituted cycloheptyl is

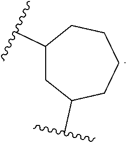 [SMILES: *C1CCCCC(*)C1]

39. The compound according to claim 38, or a pharmaceutically acceptable salt thereof, wherein the optionally substituted cycloheptyl is

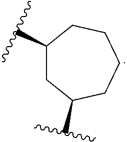 [SMILES: *[C@@H]1CCCC[C@H](*)C1]

40. The compound according to claim 32, or a pharmaceutically acceptable salt thereof, wherein the optionally substituted cycloheptyl is substituted with one of OH or C₁-C₃alkoxy.

41. The compound according to any one of claims 1-28, or a pharmaceutically acceptable salt thereof, wherein Cy is optionally substituted 6-membered heterocycloalkyl.

42. The compound according to claim 41, or a pharmaceutically acceptable salt thereof, wherein Cy is optionally substituted piperidinyl, optionally substituted tetrahydropyranyl, optionally substituted thianyl, optionally substituted thianyl sulfone, optionally substituted thianyl sulfoxide, or optionally substituted 1-oxo-1-imino-1-thiacyclohexyl.

43. The compound according to claim 41 or 42, or a pharmaceutically acceptable salt thereof, wherein Cy is optionally substituted piperidinyl.

44. The compound according to claim 43, or a pharmaceutically acceptable salt thereof, wherein the nitrogen in the piperidinyl is substituted with C₁-C₆alkyl, C(O)(C₁-C₆alkyl), oxo, OH, C₁-C₆alkoxy, CO₂(C₁-C₆alkyl), SO₂(C₁-C₆alkyl), or SO₂(C₃-C₆cycloalkyl).

45. The compound according to claim 44, or a pharmaceutically acceptable salt thereof, wherein the nitrogen in the piperidinyl is substituted with C₁-C₄alkyl or C(O)(C₁-C₄alkyl).

46. The compound according to any one of claims 1-28, or a pharmaceutically acceptable salt thereof, wherein Cy is optionally substituted 7-membered heterocycloalkyl, such as optionally substituted oxazepanyl, optionally substituted thiepanyl, optionally substituted thiepanyl-1-oxide, optionally substituted thiepanyl-1,1-dioxide, optionally substituted azepanyl, or optionally substituted 1-imino-1-oxide-thiepanyl.

47. The compound according to any one of claim 1 or 2, wherein the compound of Formula I is a compound of Formula I-G: 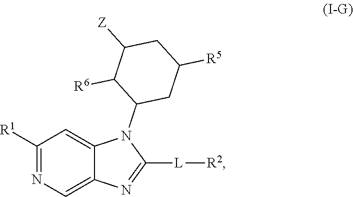
or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), 0C₀₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}; and
the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl).

48. The compound according to claim 47, wherein the compound of Formula I-G is a compound of Formula I-G-1: 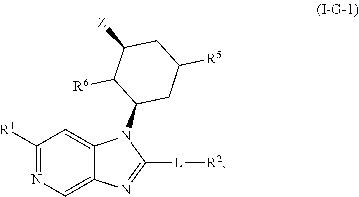
or a pharmaceutically acceptable salt thereof.

49. The compound according to any one of claim 1 or 11, wherein the compound of Formula II is a compound of Formula II-G: 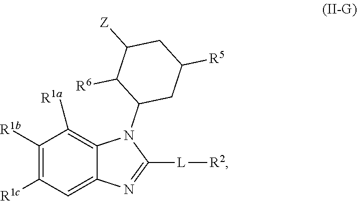
or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), 0C₀₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e}; and
the other of R⁵ or R⁶ is hydrogen or deuterium; and
wherein R^{d} is hydrogen or C₁-C₄alkyl and R^{e} is hydrogen, C₁-C₄alkyl, C₀₂(C₁-C₄alkyl), C(O)C₁-C₄alkyl, SO₂(C₁-C₄alkyl), or SO₂(C₃-C₄cycloalkyl).

50. The compound according to claim 49, wherein the compound of Formula II-G is a compound of Formula II-G-1: 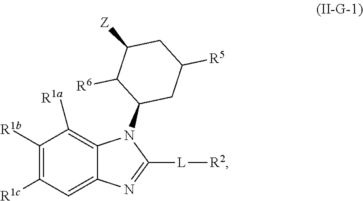
or a pharmaceutically acceptable salt thereof.

51. The compound according to any one of claims 47-50, or a pharmaceutically acceptable salt thereof, wherein R⁵ is OH, C₁-C₃alkoxy, C₁-C₃haloalkoxy, or NH₂, and R⁶ is hydrogen.

52. The compound according to any one of claims 47-50, or a pharmaceutically acceptable salt thereof, wherein R⁵ is hydrogen and R⁶ is OH, C₁-C₃alkoxy, C₁-C₃haloalkoxy, or NH₂.

53. The compound according to claim 51 or 52, or a pharmaceutically acceptable salt thereof, wherein the C₁-C₃alkoxy is OCH₃ and the C₁-C₃haloalkoxy is OCF₃ or OCHF₂.

54. The compound according to any one of claim 1 or 2, wherein the compound of Formula I is a compound of Formula I-H or Formula I-J: 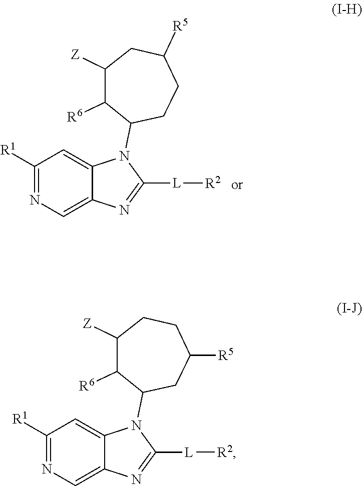
or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), OCO₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e};
and the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl).

55. The compound according to claim 54, wherein the compound of Formula I-H is a compound of Formula I-H-1, or a pharmaceutically acceptable salt thereof, and the compound of Formula I-J is a compound of Formula I-J-1, or a pharmaceutically acceptable salt thereof:

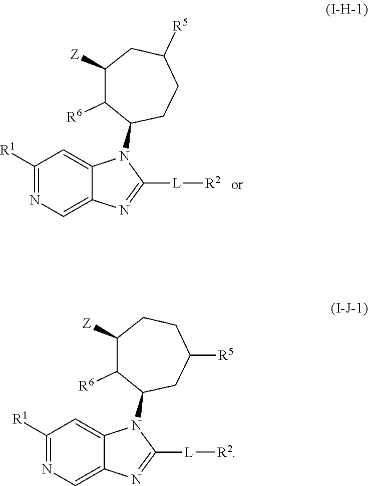

56. The compound according to any one of claim 1 or 11, wherein the compound of Formula II is a compound of Formula II-H or Formula II-J: 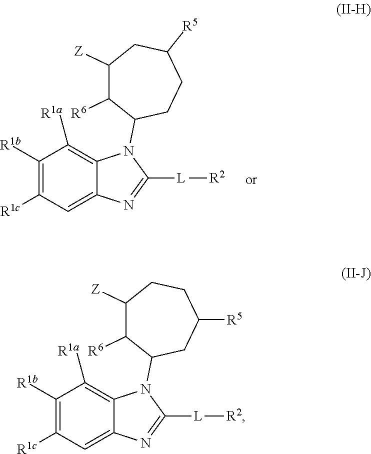
or a pharmaceutically acceptable salt thereof, wherein:
one of R⁵ or R⁶ is OH, cyano, C₁-C₄alkyl, C₁-C₄haloalkyl, C₁-C₄alkoxy, deuterated C₁-C₄alkoxy, C₁-C₄haloalkoxy, OCO₂(C₁-C₄alkyl), 0C₀₂(C₃-C₄cycloalkyl), OSO₂(C₁-C₄alkyl), OSO₂(C₃-C₄cycloalkyl), OC(O)(C₁-C₄alkyl), OC(O)(C₃-C₄cycloalkyl), N₃, or NR^{d}R^{e};
and the other of R⁵ or R⁶ is hydrogen or deuterium; and
wherein R^{d} is hydrogen or C₁-C₄alkyl and R^{e} is hydrogen, C₁-C₄alkyl, CO₂(C₁-C₄alkyl), C(O)C₁-C₄alkyl, SO₂(C₁-C₄alkyl), or SO₂(C₃-C₄cycloalkyl).

57. The compound according to claim 56, wherein the compound of Formula II-H is a compound of Formula II-H-1, or a pharmaceutically acceptable salt thereof, and the compound of Formula II-J is a compound of Formula II-J-1, or a pharmaceutically acceptable salt thereof:

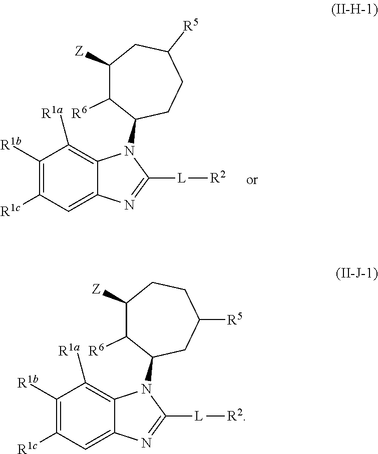

58. The compound according to any one of claims 54-57, or a pharmaceutically acceptable salt thereof, wherein R⁵ is OH or C₁-C₃alkoxy and R⁶ is hydrogen.

59. The compound according to any one of claims 54-57, or a pharmaceutically acceptable salt thereof, wherein R⁵ is hydrogen and R⁶ is OH or C₁-C₃alkoxy.

60. The compound according to any one of claim 58 or 59, or a pharmaceutically acceptable salt thereof, wherein the C₁-C₃alkoxy is OCH₃.

61. The compound according to any one of claim 1 or 2, wherein the compound of Formula I is a compound of Formula I-K: 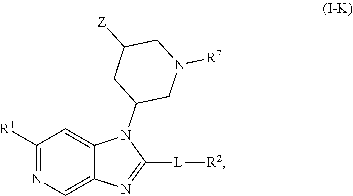
or a pharmaceutically acceptable salt thereof, wherein:
R⁷ is hydrogen, C₁-C₆alkyl, C(O)C₁-C₆alkyl, oxo, OH, C₁-C₆alkoxy, C(O)NR^{f}R^{g}, C₁-C₆hydroxyalkyl, CO₂(C₁-C₆alkyl), SO₂(C₁-C₆alkyl), or SO₂(C₃-C₆cycloalkyl); and
each R^{f} and R^{g} is independently hydrogen or C₁-C₄alkyl.

62. The compound according to claim 61, wherein the compound of Formula I-K is a compound of Formula I-K-1: 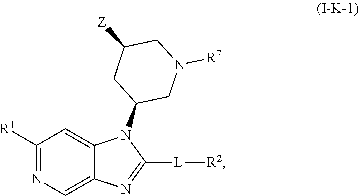
or a pharmaceutically acceptable salt thereof.

63. The compound according to any one of claim 1 or 11, wherein the compound of Formula II is a compound of Formula II-K: 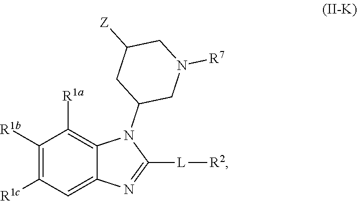
or a pharmaceutically acceptable salt thereof, wherein:
R⁷ is hydrogen, C₁-C₆alkyl, C(O)C₁-C₆alkyl, oxo, OH, C₁-C₆alkoxy, C(O)NR^{f}R^{g}, C₁-C₆hydroxyalkyl, CO₂(C₁-C₆alkyl), SO₂(C₁-C₆alkyl), or SO₂(C₃-C₆cycloalkyl); and
each R^{f} and R^{g} is independently hydrogen or C₁-C₄alkyl.

64. The compound according to claim 63, wherein the compound of Formula II-K is a compound of Formula II-K-1: 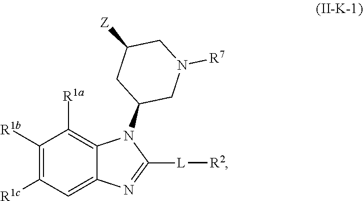
or a pharmaceutically acceptable salt thereof.

65. The compound of any one of claims 61-64, or a pharmaceutically acceptable salt thereof, wherein R⁷ is C₁-C₄alkyl or C(O)C₁-C₄alkyl.

66. The compound according to claim 65, or a pharmaceutically acceptable salt thereof, wherein R⁷ is CH₃, CH₂CH₃, or C(O)CH₃.

67. The compound according to claims 1, 2, 11, 20, 21, or 29-66 or a pharmaceutically acceptable salt thereof, wherein Z is —NHC(O)Ar¹.

68. The compound according to any one of claims 1-3, 11, 12, 20, 21, or 29-67, or a pharmaceutically acceptable salt thereof, wherein Ar¹ is optionally substituted 6-membered heteroaryl or optionally substituted phenyl.

69. The compound according to claim 68, or a pharmaceutically acceptable salt thereof, wherein the optionally substituted 6-membered heteroaryl is pyridinyl, pyrimidinyl, or pyridazinyl, each optionally substituted with one or more of halo, C₁-C₆alkyl, C₃-C₆cycloalkyl, C₁-C₆haloalkyl, C₁-C₆alkoxy, C₁-C₆haloalkoxy, C₁-C₆hydroxyalkyl, cyano, OH, oxo, or NR^{j}R^{k}; and each R^{j} and R^{k} is independently hydrogen or C₁-C₄alkyl.

70. The compound according to claim 69, or a pharmaceutically acceptable salt thereof, wherein Ar¹ is

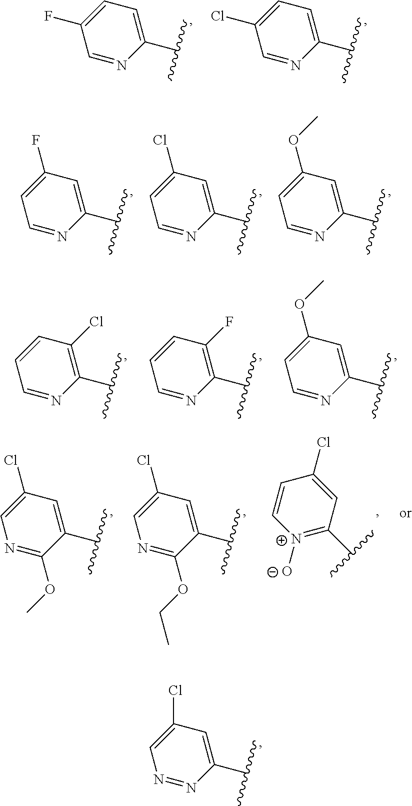

71. The compound according to claim 68, or a pharmaceutically acceptable salt thereof, wherein:
Ar¹ is 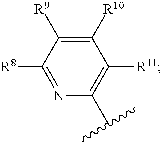
R⁸, R⁹, R¹⁰, and R¹¹ are each independently hydrogen, halo, C₁-C₄alkyl, C₁-C₄haloalkyl, C₁-C₄alkoxy, or C₁-C₄haloalkoxy.

72. The compound according to claim 71, or a pharmaceutically acceptable salt thereof, wherein at least two of R⁸, R⁹, R¹⁰, and R¹¹ are hydrogen.

73. The compound according to claim 68, or a pharmaceutically acceptable salt thereof, wherein:
Ar¹ is phenyl, optionally substituted with one or more of halo, C₁-C₆alkyl, C₃-C₆cycloalkyl, C₁-C₆haloalkyl, C₁-C₆alkoxy, C₁-C₆haloalkoxy, cyano, OH, NR^{j}R^{k}, C(O)NR^{f}R^{g}, CO₂(C₁-C₆alkyl), SO₂(C₁-C₄alkyl), SO₂(C₃-C₄cycloalkyl), C₁-C₄alkylene-R¹², or O—R¹³;
R¹² is cyano, NR^{j}R^{k}, OH, or C₁-C₄alkoxy;
R¹³ is C₁-C₆hydroxyalkyl, C₃-C₆cycloalkyl, optionally substituted 4-, 5-, or 6-membered heterocycloalkyl, (C₁-C₄alkylene)C₁-C₄alkoxy, (C₁-C₄alkylene)NR^{j}R^{k}, or (C₁-C₄alkylene)-(optionally substituted 4-, 5-, or 6-membered heterocycloalkyl), and
each R^{f}, R^{g}, R^{j}, and R^{k} is independently hydrogen or C₁-C₄alkyl.

74. The compound according to claim 73, or a pharmaceutically acceptable salt thereof, wherein Ar¹ is

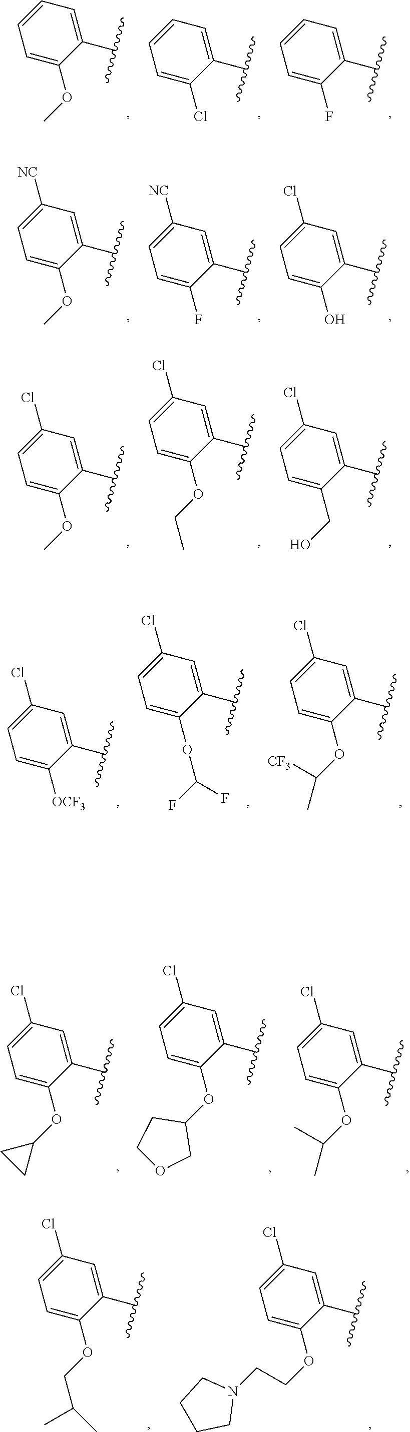

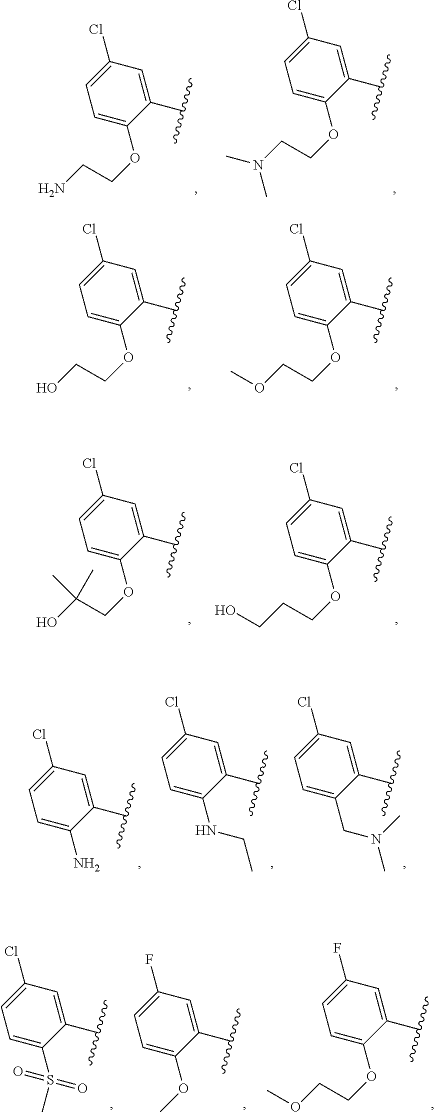

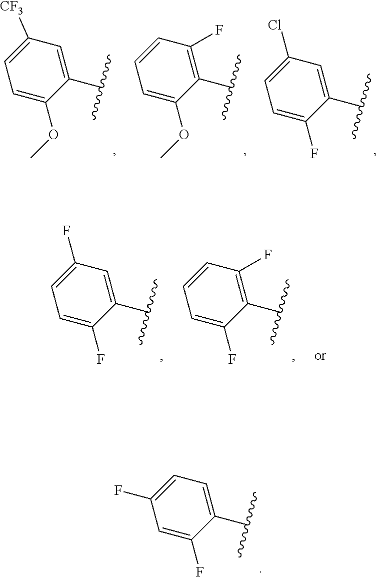

75. The compound according to claim 68, or a pharmaceutically acceptable salt thereof, wherein:
Ar¹ is 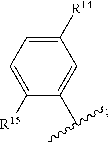
R¹⁴ is hydrogen, halo, cyano, or C₁-C₆haloalkyl;
R¹⁵ is halo, C₁-C₆alkoxy, C₁-C₆haloalkoxy, OH, NR^{j}R^{k}, C₁-C₄alkylene-R¹⁶, or O—R¹⁷;
R¹⁶ is NR^{j}R^{k} or OH;
R¹⁷ is C₁-C₆hydroxyalkyl, C₃-C₆cycloalkyl, optionally substituted 4-, 5-, or 6-membered heterocycloalkyl, (C₁-C₄alkylene)C₁-C₄alkoxy, (C₁-C₄alkylene)NR^{j}R^{k}, or (C₁-C₄alkylene)-(optionally substituted 4-, 5-, or 6-membered heterocycloalkyl); and
each R^{j} and R^{k} is independently hydrogen or C₁-C₄alkyl.

76. The compound according to any one of claims 1-3, 11, 12, 20, 21, or 29-67, or a pharmaceutically acceptable salt thereof, wherein Ar¹ is

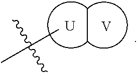

77. The compound according to claim 76, or a pharmaceutically acceptable salt thereof, wherein U is optionally substituted phenyl, and V is optionally substituted 5-membered heterocycloalkyl or optionally substituted 5-membered heteroaryl.

78. The compound according to claim 77, or a pharmaceutically acceptable salt thereof, wherein V is optionally substituted furanyl, optionally substituted isofuranyl, optionally substituted oxazolyl, optionally substituted isoxazolyl, optionally substituted thiazolyl, optionally substituted isothiazolyl, optionally substituted pyrrolyl, optionally substituted pyrazolyl, optionally substituted imidazolyl, or optionally substituted thiophenyl.

79. The compound according to claim 77 or 78, or a pharmaceutically acceptable salt thereof, wherein V is unsubstituted furanyl, unsubstituted isofuranyl, unsubstituted oxazolyl, unsubstituted isoxazolyl, unsubstituted thiazolyl, unsubstituted isothiazolyl, unsubstituted pyrrolyl, unsubstituted pyrazolyl, unsubstituted imidazolyl, or unsubstituted thiophenyl.

80. The compound according to any one of claims 76-79, or a pharmaceutically acceptable salt thereof, wherein U is phenyl substituted with one or more of halo, cyano, or C₁-C₆haloalkyl.

81. The compound according to claim 80, or a pharmaceutically acceptable salt thereof, wherein U is phenyl substituted with one or more halo; and V is unsubstituted furanyl.

82. The compound according to any one of claims 1, 2, 11, 20-23, or 29-67, or a pharmaceutically acceptable salt thereof, wherein:
Z is 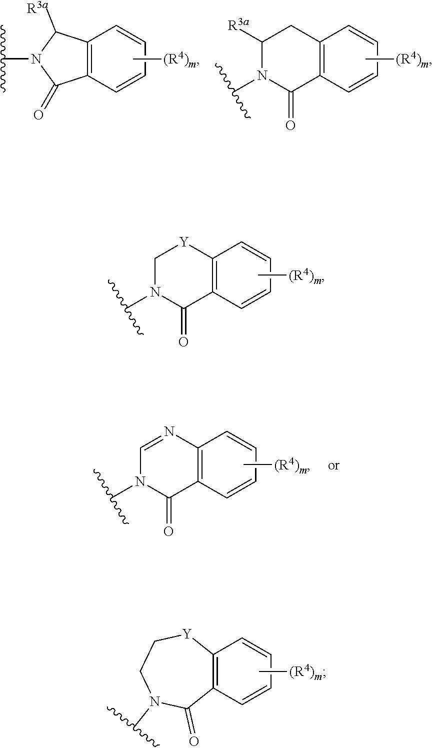
and
R^{3a} is hydrogen or oxo.

83. The compound according to claim 82, or a pharmaceutically acceptable salt thereof, wherein each R⁴ is independently halo and m is 1 or 2.

84. The compound according to claim 83, or a pharmaceutically acceptable salt thereof, wherein each R⁴ is independently F or Cl.

85. The compound according to claim 84, or a pharmaceutically acceptable salt thereof, wherein Z is

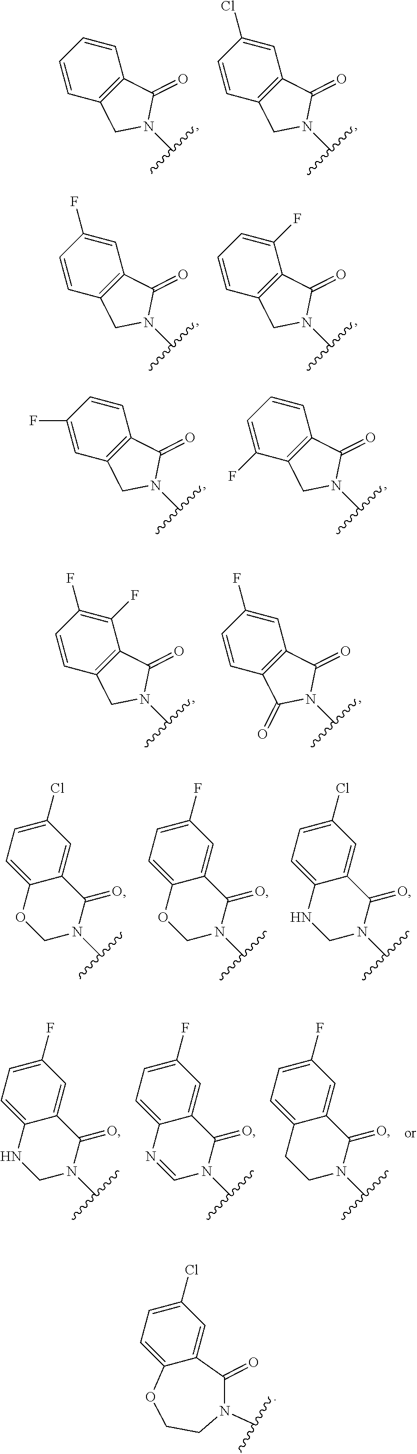

86. The compound according to any one of claims 1-10, 20-48, 51-55, 58-62, or 65-85, or a pharmaceutically acceptable salt thereof, wherein R¹ is optionally substituted heteroaryl.

87. The compound according to claim 86, or a pharmaceutically acceptable salt thereof, wherein R¹ is optionally substituted 5- or 6-membered heteroaryl.

88. The compound according to claim 87, or a pharmaceutically acceptable salt thereof, wherein R¹ is optionally substituted 5-membered heteroaryl.

89. The compound according to claim 88, or a pharmaceutically acceptable salt thereof, wherein the 5-membered heteroaryl is optionally substituted oxadiazolyl, optionally substituted thiadiazolyl, optionally substituted oxazolyl, optionally substituted thiazolyl, optionally substituted pyrazolyl, optionally substituted imidazolyl, optionally substituted triazolyl, or optionally substituted tetrazolyl.

90. The compound according to claim 88 or claim 89, or a pharmaceutically acceptable salt thereof, wherein the 5-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, CO₂(C₁-C₆alkyl), or C(O)NR^{f}R^{g}; and wherein each R^{f}, R^{g}, R^{h} and Rⁱ is independently hydrogen or C₁-C₄alkyl.

91. The compound according to claim 87, or a pharmaceutically acceptable salt thereof, wherein R¹ is optionally substituted 6-membered heteroaryl.

92. The compound according to claim 91, or a pharmaceutically acceptable salt thereof, wherein the optionally substituted 6-membered heteroaryl is optionally substituted pyridinyl, optionally substituted pyrimidinyl, or optionally substituted pyridazinyl.

93. The compound according to claim 91 or claim 92, or a pharmaceutically acceptable salt thereof, wherein the 6-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, CO₂(C₁-C₆alkyl), or C(O)NR^{f}R^{g}; and wherein each R^{f}, R^{g}, R^{h} and Rⁱ is independently hydrogen or C₁-C₄alkyl.

94. The compound according to claim 87, or a pharmaceutically acceptable salt thereof, wherein:
R¹ is 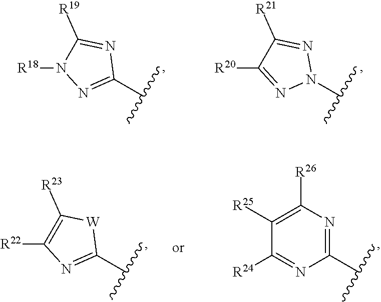
R¹⁸ is hydrogen, C₁-C₆alkyl, or C₁-C₆haloalkyl;
R¹⁹ is hydrogen or C₁-C₆alkyl;
R²⁰ and R²¹ are each independently hydrogen, C₁-C₆alkyl, C₁-C₆haloalkyl, C₁-C₆hydroxyalkyl, or C₀₂(C₁-C₆alkyl);
W is O or S;
R²² is hydrogen, C₁-C₆alkyl, C₁-C₆haloalkyl, halo, C₁-C₆hydroxyalkyl, or NR^{h}Rⁱ;
R²³ is hydrogen or C₁-C₆alkyl;
R²⁴, R²⁵, and R²⁶ are each independently hydrogen, halo, cyano, C₁-C₆alkyl, C₁-C₆haloalkyl, or C₃-C₆cycloalkyl; and
each R^{h} and Rⁱ is independently hydrogen or C₁-C₄alkyl.

95. The compound according to claim 86, or a pharmaceutically acceptable salt thereof, wherein R¹ is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.

96. The compound according to any one of claims 1-10, 20-48, 51-55, 58-62, or 65-85, or a pharmaceutically acceptable salt thereof, wherein R¹ is optionally substituted aryl.

97. The compound according to claim 96, or a pharmaceutically acceptable salt thereof, wherein the optionally substituted aryl is phenyl, optionally substituted with C₁-C₆alkyl, C₁-C₆haloalkyl, C₁-C₆hydroxyalkyl, halo, cyano, or C₁-C₆cyanoalkyl.

98. The compound according to any one of claims 1-10, 20-48, 51-55, 58-62, or 65-85, or a pharmaceutically acceptable salt thereof, wherein R¹ is optionally substituted 4-, 5- or 6-membered heterocycloalkyl, such as optionally substituted azetidinyl, optionally substituted pyrrolidinyl, optionally substituted tetrahydropyranyl, optionally substituted piperidinyl, optionally substituted morpholinyl, or optionally substituted dioxanyl.

99. The compound according to claim 98, or a pharmaceutically acceptable salt thereof, wherein R¹ is pyrrolidinyl optionally substituted with one or more of oxo, C₁-C₆hydroxyalkyl, C₁-C₆alkyl, C₁-C₆alkoxy(alkylene), C₁-C₆cyanoalkyl, OH, cyano, C(O)NR^{f}R^{g}, and wherein each R^{f} and R^{g} is independently hydrogen or C₁-C₄alkyl.

100. The compound according to any one of claims 1, 11-19, 22-46, 49-53, 56-60, or 63-85, or a pharmaceutically acceptable salt thereof, wherein R^{1b} is optionally substituted heteroaryl.

101. The compound according to claim 100, or a pharmaceutically acceptable salt thereof, wherein R^{1b} is optionally substituted 5- or 6-membered heteroaryl.

102. The compound according to claim 101, or a pharmaceutically acceptable salt thereof, wherein the optionally substituted 5- or 6-membered heteroaryl is optionally substituted triazolyl, optionally substituted oxadiazolyl, optionally substituted thiadiazolyl, optionally substituted oxazolyl, optionally substituted thiazolyl, optionally substituted pyrazolyl, optionally substituted imidazolyl, optionally substituted tetrazolyl, optionally substituted pyridinyl, optionally substituted pyrimidinyl, or optionally substituted pyridazinyl.

103. The compound according to any one of claim 101 or 102, or a pharmaceutically acceptable salt thereof, wherein the optionally substituted 5- or 6-membered heteroaryl is substituted with one or more of oxo, NR^{h}Rⁱ, C₁-C₆alkyl, C₁-C₆haloalkyl, C₁-C₆hydroxyalkyl, halo, cyano, C₁-C₆alkoxy, C₁-C₆haloalkoxy, CO₂(C₁-C₆alkyl), or C(O)NR^{f}R^{g}; and wherein each R^{f}, R^{g}, R^{h} and Rⁱ is independently hydrogen or C₁-C₄alkyl.

104. The compound according to claim 101, or a pharmaceutically acceptable salt thereof, wherein:
R^{1b} is 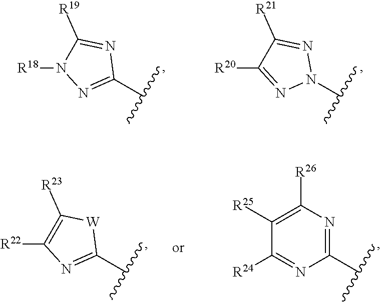
R¹⁸ is hydrogen, C₁-C₆alkyl, or C₁-C₆haloalkyl;
R¹⁹ is hydrogen or C₁-C₆alkyl;
R²⁰ and R²¹ are each independently hydrogen, C₁-C₆alkyl, C₁-C₆haloalkyl, C₁-C₆hydroxyalkyl, or C₀₂(C₁-C₆alkyl);
W is O or S;
R²² is hydrogen, C₁-C₆alkyl, C₁-C₆haloalkyl, halo, C₁-C₆hydroxyalkyl, or NR^{h}Rⁱ;
R²³ is hydrogen or C₁-C₆alkyl;
R²⁴, R²⁵, and R²⁶ are each independently hydrogen, halo, cyano, C₁-C₆alkyl, C₁-C₆haloalkyl, or C₃-C₆cycloalkyl; and
each R^{h} and Rⁱ is independently hydrogen or C₁-C₄alkyl.

105. The compound according to claim 100, or a pharmaceutically acceptable salt thereof, wherein R^{1b} is optionally substituted 9-membered heteroaryl, such as optionally substituted benzofuranyl, optionally substituted isobenzofuranyl, optionally substituted benzoxazolyl, optionally substituted benzoisoxazolyl, optionally substituted benzothiazolyl, optionally substituted benzoisothiazolyl, optionally substituted indolyl, optionally substituted isoindolyl, optionally substituted indazolyl, optionally substituted benzimidazolyl, or optionally substituted benzothiophenyl.

106. The compound according to any one of claims 1, 11-19, 22-46, 49-53, 56-60, or 63-85, or a pharmaceutically acceptable salt thereof, wherein R^{1b} is optionally substituted aryl.

107. The compound according to claim 106, or a pharmaceutically acceptable salt thereof, wherein the optionally substituted aryl is phenyl, optionally substituted with C₁-C₆alkyl, C₁-C₆haloalkyl, C₁-C₆hydroxyalkyl, halo, cyano, or C₁-C₆cyanoalkyl.

108. The compound according to any one of claims 1, 11-19, 22-46, 49-53, 56-60, or 63-85, or a pharmaceutically acceptable salt thereof, wherein R^{1b} is optionally substituted 4-, 5- or 6-membered heterocycloalkyl, such as optionally substituted azetidinyl, optionally substituted pyrrolidinyl, optionally substituted tetrahydropyranyl, optionally substituted piperidinyl, optionally substituted morpholinyl, or optionally substituted dioxanyl.

109. The compound according to claim 108, or a pharmaceutically acceptable salt thereof, wherein R^{1b} is pyrrolidinyl, optionally substituted with one or more of oxo, C₁-C₆hydroxyalkyl, C₁-C₆alkyl, C₁-C₆alkoxy(alkylene), C₁-C₆cyanoalkyl, OH, cyano, C(O)NR^{f}R^{g}, and wherein each R^{f} and R^{g} is independently hydrogen or C₁-C₄alkyl.

110. The compound according to any one of claims 1, 11-19, 22-46, 49-53, 56-60, 63-85, or 100-109, or a pharmaceutically acceptable salt thereof, wherein R^{1a} is hydrogen.

111. The compound according to any one of claims 1, 11-19, 22-46, 49-53, 56-60, 63-85, or 100-109, or a pharmaceutically acceptable salt thereof, wherein R^{1a} is fluoro.

112. The compound according to any one of claims 1, 11-19, 22-46, 49-53, 56-60, 63-85, or 100-111, or a pharmaceutically acceptable salt thereof, wherein R^{1c} is hydrogen.

113. The compound according to any one of claims 1-112, or a pharmaceutically acceptable salt thereof, wherein L is a bond.

114. The compound according to any one of claims 1-112, or a pharmaceutically acceptable salt thereof, wherein L is —O—.

115. The compound according to any one of claims 1-112, or a pharmaceutically acceptable salt thereof, wherein L is —NR^{a}—, wherein R^{a} is hydrogen or C₁-C₄alkyl.

116. The compound according to any one of claims 1-112, or a pharmaceutically acceptable salt thereof, wherein L is optionally substituted C₁-C₄alkylene.

117. The compound according to claim 116, or a pharmaceutically acceptable salt thereof, wherein L is C₁-C₄alkylene substituted with deuterium, oxo, halo, NH₂, NH(C₁-C₄alkyl), N(C₁-C₄alkyl)₂, OH, C₁-C₃alkoxy, NR¹C(O)(C₁-C₄alkyl), NR¹CO₂(C₁-C₄alkyl), or an optionally substituted 4-, 5-, or 6-membered heterocycloalkyl containing a nitrogen wherein the nitrogen is connected to the alkylene carbon, and each R¹ is independently hydrogen or C₁-C₄alkyl.

118. The compound according to claim 116, or a pharmaceutically acceptable salt thereof, wherein L is unsubstituted C₁-C₄alkylene.

119. The compound according to any one of claims 1-118, or a pharmaceutically acceptable salt thereof, wherein R² is optionally substituted phenyl, optionally substituted pyridinyl, optionally substituted C₁-C₆alkyl, optionally substituted C₁-C₆alkoxy, or NR^{b}R^{c}.

120. The compound according to any one of claims 1-119, or a pharmaceutically acceptable salt thereof, wherein:
R² is phenyl or pyridinyl, each optionally substituted with one or more of halo, C₁-C₆alkyl, C₃-C₆cycloalkyl, C₁-C₆alkoxy, C₁-C₆haloalkoxy, cyano, OH, NR^{m}Rⁿ, C(O)NR^{f}R^{g}, C₀₂(C₁-C₆alkyl), COOH, C₁-C₄alkylene-R²⁷, or O—R²⁸;
R²⁷ is cyano, NR^{m}Rⁿ, or COOH;
R²⁸ is C₁-C₆hydroxyalkyl, C₃-C₆cycloalkyl, optionally substituted 4-, 5-, or 6-membered heterocycloalkyl, or (C₁-C₄alkylene)C₁-C₄alkoxy; and
each R^{f}, R^{g}, R^{m}, and Rⁿ is independently hydrogen or C₁-C₄alkyl.

121. The compound according to any one of claims 1-113, or a pharmaceutically acceptable salt thereof, wherein:
L is a bond;
R³⁵ 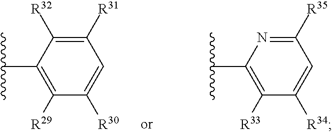
R²⁹, R³⁰, R³¹, R³², R³³, R³⁴, and R³⁵ are each independently hydrogen, halo, OH, cyano, C₁-C₄alkoxy, C₁-C₄haloalkoxy, C(O)NR^{f}R^{g}, or O—R²⁸;
R²⁸ is C₁-C₆hydroxyalkyl or (C₁-C₄alkylene)C₁-C₄alkoxy; and
wherein each R^{f} and R^{g} is independently hydrogen or C₁-C₄alkyl.

122. The compound according to claim 121, or a pharmaceutically acceptable salt thereof, wherein R² is 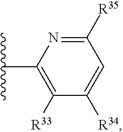
R³⁴ and R³⁵ are each hydrogen, and R³³ is hydrogen, halo, or C₁-C₄haloalkoxy.

123. The compound according to claim 122, or a pharmaceutically acceptable salt thereof, wherein R³³ is hydrogen, F, or OCHF₂.

124. The compound according to claim 121, or a pharmaceutically acceptable salt thereof, wherein R² is 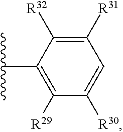
and at least two of R²⁹, R³⁰, R³¹, and R³² are hydrogen.

125. The compound according to claim 124, or a pharmaceutically acceptable salt thereof, wherein one of R²⁹ and R³² is halo, OH, C₁-C₄alkoxy, or C₁-C₄haloalkoxy and the other of R²⁹ and R³² is halo; and R³⁰ and R³¹ are hydrogen.

126. The compound according to claim 124, or a pharmaceutically acceptable salt thereof, wherein R²⁹ is halo, cyano, OH, C₁-C₄haloalkoxy, C₁-C₄alkoxy, C(O)NR^{f}R^{g}, or O—R²⁸, and R³⁰, R³¹, and R³² are each hydrogen.

127. The compound according to claim 120, or a pharmaceutically acceptable salt thereof, wherein R² is

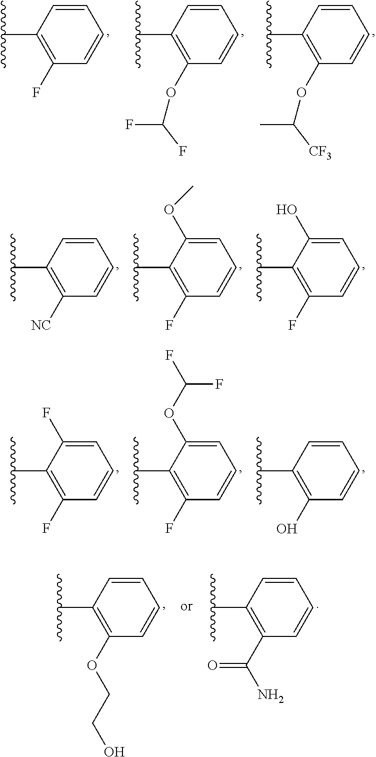

128. The compound according to any one of claims 1-119, or a pharmaceutically acceptable salt thereof, wherein R² is C₁-C₆alkyl, optionally substituted with cyano, OH, or C(O)NRⁱR^{j}, and wherein each Rⁱ and R^{j} is independently hydrogen or C₁-C₄alkyl.

129. The compound according to claim 128, or a pharmaceutically acceptable salt thereof, wherein R² is CH₃, CH₂CH₃, CH(CH₃)₂, CH₂CH(CH₃)₂, C(CH₃)₃, CH₂C(CH₃)₃, C(CH₃)₂OH, CH(OH)(CH₃), C(CH₃)₂CN, C(CH₃)₂C(O)NH₂, or C(CH₃)(CH₂CH₂OH).

130. The compound according to any one of claims 1-119, or a pharmaceutically acceptable salt thereof, wherein R² is C₁-C₆alkoxy, optionally substituted with OH.

131. The compound according to claim 130, or a pharmaceutically acceptable salt thereof, wherein R² is OCH₃, OCH(CH₃)₂, or OCH₂CH₂OH.

132. The compound according to any one of claims 1-118, or a pharmaceutically acceptable salt thereof, wherein R² is C₁-C₆haloalkyl or C₁-C₆haloalkoxy.

133. The compound according to claim 132, or a pharmaceutically acceptable salt thereof, wherein R² is CF₃, CH(CH₃)(CF₃), CH₂CF₃, C(CH₃)₂F, CH₂C(CH₃)₂F, C(CH₃)F₂, CH₂C(CH₃)F₂, CHFCH(CH₃)₂, CF₂CH(CH₃)₂, OCF₃, OCH₂CF₃, OCHF₂, OCH₂F, or OCH(CH₃)(CF₃).

134. The compound according to any one of claims 1-118, or a pharmaceutically acceptable salt thereof, wherein R² is C₃-C₆cycloalkyl, optionally substituted with one or more of C₁-C₄alkyl or C₁-C₄haloalkyl.

135. The compound according to any one of claims 1-119, or a pharmaceutically acceptable salt thereof, wherein R² is NR^{b}R^{c} and R^{b} and R^{c} are each independently hydrogen or C₁-C₄alkyl.

136. The compound according to claim 135, or a pharmaceutically acceptable salt thereof, wherein R² is NH(CH₃), N(CH₃)₂, N(CH₃)(CH₂CH₃), or NH(CH₂CH₃).

137. The compound according to any one of claims 1-118, or a pharmaceutically acceptable salt thereof, wherein R² is optionally substituted 5-membered heteroaryl, such as optionally substituted triazolyl, optionally substituted pyrazolyl, optionally substituted oxazolyl, or optionally substituted isoxazolyl.

138. The compound according to any one of claims 1-118, or a pharmaceutically acceptable salt thereof, wherein R² is optionally substituted 4-, 5- or 6-membered heterocycloalkyl, such as optionally substituted azetidinyl, optionally substituted tetrahydrofuranyl, optionally substituted pyrrolidinyl, optionally substituted tetrahydropyranyl, optionally substituted piperidinyl, optionally substituted morpholinyl, or optionally substituted dioxanyl.

139. The compound of claim 1 that is a compound of Tables 1, 2, 3, or 4, or a pharmaceutically acceptable salt thereof.

140. A pharmaceutical composition comprising a compound according to any one of claims 1-139, or a pharmaceutically acceptable salt thereof, and a pharmaceutically acceptable excipient.

141. A method of reducing plasma methylmalonic acid in a subject having an elevated methylmalonic acid level comprising administering to the subject a therapeutically effective amount of a compound of any one of claims 1-139, or a pharmaceutically acceptable salt thereof.

142. A method of treating a BCAA-catabolism-related diseases in a subject in need thereof comprising administering to the subject a therapeutically effective amount of a compound of any one of claims 1-139, or a pharmaceutically acceptable salt thereof.

143. The method according to claim 142, wherein the BCAA-catabolism-related disease is methylmalonic acidemia (MMA), propionic acidemia (PA), isovaleric acidemia (IVA), maple syrup urine disease, methylmalonic semialdehyde (MMSDH) deficiency, 3-hydroxyisobutyrate dehydrogenase (HIBADH) deficiency, 3-hydroxyisobutyryl-CoA hydrolase (HIBCH) deficiency, isobutyryl-CoA (IBD) deficiency, acetyl-CoA carboxylase 1 (ACC) deficiency, mitochondrial short-chain enoyl-CoA hydratase 1 (ECHS1) deficiency, methylbutyryl CoA dehydrogenase (SBCAD) deficiency, beta-ketothiolase deficiency (BKT), hydroxy-3-methylglutaconyl-CoA lysase (HMGCL) deficiency, 3-methylglutaconyl-CoA-hydratase deficiency, or 3-methylcrotonyl-CoA (3-MCC) deficiency.

144. A method of reducing blood ammonia in a subject having an elevated ammonia level comprising administering to the subject a therapeutically effective amount of a compound of any one of claims 1-139, or a pharmaceutically acceptable salt thereof.

145. A method of reducing blood 2-methylcitrate in a subject having an elevated 2-methylcitrate level comprising administering to the subject a therapeutically effective amount of a compound of any one of claims 1-139, or a pharmaceutically acceptable salt thereof.

146. A mouse (Mus musculus) as an animal model for Branched Chain Amino Acid Transaminase 2 (BCAT2) deficiency, wherein the mouse comprises one or more genomic mutations in an endogenous BCAT2 gene that results in a knockout of the endogenous BCAT2 gene.

147. The mouse of claim 146, wherein the one or more mutations are in exon 3 of the BCAT2 gene.

148. The mouse of claim 146 or 147, wherein the one or more mutations in exon 3 of the BCAT2 gene comprise a deletion of adenine (A) at position 245 of SEQ ID NO: 1.

149. The mouse of any one of claims 146 to 148, wherein the one or more mutations result in a premature stop codon in the BCAT2 gene.

150. The mouse of claim 149, wherein the premature stop codon is in exon 4 of the BCAT2 gene.

151. The mouse of claim 149 or 150, wherein the premature stop codon is a TGA stop codon at positions 344-346 of SEQ ID NO: 1.

152. The mouse of any one of claims 146 to 151, wherein the mouse is homozygous for the knockout of the endogenous BCAT2 gene.

153. The mouse of any one of claims 146 to 152, which is generated by CRISPR/Cas9-mediated homology-directed repair (HDR) that introduces the one or more genomic mutations into the endogenous BCAT2 gene.

154. The mouse of claim 153, wherein a single guide RNA (sgRNA) encoded by the polynucleotide sequence of SEQ ID NO: 8 and an sgRNA encoded by the polynucleotide sequence of SEQ ID NO: 9 are used for the CRISPR/Cas9-mediated HDR.

155. The mouse of any one of claims 146 to 154, wherein the mouse has increased plasma isoleucine levels relative to a mouse that does not comprise a knockout of the endogenous BCAT2 gene.

156. A mouse (Mus musculus) as an animal model for methylmalonic acidemia, wherein the mouse comprises one or more genomic mutations in at least one endogenous methylmalonyl Coenzyme A (MMA-CoA) mutase gene that results in an M698K substitution relative to the amino acid sequence of SEQ ID NO: 4 in a MMA-CoA protein encoded by the gene.

157. The mouse of claim 156, wherein the one or more genomic mutations is a T2228A substitution relative to the nucleic acid sequence of SEQ ID NO: 3.

158. The mouse of claim 156 or 157, which is homozygous for the one or more genomic mutations in the endogenous MMA-CoA mutase gene that results in the M698K substitution.

159. The mouse of claim 156 or 157, which is heterozygous for the one or more genomic mutations in the endogenous MMA-CoA mutase gene that results in the M698K substitution.

160. The mouse of claim 159, wherein the mouse comprises the one or more genomic mutations that results in the M698K substitution at a first locus of an endogenous MMA-CoA mutase gene, and further comprises one or more genomic mutations that result in a knockout of an endogenous MMA-CoA mutase gene at a second locus of an endogenous MMA-CoA mutase gene.

161. The mouse of claim 160, wherein the one or more genomic mutations that result in a knock out of the endogenous MMA-CoA mutase gene is in exon 2 of the endogenous MMA-CoA mutase gene.

162. The mouse of claim 160 or 161, wherein the one or more genomic mutations that result in a knock out of the endogenous MMA-CoA mutase gene introduce a premature stop codon into the endogenous MMA-CoA mutase gene.

163. The mouse of any one of claims 160 to 162, wherein the one or more genomic mutations that result in a knock out of the endogenous MMA-CoA mutase gene comprise a G311A substitution relative to the nucleic acid sequence of SEQ ID NO: 3.

164. The mouse of any one of claims 160 to 163, wherein the mouse is homozygous for the knockout of the endogenous MMA-CoA mutase gene.

165. The mouse of any one of claims 156 to 164, which is generated by CRISPR/Cas9-mediated homology-directed repair (HDR) that introduces the one or more genomic mutations into the endogenous MMA-CoA mutase gene that results in the M698K substitution.

166. The mouse of claim 165, wherein a single guide RNA (sgRNA) encoded by the polynucleotide sequence of SEQ ID NO: 19 and an sgRNA encoded by the polynucleotide sequence of SEQ ID NO: 20 are used for the CRISPR/Cas9-mediated HDR.

167. The mouse of claim 165 or 166, wherein the CRISPR/Cas9-mediated HDR utilizes a donor DNA having the polynucleotide sequence of SEQ ID NO: 21.

168. The mouse of any one of claims 160 to 167, which is generated by CRISPR/Cas9-mediated homology-directed repair (HDR) that introduces the one or more genomic mutations into the endogenous MMA-CoA mutase gene that results in a knockout of an endogenous MMA-CoA mutase gene.

169. The mouse of claim 168, wherein a single guide RNA (sgRNA) encoded by the polynucleotide sequence of SEQ ID NO: 14 is used for the CRISPR/Cas9-mediated HDR.

170. The mouse of any one of claims 156 to 169, wherein the mouse further comprises one or more genomic mutations in an endogenous BCAT2 gene that results in a knockout of the endogenous BCAT2 gene.

171. The mouse of claim 170, wherein the mouse is homozygous for the knockout of the endogenous BCAT2 gene.

172. The mouse of any one of claims 156 to 171, wherein the mouse has increased plasma levels of a compound selected from the group consisting of 3-OH-isobutyric acid (3-HIB), methylmalonic acid (MMA) and plasma keto isoleucine (Keto-Ile) relative to a syngeneic wildtype mouse.

173. A mouse (Mus musculus) as an animal model for propionic acidemia, wherein the mouse comprises one or more genomic mutations in at least one endogenous propionyl-Coenzyme A carboxylase α subunit (PCCA) gene that results in an A134T substitution relative to the amino acid sequence of SEQ ID NO: 6 in a PCCA protein encoded by the gene.

174. The mouse of claim 173, wherein the one or more genomic mutations is a G439A substitution relative to the nucleic acid sequence of SEQ ID NO: 5.

175. The mouse of claim 173 or 174, which is homozygous for the one or more genomic mutations in the endogenous PCCA gene that results in the A134T substitution.

176. The mouse of claim 173 or 174, which is heterozygous for the one or more genomic mutations in the endogenous PCCA gene that results in the A134T substitution.

177. The mouse of claim 173 or 174, wherein the mouse comprises the one or more genomic mutations that results in the A134T substitution at a first locus of an endogenous PCCA gene, and further comprises one or more genomic mutations that result in a knockout of an endogenous PCCA gene at a second locus of an endogenous PCCA gene.

178. The mouse of claim 177, wherein the one or more genomic mutations that result in a knock out of the endogenous PCCA gene are in exon 5 of the endogenous PCCA gene.

179. The mouse of claim 177 or 178, wherein the one or more genomic mutations that result in a knock out of the endogenous PCCA gene introduce a premature stop codon into the endogenous PCCA mutase gene.

180. The mouse of any one of claims 177 to 179, wherein the one or more genomic mutations that result in a knock out of the endogenous PCCA gene comprise the addition of one or more nucleotides to exon 5 of the endogenous PCCA gene.

181. The mouse of any one of claims 177 to 180, wherein the one or more genomic mutations that result in a knock out of the endogenous PCCA gene comprise the addition of a nucleotide at position 379 of SEQ ID NO: 5.

182. The mouse of claim 181, wherein the nucleotide is adenine.

183. The mouse of any one of claims 173 to 182, which is generated by CRISPR/Cas9-mediated homology-directed repair (HDR) that introduces the one or more genomic mutations into the endogenous PCCA gene that results in an A134T substitution.

184. The mouse of claim 183, wherein a single guide RNA (sgRNA) encoded by the polynucleotide sequence of SEQ ID NO: 31 is used for the CRISPR/Cas9-mediated HDR.

185. The mouse of claim 183 or 184, wherein the CRISPR/Cas9-mediated HDR utilizes a donor DNA having the polynucleotide sequence of SEQ ID NO: 32.

186. The mouse of any one of claims 173 to 185, which is generated by CRISPR/Cas9-mediated homology-directed repair (HDR) that introduces the one or more genomic mutations into the endogenous PCCA gene that results in a knockout of an endogenous PCCA gene.

187. The mouse of claim 186, wherein a single guide RNA (sgRNA) encoded by the polynucleotide sequence of SEQ ID NO: 26 is used for the CRISPR/Cas9-mediated HDR.

188. The mouse of any one of claims 173 to 187, wherein the mouse has increased plasma propionyl carnitine levels relative to a mouse that does not comprise the A134T substitution and/or a knockout in an endogenous PCCA gene.

189. The mouse of any one of claims 146 to 188, which is a male.

190. The mouse of any one of claims 146 to 188, which is a female.

191. The mouse of any one of claims 146 to 190, which is a C57BL/6 mouse.


SEQUENCE LISTING (36 sequences)

SEQ ID NO: 1 - DNA, 1596 bp, Mus musculus
  misc_feature 1..1596: note = Mouse BCAT2 gene
  source 1..1596: mol_type = genomic DNA
acagtcaccagccgcacacaccggatcatggctgcagccacactaggacaggtctgggcccgaaagcttctgcctgttccctggcttctatgtgggtccaaaagatgtgtgtcctccatattcaaggctgcagacctccagattcagatgaccaaagaaccacagaagaagccagcccctagccaggcactgctgtttgggaagaccttcacagaccacatgctgatggtggagtggaataacaaggctggatggggcccaccaaggatccagcccttccagaacctcacgctacacccagcctgctctggtctgcactactctctgcagctctttgagggcttgaaagcatacaaaggtggagaccagcaggtacgcctcttccggccctggctcaacatggacaggatgctacgttctgctaggcgcctctgcctgccagactttgacaagcaggagctgttggagtgtatccgccagctcattgaagtagacaaagactgggttcccgatggcaacggaacgagcctctacgtgcggcccgtgctgatcgggaatgagccctcgctgggtgttggtatggtcacacaagccctcctgtacgtcattctgtgccctgtgggctcctacttccctggagactccatgacacctgtctccctcctggcggacccttcattcgtcagagcctggataggcggagtcggtgactgcaagttggggggtgtgaccaggctgccctccaggaactatggacccactgtggctgtgcagcgagaagcccagaagagaggctgcgagcaggtcctctggctgtacgggccagaccaccagctcactgaggtgggcaccatgaacatctttgtctactggacccatgaagacggagtactggagctggtaacccccccactgaatggcgtcatcttgcctggagtagttcgacaaagtctgctggatctggccaggacttggggtgagttccgggtggcagaacgcaaggtcactatgaaggaattgaagcgggcgctggaggaaggccgagtgcgagaagtcttcggctcaggcactgcttgtcaggtctgcccggtgcaccaaatcctgtatgaaggcaagcaactccacatacctaccatggagaacggcccagagctcatcctgcgcttccagaaggaactgaaggctattcagtacggagccagtgcccacgattggatgtttcgggtgtgacggtgggggctgtgccctccctggttggatctgcagacgtgccgcatctagtccagcgtcctcgcttcccagagactcaagagaagtgcaatagggaaaggtctgtccagatgctggatctctggcaccccctcgtggacgttacactccaaagccttaagggcgggacccatgcaatttggacgccacctcaaattttgggtctcaggactcagctgctccgcgtcctggtgttcaagggagaaccttggcttcttctctatctccgttctcaggccggatccccggtgctcttcgtgtccttcccccccccccccttttctccgtacttgctgcaattttgaaataaaatgccaaagaacaaga

SEQ ID NO: 2 - protein, 400 aa, Mus musculus
  SITE 1..400: note = Mouse BCAT2 protein
MAAATLGQVWARKLLPVPWLLCGSKRCVSSIFKAADLQIQMTKEPQKKPAPSQALLFGKTFTDHMLMVEWNNKAGWGPPRIQPFQNLTLHPACSGLHYSLQLFEGLKAYKGGDQQVRLFRPWLNMDRMLRSARRLCLPDFDKQELLECIRQLIEVDKDWVPDGNGTSLYVRPVLIGNEPSLGVGMVTQALLYVILCPVGSYFPGDSMTPVSLLADPSFVRAWIGGVGDCKLGGVTRLPSRNYGPTVAVQREAQKRGCEQVLWLYGPDHQLTEVGTMNIFVYWTHEDGVLELVTPPLNGVILPGVVRQSLLDLARTWGEFRVAERKVTMKELKRALEEGRVREVFGSGTACQVCPVHQILYEGKQLHIPTMENGPELILRFQKELKAIQYGASAHDWMFRV

SEQ ID NO: 3 - DNA, 3675 bp, Mus musculus
  misc_feature 1..3675: note = Mouse methylmalonyl-Coenzyme A (MMA-CoA) mutase
  source 1..3675: mol_type = genomic DNA
gattggctgccgagctcctgcggcgagcgggtgtgaccccggcgtctgggttcggttctgaagtccgggcttggtccgggtgatccccaaacactgaccgttctcatttccttttgggagtattcatgcttcaccatgttgagagctaagaatcaactttttttgctatcgccccattacctgaagcagctaaacattccatcagcttccagatggaaacgccttctacaccagcagcaaccccttcacccagaatgggctgtactggccaaaaagcagctgaaaggcaaaaacccagaggaccttatatggcacaccccagaagggatctctataaagcccttatattccagggcagatactctggacttacccgaagaacttccaggagtgaagccattcacacggggaccatatcctaccatgtatacctataggccctggaccatccgtcagtatgcaggctttagtactgtggaagaaagcaataaattctataaggacaatattaaggctggtcagcaggggttgtctgttgcctttgacttggcaacacatcgtggttatgattcagacaaccccagagttcgtggagatgttggaatggctggagttgctattgacactgtagaagacaccaaaatcctatttgatggcattcctttagaaaaaatgtccgtttccatgactatgaacggagctgtcatcccagtcctggcaacatttatagtaactggggaagagcaaggtgtgccgaaggagaagctcactggcacaattcagaatgacatcctaaaggagtttatggtcagaaatacttatatttttcccccagagccatcgatgaaaattattgctgacattttccaatacacagcacagcacatgccaaaatttaattccatttcgattagcgggtaccatatgcaggaagcaggagctgatgccattttagaactggcctataccatcgcagatgggttggagtactgcagaactggactccaggctggactcacaattgatgaatttgcaccaaggttgtctttcttctggggaattggaatgaacttctacatggaaatagccaagatgcgagctgggagaagactgtgggctcacttaatagaaaaaatgttccagcctaaaaactctaaatctcttcttctaagagcacactgccagacatcggggtggtcacttactgagcaggatccttacaataacattgtccgcactgcaatcgaagccatggcagctgtgtttggaggaacccagtctttgcatacgaactcttttgatgaagcgctgggtttgcccactgtgaaaagtgcccggattgctcggaacacacagatcatcattcaggaagaatctgggatccccaaagtggcggatccttggggagggtcgtacatgatggagtcgctcacaaatgacgtttatgaggctgctctgaagttgatatatgaagttgaagaaatgggtggaatggccaaagctgtagctgaaggaatccctaaacttcgcattgaagaatgtgctgcccgaagacaagctagaatagattctggttctgaggtaattgttggagtaaataagtatcagttggaaaaagaagactctgtggaggtcctggccattgacaacacttcagtgcgtaagaagcagattgaaaaactcaagaagattaaatccagcagggatcaagctttggctgagcagtgtctcagtgcacttacccagtgtgctgccagtggagatggcaatattctggctctggcagtggatgcagctcgtgcaagatgtacagttggagaaatcacggatgccttgaaaaaagtatttggtgagcataaagctaatgatcgtatggtgagtggagcatatcggcaggagtttggagaaagtaaagagatcacatctgccatcaagagagttaataaattcatggaacgtgaaggtcgcagacctcgtcttcttgtggcaaaaatgggacaagatggccatgacaggggagccaaggtcattgctacaggatttgctgatcttggttttgatgtggacataggccctctttttcagactccccgtgaagtggcgcagcaagctgtggatgcagatgtccatgctgtgggtgtcagcacacttgctgctggtcataaaaccctcgttcctgagcttatcaaagaactcaccgccctggggcggccagatatccttgtcatgtgtgggggcgtgattccaccacaggattatgaatttctgtatgaagttggtgtttccaacgtctttggtcctggaacccggattccaagagctgctgtccaagtgcttgatgatattgagaagtgtttggcagagaagcagcagtctgtgtaacatctggatctttagttatgactgaagcagcagtctgtgtaacatctggatctttagttatgaccgaagaaggtagtggtaaatccgtttcgtcaggtaatcccagcagctcttagaatgcttgctttggaaacctcacattaaaagattctaactataaatgtgcagtcatgccaatacacatatttaaagaatctgagttcaaaaaaaaaaaaaaaacctctgaaaatcatttataccaaatatgttttaagtatattattctagaaaattaaaatggtatactatttagaaatagcttacaattcttataaataattgaacttttgttttgttttatcctttgacatggggtcttactatgtagcactggttgtcttgcaactaactctgtagaacagtctggcctcaaactcacagagatccacctgcctctgccttcctagtacagggattaaaggtatgtgcctccatgtcctgcaaattattaaacttaaaaattaaataaaattttaaaatttaaccgattcttccttcaatttagtgttgatttgtgtactagaaactcaagttcctgtgttaatttaataaaataatgacatttatcttacaagaaatagtggagggtatttatggatggatcctcagccagacctactattgcttcaataaaagtagtgtttatcaccccctgtacaatggaataggggacatggtagataggccttccaggattgtgtcagctcctcacatgggaatgatattgttaatgtcaggttcaaaactctggaaaagatgtgtgtgacgctctcttaaatgaatgttgatggacagatgcatttgctataatgacagtctaaatctattacataaagtaaatgaattttcagatctagaaacacgctgatctgtatacactttcttattaatataagttataaaaataaaaaaatagtctgaagaactcttgtagtgtgatgttacttcactcattgaaggaagagactcactctgtgttaacctaatgtattcagcgaaggtgaggtgagcaacaaggattcttttgtaactttctacattatagatcaaataatattggggagggaaaatcatataatgctgtaaaattaataaactatactatagtttaatttttagaccatgactcttaggagggcatcatgattattgttttctattttaaagatgagcagtttgacgtcaaggacaacaatatatttattgtgttatcttatgtactacttataaagaaatatatatttgataaatacg

SEQ ID NO: 4 - protein, 748 aa, Mus musculus
  SITE 1..748: note = Mouse methylmalonyl-Coenzyme A mutase
MLRAKNQLFLLSPHYLKQLNIPSASRWKRLLHQQQPLHPEWAVLAKKQLKGKNPEDLIWHTPEGISIKPLYSRADTLDLPEELPGVKPFTRGPYPTMYTYRPWTIRQYAGFSTVEESNKFYKDNIKAGQQGLSVAFDLATHRGYDSDNPRVRGDVGMAGVAIDTVEDTKILFDGIPLEKMSVSMTMNGAVIPVLATFIVTGEEQGVPKEKLTGTIQNDILKEFMVRNTYIFPPEPSMKIIADIFQYTAQHMPKFNSISISGYHMQEAGADAILELAYTIADGLEYCRTGLQAGLTIDEFAPRLSFFWGIGMNFYMEIAKMRAGRRLWAHLIEKMFQPKNSKSLLLRAHCQTSGWSLTEQDPYNNIVRTAIEAMAAVFGGTQSLHTNSFDEALGLPTVKSARIARNTQIIIQEESGIPKVADPWGGSYMMESLTNDVYEAALKLIYEVEEMGGMAKAVAEGIPKLRIEECAARRQARIDSGSEVIVGVNKYQLEKEDSVEVLAIDNTSVRKKQIEKLKKIKSSRDQALAEQCLSALTQCAASGDGNILALAVDAARARCTVGEITDALKKVFGEHKANDRMVSGAYRQEFGESKEITSAIKRVNKFMEREGRRPRLLVAKMGQDGHDRGAKVIATGFADLGFDVDIGPLFQTPREVAQQAVDADVHAVGVSTLAAGHKTLVPELIKELTALGRPDILVMCGGVIPPQDYEFLYEVGVSNVFGPGTRIPRAAVQVLDDIEKCLAEKQQSV

SEQ ID NO: 5 - DNA, 2603 bp, Mus musculus
  misc_feature 1..2603: note = Mouse propionyl-Coenzyme A carboxylase gene
  source 1..2603: mol_type = genomic DNA
ggtgagaggtcagcagccggcaagtctgcggggtggacgatggcggggcagtgggtcaggaccgtggcgctgttggcggccaggcggcattggcggcggtcctcgcagcagcaattgctggggacgctgaagcatgctccagtctattcataccaatgcctagtggtgtccagaagtctcagttctgtggaatatgagcctaaagaaaagacttttgataaaattctcattgctaacagaggagaaattgcctgtagggttattaaaacttgcaagaagatgggcatcaagacagttgccattcacagtgatgttgatgccagttctgttcacgtgaaaatggcggatgaggctgtctgtgttggcccagctcccaccagtaaaagctacctcaacatggatgccatcatggaagccattaagaaaaccagggcccaagctgtacacccagggtatggattcctgtcagaaaacaaagagtttgcaaagcgtctggcagcagaagatgtcactttcattggacctgatactcatgctattcaagccatgggtgacaagatagaaagcaaactattagccaagagagcaaaggtcaacacaatccctggttttgatggggtagtaaaggatgcagatgaagctgtcagaattgcaagggaaattggctaccctgtgatgatcaaggcctcagcaggcggtggtgggaaaggcatgcgcatcgcctgggatgacgaagagaccagggatggctttagattttcatcccaggaagctgcttctagttttggtgatgatagactactaatagaaaaatttatcgataaccctcgtcatatagaaatccaggttttaggggataaacatggcaatgctctgtggctcaatgagagggagtgctcgatccagagaaggaaccagaaggtggtagaggaggcgccaagcatttttctggatcctgaaactcgccaagcaatgggagagcaggccgtggctttggctaaagccgtgaagtattcttctgctggaactgtggaatttcttgtggactcccagaagaatttttacttcttggagatgaatacaagactacaggtcgaacatcctgtcacagagtgcattactggcctggacttagtccaagaaatgatccttgttgctaagggttacccactcaggcacaagcaagaggatattcccatcagtggctgggcagttgaatgtcgggtttatgctgaggacccctacaagtctttcggtttaccgtctattgggaggctgtcccagtaccaagagccgatacatctacctggtgtccgagttgacagtggcatccaaccaggaagtgacatcagcatctattatgatcctatgatttcaaagctagtcacatatgggtctgacagagcagaagccctgaagaggatggaagacgcactggacaattatgtgatccggggtgttacacacaacatcccattgctccgggaggtgataatcaacacacgttttgtgaaaggagacatcagcactaagtttctctctgatgtgtatcctgatggcttcaaagggcacacgttaacactgagtgagagaaaccagttattggccattgcatcatctgtatttgtggcatcccagctacgagctcagcgcttccaagaacattcaagagtaccagttattaggcctgatgtggctaagtgggagctctcggtaaagttacatgatgaagatcatactgtcgtggcatctaacaatgggccggcatttaccgtggaagttgatggctcgaaactaaatgtgaccagtacgtggaacctggcgtcacccttattgtctgtcaacgttgatggcacgcagaggactgtgcagtgtctttctcgggaagcaggtggaaacatgagcatccagtttcttggcacagtgtacaaagtgcacattttaaccaagcttgctgcagagctgaacaaattcatgcttgaaaaagtgcccaaggacaccagcagcactctgtgctccccgatgcctggagtggtggtggccgtttctgtcaagcctggagacatggtagcagaaggtcaggaaatctgtgtgattgaagctatgaaaatgcagaacagtatgacagctgggaaaatgggcaaggtgaaattggtgcactgcaaagctggagacacagttggtgaaggagacctgcttgtggagctggaatgaagtatttctagcctttccgtcagcactgctcactgctttaactggcgtctccattattctctaaccctcaatttgcgcaagcatttacaggagcactcctgtgcaggaggttctacagcctcctgtttattcctcagaagaggcaaaaccgacattctgccaacaaatcaccaatggaaattcttgatataaatacttgtacatatgatttgtacttctgctgagatttcctagtgtcaaaattaaatcaataaaaccaagcatttgtctaaatattagtttgcccttttttccgaatgaagacaatatacacagaggcgacaggctgtgccaattgactgtaaatattagcatttcattgtgattctttaaaaatttatataaattcc

SEQ ID NO: 6 - protein, 724 aa, Mus musculus
  SITE 1..724: note = Mouse propionyl-Coenzyme A carboxylase protein
MAGQWVRTVALLAARRHWRRSSQQQLLGTLKHAPVYSYQCLVVSRSLSSVEYEPKEKTFDKILIANRGEIACRVIKTCKKMGIKTVAIHSDVDASSVHVKMADEAVCVGPAPTSKSYLNMDAIMEAIKKTRAQAVHPGYGFLSENKEFAKRLAAEDVTFIGPDTHAIQAMGDKIESKLLAKRAKVNTIPGFDGVVKDADEAVRIAREIGYPVMIKASAGGGGKGMRIAWDDEETRDGFRFSSQEAASSFGDDRLLIEKFIDNPRHIEIQVLGDKHGNALWLNERECSIQRRNQKVVEEAPSIFLDPETRQAMGEQAVALAKAVKYSSAGTVEFLVDSQKNFYFLEMNTRLQVEHPVTECITGLDLVQEMILVAKGYPLRHKQEDIPISGWAVECRVYAEDPYKSFGLPSIGRLSQYQEPIHLPGVRVDSGIQPGSDISIYYDPMISKLVTYGSDRAEALKRMEDALDNYVIRGVTHNIPLLREVIINTRFVKGDISTKFLSDVYPDGFKGHTLTLSERNQLLAIASSVFVASQLRAQRFQEHSRVPVIRPDVAKWELSVKLHDEDHTVVASNNGPAFTVEVDGSKLNVTSTWNLASPLLSVNVDGTQRTVQCLSREAGGNMSIQFLGTVYKVHILTKLAAELNKFMLEKVPKDTSSTLCSPMPGVVVAVSVKPGDMVAEGQEICVIEAMKMQNSMTAGKMGKVKLVHCKAGDTVGEGDLLVELE

SEQ ID NO: 7 - DNA, 22 bp, synthetic construct
  misc_feature 1..22: note = Synthetic
  source 1..22: mol_type = other DNA
aaaagcaccgactcggtgccac

SEQ ID NO: 8 - DNA, 20 bp, synthetic construct
  misc_feature 1..20: note = Synthetic
  source 1..20: mol_type = other DNA
gtggagtggaataacaaggc

SEQ ID NO: 9 - DNA, 20 bp, synthetic construct
  misc_feature 1..20: note = Synthetic
  source 1..20: mol_type = other DNA
aggctggatggggcccacca

SEQ ID NO: 10 - DNA, 20 bp, synthetic construct
  misc_feature 1..20: note = Synthetic
  source 1..20: mol_type = other DNA
gggtctaagggaaaagcccg

SEQ ID NO: 11 - DNA, 20 bp, synthetic construct
  misc_feature 1..20: note = Synthetic
  source 1..20: mol_type = other DNA
tatcggtgtaagatgccccc

SEQ ID NO: 12 - DNA, 59 bp, synthetic construct
  misc_feature 1..59: note = Synthetic
  source 1..59: mol_type = other DNA
ttaatacgactcactataggnnnnnnnnnnnnnnnnnnnngttttagagctagaaatag

SEQ ID NO: 13 - DNA, 22 bp, synthetic construct
  misc_feature 1..22: note = Synthetic
  source 1..22: mol_type = other DNA
aaaagcaccgactcggtgccac

SEQ ID NO: 14 - DNA, 20 bp, synthetic construct
  misc_feature 1..20: note = Synthetic
  source 1..20: mol_type = other DNA
gtgtgccatataaggtcctc

SEQ ID NO: 15 - DNA, 21 bp, synthetic construct
  misc_feature 1..21: note = Synthetic
  source 1..21: mol_type = other DNA
aatgggtttcagggggctaac

SEQ ID NO: 16 - DNA, 22 bp, synthetic construct
  misc_feature 1..22: note = Synthetic
  source 1..22: mol_type = other DNA
ggcatgatttaagctctcctcc

SEQ ID NO: 17 - DNA, 59 bp, synthetic construct; identical to SEQ ID NO: 12
  misc_feature 1..59: note = Synthetic
  source 1..59: mol_type = other DNA

SEQ ID NO: 18 - DNA, 22 bp, synthetic construct
  misc_feature 1..22: note = Synthetic
  source 1..22: mol_type = other DNA
aaaagcaccgactcggtgccac

SEQ ID NO: 19 - DNA, 20 bp, synthetic construct
  misc_feature 1..20: note = Synthetic
  source 1..20: mol_type = other DNA
ccagatatccttgtcatgtg

SEQ ID NO: 20 - DNA, 20 bp, synthetic construct
  misc_feature 1..20: note = Synthetic
  source 1..20: mol_type = other DNA
gatatccttgtcatgtgtgg

SEQ ID NO: 21 - DNA, 127 bp, synthetic construct
  misc_feature 1..127: note = Synthetic
  source 1..127: mol_type = other DNA
cgtgtgtgagtatgtacgtgcgtgtacatgtatgcatgtgcaggagagtacagagaaggaaaaatacctgtggtggaatcacgcccccgcacttgacaaggatatctggccgccccagggcggtgag

SEQ ID NO: 22 - DNA, 20 bp, synthetic construct
  misc_feature 1..20: note = Synthetic
  source 1..20: mol_type = other DNA
aaggtcgcagacctcgtctt

SEQ ID NO: 23 - DNA, 20 bp, synthetic construct
  misc_feature 1..20: note = Synthetic
  source 1..20: mol_type = other DNA
gattcccaaggaccgcactc

SEQ ID NO: 24 - DNA, 59 bp, synthetic construct; identical to SEQ ID NO: 12
  misc_feature 1..59: note = Synthetic
  source 1..59: mol_type = other DNA

SEQ ID NO: 25 - DNA, 22 bp, synthetic construct
  misc_feature 1..22: note = Synthetic
  source 1..22: mol_type = other DNA
aaaagcaccgactcggtgccac

SEQ ID NO: 26 - DNA, 20 bp, synthetic construct
  misc_feature 1..20: note = Synthetic
  source 1..20: mol_type = other DNA
ttgaggtagcttttactggt

SEQ ID NO: 27 - DNA, 20 bp, synthetic construct
  misc_feature 1..20: note = Synthetic
  source 1..20: mol_type = other DNA
gggccagtctggttctatgt

SEQ ID NO: 28 - DNA, 20 bp, synthetic construct
  misc_feature 1..20: note = Synthetic
  source 1..20: mol_type = other DNA
tcgataccatcgaccccaaa

SEQ ID NO: 29 - DNA, 59 bp, synthetic construct; identical to SEQ ID NO: 12
  misc_feature 1..59: note = Synthetic
  source 1..59: mol_type = other DNA

SEQ ID NO: 30 - DNA, 22 bp, synthetic construct
  misc_feature 1..22: note = Synthetic
  source 1..22: mol_type = other DNA
aaaagcaccgactcggtgccac

SEQ ID NO: 31 - DNA, 20 bp, synthetic construct
  misc_feature 1..20: note = Synthetic
  source 1..20: mol_type = other DNA
ttcgctcagactcacagctt

SEQ ID NO: 32 - DNA, 121 bp, synthetic construct
  misc_feature 1..121: note = Synthetic
  source 1..121: mol_type = other DNA
gtctgtgttggcccagctcccaccagtaaaagctacctcaacatggatgccatcatggaagccattaagaaaaccagggcccaaactgtgagtctgagcgaacctatctactgcagctgtt

SEQ ID NO: 33 - DNA, 20 bp, synthetic construct
  misc_feature 1..20: note = Synthetic
  source 1..20: mol_type = other DNA
gggccagtctggttctatgt

SEQ ID NO: 34 - DNA, 20 bp, synthetic construct
  misc_feature 1..20: note = Synthetic
  source 1..20: mol_type = other DNA
tcgataccatcgaccccaaa

SEQ ID NO: 35 - DNA, 20 bp, synthetic construct
  misc_feature 1..20: note = Synthetic
  source 1..20: mol_type = other DNA
atcatgtaagaacctcccca

SEQ ID NO: 36 - DNA, 20 bp, synthetic construct
  misc_feature 1..20: note = Synthetic
  source 1..20: mol_type = other DNA
tactggataaatgaagatgg